SEQUENCE LISTING (1120 sequences)

SEQ ID NO: 1 - DNA, 25 bp, Artificial Sequence
  Synthetic
gattcgaggaaccaggccttggtgt

SEQ ID NO: 2 - DNA, 29 bp, Artificial Sequence
  Synthetic
atgacgtggcagacagcggacccaggtcc

SEQ ID NO: 3 - DNA, 29 bp, Artificial Sequence
  Synthetic
atgacgtggcagaccgcggacccaggtcc

SEQ ID NO: 4 - DNA, 28 bp, Artificial Sequence
  Synthetic
cggaggaagcgttagtctgccacgtcat

SEQ ID NO: 5 - DNA, 15 bp, Artificial Sequence
  Synthetic
  misc_feature (4)..(4): The residue at this position is linked to a spacer bearing a Cy3 dye.
taacgcttcctgccg

SEQ ID NO: 6 - DNA, 21 bp, Artificial Sequence
  Synthetic
atattcataggaaacaccaag

SEQ ID NO: 7 - DNA, 30 bp, Artificial Sequence
  Synthetic
aacgaggcgcacagatgatattttctttaa

SEQ ID NO: 8 - DNA, 30 bp, Artificial Sequence
  Synthetic
atcgtccgcctctgatattttctttaatgg

SEQ ID NO: 9 - DNA, 41 bp, Artificial Sequence
  Synthetic
cttacttgaccttgggcccagttatttaaccttctagacct

SEQ ID NO: 10 - DNA, 36 bp, Artificial Sequence
  Synthetic
cgcgccgaggatcagtttcttcatctctaaaatgga

SEQ ID NO: 11 - DNA, 36 bp, Artificial Sequence
  Synthetic
cgcgccgaggctcagtttcttcatctctaaaatgga

SEQ ID NO: 12 - DNA, 30 bp, Artificial Sequence
  Synthetic
tgtatccattttagagatgaagaaactgag

SEQ ID NO: 13 - DNA, 42 bp, Artificial Sequence
  Synthetic
ggtctagaaggttaaataactgggcccaaggtcaagtaaggg

SEQ ID NO: 14 - DNA, 30 bp, Artificial Sequence
  Synthetic
tgtatccattttagagatgaagaaactgat

SEQ ID NO: 15 - DNA, 42 bp, Artificial Sequence; identical to SEQ ID NO: 13
  Synthetic

SEQ ID NO: 16 - DNA, 37 bp, Artificial Sequence
  Synthetic
  misc_feature (3)..(3): The residue at this position is linked to a Z28 quenching group.
tctagccggttttccggctgagagtctgccacgtcat

SEQ ID NO: 17 - DNA, 33 bp, Artificial Sequence
  Synthetic
  misc_feature (3)..(3): The residue at this position is linked to a Z28 quenching group.
tcttcggccttttggccgagagacctcggcgcg

SEQ ID NO: 18 - DNA, 41 bp, Artificial Sequence
  Synthetic
caagctccacactggaagaatcaggagcaatagatttcttt

SEQ ID NO: 19 - DNA, 29 bp, Artificial Sequence
  Synthetic
cgcgccgaggcttgctctcagaaggaaac

SEQ ID NO: 20 - DNA, 33 bp, Artificial Sequence
  Synthetic
atgacgtggcagacgttgctctcagaaggaaac

SEQ ID NO: 21 - DNA, 65 bp, Artificial Sequence
  Synthetic
ggtggtttccttctgagagcaagaagaaatctattgctcctgattcttccagtgtggagcttgga

SEQ ID NO: 22 - DNA, 65 bp, Artificial Sequence
  Synthetic
ggtggtttccttctgagagcaacaagaaatctattgctcctgattcttccagtgtggagcttgga

SEQ ID NO: 23 - DNA, 39 bp, Artificial Sequence
  Synthetic
gcaatgaaattgcacattttacccagccatatgccatgt

SEQ ID NO: 24 - DNA, 29 bp, Artificial Sequence
  Synthetic
atgacgtggcagacggcagccaacatcag

SEQ ID NO: 25 - DNA, 26 bp, Artificial Sequence
  Synthetic
cgcgccgaggagcagccaacatcagt

SEQ ID NO: 26 - DNA, 60 bp, Artificial Sequence
  Synthetic
aaacactgatgttggctgcccatggcatatggctgggtaaaatgtgcaatttcattgctt

SEQ ID NO: 27 - DNA, 60 bp, Artificial Sequence
  Synthetic
aaacactgatgttggctgctcatggcatatggctgggtaaaatgtgcaatttcattgctt

SEQ ID NO: 28 - DNA, 47 bp, Artificial Sequence
  Synthetic
ggagagatactaggcactcacttcatacaaaagaaaaaccaatgctt

SEQ ID NO: 29 - DNA, 32 bp, Artificial Sequence
  Synthetic
cgcgccgaggataggccttataaatgactctc

SEQ ID NO: 30 - DNA, 36 bp, Artificial Sequence
  Synthetic
atgacgtggcagacgtaggccttataaatgactctc

SEQ ID NO: 31 - DNA, 74 bp, Artificial Sequence
  Synthetic
ctttgagagtcatttataaggcctatagcattggtttttcttttgtatgaagtgagtgcctagtatctctccac

SEQ ID NO: 32 - DNA, 74 bp, Artificial Sequence
  Synthetic
ctttgagagtcatttataaggcctacagcattggtttttcttttgtatgaagtgagtgcctagtatctctccac

SEQ ID NO: 33 - DNA, 58 bp, Artificial Sequence
  Synthetic
agcactttacaatggttcagcttccaatttatatgccagatattactaaatacagagt

SEQ ID NO: 34 - DNA, 38 bp, Artificial Sequence
  Synthetic
atgacgtggcagacgtaccgtactctataaagtaaatg

SEQ ID NO: 35 - DNA, 35 bp, Artificial Sequence
  Synthetic
cgcgccgaggataccgtactctataaagtaaatgc

SEQ ID NO: 36 - DNA, 88 bp, Artificial Sequence
  Synthetic
agttgcatttactttatagagtacggtacctctgtatttagtaatatctggcatataaattggaagctgaaccattgtaaagtgctaa

SEQ ID NO: 37 - DNA, 88 bp, Artificial Sequence
  Synthetic
agttgcatttactttatagagtacggtatctctgtatttagtaatatctggcatataaattggaagctgaaccattgtaaagtgctaa

SEQ ID NO: 38 - DNA, 32 bp, Artificial Sequence
  Synthetic
ccctcatcctggttcagaaataaccgcgtggt

SEQ ID NO: 39 - DNA, 26 bp, Artificial Sequence
  Synthetic
cgcgccgaggcattgctgttgttgcc

SEQ ID NO: 40 - DNA, 30 bp, Artificial Sequence
  Synthetic
atgacgtggcagacgattgctgttgttgcc

SEQ ID NO: 41 - DNA, 53 bp, Artificial Sequence
  Synthetic
caacggcaacaacagcaatgccacgcggttatttctgaaccaggatgagggtg

SEQ ID NO: 42 - DNA, 53 bp, Artificial Sequence
  Synthetic
caacggcaacaacagcaatcccacgcggttatttctgaaccaggatgagggtg

SEQ ID NO: 43 - DNA, 34 bp, Artificial Sequence
  Synthetic
gtcctcaatgatgggagggcattcacctctacat

SEQ ID NO: 44 - DNA, 30 bp, Artificial Sequence
  Synthetic
cgcgccgaggagaaagaagacagatggtca

SEQ ID NO: 45 - DNA, 33 bp, Artificial Sequence
  Synthetic
atgacgtggcagacggaaagaagacagatggtc

SEQ ID NO: 46 - DNA, 59 bp, Artificial Sequence
  Synthetic
aggttgaccatctgtcttctttcttgtagaggtgaatgccctcccatcattgaggacag

SEQ ID NO: 47 - DNA, 59 bp, Artificial Sequence
  Synthetic
aggttgaccatctgtcttctttcctgtagaggtgaatgccctcccatcattgaggacag

SEQ ID NO: 48 - DNA, 33 bp, Artificial Sequence
  Synthetic
gccaggcttgtaaattacatgagcagccctctt

SEQ ID NO: 49 - DNA, 32 bp, Artificial Sequence
  Synthetic
atgacgtggcagacgcaaagggagtctaaacc

SEQ ID NO: 50 - DNA, 28 bp, Artificial Sequence
  Synthetic
cgcgccgaggccaaagggagtctaaacc

SEQ ID NO: 51 - DNA, 56 bp, Artificial Sequence
  Synthetic
ttcaggtttagactccctttgcagagggctgctcatgtaatttacaagcctggcag

SEQ ID NO: 52 - DNA, 56 bp, Artificial Sequence
  Synthetic
ttcaggtttagactccctttggagagggctgctcatgtaatttacaagcctggcag

SEQ ID NO: 53 - DNA, 49 bp, Artificial Sequence
  Synthetic
gtgacacaggccagtaagttactcaaattttaagtttgagctttttcaa

SEQ ID NO: 54 - DNA, 31 bp, Artificial Sequence
  Synthetic
cgcgccgaggtttgtaagatggaacaatcgt

SEQ ID NO: 55 - DNA, 35 bp, Artificial Sequence
  Synthetic
atgacgtggcagaccttgtaagatggaacaatcgt

SEQ ID NO: 56 - DNA, 75 bp, Artificial Sequence
  Synthetic
tgtcacgattgttccatcttacaaatgaaaaagctcaaacttaaaatttgagtaacttactggcctgtgtcacat

SEQ ID NO: 57 - DNA, 75 bp, Artificial Sequence
  Synthetic
tgtcacgattgttccatcttacaagtgaaaaagctcaaacttaaaatttgagtaacttactggcctgtgtcacat

SEQ ID NO: 58 - DNA, 42 bp, Artificial Sequence
  Synthetic
gtgttacaagctgcctctccaaaatcaatgccttcactatat

SEQ ID NO: 59 - DNA, 31 bp, Artificial Sequence
  Synthetic
atgacgtggcagacgaacttgcctgaagcaa

SEQ ID NO: 60 - DNA, 26 bp, Artificial Sequence
  Synthetic
cgcgccgaggcaacttgcctgaagca

SEQ ID NO: 61 - DNA, 64 bp, Artificial Sequence
  Synthetic
gtcattgcttcaggcaagttctatagtgaaggcattgattttggagaggcagcttgtaacacgt

SEQ ID NO: 62 - DNA, 64 bp, Artificial Sequence
  Synthetic
gtcattgcttcaggcaagttgtatagtgaaggcattgattttggagaggcagcttgtaacacgt

SEQ ID NO: 63 - DNA, 44 bp, Artificial Sequence
  Synthetic
gccttttcaaatcgactttctcaaatgttttgcctgttctctct

SEQ ID NO: 64 - DNA, 27 bp, Artificial Sequence
  Synthetic
cgcgccgaggatttccattcccagtgc

SEQ ID NO: 65 - DNA, 31 bp, Artificial Sequence
  Synthetic
atgacgtggcagacgtttccattcccagtgc

SEQ ID NO: 66 - DNA, 66 bp, Artificial Sequence
  Synthetic
taatgcactgggaatggaaatgagagaacaggcaaaacatttgagaaagtcgatttgaaaaggcag

SEQ ID NO: 67 - DNA, 66 bp, Artificial Sequence
  Synthetic
taatgcactgggaatggaaacgagagaacaggcaaaacatttgagaaagtcgatttgaaaaggcag

SEQ ID NO: 68 - DNA, 50 bp, Artificial Sequence
  Synthetic
ggtattgtgtgtccagttttgtttgtaaaatgtaaccttcgtgtgaatgt

SEQ ID NO: 69 - DNA, 32 bp, Artificial Sequence
  Synthetic
cgcgccgaggaccatctattcctttctttttg

SEQ ID NO: 70 - DNA, 36 bp, Artificial Sequence
  Synthetic
atgacgtggcagacgccatctattcctttctttttg

SEQ ID NO: 71 - DNA, 77 bp, Artificial Sequence
  Synthetic
cttccaaaaagaaaggaatagatggtcattcacacgaaggttacattttacaaacaaaactggacacacaataccat

SEQ ID NO: 72 - DNA, 77 bp, Artificial Sequence
  Synthetic
cttccaaaaagaaaggaatagatggccattcacacgaaggttacattttacaaacaaaactggacacacaataccat

SEQ ID NO: 73 - DNA, 28 bp, Artificial Sequence
  Synthetic
accacctgcctctcatccatgcagcaac

SEQ ID NO: 74 - DNA, 32 bp, Artificial Sequence
  Synthetic
cgcgccgaggttcatcaggcctgtatataaaa

SEQ ID NO: 75 - DNA, 36 bp, Artificial Sequence
  Synthetic
atgacgtggcagacatcatcaggcctgtatataaaa

SEQ ID NO: 76 - DNA, 55 bp, Artificial Sequence
  Synthetic
cttgttttatatacaggcctgatgaattgctgcatggatgagaggcaggtggtgg

SEQ ID NO: 77 - DNA, 55 bp, Artificial Sequence
  Synthetic
cttgttttatatacaggcctgatgatttgctgcatggatgagaggcaggtggtgg

SEQ ID NO: 78 - DNA, 29 bp, Artificial Sequence
  Synthetic
gccaagcagtggtcatgaaagtccagcct

SEQ ID NO: 79 - DNA, 31 bp, Artificial Sequence
  Synthetic
atgacgtggcagacgctgtcacatccttgag

SEQ ID NO: 80 - DNA, 27 bp, Artificial Sequence
  Synthetic
cgcgccgaggcctgtcacatccttgag

SEQ ID NO: 81 - DNA, 51 bp, Artificial Sequence
  Synthetic
gttcctcaaggatgtgacagcggctggactttcatgaccactgcttggcca

SEQ ID NO: 82 - DNA, 51 bp, Artificial Sequence
  Synthetic
gttcctcaaggatgtgacaggggctggactttcatgaccactgcttggcca

SEQ ID NO: 83 - DNA, 45 bp, Artificial Sequence
  Synthetic
ctctgtgccatcttttactccatgaactgcatttaatgtgtagct

SEQ ID NO: 84 - DNA, 30 bp, Artificial Sequence
  Synthetic
cgcgccgaggatctgcatttttcaactggt

SEQ ID NO: 85 - DNA, 33 bp, Artificial Sequence
  Synthetic
atgacgtggcagacgtctgcatttttcaactgg

SEQ ID NO: 86 - DNA, 70 bp, Artificial Sequence
  Synthetic
agacaccagttgaaaaatgcagatgctacacattaaatgcagttcatggagtaaaagatggcacagagct

SEQ ID NO: 87 - DNA, 70 bp, Artificial Sequence
  Synthetic
agacaccagttgaaaaatgcagacgctacacattaaatgcagttcatggagtaaaagatggcacagagct

SEQ ID NO: 88 - DNA, 43 bp, Artificial Sequence
  Synthetic
tgcgcaaactggtttaatatcattagtgtaacagccaaggtgt

SEQ ID NO: 89 - DNA, 35 bp, Artificial Sequence
  Synthetic
cgcgccgaggatcaaaggcagtaaattataaactt

SEQ ID NO: 90 - DNA, 38 bp, Artificial Sequence
  Synthetic
atgacgtggcagacctcaaaggcagtaaattataaact

SEQ ID NO: 91 - DNA, 73 bp, Artificial Sequence
  Synthetic
ctacaagtttataatttactgcctttgatcaccttggctgttacactaatgatattaaaccagtttgcgcaat

SEQ ID NO: 92 - DNA, 73 bp, Artificial Sequence
  Synthetic
ctacaagtttataatttactgcctttgagcaccttggctgttacactaatgatattaaaccagtttgcgcaat

SEQ ID NO: 93 - DNA, 44 bp, Artificial Sequence
  Synthetic
agcatcaagcctgctactaaaaatattttttcctgctgctctgt

SEQ ID NO: 94 - DNA, 30 bp, Artificial Sequence
  Synthetic
cgcgccgaggagaatgtgtgttcttccatc

SEQ ID NO: 95 - DNA, 33 bp, Artificial Sequence
  Synthetic
atgacgtggcagacggaatgtgtgttcttccat

SEQ ID NO: 96 - DNA, 69 bp, Artificial Sequence
  Synthetic
tttagatggaagaacacacattctcagagcagcaggaaaaaatatttttagtagcaggcttgatgctta

SEQ ID NO: 97 - DNA, 69 bp, Artificial Sequence
  Synthetic
tttagatggaagaacacacattcccagagcagcaggaaaaaatatttttagtagcaggcttgatgctta

SEQ ID NO: 98 - DNA, 44 bp, Artificial Sequence; identical to SEQ ID NO: 63
  Synthetic

SEQ ID NO: 99 - DNA, 27 bp, Artificial Sequence
  Synthetic
cgcgccgaggatttccattcccagtgc

SEQ ID NO: 100 - DNA, 31 bp, Artificial Sequence; identical to SEQ ID NO: 65
  Synthetic

SEQ ID NO: 101 - DNA, 66 bp, Artificial Sequence; identical to SEQ ID NO: 66
  Synthetic

SEQ ID NO: 102 - DNA, 66 bp, Artificial Sequence; identical to SEQ ID NO: 67
  Synthetic

SEQ ID NO: 103 - DNA, 35 bp, Artificial Sequence
  Synthetic
ttgagtaacaaagattgggcccttatacctgtgaa

SEQ ID NO: 104 - DNA, 30 bp, Artificial Sequence
  Synthetic
atgacgtggcagaccgtgtctggcaatgac

SEQ ID NO: 105 - DNA, 26 bp, Artificial Sequence
  Synthetic
cgcgccgaggtgtgtctggcaatgac

SEQ ID NO: 106 - DNA, 56 bp, Artificial Sequence
  Synthetic
ctttgtcattgccagacacgtcacaggtataagggcccaatctttgttactcaaat

SEQ ID NO: 107 - DNA, 56 bp, Artificial Sequence
  Synthetic
ctttgtcattgccagacacatcacaggtataagggcccaatctttgttactcaaat

SEQ ID NO: 108 - DNA, 55 bp, Artificial Sequence
  Synthetic
caaatggcatttcaaatgcataaaaataacttattcgtaaattttctttctctca

SEQ ID NO: 109 - DNA, 33 bp, Artificial Sequence
  Synthetic
cgcgccgaggtttcttgttttagtatagcacct

SEQ ID NO: 110 - DNA, 37 bp, Artificial Sequence
  Synthetic
atgacgtggcagaccttcttgttttagtatagcacct

SEQ ID NO: 111 - DNA, 83 bp, Artificial Sequence
  Synthetic
ttcaaggtgctatactaaaacaagaaagagagaaagaaaatttacgaataagttatttttatgcatttgaaatgccatttgga

SEQ ID NO: 112 - DNA, 83 bp, Artificial Sequence
  Synthetic
ttcaaggtgctatactaaaacaagaaggagagaaagaaaatttacgaataagttatttttatgcatttgaaatgccatttgga

SEQ ID NO: 113 - DNA, 537 bp, Artificial Sequence
  Synthetic
  misc_feature (275)..(275): n is c or t.
aagttagaagaaccaagactatcttgtcaggggtgtattttgagagtggcagacttttcagtgcctttccattcatgacacttcttgaatctctggcagaaccagccagccgtgttcacagtgtcaaatgaagggatgtctttgattgcttccaggtgttcctcagcaccaccggagggggatgggtgatcagccgaatctttgactcgggctacccatgggacatggtgttcatgacacgctttcagaacatgttgagaaattccctcccaacnccaattgtgacttggttgatggagcgaaagataaacaactggctcaatcatgcaaattacggcttaataccagaagacaggtaaatataatgtgactgccaagggcttttaggaagaaggagcctctgcctgtccagcagcctatacaagccaggcagtaccacagcaacatggctgaatgtgtgggaacacttgatacaaatttgcttgataataacagctaactgttcttaagtactcagaaagtgaaattatgtatttc

SEQ ID NO: 114 - DNA, 18 bp, Artificial Sequence
  Synthetic
cgggctacccatgggaca

SEQ ID NO: 115 - DNA, 31 bp, Artificial Sequence
  Synthetic
tctggtattaagccgtaatttgcatgattga

SEQ ID NO: 116 - DNA, 19 bp, Artificial Sequence
  Synthetic
ctgttcttcctgaagcctc

SEQ ID NO: 117 - DNA, 17 bp, Artificial Sequence
  Synthetic
ttgaggttggtgccttc

SEQ ID NO: 118 - DNA, 19 bp, Artificial Sequence
  Synthetic
aagagtgtattgagagcct

SEQ ID NO: 119 - DNA, 20 bp, Artificial Sequence
  Synthetic
tcagccttaaaaagacctcc

SEQ ID NO: 120 - DNA, 19 bp, Artificial Sequence
  Synthetic
ctcgtcacttcctctgtcc

SEQ ID NO: 121 - DNA, 16 bp, Artificial Sequence
  Synthetic
gggagaagtgcggcac

SEQ ID NO: 122 - DNA, 18 bp, Artificial Sequence
  Synthetic
gaccttatgtgtttttcc

SEQ ID NO: 123 - DNA, 22 bp, Artificial Sequence
  Synthetic
caatttcctcaaaagactttcc

SEQ ID NO: 124 - DNA, 20 bp, Artificial Sequence
  Synthetic
aaggacttaagaattgtcac

SEQ ID NO: 125 - DNA, 17 bp, Artificial Sequence
  Synthetic
cctcaatccttcaccgc

SEQ ID NO: 126 - DNA, 19 bp, Artificial Sequence
  Synthetic
gaggtagtgtttacagccc

SEQ ID NO: 127 - DNA, 22 bp, Artificial Sequence
  Synthetic
tcacatctcgagtgataatctc

SEQ ID NO: 128 - DNA, 17 bp, Artificial Sequence
  Synthetic
tgatgggagacgagttc

SEQ ID NO: 129 - DNA, 18 bp, Artificial Sequence
  Synthetic
tgcacacacacacatacc

SEQ ID NO: 130 - DNA, 15 bp, Artificial Sequence
  Synthetic
aggtcccctccgctc

SEQ ID NO: 131 - DNA, 16 bp, Artificial Sequence
  Synthetic
cacagtggtgttggac

SEQ ID NO: 132 - DNA, 18 bp, Artificial Sequence
  Synthetic
atcctgaagagcaagtcc

SEQ ID NO: 133 - DNA, 18 bp, Artificial Sequence
  Synthetic
attccggtttggttctcc

SEQ ID NO: 134 - DNA, 17 bp, Artificial Sequence
  Synthetic
ctgaaacccaggactcc

SEQ ID NO: 135 - DNA, 19 bp, Artificial Sequence
  Synthetic
ggaacaatcaccttttctc

SEQ ID NO: 136 - DNA, 122 bp, Artificial Sequence
  Synthetic
aggctcccttagatgcctgacattctgttcttcctgaagcctcactcccttctctcctggctgcagacacgtccccatcagaaggcaccaacctcaacgcgcccaacagcctgggtgtcagc

SEQ ID NO: 137 - DNA, 122 bp, Artificial Sequence
  Synthetic
taaaatcatttatttatttatccatccatcaagagtgtattgagagcctgacaacataccaggcatcaagccctggaggtctttttaaggctgagccaatatagctatggataacattctaa

SEQ ID NO: 138 - DNA, 91 bp, Artificial Sequence
  Synthetic
ccctcgtcacttcctctgtcctgtggggtgggggtgcaggcgctctctcctttagctgtgccgcacttctccctacaggccaggagaaaca

SEQ ID NO: 139 - DNA, 122 bp, Artificial Sequence
  Synthetic
cttctgtggaccttatgtgtttttcctctttgctggagtgctcctggcctttaccctgttctacattttttaaagttccagaaaccaaaggaaagtcttttgaggaaattgctgcagaattc

SEQ ID NO: 140 - DNA, 122 bp, Artificial Sequence
  Synthetic
tggttaaggacttaagaattgtcacttgtgtgtgtatattgttgttgttgttgcaacggtgtctgtgtacgcacggttacagtggatcaaatttggggagttaggaagtggcgttggtttgt

SEQ ID NO: 141 - DNA, 91 bp, Artificial Sequence
  Synthetic
cgaggtagtgtttacagccctcatgaacagcaaaggcgtgagcctcttcgagcatcatcaaccctgagattatcactcgagatgtgagtac

SEQ ID NO: 142 - DNA, 91 bp, Artificial Sequence
  Synthetic
ggagacgagttcaaggtgagtgggtggggctgggctgctaggggaatccagatggcatgtggtatgtgtgtgtgtgcacacgcatggggag

SEQ ID NO: 143 - DNA, 122 bp, Artificial Sequence
  Synthetic
cagccacaggtcccctccgctcaggtgatggacttcctgtttgagaagtggaagctctacaggtgaccagtgtcaccacaacctgagcctgctgccccctcccacgggtgagccccccaccc

SEQ ID NO: 144 - DNA, 122 bp, Artificial Sequence
  Synthetic
agagcaagtcccccaaggaggagctgctgaagatgtggggggaggagctgaccagtgaagacaagtgtctttgaagtctttgttctttacctctcgggagaaccaaaccggaatggtcacaa

SEQ ID NO: 145 - DNA, 122 bp, Artificial Sequence
  Synthetic
ggaactgaaacccaggactccgtctcttgccagtgaaagttatgttaggaagcagtgaggtggtctaaagcagtatgaaaggcaaagagaaaaggtgattgttccctcttgaatggcccttg

SEQ ID NO: 146 - DNA, 1021 bp, Homo sapiens
  misc_feature (561)..(561): n can be g or t.
cactagaccgcctgtccccaagggagcctcagtggggcgacagggtgctcggcggactccacctcaggccctccccactgttgctgtgcattcctgtgcaggtgcatctctttcttactaactggtatttattaagggaggtgctctgtaggtctggagcctttccctcatcctttttgcgagtccccacctttttgtttttttttttttctttgaggctcactagaggacgcagaaccttgggagattgatttgcacagaactccccacctcccacttttacaatttccagtttctgattgaaaattttagggtttctccccactgcccttccctatctttccttcccctcaacaccatgaaggaaaaacacacacggcagggctttttgtagccctgaaggcaactttagacatttaaaatccagcactttaatctcttgttctctgtgaatcactatgagaagtgaatggttttaaaggctgtaatgctatgttggaaattggtttgttttgccttttattgaaaaggtaagatcatgtgattggaagaacacaactnttggcttgggaagaggactttgctgctgaagtgttttctaccttctgagtgtgtttaaggcaggatttggagggaaggaccagcttagggagagtgtctgagccacagcgtcaggatgggggaaaccacatgggatccatcaagttccagttgaacaggagcaagatcagaacttaggagggcagtgtcagctcccttgttggctgtcaaggaacaccgatctagtagaaacccacttggttgtgacccaggtagaggtagatgccatacatttgagatatgcgtccttaaggaacctgacaagcagactgaagggatggtaagtgtgacagcctgataagttttctcaaagcccaggatacagagccagtgttttctgtaactggagacctcagttaggccaacttcgaattccagagcaacgtaggaagtctattcagcagaaactcgacattgttca

SEQ ID NO: 147 - DNA, 1021 bp, Homo sapiens
  misc_feature (561)..(561): n can be c or t.
ataccaaaagtaattgtagtactgaattttgctgtcatttaagccaatggtttgcactgaaactctgtagacaactctgatactgccattccctgttcttactgcctacaatgatagtgagcacaccaagtagcaatcacctgttcattgttttcttacatagactttaggtccctatggtttactaaaggctggcagataataagtattcaataatatgtcttaaggcattttaatactctagatgctctgaatcctaatctcaaaaggattaactttaaaatagaagttagaagaaccaagactatcttgtcaggggtgtattttgagagtggcagacttttcagtgcctttccattcatgacacttcttgaatctctggcagaaccagccagccgtgttcacagtgtcaaatgaagggatgtctttgattgcttccaggtgttcctcagcaccaccggagggggatgggtgatcagccgaatctttgactcgggctacccatgggacatggtgttcatgacacgctttcagaacatgttgagaaattccctcccaacnccaattgtgacttggttgatggagcgaaagataaacaactggctcaatcatgcaaattacggcttaataccagaagacaggtaaatataatgtgactgccaagggcttttaggaagaaggagcctctgcctgtccagcagcctatacaagccaggcagtaccacagcaacatggctgaatgtgtgggaacacttgatacaaatttgcttgataataacagctaactgttcttaagtactcagaaagtgaaattatgtatttcaccttgtcagcaacactttacgtattattataataatccttttattatggagaaactgaaacagcaaaattcagccatttacccaagctcactgagtagtaagtgaactctgtgaccttggcaagttacttgatcctcagctgtagcaaccaaaagagaatgatttgtctatgactttgttgataaaagaaacacact

SEQ ID NO: 148 - DNA, 1021 bp, Homo sapiens
  misc_feature (438)..(438): n is a, c, g, or t
  misc_feature (504)..(504): n is a, c, g, or t
  misc_feature (561)..(561): n can be c or t.
cagctgtggggtcaggaagggcttgaagtatgggacactagcctgccccacctccactctgcagcacccacaggaccaccctcatgcccctggcaacagcatgcagggcagctgcaggatccaggtgggacccagatactatatgaaggagccaccttacctgctttttgcaaagctactgggatggcataggcaggtccaatgcccatgatgtcaggtgggaccccaaccactgcataagacctcaggaccccaaggatgggaaggcccaactcttctgccttggacctccgggccagcaggatggcagctgccccatcactcacctggctagagtttcctaggggcaaactgttggggtaagaaggcatcggggtggggatgaggagatcccagccctcccacttctactttgcagaggggcctggtctattccangttcccagagtacagcacccagcatggccatggcctgctttctcatacccctaccccggaccagtntcaccagctgtggtagaaccatctttcttgaaggcaggcttcagtttggccaggccntccatggtggtgctggggcggataccctcatcctgggtcacagtgatgctcctcttggtgcccttgtcatcatggaccgtggtggtcacaggcacaatctcagcttggaaacagcccttgctctgggctcttgctgccctgccagcaccatggacagccagcttcagactcccttggggttcccttccttccctgcccccaacccctatccatttgggtagacacaagctcaggctgctaaattcagggacatgctcgactttgggggagctctgagggcatggctaaggccttacagggccttcttcaccatcagccccagacctccagatcgtggccaatcccaacctcaaaggggggaaagggtgtttggaagtggtgcctccacttagagccctttgtccaagagggattaagcctgcttgattctctctgctaaactgaggatggaaccccagaa

SEQ ID NO: 149 - DNA, 1021 bp, Homo sapiens
  misc_feature (561)..(561): n can be g or a.
gaggatcaaagcacctggtacaatgcctggccagaaagttgaataatcgaatatagctaacgtcactattgcaggctggctatgtgcctggcggtgttcttagccatttacaagtatgaactcatttaatcctcataagatcctgtatgaggtgagtaagctgttaattcccttccttgcccatactctgtgactccaacccaccacagttgaatttctccttatgaattataaatcagaaaacggccccaaattctgtcatgtctaagtgggaaaatggaagaaggcattgatttctcccctactcaagcagaagagaattaacctcagtccctgctttgcccatattccttccccagggccccaggaagaagacatggaaaaacaatatttccaccaaagtttatttctctgaaacaatcaccagttgctgtcctctatggcacactgagagccccaggagggtctttaactcccttcctcagattatattcatcccagaaatatagccttggacaataatttggttacagcatagtcccaggaatgaggtcccccaanttgctaagttttacataggggagactgggaaattcaaagaattggatggagaaaccataggatccaagataatgtcagggggttgaagatgttggagaggcatggtagcatcattgagtttgaatctccttctcacttggagtggaagttgtaggattctgcctctaggaaatgtgccatcctacagaataaataaaagggagataatgaggcttcaacccaacttgcccccatcgtttgtcactgtaaccatcccatgccttaatacagtgatactgaaaactccagggcaccaacaactaatacaaaggaagcaccttcagcctcctctccacagacatcccacttggtagaagaggaggatgctccttcctgctcttaatcctagcaatggcagcttaaatcatgcccttgcctagatcctcatggaagctcacccatataataatcaagattagtt

SEQ ID NO: 150 - DNA, 1021 bp, Homo sapiens
  misc_feature (561)..(561): n can be c or a.
  misc_feature (933)..(933): n is a, c, g, or t
aggtgcactttttccaggacctcctgcacaggtgtgatatttagcctggaagcaatgtgtacatggaatgccctacaggcacaggaggcatccctggagactgaatggtgtctgggaagagtagggccacagagctgagcccctatggactgcagcagagggcctggctccaatcctagcctaccatatcccagtcccatgatcgtgagtagtcccatgggatcaagtgctctcattcataaaagaagggaggtaacagctgccccactcacgccccaggatcatccggcagtcaaaggggattcaggtgcttcctggaagacagagtcacaggggaccctccttttcccagccacccatatcagtccaccttttgggttttgacctttactatgtggttttctagacttctattgacaaatcctgctttatggacagggatgcttttcatttagattgggggccactccccaacatctcatttatttttcacagctctggtcccatggagtcttgtttgagtgcaagtgaactgaatttcccaattcctcaaaaagagcnatagtaataaaaaccataatagtgacacttacatatggatagtgctttgtagtttagaaaatgctttcaccaactgattgccatgacagccctgagaagtaacctactctacagatgaggagcctagagagagaaagtgactttcctgggcacataggcccatgaggttctggtgccagcataatagactagtcaaatttccagactctggagtcagactgcctgagttcaaaccatgggtcctcttggtcaggttttataaccactctaaaactctgtttgcccatctgtaaagtgagcacaattacagaatctacctaatagggctgtctgtatgtcaatgggcttggcctgtgcctgaggaaatgctanccccatgatcctgcagccatggttaggaaggacatggcagggaatgggacctttcacagaccgggctgtggccagcagccagggccga

SEQ ID NO: 151 - DNA, 1021 bp, Homo sapiens
  misc_feature (561)..(561): n can be c or g.
tcatacaactccttgcagttcatgtaaggactcggattttacctggagtggaaaaagaagcactgaaagatttgagcaggggagtaacctgatagcgtttatgtttagtcctgccacttcgacagataaacgcaccaatgggcttgatgagatttaggccaacccataaccgcccctcaacttctttcctttcaatttcaaaactcctctatggcttcctccatctgttcttccttctgagaagtgctctctctgcccctttacagaactaaccacttcggcaactccttggacactttccttcttgttaataatttgctttctccgcccctcaaaagcttgctgtttctgtaaatcattacctgtaagaggaaccgctgggagtcctgtaaactttagcccagagcttggctcctcctccagaatgtctccaccaatcaaggaaagtgttttgggccagtcttgctcctccggattgtcagactgctcctccctcttctttagactgccacgaggaaaaagcagatgtgagaactcaaggttcagggctgctcttctaanaaacaagtctgccataatctccatctgtgttggaatctgttaactagtgagtacctcatctcccctcctgtgtaagatttcctgaactggcacatctgttttttgagcaaagataacaaacagatgaacaaaaccaacaatcaaaaatgctgtcattaaagtcttgggcagccaaagtttctctcagaatttctcagttgtgtgatactatctattaagtgatgaggagtatgcacacacaaaaggctataaatgtagcagctgagttttcatgttgagccttttggtgctatttgattttttgaaaaactatgtacatgtattaagttgataaattttttttttaattttaattgaaccagatgcggtggctcaagcctgtaatcccaccactttaggaggctatggtgggcagatgcagatcacttgaggccaggagttcgagaccagcttggccaac

SEQ ID NO: 152 - DNA, 1021 bp, Homo sapiens
  misc_feature (561)..(561): n can be a or t.
tatgtgttgaatgaaaggctgggtcatatgtgacccttgtgagcagctgtttccgtggactgctcctgggtcccctcctccacccgccctgcctctcccatttcatcctaggaggtgcctgtggccgggcgcagtagctcatgcctgtaatcccagcactttgggaggccgaggcgggcggaccacctgaggtcaggaatttgagactagccggcccaacatggcgaaaccccatctctactaaacatacaaaaaattagccaggcgtcgtggcgggcgcctgtaatcccagctactcaggaggctgaggcaggagaatcgcttgaacccaggaggcggagcttgcagtgggccgagattgcgccactgcactctagcctgggggacaacagcgaaactccgtctcaaaaatatatatatatattaattaaataaaaaaacgaggtgccttctcctgactccctgatccccgcgctctccagctctgccctcgcgatcgctggagccccctgaggaactcacgcagacgcggctgcaccgcctcatcaatcccaacttctncggctatcaggacgccccctggaagatcttcctgcgcaaagaggtgccgagcacagccgtagccaggggaggggctgaagcggggcaggggaggggctgaagcgagcagaggagggtctaggacttggggagggagcccaggaggacagaaaaaggccgggctgaaaccaggggtggggttacagccggggcggaactgcatttagggggcggggccgggtgtgaagcaaggccagggggcagtcggacagtacccactgaagccccgcccctgcaggtgttttaccccaaggacagctacagccatcctgtgcagcttgacctcctgttccggcaggtgaggtcctgtctcccctttctgcctcagtgaactcagcagggctgtgtggacgcaaagatgagctagctgcaaagcctgcctctgcatgttgggatttggggtccttgacaggggtgagga

SEQ ID NO: 153 - DNA, 1021 bp, Homo sapiens
  misc_feature (561)..(561): n can be t or c.
ctatatgcttgaaagaatttataattaaaattttttttaaaaaaagagcatgaagacttgcacagcaagatatcagaaagctaaatggaaattttcttcttagctatgtgaaagacacaggcagagcaccagatggttcagtagcctgagttctagaaataatctcaacatggtaagagggtctgtaagctagcctacacctatgcgaaacagggttttatgcatgggacactattccagtagaaaatgcaggatttgagtagacttctagagttggttttaaaatgatttaatgtaaggcatcaaatctagacaatcagtaagagagtaacccatacaggctatattttcacatgttctataaagtatagtttggtgtctacagcctgcaaaccacagccaggccccaaatctttcaagttggcccctgactctttcctgctgtctccatatgaccgagtatgcactgaactatcagcgtttccaggttcctctccaggcaccgcagagtggtggcgctctcacaaaggcatgacaggaagacagggtgtgaggttgganggagagaggctgtagctgaggaaaagcacagcccatggcattttactgtaatgcctgaacaaatgcacttaatgaatatgtggcaaatgtaggctcagaagtatcatttctttcctgtaaatgtaaatgctctccctctgaagttcctgtgggaatggcttctggattctgggggtgagtgtggggccaccctccacgaggcctctgcctacctgaaagcatcattccatagaccctcccattgttcacacacagtggacctaactctccactttcactttttcttctgtaatagtttataacagtcaatagaactcccacattagcttttagggtcatcacagaatacaaaatgttgaagatacatattttatcttttctatctttctccttagtatccaggtacactaactctgatattctaacagaaattatacagacaccatgatcaccatcttga

SEQ ID NO: 154 - DNA, 1021 bp, Homo sapiens
  misc_feature (561)..(561): n can be g or a.
ggttcactcacccctcctcccacctcggcagccctgggatgtcgctgctgactcaggaggaacccgaggtgccgtagcggctgctccaatattgcagaagaggttcctcaggcagctctgcccacagccccaagtcacgaattccgtgactccagctccatcccaggccccagggtacctggcccagggttgtgctgccgcagacttggcctgtaccatccaggcggcggtggggagctggggttggaaaggcttcttggagtggactcctgggtctgtctgggagacggggaggaagggacactctgaacatcaccaggggctgctggggggccctggccacccccagagtcagaacaggcaggtggggcaggatctcaggtcatcctatgctacactcagccattgcgtggcccctctcctccctgtgcctggccttttggccagccctggggccaccgagaggatgcagcaccgaaccctccaggagcccccagtgctgccgtctgtgggacagggacaatcccatccccactgctactgtctgtgctgtgctgggcncagagctggacacctccaaggcccagcgcccgtagtggctctcatcatggacaattcacaggcagatggtggccagctctgtggcctgcagggactgggagcggcgccagaccatctaggccccaacctatctgcattatcctggaagacttcctggaggaggcttctaagctgaggcccaaggaccatgtcaggtctaggactaggaccagtgcaggccgaggccagagagacagctgggcttccaggtagggtcaaagtgaggtgggcagcaggtgtgggggccaggggactcggggacttcctctccggctgggcccgcctgacgtgggaggcagccagggttaatcatttccacgaagccttgaccccacctgccttggcgctctgctcccgcctcccactgcccctcaggccagctcaggagccatggggcgctgggcctgggtccccagcccct

SEQ ID NO: 155 - DNA, 1021 bp, Homo sapiens
  misc_feature (561)..(561): n can be t or c.
tttatggcacaaatggggccgggggcaggcccaggggcaattcaacaggaggcaagagcccagggctccagagtggagagacaggaggcagctcagtccccagaccccagcagagcatctggggcctcggccccactccagagcttcttcctgagggagccatgcacagcaatgctgggagagggactgatggggtggggtcaggcctcctgccacagagctgggctgcagagcccagatggaaagacacagtgaagagctcaacctccttccaagctctccttctcagggcttcaggttccagagccccaggggagctcccagccaggggcagggtcaccttgatattcacaactgggcttgtgggggccatcttcagtgcaaccgttgtgacaaagtcaagaggctgcctccctgaagcagacccactgcctacgccacactgacggtccagaggccccctcctgagggcggccagcaaggggcactgtggcagctcccactgtgcctgtcccagactgggtcagcaggtctctctggacagcacactgcaccaagtangcccaccaaaaacgcatcaggtgtggccatggcccacagtaccttcttcattccctgcctctaacatgtgcggtctgaatgaattttgtcactcttctgccatttataaaggagaagacagtgatccaaagctatgcatgtttctgaagccctcaaggaagctcggtgcaggccatcacttcttttggcagaaggcgggctgtggtctctatgtacacacgcgagcccgccagtgacgtgcggcagtgcgtggcgtccaggctgggacaggggcctttcaagtctccccagggaccggtgttttctacaacagacaggtgctcccagaccgttggggtacaggccaggccgtctacaccacagtattgagggagctgcggctgtggcggccaccccctggcagtgcctctgcagctggggtgctcccgctctgggcagggtcagggggcacgagcagggc

SEQ ID NO: 156 - DNA, 1021 bp, Homo sapiens
  misc_feature (561)..(561): n can be a or c.
ttaatataaataggatatcataataaatagaaatcatgccaggtcagacgcacagcacgcttggagctcagggttccctgagaccctgaccctaagttctgctgttcccttgccctggggaccagagacggcctccagtccccctcaagtacctctgtgtgacctcacaaggcctcccagggcctcagatgtgagctgctactctgagctaccccagccccttcttacagacctttacccagaggaagagcctgggtccctcagaacctctgcacctgacttagcaacctgcccctgccctacccacctccacaaacccctgctgcaggtccagccatcagaccctggccatcccaggctgcagggaagatcacggggaagagaacgaagaacctaccaaagctttccaggcctctcctcctcccagtgtcttccttcccaggcctgaaggtggcttctctgcctccccaagagcctgaatgccaagtgacctccttctggaaacttctgccagattgttcctatgcccaagttctctgatcatcctcaaaagaagacagncttccatcccagaggcccctctctatcttccactcatcaaacttctaggggacaaggagtcctttgggatcctagcccctctggcccacctaagtcccaacctaaggggcagcaaaggcacagatggtgataatttgctgggggctggtccactcccctgggccctgctgtctcaccctgtggtcagggctcttgtagatgacttgtgtagtttgttcactgcacaaagtgagcaaggggccaaagggacaagtagaggcagaagtccagcccacgctccccagtccacaatctcccagaggaaggggcaccttcttctagctccctccctatggaagtttccactctgctcagcttcatcacagcccagcccagagtggagtggactggccaggcaccctcggggtctgccagcagcccccatttgggtttagcgatgccctgggccccagccaccctt

SEQ ID NO: 157 - DNA, 1021 bp, Homo sapiens
  misc_feature (561)..(561): n can be a or c.
tgtgacaatcagcaaagccccacccaggcccccatctgggatgatgggagagctctggcagatgtcccaatcctggaggtcatccattaggaattaaattctccagcctcactctcggctctttcctacttgttagtagtcttgggatggtggtagtcagaggcagggactgaagaggtgagggaatgacagaaccgacatttaccaggcaccagctgtatacattacacatgccatctcctttaatctgcatcacaaccctgtgagatcagtgctattcttagacccatttcacaggtgagcgaactgaggcctttaaaaggttacatcaacctctcaagatcagacaccaaaccatagttcagctaggtgtcgcaggggggaatacttattaagtgctaagcactgtatatgtattggttcacttaatcctcaacaaccctatgaggtagctcctgtttagagaccccctttttttagaggaggaaactaaggcttagagtgcaagagggaggtcctttgcgcaaaggcatggaggagatttgaatttaggtttagggntgggccaggaagggcacggcagccgttaaaaaaagaggcccccctgggaggaggggagctgaaagccctctccaacacccaccccaatcctggattcagacacagacatttctgtgacatccctaacttcccacctgctacctcaggccacagcacccaggcactagggctcccctaggcaggtttttgaggcatgtattatttttgcaacacggacatacatgtacctcctcctggtactgcctggggctgctgcaataagttaccctttccccattctcatctgtatgtgaagttccctggcaaggccaaagcccagggcatcagaatgagcttcctgaacaccacatccaggcatagaagagttgtgtcatacatagctcaaggttacccagaacagcaggagatgtggtccagcatttgggccttgagatccccccattcatcctcttgattgtcc

SEQ ID NO: 158 - DNA, 1021 bp, Homo sapiens
  misc_feature (561)..(561): n can be g or t.
ccaccaccgaggccgagctgctggtgtcgggcgacgagaactgcgcctacttcgaggtgtcggccaagaagaacaccaacgtggacgagatgttctacgtgctcttcagcatggccaagctgccacacgagatgagccccgccctgcatcgcaagatctccgtgcagtacggtgacgccttccaccccaggcccttctgcatgcgccgcgtcaaggagatggacgcctatggcatggtctcgcccttcgcccgccgccccagcgtcaacagtgacctcaagtacatcaaggccaaggtccttcgggaaggccaggcccgtgagagggacaagtgcaccatccagtgagcgagggatgctggggcggggcttggccagtgccttcagggaggtggccccagatgcccactgtgcgcatctccccaccgaggccccggcagcagtcttgttcacagaccttaggcaccagactggaggcccccgggcgctggcctccgcacattcgtctgccttctcacagctttcctgagtccgcttgtccacagctccttggtggtttcanctcctctgtgggaggacacatctctgcagcctcaagagttaggcagagactcaagttacaccttcctctcctggggttggaagaaatgttgatgccagaggggtgaggattgctgcgtcatatggagcctcctgggacaagcctcaggatgaaaaggacacagaaggccagatgagaaaggtctcctctctcctggcataacacccagcttggtttgggtggcagctgggagaacttctctcccagccctgcaactcttacgctctggttcagctgcctctgcaccccctcccacccccagcacacacacaagttggcccccagctgcgcctgacattgagccagtggactctgtgtctgaagggggcgtggccacacctcctagaccacgcccaccacttagaccacgcccacctcctgaccgcgttcctcagcctcctctcctaggtccctccgcccg

SEQ ID NO: 159 - DNA, 1021 bp, Homo sapiens
  misc_feature (561)..(561): n can be c or g.
gcctatggtgcagggctggcagaggcggggccaggattctagcttccccacacaccagccctgtggcatcattcttcccaacgtccaaacgtttttccaagggggagaaatggactgggtcatgtaaagaaatactcatttttagggctttttatgtggccttcaaagcacgttgcaaacaaatccctttcactcctcagaggaggagccattaggaaggtagggggcgacaggcacagcctacagcctctcctcaggaggacagagggggtcatcgcatttgagccccctgcagtcatctcgggggctcctgagggtccaggtccacatgttcgagggtctgcagcacatccacggcgctgtaggacttccaggcctgcatgttacagctcttcaggatggctcccagctgcctgccagggcctacttcgaaagtttgggggaaccccctgccctttttcctttcgtatatggcatgcatcgtctgctcccacttcactggggagaccagctgctgggccagcagcttgtggatgtgcccgggatgcctgtatctatgcncgtggacgttggagtagacagaaaccagaggcttcttaatgtcgactgcctttaaagcttgcgtcaggggctccacggctggctccatgaggcgggtgtggaatgcgccactaaccggcaacatcctggtgcgtctgaaatgaaacttagaggaattcttctggagaaaccgtagagcctggggaaggaaggaggtttcagccgagcaatgtcccagaaatccgcctttacagatctgaccattcacagggccaaactgggagggtgaccacaaagagacccacagctgctagatgtggacatgtgacctgtctgtcccagcaccatccccaggcaattcacttaacatcctggaatctcttctgtcccagccttcaaataagcacagttccatctacttcacaacgctgccaggaagagcaaaccctacaaggcatgcaacagtgtctggtagaggaaa

SEQ ID NO: 160 - DNA, 1021 bp, Homo sapiens
  misc_feature (561)..(561): n can be c or g.
acgttatcaggcacaaaccccctccagacacctgagcctcccccacaggctcccagtgaggagccatcacatgcccaggccagccgaggggccctcaggcatggggatctgggcaatggcagcaagctgggcggggggtgcagccaggatgacagcagatctgcagggcggggtcctcgccccgggccacctggctggggccgaaggtcacagctgcgtctaactgggccttgagcagctgaagctgtttcagggcttgcagcacctctggggtggccccggccacaccccccagcaggttgtagttctcaccagggtccttggacaggtcatagagcagcgggggctcatgagcagtcagagagctggaggcgtggcaggcagggtctgcagtggtatcactgtgggcagagcctggggagggggccaattctgtgcacagggcaagggcgagaggaggggccagggatctagggctccggggaggggtcagcaggtcggggggagggatccacggggaggggttaccctgggtgaagaagtgagccttgtactttccantccgcacagcaaaaaccccacggacctcgtctgggtaggacgggtagaagaagagagactgccgagggctctgggggcagagtcaggggtcacggggcggggcaggccccaagcactgcacatacctggggctgccagccctggtgggaggccctggacgtgcaccgcttcttgcccacccaggaacctgagaggtggcgccacttggatgccactcagtgcaggaggcactgaggcacagactctcaggcactgcccacactcaccccaggggaaggccaggacaggggccaaggatctgggatcaggggtcaccggccctaccttgcctgtgcccagcagcagggggctgaggtcaaagccatccaaggtgacattgggcagtggggccccagccagggctgccagggtaggcagcaggtccagggagctggccagctcgtgggtcacgcctgggggc

SEQ ID NO: 161 - DNA, 1021 bp, Homo sapiens
  misc_feature (561)..(561): n can be a or g.
gaatggtaagaaacattcttcagctcaagatggtgaccagaggcatccagcactcacttccttcacaaaggactcaaacagcaaatgaataatcacatgtcaagtagagcagcttagaaagaacactggaattcagagggaaaggacaaggaacttcggaaacatgcaaagagaatgatgtgaagcagccggcccagccaggatcagctcagatccaagagaaactgcccaacgtagggaaaaggtaaatgagagatccccgcaaggctgcattcccaccacagactcctgtggccctagccacagagagccccttggccctcatgggctttgagactagtatagagagccgcctgcattgttccaaagagggattttatgatgggtcctacacatcctctgagacctgagcagctgcagcacagcaccattttgagagcccacccctgaccagaccccatcccgccctggggctcaacagcccctgcatctccacatccatggagtcctgctgacattccgccatgtccacccagaaggctgcagcctcacaatgcaggntgactgggtccccagcaatctagtctacacatgtcctataacctgggaatgggtggtgcaccacaccagggaggctgcccctgggacaaagggagccaaagcccatgtttcccagagccgcagagctgcccgcctgggaccactgccactgacagcacccccaccatccccccagcagcggggtcactgtgcacttgtgatatggtttggctgtgtccccacccaaatctcatctccagttgtaatccaaattgtaatccccacgtgtcaggggagggacctggtgggaggtcattggattacaggggcggtttcctccatgttgttctcatgatagtgagtaaattctcatgagatctgatggttttataagtgtttgatagttccttcttcacacacactctctcctgtcgccatgtgaaaatgtccttgcttcccctttgccttccgccatgactgt

SEQ ID NO: 162 - DNA, 1021 bp, Homo sapiens
  misc_feature (508)..(508): n can be g or t.
cacctcccttaactccccagccatgccccgtgggtatctgttttcccagttttgtagatgaaagcacagctcagagaggtttactcagttgcctggagtcacacagtcaacaagtggagagccagtcattgaatctggtaccacaaactcttcctgctgcaacagctgtgcttttgcaggcactgactttggaataccctcagctgattcacagggtcctttgtcctggggaatggccttccctgtctccttcagggaaagggtttcatccttcagggaagattcattgaatcaggatttgctgggtttttttcatttttttttttcatttcttttttttttacacgaatgggcttcctggcccgcattttgatttgcgcttgggtttatgaattgaggaatcacagtcagccttgggaattagttgcaagataaatattgcaatcctggttaaggacttaagaattgtcacttgtgtgtgtatattgttgttgttgttgcaacggtnctgtgtacgcacggttacagtggatcaaatttggggagttaggaagtggcgttggtttgtggttagacttgggggaggtgtcgctttcggttgttggtgtgctggtggctgtgttcctgtgatatggaatgtactgtctgagaatgtgttcaggggtctgtggttatgtggatatgggtgtgtagctgctgatgacatggatggagggatgtatctgggtgtgtttctgcagaacaagtgatacctgtaccatgtgactttgtcagttccaccatgtccaggcacaggtcgggggggttgtccatggttctgaacgtatctgcccccattttacagataggaaaccaagacttagagaggccaagtcatctgcttgaagtcatctagctgagaagcggctgagcctgaagggaaaccagggctgccttcagagtccagcctcttttccctgctccccaggaaaggttttagtaacaataaaaggtttaaatgccagcaaaaggtctaaacgcc

SEQ ID NO: 163 - DNA, 1021 bp, Homo sapiens
  misc_feature (561)..(561): n can be c or t.
ttgccccacagacaagatgatcccccctggcatgttgttaggggcaaattgctgtcctgctcagagtggcatctttcaatgttgcctccatcttggccaagaggtccctgcctcctgatccggcacagctgagctgaggcagatgtgaccagttttcaagctaccagccctgggcagaggaagatgtcaacaattccagagcagagggaagaggcaccttccttgaccacaccagtggcctcctgaagttccatgcttttaagagctgggaccttgggaggatgattcaaaccctcaattcctcctccctgggaactttttaccacctttacctatttatcaaaatcatattcatctttaccatcactgtcactgtaatctacattccatcacctttatcaggtgctgctgagtacaaagcacttgggatgggagacacagcactgaattcacaaacattggaccaaactgtttgtccccatctgggttcatgaggccacctctttgctcaatccatgcctcttgccctcagtcaacaagacattcctagagggaaagggntgctgctctgggagtcaacctgagttcctccctcctgggaagctgggttggcaagattctaggacactcacctgcatggacatcacctctgtgacaaatgcttacctgtttctcatcttcagacttggcgatatcaagcctgttctggaccatgaccaggctggctcatatctctggtttagagaaacctatgaataactggggacaaacagactctttggtagcagcagacacatgtgatccatcaagatcaaccaaggttgcaactggagcgtccactgccagagacctttggctcttcaagctcgggacaaaaaagaagactctgttgtcccttggtaacccagtccctgcttttgtagctatcacagcagaaagcaactcttcctgaagaccaaacactcgtcatccacattccttgaatggccaatccttccatctggaggcctggctcagaaag

SEQ ID NO: 164 - DNA, 1021 bp, Homo sapiens
  misc_feature (561)..(561): n can be c or t.
gtttgatgggacaagataggacagtggttaagagtgtgacctcagcagctgactgcctgggtgtaaagcctaccatgtggtcaagcacacgggtggctctaccacttaccaaccatgtgaccttgggcggttaacagccctgtgactcggtttccccatctgaaaagtgaggatcatagcagtatctacctcctgcggtggtcggaaggcagaaaagaattggcacatgtgaaagtacttagcacaggcttggtgcatagcaagtcctgaggaaatgtattcactgtcatcagtttcacccgctttgaaaggcaggcaaagaaagcacctgacaaaaccttttgatcccccacgccttgtctcccacacccaggacattcccctgactcccatcttcacggacaccgtgatattccggattgctccgtggatcatgacccccaacatcctgcctcccgtgtcggtgtttgtgtgctggtaaggggtgaccccagcctggagaggcagcgtggcagagtggccaagggccgagtcagatggacatgagtctagttcctggcnccgtcacttaccactgtgttaccttgagcaactctcttggcctctctgaaatgcccacatcgtagagtcactgtgagaattaaatgagatgaagcaggcaaagcatttatccaaggcccagcacacagggtatgctctaaaaataatagctgccattctgttctcttgcttaaccctctaccaggcagttagcaacctcctatgcagtggaaatgcagctcatctgactcattcattaaacagacttttattgaccacctattatgagctaggtccacaacagcaagatgagaaccaagggaaaaagtgcctgtgattagatggctagcaacccaaaagggacccttggggtcctcacgtccatcccatcttcatgccaggcagagctcttctttgaaaatctgtggagtcagaggtgtaaggcattgggacaggtgggggtgagagttccccccctcat

SEQ ID NO: 165 - DNA, 1021 bp, Homo sapiens
  misc_feature (561)..(561): n can be c or t.
gccctgccctgtagtggcttctcaatgaatatgtagttgccttattctcacaaacaccaggctttcctcacatcagcacccggtgtgataggtaagagtgtgtgatactagaaacgtcagcttatccaaaaatgtatttctttctctcatgagagcctcgtgagctctccagcttgctggaactttctaagacctaacacttgccaaattccttgcagcaattgtctggtttgtggtaccacaatcgaacccaccaccctgacgtatttgctgctcagaaccaccgatcttccaagttctcatcactccagtgcagctcctgtgacaaaaccttccccaacaccattgagcacaagaagcacatcaaagcagaacatgcaggtggagtttgggtaccgccggcagagagcgggaggggcttgatggtgtagcctcctgggccccaccagaaatccccacttctaatagtctagtgtgatgtgcagtggtcattgcctttgttctgccccagcgcacctgtccgtagcagcagcagtcagtagcagcagcttgagtggcagnggttctcaaacctggaagcgtagcgcagtgtaagctcccaccagccctgagtgagagcttgttggggcacctgggaagggtgtcagcctcagtggtaggcaggcctgagtggaaatcctgattccagcacttatcagctacatgaccttggcaagtgacttcccttttctgagcctgtttccttctctccaggatggcagttattaaaacctactttgcaggtaaatttggtgataatcacaacagctgtcagttacagagtgtttcctatgtgcaagacaccatgctaagcacctcgtgtatattttctcatttcattctcacaacatccctctgagcatccaggcagtctggatccagatctcatgctctttaccactagattgtacaaatataccataggttataagattcctggcacttggtagatgcttgctaagtattggccatcgccccaacc

SEQ ID NO: 166 - DNA, 1021 bp, Homo sapiens
  misc_feature (561)..(561): n can be a or g.
taaatttcttacaaggtctcttctagttctaattttttaaaaaatgttatgacctctgcccagattttttgtctcactggaattttatgaaatcaaatagtttgtaagtggaccattataggactgttttgcccagttctttgttgtaagggtgtttgaccggttgaatcatggtatttaaaaaattcttatacaactccagatctaatggtaggctaagttgtggtgatgcttatactcagtgatattgggtgtgtattataagaatgaagagagcggagaacaaacataaacattaatgttaatgacaaacattaacccaagtacaaggttaatgtttagtcaatatagcaaacatgtaatttacaagattaaaaataattaggcttgtgataaagtcaatgaatttcctacgtaattgtaacattagactgttttattatttgtcctgacattttgcagaatccaagattaattaaagaaatggtttcaagaagagggtgaatactataaaaatagacttaccttcctgaattgaggaattcatcaggaaagcctcangtgtgcaaatgagccatccttccagagggaaatttcttagaattatcccacgatttgagccaaagcacttccgatagaatttttaacctctagttggttctgctccttccatttttactaatttttaagaaaatactatgacttataattgtatctggaatgattatcaactccttttcatccactgacttaaatttgattataaatatgctttacataaagatctagaccttataatttgaattcaagtgaattgttgtgactagcatgtaaattattattatggattgtaaatcttaacataggtagttctgtgcccttaaattgataaaccagttatctcttgtaatcatgtgtactaagatatacgtagtaaagtgattgtatcagtttttatcataagcagtcatagttcagatagttcagaagtttagtgtctgctgtttctattaggaaagtg

SEQ ID NO: 167 - DNA, 1021 bp, Homo sapiens
  misc_feature (561)..(561): n can be c or g.
acttggtgactttctctgcaccaggtgagcccctagtctacactgcactgcaccccccccccaccccggcgcacgcacacacacacacacacacacacacacacacacacacacaggcatgcacaggccctcctgtgagagatagccctaaggagggaaccgtccctagagccggtccccagccgctcggcacttcccgcccacgcgcccggtcccacagtgcagcggaccctcactcaccccgcggatgtcccagtaccccagtgtcatggacatgatgctggttggtgtcgattctgcagacaggcctcagctgggctgaactgcgacctcctctggggttcccggcacgcaggggctggacctagcgccagacccgccccctcggccccgctgcgcccgccgatcttcaaggtcgtcacttccaaccggccgatcttcaaggtcgtcacttccaaccaacaggcgcgggaggcacggagcaggttgctggatcctcactggctggaaggagtaagatccaccgccacctccgagtgttcagggagcaaggtccggaancactaggaggggctcggcctcgccagcttccgtagccccgccccgccccgctccgcttcggacctctgctgggtccccagggactcggctgtgcgcgtgagagtaaagccagatcgtaagagaaaagttcttcccccgtttcttcttctccggacgtcgcccagccttctgcctctcggctgccgagttcccacaggctctgggagactgaggctgccagggtcagactaaagagaggtctcagagagtttaattcaacacttcttggctactaagtcttagaagtctgatggtgtgctctctctgctgagttggggagcgtgaatggaggctatgtcaccgaagctgatagagctcagtctctgttgcagatgctcccgacccttttgcattgggccagttccccagctctgagactgggtccaggctcaggaagtggcctatgtgtcaaggtggattc

SEQ ID NO: 168 - DNA, 1021 bp, Homo sapiens
  misc_feature (561)..(561): n can be g or a.
gaatggtcatttttgatgttttgttgttgttgctattttcgttgttgaggataactataattttttgtgccaaaaatgtggcaaacctttctatggggaaaacgatagaaatggcacttaaccctaacccattggacataatctattatctgtttttactaaaatccactgaacctgtagaaatcttagattaatcagaaacacactcttttcttgtgcttctcaataaataattgaattgtttttgcccaggaattacccctgagcaactaaaatgtttaccttcctgcagttataaaaatctcggtgggggttgtttttcagctcctttaactcgtccatctcgttaagcatctgatggacctggaacttggaggagaggaacttcaggcgccggtgggtataggtcttactgtgaaaaataaaatcacataattccaaaaagtttcaggcattcaagaaaaacagtcacaatttcaaaactatcaggacctttatcattcataggaaataattgttggaacaaaccttttagtttactctgcagttaatcccactganaagtagtgggctccaaaggcttaatcttttcaataatgttggacataagaatgagggagaacttggaaaggtatcttaaaactcaatggagagagtgttattcaaagtttggggtcagcagattcgagtgtgaatcctggctcagccagctgtgtcactttaggcaagttacttaagtcatcaaagtctcagctcataaaactggaattatgaaaataaccacctcacagtgaaaagtgtaagcaataaaaggaacaatgtgcatgaagggcttaatacagtgtttgaacatagtaagcatttagtaaatacttagtctcactatcagtagaagtagtactagttgttgtttaggtcttgtagtactagttgttgttgtttaggtctcactaaacacttacacaggtccttgagcaattaaagcaagtaaaaaattcatatcgtctaagaaggtgtccag

SEQ ID NO: 169 - DNA, 1021 bp, Homo sapiens
  misc_feature (561)..(561): n can be t or c.
gaaagctgagaaagaggcacaccaagactaagggaaagaggccgggaagggtaaaaaggtgaaatgaaaagaggttggtgaatgactaagaacggttggataggacaaataagttccaatgttcgatagcagacgagggtgactacagttagcaatatattgtatatttcaaagtagctagaagacttaaaatgttatcaacacatagaaatgaaatatacctaaggtgatgtatccttcaaatacccggacttgatcattacacattccgggcatgtaaaaaacgcttccatgtaccccatttcataaatatgtaaaatattatgtatcattaaaagaaagaacaaaaaagacagggaaaatgcatatgctgtgctccactcagccaacaaacttctgctctaagcagggatattgattccaaaggctagcttgcgtttcttaaaaataattaaaaacaacaacatgtcatttatttcagagctggaggctagaaataaattactcaaatctcgcaactatgtaaactatgaaaatgaaacaagctagttaccttttatngttcagtttaaaaaagttcttcttctttgctcctccattgcggtccccttcaagatccattccgacctgaagagaaaccgcagctcattagccaaatgcatgagcctcaggcgcgctggaggtgagactaacctctagtcccccgtcgaagccagagagcagtaagagggagcgcccgccgttgatgccccagctgctctggccgcgatgggcactgcaggggctttcctgtgcgcggggtctccagcatctccacgaaggcagagttgggggtctggcagcgcgttctggactttgcccgccgccagtgcgattctccctcccggttccagtcgccgcggacgatgcttcctcccacccaccgcccgcgggctcagagagcaggtccccgcaccgcgcgggctgtgcgcgctccgggcaacatggtccagtgccactacggtttgggcgctgctccagg

SEQ ID NO: 170 - DNA, 1021 bp, Homo sapiens
  misc_feature (561)..(561): n can be g or a.
gtgagttttgaggcttgggagagagctgcaaggaggaagaaggaagagaaataggggagagacatggggagagacagtcatgcctacttcctcagcaggccagaagcagcatgtgcaggtggggacccagactctgtacttggacttaaagtgaaaggctttccagatattgtacttacccctaaggctgacaaaggtggagcctcaagcctatagctttggatcaagacaattgttccagttctcctatcccagaaatgttcctctctcctaaacctgaagtggtcgaacactttcatcccttcctcacaaggagggtcaggtgatcaggtaaaggtaacaactaacccaaacaggaagtgtggccagatgcttgtatacaggtaagggtgtgatttggttgctaatttctcttcacttctgggagaccagccccttataaatcaaactataggccagagaggctgccacatgctcccaggctgtttatttgaagagagacttacattaggcagtgactcgatgaaggcatgtatgttggcctcctttgctgccctcacnatctcttcctgtgacaccacccggctgttgtctccataggcaatgttctcagcaatgctgcagtcaaacaggatgggctcctgggacacgatgcccaggtgtgctcggagccactgaacattcagtcgctttatttctttgccatcaagcagctgaaaacaagagttcacagatcaacttcaggaccagcacactttgaatgtagcacaattaacatcattatttcttacactgaaactgccaagttactgtgagattaaggaaaagtttgtgtgattaaaatttggatagtgaaggttaacccaacaaggtcataattgtatgccttgaggaactgtcatgtttcctgtgtttcaaccatggtttctgatgtatgcatgtggtaggcagaataatgttccctctcccacaagacatctgtgtcctaatccctggatcctgtgaatgtgttatgttacat

SEQ ID NO: 171 - DNA, 1021 bp, Homo sapiens
  misc_feature (561)..(561): n can be g or a.
gccttgccttcccccaggcaggtttgagaggtctgggtctcaactgactggggcagcaggacctcatcccctccctgccctacacccagcctgccccagccctgcagtctgttgttccttagtcagggaggagcccaaaagtgtgaccaaaccaagggaacactcaacttctggcttcctccctctttgggtagccctcaagccactggactttgaagtcagcaggtaattctccaaatggaagaacttttttttttttttttaaaagcagagccaaggaagccacattttgagtgatgtggtttttgaagaaaaaagaaaaagagatcccagataaaaatgatcttatgtgaagggagtaaatggatgcacagaaacagcagcagctcccgagccacctggtggagcacaggggccctccctggcctcccccaacactggggctggggtctgggggctgcccagcagggtgatgtggctcccttgggcctgagagcaccctggagggagttgaccctggggggcaatgttcccaggacgcagtacctgatatccaagtcngtcgctgtctcccgctctgggctgcagcaggggaggaaaggcatactgagctctcatgggagtgaaccatatcctccaggaagatcctgagctccctccaacccaacatgagcatgcctttacaatcccctggacccagtctgtagccacaaatgctgcatagagaggtgtggagagtggggtgtgcccatcttggggaagcctctgctgcctgaccacgtgggtgtgtgaggagggccctggaggacccagttaagagggagaatggggagaggtgccattggtgcaggctctggggggaaaacttgtcagatcaggagtatgaagcccgcaatgtggctcctccagacccagcctctgcattcaggttggaatgaataggctgaggtctgaggctgatacagctgcacaaacagctggggcaaggagtgctctggacagagccaggccaggccaggca

SEQ ID NO: 172 - DNA, 1021 bp, Homo sapiens
  misc_feature (561)..(561): n can be t or c.
tgtcaggcaagattcaaatcaaaataattaattttaaatgacatgcatactttttggagagaaaagtttgggttacaattagccaatctgttaaaactcaaagaaatctaatccaaacgtaatacacatgtctgtaccattttttttagcctattctctcttcagacttatacttaatcacaaataacattcttctttctattaattaattccaaaaactggctcacagccatatatgacagtcatttattgctactagggacataaaatttctaaataatcagaaatccacgttgtcatttatgaatattctctctccttgcaaaccaaaaaaatcatctttaaccttacctgatagattttggcatccctcattagtttttctacaggatattctgtattaaatccattgcctccaagtatctgcacagcatcagtagctaactgatttgcaatatctccagcaaatgcctttgcaatagaagcataataggtatttcgacgaccagaatcaacctcccaagctgctctctggtaactcattctagctagttcaacttncattgccatttcagccagcataaatgatattgcttggtgctagaattaaaaagaaaaaaattaaaggatatttattgagaaaacttaaaagttttttcctggggctttttcatttttatagtgacggggtcttgctatgttgcccaggctggtctgcaactcctggcctcaagcaatcctcctacttaggcctctcaaagtgctgagattacaggcgtgagccactgtgcctgacctttttattttttaaacttttcattaacgaattttaggtttatagaagttacacccagcttcctctaatgttaacatattaccaaaccatagtgccatgatcgagaacaggacattaacactggtatagtattaacaactaaactataagccttactcaaatctggtcaagttttctactaatgttctttttccaccattatacgttgaatttagttatttcttc

SEQ ID NO: 173 - DNA, 1021 bp, Homo sapiens
  misc_feature (561)..(561): n can be a or g.
aggtagcggccacagaagagccaaaagctcccgggttggctggtaaggacaccacctccagctttagccctctggggccagccagggtagccgggaagcagtggtggcccgccctccagggagcagttgggccccgcccgggccagccccaggagaaggagggcgaggggaggggagggaaaggggaggagtgcctcgccccttcgcggctgccggcgtgccattggccgaaagttcccgtacgtcacggcgagggcagttcccctaaagtcctgtgcacataacgggcagaacgcactgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatngaagccatgcgccggaccacgacgtcacgcaggaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccat

SEQ ID NO: 174 - DNA, 1021 bp, Homo sapiens
  misc_feature (561)..(561): n can be c or t.
cagagccccgccgtgggtccgcccgctgaggcgcccccagccagtgcgctcacctgccagactgcgcgccatggggcaacccgggaacggcagcgccttcttgctggcacccaatagaagccatgcgccggaccacgacgtcacgcaggaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttanctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtc

SEQ ID NO: 175 - DNA, 1021 bp, Homo sapiens
  misc_feature (561)..(561): n can be c or t.
ccactccggagcacctggctctgccctcaggaactccctgagctttgcacacagggccgagacacctggatttctctggttccctgagtggggccagcttggaagaatttcccaaagcctattagagcaacggctgcctcctgcctgcctccttgggctgggcagggctgagggcggagggagagagagagagagggagggggagaggaggaaggaaaaagttggcaggccgacagcacagccgtgtctgcatccatccagaggaggtctgtgtggtgtggggcgggccaggagcgaagagaggccttcctccctttgtgctccccccgccccccggccctataaataggcccagcccaggctgtggctcagctctcagagggaattgagcacccggcagcggtctcaggccaagccccctgccagcatggccagcgagttcaagaagaagctcttctggagggcagtggtggccgagttcctggccacgaccctctttgtcttcatcagcatcggttctgccctgggcttcaaatacccggtggggaacaaccagacggnggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacgtgagtggggtgtccctgggcttgggggggttctagaatgatgctgaaaggcactggttccatcctctgcccattgtgcagatggggacactgaggaacggagaggacaagaggttgctggaggtcacgtagagagctggggggaagagctggggctggaactcagctatgcatgcctcccaaagcctgttttctgccaggcactgtggg

SEQ ID NO: 176 - DNA, 1021 bp, Homo sapiens
  misc_feature (561)..(561): n can be a or c.
ctcctcaccagtcctcaccacctctctcccctgcagctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctgtgagtcaggggccctcccagatggaggtgggggaagggagggcgggggctggtggggtgccctgccatgggcagccagtgggactcccgacagggctcttgccattgggtggaggatggcgggtcagcgctgggggctgggggcagggtcctgccctggagaggagcacagggacctcctgcccagcttggggtcagcactcctctttccctgggtctcattgtcccccaccctgattgttctctttctccctccaacctctccctcctctcactctctcttcacctatgactctctgccttcgcccctccctctgtttctttccctcacagattgactacactggctgtgggattaancctgctcggtcctttggctccgcggtgatcacacacaacttcagcaaccactgggtaggagacccacggggggtggggtgggaagctttggtgtcccatggtaagcctgaccccaccctcacagtgtcccttcctgttctggaggctctgggagacagccagaggacaggaaatcaggaaactgaggcctgccatgtagaggcaggctgggggtcacactgccagcactttcaggcctagtctctgccctcccagctcggccctgccccatgctgcctggcctccaggtcttcccagctgcgtggttaaaagtggggctccaaatcctggctcagccactttcgggtttagcatgaccttgcgcagtgtgcttgagctttggtttcctgagctgcggagggggatatggtggtgcccacctctcagggtggccgagaagaggaaagggctcactccccat

SEQ ID NO: 177 - DNA, 1021 bp, Homo sapiens
  misc_feature (561)..(561): n can be c or t.
tttgactccctgtacctttaagagggacccttaaatttaaaaatctattgtattttttttttagtaggggtagggaatatttagggaatttggaaggggttatatagttctttaagaatcaaatagcacatcttcctgaaaatagcacgtagacaaagtttttttggagataaccttaggaatatcgtaactctctgatgccacctccatatgtgatcctatgttgattataagattttgatcagtggctttcagacttttttgactgcaacctagaataaaagattcatttacattgtgacctagaacacacacacacacacacactctctctccgccactctcctgcacacagaaatcattgatgcttacaacaattcttactcttactatgggtgatttactttgatatgctctgttttttttttcatttacaaaactgtggattaattttttttgacatgctaaattgatctcagtaatagattgtatttattcttccttagattcttctttggagcagaataaaagatctggcccatcagttcacacaggtccagngggacatgttcaccctggaggacacgctgctaggctaccttgctgatgacctcacatggtgtggtgaattcaacacttccagtgaggctctgggccctgtgggattgcccagggatgtggagggtgaacagagtgacttctgctggaggccctgaatgattagtgtggaggacagagccacaggcacccatcctgatgccatctatacttatattagtccatttgtgttgctattaaggaatacctgaggctgcgtaatttataaagaaaagaggtttatttgactcacagttacgcaggctgtacaagaagtagggtaccagcatccacttcgggtgaaggcctgaggctgtttccactcatggagaaggggaaggggagctggcatttacagagatcacatggtgagggaggaaagcaaggagaggtcaggggaggtgccaggctgtttgtaatgacc

SEQ ID NO: 178 - DNA, 1021 bp, Homo sapiens
  misc_feature (562)..(562): n can be a or g.
tcaaattatcatcgcttttttatttcaggattacaccaaagactgtttccaacttgactgaggtaggtagtcttggatagactgggggaaataagtcctgtgggacctcctgccttaaagaaagcaggcggagggccctaaaggaaatcaggcaaccagaccaaaagaatgtggaccaggtggtccatgctgtgtctcttgtgacccttcttctccctgccatgtcttttgggagagcccttgtgttgcaaaaatgagagtgtggtggtatggattggggtttaggcagaacagtactggccaagcagcgcctccctggacctcaattttccctctgtggaatgggctagcaatcctgggcctccccagggcgaaggaaagaccactcaggaagggcaccgtctggggcaggaaaacggagtgggttggatgtatttttttcacggatgggcatgaggatgaatgcttgtccaggccgtgcagcatctgccttgtgggtcacttctgtgctccagggaggactcaccatgggcatttgattggcagagcagctccgagtccntccagagcttcctgcagtcaatgatcaccgctgtgggcatccctgaggtcatgtctcgtaagtgtgggctggaggggaaactgggtgccgaggctgacagagcttcccatttcaccttgtgggcccttcccaggcagagcttcaggtgcccctcttcccagtcattgatacttagcggtcctggccccctttcctctccctgctggtggtattgcacgccaatgactcggccagatgcccagacccctgttcttggtttacctgcagaatattatctttgccaccccgcgggatggctcaacccactttcaggatgcaggtctcctaatagcaacctgatatagcagaaagacccctgggctgggagtctgagacctagttctagcccagccctgaacctcagtttccctttctgtgaaacaagaatgttgaacttgatgattcccaattttccttttg

SEQ ID NO: 179 - DNA, 1021 bp, Homo sapiens
  misc_feature (561)..(561): n can be g or a.
gggccaaggccacaaagtctcaggacaaggcagactgcagacccaggggacgtgcgcggaccggggcttgtttcggtcctgggtgttctcagccttgatgtggacactagcggctctggtgcacttgctcggaggaagcagccacgtgtgggtgtcctggcctcagccggcagtaaccagcagacacacagcacggaaccctccaccctaccaggaagcccaggcaagaccccccagcagtgcatgctgaccccagaccctggcgacggatcggagctcctcggatttggagtggatccttacaaatcctgcacactagacagcagacacaggccctgccagagccagggacccgaatttttgtttggaaaaacactgaggtaagtggggggtggctcctgtccaggcagcccggccggtgggacagtggggagggtcggctccaagccctcctgagccctagagggggtgcgggacggggactcacaggagatgcaggacggcccgaacatagtaattcctggtaaagggcccgaacagcttcaccacggcggtcatgtncttctgtcccctgggggagggaggaaggcgagacggcgcggctgggcctctcccactcgggactcctttgctgccctgctgaccaccccagggcacccaggcctctttcctcccacaaaacacaccgggcaggcaccggccttggtttacccacaagcaccaaagggttggttccggagcctccaagtgagaaaccaagctccacccaaccctgtgagccctgcctgggccccgcagcccccggagagaccccagagcaggaggagactcaccagcgctccatggtggagcccttcttcctcttcccccgggggtactccagcaggcacacaaacacgcccgccacactgaagccatgtggttaaggaacagcccagctcagcctgaggggccacagggaactccctttactgaagacaacacagagaggggcccgagcacggtggctcatgcctggaat

SEQ ID NO: 180 - DNA, 1021 bp, Homo sapiens
  misc_feature (561)..(561): n can be a or t.
taatacatgaaagaagaagctagtcaatgtggagctctattgtgtcccgggatcaacaaagacaagatatctttaaaatcgtcttctaaatttaccctaatgtaaaacaaatccaataaaactctaatgtaattttttaagaatttaaatttggaataattccaaagaacaatttttcttaattttctacagccagaatatatacctttaaaaaaaatgaaaacagagattaactttctcagaattggttgactcactctttccttttatttttcttccatggaattttccagttaacttgagaaagtggaatcgaattccgatgttgaattttccttctggccccattcatgtggcaggtggtgattcaggtactactgggggctgctcagacaaacctcctcatcagacatcaagaggctgttgcaccaggagggccggtaccgtgtctagaggtggtcggcatggggttggagttgtattacataaaccctactccaaacaaatgcatggggatgtggctggagttccccgttgtctaaccagtgccaaagggcaggncggtacctcaccccacgttcttaactatgggttggcaacatgttcctggatgtgtttgctggcacagtgacaggtgctagcaaccagggtgttgacacagtccaactccatcctcaccaggtcactggctggaacccctgggggccaccattgcgggaatcagcctttgaaacgatggccaacagcagctaataataaaccagtaatttgggatagacgagtagcaagagggcattggttggtgggtcaccctccttctcagaacacattataaaaaccttccgtttccacaggattgtctcccgggctggcagcagggccccagcggcaccatgtctgccctcggagtcaccgtggccctgctggtgtgggcggccttcctcctgctggtgtccatgtggaggcaggtgcacagcagctggaatctgcccccaggccctttcccgcttcccatcatcgg

SEQ ID NO: 181 - DNA, 1021 bp, Homo sapiens
  misc_feature (561)..(561): n can be g or a.
aaaaagaggaattaaattgtgtagatgcctttaaagaacatttttctagcatctttctacatctttccctaagtggcctcttgagcccagtcggattttggttatatgccatgatagtaatcataagaatcagttaaaaatgatccaaaaatgcacgaatacagtcgattccctctcatttattccttgtggaaaaagaaaaacacaaatcttaaaaactaaagcaagtcagggaagcctggaaagatacccagatttgataacatgttagaaggaaatccaggctaaggaatctcattttctagctttgatctggttgtcagttgggatggacttgcccaagtgatggcccacagaaaggccaaatttcttgtttttctcctcatcctgtacctcttttttcattaagaatcctgcctggaagtttaggtcaaagaggctgcttggagcaaaatacagtggtgtctcatcccaaatattctccaggcgtttcttccatccttccaggatttgaattcgggcgtctgctggagtgtgcccaatgctatatgtcagttgagnttctaagacttggaagccacagaaatgcagaatgccactctgaggatacagaaagcacagagaggtaagtcaaccaattccatgcagttgtactataaacaacagaagttggtctgggcttctcagtaagacactctgataaggaggcctcaggcacactagagaatcagttcagagctagcgtctctctcttaccctctacctagccgttaccaattttagccttctcaggtgtgttcttctttaaatgcataaaccttgaaactgtgccaacctggatcctttgccaagaaggctggaagttctgttactttagggagtctcagtttcttggcaggtgactcaccaagacctgcgtgggtgcatttctctgcctctccatataactagatgagtcctttttttctttttctttttttttttttttttgaggcagagtctcgctcggtcgtccaggctg

SEQ ID NO: 182 - DNA, 1021 bp, Homo sapiens
  misc_feature (561)..(561): n can be g or a.
ctcatgtaggaaccagcaggctactagaaattaaagtttaaatctgggagaaggttagcgttaagtgtgtgtatacgagagtcaagccagagaggagggcagtaatgctgtggggttgcatgaaattcaccaaaggagagcatgcaaagtgaagagggaggcaaatgaagatggagccccaaggagcacttacatttaaaaatatgggcagaggaagaggaatcgtcaaaggagactaaaaagtagccaaggcagggagcatttcaagaaggagagaaagatccactttgccatatgctgcagaaagagtccaacaggttgagaaatgacagtactcgtgattccaaaggtaatgaaaaaaatccccagaattctatgcatgaattaattacgtgattaaacatacaaatgtactgttctccaagaaaactgagctgtttccatattcagcattgaataccaagatattattttcttgtttgtagagatattcatgatctaaagagagaaaacacccagatcaaaatttcaagttgttattaaacatcttcataagctganaattacagaatacagtttaagctcacaaataccaaataggcatttctaagttgagaaaacatgaatgatattatactaacattcattcattttttcatcattattgtcaaggtttcaattcacatttaattttttattatacatgtcaaagaaatacttgggttcctttcagtctttctccctttgcacttcaagtagaaaaagaaaaaaaaaactctctatagaatttttaaaaacaaggattacctcttctcagtgccataaaagcccacatctcgacttaactagaatgaatgtaagcataaaatctgccctaccccaaaaaattcttacctgaaatccatcttaaggagtataacttcagtctataagtattttttaagtaatcagttagagtgtaagttttgcgactgtcagctgtagcatcatctgctggttgaaagaaagagccaaatgttca

SEQ ID NO: 183 - DNA, 1021 bp, Homo sapiens
  misc_feature (561)..(561): n can be t or g.
agcgttcagagaaggagcgcaggcagaagtcaccgcgggcggcggagacgcgcgtcctgcaccgctgctccgggcggtggagtcactcgccgctggcaagtttcggccccgagttaaacattagtgagcgccgagcccgctgggtataaaggcgccgcgggcaggctgcagggcaggcggcgcgggagcaggcgcgcgtggcgcggggcactggcatcccggccggggggagcccgcgagggccccctgagggcggtgtagggcgctgggcggcagccggggcgcagagtgcggggcccggaggagccgtgggggaggggaaagggcgcgcggcctcggatgcgcagaccctgggccggcgactcggggaccctgctccctcttagctaaaaatgacgtcggcgttcagctcctccaacctcacgtggacaggcgagggaaccgagacccagagagggcaggggactttggcaaactcacacagcccaccgcaggcaactggaactgaaacccaggactccgtctcttgccagtgaaagttatgttaggaagcagtgaggngtctaaagcagtatgaaaggcaaagagaaaaggtgattgttccctcttgaatggcccttggaagctgagtatctggattcaccctccctagggaatttcccgattgtcttgcaggcttacacactcatcaagatgacaaaaataatgacagtaacacttatgtggaacttgactttttcccaggtgctgctctaagcatttactgtgtttgttttacaggaaggaagactgtacacagagaataaataacttggccaagccattcagctaggaagttgtagatcctaaattaagagttcaaggtcttaatggctactctatgcggcctctcatagtcttttcaagggttttggagaagaataaaagatcaggtatggcttctccctcccccagctctctattgttccctaaaggattattcattcgttcattcattcctacatcctcccatttattccag

SEQ ID NO: 184 - DNA, 1021 bp, Homo sapiens
  misc_feature (561)..(561): n can be a or g.
ttctgatcagttttctatgttaaataaatatacatctaccttgtcagtttagatgactgtactggactccagtatactgtcaaactatacttgattaatcctgtattgctggatacgtggggctttctccctaccctccagattttaaattattgaacaagtatttatggaggcctgctgtgagccaggagctgtcctgagccctggaaacccagcagtggctgtacagacctggcccagctgtcagggggcacctctaaggaaaccgggaggcaataatcgtagctcccttgcagggaggttgtgaaggctgagtgaggacatctgtgcacctggagcacagtgtgagtgtgaaaccagtgtcagcccttattactgtcaataccatgaaggggcggcgggggcactaagggtggcaggactcaatatctaggctctggggggtgccagagcctgaccgtgcagggtcttctctctccctccaccctgactgtgctctgtccccccagggctggacatccacttcatccacgtgaagcccccccagctgcccgcaggccntaccccgaagcccttgctgatggtgcacggctggcccggctctttctacgagttttataagatcatcccactcctgactgaccccaagaaccatggcctgagcgatgagcacgtttttgaagtcatctgcccttccatccctggctatggcttctcagaggcatcctccaagaagggtacggggctgctagaggttccataactgccccgtcctcgccaagggtgggcccggtgttcccaccaggctctccttccggcggggtgagcagggagttggcccgaggaagctgggaaaggaggggcctgagaggccggccccagacacaccgccctccggggctggagatgccacccctatatttgggctccaggattccttcttgcctctgtgagcttttctgacctccacctgggggtaggcgggcctgagaaatttcatagaacaccagagggcccaagg

SEQ ID NO: 185 - DNA, 1021 bp, Homo sapiens
  misc_feature (561)..(561): n can be a or g.
tttctggatcacgttttcatatattctggttcagtacatctatctttgagttatctttaatatactgaaccagaacatacaggaatgtgatccagaacatcattggccatcagattttctagtatatgtgatgtgcacctcttataaattataattgaattcactgccatatctccaaggggtgtcactcttgtactccagaagatactggttatgcacaagaaatcatgcagggacaaatagacagataccatttagtgttttgatttattctgagggaattttaaatttgtaatatgtatcttaatcattaaatatttttcttaacccacttttcttttttcatactgtatctgccaaaaccatttgctagcatagaaaagagggatttctttctgtatttctcttagacatttgtatccagtgtaaataaacatcctgattttgcaactactggccagtgggatgttaccactgaaagggatggtaaaaaagaatcggctgtctttgatgctgtaatggtttgttccggacatcatgtgtatcccaacctaccaaaanagtcctttccaggtaaggccaaaatttaagctgctagccacataactgacaaaaatgaatatcttgataatgtcttcttttttctaaaagtataagcaggttaaattaaaatatacttctgttatatctaatatgcttggtgtgttaaaatagcacattattgtgactgcatctattcacaaggtcgcttctgttaaagtctttgtttaaatatatgactcaaactgccatgtatttctcacttttcactcaggactaaaccactttaaaggcaaatgcttccacagcagggactataaagaaccaggtgtattcaatggaaagcgtgtcctggtggttggcctggggaattcgggctgtgatattgccacagaactcagccgcacagcagaacaggtactactccccgggtactcgggtgactctcgttactgacagaagagttattatcgtttgaaag

SEQ ID NO: 186 - DNA, 1021 bp, Homo sapiens
  misc_feature (561)..(561): n can be c or t.
tcaaagaaaatccaacattaaaatgtatgccttacgataggcttgtgttcttatttgctgccttctctctctatgctgtgcagctaggctgtaattttaaatgcatgtcttggattttattctacaagaaaaggaatgcatctgtttccattccttacccttggctgggggataattttaatgttgggtttgaaccccacgaaagaatgttatatttgctctatcttttggtagaaattagattggtaacctcgtaggtccacaaaagtaaactttcactttaagggaaaatgagtaagcaagtaaatattgctaggactaccactgggaaaataatttaaaggctatgtcacactggaggttgggtaagtggtttagaggggtgcgggttaagacattcgggggcataatactaaggagagcatccccaaccctaaacatcttcaaaatgatcagggcttatgggcactatttgacgagcataagaacttaataatgtcaagagaaattttagacctatttaatacatttataagcaagttttgagccaggcttagactnttacctgttcctcttggtattcatcaaccactgcacaaaatcttgggcacgcctggagtccagatacttgctgtagtcactggtgaatgtgccctgtgaatggcgcttgtcctcgttcatctgatcaggatcactgagtgggtctgcctgggaagctgagaatgatctgtgaagaacagtgattggtacaacataaatctctcctcaagagtagactcacttgagaagcatcttcactacaaaatacaagaccatataaaacagtaaggcaggcatctagagtatttcaataggtagtttagaaagatcttccttagcttgtcatgagaatcccttcgttttagtatagttgcatacgctattattctgaattctagaaacatgtttctcaactgacttctttttttctgaaataggattaaacaaatctttttctactaattaatctactcatgattat

SEQ ID NO: 187 - DNA, 1021 bp, Homo sapiens
  misc_feature (561)..(561): n can be a or g.
tctgcctgtccgtctgcctgtctgtctgcctgtccatctgtccatctgcctatccatctgcctgcctgtctgtcggcctgcctgcctgcctgtctgtctgctgcctgtctgtccgtctgcctgtctgcctgtccgtctgcctgcctgtccgtctgcctgtccgtctgcctgcctgcctgtctgtctgcctgcctgtctgcctgcctgtccgtctgcctgtccgtctgcctgcctgtctgcctgcctgtctgcctgtctgcccgtctgcctgtctgtctgcctgtccgtctgcctgtctgtccgtctgtccatctgcctatccatctgcctgcctatctgtctgtccgtctgcctgcctgtctgtctgcctgtctgcctgtctgtctgcctgtctgtccatctgcctatccatctacctgcctgcctgtctgcctgtctgtctgcctgtctgtctgcctgcctgtctgtctgtctgtctggttgcttgtgcatgtgtcccccagccacaggtcccctccgctcaggtgatggacttcctgtttgagaagtggaagctctacngtgaccagtgtcaccacaacctgagcctgctgccccctcccacgggtgagccccccacccagagcctttcagcctgtgcctggcctcagcacttcctgagttctcttcatgggaaggttcctgggtgcttatgcagcctttgaggaccccgccaaggggccctgtcattcctcaggcccccaccaccgtgggcaggtgaggtaacgaggtaactgagccacagagctggggacttgcctcaggccgcagagccaggaaataacagaacggtggcattgccccagaaccggctgctgctgctgcccccaggcccagatgggtaataccacctacagccccgtggagttttcagtgggcagacagtgccagggcgtggaagctgggacccaggggcctgggagggctcgggtggagagtgtatatcatggcctggacacttggggtgcagggagaggatagg

SEQ ID NO: 188 - DNA, 1021 bp, Homo sapiens
  misc_feature (561)..(561): n can be c or t.
ctgctttccaaatcagcttggagagacaggctgactcctttccctcttcctcaggcatcctctctggccacgataacagggtgagctgcctgggagtcacagctgacgggatggctgtggccacaggttcctgggacagcttcctcaaaatctggaactgaggaggctggagaaagggaagtggaaggcagtgaacacactcagcagccccctgcccgaccccatctcattcaggtgttctcttctatattccgggtgccattcccactaagctttctcctttgagggcagtggggagcatgggactgtgcctttgggaggcagcatcagggacacaggggcaaagaactgccccatctcctcccatggccttccctccccacagtcctcacagcctctcccttaatgagcaaggacaacctgcccctccccagccctttgcaggcccagcagacttgagtctgaggccccaggccctaggattcctcccccagagccactacctttgtccaggcctgggtggtatagggcgtttggccctgtgactatggctctggcacnactagggtcctggccctcttcttattcatgctttctcctttttctacctttttttctctcctaagacacctgcaataaagtgtagcaccctggtacatctgtgatgtttgccttctactctcttctgttccaaaaagacccaggtcccatttaagggcagtaatgtgttacaggtgctgtgataaaggctgggtactggatagcttgtgggcttatgggaggaggcctgagatgggtcagggggagaaggtattcagcaggtggctgggggactgtgtgcagcagttcgctatggcctgcctgtggtgcccatgtgtttgtacgggagggttagcttgagaaggaatcagattataaaaggtcttgaatgtcaagccagagagtccagactttttcctaagggcaatgagaagccattgaggagttctgagcagagtagtaacatgatcagttatgcttc

SEQ ID NO: 189 - DNA, 1021 bp, Homo sapiens
  misc_feature (561)..(561): n can be t or c.
accattgttcctgttttgcaaagaaggcaacaggctcagagaaggccagtgcctcgccccaagacatgctagctctgactaggatgccatgaccacgctgtcccctgcccactacactcacccggtgtgtagccccaaggctcatagtaggaggggaagactccaaggtgacagccacggacaaactcctcatagtccacagggagcagggggcttgtggaggagaggaactccgggtggaaaatcacctggtagtgaaaaagaaggactcagcccaagtgccttatttagctaagccctgagatcccaaggtggcccagagagggtaaaaagcttgtctagcatcacacagcatgtgtttggcaggaccaatgttcaaacccaggtctgcctgcctcagaagccagggttctttctaaccacagcaatacctttgataaaacttataggggaatggagtgtgtgaggcccaggacccaaccccttccctctgccgtgcccaacccagccctgaccaaatgccctcaccttcaccctgtcggcactgctattgaagaggcngattcggcggatggtggtcaggatggggtctgaggagtcatccagcatattgtgggtgcacacaggggggaaagactgccgctgcaggagccacaagaagggtaaggggtcatggaagggacagagaactccctacttcctcatgagccatgcggaccctgggggagccaaggagaccacaaatgcaccggacgtggggcaacaaacccaagtgatcaccaggagttgtggattcccactagtacaacctgtaaaggttttctttcttttcttttaaattattattatttatttttgaggcggagtctcgctctgtcgcccaggctggaatgcagtggcacaatctcggctcactgcaagctccacctcccaggatcatgccattctcctgcctcagcctcccgagtagctggaactacaggcgcctaccaccacgcccggttaattttttgtattttta

SEQ ID NO: 190 - DNA, 1021 bp, Homo sapiens
  misc_feature (561)..(561): n can be c or t.
caaactcacagttggatggcacaacaattacatcctgtgtggtcagcagtgatggaggggccgcagagatttggaaacaggagggacacaggatacagatataggagggtagaaggcagacttcctggaggaggtgaaacctaacctgagtccctaaggtgataggaagcaggaaccagggaaggggagcctattctaacacagtagaagcagcaactgctgaggtctggatgaggggacctcaactgtggcccaaaaccccaagttcccattgtggctctgccaacaactggctgtgcgacccaggacaagtcctatctttgcactgtgtctgggtttccccgtgtgtaagatgaggcggttgctaggtgcttattggatgcattcctcaagtcccgccctccatctcctattcccctctcttctggtttagtgctttaggaaatgtggcagaaatctttttctgcctgtgtctaggaaatcataattcatgctggcgtaccctggttgttgaggtccctgaatccttgtgcccacactgctgaagactccttgtgtganacaagtcaggggacatctgggtcttgactccccagatgctccagctggaccctgctgccctcccttgcccaccctcttccattgtagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggaggtccgtgccaagcccaggaggggcggggttggagtggggactccccaggagacaggcctcacacagtgagctcacccctcagctccttggcttccccactgtgccgctttgggcaagttgcttaacctgtctgtgc

SEQ ID NO: 191 - DNA, 1021 bp, Homo sapiens
  misc_feature (561)..(561): n can be g or a.
tcatttttacacaggatgtacgcgttttgaagcacaaaactctccagtgatcacaggtcatagactgtctgatttttatgtgaaatcccattttaagagtaaaatataagtaacatagtaggctctagtctataaacaaagacttctatttatagtttgtttgccccctgagccccatctcatctgctggtggcatgcacatgctctttattaccagtgcgaatatagctgggaaactaatgccactcaccatacaggatggttaacatggacacgggcatgacaaggaaacccagcagcatatcagctatggcaagtgacatcaggaaatagttggtggcattctgcagctttttctctagggacactgccatgatgacgagtatgtttccagcaatagttagaataatcactacggctgtcagtaaagcagaccagtttttttcctggagatgaagtaaggagagacacgacggtgagaggcacccttcacaggaaaggttggttcgattttcagagtcgactgtccagttaaatgcatcagaagtgttagcttctccngagttaaagtcattactgtagagcctggtgtcatcatttaattgcattagggagttcgtagttgagctcaaagaagtattttcttcacaaagaatatccatgtctaagccagaacttgtagcagatgaggtgtagaaggactaacaggttatagtttctgctcaccattcaccttgatgtacccacactctgtaacactgaggctggtgtacatgctgttctcccggggctggatttttgtcttccattattacaatgatagttaaagaactgaactgtggtggctgtaagttttcttcattcacaattttaggagagtccactgtttggttttattattttctcaccaaaccgaggacaaaaaagcagaatgaacttttagcatagaggttgcagggttttttttgagcgctcgggaagataaatgtcctggacaaagaagaaaagttttataactact

SEQ ID NO: 192 - DNA, 1021 bp, Homo sapiens
  misc_feature (561)..(561): n can be c or t.
gaagattgtggaaaatgatggaagattccggaaagtggtggaagattccagaaaatgatggaagattccagaaagtgatgaaagattctggaaagcaatgaaacattccagaaagtgatgagacagtgatagagtctggttccaggcgaagtgggagaggatgggatttgagaagggaatgatccctcctcacacctctaggatgggaagcttagtggagtgaggggtgggtaggaggttacaccctgtgtcctctgtcgctctgtgcaggaggaggaggcagagaaagggaagggtcaggaaagccagcccatgtcccacccccactggactcaccacgtgatggcaggtgaagcccttcatgaccgaggcctcattgaggaactcaatccgctctcggagactggctgactcgttgaccgtcttcaccgccacgcgggtctctgcctcacccttgatgatgtccctggcattgccctcatacaccatgccgaaggagccctgccccagctctcgaaggagggtgatcttctctcgagacacctcccactcgtccggcangtacacagagcatggaaacactacttcttacttatctacacagcatccttggaggatcccttgggggtctgcagccaccttccacccaagccctcacccaaaccccctcgaaaacactcatgaaatgagttctgtgatccaggacccatgccgggcactgggcatatggccgagaacaggacaggcatctgcacccatggagagggcatggcagagactcaaggaaggagccacaactggtccaagatcctggccaatatgtcctgaggcaaacctgcatccccatccttcttgtctgatttcagacccttgctatggaatgatgctacttcccacctgagactactgtttctgcaaagtgccaaggggatggaagacaggttgtaataggttggggaaaaaaaaagccaggatacttggagctcttcccatgaaaaggtggagtctatctcaccacccc

SEQ ID NO: 193 - DNA, 1021 bp, Homo sapiens
  misc_feature (561)..(561): n can be t or c.
tgtatttttgtagagatggggtttcaccatgttggccaggctggtctcaaactcctggcctcaagtgatctgcctgcctcggcctcccaaagtgcttggattacaggtgtgagccactgtacccagcaatttataaggttttaagactcaaataactccttctaaagtgaaatgagtctcctgttgtggtgggaggcagacatcattcaacttagaggacacagctggaaagcaatgtgagaaactaagaaaagtaacaagctggtagattggcatttctgacccatcttcctgcgaagtcaggtatcaaggctttaagtactaatagcacagtacctgatgagagaagcactggaatcaaaatttcagcagaggaaggaggtaccaagtgcaactctgaaggggcatgctgaagtgtgcaggggcatgcccaagagtcaagggccttacctcatcaccatatcgccgataactcacttcatacagcacgatcagaccattgggctccttcggctcctgccacatcaagtggacgacgttgttctcaaagatttcatgcgncacagggccaacaatgtcatcagccttggctgtaaggagaggaagtgagaggcagggatgtaactcttggatgagatcccacttctgccacctgtccatggtgcaaccttgggctggtgacgtcattttcccacaacccattttcctcgtcagagaacggacatctaaaactcatcccacaagattgttaggaagattaaatgggttactttctgcgtataacttttttttttttttgagacagagtcttgctctgtcacccaggcgggagtgcagtggtgtattttctaaagtttacataatgattgcctatgactcataattttaaaatatgacctggcatggtggctcatgcctgtaatcccagcactttgggagctcaaggttggcggaccacttgagctcaggcattggagaccagcctgggcaacatggtggaaccatctctactgaaaataca

SEQ ID NO: 194 - DNA, 1021 bp, Homo sapiens
  misc_feature (561)..(561): n can be t or c.
gactgaggttcacccgggtgaaggcgctcatgcccccaggtccttgtgggccccccagcagggacgagtgggcagccagctctgctgccccttgaggcccagtcggggaagcagaggctgctgaggatgaggaggcagcagccatggtggccctgggcaggctcacctcctctgcagcaatgcctgttcgcatgtcagcatagcttacaggggcagctggcgaggtgtccacgtagctctgacggggacaactcatctgcatggtcatgtagtcaccccggctgctgggcactgcccgggtaggcctgcaaatgctagcagccccgggaggtgcagggcccagtctgcccatctcgaccccagtgctctcctgccaggctgccctccgcccggccccaggtccatcttcatgtactcctcagtgccagtctcttcctctctgggagctggctggagctgggatggacacctgacagaaggtgagctgtggaaagccaccgggccagacaagtagccagactgatcactcccaaattcaatattgacatattcccccgggcncttgggctctggagggtgcagcaagggctgctgctgctgctgctgctctcgggcccgaggtaaggtgctggccttgggatcccccagggacagcctcgtgggccgggccaggcggctattggtctgagcagctgtgtccacctttcgaggcagatggggctgcagaacctgatggtggggatgtggaaggctgggctccagcctagccccgcagtatcccccacccaggctgtcgctgctggtggaagaggaagaatcatctgctgttgcagcatagagaaggcgaccagagctagtggaaaggcggaggtgctgatgccgggcaccctcctccggctccccggggcgctgggtgtgcttaaaggatcttggcaatgagtagtaggagaggactggcttgtgctgggggtcctcagggccgtagtagcagtcggaggggctgctggtgttggagtccccc

SEQ ID NO: 195 - DNA, 1021 bp, Homo sapiens
  misc_feature (561)..(561): n can be g or a.
gattggggatctggtggaagcggatgaactcccgcacccgcagcatctgtgtgtggtagcgggctgtgcccgagtacagccgctggatgatggccgacacgttgccgaagatgctagcatacatgagggctgggggcgtgggcacgtggggccgtcagcctctgcagggaccccacccacccacagggaccctgctcaggccccgcaccaggtcagtgtctcagtctcagcgtcgacatgcccacgagacgcccttgtacatctgcgctccagcacaccccacccttcagtagtccccgccctggtgacccagcccccaaaccatgtcacgatggtggcccctggagtctctaagttccagggcctcactctggcccggctagcagcctcagtttcctccaacttgggttcctccaccgtgggctctccccgccgcccgcccctgggcacactcacagccaatgagcatgacgcagatggagaagatcttctctgagttggtgttgggagagacgttgccgaagcccacactggtgaggctgctgaaggtgaagtagagcnccgtcacatacttgtccttgatggaggggccgcccaggccgctgctgttgtagggtttgcctatctggtcgcccaggttgtgcagccagccgatgcgtgagtccatgtgtggctgctccatgttgccgatggcgtaccagatgcaggctagccagtgcgcgatgagcgcaaaggtgcacatgagcaagaacagcacggccgcgccgtactctgagtagcgatccagcttccgcgccacgcgcaccagccgcagcagccgcgcagtcttcagcagcccgatcagctgggggacagggaaggggcacattccgttgatggggcaaggggggcaagggaggaggggaggtgctgcggccctcagagcgagcatcagaggtcagatccccaaagacttcctagaccctcctcctaagaggtgaagcccacactgggcccagcacaggtgtctcattaatcttag

SEQ ID NO: 196 - DNA, 1021 bp, Homo sapiens
  misc_feature (561)..(561): n can be t or c.
catgctcttgcggaggtcacccacacgtagcatgaagcagaggcggccgtggcgcagggcgatcaccgcatgcttgctgaagatgagggtctcagccctgcggtgggcttgggcagtcttcatgaagatgcagccaagcatgatggcgttgatcatgagccccacgatgttctgcacgatgaggatcaggatggccagtgggcactcctcagtcaccatgcgccccccaaagccaatagtcacttggacctcaatggagaaaaggaaggcagacgagaaggagtggatgctggtgacacagggctcagcagtgccctcgctgggggccaggtcaccgtgggcgaaggcgatgagccaccaggccatggcgaagagcagccagctgcacaggaaggacatggtgaagatgagcaatgtgtgtggccacttgaggtccaccagcgtggtgaacacgtcctgcaggaagcggccctgctcccggatgttcttgtgggccacgttgcagttgcctttcttggacacaaagcgggccctccgctggcgggcacggtacctgggctnggcagggtcctctgccaggcgtgtcagcacgtattcctcggggatgatgcccttgcgggacagcatggctccggtgacccccagggaggggcttcccccatcggaggcacccctcggacgtggcctagggcctcactgcagagtcctctcggtgggcaccttctcaccctggggctgcactcagcctgtgctggcctcacttctgagataactccccaccagactcttccttacctccacctgggtcccacttcacttcttaataccagcctcaggccgggcgcggtggctcacgcctgtaatcccagtacgttgggaggctgaggagggcagatcactaggtcaggagttcgagaccagcctgaccaacatggtgaaaccccatctctactaaaaatacaaaagttagccgggcatggtggtgcgcacctgtaatcccagctactcaggaagctgaggc

SEQ ID NO: 197 - DNA, 1021 bp, Homo sapiens
  misc_feature (561)..(561): n can be c or t.
cacttcttggagccacagacgcaaagcagcagccctcggggattgttcttccccagccaccggcccagagtgtggctggtcaatcgtggggacccaggactggctggacgcacagctctagggcccagtacctcccacagcctctgcagccttgggcgggggagaggggtgagccagtcctgaattgggttgggaggagcagggacaaaaataacccagtacaggttcctgctgaggccagaaatagcatagtgacaagtgccttgtaacaccctggatgagcagcagggggaggctgagctgaggctggcccagcctcacaccaggccctggccgggctacataccacatggtccgtgtgtacacacgcgtgtggggggcccgagagaccatggctcaggacagggaatctggagagatgctgaacttgggcttggccttggccatgggcacgctgcgcttgcgcaggggcccgcgggctgaggcgagggtcagagcttccagtaggctgtggtcctcatcaagctggcgggccgtgcagagtggtgtgggcactttganggtgttgccaaacttggagtagtccacagagtaacgtccgtcctcctcagctacaatgggcacaaagcgctggccccacaggatctcatcggccaggtaggaggtgcgggcctgggtggtgatgcccgtggtttccaccacgccttccaggatgacgatgatctcgaggtcctggtggtggtgcaggtcgctgggtgccaggtcgtagagtgggctgttggcatcaatgacatggtagatgatcagcggggccaccaggaagatgctgttgccacccacgccgttctccatggggatgtccacctggtggaggggcaccacctcgccctcggggctggtggtcttgcgtaccacctgcatgtggatggtggcgctgatgatcatgctcttgcggaggtcacccacacgtagcatgaagcagaggcggccgtggcgcagggcgatcaccgcatgcttgctg

SEQ ID NO: 198 - DNA, 1021 bp, Homo sapiens
  misc_feature (561)..(561): n can be g or a.
atctggtattgtacaacacatgcaggtaagtaactgaaatctccaggagttggatgtgtagtattttgggaggagaccaggcttgggccacaaatgagggcactttgcactttcatcaaatccatgtctaccttgtcaatctgaataactgagagagggcaggtagatattttacaccttgaagatttgttttctggtcatgtaaaaattaaatataaacaaataaagaacaaagcaagagagacagaaaaagaaagagaatgagagacaaggaaagattgtgttggggggagaagagaagggtttgcccagctagggcactaaactttggattcattctccaggtttgccacatcaccatttctttctgtttgctcttcgaggttcttttcttcctcttcagtctccagttctgcatgttggttgagtttgctggatacagaccaactcaggggcagctctgccctgctggctaactcggccagctctttggcactaagggatggggtgctggtctcataggtctcatggaagctgttgtagtcaacttcgtagaacccntcctccagggtcaggacaggtgtgaaccggtaaccccacaggatctcactggtgatgtaggagcttcgagcttggcatgtcatccctgcagagagaagaatggaggctttagcatatgtaagtgtgggctttccatggccaaggagtcacagagagccaggaggagtactgcatgcagctgttgagactgacctgcatacgatgccacacttagtaggtgtcattcatgttgtagacacatgctaatgtgccatggagattccaggcctcttaagggagtcctggggaacaatgagagagtcctggcccacatcaagccacatttgcctgcatggccatgcacatgcaaaggaaatcaagtgtgcaaatgcacacaagttttcgcatgtgcatggctatgtctggtccactctgctctgggagaaccctgaagccatgactctggcctcctactgctctt

SEQ ID NO: 199 - DNA, 1021 bp, Homo sapiens
  misc_feature (561)..(561): n can be  t or g.
acgggtgccggtcaagagaggggggcaccccgtgcctccctaccacaccttctggaagacatagcccccgctggggccccagcccacgatggggtcggaggacggcttcccgttgatgttgggctgtgagttgatggtgaggatgccctggcggttcacccgcagcagctcctccttcagcaggctggtctcagccgccaggggctcatcgttccagggcaggcaagtcacctgggagagacggtgagctggctggggcgaccatcaggtttggcaccctgagtccctctcacggcccccaacaaagacccagcctgtctttgcctccctaagcccttccaggtggaggtctcccaacttacccttctccctttgccatgtccacagcatggaggggagggcacaggatggggaagtcacagccccgcagcctggcctgcagctggggtcaggccaggggcaggggatgaaccagggtccccactccagcatcactcactttgtgaccattccggtttggttctcccgagaggtaaagaacaaagacttcaaagacacttncttcactggtcagctcctccccccacatcttcagcagctcctccttgggggacttgctcttcaggtagaagaggtagtagtccttcagctccccaaaggcaggggaagaggaattgcccctggcagaggggtgcccagaggtcagggcacactcctgacagagggcagtgccaccacatgcccaggaggccattcctgtaaattctgcccctgactcctcccaggtcaaccacaagcatgcaaacttcttctgccctcccgctcccaagaacaaagatgtatttgcaaggaaggtctgcaggccctcaccagcggccgttagggaactcgtcccactcctgggtacggtagatgtaactctttggtctggaggcccagaagatgggacgtacatcttcctctcggcgcttggggtgggcgctgagagcccagggtaggggacgcctgggtgaggatgggg

SEQ ID NO: 200 - DNA, 1021 bp, Homo sapiens
  misc_feature (561)..(561): n can be t or c.
gccacctccctggattcttgggctccaaatctctttggagcaattctggcccagggagcaattctctttccccttccccaccgcagtcgtcaccccgaggtgatctctgctgtcagcgttgatcccctgaagctaggcagaccagaagtaacagagaagaaacttttcttcccagacaagagtttgggcaagaagggagaaaagtgacccagcaggaagaacttccaattcggttttgaatgctaaactggcggggcccccaccttgcactctcgccgcgcgcttcttggtccctgagacttcgaacgaagttgcgcgaagttttcaggtggagcagaggggcaggtcccgaccggacggcgcccggagcccgcaaggtggtgctagccactcctgggttctctctgcgggactgggacgagagcggattgggggtcgcgtgtggtagcaggaggaggagcgcggggggcagaggagggaggtgctgcgcgtgggtgctctgaatccccaagcccgtccgttgagccttctgtgcctgcagatgctaggtaacaagcgacnggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagaggtgggtgggaccgcgggaccgattccgggagcgccagtgcctgcacaccaggagatcctggggatgttagggaaagggattgtttcttttccttcgctctatcccagggcaggacagtatcaggcacttagtcagctctaggtaaatgtttgtacagggcacactctacacaaaatgggtaccttccattttgtgcaactacagtcacagagtcgtgatccccagattcaggttccccaggctggtaggctggcaatctcctctcactcacctcttatggtttgttgtggtt

SEQ ID NO: 201 - DNA, 1021 bp, Homo sapiens
  misc_feature (561)..(561): n can be a or g.
acccagaatcctgcagtttctcctgattaacagctaagtaaattctatagcactgtactgaaaatataaaaaatttagaatatagggctgatcatccctgatcctaagattgtcctctgaagttgattttcagggtaaatctttcatatccactttttaaattgccgattgtttcttatgaaacaagtagtaaaatgtacaaaagaaaaagaatctagcttaaattatagagttcagacatattttttagtaggaggaagaggaatagaataacaaaatagagtgtgaaatttggagtaaattgacagattttcagaataaaatgtttcttttttctctgtacatgttaaaaatatactttgtattgatactttcatgtgccatcactaatattacatatatagcatattaaagagtgacattttaaaccattgttaaattattcaacagggactaaataggaatagtttgccaactccacagctgaggagaagctcaggaacttcaggattgctacctgttgaacagtcttcaaggtgggatcgtaataatggcaaaagncctcaccaagaatttggcatttcaaggtaaaatctgcagagccttttaagaaacttgaatcaaatgcatctactttgtttctgtcaataatgtttcaaatagttctggaagcagaaaggaatggttgaagtattttaggtataggacaacatgtgtagtaataatatggtaaaatagagaaactgattattaaagagaagctaatgtgtcttgtcctaaaactttgataggctgggtacaaaatgtgctggatccctgagaacatgagatagtttagggaaatcaggatcaactcaggactggatgctggggaagtttttaaatcgatagaagtggccattacagggttagccaccaatccaatgaatagtatccaaaggtaggtctgcagaattactgacttctgaaaagaggagcacgtttccaaggctcatcacaattgttaggtttaaggtaacca

SEQ ID NO: 202 - DNA, 1021 bp, Homo sapiens
  misc_feature (561)..(561): n can be g or a.
ctcctttaacataagatatatgggtaagaaaattccaatttaatgatattcaaatatataaatatttgttgcatcctcaggtttctagttatgtgttaaaaaaatgatatgttgaaatctcttcaattttagaagaaccttgttataaagaacagagctaaaaatattagaaccacctgccctttagtgtaacaaaataaactagcctttttggtttacttaattacagtcttaccatcaaaaatatattctctaacttaaaaaaatacttttttggtaatatttgatgacatttctgatgagagcacataaaaataaaacaatacttaaagatgtggatataaaatgctcaaggaatcatcatttaaaaacagacggttcccttattgtttctgttcatgtcaaaaagcagggttttttttttacacagtctctgtagctcctaggaatttcatttctacagcagcttttggcctgtgggctgagccactcttcttttggaattctgcagcaatttcctcaaaagactttcctttggtttctggaactttaaaaaatgtnaacagggtaaaggccaggagcactccagcaaagaggaaaaacacataaggtccacagaagtcctggatagaaagcaaacacagactttgagttagcagttttttgaccctctcttctgttcagtaaatctgtggaatattaggctgcttaccgcaatgtactggaaacacagagctacaatgaaattgcaggtccaattgctgaatgcagctattgctaaagcagcaggacgtggtccttgactgaaaaactcagccaccatgaaccaggggatcgggcctggcccaatttcaaagaagctgacaaagaggaagatggctatcatgctcacataactcatccaagagaacttattctgaggaaaaaaacaaaaacaatagtgggactgagatcatttggctgctttttcctttagctaagtagcctctgagttcacaggcggcatacaactttttctaat

SEQ ID NO: 203 - DNA, 1021 bp, Homo sapiens
  misc_feature (561)..(561): n can be t or c.
atggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcngagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgagg

SEQ ID NO: 204 - DNA, 1021 bp, Homo sapiens
  misc_feature (561)..(561): n can be c or t.
gagtcccctccttactggggtccctgccccagcctgaggggagggaaagctctgcctaagaccgcctgcgtccagagtccagacctacctttccacaggcccctgactccttcctccctggcgatggttctgtaggcgtccatagtcccgctgtattttctgtcgctcctggatggcccgaggtgtatgctggcctgaaatcggaccttcaccacatctgtgggctgggcacaggtcaccgccatggctcctgtggtgcagccggccaaaatccgggtagtgaggctggagtctgggaggggcagagagagtgggccagtgtcccctactaagcagcattctgggacatgctgttctctgcggggctgcccctgcagcttccttgatgtccactcagagcctcctcataagcgtccggtaccagcttccccccgcccctggctctgcctctgagtctagacttccctggtctcttgacccacacactttcagccacccctttggtgttcagggacctggtcactcactgtccgcgcctttgggggtgtacacctgcttganggagtcatagaggccgatgcggatggaggcgaagctcatctggcgctgcaggccggccaccagcccattgtaggggctgcagggaccctcagtccgcaccatggtcaggatggtgcccagcacgccacggtactgcacgagccgggccgtctggaccgcctggttctccccctggatctgagggacaatagcagggggtgaggactcagatgggaaggcaagaaggggctgcgtgcacaggaaccctgctggggctgggcctgcctgggctgggcctgagaacaaccatgctggtcacagtagaaatcactggtgtctgcgcagcattttaccattcacaaagcagtattatacacatggcttggtgtttgatcctcagagtaaatcagagggacagattgtttttcccattttataagtgcttcgtggcttgcccaaggtcacacagttaattccttac

SEQ ID NO: 205 - DNA, 1021 bp, Homo sapiens
  misc_feature (561)..(561): n can be c or g.
aagaaaatcaaacttaactcggacccagagacattttagtatgtgttggaaactttagcatctggtcaccatcctccaaagaattatttggattggaactcggtcagagctgtcactcttcagctaggaatctaagaggatcatgtcttggatgttacggagtatagacaaccaagttccctgccctcaaaagcccgatcacttataagacagcttatggagctttgacagagggcagcagttgatggcattatcctttgaactcatagcttagttggactcctactggcttgtgggaccaaatctttccctaccacagttggctatagcaaaagttgtgaaaaatgccactaggatatactggtgagggaaaaggaggtccatttgtagttatagtataattgaaaagaaaagctctgaagaaaactctagcctactctttttcagcccaaggggaaggcagagcacctgctgacagatgctggcgtagcgagccagggcgttggcgttttcctggatagcgaggctggatggacactggtcggcaatcctcagcacagnacgccacttcccaaagtcaacaccatctttcttgtactgagcacagcgctctgagaggccatcaagccctgcaagtcacaaaagagagaaaggcttctttgtacctttgtacctgatccatggggcttctaataaagggaaggagttctccctttgcttagctttcaatccactgtgcttgaggattgaaaacagccaagcatatcagcattaatcacaacactgaaccagaagacttagatttaataaatagtgttttgacatacatactatctactccatatatagaatagaagaaaccaatagttaatatgatactcattttacaaaggtggaaactgaagctcctaatggttaagcaactttaccaagtttgaattgctcaagagtgacagagctgggattcaaattctgcttagctaacccaatgttgtgagttaatgcttgtctacttgggcag

SEQ ID NO: 206 - DNA, 1021 bp, Homo sapiens
  misc_feature (561)..(561): n can be c or t.
gccttagttttggtaatctgcaaaaccaagggccctgccctgggtgctgctctcccagtgcaaagtccctaactttggtgtgacccttaccagagtcaaggctgtctgggcctggctccttgtgacatccatcgccatctctccgaggggctaagaggtagatgctttgggaggcagagatgctcctgcctgctgaggcctagcacatgctgtagccttggagcgtaaggcccgcctgtggcagcaacggctgcttggatggagagctgcctgaggctggcagcccagggcttctgcactgaaagggctcagcctggcggctgctcaaatactctgccccctgccatggggtcagaggcagggcagaaagggagggtaggccatgtgggtaacagttgacagggccacggggacagagccatggggcagccggccacactctgtgaacatggggtagggattgctgcccagcaggagggggtgtgcagagccagcctacccatcttccattcctcagccttgtgcgggcagaaagtcaccaggctgccttggccacagaanacttactgaaatgcccttggacagggagggggtcctaagggggcctggcccgcgctggtgcaggtctggacttgctcttggaggcaaggggatccccagtggattttcatctgcagagaggttcgatttgcatttcatacaatccaggggtctgtatggaacttggggaaggggtggtggaggaaggtggccaactgatcaaaaacaaacaaaaaacaggggtatcattcttaattttgtgactgcaaagtccaggcctcaggcttgctttgggtgcctccatgggcatagaccatgacttccaggctctggcccaggcctctccttgggctcacctgggagtgacatccacatgctatgtacttgctggcacctgccaaagcctgctaaaattagctggagctggcaagtgggtcagggtatggagggtgccttgtcagaatgccaggtctctcgccaa

SEQ ID NO: 207 - DNA, 1021 bp, Homo sapiens
  misc_feature (561)..(561): n can be t or c.
ataaaagccccaggccaggccccggacactggtgtcctgggtcaccgttagctccaggaataagtaccctagaacccctcgagaggctggacactggatagccacagtgaggaggggtggtgggcagagggccagtggcaggcacagctgccctagccaggacccccaaggcccatgtgcctccttccaaggtgccccaagcctgctcgccttccctgcccccagccttagttttggtaatctgcaaaaccaagggccctgccctgggtgctgctctcccagtgcaaagtccctaactttggtgtgacccttaccagagtcaaggctgtctgggcctggctccttgtgacatccatcgccatctctccgaggggctaagaggtagatgctttgggaggcagagatgctcctgcctgctgaggcctagcacatgctgtagccttggagcgtaaggcccgcctgtggcagcaacggctgcttggatggagagctgcctgaggctggcagcccagggcttctgcactgaaagggctcagcctggcggctgctcaaatactctgncccctgccatggggtcagaggcagggcagaaagggagggtaggccatgtgggtaacagttgacagggccacggggacagagccatggggcagccggccacactctgtgaacatggggtagggattgctgcccagcaggagggggtgtgcagagccagcctacccatcttccattcctcagccttgtgcgggcagaaagtcaccaggctgccttggccacagaacacttactgaaatgcccttggacagggagggggtcctaagggggcctggcccgcgctggtgcaggtctggacttgctcttggaggcaaggggatccccagtggattttcatctgcagagaggttcgatttgcatttcatacaatccaggggtctgtatggaacttggggaaggggtggtggaggaaggtggccaactgatcaaaaacaaacaaaaaacaggggtatcattcttaatt

SEQ ID NO: 208 - DNA, 1021 bp, Homo sapiens
  misc_feature (561)..(561): n can be t or c.
caggcagggtctgcagtggtatcactgtgggcagagcctggggagggggccaattctgtgcacagggcaagggcgagaggaggggccagggatctagggctccggggaggggtcagcaggtcggggggagggatccacggggaggggttaccctgggtgaagaagtgagccttgtactttccagtccgcacagcaaaaaccccacggacctcgtctgggtaggacgggtagaagaagagagactgccgagggctctgggggcagagtcaggggtcacggggcggggcaggccccaagcactgcacatacctggggctgccagccctggtgggaggccctggacgtgcaccgcttcttgcccacccaggaacctgagaggtggcgccacttggatgccactcagtgcaggaggcactgaggcacagactctcaggcactgcccacactcaccccaggggaaggccaggacaggggccaaggatctgggatcaggggtcaccggccctaccttgcctgtgcccagcagcagggggctgaggtcaaagccatccaaggtgacantgggcagtggggccccagccagggctgccagggtaggcagcaggtccagggagctggccagctcgtgggtcacgcctgggggcaggaggctggtcagtcactcagttcgccatcaaggttggggtggtggggccagggttccaaggagagggcctgcggactgaccgggagcgatatgacctggccagaaggccaaggcaggctctcggacaccgccctcgtaggtcgttccctttccacaccgcaagagaccggagcagccgcctcgggacatacgcatggtctcaggtctgggacacaggaggcgctcatgagccatggagccacagcctctgagccaccgagggtgaccagtggccccacacctctaagtcacaaagcttgcccggaggtgcccagcatgagcccggcacctcccaggcctaccaagaccagctctctgtgcactgtgtctcctgac

SEQ ID NO: 209 - DNA, 1021 bp, Homo sapiens
  misc_feature (561)..(561): n can be a or g.
gtccagcaatgagtcacagacctatgcaccacctgcaaaggagccagagaaaacaaacgcccagcgcttttagcctgaaaatgagaatctggtttgctggggaagataaagggtgtcggaaaatggctgttgggtaaatcattgatgtctgccactaggaatgaaaggcaaatcaggaactggcacacatgctttcagggagatggctgcaagggagagggcaaagactgggaagttgcttatgtggtgccagactatttggaagatcatggattgcggtgtttgtgttgtgtggtcatcattttgttctttgtttacagaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcntcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcat

SEQ ID NO: 210 - DNA, 1021 bp, Homo sapiens
  misc_feature (561)..(561): n can be c or t.
cggggtcccagaaggtggtttaaggactggtgtggacacacacagttctgttgtctgcccagcggaggggcctcacggggggccgttgggagccagattgtcagcttttggatttaccagctgtgggtggcagtgggcgtgtaactcagcatcttgctgcctcagtttctctcatctgtaaagtggggataataacatttacctcataaagttcctgcgaggattcgatgacttgatacatcagttgcttagcacagggctcagcactcagtacatgttccctgtcaggaaggcagggaggcctcactggcagcatcaggacatgggacatcaggacatacaccgtggctctcagggaaaggaaaaagaccctctcccaggtgtacaagctcgattctaaacctcatgggaccctgcattgttcgctccctcattcattcactagtccatgcgtgtactcagtagtggcataagcagactgctcgggtcggacctgaattagcctcacccactctcctcctacactgtccctccccagggcacattcgcctcccaggtgangctggagggggacaagttgaaagtggagcgggagatcgatgggggcctggagaccctgcgcctgaagctgccagctgtggtgacagctgacctgaggctcaacgagccccgctacgccacgctgcccaacatcatggtgagcccctggccagcgggcactgagggcctgggggtggcaagcacattgccagcccagtgccccccggtggtcgcacgtggggagggaaggatccaaaggaggtctcgtgcacaggaagccgtcacctggagtttggctgatagagagagtttgctgggtcatctctgccaatactgagagttcatgggggctgctttggctagcagggagggcttgctggtatctaggccagtagaaagccttcgctgggcagcagaaggtgttccctttgtcattccagccagtggaacaagttcactgggtcatctaggttcattaggg

SEQ ID NO: 211 - DNA, 1021 bp, Homo sapiens
  misc_feature (561)..(561): n can be c or t.
tactaaaaatatataaattagctgggtgtggtggcatgtgcctgtaatcccaggtacttgggaggccaaggcaggagtattgcttgaacccaggaggcagaggttgcagtgagccgagatcgtgccactgcactccagcctgggcgacagagcgagactccatctcaaaataaataaataaatataataaaaataaataaacaaataagcttccttttgctcattgaccccagaatcccagagaaaccacacgtcccagcaaccctcgtggcagaataagccacagaaaacagcccaccctaagtgcctcgcctccagcaactgaagttgcacgagtcagcacgtgcccttctgtggacctcagaatagatcccttcatacaagggctgcaggagaaagcaggactcccagcaatctctggggtctgagctggcctggcaagctgcctctggggctgccaggaactgctatctctctgcacagaggtccaatccatacctgcgttgcaaagatggctctcttcatcatagtgaagtcttccttatccagcatcttgttcangtcgggaaggctcccactgcaaggcaagcagggggcatgcatgtgagaacggagtaatgagaggggttagtcagggcctaggagggcacagggctgagggtggggcactcacaccagtaaggattcataaagcttcctcccgaacttttccttcaccgtgttggccgtgtccctggaggaagcagagcaacagggtcacatacacaccagctgccatttactgttaggcttctttagttagtttgtttgtttattttgagacggagtttggctcttgttgcccaggctggaatgcaatggcgtgatctcggctcactgcaacctctgcctcccaggttcaagcaattctcctgcctcagcctcccgagtagctgggattacaggcatgagccaccgcgcccggctaattttctatttttagtagagacggggtttctccatgttggtcaggctggtctca

SEQ ID NO: 212 - DNA, 1021 bp, Homo sapiens
  misc_feature (561)..(561): n can be a or g.
ccctccccacagtactgtgcagccctggaatccctgatcaacgtgtcaggctgcagtgccatcgagaagacccagaggatgctgagcggattctgcccgcacaaggtctcagctggggtaaggcatcccccaccctctcacacccaccctgcaccccctcctgccaaccctgggctcgctgaagggaagctggctgaatatccatggtgtgtgtccacccaggggtggggccattgtggcagcagggacgtggccttcgggatttacaggatctgggctcaagggctcctaactcctacctgggcctcaatttccacatctgtacagtagaggtactaacagtacccacctcatggggacttccgtgaggactgaatgagacagtccctggaaagcccctggtttgtgcgagtcgtcccggcctctggcgttctactcacgtgctgacctctttgtcctgcagcagttttccagcttgcatgtccgagacaccaaaatcgaggtggcccagtttgtaaaggacctgctcttacatttaaagaaactttttcgcgagggacngttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggat

SEQ ID NO: 213 - DNA, 1021 bp, Homo sapiens
  misc_feature (561)..(561): n can be c or t.
gtacatacacacccatgtgatacatatacacatacccatagtatacaggtaacataaaattatacacacacaacacaaacacatattatgcacatacgcacataacacacacacacacacccacatacaggcattgtgaactagacacatcaccttacaatctgtggtttactggaaggacatggaacaaaacccccccagccacagcgtggaagtgccctctccaggcacaagattctgcctccatggggcgtggtagcagcattgcccacccacccagggctgagtgagcaggcctgccccacactgcgcccatgcacagccactccaggctgcctcccacactgcctgcaaggaccccagtggggactgcaaacgggaagtctgcatccagggccccagggagggcaggtggggctctggagtatagcactttctagaagggaagcaccctcttggttctgaacgtaagtgggtctgctcacagggaggggcgtgcagccaccccaggaccccagctgtccaaggagccagggaaaacgcacccacggggcacctacngctgggagcgcaaagaaggagatggcaaagacagagaagcaggaggcgatggtcttcccgacccacgtctggggcaccttgtccccatagccgatggtggtgactgtgacctgcagggagagggacagtggtcagccacggatgggactggagcctcgggagggccaactgcctaacccaaacccaccactctgatgagcggagaggccggcaagagaccctgaccaccaggacgaccccgtgtgactcggcgaaagcaccaggaacagagccgcgggatggcacatgtctcccaggctctcggcgtcacacacaaggtatgtcccaccagcacatgtaaggagcccagcacccacgaagggccaggcctgctggctgggaacgtgggcctgggagctcgccccacaccggctgcctcatctgcctgcctgtccccaggaggctgggcccctgggccacc

SEQ ID NO: 214 - DNA, 1021 bp, Homo sapiens
  misc_feature (561)..(561): n can be c or t.
agcctgggtgacaagagcaaaactccacatcaaaaaaaataataataaataaattaattaattaattaaataaaacaagagcttttctttttgcttaataagagagagtggtggtggtgcttttttattcctgaagatgggaagtcctcttttgcccactaacctcagaagaaagggatgaggtgtaccgtacaggggcagtcaccttctcctctgtttagcttccattttggcctcatgtctaccccaaagttgtagcttagatggggggaaaattcagaattttgcatagaccataggtagcaccccctagaaaaagaatgtttctccccagatgtctcccactagtaccctaaccatctgcttgtctgtctagtgaggacccttggagggctgctaaaatgatcaagggttacatgcagcaacacaacatcccccagagggaggtggtcgatgtcaccggcctgaaccagtcgcacctctcccagcatctcaacaagggcacccctatgaagacccagaagcgtgccgctctgtacacctggtacgtcagaaagcaangagagatcctccgacgtaagtgttttcatcctgcctctgcctcaacctgaagtgacctttgccctctcaccccattggctgcctcagtttccctttcatcgacaaggccttgtgagcacttggcagatatgaggaaggtggcaagtagatttggccttggtggttgctgtacaatggattggcttctgtcatgttcttcagtcacagcccccttgctacccagccagttgctctgaggagcctgtcagtgtatgcagcataccttaaactttttggcccctccttccacctccttctctttgaaaccaagtaggtgacagagtgaaatgtcttccctgagagaaaacccagcatctccccttgatacgtgaccatcagtcaatttccaaagaagacatttcgttgcagtcaataatattgattactattactgttaatttcctcctctctggaaaaagta

SEQ ID NO: 215 - DNA, 1021 bp, Homo sapiens
  misc_feature (561)..(561): n can be g or a.
ctgacttagctgggtgatattgggcgggtttcctctctctggccgtttcccacacctgcaggctgggagtggtgcctgctgcctcctgacagtgctgcagtgagcatcaagtgagacaagcccatgaaaaccctctgcagccccagaatgccacggaaatgcagcattattgtattgagctttgctttgagtttattatatcatcaaacatattattaaatgactgagttgggtggggggttggtcaagagggcctatacaagaccccaggattctgtgggacctgagattctagaattctgccaccctgattccaaagcaagagaagagtctctgacatgatcagggccagaaaactggctggagaggcagacagtacagtgcgttcatataaatgactctaattcaggtggtggcgtgagactgtgggcatgtgtgatgtgcaacagagcaggctggtgtccataagccaacgatggcacagtactcaccttctggggggcattgatgactccagtgttgtagccaaactgcagggagccaagcactgctcctcccacngccagcatgaggcgacccgtcagcttctgcggagaaacaaaccacactgttataggcgtgtctgggagcaggttactacagggcagggcctggactggcaagtttctgtgttcagatatcttgcctgactcttggcaccacaccagtctttctcccaggaaacttggccaattcctgaccttaggtgcccaaaccagcctagctgacttcaagatactgggctggccgggccatttcctggggagagaggggaagtatgatcttctctctctgtagccaggtctcagagagggagaggctttggattcttgggggtctcatttccctggtggagccatgcctagggtctggtggttctagactctctgactgggaggcccaggaaccagccctcctatgcgagggggcccaaattacttggtaggaatagcacagatatagataggagaagcaccctgga

SEQ ID NO: 216 - DNA, 1021 bp, Homo sapiens
  misc_feature (561)..(561): n can be c or a.
cataatttttctcaaactcggcggacggttcgtgtttgaaagagaagttgccattgatgctgagcggcgggctgaggggtccatcaaaggaagggctggtgcaatcagtcagagggctttcaaagaagggctccagcgctgcgctgtaggcgtgcggcggaggcttaacgtggaagacatgggagctgtccatggtaccgtaaggcggactgggcagcccaggcgactggtaggagtaggggtgtacagggaaggaagcgctggccgtcggcaggtgggggggcatgtcctggttctgctcaggcagaaaagtccgaggattgagttgcaggcagcccgcaaccaggttggtggtgggttgggataagcccttgcaaagcgtctgaacgaaggagaccaggtctgggcttttgcctgagcgcaggatctccgacagagcccagatgtagttcttggccaagcgcagagtctcgattttggacagcttctgcgtcttagaatagcaaggcaccaccttgcgcaggttgtctagcgccgcgttcagtccgtgcatgcggttcngctcccgggcgttagccttcatgcgtctcaatttaaaacgctccaggcgagccttagtcatcttcttctttttggggccgcgtctcttgggcttttgatcgtcatcctcctcttcctcttcttcctcctcttccaggtcctcatcttcgtcctcctcctctcccccgttcctcagtgagtcctcctctgcgttcatggtttcgaggtcgtcctccttcttgtctgcctcgtgctcctcgtcctgagaactgagacactcgtctgtccagcttggaggaccttggggctgaggctcgcccatcagcccactctcgctgtacgatttggtcatgtttcgatttcctacattcaacaagggagaggcaaacagaaagaaaagcagaaaaacgctatattcaaaagccagatacgccttcagcttccactccctaaacctgtacaaatgcttgcgaaaagtacc

SEQ ID NO: 217 - DNA, 1021 bp, Homo sapiens
  misc_feature (561)..(561): n can be c or t.
ggatttatctagtataacaaaccatcggtctgataatacatatctgatagtgttgctgtgaatataattgaggtaatacatgtaaaagagctggcacacaaaaagaagctcaaaaaattgttctttccttaccaggtgttgccctggttcctgccatatcgctccccaaaggtgctgtaggagccatcatagtgtttgtagttcaactgtctctggtaacctggaaaggaagattaacgaaacagcacaatggattaatgtgcatgctgagggtggagaaattactaaaagtaccttggcttctcttgtgacatttcttaaattttgttgtcatagattaggagtttctgagccttaaatattttattggaggttggagagtggatagtttccttgaaattaactatcatagcagctatcatagtgagctaagctaatgtatcataatattcataagtaactgaaacctactgggaaatccagttgaaataacattcaagttttcccttactcaagtaatcactcaccagtgttgagatagccaatggccttggacttganctctggagtaagctgctgtgtttcatttagataatccagtacatagatgttaggagcaaagaggaccatattctgctctccacagccatagggcatctggagaagattttgtgtgttttgcatggcagagcctaatatgtctcctagagaatgggagagatgggaagtcataaagcttggagattatcatctatcaaagtcattaagcagaaataattagttgagcttagaaattgagaatttttaggaaggatgattcttccagggatagaagtatgattgaaagcaataaacaagcccaaagaagaagagaagaaagaagttaaaattatagtattatttttagtaaatatttatgggaaataaaaatagtataatagaagctgttaatgcccggatccactaggggctggagactcacccaaaactgagacagaagctcgggcagattcttctacca

SEQ ID NO: 218 - DNA, 1021 bp, Homo sapiens
  misc_feature (561)..(561): n can be g or a.
gaaaacagtctgggttccctgtggaacatcatttctcaaaactgtattttggggcttgctttctcatttttcctttccatttcagatatccttactgctgtctttgggctctttaaacactgccttttttcctttttcgatcacacccaaaaacttttctcaaaaattacatgtaaatttaaaaatttacaaattaaatttaaaattgaaattttaaaaatcccgactctccctaatttcaggaagcatgcatttattatacataacaagacgtgaaagccgcaagagtttcagcctaaacactgaagaccccgcgaagtgaatccagctgctgctctacaagcagcaacaacaactgggaagccttctcagctacacttcggggcactggtccaaccccacgcaaaatccctcgtttcccttagcgtggtaagacggagcctgacctgagctccaactgtcctatctttttcaaatgtttcaaacttactgcctttgttcagcagaaccacgggcacggtgatgatggtgacaagcgcagcagcacccagcagtcccagnagaaccttccacggtgtctgcaagccgagcagatcaagtccaattagagggaagcgtgtggccccagtttccgtaggagggtcggggctgctccagaggcagcaggatttgcaggtgggagtgcgttagaagagggagaccgcgggctgggggtgggggtggcgtctggagtgcgccagttggagttctctaaggcgggtgcccttgaacttgtgccttcagagcacattagcgttggtttctctacccctgcccgggttcgggcgtgcgttctgtgagtggctctccgggacattcaaagctcgacgccagggtcctagcagaagccagggtccgaaagctaagcgagagctctgggacgtcccttcacctgtcagagggtggccttggggcttccgcctaaggggagtccctggtccggtttcgccagcttttgggccatttggggagtttggcgaag

SEQ ID NO: 219 - DNA, 1021 bp, Homo sapiens
  misc_feature (561)..(561): n can be t or c.
agaggcacaagaaattaccttgaaaaaatggaattcagtgactgccatctaggaaagacagtgatactgtccagcagcatgcagttccgagagctcaactcttaggccaccctccctccactctactctaggaacaaggagcattaggtctgttttctctccatacacctcaatcgctcgtcctctcgtcttattaaaacacagacacagaaccaaactttttgacagttaaagacaaacaattacatctaattaaaatgctaagagatcctgagctgttagagatgaggagagtagatagtatgacctgatcttcccccctcttttttttcctttaacagtattctgtttcagcataaagcacactttctgaagaggttcctggtggagactggaaatctgactgtgtcctgtggcaacacacagtcccttgcataactttggcttcagtccctggatctgtcctttgcagctacgtcaggttccatggaaggaggaaagagctggagggcagtatcactcagccaaagctcccatggggtcccatgctggcaggataangggttcctgctctaacacagctagcacctcttcagggacatgcttcctgtccaccaccacttcgtagacatactcagagaaccactcatctgtcatgcacaggtaacctggagaaaagaacagaagacttatgagtccagagggcaagggacaaagagcagaaaccctttttgtaggataaaccttttacaaaactaatattcatacatatttttcagctttcccatctgtaatttcatttaatctaaatcttattagcaattctgtgaagcagataggacaggcatggctctatttttagaaaaattagaaaaccgggtcttgagtaactaggtgatgtgcccaggtcacatggtgaggttcagagctgggccttggacctaaggctaacaccagatcctgtactgatgctctcttcctccgctgccttggtgatggtgagtgatgacctgtatactag

SEQ ID NO: 220 - DNA, 1021 bp, Homo sapiens
  misc_feature (561)..(561): n can be c or t.
catacgcacataacacacacacacacacccacatacaggcattgtgaactagacacatcaccttacaatctgtggtttactggaaggacatggaacaaaacccccccagccacagcgtggaagtgccctctccaggcacaagattctgcctccatggggcgtggtagcagcattgcccacccacccagggctgagtgagcaggcctgccccacactgcgcccatgcacagccactccaggctgcctcccacactgcctgcaaggaccccagtggggactgcaaacgggaagtctgcatccagggccccagggagggcaggtggggctctggagtatagcactttctagaagggaagcaccctcttggttctgaacgtaagtgggtctgctcacagggaggggcgtgcagccaccccaggaccccagctgtccaaggagccagggaaaacgcacccacggggcacctaccgctgggagcgcaaagaaggagatggcaaagacagagaagcaggaggcgatggtcttcccgacccacgtctggggcaccttgtccccatagcngatggtggtgactgtgacctgcagggagagggacagtggtcagccacggatgggactggagcctcgggagggccaactgcctaacccaaacccaccactctgatgagcggagaggccggcaagagaccctgaccaccaggacgaccccgtgtgactcggcgaaagcaccaggaacagagccgcgggatggcacatgtctcccaggctctcggcgtcacacacaaggtatgtcccaccagcacatgtaaggagcccagcacccacgaagggccaggcctgctggctgggaacgtgggcctgggagctcgccccacaccggctgcctcatctgcctgcctgtccccaggaggctgggcccctgggccaccgacgttgctgtgcgccggcccccaggagaccgggagctcccactgaggctggtcgtcaacaaagagcaggggctgggatgacgcgctgcttc

SEQ ID NO: 221 - DNA, 1021 bp, Homo sapiens
  misc_feature (561)..(561): n can be g or t.
tcagtttgtccagtaagatggggtggtctgtttccaccaggtccagctatccactggtggttctatggggagcagtgggggtggttaaaggagctctgtgtggccgggagcggtggctgatgcctgtaatcccagctctttgggatgccaaggcaggaggatcgcttgagcccaggagtttgagatcaggctgggcaatatagtgaaaccttgtctctacgacaaataaaattagctaggcatactggtggtgcacctgtggtaccagctataggggggcgctgagacaggaggattgcttgagctcaggaggttgaggctgcagtgagccctgattgtgtcactgcattctagcctgggtgacagagtgagaccctgtttaaaaaaaaaaatagaactctgtgtggctgaggacagctctccaggggcccccacactgccttccaaattcccctaggcggctacattgcactagaaactatatccacatcaacctgttcacgtctttcatgctgcgagctgcggccattctcagccgagaccgtctgctacctcgacctngcccctaccttggggaccaggcccttgcgctgtggaaccaggtgggcatcctccttccgttcctccaaatgggaatcttgcttctctggtgggaccaggaagttctcagtccatttcctatctcctacactctccacagtttatctgagttgggagggtccctctccaaatgtgtcttggggtgggggatcaagacacatttggagagggaacctcccaactcggcctctgccatcatttaactctcccagcctatcactcccatactggaattttccgttcctctccctcattatttcacccatcattgaactttttcaccaatgagagaatccacctgctggcggtgaggcatggcaggatacgagaaagtaagtgggggtggggatgtggcaggtgccagtttgttactaggagacagggtgggagagactagagtctgggagcagacgtggtaaga

SEQ ID NO: 222 - DNA, 1021 bp, Homo sapiens
  misc_feature (561)..(561): n can be c or t.
tgtttccaccaggtccagctatccactggtggttctatggggagcagtgggggtggttaaaggagctctgtgtggccgggagcggtggctgatgcctgtaatcccagctctttgggatgccaaggcaggaggatcgcttgagcccaggagtttgagatcaggctgggcaatatagtgaaaccttgtctctacgacaaataaaattagctaggcatactggtggtgcacctgtggtaccagctataggggggcgctgagacaggaggattgcttgagctcaggaggttgaggctgcagtgagccctgattgtgtcactgcattctagcctgggtgacagagtgagaccctgtttaaaaaaaaaaatagaactctgtgtggctgaggacagctctccaggggcccccacactgccttccaaattcccctaggcggctacattgcactagaaactatatccacatcaacctgttcacgtctttcatgctgcgagctgcggccattctcagccgagaccgtctgctacctcgacctggcccctaccttggggaccaggcccttgngctgtggaaccaggtgggcatcctccttccgttcctccaaatgggaatcttgcttctctggtgggaccaggaagttctcagtccatttcctatctcctacactctccacagtttatctgagttgggagggtccctctccaaatgtgtcttggggtgggggatcaagacacatttggagagggaacctcccaactcggcctctgccatcatttaactctcccagcctatcactcccatactggaattttccgttcctctccctcattatttcacccatcattgaactttttcaccaatgagagaatccacctgctggcggtgaggcatggcaggatacgagaaagtaagtgggggtggggatgtggcaggtgccagtttgttactaggagacagggtgggagagactagagtctgggagcagacgtggtaagaactaacttgttgaaagttggaccatacc

SEQ ID NO: 223 - DNA, 1021 bp, Homo sapiens
  misc_feature (561)..(561): n can  be g or t.
ccttttatttttcttccatggaattttccagttaacttgagaaagtggaatcgaattccgatgttgaattttccttctggccccattcatgtggcaggtggtgattcaggtactactgggggctgctcagacaaacctcctcatcagacatcaagaggctgttgcaccaggagggccggtaccgtgtctagaggtggtcggcatggggttggagttgtattacataaaccctactccaaacaaatgcatggggatgtggctggagttccccgttgtctaaccagtgccaaagggcaggacggtacctcaccccacgttcttaactatgggttggcaacatgttcctggatgtgtttgctggcacagtgacaggtgctagcaaccagggtgttgacacagtccaactccatcctcaccaggtcactggctggaacccctgggggccaccattgcgggaatcagcctttgaaacgatggccaacagcagctaataataaaccagtaatttgggatagacgagtagcaagagggcattggttggtgggtcaccctccttctcanaacacattataaaaaccttccgtttccacaggattgtctcccgggctggcagcagggccccagcggcaccatgtctgccctcggagtcaccgtggccctgctggtgtgggcggccttcctcctgctggtgtccatgtggaggcaggtgcacagcagctggaatctgcccccaggccctttcccgcttcccatcatcgggaacctcttccagttggaattgaagaatattcccaagtccttcacccgggtaagagaaatagtgttgattttagggagaataactcagcaattggatctggtatgtgtgtattcaactcatttgcagacaaattgtggttgttcaataccagcctgttgtgaattacctgaattgatagcatcctggagcgacactcaaaatgtgtcgcctgtggtgcagctggagcccggagcctgcgtgccaggccccggaggcccccgc

SEQ ID NO: 224 - DNA, 1021 bp, Homo sapiens
  misc_feature (561)..(561): n can be a or g.
aagcagtaccagcagccagaaaccgcataacaaatacattgggcaattgggagttggggatgttgactgaacctttgaacttgactgatccgaatgccaaattctaatttaaaaagggaaaactggatgtttgagacatagtgtgatttggtgaagcaaacaatggactcccaggttaaaaactctaattagtctcttctgctgagttcctgagttaaccgtttgtgtagtggtctcctagtatattttataacttacaaagctagaggatcaaagcaattatctagaaatacacacaaaactcatgtttggtataaatgtctgaacaattaaccaaactgtcgcaacagcttttttcattgatttgatctaacactgatatgcctcatagggtcatgagttgaaaaaacaactctaaagctattccacaagaagaaagataatatttttctaaacaagtgttaggaaaatgaaaatatgaaagtttctgtttatgcttatttatgaaatttgcctaccttccaagtgtgtccccaagccacccaccaaagaatgatgcantcattccaccaactgcaaagctggatacagacagggaccagagcatggtgattagttgagcagctgccacagtctcttcctcagcccaaggggttggttttgggttcattgagtatgagattgtgggcagttcatctgtactgttgataacatagttgttgatagcttttcggtcatccagtggaacacccaaaacatgtctatagtgagatattattacctaggagataaagaaaaatagctttactatttcaaacattctatgtatttttgtttttgtctttaaagtgtttgttacgtgtttaaatagtaccatctcaattatgtgttttatatacatataaacatggatagatttgtttacagttggccatatcctataaaagaaaggttataaattacattgccaacaagaaccaggcaggaacaaataaatgaagggaacatgtaatactttgat

SEQ ID NO: 225 - DNA, 1021 bp, Homo sapiens
  misc_feature (561)..(561): n can be g or t.
atgccctttggcctaaaccctggacttgactaagaaatgcagcctccaatgacattgcgggaaaagggaatctgggaacttctatgacacaattcagtcttgctgagcatttggggctaatatttaactctgaacatatattgacataggcaattcttccataacagattcatacaaaatttaaaaatgcatatagaagccttaatttttatttaaattcttttatttaattgtgttttagaggcagagaatagtgtgtctttttttgcctcttttataatttttatttttttttttcatttttgccactgtctttctttgcgctttctagggcattacatttttcttttccgttttctccatgtttcttagcgagattctctaaaaggttacttctatttccatcacatcatcatctagctccagcaggcctacttttcttcatttcctctattgtattttctgcttttcattcttgctgtctgctcctctctcatcatccttgcctctgtctgtttaatcctcctgtccttcattttccttttttgcctctgcattcancatttctacttccaatctccctcctctgctctttcttctttcctctgatctgcagacttgcttctgtcccctccttctgttcccctcctggatgtgtctttggccaacctttccttctctgagacttcgtgttcttgttggtagatgggggctgatactgtaaacatcacaaaaataattgcattgagaacaagtggttcccatggtgtccctttgaatgagctcagaatgcccaggctccatatgatgcaggagacagcactcatgctggagaggggtctagacctcagtcacaagacccaccattccagaactttgggactcatctcttgacacctaccccctccccagttagaaaccaagaggcgctgggtcacctgggaagagaaagaatgaatctgcctttgccccagcaagcacgctttcctgccacattcacctaaaagtcttttctgagatc

SEQ ID NO: 226 - DNA, 1021 bp, Homo sapiens
  misc_feature (561)..(561): n can be c or t.
ccatagcgaatgttttcagctatcgtggtggcaaacaatacaggttcctgactcaccacaccaatgatttcccgtagaaaccttacatttatggtcctaatatcctgtccatcaacactgacctggaataaaaagtaagtgtgactttcatacatttgtaattgaaagggcaacatcagaaagatgtgcaatgtgactgctgatcaccgcagggtctagctcgcatgggtcatctcaccatcccctctgtggggtcatagagcctctgcatcagctggactgttgtgctcttcccacagccactgtttccaaccagggccaccgtctgcccactctgcaccttcaggttcagacccttcaagatctaccaggacgagtgagaaaaaaacttcaaggcaattcacagacacaggatataggaactgactgttcactaggtttaaatatacatgcacttttttataatctctacaagaaaacatcagaaactcttcattcaatagattaattgttgattaatcatttatcactgtaccttaacttcttttcgagatgggtaantgaagtgaacatttctgaattccaaatttcccttaatattatctggtttgtgcccactcttcgaatagctgtcaatacttggcttctaaacagaatcaaattttaagagattactaggttacaataactacttttagtgatattttgtggagagctggataaagtgacaaagaaattgacttaactggacaatcttttagataggtggatagatggccaactcagacttacattatcaattatcttgaagatttcataagctgctcctcttgcatttgcaaatgcttcaatgcttggagatgcctgtccaacactaaaagccccaattaatacagaaaagaatacctgaggaatgtgaagaaaaaccatcaggctactgagatagtgacagcaattttttttcatacttcttctgtctttttctaacataggtaattaaaatttaaaatggcgaggcaac

SEQ ID NO: 227 - DNA, 1021 bp, Homo sapiens
  misc_feature (561)..(561): n can be g or a.
ggacaagagcagggctttaaatgccccataaatatgtgtggcaaggatgaaagcacataggactcaaagaggaacaaaggagcagaaaggcaggaagagttggtgctgccttcaaaggagagtaggaacgagggcaggtggtatcaggtggacctctatgtggtcctgggttacaaaggtgccaggaaaaagcaagaaatggaagagtctaaaaagcaatggaagattgtggaaaatgatggaagattccggaaagtggtggaagattccagaaaatgatggaagattccagaaagtgatgaaagattctggaaagcaatgaaacattccagaaagtgatgagacagtgatagagtctggttccaggcgaagtgggagaggatgggatttgagaagggaatgatccctcctcacacctctaggatgggaagcttagtggagtgaggggtgggtaggaggttacaccctgtgtcctctgtcgctctgtgcaggaggaggaggcagagaaagggaagggtcaggaaagccagcccatgtcccacccccactggactcaccacntgatggcaggtgaagcccttcatgaccgaggcctcattgaggaactcaatccgctctcggagactggctgactcgttgaccgtcttcaccgccacgcgggtctctgcctcacccttgatgatgtccctggcattgccctcatacaccatgccgaaggagccctgccccagctctcgaaggagggtgatcttctctcgagacacctcccactcgtccggcacgtacacagagcatggaaacactacttcttacttatctacacagcatccttggaggatcccttgggggtctgcagccaccttccacccaagccctcacccaaaccccctcgaaaacactcatgaaatgagttctgtgatccaggacccatgccgggcactgggcatatggccgagaacaggacaggcatctgcacccatggagagggcatggcagagactcaaggaaggagccacaactg

SEQ ID NO: 228 - DNA, 1021 bp, Homo sapiens
  misc_feature (561)..(561): n can be a or g.
tcccacctcctgggcagcctggtagaggagacattcctttaattcttcctgcctaatttagaggctgggtgggggtctgaaggttcactcccttcacatcatcccactagtctactttgggaagaattacaggttgttggagctggaagccccattctaggcatggtctgaagacctgaacaatcccagggggtggtgaagggggcagggaggagatgggcaccacttaccatttgaggccgcccagagaagtccttgcccttcttaataagcacctccttggccagctggtggtggccgacaatcactgtagtcttggtgcccatacgaaccgaatagatggggccatattttttctgcagcttgaagaagttgttatgcatatggccgtgtctggggaggaatggcaggctgcccaccaggggcagggacaggaggctcttggggtacttggcaccagggcaccttctcttgggccaaaacaaataagctagggtaagcagcaagagagccacgagctcccacatggtggctgggtgccggcaggcaagatagacagcngtggagtagaagagctgtggcaactctagggcacaaggaggccttttaaagggctaccctgatcttcaccttgactttgtgttatctcttgccttgtggaaagattctcctggagcccagccaggcctgagctcatatccagaagggagagaggcggtgggagtgaaggcctcctcaagggctggctcaactccagggcaaacctccggaggaggagctaggtaagggaggtcagttgatcaccctctgaggagctccccatgcttgaatgactccagagtgcgaatggtatctgggctcaggagtcaaggcttggaactttccatgttgcaaaatcaaaatcactggacagatgacagattcaggagggtcacaagtagcagggactgttaaaggtcttttatgcttctttttttttttttcagagtcttgctccatcaccaggctggtgtgcagtggt

SEQ ID NO: 229 - DNA, 1021 bp, Homo sapiens
  misc_feature (561)..(561): n can be c or a.
caatagctaggctaattctccccagcagctttcatggaggacagtagtcactgcccccattttccatgaaaagtaacatgaatcctggctgtataaggggcacttactgtgctgggtgctaggctaagtgctgtacatgcaccttctcagtccattagagaagtctaggctcagagagaggagtggagtgaggattccttgacccctcagaccactgtggtcctcccatcccacctcctgggcagcctggtagaggagacattcctttaattcttcctgcctaatttagaggctgggtgggggtctgaaggttcactcccttcacatcatcccactagtctactttgggaagaattacaggttgttggagctggaagccccattctaggcatggtctgaagacctgaacaatcccagggggtggtgaagggggcagggaggagatgggcaccacttaccatttgaggccgcccagagaagtccttgcccttcttaataagcacctccttggccagctggtggtggccgacaatcactgtagtcttggtgcccatacgaacngaatagatggggccatattttttctgcagcttgaagaagttgttatgcatatggccgtgtctggggaggaatggcaggctgcccaccaggggcagggacaggaggctcttggggtacttggcaccagggcaccttctcttgggccaaaacaaataagctagggtaagcagcaagagagccacgagctcccacatggtggctgggtgccggcaggcaagatagacagcggtggagtagaagagctgtggcaactctagggcacaaggaggccttttaaagggctaccctgatcttcaccttgactttgtgttatctcttgccttgtggaaagattctcctggagcccagccaggcctgagctcatatccagaagggagagaggcggtgggagtgaaggcctcctcaagggctggctcaactccagggcaaacctccggaggaggagctaggtaagggaggt

SEQ ID NO: 230 - DNA, 1021 bp, Homo sapiens
  misc_feature (561)..(561): n can be a or c.
gggtttcctgtttccttttctgatcattcttacaagttatactcttatttggaaggccctaaagaaggcttatgaaattcagaagaacaaaccaagaaatgatgatatttttaagataattatggcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtgacatgttcgaaacctgtccataaagtaattttgtgaaagaaggagcaagagaacattcctctgcagcacttcactaccaaatgagcnttagctacttttcagaattgaaggagaaaatgcattatgtggactgaaccgacttttctaaagctctgaacaaaagcttttctttccttttgcaacaagacaaagcaaagccacattttgcattagacagatgacggctgctcgaagaacaatgtcagaaactcgatgaatgtgttgatttgagaaattttactgacagaaatgcaatctccctagcctgcttttgtcctgttattttttatttccacataaaggtatttagaatatattaaatcgttagaggagcaacaggagatgagagttccagattgttctgtccagtttccaaagggcagtaaagttttcgtgccggttttcagctattagcaactgtgctacacttgcacctggtactgcacattttgtacaaagatatgctaagcagtagtcgtcaagttgcagatctttttgtgaaattcaa

SEQ ID NO: 231 - DNA, 1021 bp, Homo sapiens
  misc_feature (561)..(561): n can be c or t.
gggagagaggacctgtgacaggataaaggggctgccttatttaaacctggaaggaagaacgacagtataagcttccaggatattaatatcaggctaacatggacagttaagagcctttgccaggagatagtatgactgtagttcaatggtgactgagcacctgggatgtgctagacacaagagtgacttctaagggtcacaggagaagctgacgtcaaaaacttcacacaaggggaccctgagaggtcacagaagttcaagattctgaaagtagttctggattccaaggagcaggctggcttcaccacttctgacaggctctgggaagtaggagaaagtttgcctcaggttggagagagcagtaggggagagggtggtatccccaaagggtcagatttctactcttctggcacaaagaagaagcagagaggtaaagaataggtcagtatgagcaagggcaactgaccctttatgacgtagcaaaggagtggcagcaagttctgaatgtaacaaattctcctttcctttttgaaaatgtagaacacattaacaaatgcactngatcaaactgtggtcaatcagaaatcgctgcacaaaatgtcttcctattaaataaaaatcatacagtgctttgcatttgaatagtgttctatactttcccataattctctcattagccaccactgggaaataccctgttataattatacagataaatgtgcaaatgacagaagaatcaatttctaaaagaagaaatacaaacttttataatgggagagaggatatatttattatcactaataaaaaagcatatacttcacctaataaattaatactttgtcactaccaaagttataattactataacatttatatataatatacatttacattaatattataaatagtaataaattatgaatgttataattacaaattatgaattttaaaatgtaataaccataatatcaatactatattagtgatggtgttatacattgacacaatttttttggaaaac

SEQ ID NO: 232 - DNA, 1021 bp, Homo sapiens
  misc_feature (561)..(561): n can be c or t.
ttttctgtagactctccctccgtttgagcttatctgacatttgctcgccgtgagatccaggccttgcatttgtactggaccctgttcttacacaccctgatccagcccacttgtgtagtctgggagtctgggacaacctccgtccgcccttctagccgggtcactgcaggcaagccttggtgctcttgcctgcgacgtggaaatgatgcctgcctgcagcgctgtatagtgcagagcgggcgaggggcatagggaagtcactggcacgtggtatgtgttggcagggctgcttctcaccccaaaccaagggagggacaggcagggaggctgagagcagcggcttgccctggagctgtcaggtgggaggcagagggcgggagaggctgtgggctgcccaggtctgatccctgacccacttgccacccgtgccctcagttcttccccaatggagaggccatctgcacgggctcggatgacgcttcctgccgcttgtttgacctgcgggcagaccaggagctgatctgcttctcccacgagagcatcatctgcggcatcacgtcngtggccttctccctcagtggccgcctactattcgctggctacgacgacttcaactgcaatgtctgggactccatgaagtctgagcgtgtgggtaagggccagccctggctgctgcttcctcagctggaaggaccctccccagccctccctccccattctgtaccccccatcagctcccatttcggactctcttactgctgtcccttgtcactgggtgactccacccctggaatccagtaccccttggttcccaactaggactgttttccctcagtgttgctctaagcagcctctctccactgcccaatgccatgactgctccctgccctaggagatctgtggaccatgactgtccagtcagttctgggttcctggcatttcaggggcacccactgagaggcaagacagcctcagggaaacatggaatcaaggcagaatcaaggagatctggagtggcccg

SEQ ID NO: 233 - DNA, 1021 bp, Homo sapiens
  misc_feature (561)..(561): n can be g or a.
tgcctcaggtaagaaagacctgggcttccctggctaaacgcatgagtccctaggaggccaggaaagcccccaaaccccagcttcgggccctcctccctggcagtgcttcctgggccccggagcctacccactgaggactcagtgcaggagttagggtctggagagtataaatgatcagagtggctaaaaatttccaccacctcccagttctccaggcatttgagttgtgaactcacctgctttttctcccatcttggacccccctgggaaatgtcccccttgcccaaggactgggctaaaggcctgggctcatgggatttgggactctgcagaggagcagttcaggggctggaggctcaaacctccaagcaaggacccctgggctctcatgggccctgtcccccttcccagcaactaggctaaaggctgaaggtcatggggactcaactcagaaggggggctcgttaggagctgaggggggcccctctaggctctcctgggagcggggacggggcagggctccttactgcagaagggtctccaccacggctttctggtggnccgcctcctcagggctgaggttctccagctctttgaggatgggtggcgtgaagtcttccccatcgtcgtccgtctcgtcctcggagccccgagtctcccccagcccattgggcagctcagccagctcccctcgaccgccgccgcaggactcccccttgtccagggggccttctccagccaggaggtagggccccggctcacccagtgcctggatcagtgcctctttgctcagccctgactcgagcagggccgccaggagctccgtctgcagctggctcagtttagaaaccatggctcggctgccacagggccacgcggcccgggtccaccacgctagccgcctcccccaccgcgtgggttgcgtttgcctgccggccggcagacacaaaccaaactccttgcacccactgcccccccaaaaccccactagccaagccctgtgggcacccccaacccccaa

SEQ ID NO: 234 - DNA, 1021 bp, Homo sapiens
  misc_feature (561)..(561): n can be c or t.
cctcagcctcccaagtagctgggactacaggcacgtgcctccacgcctggctaatttttgtacttttagtagagacggggtttcaccgtgttggccaggctggtcttgaactcctgacctcaagtgatctgcccacctcggccttccaaagtgctgggattacaggcatgagccaccacgcctggccccagattacctttctaaaatctgaatagattttagaaattcatatggccctaagagtttcagagaaacacaggcatgcacacaaatgcatgcacaaccgatacacacccagacacgcactagggatctgctcacacaagcagtcgtgcacacacacagatacgtgcattcacatgggaacacactggcctgcagacaccctcaatcacggaaacacacttgtcccagagacacatgcagactgcaatgcctgccaggcacccctttcccctgcatccattgacagccaacctctatcatcatctcctgctgtgtggggcacagggcgctcaccgtgggggctctgcagctgagccatggtggccatgaaggggntctgggtcacatggctctgcacaggtggcatgagcggctgctggtaggaggggtgcagcggctgggagaactggacgggctgcagggtggtcaggctgctgcccatgctgttgatgaccggcacactctgtgcctgcgtggaggccaggcctggagtggaaggggagggaatcagctgggccccccagttatatcccacccctgcccaagacctcccaagggcaccacctctccttcccagagcccgtggtttggaggagggggcagggtggtcaggaaacagccctccactgggacctgccactaatttaagtggctctggcaagtcattccccctctctgagcctttagctctttgtctaggctagtgggagaggcaggcggtgacttgttcaaaagttgtcaaactgcggttccctggagccctgggttccacagcagtgcaaaggccatggggtca

SEQ ID NO: 235 - DNA, 1021 bp, Homo sapiens
  misc_feature (561)..(561): n can be c or t.
gtgtagatgcagtagcttttgcctgtgggatgggagggatgggagatgtgtccagaccctcctaggaggccacatgagtgtgactgttctcggcccaagtctttctcgttcctcagagaatttgcggggcccctgggcacacaagctgagatccacccagccctggtcccttggcaagaactgagggacaggacctggttctggggaaaatgcaggggaatgtttctcccttccacagcccccttgcgagttaggaggccggctcccaccccagaaggtggccaggttttcatgccttcctagagaaagctggggctcgtggcctccaccacaggaagacgcagaccctcagaaacaagtctgtgaagtcacaaccagccccagtttacagatgtgaaactgaagctccaaaaagtcaggaggtcactgagtggggaggtgatggagtgggaacagcccccagatctggctgaggccgaagccctggagagatccccgcaaggctcccttagatgcctgacattctgttcttcctgaagcctcactcccttctctcctggngcagacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcaggtgaggagcctcaatttcttcagctgggaaatgggcacacttgggctcatggccccaaggtctgtcttctccctgagtgggtaggtcccagagacagctgcccttcagggccttcaaggctcttctggttttgtaaaagactttgtgaatccaagaagagcatctattctaggaaccacatttactgatcatcaagctactggctgccgtttattgagctcttatcatatgccaggcacaatactaagtctttgtgtgtatttacccatccccttgagccc

SEQ ID NO: 236 - DNA, 1021 bp, Homo sapiens
  misc_feature (561)..(561): n can be g or a.
atacaccaaatttgtttactttgaatagctttctttggacagaggaattttgagtacttaatattttttgcatatttttcatactttccatcatgaacatgtatggcttttacatttaggaagaaataatgctattttttaaaggaggaaaaaagagaaaagagttggtgcgaataattgaagtaatctattatgcagtgtgtgagtaatgaattgatagataggatcatctgtagatttcaaggagctataatttcccctgtaacatgtttttcaacatttctctccccttttattataaaaaacacaaactctgatctacactccaacaaagtctgcttttatcacaaggatactttaaacatttgatcattgtgcagaatatttattctaaattactgagaccttattcactaatcatagttttcacaggctttattccaaccatattgatatgttagttcgagactacggatttaatacctggatttctcctctgtgtcttgaagggaacgttgccagctgccttgtaccagcattacaaataatccagccacaaantaaatgcttttcatttctgctgtctgtcagaacacagaatgggggtagggtgaggggggcaggcaaggatttttaaacatgtcaggctaaattaattagatttgactagataaatatcataagtagaaggaaaaagctagtgttatcacttttattctgattatattttcagcttaattttaaatagtgggttatattatttccccagattttttggaggcaaaaaaggacacaaaagatgtgttccaccattaagctttttcattaatgtagggacacttctgtttaataattagaaggctcatttccagactggaaattaaaatgtccacaatcaacatttaaaatacccactgtagatgatatgctacatatggttagcctgaatggcaccttatccatcatgccacccccctcactatcagtctggctttcaattaatagtccttcacttccaagc

SEQ ID NO: 237 - DNA, 1021 bp, Homo sapiens
  misc_feature (561)..(561): n can be g or a.
taggaattgtgcatcaggaaagtgaagaggattgctagacatttagtcctgttataagagcactaaagatttggcagtcaccaggtatggagtctcaggaggagcttaccgatggatggggcatagccattatatttgcccgagtccagggcatctttcattgcctgggtaacttcagggtctgtaggcaggtttccaaacacagtagggtcccctttttatgggaggaaaacacaaaaggagccaagaggttattctcccatgttcagtactcagactcacccccaactgccatcttctccaaccagcctgtgaacatgagagtagaggaggacaatgacagcccctcagtagtgtccccaactcaccaatggacagggaaatcatggttttgtttggatttggtttcaccttcatgttgtccacaatggctcggatggggttgaaagttttcttggccatgtctgagggcctcacagaccacctggcctttctgcctttcatttttcccggcacagagcttctcccaccaacgttgacatgcacgtccagaattgaggngaggttgcctttgctgctcatctgaatcatgtatgggtccatcactagcgaagcctgcgaggggaaagaagttccctgtgatgttgataacatagcgctgggggacagaggagctacatttggacctaaacattgggtgacttcactaaaagtgtctttccaaactctctctttatttttttttctactttctgttgtaaagtagctttactatgaatgggggagttttaagagtttttactgagatggaaaataaagcaagaacccattctacttaagtaggatttgctacacgcatctgcaattcctgtcaaagcttaaccatgctctatgtgaaaccaagaaggaataagatgaaaattgttcatcagtcaaagcataggttctccttcctttccatgcgagcctatccaagaaaatctacctaatgcttcttgtcatctgcagaggaccaggaaga

SEQ ID NO: 238 - DNA, 1021 bp, Homo sapiens
  misc_feature (540)..(540): n can be c or t.
ccaactagaatacagcttcctgagaggcaggatcttgactgacttgttcattcctaatttctcagcatctagaagaaggtatggcacatagtaggtgcttgttagatacttgctaataaatggaaataaacatatccctagttcctattccagctttttccctgctgttttgtcctccattcttccagcagacaacaggactagttccctgaccccctgcaggaagctaacaataccctagcctacttctaagcaaaacgtcgcagcttcaaagactttccatggagggcgatgggctgaggacaatcttgttcttcacgtaaaacacaggcccacaatctcaaatttataatttaaaaatatatatacttacaatgtctctaaaggcacttatttttcttaaaaatcatgtatttgtaagctgaactatcattttaacacaaaagctatcattcttgctcaatggagtcaggctgctcttggagtttctgtcctgggaggaaaaagggcagggtgtaggtacctgatggttttccacangtcgaagccatccagaggctttgtgccattggtgtgtcccctggccagcttcacgagtgttggcagccagtcagagatgtggatgagctcccggttcttcacgcccttctgcttcagcaaggggcttgccacaaagcccacccctcggacgcctccttcccacaggctccattttcttcctcgaaggggccagttattaccccctgccaaagtctgccctccgttatctgaaacacagtaaggtcttggcatgaggatgatgttaactcttaaatacatttaagaacagagactgtatgtacattgttactaaatggtgcttaaataataaaaaaaaagaaaattccttgccttttcccaccctaaattcccttttcccattgacatagcctttcattattcagacataagtaaggcccagtgtgatacatatctacctttaaatcctccatggagagagccactggaaaacaaggcagtc

SEQ ID NO: 239 - DNA, 1021 bp, Homo sapiens
  misc_feature (561)..(561): n can be g or a.
ctgtggggagcgtggctttgctactcaatggcaactggatttcaagagtttcaggaagggtgggggagcaagatatcaaaggctcaagctcactccccttcgtccagacagacttttcattttttgtttgatgaagattaggaagaaaagagtgaggattaggcctaatttactgcctctgtcaaaagccagcgcagagtagaagggaagggagtaagtggattatgaaaagaaaacaaacggagggaaagggggccgaggatgaactgcattcagtgatatttatttatctgattgcaaaaggaaaagaagggatctgttctaatggttcaccttcttatgaaccctggagctcccaaaaccctggcgaagtccttctgacactgctgtgaggtagatcggagccattccatggctaaagtgagagaggccactgcttgagagcagtaataagggaaccagagataaaaccccaaatcttggtcttttctaccctgctgctctcagcctgggccacagagcctggagaacactaaggtctcatcagggtttgggtggcanaaggaatggaaccaggggagctctctttgccctaagcactcactgactgcacaggcaagccgggtgatgggtgcccctaccaaagccagcctgctgctccacggcacctggacactaccactgagggaggagtgaagttcaaggctggggtttagaaaacatctctcagacagagagcaagaggatggtgaaaacccacttggtaaggatccctccttgggtcacatggcccagtcgtcaggttctggagggtagagtgtcacagccggggaatcccatgggactcattctgaacagaggccagaggttttccacaggttctgatcaacagagttgttgcttcttgtccttcaggcctaagaaactccccaagaagccctgggaaaaaaagtggagataatagaccctggggtgaaaggagcaacaggtgcactgaggggaatgacagagatcagagacc

SEQ ID NO: 240 - DNA, 1021 bp, Homo sapiens
  misc_feature (561)..(561): n can be a or g.
ctttagaaacggctctaggttgagaccgccggcatggatctccacctctactgcagacacacactggaaggcttcggaccagtcgggctgaggttcggagaagttgcagacgcagcggaaatcttcatcgtccagctcacaaggttctggcgtggtcgcagagacgtgcaccagcggcagcagcagcagcaacaagcaggacgcgcgctcctggggagagagcagaggtctaggaggccccatccaacccctgtggctcccgagtggcacgcgttcgaccccaagaccctacactcaccatggtcgataagtcttccgaacctctgagctccggacaggctctggaagtgctttacgttctttcctacacagcggcacccgccggcttccaggcttcacacttgtgaactcttcggctgcctctgacagtttatgtaatcctgggatgtcattcagttccctcctctgtgaaccctgatcacctccccacctctcttcctccgagccagcccccttcctttcctggaaatattgcaatgaaggatgtttcagggagggggnccgtaacaggaaggattctgcagggcatctagggttctgtgtctcctggcagtgtcctgatgactcaggcgccccaggcggtgaatgccctgttgactcgggagcctaagccttctctggtgggtgtgggaaaaggatgatcctcagtgccttaggccagtaccatactctgcactatccaaccccccaatccccctaccttatatcccagagaatctacttgattcatttctttgacttcttccttgtcttggtttatgttgatctcctgccaccaaatccaagtccctgaatatcctcagatatttaactgcatgttttgtggaagagattgtgaacctcatctgttggcaccaaggggggtagaattaggttcaagaaaaggaagttggtctaaagaaaaattcccccttcctttttttttccttgctcctttgattaagtaataactttctttctt

SEQ ID NO: 241 - DNA, 1021 bp, Homo sapiens
  misc_feature (561)..(561): n can be c or g.
gcagggctcagcctgcctccctgctgctgaggcccctaccaaattggaacccgagtagcaccagggaagcagggcctgcaggggatgccattctcacccctgcctgcaaaacgctgcagtgcccgagtctgctgtgggctggtgggggaagggcatcgctaggttggtggctgcccccaccccagcacactccccccattctctttagattgtctcacagggggacccacttggttctcattctgaactttcagtgaatggattctgctccctgccttgcgtgtgtacccttgggtggcctttgcccgtatcttagtctcagtttcctgagtttgggcaggaaggagaggaggggttctgactgatgagttacctcttctccctctccccacctcgcagggggctcctgagagtgtgatcgagcgctgtagctcagtccgcgtggggagccgcacagcacccctgacccccacctccagggagcagatcctggcaaagatccgggattggggctcaggctcagacacgctgcgctgcctggcactggccacccgggacgcncccccaaggaaggaggacatggagctggacgactgcagcaagtttgtgcagtacgaggtgggtgcaggagccgattctccctgcagtacgaggtgggtgcaggagccaagtctccctgcagcagctgagcaggtggtaggtcagggatgggctcaggccccgcttgaatctgccccctccctacagacggacctgaccttcgtgggctgcgtaggcatgctggacccgccgcgacctgaggtggctgcctgcatcacacgctgctaccaggcgggcatccgcgtggtcatgatcacgggggataacaaaggcactgccgtggccatctgccgcaggcttggcatctttggggacacggaagacgtggcgggcaaggcctacacgggccgcgagtttgatgacctcagccccgagcagcagcgccaggcctgccgcaccgcccgctgcttcgcccgcgtgg

SEQ ID NO: 242 - DNA, 1021 bp, Homo sapiens
  misc_feature (561)..(561): n can be c or g.
agcagagaagacaaataatagatactgcgaagataggatgattgaagaatgcagtgatataaatttgggggaagaggagggaggcagagcaaagaaattcaaggccttggccagacgtaatgtctcacaccttgtaatcccagcagtttgggaggctgaggcaggctgatagcttgtgtccaggagttcgagaccagcctgggcaatccagcaaaaccctgtgtctacaaaaaaatacaaaaattagccaggcatggtggcatgcgcctgtggtcccagctacttgggaggctgaggtgggagaatcgccgggacgtcgagattgcagtgagctgagatcgtgccactgcactcctgcctgggtgacagagcaagaacgtctcaaagaaaaaacaacaacaacaacaacaacaacaacaacaaaaacacaaggcctgtggttgggggaaggttgtaactctaaaaaagacccatgtggctacagcgagggacactgggtgtaggtagagataagaagagtgatactcagttctcacatcacggcggactgaatacaggccnggggagtgagagaccatccacccctgtgatctggggcaagtcaccagccctttcagagaagcttccgtcttctctgcaaaatgggacaataccttgcttcacaagcttgcaaggatcaaaagaactggtagtgggccgggcgcggtggttcacgcccgtaatcccagcactttgggaggtcgaggcaggtggatcacttacttgaggtcacgggttcgagaccagcctgggcaaaatggtgaaaccccgtgtctgctaaaaatacaaacattagcctggcgtggtggcaggtgccagtgatcccagctactcgggaggcagaggcaggaggatcgcttgaacccaaggggtggaggttgcagtgagctgagatcgcgcgctgcactccagtctgggcaacagatcaagactgtctcagaaaaaacaaacaaaaaagaactggtagaggaagcgctttgca

SEQ ID NO: 243 - DNA, 1021 bp, Homo sapiens
  misc_feature (561)..(561): n can be c or t.
aaaaaaaaagtggctggaactgccatcactatcctagagatggaaggttaggccaatgctacagcaaggtagctgtggtcagacactaagaatgctccttctatctggctgccagccaatggatctccattctggaccagcccacgagaagcaaacctcaaaggaaactaatctgaggtcttagctcaatctgtggggaacggcattaaagcctctccctctgagtgacctctgctagcttctctacctcctgcttcctcatctgcttctgctacacacccgcacactgaaaaccctgtatattgtatgagtcctccctgaaccccacatcagtcctgaggtgcaattctgcctagtcatctttcctcttccctcaacagcagcttactttatgttcttcaagcttcactgaggcctcttttgcaaatcctcccagatctcctcagctgggatggggcccctctaggcttcctgagccccatgcttcctcccttcatggcatctgtcataatgcagtgggattgccatgtaactcccttgactgtctccccaacacagagntgtacacttcacatctgggcagggtcaccatgactgtgtccaccattgccagcttggaacctggcatactggcatcagtaaatgtttgctgaaagaataaatgataacaagctgtcctgcccaccgtgacctttgggagaatgggcatatgcttttgattacctgcagggccatcaaggtgttggccagggcttgaccataggtgtcatggcagtggacagccagggcagccagaggcacttcctgcatgacagcagatagcatgtctttcatgatccctggggtgcccacaccaatggtgtcccccagggagatctcgtagcagcccattgagtagaacttcttggtgacctaaggaagcaagcaggcacttggaggatacagaatccaccagccaggggatccatgcactcagaagagggggcctttgcctgggcagaacacttctgggtatgacgca

SEQ ID NO: 244 - DNA, 1021 bp, Homo sapiens
  misc_feature (561)..(561): n can be a or g.
attggccttgttccccagggtggagctgtcacaaaatagagtgggaactgtctggctttcagcccaagagaatctgcatggcaagttgcattaacaaccaggcatttccggcagttcccaacatttctgggaattttctcatccaaacgactgaaagcccactccattctcttgcttcttactcatgctttctttgtataatggtaattatgttttaaaaaatcctgggctatgttgtttcatggaacaatttagaacttattggtcaaactctgaagcaaaggtatataaaaggtagttagagatgtttagggaatattcaaagcacatttttgggtcactcataattgatctttatattcatatatgtatatatatatatataacataatgtacccatcttaacatatcaaagctaaaccagtattaaaaacaactgactatggtctattgatacaatatatgatgcccaagtacactcttcattgctactgcatatctaaaatcatttatttatttatccatccatcaagagtgtattgagagcctgacaacataccngcatcaagccctggaggtctttttaaggctgagccaatatagctatggataacattctaaaactgatagcatattttcatgttttatagtctttccacagactagttcaaaatgaacactgcctgagaggggctttaagatgactgactagaggtactggacacctgtttccccagcaaagaagagccaaaatagcaagtagataatcatactttgaatagacatctaagagagaatgctggaattcagcagagaagtgacagaaaacacctgagatactgaaggagagggaggcaaggtagacagcctggctggaatcagctgggagcccagagagggtccctagtgagaggaaagggtaagtgagagattcccagtggtacatgttcccatgttgactgctgaaatcctagtcataagagtctctcaaaccccaaggaccctgaaactggtattcccg

SEQ ID NO: 245 - DNA, 1021 bp, Homo sapiens
  misc_feature (561)..(561): n can be t or c.
gtttaaatttgccgattagtttcgatgattcaccagtgcttgatgattaaggggtattggtgcagtgccactgagttgctgttcatagtctccagtaagggcagtacaagagaggagaaaagtaaagttgcacatcaggccaatacatttccatgtccctacagcccatgggtatttttctctgaagtttaaaattacagctcaagaagatcatatgtatttatgtaatctgcctttaaccaggccaccttgcttccctaatgctgttgtttttttcccttcgtttatttatctttaattgacacctgttgctattcttatgcctgctcaccttcacataaatgtcagcatccatgcaccatgtatgtcacacacacacacacacacacacacacacacacacacacccctctaaaagttctgatgagtatttgataaatagtagagttttgaggagagatggaggaaagtgtttacaagtttaactttttgaatttgcttttaactctctgctgttccctcacctgtaaaatctgcctcatctctgcccctctttcttcntgcaaacctcacttctcatagcctcctccagcagcactgacttctggagattccctgtcagtgaaataaaactggaaagctggtctcataataaaagcccaacagtttatgggcaaagcccaaccacctgtggttcttcaggtgtggttttcttgaggagtgcttatttaccctgccacattttcctctctttctctccaaggaggctttctctccagggtggattaagtgaaattatgctgttacttagggactgatttacatatttcttatccctcacactctgggtttctctatgttagctacatctaggaaaaaaatggggaaaaaaatcaccttgattggaagtgcagttaattcctgaaaataaagcctgatcacgagtggtaatcacagatcaattagttactggatccctagataatgcatccctgtcattgtgagacaaaagaggggaaag

SEQ ID NO: 246 - DNA, 1021 bp, Homo sapiens
  misc_feature (561)..(561): n can be g or a.
cccaaaatcttaggatgctgccttaaacatcatggtagaataatgtaactagctacccacgatttccttctttaattcattttgtgttttatctccccaggaaagtatttcaagcctaaacctttgggtgaaaagaactcttgaagtcatgattgcttcacagtttctctcagctctcactttgggtaagtcagtgccattagaccaagatttctcattctcgcactatagatatttcagactgaaatatccttgcttgtctggggctgtcctgcacaggatatctggcagcatccttgacctctacctgcaatgtgttcttccctgggcttggggtcatttactttacctcttggtgtctccctttccttaagtgtaaagtgtggatcataatgacctatttcccagatgcattgtgaggattcaatagcatggttcatggaaagtacctcatacagtgcttcttggtgcatactaagtgctcaataaagcttagttattctgattattattctactacaaaatgggtatactataatgttgtgagtgagtgtggataangtacctagtgggtggcagtcacaaaagagataaacaataagtcgctgtttcttcatacgtacttcttacttttgaaaagatgagaaaagtctgggccatgtcacaaacattgccaaaaataagacaataaaaagcacagttgtcagagttaaaccacaacagtaccaaactctaccatttcttttctttttctcccactagtgcttctcattaaagagagtggagcctggtcttacaacacctccacggaagctatgacttatgatgaggccagtgcttattgtcagcaaaggtacacacacctggttgcaattcaaaacaaagaagagattgagtacctaaactccatattgagctattcaccaagttattactggattggaatcagaaaagtcaacaatgtgtgggtctgggtaggaacccagaaacctctgacagaagaagccaagaactgggctcc

SEQ ID NO: 247 - DNA, 1021 bp, Homo sapiens
  misc_feature (561)..(561): n can be t or c.
gcaggactgcagacatgactcatggcagggtagctgctgaggcacgtcccatctcctttcagttcaggagaggctgtgggaagagggaagaactgagcacacatgaagatttggcagagggaggaggccaagtagggaggaagtggaataattgatattggagccagacatataatcagatgaaacctgggcaaaaccaaacgaggtccagacataaggagaaggagagcaggcgaaaaggcaatagagatctgtggcatgagataatcctatgtccgtgggattttcccatggatggtacaactggcacaggacgatgttattcctcccctctggtgaaaccaatatggcagcagaaggcagggagggtggggaggagggtgtagtttgtctgcacaagcatcatcagcatattttcaggagcttctgagagctgatgaaggatcatttgctgcagatactttatattcactcggtcagccaacttgtattgagcaattgctggggcacagcagtgagtgaggtgcgctacagaaacacagttgaaaagaatctgacttngccctcaatgaacctgcagtcaagttagaagcacagaggtcaacagacaaataagataaaggcattagtttctgtactggagcataacaccaatactgccattgctcagaatgtttctagaacccctaaaagttcagaactgtcttcagcatcatttcaggagccagacaagaaaaccagtctcatttctttattgtcatgacctgggtttgaccagaaacaatattactcacttggagcacctcactcctcagatctggctctagttctaaatatcaaaccattctcaaatagcaaagctttgtcacctccctatacatatctcatttaaatatgtaaaggatctgtaggcaattccaaaaagaaggctctaaaaatatttaaaaagcaatggtcgtaccttatagttttaccttatagtgtatatcaataatagccttgtaattaaaaaacaatcatc

SEQ ID NO: 248 - DNA, 1021 bp, Homo sapiens
  misc_feature (561)..(561): n can be a or g.
caggaagttgttggtgtttggatggatgaatggactaatggatggatgaataatagatagatggattgttgagagagacagagaagagaaaagccttgcccccaaaagctcacagactacttggagagagaagaaagctacctggagggagaaccagatgcatgaagcagtgcagatgtggtgcctaatgagtgtgtagtctggaagggcagcaaaagtcgagtggagtgagaggttcctgtgtcctggagcactgagtagagactccctcatgggggtgaatcttaaaggataaaggggcctctataatgaaaaggaggaggatgggatttctggtagaggaaattgcttgagcaaaacctccaaggttggaatgactatggtgtgttcagggatgttagcagacccagatgggtggagcgttgagtgtgtgtgtgtaggaaggaagaggggaggtggctggatgagcacagtgagacctgatttgattgagagccttgaacgccacgctgaataatggaggcaatgggacgccatagagggcttttgagtagacatatntcagtgtagaagggtgaatttcagatttttagacagaatagagtaaggagaggagctcttagaaatcatctagtccagggcttgtggcagagccctgaggttttaagaaggcatgtcaggggctaccatgacaggcacggagaggctgagtgaattggggttcttgccacaattcccttgcctgagattcaacaagagcagctgtattacaatctgtgcaaaatgtcattaggagaaactagttagtagctgggcgtggtggcatgcaactgttgtcccagctactcgggaggctgaggccggagaatcgcttgaagctgggaggcggaggttgcagtgagcagagactgtgccactgcactccagcctggatgacagagcaagactctgtttcaaaaaaaaaaaaaaaaaaaactagtcaggactctttcagatacaagtaatagaaaccaactcaaac

SEQ ID NO: 249 - DNA, 1021 bp, Homo sapiens
  misc_feature (561)..(561): n can be a or g.
taccaaagggcaagtagggaaacagaccaacagagatgttaccttctgaataattggacccaggaagaggagtgtaacctaagagaggaagatacttgattataccagtctttgtggatgaaaatatctagcagtattcatagcaaatgcagtaggaaggagagagttaatcacaaacagaaagtaagcagagagtgggaccaagagtggggatgggagttcagcgagtcactcactagagtggccagctctccgccagctgatcacaccaagagagaagatgatgaggcccaggcccagagtcactgcagacacagaaaccttcagggtctgcatgggggacagcccaggtgctgcaaaaaatagaaacttacttgacccagtttctgttgctcacccccagggcaattccatttattgcagccacctctcagtgggttaaaaggtcctttatcccagctccaagggtctagctcacaccacccactcccaagaaaatgatctttctcaaatcaaaccctcgtcccatggacctctactcctagagtaagcctggggaanccatctccccagaattagcatcctggcttccaggtcctctctaatacagtggggcctctcaaggcatcctctttccttcctttacctcaaagccacccttatcaggataaagggctcctcactgtcctctccattgcccccacggtaacaatgtttgcttccttactttctccaactgagcagcttcctattacactgtcttaccacatgtcttaacctccagtggatccatcctgtgagttatcctactacttgtgtaccttctacatctagatctcccatgtgtcctttcagagcttgtctccatcccactccacagcccctgcacttccttgggccggtcctgttctgaatcatgtcccactcagattcttttcccatgataaaatgaacactccatttctaaagggaggctcttgtgcacgctgtgaggagacgttccccaggaaagttcaagtga

SEQ ID NO: 250 - DNA, 1021 bp, Homo sapiens
  misc_feature (614)..(614): n can be c or t.
caaaaggtcaccccacagtccccactccaaggcaggttgatagcagggatctcagggtgcccatggatcaaggactaagtcagagtcggggtccctcaggccgagggtaacgtaggtggtgcctgccaggctctcctcgcccaggggggctgagaatgtctaaacccgggtggctgtgacccctaggcagagccagcccagcccttgccagggatggagaccggcctcgaggaggccaagccctgggggtccacaggcctgtgggcttcggggaggctctgctccctgtggccctgtgtggcccaggctgctgagtcatcagaacctcgggggcgccgcgggccccacattccgcccaggcctctctctgacccccttcccagcccatctgtgtttttggaaaacagagccagagccccccgcggccctgccagcttgcggctgctcacgctgggactcaaatcgcacccttctgtcttcaaagtccaccttcacttcaaagctcggtcccaccccagcccggcctccacagggccaccacctgcccacacccaggcccgctgctgcccagtttcggagggaccttgggcatcccctgatcctctctagagcgnggggttcctggcatgggcccgttacacatgggtggctcggtgggtggtgaggacggggctgggagaagatcctggggaccccatggtggaggcaatgaggcacccaaaccccaactccagcgatggctgcttccacggggccctccgagccctgaccttcaaggtgcaagaaaagctttcaggggcaggggtgagtggaaggtgggcttcctcccttgccacctggggggcgggcccaggacagatgctccgtgagagcacttcccaacctaggcccagctgtggggaaggagggagcaggcggctgggctccaggcagggggaagagttgcctgagaactcagggagagagggagggctggggcaccccatgccagctccagctgcagcaccagagctcagagcag

SEQ ID NO: 251 - DNA, 1021 bp, Homo sapiens
  misc_feature (638)..(638): n can be c or t.
attccctgaccagggccctgggacccaccgcacagctgagctggcccgagctgaagagttgttggagcagcagctggagctgtaccaggccctccttgaagggcaggagggagcctgggaggcccaagccctggtgctcaagatccagaagctgaaggaacagatgaggaggcaccaagagagccttggaggaggtgcctaagtttcccccagtgcccacagcaccctccggcactgaaaatacacgcaccacccaccaggagccttgggatcataaacaccccagcgtcttcccaggccagagaaagtggaagagaccacaaaccgcaggcaattggcaggcagtgggggagccagggctctgcagtcttagtcccattcccctttgatctcacagcaggcagggcacccaggccttataggaattcaccctggaccatgccctaaaataacctcaccccaaatacaataaagggacgaagcacttatagataccacagacacatgtgtttcatttttagttttgttaaaaaaaaattctgacaaatcagaaatgggggttcaggagtggtggtgatgcaaaagatggaagccatggggtgggggctgtcaggggtgggggcagtagtgtctccttnacccccaccctggtgtcctctcctgaaggacagacggtcacattccaaaatgggcgagtcttctaccgtgtctgttcaactgagaagaaaacgtagcatggtcagaataaggcatgaaaaggggaaagtgaggcaggaacacacggcacacatgcagacactggtgtactgcctgggttcagaggacggacgtgggggtgagggaagggatgtaatatgatgagagaagacagaaaccccacataaaggtcagaaaaacatcccaacacagcatcaaagaccagggggcatgaaccagtcaagtgtccattatgcatcagatgcccatgacctatgtgatgggatttaggacaaacacactaaggaacagggaggacctaaag

SEQ ID NO: 252 - DNA, 1021 bp, Homo sapiens
  misc_feature (573)..(573): n can be g or t.
ctctgaaggcttgcctggtgctcactcagcccgtgaagagggcctgctggtcctctggagcccacagccctttgtccagaggcgactcctaacctttagcaggctctgccctaacttacagtcccaccattgtctgccccacatcctgtctgcctgtctgtgctccattctggcccatcctaggtgtctctggctgcaaagcctttcctgggctcagccttctgccttgaacgggccctgaccatgagtccccatgtgcccagcccataccttttccctgtccagccaggagccaacacaggcctggagcattgcctgtggtatggcctgctcgctgctgttcccggcctgggtggtcacggacatgcagaggtggcactcagagtctcgcggcagccattctcctgtcggcgaccctggagatgtgagcattagggggaaagcaggcaaggccaccctacagaggtgtttggtttctgtcctccttggtgcattgcagtgggaccacagagggagagggtcatgcagtggcagggtagggggaggaggagagcaggcattgggctaaggagngggcagtgggctcacttgggccagcgctgtcatccatggagcaccggaggacgaggcggcagaccagctggggcagcatgcggcccagcagcgtgtcgagcaggatgacggagtagcgctcagccaggcactggcagatgccgcccgccaccagaggtaccacgcggcacacctgggccactgccacagctagcgcaccctggggcgggggcggagagaggccagcatgggaccttcacttggcaagcctccactctctgcccagcacccagctgggcacttcctacgcattccctcattctcttctagaagggagggcaaggctattcacaaataaggacactggggatcagagagtccaggggatgcaggggactcacacagggtcactgagtgtaggagccagcttcagacctacgtctggccccaaaggctctggcccacagct

SEQ ID NO: 253 - DNA, 1021 bp, Homo sapiens
  misc_feature (531)..(531): n can be t or c.
ggagagcagcagctggagggcaggctgggagcgcttgtgagggagaggagctatggacgtctgcttctctgccaagggagagagtgaggtaggcctgggcccgctgacttcagggtgaggccacagctactgcagcgctttttatttatttatttatttactgagatggagtcttgctctgtcacccaggctggagtgcagtggtgcaatctcggctcactgcaacctctgcctcctgggctgcagtgattctcctgcgttcaagtaattctcctgcctcggccttctgagtagttgggattacaggcatatgccaccacacttggctaattttttgtatttttagtagaaatggggtttcaccatgttggcgaggctggtctcgaactcctgacctcaaggatcctcctgcctcggcctcctaaggtgctgggattgcaggtgtgagccaccacgtctggccatactgcagcactttaaaggacggtgtctttttctttctcataaaagagaataggactttattagcantggtgcagacattgtattacacaggaatgggtccctagcttgcacaaccccagctgagctttcagcagataaatcacagcagaaatagaatcaccctaggactttcaatcaaaagctggaagtccaccttacagaaagacaaaaagaaacccctttttatatcttaacaaagcaatagctctcaagcagcagagcatctcgaggaagaaagcttgcccggtcgccatcccatcatgccagagcgtgcagtgtccacccttgactacgctggggaattgctgattttttgaaaaagcttaacttaacaatttctgatgtctatcttttagagttctgtatgttcccattttttattcttctgaattttgaattgcaagtagctgtaaaatccaatctttgagtgcatgggggtgggtgtgaggcggggctcagcttcaaccccctgtcctgtaaagcagtggctggtttttcctgagcccagccctgggag

SEQ ID NO: 254 - DNA, 1021 bp, Homo sapiens
  misc_feature (592)..(592): n can be c or t.
cagccatggttcgcggtgccctcggctgccctgggccagagctggggctagctttcaccttgttgagacccaggactctgtcccccaagcctgtcttcgccagcgccttgaccccacccctcatatactgtgtcctggaaaacgtggacacgggagaccacagccagggcgaggtatcgcccctccatccccccaggcccaatgagaagcagttggccaaggtgatccaggtggcagaggcagcatcagacccagtctcctgtcaggcaccaccttgggtgccggtccccagatgccctggcggggagtgtgcatgctcccggagcccccaggtcaccccatgtgagccaggcccacagagcttggctctgcaatgcctgctgggctgctgcccatgctccaccccttctgggaagctaaaagacagcccttcagtgtccagagacctgcctggccttggagcctgggtttcacatgcccaccgggctggcaggggcactcagctgcctccagccccggcggtcaccctggcattgggtccatctaactgctccccagtcacaaggcagctgctccccaagtctccccaaanctgctggcccctctagaagcctctgtccattcctggaggaccgagggcagcctgcatgccatcccgcacacagccttctgtctgggcatcctgccttcacacatgctgcacagggaggaaactcttataccacattccttaagcagagactgaagcctggagccaggcacatggcacatgctcccacccacccaggacacactgcggtgtggctgcctccaggctggccccctagattgcgtctgctcctggcatggataactggcgcctttgcctggccgttggggcagtgtttgccttcccctgtcggcagcaaatatttactgtcctccgtctccaggactctccaggcctgagcagaccccggggggatgagtgtggactcagcggtgctgagggtagccccctgcccttcgggtcctggtgccc

SEQ ID NO: 255 - DNA, 1021 bp, Homo sapiens
  misc_feature (601)..(601): n can be g or a.
ggcagaggcagcatcagacccagtctcctgtcaggcaccaccttgggtgccggtccccagatgccctggcggggagtgtgcatgctcccggagcccccaggtcaccccatgtgagccaggcccacagagcttggctctgcaatgcctgctgggctgctgcccatgctccaccccttctgggaagctaaaagacagcccttcagtgtccagagacctgcctggccttggagcctgggtttcacatgcccaccgggctggcaggggcactcagctgcctccagccccggcggtcaccctggcattgggtccatctaactgctccccagtcacaaggcagctgctccccaagtctccccaaacctgctggcccctctagaagcctctgtccattcctggaggaccgagggcagcctgcatgccatcccgcacacagccttctgtctgggcatcctgccttcacacatgctgcacagggaggaaactcttataccacattccttaagcagagactgaagcctggagccaggcacatggcacatgctcccacccacccaggacacactgcggtgtggctgcctccaggctggccccctagattgcntctgctcctggcatggataactggcgcctttgcctggccgttggggcagtgtttgccttcccctgtcggcagcaaatatttactgtcctccgtctccaggactctccaggcctgagcagaccccggggggatgagtgtggactcagcggtgctgagggtagccccctgcccttcgggtcctggtgcccagcaggggtccagcccagggaagagactgaggccaggacaggcagtgtttaagcctgagtttctgggaaaggtagccctgggcagaacttgggccgaacgttggccagtgtctctctccagccaggctgtgaggtagctgtttccaggatgggcacctttccacacccagcaatgtggccaggagccgccattcacgggtgcgaccagcagatggcatcagagcctcacttt

SEQ ID NO: 256 - DNA, 1021 bp, Homo sapiens
  misc_feature (629)..(629): n can be c or t.
agagactgaggccaggacaggcagtgtttaagcctgagtttctgggaaaggtagccctgggcagaacttgggccgaacgttggccagtgtctctctccagccaggctgtgaggtagctgtttccaggatgggcacctttccacacccagcaatgtggccaggagccgccattcacgggtgcgaccagcagatggcatcagagcctcacttttgatgcactccggccaccagccacgggtccaggttctggccaccacccagggtctgagcagctgcatcctgcccctgccgggcactcccgggggctgtggggcctgtgggggccctgccagacactcttgggggctgtggggggccctgccaggcactcccagggactatgggggctgtggggggccctgctgggcactctgaagggcatggggcttaggaatgagaggagctgtctgatgatgatggtgggggcactgcagaggcccccggcctgctcaggtccagtctcggcccctaagtcaagcctcaggccagcctctcaccagcctgggtttctcagagggccgggacaaatgttctgggtctctaatattccaagaaagcctctggctggactctgagccccacctgcgagnccctagaatcacagagagctagggtgagaagaccagggggactccgtcccaccctcgtcgtggctgagcccactgtggccggtggtggaccaggctgtggcctttgctgagggtccccagggcccctgggggctactgaggctggaggccagcggtggccaggagggtccctccctcagccactcaagccagaaggtcgagtcctggtttctatgtgaggagggggcttcaggggctgggacctgggggcaccgaaggcctggagctggggtccaggcggctgagggttagtgcgttcccacgctcccctccgccagcgccgtgaggagagggaggtccactctggaaagaatgtttgagggcaggggtagacagggtctgggaacgcggag

SEQ ID NO: 257 - DNA, 1021 bp, Homo sapiens
  misc_feature (563)..(563): n can be g or a.
atgcccctcctaacatgaaagggatttaagcaagccaattgcttatttctgcctgggccagggaccccagttcctgaccttctcaagagatatgaacctgacccttctgagtgtagaactgggctgtggggccaggagatgtgggtttcaatcccaggacccccactggtggctgtgccatcttgagcaaggcactttgtttctccgagtctctatttcttcactggtaaacaaaggcacaaatacctcttcaccacatcataaggggattaaatgatgtaggaaaaaggatgttgtatagtcgtgcacatagtagggcagcaggtccaggaggtggacggcccatccagggacccagcggagcagccacttccccacttctcaagggtggtcaccaggtatgtccgcagggctgccccctgcccatctccaaggcctgactggctgatctcagctacacattggatactaagtcctagggccagagccagcagagaggtttgccttaccttggaagtggacgtaggtgttgaaagccagggtgctgtccacactggctcccntcagggagcagccagtcttccatcctgtcacagcctgcatgaacctgtcaatcttctcagcagcaacatccagttctgtgaagtccagagagcgtgggaggaccacaggggtatagagagccaggccctgcacaaacggctgcttcaggtgcaggcctggggctgtgaacacgcccaccaccgtggacagcagcagctgggcctggctatcagccctgccctgggccactagcaggccctgtacagcctgcagggcagacaggaccttgtgcgcatccagccgggaggtgcagttcttgtccttccaaggaacacccaggattgcctgtagcctgtcagctgtgtggtccaaggctcccagatagagagaggccagggtgccaaagacagccgttggggagaggacggtggccccatggaccacgccccatagctcactgtgcatgccatatatacgg

SEQ ID NO: 258 - DNA, 1021 bp, Homo sapiens
  misc_feature (551)..(551): n can be a or g.
aggagaggaagggcgtggaaactggaatgatcctagtggggtgtcttggcatctcttggcctcattttccccatctgaaccatgaagctaaaactaggggatgtggattaaatggttcctacaactacttgcaaggagaccactctgtgtggttgcaaagaacactttgagaagctgtgtgggaaagtttccttcctagcagggtagactcagctaactgcaggtcatgtggccattgtggatgggttgggagctcaagtttggggcagaagggaattttttttggcagcagagtggcaagccctgccgccaggcaaactctgctcttcctcatcctcagaagcacttgctcactctgctaaatcaaagtgaaacgcatgtttacagaatattggtccaaaagggtctcagcatctcccactacccagggtggcagagcctcgggccggccttgctccccaagaagggctgactggggctctgtcccctgccccagggctcgaggtagtgtttacagccctcatgaacagcaaaggcgtgagcctcttcgncatcatcaaccctgagattatcactcgagatgtgagtacaaagcccccctcaccagcccctgttcctggggagagaggcccagacaggattcctggggtgactgggggctgttggggagacagacagaggggcctctaccagcttggctccctcctggtggcctgggagtcagcccagctcgcccctctctcctactgcccctcccttcagggcttcctgctgctgcagatggactttggcttccctgagcacctgctggtggatttcctccagagcttgagctagaagtctccaaggaggtcgggatggggcttgtagcagaaggcaagcaccaggctcacagctggaaccctggtgtctcctccagcgatggtggaagttgggttaggagtacggagatggagattggctcccaactcctccctatcctaaaggcccactggcattaaagtgctgtatccaagagctg

SEQ ID NO: 259 - DNA, 1021 bp, Homo sapiens
  misc_feature (548)..(548): n can be g or a.
ttggatagactgggggaaataagtcctgtgggacctcctgccttaaagaaagcaggcggagggccctaaaggaaatcaggcaaccagaccaaaagaatgtggaccaggtggtccatgctgtgtctcttgtgacccttcttctccctgccatgtcttttgggagagcccttgtgttgcaaaaatgagagtgtggtggtatggattggggtttaggcagaacagtactggccaagcagcgcctccctggacctcaattttccctctgtggaatgggctagcaatcctgggcctccccagggcgaaggaaagaccactcaggaagggcaccgtctggggcaggaaaacggagtgggttggatgtatttttttcacggatgggcatgaggatgaatgcttgtccaggccgtgcagcatctgccttgtgggtcacttctgtgctccagggaggactcaccatgggcatttgattggcagagcagctccgagtccgtccagagcttcctgcagtcaatgatcaccgctgtgggcatccctgaggtcatgtctcntaagtgtgggctggaggggaaactgggtgccgaggctgacagagcttcccatttcaccttgtgggcccttcccaggcagagcttcaggtgcccctcttcccagtcattgatacttagcggtcctggccccctttcctctccctgctggtggtattgcacgccaatgactcggccagatgcccagacccctgttcttggtttacctgcagaatattatctttgccaccccgcgggatggctcaacccactttcaggatgcaggtctcctaatagcaacctgatatagcagaaagacccctgggctgggagtctgagacctagttctagcccagccctgaacctcagtttccctttctgtgaaacaagaatgttgaacttgatgattcccaattttccttttgaccttgaaatggtagaatatttatccctttgaggtgactcggatggtagactctcagacaccatagcacacg

SEQ ID NO: 260 - DNA, 1021 bp, Homo sapiens
  misc_feature (544)..(544): n can be g or a.
ggggcagggctggtggtcagctggggcggggtgggagctggaggtccgtggtcaccagctgccctgactaatgtcgttacttgaatataaccctgtgaaggcaggaaccacgtctgtctggttcacttcccacggtggttgagacatagtgggcactccggaagtatttgttgaatgagtgaaagccccgctgggggaaactgggtacagctctttcctcagtttccccatctgcactctgggctgaatgctggggctcctcccaatctccctgaagctggacctgagcccagtagggacacacagggtccagccagcgtcctggcttcctccagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggtgagtgggtggggctgggctgctagggnatccagatggcatgtggtatgtgtgtgtgtgcacacgcatggggaggagggaggaaactcggaaacttggtggtgggcaaaagaactaagctggagcaatagcagtgaagtccagactgggcacagtggctcacacctgtaatcccaatcctttgggaggctgagatgtagcaggacgaaccgcagacaaaactcctcagacactgagttaaagaaggaaagagtttattcagccgggagcatgggtaagactcctgtctcaagagcggagctctccgagtgagcaattcctgtcccttttaagggctcacaactctaagggggtctgcatgagagggtcgtgatctattgagcaagtagcaggtacgtgactgggggctgcatgcaccggtaatcagaacgaaacagaacaggacagggatttttacaatgctctttcatgcaatgtctggaatctatagataacataactggtta

SEQ ID NO: 261 - DNA, 1021 bp, Homo sapiens
  misc_feature (542)..(542): n can be t or c.
gcaaatccatagagacagaaagcacatttatggttgccaggagctgggaaagggcaggatggggaatgactgtttattggatgtggggctctattttggggtgatgagaatgttctggaattaaattcatggctgcataacactgtgaacatactaaatgcccctgaattgtacactttaaaatggttaaagtggcaagttttcactaagcagtaaattaaattctactacaattttaaaaagactaaaaaataatttaaaaaagattaaatgagataacgcaaaaaagcattatctcgaaaatacagctgatattagtataattcttactaagttttaagagtctaaggtgcaggattctaagtttaaagggataggctcttttggttttttggtttagttatttggtttttttttttaatccattatccccacccttgggaggcccccagcacccagtctgcactagaggatggggcccacctcccttttctctccaggcccagccactgaccaccagtaccctggccaggggcaccctnggtcattgccctccgtggcccaaggaagggaacagaaacaacagccaagaagacaatagccgccgggaagtcctcacatttctggagaaatagagcccattaatgaatgaagttcctccagcctgatcggaggacggggtgctggggaggcctgggctaaagggctcacctccagcccccaccctggcagggccgatggtacatgctcactcagtgagggggctccagaggtctgtgggtacgaacccaagggctggtgcccaggggcaatcagcttatgtctctgagccttgggaaacagtgagggtcagcccggctccccacgtgcttctgggcagctttggtattggagcaggtgcaaactcgggactagggcaggaccccctgagaggcgactgagcaaggccatcccgactcatgtttccttggccctgcccggggcacagcatcctgcccacatccctgcagccctggctcct

SEQ ID NO: 262 - DNA, 1021 bp, Homo sapiens
  misc_feature (551)..(551): n can be a or g.
gggaactagtgccgccccagggccccaaggtgggcggttcggtgattcagagagggcagctctgtgttaggacacactggggccagccaggaagggtggaaaagatagggaccagcgtgagcatagaggctaagggaccatgggagctccaagcgcgctcacagtggggaccaggtcctgggggctggggacaccagggaggtgaaatacccctccagcgggtagggagggtgggcagaggagggccagcggccaggcatttgggaggggctcctgctctttgggagaggtggggggccgtgcctggggatccaagttcccctctctccacctgtgctcacctctcctccgtccccaaccctgcacaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcntgaaggacaaggtgtgcatgcctgacccgttgtcagacctggaaaaagggccggctgtgggcagggagggcatgcgcactttgtcctccccaccaggtgttcacaccacgttcactgaaaacccactatcaccaggcccctcagtgcttcccagcctggggctgaggaaagacccccccagcagctcagtgagggtctcacagctctgggtaaactgccaaggtggcaccaggaggggcagggacagagtggggccttgtcatcccagaaccctaaagaaaactgatgaatgcttgtatgggtgtgtaaagatggcctcctgtctgtgtgggcgtgggcactgacaggcgctgttgtataggtgtgtagggatggcctcctgtctgtgaggacgtgggcactgacaggcgctgttccaggtcacccttgtggttggagcgtcccaggacatcatcccccagctgaagaag

SEQ ID NO: 263 - DNA, 1021 bp, Homo sapiens
  misc_feature (554)..(554): n can be c or t.
agcttcctgagtagctgggattacaggcactcacctccacgcccagctaacttttgtatttttagtacagatggggtttcaccatgttggtcaggctggtctcgaactcctgacctcgtgatccgccctcctcgcctcccaaagtgctgtgattacaggagtgagccaccgctcctggccagaaatctcttctttattatgtctactgtccgttatccaactccagaaggtaagaacctccactgatacataaggacttgtataccccacgtgcctgcaacagtgcttggcacctagtaggcataccaaaatatataaatgttgaacaaatgaagaaagttaaagtaaaactagaggtccaaaaatatcacaaaagccatctatggtcgccttttccctacctgattttgctgagtggccttacttttcagtcctctacacagctggaacattaatgaacacagagggggaagaagtgtgtttactctaggatcacctctcaatgggtcacttggcaagggcatctttgcttcttcgtcagctccttttgacangggggtgaagggttttctgcaccacactttgaccacaagcatcaccaatttcactgaacccaacagaaatttggaccctctgggggctctctgcgtggcagggcccttttctttttctttgggcttaggctgcaatttgaaacaccactttcctgagccagcatcccccttgcagcgctgtcacagggaggcttaggcagccacgtggaagccacctaccccgacctttggcagaatttccaaacacaacacagtagctttaagttgattaatttggaactctgaccttggccccaaaaggtaagaatacataacaaggtattttattctcaaaatgtgtcaggataagaagcacttctgtaaatcgacctttttaaaatagatataattagatttgcagttgggggcagtaaagaaagggtctgaacagtggataacatgttgagaggttaattattaatgggcag

SEQ ID NO: 264 - DNA, 1021 bp, Homo sapiens
  misc_feature (548)..(548): n can be a or g.
gcagcctgttgtgccttgtgcctcgaagaggtttggtatctgccagtttctccctcgctgtttttatggctttcaaaagcagaagtaggaggctgagaaatttctctgttgaatacctgatttcacaatcaagttaaaggaaaggggaaaagagtattggtggaagcttcttaggggaggggactaataaactgagataattctctggttcatggaagggcaaggagtagcaaactatgacacattttgcaaatgtatcaccatgcaaatatgcattgttttcctgacaatcgttgtgcagttgatgtccacattaaaatactggattttcccacgttagaagaatgtttaaatttagtatatgtgggacaaagtggaagacacacagatttatacatgcacatacttttcttcattcacttctttgtacttaagtttaggaatcttcccacttacagatggataaatgggtacaatgaagggccaatagccctccctgtctgtattgagggtgtgggtctctaccttgggtgctgttctctgcctcnggagctctctgtcaattgcaggagcctctgaggagaaaattgacctttcttggctggggcagagaacatacggtatgcagggttcaggctcctgacggagttggggcaaccctggagataagctcacacaaccctgcaagaccaggtgctgttaccctagccaatctcatggatgaaccagatcaatgccagatgagctctgcctaaaatgattttttggtgaactctgaaaagtggaatattgtttctgtaagaatatccatctgagactctatctcttggtaataccaaccaagagttatcagtttctctttaaccgagacaccagcaaagtgcctgctccagggtactgcccaggggagccctccatttgtagaatgaatgagagtccaggttatgaacagtgcctggagtgtaggaacaccctcctttgcctctttgacaggtctgcatcataacactttttttttttt

SEQ ID NO: 265 - DNA, 1021 bp, Homo sapiens
  misc_feature (546)..(546): n can be a or g.
gaaataccatattgcatcaaacctaagacgccatcaagaataaaaggcacttttctttacattactacccagacgcaaacagagctgccaattcaaccatgatgagtcaccagttataggaggtttgatttcagagctataagagtgtatgtcctagaaccaatgagctatcgtagatccaagaatctacatatctgagttggaagggctgccagcccttggggcatgatcttccatcctcaaagacttcttcagatttgaagagcaaggggaaggactgcctggtgtcttaacgaagtgtctcctactcagccagtaggaccctgagcactctggggcatcctggcatctgttgcccagctaatggttcccaccagtcacccgtcccaacccatgccaccatccagtgcccagcagctctcagagatactcacttactacaggagacacactcgttttctcttagaaagaaacctgcatggcaggtgcacacggtgttctgtttctcctggcctgtagggagaagtgcggcacagctaaaggagnagcgcctgcacccccaccccacaggacagaggaagtgacgagggacagggtgggggcggccagagaggagttggttgtcagacccacagaatacaggagggggaaggaaaggaagtgccaccgcatggggaaggggccaacccctggggtggggagagggcttggcctcaggagagctgcgctcacaggagaggtgcacggtcccattgaggcagaggctgcaattgaagcactggaaaaggttttcactccaataatgccggtactggttcttcctgcagccacacacggtgtcccggtccactgtgcaagaagagatctccacctgacccatttctggtgaggggagaagatggggtatgagtcctgcatcctcctgtccctgcatccccttcctgacatacccctaagtgtgtgtctctgtaatacacactcacatccatgcagtgtcccaccaaaacacacaccttcctgc

SEQ ID NO: 266 - DNA, 1021 bp, Homo sapiens
  misc_feature (553)..(553): n can be c or a.
agatccagaagctgaaggaacagatgaggaggcaccaagagagccttggaggaggtgcctaagtttcccccagtgcccacagcaccctccggcactgaaaatacacgcaccacccaccaggagccttgggatcataaacaccccagcgtcttcccaggccagagaaagtggaagagaccacaaaccgcaggcaattggcaggcagtgggggagccagggctctgcagtcttagtcccattcccctttgatctcacagcaggcagggcacccaggccttataggaattcaccctggaccatgccctaaaataacctcaccccaaatacaataaagggacgaagcacttatagataccacagacacatgtgtttcatttttagttttgttaaaaaaaaattctgacaaatcagaaatgggggttcaggagtggtggtgatgcaaaagatggaagccatggggtgggggctgtcaggggtgggggcagtagtgtctccttcacccccaccctggtgtcctctcctgaaggacagacggtcacattccaaaatgggngagtcttctaccgtgtctgttcaactgagaagaaaacgtagcatggtcagaataaggcatgaaaaggggaaagtgaggcaggaacacacggcacacatgcagacactggtgtactgcctgggttcagaggacggacgtgggggtgagggaagggatgtaatatgatgagagaagacagaaaccccacataaaggtcagaaaaacatcccaacacagcatcaaagaccagggggcatgaaccagtcaagtgtccattatgcatcagatgcccatgacctatgtgatgggatttaggacaaacacactaaggaacagggaggacctaaagggtttcatgagatcagtactcactgtaggaggagatgtctatctcatcaggcagctcactaatattgacctcaaagcgatcctgcacatcattgaggatcttggcatcattctcatcggacacaaatgtgatagccaagc

SEQ ID NO: 267 - DNA, 1021 bp, Homo sapiens
  misc_feature (551)..(551): n can be c or t.
aggtgtgtgccaccatgcacggctaatttttgcatttttagtagagagagggtttcatcctgttggccacattggtcttaaactcctgacctcaaataatccacacgccttggcctcccaaactgctgagattacaggtgtaagccattgtgcacttggccagaatcctcaatattcacacaccactggagctgttttaaagtttccggctttctctgccacataccccaaaattattaaactgatatgattcaaagtcagtataaagtagtaagaaaagggtggtcttgtgttaagcatcatccatagcccaattacgaatcctcctgttacataggaactcaacactctgttacaccacagcaaactaaagcttctccaaaattaaagagactattggcctacaagtttcttatccctccaacttgccacaccctcactctcaggtctctttaccttggcttaccttgacattgggcatgtatttagagaagcgctcatattccttgctgatctgaaaagccaactcccgagtgtgacacatcaccagnacagacaccttaggcaggaagtatacggagacatatggtaaatgtagctcttcattatcccctctagggaagtgactgtcacaaaaacacacctgggccgataataaatgacttcaattctgtgatctaaatcatgaaccccacgcttgcgacagaacatcccccacagctgtcaggttgtcaagggtaacagaggtcatgtgctcatggctctgcaagcatcatgtagttaggacaaaaacacccttcccttatagtcctaaccaaaatcccctccccagcactctccccaaatatacctgcccagtaactggctccagctgttgcagtgtggccaagacaaacactgctgtctttcccatgcccgacttggcctggcacaggacatccattcccagaatggcctgagggatgcactcatgctggactaaaagttggggggggaggaagataaattagacttcagtctc

SEQ ID NO: 268 - DNA, 1021 bp, Homo sapiens
  misc_feature (569)..(569): n can be g or a.
gtgtaatgtattagagcaaatcctcttgattaggcttgagaatggagccatggagccccatttttttcccacccttcatgcagtagtgtttaattaaatatttaaaatatttaatgccctgcacaggcatcatttaattggaatgaacaactgctaactgctggcacagggctctagaaggccccagatatcagtaatttaccactgtttgcttgctcttgggataggaaggatccggggatcctagaggaggagctagggcagttgggtgctggaggaggcacatgggggctcagcacagccacttgtttgccagctggtggagcagtgtggaactcgccttcttgggaggaagaaacacgtctccagacttccataacaaagtacccagagttgctgggctagttacagttccaatgaccattcctccccagcaggataagcccagggccccaccctacctgggtcccccttctcgccccgagggccctctctcccatcccgtccatcgcgaccaggcaggccactctccactgagctacacatgaccagggtgcaagcactgggcnttgttctgtgggagtaggtcttcatttctgcttccaggtagcccaggggctgtgtgagcaggaccagtgcagagaggaggaagagcagcatggcctggagaggtgaacagaaagagaaaagacatgcttatgcttcatggacatggtttagggcttggctcagcttctagaggtgacaagaagcccccattccctccttctgtcctctgctatggggcctagagcagcaggaatccaaaagcagtttaaggacaaggagggcacaaggtctggatggagagcatgagttacccagctggaactctgacataggttgacagcagcatcccccattcccaggtgctcatgtcttcccttcttgtgccttcccttgggcactaagtttggcacagtggctaggatgtagcattcctcactggggccatctgtcacatcaagaagggttcattgag

SEQ ID NO: 269 - DNA, 1021 bp, Homo sapiens
  misc_feature (553)..(553): n can be c or t.
atggcacctgccctttggcaccccaaggtggagcccccagcgaccttccccttccagctgagcattgctgtgggggagagggggaagacgggaggaaagaagggagtggttccatcacgcctcctcactcctctcctcccgtcttctcctctcctgcccttgtctccctgtctcagcagctccaggggtggtgtgggcccctccagcctcctaggtggtgccaggccagagtccaagctcagggacagcagtccctcctgtgggggcccctgaactgggctcacatcccacacattttccaaaccactcccattgtgagcctttggtcctggtggtgtccctctggttgtgggaccaagagcttgtgcccatttttcatctgaggaaggaggcagcagaggccacgggctggtctgggtcccactcacctcccctctcacctctcttcttcctgggacgcctctgcctgccagctctcacttccctcccctgacccgcagggtggctgcgtccttccagggcctggcctgagggcaggggtggtttgctcccncttcagcctccgggggctggggtcagtgcggtgctaacacggctctctctgtgctgtgggacttccaggcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggtgagggtcctgggcccctttcccactctctagagacagagaaatagggcttcgggcgcccagcgtttcctgtggcctctgggacctcttggccagggacaaggacccgtgacttccttgcttgctgtgtggcccgggagcagctcagacgctggctccttctgtccctctgcccgtggacattagctcaagtcactgatcagtcacaggggtggcctgtcaggtcaggcgggcggctcaggcggaagagc

SEQ ID NO: 270 - DNA, 1021 bp, Homo sapiens
  misc_feature (601)..(601): n can be c or t.
gagctggcccgagctgaagagttgttggagcagcagctggagctgtaccaggccctccttgaagggcaggagggagcctgggaggcccaagccctggtgctcaagatccagaagctgaaggaacagatgaggaggcaccaagagagccttggaggaggtgcctaagtttcccccagtgcccacagcaccctccggcactgaaaatacacgcaccacccaccaggagccttgggatcataaacaccccagcgtcttcccaggccagagaaagtggaagagaccacaaaccgcaggcaattggcaggcagtgggggagccagggctctgcagtcttagtcccattcccctttgatctcacagcaggcagggcacccaggccttataggaattcaccctggaccatgccctaaaataacctcaccccaaatacaataaagggacgaagcacttatagataccacagacacatgtgtttcatttttagttttgttaaaaaaaaattctgacaaatcagaaatgggggttcaggagtggtggtgatgcaaaagatggaagccatggggtgggggctgtcaggggtgggggcagtagtgtctccttnacccccaccctggtgtcctctcctgaaggacagacggtcacattccaaaatgggcgagtcttctaccgtgtctgttcaactgagaagaaaacgtagcatggtcagaataaggcatgaaaaggggaaagtgaggcaggaacacacggcacacatgcagacactggtgtactgcctgggttcagaggacggacgtgggggtgagggaagggatgtaatatgatgagagaagacagaaaccccacataaaggtcagaaaaacatcccaacacagcatcaaagaccagggggcatgaaccagtcaagtgtccattatgcatcagatgcccatgacctatgtgatgggatttaggacaaacacactaaggaacagggaggacctaaagggtttcatgagatcagtactcactgtaggaggagatg

SEQ ID NO: 271 - DNA, 1021 bp, Homo sapiens
  misc_feature (564)..(564): n can be c or a.
tttcaatcaaaagctggaagtccaccttacagaaagacaaaaagaaacccctttttatatcttaacaaagcaatagctctcaagcagcagagcatctcgaggaagaaagcttgcccggtcgccatcccatcatgccagagcgtgcagtgtccacccttgactacgctggggaattgctgattttttgaaaaagcttaacttaacaatttctgatgtctatcttttagagttctgtatgttcccattttttattcttctgaattttgaattgcaagtagctgtaaaatccaatctttgagtgcatgggggtgggtgtgaggcggggctcagcttcaaccccctgtcctgtaaagcagtggctggtttttcctgagcccagccctgggaggtcgtggtaggtgtggaggctgcagagctcctccagatgctgccctcgctgtgcctcacaccagagaggatggaagtgggctctggtgtcagactgtggttgagctgagacagacaaggccgacacagggctgggggcccgtggtccaccagtggaagtgactgccgaggaagggnggtgaggagggcggtgtgggagctgaggcttcttttcagcctggcagctggcgagggccagggagcaggggaagagcctggtcaccatggtcccagagcccgtctcacttggcttttcctttgcagctgaggaggatgagggccagagagggactgtgtgtatgtcctgcctggggacccacagccaggtgatagcagaggtggtttgaagcccaggcctcccacgccaacccactggtcttgctgtttcagcagggaaggccgggagccctaggagctggggaaaggcgactgcccgggtcctgggtgactccccacccccagatccccagctgtcatcactggggcaaggacacattaaactggtccctgtgggtcaggtctgagtgggggaggacctcccctccccactgcctcccacaggggcttgtgatgcagggtttcaggaacagggctg

SEQ ID NO: 272 - DNA, 1021 bp, Homo sapiens
  misc_feature (607)..(607): n can be c or t.
ttggtttttgttgtattcaattctaattatttattacacagttaccatcctttgatgagatgttactcttcatctgtgattgcttatagttgttcgcgagcttctgtccattggtaattagaaagtttatttatatcaagtttaatcttcctgttaaaaacagtgttctaatagtcatccatattaaaatattatatggcagtattaaaaactacaaatattactcttgggaatcaaatcatacactgtagcacatcatctttcttggcaatagtactgctgttgtacactgatggcctctaacagagaagaaatcattccattgaaagaaaagtaactatcaagaacaaagttggaagtgatgccttaaagctaccggcccatgtctaaatgtacttttgatttttattttattggttaagtagaaattatttttaatgtaatgacagcccattaataaatgtctcctctgttgaaggtagggttaattcagtatgccaataatccaagagttgtgtttaacttgaacacatataaaaccaaagaagaaatgattgtagcaacatcccagacatcccaatatggtggggacctcacaaacacattnggagcaattcaatatgcaaggtaagttttggtgctaataggccaatgttttcataatgtaaaacattatatttatgtaataaatatgaaaaagtaaggaaaagacaaagaaaaataatatacctggtacctaatttaaatcagaactaataaagaaaaaaacatcagagcattctatgtcttgaatactttgagaaggcagctgggaaagttaaatctttgattttaggatatttataagatatcacatgatatttaaatgaatttatgtgaagtaaatgaaatgagaagaccttagattaaaacagtaggaaatggggcaatctgtcataatttgttaatattcatcagagattcagacaaattgagctcatggatcacttggtgcaaattaacaaagaccacagaatcttaa

SEQ ID NO: 273 - DNA, 1021 bp, Homo sapiens
  misc_feature (561)..(561): n can be c or t.
tggatctgcagctccagagaagggcctgggtcagatgtcactgaagccctatggtggcggaaaggcgagaaatagtgggttgagattccaagtgcaatccactgcggctcctcgctcgccctccaggtggcagcacaaccctgcgcttccgaagcccgttttctgagccagacactctccacgctctgggtatttcggcttctctctccccacacgccgaccctaggtcgcgcactttctgcctggcagaatttggccgaggatccaaacccggagcagcctccagagagcgtgtcgttcacgcggccagcatatgctcagagacctcagaggctcagagacctcagggctggtggtgtggtcggttgtgaccacttgtccctcggaccggctccaggaaccaacctggggaatgtgtgtaggggaagggcgggatagacagtgcccggagcagggaggcgctgaaagacaggaccaagcagcccggccaccagacccgttgtgggaacggaatttcctggcccccagggccacactcgcgtgggaagcatgtcgcggacnctttaaggcgtcatctccctgtctctccgcccccgcctgggacaggccgggacgcccgggacctgacatttggaggctcccaacgtgggagctaaaaatagcagccccgggttactttggggcattgctcctctcccaacccgcgcgccggctcgcgagccgtctcaggccgctggagtttccccggggcaagtacacctggcccgtcctctcctctcagaccccactgtccagacccgcagagtttaagatgcttctgcagcccgggatcctagctggtgggcggagtcctaacacgtgggtgggcggggccttttgttccagggactcttttctcaaaacttcccagtcggaggctggcgggaacccgagaggcgtgtctcgccagccacgcggaggggcgtggcctcattggcccgccccaccaactccagccaaactctaaaccccaggcggaggg

SEQ ID NO: 274 - DNA, 42 bp, Artificial Sequence
  Synthetic
cagttgtttatctttcgctccatcaaccaagtcacaattggt

SEQ ID NO: 275 - DNA, 29 bp, Artificial Sequence
  Synthetic
cgcgccgaggagttgggagggaatttctv

SEQ ID NO: 276 - DNA, 32 bp, Artificial Sequence
  Synthetic
atgacgtggcagacggttgggagggaatttcv

SEQ ID NO: 277 - DNA, 25 bp, Artificial Sequence
  Synthetic
ggcaggcttcagtttggccaggcca

SEQ ID NO: 278 - DNA, 30 bp, Artificial Sequence
  Synthetic
atgacgtggcagacctccatggtggtgctv

SEQ ID NO: 279 - DNA, 26 bp, Artificial Sequence
  Synthetic
cgcgccgaggttccatggtggtgctv

SEQ ID NO: 280 - DNA, 30 bp, Artificial Sequence
  Synthetic
agcatagtcccaggaatgaggtcccccaat

SEQ ID NO: 281 - DNA, 36 bp, Artificial Sequence
  Synthetic
atgacgtggcagacgttgctaagttttacatagggv

SEQ ID NO: 282 - DNA, 33 bp, Artificial Sequence
  Synthetic
cgcgccgaggattgctaagttttacataggggv

SEQ ID NO: 283 - DNA, 34 bp, Artificial Sequence
  Synthetic
ccaacacagatggagattatggcagacttgtttt

SEQ ID NO: 284 - DNA, 32 bp, Artificial Sequence
  Synthetic
atgacgtggcagacgttagaagagcagccctv

SEQ ID NO: 285 - DNA, 28 bp, Artificial Sequence
  Synthetic
cgcgccgaggcttagaagagcagccctv

SEQ ID NO: 286 - DNA, 30 bp, Artificial Sequence
  Synthetic
gctgcaccgcctcatcaatcccaacttctc

SEQ ID NO: 287 - DNA, 26 bp, Artificial Sequence
  Synthetic
cgcgccgaggtcggctatcaggacgv

SEQ ID NO: 288 - DNA, 30 bp, Artificial Sequence
  Synthetic
atgacgtggcagacacggctatcaggacgv

SEQ ID NO: 289 - DNA, 32 bp, Artificial Sequence
  Synthetic
gcatgacaggaagacagggtgtgaggttggat

SEQ ID NO: 290 - DNA, 27 bp, Artificial Sequence
  Synthetic
cgcgccgaggaggagagaggctgtagv

SEQ ID NO: 291 - DNA, 31 bp, Artificial Sequence
  Synthetic
atgacgtggcagacgggagagaggctgtagv

SEQ ID NO: 292 - DNA, 28 bp, Artificial Sequence
  Synthetic
actgctactgtctgtgctgtgctgggct

SEQ ID NO: 293 - DNA, 30 bp, Artificial Sequence
  Synthetic
atgacgtggcagacgcagagctggacaccv

SEQ ID NO: 294 - DNA, 26 bp, Artificial Sequence
  Synthetic
cgcgccgaggacagagctggacaccv

SEQ ID NO: 295 - DNA, 32 bp, Artificial Sequence
  Synthetic
ggtctctctggacagcacactgcaccaagtat

SEQ ID NO: 296 - DNA, 26 bp, Artificial Sequence
  Synthetic
cgcgccgaggagcccaccaaaaacgv

SEQ ID NO: 297 - DNA, 30 bp, Artificial Sequence
  Synthetic
atgacgtggcagacggcccaccaaaaacgv

SEQ ID NO: 298 - DNA, 42 bp, Artificial Sequence
  Synthetic
tcctatgcccaagttctctgatcatcctcaaaagaagacagt

SEQ ID NO: 299 - DNA, 27 bp, Artificial Sequence
  Synthetic
cgcgccgaggacttccatcccagaggv

SEQ ID NO: 300 - DNA, 31 bp, Artificial Sequence
  Synthetic
atgacgtggcagacccttccatcccagaggv

SEQ ID NO: 301 - DNA, 23 bp, Artificial Sequence
  Synthetic
ctgccrtgcccttcctggcccac

SEQ ID NO: 302 - DNA, 34 bp, Artificial Sequence
  Synthetic
cgcgccgaggtccctaaacctaaattcaaatctv

SEQ ID NO: 303 - DNA, 37 bp, Artificial Sequence
  Synthetic
atgacgtggcagacgccctaaacctaaattcaaatcv

SEQ ID NO: 304 - DNA, 31 bp, Artificial Sequence
  Synthetic
gctgcagagatgtgtcctcccacagaggagt

SEQ ID NO: 305 - DNA, 32 bp, Artificial Sequence
  Synthetic
atgacgtggcagacctgaaaccaccaaggagv

SEQ ID NO: 306 - DNA, 28 bp, Artificial Sequence
  Synthetic
cgcgccgaggatgaaaccaccaaggagv

SEQ ID NO: 307 - DNA, 33 bp, Artificial Sequence
  Synthetic
gcctctggtttctgtctactccaacgtccacgt

SEQ ID NO: 308 - DNA, 29 bp, Artificial Sequence
  Synthetic
cgcgccgaggcgcatagatacaggcatcv

SEQ ID NO: 309 - DNA, 33 bp, Artificial Sequence
  Synthetic
atgacgtggcagacggcatagatacaggcatcv

SEQ ID NO: 310 - DNA, 26 bp, Artificial Sequence
  Synthetic
gtccgtggggtttttgctgtgcggat

SEQ ID NO: 311 - DNA, 33 bp, Artificial Sequence
  Synthetic
atgacgtggcagacgtggaaagtacaaggctcv

SEQ ID NO: 312 - DNA, 29 bp, Artificial Sequence
  Synthetic
cgcgccgaggctggaaagtacaaggctcv

SEQ ID NO: 313 - DNA, 28 bp, Artificial Sequence
  Synthetic
cagaaggctgcagcctcacaatgcaggt

SEQ ID NO: 314 - DNA, 25 bp, Artificial Sequence
  Synthetic
cgcgccgaggatgactgggtccccv

SEQ ID NO: 315 - DNA, 29 bp, Artificial Sequence
  Synthetic
atgacgtggcagacgtgactgggtccccv

SEQ ID NO: 316 - DNA, 36 bp, Artificial Sequence
  Synthetic
cccaaatttgatccactgtaaccgtgcgtacacagt

SEQ ID NO: 317 - DNA, 31 bp, Artificial Sequence
  Synthetic
atgacgtggcagaccaccgttgcaacaacav

SEQ ID NO: 318 - DNA, 27 bp, Artificial Sequence
  Synthetic
cgcgccgaggaaccgttgcaacaacav

SEQ ID NO: 319 - DNA, 37 bp, Artificial Sequence
  Synthetic
gagagttgctcaaggtaacacagtggtaagtgacggt

SEQ ID NO: 320 - DNA, 31 bp, Artificial Sequence
  Synthetic
atgacgtggcagacggccaggaactagactv

SEQ ID NO: 321 - DNA, 28 bp, Artificial Sequence
  Synthetic
cgcgccgaggagccaggaactagactcv

SEQ ID NO: 322 - DNA, 30 bp, Artificial Sequence
  Synthetic
gcagtcagtagcagcagcttgagtggcaga

SEQ ID NO: 323 - DNA, 31 bp, Artificial Sequence
  Synthetic
atgacgtggcagaccggttctcaaacctggv

SEQ ID NO: 324 - DNA, 28 bp, Artificial Sequence
  Synthetic
cgcgccgaggtggttctcaaacctggav

SEQ ID NO: 325 - DNA, 30 bp, Artificial Sequence
  Synthetic
ccctctggaaggatggctmatttgcacaca

SEQ ID NO: 326 - DNA, 32 bp, Artificial Sequence
  Synthetic
atgacgtggcagacctgaggctttcctgatgv

SEQ ID NO: 327 - DNA, 29 bp, Artificial Sequence
  Synthetic
cgcgccgaggttgaggctttcctgatgav

SEQ ID NO: 328 - DNA, 24 bp, Artificial Sequence
  Synthetic
gcgaggccgagcccctcctagtgt

SEQ ID NO: 329 - DNA, 30 bp, Artificial Sequence
  Synthetic
atgacgtggcagacgttccggaccttgctv

SEQ ID NO: 330 - DNA, 26 bp, Artificial Sequence
  Synthetic
cgcgccgaggcttccggaccttgctv

SEQ ID NO: 331 - DNA, 39 bp, Artificial Sequence
  Synthetic
acaaaccttttagtttactctgcagttaatcccactgat

SEQ ID NO: 332 - DNA, 31 bp, Artificial Sequence
  Synthetic
atgacgtggcagacgaagtagtgggctccav

SEQ ID NO: 333 - DNA, 28 bp, Artificial Sequence
  Synthetic
cgcgccgaggaaagtagtgggctccaav

SEQ ID NO: 334 - DNA, 30 bp, Artificial Sequence
  Synthetic
tgtatgttggcctcctttgctgccctcact

SEQ ID NO: 335 - DNA, 33 bp, Artificial Sequence
  Synthetic
atgacgtggcagacgatctcttcctgtgacacv

SEQ ID NO: 336 - DNA, 29 bp, Artificial Sequence
  Synthetic
cgcgccgaggaatctcttcctgtgacacv

SEQ ID NO: 337 - DNA, 23 bp, Artificial Sequence
  Synthetic
gcccagagcgggagacagcgaca

SEQ ID NO: 338 - DNA, 34 bp, Artificial Sequence
  Synthetic
atgacgtggcagaccgacttggatatcaggtacv

SEQ ID NO: 339 - DNA, 31 bp, Artificial Sequence
  Synthetic
cgcgccgaggtgacttggatatcaggtactv

SEQ ID NO: 340 - DNA, 23 bp, Artificial Sequence
  Synthetic
tcgtggtccggcgcatggcttca

SEQ ID NO: 341 - DNA, 26 bp, Artificial Sequence
  Synthetic
cgcgccgaggtattgggtgccagcav

SEQ ID NO: 342 - DNA, 29 bp, Artificial Sequence
  Synthetic
atgacgtggcagaccattgggtgccagcv

SEQ ID NO: 343 - DNA, 38 bp, Artificial Sequence
  Synthetic
gtgatcattctgatggtgtggattgtgtcaggccttaa

SEQ ID NO: 344 - DNA, 30 bp, Artificial Sequence
  Synthetic
atgacgtggcagaccctccttcttgcccav

SEQ ID NO: 345 - DNA, 28 bp, Artificial Sequence
  Synthetic
cgcgccgaggtctccttcttgcccattv

SEQ ID NO: 346 - DNA, 28 bp, Artificial Sequence
  Synthetic
agcgacaccttcacgttgtcctggacct

SEQ ID NO: 347 - DNA, 30 bp, Artificial Sequence
  Synthetic
atgacgtggcagacgccgtctggttgttcv

SEQ ID NO: 348 - DNA, 27 bp, Artificial Sequence
  Synthetic
cgcgccgaggaccgtctggttgttccv

SEQ ID NO: 349 - DNA, 24 bp, Artificial Sequence
  Synthetic
gcggagccaaaggaccgagcaggc

SEQ ID NO: 350 - DNA, 28 bp, Artificial Sequence
  Synthetic
cgcgccgaggtttaatcccacagccagv

SEQ ID NO: 351 - DNA, 32 bp, Artificial Sequence
  Synthetic
atgacgtggcagacgttaatcccacagccagv

SEQ ID NO: 352 - DNA, 28 bp, Artificial Sequence
  Synthetic
gcgtgtcctccagggtgaacatgtccct

SEQ ID NO: 353 - DNA, 30 bp, Artificial Sequence
  Synthetic
atgacgtggcagacgctggacctgtgtgav

SEQ ID NO: 354 - DNA, 27 bp, Artificial Sequence
  Synthetic
cgcgccgaggactggacctgtgtgaav

SEQ ID NO: 355 - DNA, 30 bp, Artificial Sequence
  Synthetic
gcatttgattgcagagcagctccgagtcct

SEQ ID NO: 356 - DNA, 27 bp, Artificial Sequence
  Synthetic
cgcgccgaggatccagagcttcctgcv

SEQ ID NO: 357 - DNA, 31 bp, Artificial Sequence
  Synthetic
atgacgtggcagacgtccagagcttcctgcv

SEQ ID NO: 358 - DNA, 27 bp, Artificial Sequence
  Synthetic
gaacagcttcaccacggcggtcatgtt

SEQ ID NO: 359 - DNA, 30 bp, Artificial Sequence
  Synthetic
atgacgtggcagacgcttctgtcccctggv

SEQ ID NO: 360 - DNA, 26 bp, Artificial Sequence
  Synthetic
cgcgccgaggacttctgtcccctggv

SEQ ID NO: 361 - DNA, 34 bp, Artificial Sequence
  Synthetic
aacccatagttaagaacgtggggtgaggtaccgc

SEQ ID NO: 362 - DNA, 25 bp, Artificial Sequence
  Synthetic
cgcgccgaggtcctgccctttggcv

SEQ ID NO: 363 - DNA, 29 bp, Artificial Sequence
  Synthetic
atgacgtggcagacacctgccctttggcv

SEQ ID NO: 364 - DNA, 35 bp, Artificial Sequence
  Synthetic
gctggagtgtgcccaatgctatatgtcagttgagt

SEQ ID NO: 365 - DNA, 34 bp, Artificial Sequence
  Synthetic
atgacgtggcagacgttctaagacttggaagccv

SEQ ID NO: 366 - DNA, 30 bp, Artificial Sequence
  Synthetic
cgcgccgaggattctaagacttggaagccv

SEQ ID NO: 367 - DNA, 45 bp, Artificial Sequence
  Synthetic
gggaacaatcaccttttctctttgcctttcatactgctttagact

SEQ ID NO: 368 - DNA, 28 bp, Artificial Sequence
  Synthetic
cgcgccgaggacctcactgcttcctaav

SEQ ID NO: 369 - DNA, 32 bp, Artificial Sequence
  Synthetic
atgacgtggcagacccctcactgcttcctaav

SEQ ID NO: 370 - DNA, 39 bp, Artificial Sequence
  Synthetic
tttgttccggacatcatgtgtatcccaacctaccaaaat

SEQ ID NO: 371 - DNA, 30 bp, Artificial Sequence
  Synthetic
cgcgccgaggaagtcctttccaggtaaggv

SEQ ID NO: 372 - DNA, 33 bp, Artificial Sequence
  Synthetic
atgacgtggcagacgagtcctttccaggtaagv

SEQ ID NO: 373 - DNA, 40 bp, Artificial Sequence
  Synthetic
tttgtgcagtggttgatgaataccaacaggaacaggtaat

SEQ ID NO: 374 - DNA, 27 bp, Artificial Sequence
  Synthetic
cgcgccgaggaagtctaagcctggctv

SEQ ID NO: 375 - DNA, 31 bp, Artificial Sequence
  Synthetic
atgacgtggcagacgagtctaagcctggctv

SEQ ID NO: 376 - DNA, 30 bp, Artificial Sequence
  Synthetic
caggctcaggttgtggtgacactggtcaca

SEQ ID NO: 377 - DNA, 29 bp, Artificial Sequence
  Synthetic
cgcgccgaggtgtagagcttccacttctv

SEQ ID NO: 378 - DNA, 33 bp, Artificial Sequence
  Synthetic
atgacgtggcagaccgtagagcttccacttctv

SEQ ID NO: 379 - DNA, 27 bp, Artificial Sequence
  Synthetic
tggccctgtgactatggctctggcaca

SEQ ID NO: 380 - DNA, 30 bp, Artificial Sequence
  Synthetic
atgacgtggcagaccactagggtcctggcv

SEQ ID NO: 381 - DNA, 27 bp, Artificial Sequence
  Synthetic
cgcgccgaggtactagggtcctggccv

SEQ ID NO: 382 - DNA, 28 bp, Artificial Sequence
  Synthetic
cccatcctgaccaccatccgccgaatct

SEQ ID NO: 383 - DNA, 29 bp, Artificial Sequence
  Synthetic
cgcgccgaggagcctcttcaatagcagtv

SEQ ID NO: 384 - DNA, 32 bp, Artificial Sequence
  Synthetic
atgacgtggcagacggcctcttcaatagcagv

SEQ ID NO: 385 - DNA, 32 bp, Artificial Sequence
  Synthetic
ggagtcaagacccagatgtcccctgacttgtt

SEQ ID NO: 386 - DNA, 33 bp, Artificial Sequence
  Synthetic
atgacgtggcagacgtcacacaaggagtcttcv

SEQ ID NO: 387 - DNA, 30 bp, Artificial Sequence
  Synthetic
cgcgccgaggatcacacaaggagtcttcav

SEQ ID NO: 388 - DNA, 41 bp, Artificial Sequence
  Synthetic
cgactgtccagttaaatgcatcagaagtgttagcttctcct

SEQ ID NO: 389 - DNA, 38 bp, Artificial Sequence
  Synthetic
atgacgtggcagacggagttaaagtcattactgtagav

SEQ ID NO: 390 - DNA, 35 bp, Artificial Sequence
  Synthetic
cgcgccgaggagagttaaagtcattactgtagagv

SEQ ID NO: 391 - DNA, 25 bp, Artificial Sequence
  Synthetic
gagacacctcccactcgtccggcaa

SEQ ID NO: 392 - DNA, 28 bp, Artificial Sequence
  Synthetic
cgcgccgaggtgtacacagagcatggav

SEQ ID NO: 393 - DNA, 31 bp, Artificial Sequence
  Synthetic
atgacgtggcagaccgtacacagagcatggv

SEQ ID NO: 394 - DNA, 30 bp, Artificial Sequence
  Synthetic
ccaaggctgatgacattgttggccctgtgt

SEQ ID NO: 395 - DNA, 30 bp, Artificial Sequence
  Synthetic
cgcgccgaggacgcatgaaatctttgagav

SEQ ID NO: 396 - DNA, 33 bp, Artificial Sequence
  Synthetic
atgacgtggcagacgcgcatgaaatctttgagv

SEQ ID NO: 397 - DNA, 37 bp, Artificial Sequence
  Synthetic
cactcccaaattcaatattgacatattcccccgggca

SEQ ID NO: 398 - DNA, 26 bp, Artificial Sequence
  Synthetic
cgcgccgaggtcttgggctctggagv

SEQ ID NO: 399 - DNA, 30 bp, Artificial Sequence
  Synthetic
atgacgtggcagacccttgggctctggagv

SEQ ID NO: 400 - DNA, 22 bp, Artificial Sequence
  Synthetic
cgcctggcagaggaccctgcct

SEQ ID NO: 401 - DNA, 25 bp, Artificial Sequence
  Synthetic
cgcgccgaggaagcccaggtaccgv

SEQ ID NO: 402 - DNA, 29 bp, Artificial Sequence
  Synthetic
atgacgtggcagacgagcccaggtaccgv

SEQ ID NO: 403 - DNA, 28 bp, Artificial Sequence
  Synthetic
ccgtgcagagtggtgtgggcactttgaa

SEQ ID NO: 404 - DNA, 28 bp, Artificial Sequence
  Synthetic
cgcgccgaggtggtgttgccaaacttgv

SEQ ID NO: 405 - DNA, 31 bp, Artificial Sequence
  Synthetic
atgacgtggcagaccggtgttgccaaacttv

SEQ ID NO: 406 - DNA, 43 bp, Artificial Sequence
  Synthetic
ggttctcccgagaggtaaagaacaaagacttcaaagacacttc

SEQ ID NO: 407 - DNA, 28 bp, Artificial Sequence
  Synthetic
cgcgccgaggtcttcactggtcagctcv

SEQ ID NO: 408 - DNA, 30 bp, Artificial Sequence
  Synthetic
atgacgtggcagacgcttcactggtcagcv

SEQ ID NO: 409 - DNA, 43 bp, Artificial Sequence
  Synthetic
tgttgaacagtcttcaaggtgggatcgtaataatggcaaaagt

SEQ ID NO: 410 - DNA, 29 bp, Artificial Sequence
  Synthetic
cgcgccgaggacctcaccaagaatttggv

SEQ ID NO: 411 - DNA, 33 bp, Artificial Sequence
  Synthetic
atgacgtggcagacgcctcaccaagaatttggv

SEQ ID NO: 412 - DNA, 53 bp, Artificial Sequence
  Synthetic
cagcaatttcctcaaaagactttcctttggtttctggaactttaaaaaatgtt

SEQ ID NO: 413 - DNA, 31 bp, Artificial Sequence
  Synthetic
atgacgtggcagacgaacagggtaaaggccv

SEQ ID NO: 414 - DNA, 28 bp, Artificial Sequence
  Synthetic
cgcgccgaggaaacagggtaaaggccav

SEQ ID NO: 415 - DNA, 26 bp, Artificial Sequence
  Synthetic
ggcccagaagacccccctcggaatct

SEQ ID NO: 416 - DNA, 26 bp, Artificial Sequence
  Synthetic
cgcgccgaggagagcagggaggatgv

SEQ ID NO: 417 - DNA, 30 bp, Artificial Sequence
  Synthetic
atgacgtggcagacggagcagggaggatgv

SEQ ID NO: 418 - DNA, 29 bp, Artificial Sequence
  Synthetic
ctccatccgcatcggcctctatgactcct

SEQ ID NO: 419 - DNA, 28 bp, Artificial Sequence
  Synthetic
cgcgccgaggatcaagcaggtgtacacv

SEQ ID NO: 420 - DNA, 32 bp, Artificial Sequence
  Synthetic
atgacgtggcagacgtcaagcaggtgtacacv

SEQ ID NO: 421 - DNA, 29 bp, Artificial Sequence
  Synthetic
ggacactggtcggcaatcctcagcacagt

SEQ ID NO: 422 - DNA, 29 bp, Artificial Sequence
  Synthetic
atgacgtggcagacgacgccacttcccav

SEQ ID NO: 423 - DNA, 25 bp, Artificial Sequence
  Synthetic
cgcgccgaggcacgccacttcccav

SEQ ID NO: 424 - DNA, 25 bp, Artificial Sequence
  Synthetic
caccaggctgccttggccacagaaa

SEQ ID NO: 425 - DNA, 31 bp, Artificial Sequence
  Synthetic
cgcgccgaggtacttactgaaatgcccttgv

SEQ ID NO: 426 - DNA, 34 bp, Artificial Sequence
  Synthetic
atgacgtggcagaccacttactgaaatgcccttv

SEQ ID NO: 427 - DNA, 23 bp, Artificial Sequence
  Synthetic
gcctctgaccccatggcaggggt

SEQ ID NO: 428 - DNA, 29 bp, Artificial Sequence
  Synthetic
cgcgccgaggacagagtatttgagcagcv

SEQ ID NO: 429 - DNA, 33 bp, Artificial Sequence
  Synthetic
atgacgtggcagacgcagagtatttgagcagcv

SEQ ID NO: 430 - DNA, 20 bp, Artificial Sequence
  Synthetic
gctggggccccactgcccat

SEQ ID NO: 431 - DNA, 28 bp, Artificial Sequence
  Synthetic
cgcgccgaggatgtcaccttggatggcv

SEQ ID NO: 432 - DNA, 31 bp, Artificial Sequence
  Synthetic
atgacgtggcagacgtgtcaccttggatggv

SEQ ID NO: 433 - DNA, 35 bp, Artificial Sequence
  Synthetic
gttcatctttggttttgtgggcaacatgctggtct

SEQ ID NO: 434 - DNA, 33 bp, Artificial Sequence
  Synthetic
cgcgccgaggatcctcatcttaataaactgcav

SEQ ID NO: 435 - DNA, 37 bp, Artificial Sequence
  Synthetic
atgacgtggcagacgtcctcatcttaataaactgcav

SEQ ID NO: 436 - DNA, 30 bp, Artificial Sequence
  Synthetic
gctccactttcaacttgtccccctccagct

SEQ ID NO: 437 - DNA, 29 bp, Artificial Sequence
  Synthetic
atgacgtggcagacgtcacctgggaggcv

SEQ ID NO: 438 - DNA, 25 bp, Artificial Sequence
  Synthetic
cgcgccgaggatcacctgggaggcv

SEQ ID NO: 439 - DNA, 47 bp, Artificial Sequence
  Synthetic
gctctcttcatcatagtgaagtcttccttatccagcatcttgttcaa

SEQ ID NO: 440 - DNA, 31 bp, Artificial Sequence
  Synthetic
cgcgccgaggatgaacaagatgctggataav

SEQ ID NO: 441 - DNA, 34 bp, Artificial Sequence
  Synthetic
atgacgtggcagacgtgaacaagatgctggatav

SEQ ID NO: 442 - DNA, 46 bp, Artificial Sequence
  Synthetic
gtcctgtctctgcaaataatgatgctttcgaagtttcagttgaaca

SEQ ID NO: 443 - DNA, 26 bp, Artificial Sequence
  Synthetic
cgcgccgaggtgtccctcgcgaaaav

SEQ ID NO: 444 - DNA, 28 bp, Artificial Sequence
  Synthetic
atgacgtggcagaccgtccctcgcgaav

SEQ ID NO: 445 - DNA, 27 bp, Artificial Sequence
  Synthetic
gccatctccttctttgcgctcccagct

SEQ ID NO: 446 - DNA, 25 bp, Artificial Sequence
  Synthetic
cgcgccgaggagtaggtgccccgtv

SEQ ID NO: 447 - DNA, 28 bp, Artificial Sequence
  Synthetic
atgacgtggcagacggtaggtgccccgv

SEQ ID NO: 448 - DNA, 36 bp, Artificial Sequence
  Synthetic
ggcaggatgaaaacacttacgtcggaggatctctct

SEQ ID NO: 449 - DNA, 33 bp, Artificial Sequence
  Synthetic
atgacgtggcagacgttgctttctgacgtaccv

SEQ ID NO: 450 - DNA, 29 bp, Artificial Sequence
  Synthetic
cgcgccgaggattgctttctgacgtaccv

SEQ ID NO: 451 - DNA, 25 bp, Artificial Sequence
  Synthetic
ggagccaagcactgctcctcccact

SEQ ID NO: 452 - DNA, 29 bp, Artificial Sequence
  Synthetic
atgacgtggcagacggccagcatgaggcv

SEQ ID NO: 453 - DNA, 25 bp, Artificial Sequence
  Synthetic
cgcgccgaggagccagcatgaggcv

SEQ ID NO: 454 - DNA, 26 bp, Artificial Sequence
  Synthetic
cgcgttcagtccgtgcatgcggttct

SEQ ID NO: 455 - DNA, 26 bp, Artificial Sequence
  Synthetic
atgacgtggcagaccgctcccgggcv

SEQ ID NO: 456 - DNA, 22 bp, Artificial Sequence
  Synthetic
cgcgccgaggagctcccgggcv

SEQ ID NO: 457 - DNA, 39 bp, Artificial Sequence
  Synthetic
tggattatctaaatgaaacacagcagcttactccagagt

SEQ ID NO: 458 - DNA, 27 bp, Artificial Sequence
  Synthetic
cgcgccgaggatcaagtccaaggccav

SEQ ID NO: 459 - DNA, 31 bp, Artificial Sequence
  Synthetic
atgacgtggcagacgtcaagtccaaggccav

SEQ ID NO: 460 - DNA, 28 bp, Artificial Sequence
  Synthetic
cggcttgcagacaccgtggaaggttcta

SEQ ID NO: 461 - DNA, 29 bp, Artificial Sequence
  Synthetic
atgacgtggcagaccctgggactgctggv

SEQ ID NO: 462 - DNA, 25 bp, Artificial Sequence
  Synthetic
cgcgccgaggtctgggactgctggv

SEQ ID NO: 463 - DNA, 28 bp, Artificial Sequence
  Synthetic
ccatggggtcccatgctggcaggataaa

SEQ ID NO: 464 - DNA, 28 bp, Artificial Sequence
  Synthetic
cgcgccgaggtgggttcctgctctaacv

SEQ ID NO: 465 - DNA, 31 bp, Artificial Sequence
  Synthetic
atgacgtggcagaccgggttcctgctctaav

SEQ ID NO: 466 - DNA, 29 bp, Artificial Sequence
  Synthetic
ctccctgcaggtcacagtcaccaccatct

SEQ ID NO: 467 - DNA, 27 bp, Artificial Sequence
  Synthetic
cgcgccgaggagctatggggacaaggv

SEQ ID NO: 468 - DNA, 31 bp, Artificial Sequence
  Synthetic
atgacgtggcagacggctatggggacaaggv

SEQ ID NO: 469 - DNA, 23 bp, Artificial Sequence
  Synthetic
gcctggtccccaaggtaggggct

SEQ ID NO: 470 - DNA, 31 bp, Artificial Sequence
  Synthetic
atgacgtggcagaccaggtcgaggtagcagv

SEQ ID NO: 471 - DNA, 27 bp, Artificial Sequence
  Synthetic
cgcgccgaggaaggtcgaggtagcagv

SEQ ID NO: 472 - DNA, 26 bp, Artificial Sequence
  Synthetic
ccctaccttggggaccaggcccttga

SEQ ID NO: 473 - DNA, 29 bp, Artificial Sequence
  Synthetic
atgacgtggcagaccgctgtggaaccagv

SEQ ID NO: 474 - DNA, 26 bp, Artificial Sequence
  Synthetic
cgcgccgaggtgctgtggaaccaggv

SEQ ID NO: 475 - DNA, 43 bp, Artificial Sequence
  Synthetic
gggaggacaatcctgtggaaaggaaggtttttataatgtgttt

SEQ ID NO: 476 - DNA, 32 bp, Artificial Sequence
  Synthetic
atgacgtggcagacctgagaaggagggtgacv

SEQ ID NO: 477 - DNA, 28 bp, Artificial Sequence
  Synthetic
cgcgccgaggatgagaaggagggtgacv

SEQ ID NO: 478 - DNA, 36 bp, Artificial Sequence
  Synthetic
cctgtctgtatccagctttgcagttggtggaatgaa

SEQ ID NO: 479 - DNA, 33 bp, Artificial Sequence
  Synthetic
atgacgtggcagacctgcatcattctttggtgv

SEQ ID NO: 480 - DNA, 30 bp, Artificial Sequence
  Synthetic
cgcgccgaggttgcatcattctttggtggv

SEQ ID NO: 481 - DNA, 44 bp, Artificial Sequence
  Synthetic
ggaaagaagaaagagcagaggagggagattggaagtagaaatgt

SEQ ID NO: 482 - DNA, 29 bp, Artificial Sequence
  Synthetic
cgcgccgaggatgaatgcagaggcaaaav

SEQ ID NO: 483 - DNA, 32 bp, Artificial Sequence
  Synthetic
atgacgtggcagacctgaatgcagaggcaaav

SEQ ID NO: 484 - DNA, 55 bp, Artificial Sequence
  Synthetic
ggcacaaaccagataatattaagggaaatttggaattcagaaatgttcacttcat

SEQ ID NO: 485 - DNA, 32 bp, Artificial Sequence
  Synthetic
cgcgccgaggattacccatctcgaaaagaagv

SEQ ID NO: 486 - DNA, 35 bp, Artificial Sequence
  Synthetic
atgacgtggcagacgttacccatctcgaaaagaav

SEQ ID NO: 487 - DNA, 25 bp, Artificial Sequence
  Synthetic
tcccacccccactggactcaccact

SEQ ID NO: 488 - DNA, 31 bp, Artificial Sequence
  Synthetic
atgacgtggcagacgtgatggcaggtgaagv

SEQ ID NO: 489 - DNA, 27 bp, Artificial Sequence
  Synthetic
cgcgccgaggatgatggcaggtgaagv

SEQ ID NO: 490 - DNA, 26 bp, Artificial Sequence
  Synthetic
ggtgccggcaggcaagatagacagct

SEQ ID NO: 491 - DNA, 32 bp, Artificial Sequence
  Synthetic
atgacgtggcagacggtggagtagaagagctv

SEQ ID NO: 492 - DNA, 29 bp, Artificial Sequence
  Synthetic
cgcgccgaggagtggagtagaagagctgv

SEQ ID NO: 493 - DNA, 51 bp, Artificial Sequence
  Synthetic
ggttcagtccacataatgcattttctccttcaattctgaaaagtagctaac

SEQ ID NO: 494 - DNA, 30 bp, Artificial Sequence
  Synthetic
cgcgccgaggtgctcatttggtagtgaagv

SEQ ID NO: 495 - DNA, 34 bp, Artificial Sequence
  Synthetic
atgacgtggcagacggctcatttggtagtgaagv

SEQ ID NO: 496 - DNA, 24 bp, Artificial Sequence
  Synthetic
cggccactgagggagaaggccact

SEQ ID NO: 497 - DNA, 28 bp, Artificial Sequence
  Synthetic
atgacgtggcagacggacgtgatgccgv

SEQ ID NO: 498 - DNA, 25 bp, Artificial Sequence
  Synthetic
cgcgccgaggagacgtgatgccgcv

SEQ ID NO: 499 - DNA, 27 bp, Artificial Sequence
  Synthetic
gggtctccaccacggctttctggtggt

SEQ ID NO: 500 - DNA, 28 bp, Artificial Sequence
  Synthetic
atgacgtggcagacgccgcctcctcagv

SEQ ID NO: 501 - DNA, 24 bp, Artificial Sequence
  Synthetic
cgcgccgaggaccgcctcctcagv

SEQ ID NO: 502 - DNA, 26 bp, Artificial Sequence
  Synthetic
ctgagccatggtggccatgaagggga

SEQ ID NO: 503 - DNA, 27 bp, Artificial Sequence
  Synthetic
cgcgccgaggttctgggtcacatggcv

SEQ ID NO: 504 - DNA, 31 bp, Artificial Sequence
  Synthetic
atgacgtggcagacctctgggtcacatggcv

SEQ ID NO: 505 - DNA, 28 bp, Artificial Sequence
  Synthetic
ggtgccttctgatggggacgtgtctgct

SEQ ID NO: 506 - DNA, 30 bp, Artificial Sequence
  Synthetic
atgacgtggcagacgccaggagagaagggv

SEQ ID NO: 507 - DNA, 27 bp, Artificial Sequence
  Synthetic
cgcgccgaggaccaggagagaagggav

SEQ ID NO: 508 - DNA, 39 bp, Artificial Sequence
  Synthetic
ctgccttgtaccagcattacaaataatccagccacaaat

SEQ ID NO: 509 - DNA, 37 bp, Artificial Sequence
  Synthetic
atgacgtggcagacgtaaatgcttttcatttctgctv

SEQ ID NO: 510 - DNA, 33 bp, Artificial Sequence
  Synthetic
cgcgccgaggataaatgcttttcatttctgctv

SEQ ID NO: 511 - DNA, 33 bp, Artificial Sequence
  Synthetic
accaacgttgacatgcacgtccagaattgaggt

SEQ ID NO: 512 - DNA, 30 bp, Artificial Sequence
  Synthetic
atgacgtggcagacggaggttgcctttgcv

SEQ ID NO: 513 - DNA, 27 bp, Artificial Sequence
  Synthetic
cgcgccgaggagaggttgcctttgctv

SEQ ID NO: 514 - DNA, 32 bp, Artificial Sequence
  Synthetic
acactaaggtctcatcagggtttgggtggcat

SEQ ID NO: 515 - DNA, 32 bp, Artificial Sequence
  Synthetic
atgacgtggcagacgaaggaatggaaccaggv

SEQ ID NO: 516 - DNA, 28 bp, Artificial Sequence
  Synthetic
cgcgccgaggaaaggaatggaaccaggv

SEQ ID NO: 517 - DNA, 34 bp, Artificial Sequence
  Synthetic
cctagatgccctgcagaatccttcctgttacgga

SEQ ID NO: 518 - DNA, 28 bp, Artificial Sequence
  Synthetic
atgacgtggcagaccccccctccctgav

SEQ ID NO: 519 - DNA, 25 bp, Artificial Sequence
  Synthetic
cgcgccgaggtccccctccctgaav

SEQ ID NO: 520 - DNA, 21 bp, Artificial Sequence
  Synthetic
gcactggccacccgggacgct

SEQ ID NO: 521 - DNA, 25 bp, Artificial Sequence
  Synthetic
cgcgccgaggccccccaaggaaggv

SEQ ID NO: 522 - DNA, 29 bp, Artificial Sequence
  Synthetic
atgacgtggcagacgcccccaaggaaggv

SEQ ID NO: 523 - DNA, 27 bp, Artificial Sequence
  Synthetic
caggggtggatggtctctcactcccct

SEQ ID NO: 524 - DNA, 31 bp, Artificial Sequence
  Synthetic
atgacgtggcagacgggcctgtattcagtcv

SEQ ID NO: 525 - DNA, 27 bp, Artificial Sequence
  Synthetic
cgcgccgaggcggcctgtattcagtcv

SEQ ID NO: 526 - DNA, 31 bp, Artificial Sequence
  Synthetic
tggtgaccctgcccagatgtgaagtgtacat

SEQ ID NO: 527 - DNA, 27 bp, Artificial Sequence
  Synthetic
cgcgccgaggactctgtgttggggagv

SEQ ID NO: 528 - DNA, 30 bp, Artificial Sequence
  Synthetic
atgacgtggcagacgctctgtgttggggav

SEQ ID NO: 529 - DNA, 34 bp, Artificial Sequence
  Synthetic
ctcagccttaaaaagacctccagggcttgatgca

SEQ ID NO: 530 - DNA, 28 bp, Artificial Sequence
  Synthetic
cgcgccgaggtggtatgttgtcaggctv

SEQ ID NO: 531 - DNA, 31 bp, Artificial Sequence
  Synthetic
atgacgtggcagaccggtatgttgtcaggcv

SEQ ID NO: 532 - DNA, 33 bp, Artificial Sequence
  Synthetic
gctggaggaggctatgagaagtgaggtttgcat

SEQ ID NO: 533 - DNA, 28 bp, Artificial Sequence
  Synthetic
cgcgccgaggagaagaaagaggggcagv

SEQ ID NO: 534 - DNA, 31 bp, Artificial Sequence
  Synthetic
atgacgtggcagacggaagaaagaggggcav

SEQ ID NO: 535 - DNA, 38 bp, Artificial Sequence
  Synthetic
caatgggacgccatagagggcttttgagtagacatatt

SEQ ID NO: 536 - DNA, 30 bp, Artificial Sequence
  Synthetic
cgcgccgaggatcagtgtagaagggtgaav

SEQ ID NO: 537 - DNA, 33 bp, Artificial Sequence
  Synthetic
atgacgtggcagacgtcagtgtagaagggtgav

SEQ ID NO: 538 - DNA, 51 bp, Artificial Sequence
  Synthetic
acacatgtgtttcatttttagttttgttaaaaaaaaattctgacaaatcat

SEQ ID NO: 539 - DNA, 31 bp, Artificial Sequence
  Synthetic
atgacgtggcagacgaaatgggggttcaggv

SEQ ID NO: 540 - DNA, 28 bp, Artificial Sequence
  Synthetic
cgcgccgaggaaaatgggggttcaggav

SEQ ID NO: 541 - DNA, 29 bp, Artificial Sequence
  Synthetic
ggaggagagcaggcattgggctaaggagc

SEQ ID NO: 542 - DNA, 27 bp, Artificial Sequence
  Synthetic
atgacgtggcagacggggcagtgggcv

SEQ ID NO: 543 - DNA, 23 bp, Artificial Sequence
  Synthetic
cgcgccgaggtgggcagtgggcv

SEQ ID NO: 544 - DNA, 36 bp, Artificial Sequence
  Synthetic
gggacccattcctgtgtaatacaatgtctgcaccat

SEQ ID NO: 545 - DNA, 35 bp, Artificial Sequence
  Synthetic
cgcgccgaggatgctaataaagtcctattctcttv

SEQ ID NO: 546 - DNA, 38 bp, Artificial Sequence
  Synthetic
atgacgtggcagacgtgctaataaagtcctattctctv

SEQ ID NO: 547 - DNA, 28 bp, Artificial Sequence
  Synthetic
gacagaggcttctagaggggccagcagt

SEQ ID NO: 548 - DNA, 31 bp, Artificial Sequence
  Synthetic
atgacgtggcagacgtttggggagacttggv

SEQ ID NO: 549 - DNA, 28 bp, Artificial Sequence
  Synthetic
cgcgccgaggatttggggagacttgggv

SEQ ID NO: 550 - DNA, 26 bp, Artificial Sequence
  Synthetic
cctccaggctggccccctagattgct

SEQ ID NO: 551 - DNA, 29 bp, Artificial Sequence
  Synthetic
atgacgtggcagacgtctgctcctggcav

SEQ ID NO: 552 - DNA, 26 bp, Artificial Sequence
  Synthetic
cgcgccgaggatctgctcctggcatv

SEQ ID NO: 553 - DNA, 25 bp, Artificial Sequence
  Synthetic
tggactctgagccccacctgcgaga

SEQ ID NO: 554 - DNA, 33 bp, Artificial Sequence
  Synthetic
atgacgtggcagaccccctagaatcacagagav

SEQ ID NO: 555 - DNA, 30 bp, Artificial Sequence
  Synthetic
cgcgccgaggtccctagaatcacagagagv

SEQ ID NO: 556 - DNA, 24 bp, Artificial Sequence
  Synthetic
gggtgctgtccacactggctccct

SEQ ID NO: 557 - DNA, 29 bp, Artificial Sequence
  Synthetic
atgacgtggcagacgtcagggagcagccv

SEQ ID NO: 558 - DNA, 25 bp, Artificial Sequence
  Synthetic
cgcgccgaggatcagggagcagccv

SEQ ID NO: 559 - DNA, 31 bp, Artificial Sequence
  Synthetic
tcatgaacagcaaaggcgtgagcctcttcgt

SEQ ID NO: 560 - DNA, 29 bp, Artificial Sequence
  Synthetic
cgcgccgaggacatcatcaaccctgagav

SEQ ID NO: 561 - DNA, 32 bp, Artificial Sequence
  Synthetic
atgacgtggcagacgcatcatcaaccctgagv

SEQ ID NO: 562 - DNA, 22 bp, Artificial Sequence
  Synthetic
ggtggggctgggctgctagggt

SEQ ID NO: 563 - DNA, 32 bp, Artificial Sequence
  Synthetic
atgacgtggcagacgatccagatggcatgtgv

SEQ ID NO: 564 - DNA, 28 bp, Artificial Sequence
  Synthetic
cgcgccgaggaatccagatggcatgtgv

SEQ ID NO: 565 - DNA, 25 bp, Artificial Sequence
  Synthetic
cttgggccacggagggcaatgacct

SEQ ID NO: 566 - DNA, 24 bp, Artificial Sequence
  Synthetic
cgcgccgaggaagggtgcccctgv

SEQ ID NO: 567 - DNA, 28 bp, Artificial Sequence
  Synthetic
atgacgtggcagacgagggtgcccctgv

SEQ ID NO: 568 - DNA, 29 bp, Artificial Sequence
  Synthetic
agtgtggtgcagaaaacccttcaccccct

SEQ ID NO: 569 - DNA, 33 bp, Artificial Sequence
  Synthetic
atgacgtggcagacgtgtcaaaaggagctgacv

SEQ ID NO: 570 - DNA, 29 bp, Artificial Sequence
  Synthetic
cgcgccgaggatgtcaaaaggagctgacv

SEQ ID NO: 571 - DNA, 32 bp, Artificial Sequence
  Synthetic
ggtctctaccttgggtgctgttctctgcctct

SEQ ID NO: 572 - DNA, 29 bp, Artificial Sequence
  Synthetic
cgcgccgaggaggagctctctgtcaattv

SEQ ID NO: 573 - DNA, 31 bp, Artificial Sequence
  Synthetic
atgacgtggcagacgggagctctctgtcaav

SEQ ID NO: 574 - DNA, 31 bp, Artificial Sequence
  Synthetic
gtagggagaagtgcggcacagctaaaggagt

SEQ ID NO: 575 - DNA, 28 bp, Artificial Sequence
  Synthetic
atgacgtggcagacgagcgcctgcaccv

SEQ ID NO: 576 - DNA, 24 bp, Artificial Sequence
  Synthetic
cgcgccgaggaagcgcctgcaccv

SEQ ID NO: 577 - DNA, 43 bp, Artificial Sequence
  Synthetic
gctacgttttcttctcagttgaacagacacggtagaagactcc

SEQ ID NO: 578 - DNA, 33 bp, Artificial Sequence
  Synthetic
atgacgtggcagacgcccattttggaatgtgav

SEQ ID NO: 579 - DNA, 30 bp, Artificial Sequence
  Synthetic
cgcgccgaggtcccattttggaatgtgacv

SEQ ID NO: 580 - DNA, 26 bp, Artificial Sequence
  Synthetic
catgaccagggtgcaagcactgggct

SEQ ID NO: 581 - DNA, 33 bp, Artificial Sequence
  Synthetic
atgacgtggcagacgttgttctgtgggagtagv

SEQ ID NO: 582 - DNA, 30 bp, Artificial Sequence
  Synthetic
cgcgccgaggattgttctgtgggagtaggv

SEQ ID NO: 583 - DNA, 24 bp, Artificial Sequence
  Synthetic
ggagaggacaccagggtgggggtt

SEQ ID NO: 584 - DNA, 33 bp, Artificial Sequence
  Synthetic
atgacgtggcagacgaaggagacactactgccv

SEQ ID NO: 585 - DNA, 29 bp, Artificial Sequence
  Synthetic
cgcgccgaggaaaggagacactactgccv

SEQ ID NO: 586 - DNA, 31 bp, Artificial Sequence
  Synthetic
gcggagagacagggagatgacgccttaaagt

SEQ ID NO: 587 - DNA, 28 bp, Artificial Sequence
  Synthetic
atgacgtggcagacggtccgcgacatgv

SEQ ID NO: 588 - DNA, 25 bp, Artificial Sequence
  Synthetic
cgcgccgaggagtccgcgacatgcv

SEQ ID NO: 589 - DNA, 18 bp, Artificial Sequence
  Synthetic
cgggctacccatgggaca

SEQ ID NO: 590 - DNA, 28 bp, Artificial Sequence
  Synthetic
gtcttctggtattaagccgtaatttgca

SEQ ID NO: 591 - DNA, 26 bp, Artificial Sequence
  Synthetic
  misc_feature (5)..(5): n is a, c, g, or t
cagtntcaccagctgtggtagaacca

SEQ ID NO: 592 - DNA, 23 bp, Artificial Sequence
  Synthetic
aagaggagcatcactgtgaccca

SEQ ID NO: 593 - DNA, 31 bp, Artificial Sequence
  Synthetic
tcccttcctcagattatattcatcccagaaa

SEQ ID NO: 594 - DNA, 27 bp, Artificial Sequence
  Synthetic
tcaaccccctgacattatcttggatcc

SEQ ID NO: 595 - DNA, 30 bp, Artificial Sequence
  Synthetic
cactccccaacatctcatttatttttcaca

SEQ ID NO: 596 - DNA, 26 bp, Artificial Sequence
  Synthetic
gtcatggcaatcagttggtgaaagca

SEQ ID NO: 597 - DNA, 27 bp, Artificial Sequence
  Synthetic
tcttctttagactgccacgaggaaaaa

SEQ ID NO: 598 - DNA, 27 bp, Artificial Sequence
  Synthetic
gggagatgaggtactcactagttaaca

SEQ ID NO: 599 - DNA, 21 bp, Artificial Sequence
  Synthetic
ccctgaggaactcacgcagac

SEQ ID NO: 600 - DNA, 21 bp, Artificial Sequence
  Synthetic
gcacctctttgcgcaggaaga

SEQ ID NO: 601 - DNA, 20 bp, Artificial Sequence
  Synthetic
agtggtggcgctctcacaaa

SEQ ID NO: 602 - DNA, 30 bp, Artificial Sequence
  Synthetic
catttgttcaggcattacagtaaaatgcca

SEQ ID NO: 603 - DNA, 20 bp, Artificial Sequence
  Synthetic
cagggacaatcccatcccca

SEQ ID NO: 604 - DNA, 28 bp, Artificial Sequence
  Synthetic
gtgaattgtccatgatgagagccactac

SEQ ID NO: 605 - DNA, 20 bp, Artificial Sequence
  Synthetic
tgtcccagactgggtcagca

SEQ ID NO: 606 - DNA, 24 bp, Artificial Sequence
  Synthetic
gaatgaagaaggtactgtgggcca

SEQ ID NO: 607 - DNA, 27 bp, Artificial Sequence
  Synthetic
ctggaaacttctgccagattgttccta

SEQ ID NO: 608 - DNA, 24 bp, Artificial Sequence
  Synthetic
caaaggactccttgtcccctagaa

SEQ ID NO: 609 - DNA, 19 bp, Artificial Sequence
  Synthetic
ggtcctttgcgcaaaggca

SEQ ID NO: 610 - DNA, 20 bp, Artificial Sequence
  Synthetic
tttcagctcccctcctccca

SEQ ID NO: 611 - DNA, 22 bp, Artificial Sequence
  Synthetic
gtctgccttctcacagctttcc

SEQ ID NO: 612 - DNA, 26 bp, Artificial Sequence
  Synthetic
aggtgtaacttgagtctctgcctaac

SEQ ID NO: 613 - DNA, 16 bp, Artificial Sequence
  Synthetic
agctgctgggccagca

SEQ ID NO: 614 - DNA, 28 bp, Artificial Sequence
  Synthetic
caagctttaaaggcagtcgacattaaga

SEQ ID NO: 615 - DNA, 19 bp, Artificial Sequence
  Synthetic
gccagggatctagggctcc

SEQ ID NO: 616 - DNA, 18 bp, Artificial Sequence
  Synthetic
cccgtcctacccagacga

SEQ ID NO: 617 - DNA, 19 bp, Artificial Sequence
  Synthetic
tcctgctgacattccgcca

SEQ ID NO: 618 - DNA, 19 bp, Artificial Sequence
  Synthetic
ggtgcaccacccattccca

SEQ ID NO: 619 - DNA, 31 bp, Artificial Sequence
  Synthetic
gcaatcctggttaaggacttaagaattgtca

SEQ ID NO: 620 - DNA, 23 bp, Artificial Sequence
  Synthetic
acaaaccaacgccacttcctaac

SEQ ID NO: 621 - DNA, 17 bp, Artificial Sequence
  Synthetic
cagcgtggcagagtggc

SEQ ID NO: 622 - DNA, 26 bp, Artificial Sequence
  Synthetic
actctacgatgtgggcatttcagaga

SEQ ID NO: 623 - DNA, 18 bp, Artificial Sequence
  Synthetic
gcgcacctgtccgtagca

SEQ ID NO: 624 - DNA, 21 bp, Artificial Sequence
  Synthetic
gccccaacaagctctcactca

SEQ ID NO: 625 - DNA, 37 bp, Artificial Sequence
  Synthetic
gaagagggtgaatactataaaaatagacttaccttcc

SEQ ID NO: 626 - DNA, 31 bp, Artificial Sequence
  Synthetic
ttggctcaaatcgtgggataattctaagaaa

SEQ ID NO: 627 - DNA, 16 bp, Artificial Sequence
  Synthetic
ccaccgccacctccga

SEQ ID NO: 628 - DNA, 19 bp, Artificial Sequence
  Synthetic
gacccagcagaggtccgaa

SEQ ID NO: 629 - DNA, 40 bp, Artificial Sequence
  Synthetic
tttcaaaactatcaggacctttatcattcataggaaataa

SEQ ID NO: 630 - DNA, 30 bp, Artificial Sequence
  Synthetic
ttttaagatacctttccaagttctccctca

SEQ ID NO: 631 - DNA, 28 bp, Artificial Sequence
  Synthetic
gacttacattaggcagtgactcgatgaa

SEQ ID NO: 632 - DNA, 27 bp, Artificial Sequence
  Synthetic
cattgctgagaacattgcctatggaga

SEQ ID NO: 633 - DNA, 19 bp, Artificial Sequence
  Synthetic
ccctggagggagttgaccc

SEQ ID NO: 634 - DNA, 22 bp, Artificial Sequence
  Synthetic
gctcagtatgcctttcctcccc

SEQ ID NO: 635 - DNA, 17 bp, Artificial Sequence
  Synthetic
gcaacccgggaacggca

SEQ ID NO: 636 - DNA, 20 bp, Artificial Sequence
  Synthetic
tcgtccctttcctgcgtgac

SEQ ID NO: 637 - DNA, 23 bp, Artificial Sequence
  Synthetic
cctgctgaccaagaataaggccc

SEQ ID NO: 638 - DNA, 26 bp, Artificial Sequence
  Synthetic
cattggcatagcagttgatggcttcc

SEQ ID NO: 639 - DNA, 20 bp, Artificial Sequence
  Synthetic
catcagcatcggttctgccc

SEQ ID NO: 640 - DNA, 18 bp, Artificial Sequence
  Synthetic
ggcgatgctcagcccgaa

SEQ ID NO: 641 - DNA, 25 bp, Artificial Sequence
  Synthetic
tccctctgtttctttccctcacaga

SEQ ID NO: 642 - DNA, 25 bp, Artificial Sequence
  Synthetic
ggttgctgaagttgtgtgtgatcac

SEQ ID NO: 643 - DNA, 34 bp, Artificial Sequence
  Synthetic
ttccttagattcttctttggagcagaataaaaga

SEQ ID NO: 644 - DNA, 24 bp, Artificial Sequence
  Synthetic
cacaccatgtgaggtcatcagcaa

SEQ ID NO: 645 - DNA, 20 bp, Artificial Sequence
  Synthetic
gctccagggaggactcacca

SEQ ID NO: 646 - DNA, 22 bp, Artificial Sequence
  Synthetic
catgacctcagggatgcccaca

SEQ ID NO: 647 - DNA, 27 bp, Artificial Sequence
  Synthetic
ggcccgaacatagtaattcctggtaaa

SEQ ID NO: 648 - DNA, 17 bp, Artificial Sequence
  Synthetic
cgagtgggagaggccca

SEQ ID NO: 649 - DNA, 33 bp, Artificial Sequence
  Synthetic
tgtattacataaaccctactccaaacaaatgca

SEQ ID NO: 650 - DNA, 23 bp, Artificial Sequence
  Synthetic
gccagcaaacacatccaggaaca

SEQ ID NO: 651 - DNA, 28 bp, Artificial Sequence
  Synthetic
cgtttcttccatccttccaggatttgaa

SEQ ID NO: 652 - DNA, 26 bp, Artificial Sequence
  Synthetic
acctctctgtgctttctgtatcctca

SEQ ID NO: 653 - DNA, 22 bp, Artificial Sequence
  Synthetic
caggcaactggaactgaaaccc

SEQ ID NO: 654 - DNA, 22 bp, Artificial Sequence
  Synthetic
ctcagcttccaagggccattca

SEQ ID NO: 655 - DNA, 23 bp, Artificial Sequence
  Synthetic
ggctggacatccacttcatccac

SEQ ID NO: 656 - DNA, 19 bp, Artificial Sequence
  Synthetic
cgtagaaagagccgggcca

SEQ ID NO: 657 - DNA, 26 bp, Artificial Sequence
  Synthetic
agaatcggctgtctttgatgctgtaa

SEQ ID NO: 658 - DNA, 50 bp, Artificial Sequence
  Synthetic
cttatacttttagaaaaaagaagacattatcaagatattcatttttgtca

SEQ ID NO: 659 - DNA, 40 bp, Artificial Sequence
  Synthetic
acgagcataagaacttaataatgtcaagagaaattttaga

SEQ ID NO: 660 - DNA, 26 bp, Artificial Sequence
  Synthetic
tgactacagcaagtatctggactcca

SEQ ID NO: 661 - DNA, 18 bp, Artificial Sequence
  Synthetic
acaggtcccctccgctca

SEQ ID NO: 662 - DNA, 19 bp, Artificial Sequence
  Synthetic
ggccaggcacaggctgaaa

SEQ ID NO: 663 - DNA, 22 bp, Artificial Sequence
  Synthetic
ccagagccactacctttgtcca

SEQ ID NO: 664 - DNA, 32 bp, Artificial Sequence
  Synthetic
ggtgtcttaggagagaaaaaaaggtagaaaaa

SEQ ID NO: 665 - DNA, 22 bp, Artificial Sequence
  Synthetic
tgaccaaatgccctcaccttca

SEQ ID NO: 666 - DNA, 27 bp, Artificial Sequence
  Synthetic
cacaatatgctggatgactcctcagac

SEQ ID NO: 667 - DNA, 23 bp, Artificial Sequence
  Synthetic
tggttgttgaggtccctgaatcc

SEQ ID NO: 668 - DNA, 18 bp, Artificial Sequence
  Synthetic
cagggtccagctggagca

SEQ ID NO: 669 - DNA, 22 bp, Artificial Sequence
  Synthetic
gagaggcacccttcacaggaaa

SEQ ID NO: 670 - DNA, 32 bp, Artificial Sequence
  Synthetic
aagaaaatacttctttgagctcaactacgaac

SEQ ID NO: 671 - DNA, 17 bp, Artificial Sequence
  Synthetic
gaaggagccctgcccca

SEQ ID NO: 672 - DNA, 20 bp, Artificial Sequence
  Synthetic
agacccccaagggatcctcc

SEQ ID NO: 673 - DNA, 20 bp, Artificial Sequence
  Synthetic
ttcggctcctgccacatcaa

SEQ ID NO: 674 - DNA, 24 bp, Artificial Sequence
  Synthetic
tgcctctcacttcctctccttaca

SEQ ID NO: 675 - DNA, 24 bp, Artificial Sequence
  Synthetic
gtagccagactgatcactcccaaa

SEQ ID NO: 676 - DNA, 18 bp, Artificial Sequence
  Synthetic
gcagcagcagcagcagca

SEQ ID NO: 677 - DNA, 18 bp, Artificial Sequence
  Synthetic
gacgttgccgaagcccac

SEQ ID NO: 678 - DNA, 24 bp, Artificial Sequence
  Synthetic
agataggcaaaccctacaacagca

SEQ ID NO: 679 - DNA, 17 bp, Artificial Sequence
  Synthetic
cacaaagcgggccctcc

SEQ ID NO: 680 - DNA, 21 bp, Artificial Sequence
  Synthetic
ccccgaggaatacgtgctgac

SEQ ID NO: 681 - DNA, 22 bp, Artificial Sequence
  Synthetic
cagtaggctgtggtcctcatca

SEQ ID NO: 682 - DNA, 22 bp, Artificial Sequence
  Synthetic
gcccattgtagctgaggaggac

SEQ ID NO: 683 - DNA, 23 bp, Artificial Sequence
  Synthetic
gggtgctggtctcataggtctca

SEQ ID NO: 684 - DNA, 25 bp, Artificial Sequence
  Synthetic
agctcctacatcaccagtgagatcc

SEQ ID NO: 685 - DNA, 20 bp, Artificial Sequence
  Synthetic
ccggtttggttctcccgaga

SEQ ID NO: 686 - DNA, 22 bp, Artificial Sequence
  Synthetic
cccaaggaggagctgctgaaga

SEQ ID NO: 687 - DNA, 25 bp, Artificial Sequence
  Synthetic
gctcaggaacttcaggattgctacc

SEQ ID NO: 688 - DNA, 39 bp, Artificial Sequence
  Synthetic
agaaacaaagtagatgcatttgattcaagtttcttaaaa

SEQ ID NO: 689 - DNA, 25 bp, Artificial Sequence
  Synthetic
cagggtcctacacacaaatcagtca

SEQ ID NO: 690 - DNA, 24 bp, Artificial Sequence
  Synthetic
ccttctgtctcggtttcttctcca

SEQ ID NO: 691 - DNA, 22 bp, Artificial Sequence
  Synthetic
gttcagggacctggtcactcac

SEQ ID NO: 692 - DNA, 16 bp, Artificial Sequence
  Synthetic
ggcctgcagcgccaga

SEQ ID NO: 693 - DNA, 18 bp, Artificial Sequence
  Synthetic
agggcgttggcgttttcc

SEQ ID NO: 694 - DNA, 21 bp, Artificial Sequence
  Synthetic
agggcttgatggcctctcaga

SEQ ID NO: 695 - DNA, 24 bp, Artificial Sequence
  Synthetic
agcctacccatcttccattcctca

SEQ ID NO: 696 - DNA, 18 bp, Artificial Sequence
  Synthetic
gccaggcccccttaggac

SEQ ID NO: 697 - DNA, 22 bp, Artificial Sequence
  Synthetic
gcttctgcactgaaagggctca

SEQ ID NO: 698 - DNA, 19 bp, Artificial Sequence
  Synthetic
ccacatggcctaccctccc

SEQ ID NO: 699 - DNA, 17 bp, Artificial Sequence
  Synthetic
gcctgtgcccagcagca

SEQ ID NO: 700 - DNA, 17 bp, Artificial Sequence
  Synthetic
gcctaccctggcagccc

SEQ ID NO: 701 - DNA, 21 bp, Artificial Sequence
  Synthetic
caactcctgcctccgctctac

SEQ ID NO: 702 - DNA, 24 bp, Artificial Sequence
  Synthetic
gccaggttgagcaggtaaatgtca

SEQ ID NO: 703 - DNA, 23 bp, Artificial Sequence
  Synthetic
ccactctcctcctacactgtccc

SEQ ID NO: 704 - DNA, 19 bp, Artificial Sequence
  Synthetic
aggcccccatcgatctccc

SEQ ID NO: 705 - DNA, 29 bp, Artificial Sequence
  Synthetic
gcaaagatggctctcttcatcatagtgaa

SEQ ID NO: 706 - DNA, 21 bp, Artificial Sequence
  Synthetic
cgttctcacatgcatgccccc

SEQ ID NO: 707 - DNA, 20 bp, Artificial Sequence
  Synthetic
accaaaatcgaggtggccca

SEQ ID NO: 708 - DNA, 38 bp, Artificial Sequence
  Synthetic
catcagaaagaaaaatgaatctgcaacttcaatagtca

SEQ ID NO: 709 - DNA, 19 bp, Artificial Sequence
  Synthetic
caggaccccagctgtccaa

SEQ ID NO: 710 - DNA, 19 bp, Artificial Sequence
  Synthetic
cgggaagaccatcgcctcc

SEQ ID NO: 711 - DNA, 22 bp, Artificial Sequence
  Synthetic
gcacccctatgaagacccagaa

SEQ ID NO: 712 - DNA, 24 bp, Artificial Sequence
  Synthetic
caaaggtcacttcaggttgaggca

SEQ ID NO: 713 - DNA, 23 bp, Artificial Sequence
  Synthetic
tccagtgttgtagccaaactgca

SEQ ID NO: 714 - DNA, 23 bp, Artificial Sequence
  Synthetic
gtgtggtttgtttctccgcagaa

SEQ ID NO: 715 - DNA, 16 bp, Artificial Sequence
  Synthetic
ggcaccaccttgcgca

SEQ ID NO: 716 - DNA, 24 bp, Artificial Sequence
  Synthetic
cgcctggagcgttttaaattgaga

SEQ ID NO: 717 - DNA, 37 bp, Artificial Sequence
  Synthetic
gttgaaataacattcaagttttcccttactcaagtaa

SEQ ID NO: 718 - DNA, 28 bp, Artificial Sequence
  Synthetic
cagaatatggtcctctttgctcctaaca

SEQ ID NO: 719 - DNA, 18 bp, Artificial Sequence
  Synthetic
cagcagaaccacgggcac

SEQ ID NO: 720 - DNA, 24 bp, Artificial Sequence
  Synthetic
ccacacgcttccctctaattggac

SEQ ID NO: 721 - DNA, 22 bp, Artificial Sequence
  Synthetic
agctggagggcagtatcactca

SEQ ID NO: 722 - DNA, 21 bp, Artificial Sequence
  Synthetic
ggtggacaggaagcatgtccc

SEQ ID NO: 723 - DNA, 19 bp, Artificial Sequence
  Synthetic
gaggcgatggtcttcccga

SEQ ID NO: 724 - DNA, 19 bp, Artificial Sequence
  Synthetic
ccgtggctgaccactgtcc

SEQ ID NO: 725 - DNA, 19 bp, Artificial Sequence
  Synthetic
cgagctgcggccattctca

SEQ ID NO: 726 - DNA, 19 bp, Artificial Sequence
  Synthetic
acctggttccacagcgcaa

SEQ ID NO: 727 - DNA, 20 bp, Artificial Sequence
  Synthetic
gaccgtctgctacctcgacc

SEQ ID NO: 728 - DNA, 26 bp, Artificial Sequence
  Synthetic
caagattcccatttggaggaacggaa

SEQ ID NO: 729 - DNA, 35 bp, Artificial Sequence
  Synthetic
agcagctaataataaaccagtaatttgggatagac

SEQ ID NO: 730 - DNA, 19 bp, Artificial Sequence
  Synthetic
gtgactccgagggcagaca

SEQ ID NO: 731 - DNA, 36 bp, Artificial Sequence
  Synthetic
gtttatgcttatttatgaaatttgcctaccttccaa

SEQ ID NO: 732 - DNA, 23 bp, Artificial Sequence
  Synthetic
ggcagctgctcaactaatcacca

SEQ ID NO: 733 - DNA, 24 bp, Artificial Sequence
  Synthetic
ctgtctgctcctctctcatcatcc

SEQ ID NO: 734 - DNA, 24 bp, Artificial Sequence
  Synthetic
ggacagaagcaagtctgcagatca

SEQ ID NO: 735 - DNA, 37 bp, Artificial Sequence
  Synthetic
tctacaagaaaacatcagaaactcttcattcaataga

SEQ ID NO: 736 - DNA, 29 bp, Artificial Sequence
  Synthetic
gaagccaagtattgacagctattcgaaga

SEQ ID NO: 737 - DNA, 20 bp, Artificial Sequence
  Synthetic
gggaagggtcaggaaagcca

SEQ ID NO: 738 - DNA, 23 bp, Artificial Sequence
  Synthetic
cgagagcggattgagttcctcaa

SEQ ID NO: 739 - DNA, 18 bp, Artificial Sequence
  Synthetic
gagccacgagctcccaca

SEQ ID NO: 740 - DNA, 22 bp, Artificial Sequence
  Synthetic
ggtagccctttaaaaggcctcc

SEQ ID NO: 741 - DNA, 30 bp, Artificial Sequence
  Synthetic
tgacatgttcgaaacctgtccataaagtaa

SEQ ID NO: 742 - DNA, 34 bp, Artificial Sequence
  Synthetic
ggaaagaaaagcttttgttcagagctttagaaaa

SEQ ID NO: 743 - DNA, 21 bp, Artificial Sequence
  Synthetic
gctgatctgcttctcccacga

SEQ ID NO: 744 - DNA, 19 bp, Artificial Sequence
  Synthetic
agtcgtcgtagccagcgaa

SEQ ID NO: 745 - DNA, 21 bp, Artificial Sequence
  Synthetic
gcagggctccttactgcagaa

SEQ ID NO: 746 - DNA, 21 bp, Artificial Sequence
  Synthetic
cacgccacccatcctcaaaga

SEQ ID NO: 747 - DNA, 16 bp, Artificial Sequence
  Synthetic
gcacagggcgctcacc

SEQ ID NO: 748 - DNA, 18 bp, Artificial Sequence
  Synthetic
cctaccagcagccgctca

SEQ ID NO: 749 - DNA, 22 bp, Artificial Sequence
  Synthetic
aggctcccttagatgcctgaca

SEQ ID NO: 750 - DNA, 17 bp, Artificial Sequence
  Synthetic
cagggcgctgacaccca

SEQ ID NO: 751 - DNA, 27 bp, Artificial Sequence
  Synthetic
atttctcctctgtgtcttgaagggaac

SEQ ID NO: 752 - DNA, 18 bp, Artificial Sequence
  Synthetic
ctgcccccctcaccctac

SEQ ID NO: 753 - DNA, 23 bp, Artificial Sequence
  Synthetic
cctttcatttttcccggcacaga

SEQ ID NO: 754 - DNA, 21 bp, Artificial Sequence
  Synthetic
gggaacttctttcccctcgca

SEQ ID NO: 755 - DNA, 25 bp, Artificial Sequence
  Synthetic
ggagtttctgtcctgggaggaaaaa

SEQ ID NO: 756 - DNA, 20 bp, Artificial Sequence
  Synthetic
aacactcgtgaagctggcca

SEQ ID NO: 757 - DNA, 18 bp, Artificial Sequence
  Synthetic
ggccacagagcctggaga

SEQ ID NO: 758 - DNA, 19 bp, Artificial Sequence
  Synthetic
cggcttgcctgtgcagtca

SEQ ID NO: 759 - DNA, 19 bp, Artificial Sequence
  Synthetic
gccagcccccttcctttcc

SEQ ID NO: 760 - DNA, 22 bp, Artificial Sequence
  Synthetic
acactgccaggagacacagaac

SEQ ID NO: 761 - DNA, 23 bp, Artificial Sequence
  Synthetic
ggagcagatcctggcaaagatcc

SEQ ID NO: 762 - DNA, 22 bp, Artificial Sequence
  Synthetic
cgtactgcacaaacttgctgca

SEQ ID NO: 763 - DNA, 32 bp, Artificial Sequence
  Synthetic
ggtgtaggtagagataagaagagtgatactca

SEQ ID NO: 764 - DNA, 19 bp, Artificial Sequence
  Synthetic
gctggtgacttgccccaga

SEQ ID NO: 765 - DNA, 24 bp, Artificial Sequence
  Synthetic
tgtcataatgcagtgggattgcca

SEQ ID NO: 766 - DNA, 22 bp, Artificial Sequence
  Synthetic
caagctggcaatggtggacaca

SEQ ID NO: 767 - DNA, 26 bp, Artificial Sequence
  Synthetic
ttttaactctctgctgttccctcacc

SEQ ID NO: 768 - DNA, 25 bp, Artificial Sequence
  Synthetic
actgacagggaatctccagaagtca

SEQ ID NO: 769 - DNA, 27 bp, Artificial Sequence
  Synthetic
gacctgatttgattgagagccttgaac

SEQ ID NO: 770 - DNA, 29 bp, Artificial Sequence
  Synthetic
acaagccctggactagatgatttctaaga

SEQ ID NO: 771 - DNA, 23 bp, Artificial Sequence
  Synthetic
ggtgatgcaaaagatggaagcca

SEQ ID NO: 772 - DNA, 26 bp, Artificial Sequence
  Synthetic
ccattttggaatgtgaccgtctgtcc

SEQ ID NO: 773 - DNA, 20 bp, Artificial Sequence
  Synthetic
gagagggtcatgcagtggca

SEQ ID NO: 774 - DNA, 21 bp, Artificial Sequence
  Synthetic
tccggtgctccatggatgaca

SEQ ID NO: 775 - DNA, 25 bp, Artificial Sequence
  Synthetic
gccatactgcagcactttaaaggac

SEQ ID NO: 776 - DNA, 28 bp, Artificial Sequence
  Synthetic
ctgctgtgatttatctgctgaaagctca

SEQ ID NO: 777 - DNA, 23 bp, Artificial Sequence
  Synthetic
catctaactgctccccagtcaca

SEQ ID NO: 778 - DNA, 19 bp, Artificial Sequence
  Synthetic
gccctcggtcctccaggaa

SEQ ID NO: 779 - DNA, 19 bp, Artificial Sequence
  Synthetic
ccacccacccaggacacac

SEQ ID NO: 780 - DNA, 18 bp, Artificial Sequence
  Synthetic
ccccaacggccaggcaaa

SEQ ID NO: 781 - DNA, 31 bp, Artificial Sequence
  Synthetic
ggacaaatgttctgggtctctaatattccaa

SEQ ID NO: 782 - DNA, 17 bp, Artificial Sequence
  Synthetic
gggtgggacggagtccc

SEQ ID NO: 783 - DNA, 24 bp, Artificial Sequence
  Synthetic
gtttgccttaccttggaagtggac

SEQ ID NO: 784 - DNA, 27 bp, Artificial Sequence
  Synthetic
tgctgagaagattgacaggttcatgca

SEQ ID NO: 785 - DNA, 17 bp, Artificial Sequence
  Synthetic
ggcccagccactgacca

SEQ ID NO: 786 - DNA, 25 bp, Artificial Sequence
  Synthetic
cttcttggctgttgtttctgttccc

SEQ ID NO: 787 - DNA, 16 bp, Artificial Sequence
  Synthetic
cccgactgtgccgcca

SEQ ID NO: 788 - DNA, 23 bp, Artificial Sequence
  Synthetic
gccctttttccaggtctgacaac

SEQ ID NO: 789 - DNA, 23 bp, Artificial Sequence
  Synthetic
cctctcaatgggtcacttggcaa

SEQ ID NO: 790 - DNA, 28 bp, Artificial Sequence
  Synthetic
gtccaaatttctgttgggttcagtgaaa

SEQ ID NO: 791 - DNA, 20 bp, Artificial Sequence
  Synthetic
gaagggccaatagccctccc

SEQ ID NO: 792 - DNA, 21 bp, Artificial Sequence
  Synthetic
gccccagccaagaaaggtcaa

SEQ ID NO: 793 - DNA, 22 bp, Artificial Sequence
  Synthetic
ttctcctggcctgtagggagaa

SEQ ID NO: 794 - DNA, 21 bp, Artificial Sequence
  Synthetic
ccctcgtcacttcctctgtcc

SEQ ID NO: 795 - DNA, 20 bp, Artificial Sequence
  Synthetic
gtgtctccttcacccccacc

SEQ ID NO: 796 - DNA, 29 bp, Artificial Sequence
  Synthetic
actttccccttttcatgccttattctgac

SEQ ID NO: 797 - DNA, 16 bp, Artificial Sequence
  Synthetic
cgaccaggcaggccac

SEQ ID NO: 798 - DNA, 19 bp, Artificial Sequence
  Synthetic
gtcctgctcacacagcccc

SEQ ID NO: 799 - DNA, 23 bp, Artificial Sequence
  Synthetic
ggtgatgcaaaagatggaagcca

SEQ ID NO: 800 - DNA, 26 bp, Artificial Sequence
  Synthetic
ccattttggaatgtgaccgtctgtcc

SEQ ID NO: 801 - DNA, 19 bp, Artificial Sequence
  Synthetic
cggaatttcctggccccca

SEQ ID NO: 802 - DNA, 16 bp, Artificial Sequence
  Synthetic
gtcccggcctgtccca

SEQ ID NO: 803 - DNA, 19 bp, Artificial Sequence
  Synthetic
ctgggctgggagcagcctc

SEQ ID NO: 804 - DNA, 23 bp, Artificial Sequence
  Synthetic
cactcgctggcctgtttcatgtc

SEQ ID NO: 805 - DNA, 22 bp, Artificial Sequence
  Synthetic
ctggaatccggtgtcgaagtgg

SEQ ID NO: 806 - DNA, 20 bp, Artificial Sequence
  Synthetic
ctcggcccctgcactgtttc

SEQ ID NO: 807 - DNA, 22 bp, Artificial Sequence
  Synthetic
gaggcaagaaggagtgtcaggg

SEQ ID NO: 808 - DNA, 23 bp, Artificial Sequence
  Synthetic
agtcctgtggtgaggtgacgagg

SEQ ID NO: 809 - DNA, 15 bp, Artificial Sequence
  Synthetic
ggtagtgaggcaggt

SEQ ID NO: 810 - DNA, 16 bp, Artificial Sequence
  Synthetic
gcttctggtaggggag

SEQ ID NO: 811 - DNA, 19 bp, Artificial Sequence
  Synthetic
aaataggactaggacctgt

SEQ ID NO: 812 - DNA, 15 bp, Artificial Sequence
  Synthetic
gggtcccacggaaat

SEQ ID NO: 813 - DNA, 12 bp, Artificial Sequence
  Synthetic
catggccacgcg

SEQ ID NO: 814 - DNA, 13 bp, Artificial Sequence
  Synthetic
ccggcacctctcg

SEQ ID NO: 815 - DNA, 14 bp, Artificial Sequence
  Synthetic
ccgtcctcctgcat

SEQ ID NO: 816 - DNA, 17 bp, Artificial Sequence
  Synthetic
cactctcaccttctcca

SEQ ID NO: 817 - DNA, 17 bp, Artificial Sequence
  Synthetic
gttctgtcccgagtatg

SEQ ID NO: 818 - DNA, 16 bp, Artificial Sequence
  Synthetic
tgcactgtttcccaga

SEQ ID NO: 819 - DNA, 17 bp, Artificial Sequence
  Synthetic
ctgacctcctccaacat

SEQ ID NO: 820 - DNA, 15 bp, Artificial Sequence
  Synthetic
gggctatcaccaggt

SEQ ID NO: 821 - DNA, 17 bp, Artificial Sequence
  Synthetic
ctgacctcctccaacat

SEQ ID NO: 822 - DNA, 15 bp, Artificial Sequence
  Synthetic
gggctatcaccaggt

SEQ ID NO: 823 - DNA, 31 bp, Artificial Sequence
  Synthetic
taatctgtaagagcagatccctggacagrcc

SEQ ID NO: 824 - DNA, 33 bp, Artificial Sequence
  Synthetic
aacgaggcgcacgaggaatacaggtattttgtc

SEQ ID NO: 825 - DNA, 33 bp, Artificial Sequence
  Synthetic
aacgaggcgcacaaggaatacaggtattttgtc

SEQ ID NO: 826 - DNA, 39 bp, Artificial Sequence
  Synthetic
  misc_feature (4)..(4): The residue at this position is linked to an abasic linker with a group called a Quencher on it..
cctcgtctcggttttccgagacgagggtgcgcctcgttt

SEQ ID NO: 827 - DNA, 56 bp, Artificial Sequence
  Synthetic
aaggacaaaatacctgtattcctcgcctgtccagggatctgctcttacagattaga

SEQ ID NO: 828 - DNA, 56 bp, Artificial Sequence
  Synthetic
aaggacaaaatacctgtattccttgcctgtccagggatctgctcttacagattaga

SEQ ID NO: 829 - DNA, 30 bp, Artificial Sequence
  Synthetic
tatggttcccaataaaagtgactctcagct

SEQ ID NO: 830 - DNA, 28 bp, Artificial Sequence
  Synthetic
aacgaggcgcacgagcctcaatgctccc

SEQ ID NO: 831 - DNA, 28 bp, Artificial Sequence
  Synthetic
aacgaggcgcacaagcctcaatgctccc

SEQ ID NO: 832 - DNA, 39 bp, Artificial Sequence; identical to SEQ ID NO: 826
  Synthetic
  misc_feature (4)..(4): The residue at this position is linked to an abasic linker with a group called a Quencher on it..

SEQ ID NO: 833 - DNA, 71 bp, Artificial Sequence
  Synthetic
tagcactgggagcattgaggctcgctgagagtcacttttattgggaaccatagttttagaaacacaaaaat

SEQ ID NO: 834 - DNA, 71 bp, Artificial Sequence
  Synthetic
tagcactgggagcattgaggcttgctgagagtcacttttattgggaaccatagttttagaaacacaaaaat

SEQ ID NO: 835 - DNA, 30 bp, Artificial Sequence
  Synthetic
caaagaaaagctgcgtgatgatgaaatcgc

SEQ ID NO: 836 - DNA, 26 bp, Artificial Sequence
  Synthetic
aacgaggcgcacgctcccgcagacac

SEQ ID NO: 837 - DNA, 27 bp, Artificial Sequence
  Synthetic
aacgaggcgcacactcccgcagacacc

SEQ ID NO: 838 - DNA, 39 bp, Artificial Sequence; identical to SEQ ID NO: 826
  Synthetic
  misc_feature (4)..(4): The residue at this position is linked to an abasic linker with a group called a Quencher on it..

SEQ ID NO: 839 - DNA, 50 bp, Artificial Sequence
  Synthetic
gaaggtgtctgcgggagccgatttcatcatcacgcagcttttctttgagg

SEQ ID NO: 840 - DNA, 50 bp, Artificial Sequence
  Synthetic
gaaggtgtctgcgggagtcgatttcatcatcacgcagcttttctttgagg

SEQ ID NO: 841 - DNA, 37 bp, Artificial Sequence
  Synthetic
cccgagaggtaaagaacaaagacttcaaagacactta

SEQ ID NO: 842 - DNA, 32 bp, Artificial Sequence
  Synthetic
aacgaacgcgcagtcttcactggtcagctatt

SEQ ID NO: 843 - DNA, 32 bp, Artificial Sequence
  Synthetic
aacgaacgcgcaggcttcactggtcagctatt

SEQ ID NO: 844 - DNA, 40 bp, Artificial Sequence
  Synthetic
  misc_feature (38)..(40): The residues at this position are 2′-O-methyl U.
acgcgtctcggttttccgagacgcgtctgcgcgttcguuu

SEQ ID NO: 845 - DNA, 58 bp, Artificial Sequence
  Synthetic
ggagctgaccagtgaagaaagtgtctttgaagtctttgttctttacctctcgggattt

SEQ ID NO: 846 - DNA, 58 bp, Artificial Sequence
  Synthetic
ggagctgaccagtgaagcaagtgtctttgaagtctttgttctttacctctcgggattt

SEQ ID NO: 847 - DNA, 28 bp, Artificial Sequence
  Synthetic
cccctggggaagagcagagatatacgtc

SEQ ID NO: 848 - DNA, 29 bp, Artificial Sequence
  Synthetic
aacgaacgcgcaggccaggtggagcattt

SEQ ID NO: 849 - DNA, 27 bp, Artificial Sequence
  Synthetic
aacgaacgcgcagaccaggtggagcac

SEQ ID NO: 850 - DNA, 40 bp, Artificial Sequence
  Synthetic
  misc_feature (38)..(40): The residues at this position are 2′-O-methyl U.
ctccgtctcggttttccgagacggagctgcgcgttcguuu

SEQ ID NO: 851 - DNA, 39 bp, Artificial Sequence
  Synthetic
ggtgctccacctggcacgtatatctctgctcttccccag

SEQ ID NO: 852 - DNA, 39 bp, Artificial Sequence
  Synthetic
ggtgctccacctggtacgtatatctctgctcttccccag

SEQ ID NO: 853 - DNA, 24 bp, Artificial Sequence
  Synthetic
gggctccacacggcgactctcatt

SEQ ID NO: 854 - DNA, 35 bp, Artificial Sequence
  Synthetic
aagcacgcagcacgatcatagaacacgaacagttt

SEQ ID NO: 855 - DNA, 35 bp, Artificial Sequence
  Synthetic
aagcacgcagcaccatcatagaacacgaacagttt

SEQ ID NO: 856 - DNA, 40 bp, Artificial Sequence
  Synthetic
  misc_feature (38)..(40): The residues at this position are 2′-O-methyl U.
acgcgtctcggttttccgagacgcgtgtgctgcgtgcuuu

SEQ ID NO: 857 - DNA, 46 bp, Artificial Sequence
  Synthetic
agctgttcgtgttctatgatcatgagagtcgccgtgtggagccccg

SEQ ID NO: 858 - DNA, 46 bp, Artificial Sequence
  Synthetic
agctgttcgtgttctatgatgatgagagtcgccgtgtggagccccg

SEQ ID NO: 859 - DNA, 33 bp, Artificial Sequence
  Synthetic
tctaatctgtaagagcagatccctggacaggcc

SEQ ID NO: 860 - DNA, 33 bp, Artificial Sequence
  Synthetic
tctaatctgtaagagcagatccctggacagacc

SEQ ID NO: 861 - DNA, 32 bp, Artificial Sequence
  Synthetic
cgcgaggccggaggaatacaggtattttgtcc

SEQ ID NO: 862 - DNA, 33 bp, Artificial Sequence
  Synthetic
aggccacggacgaaggaatacaggtattttgtc

SEQ ID NO: 863 - DNA, 33 bp, Artificial Sequence
  Synthetic
  misc_feature (3)..(3): The residue at this position is linked to a Z28 quencher.
tctagccggttttccggctgagacggcctcgcg

SEQ ID NO: 864 - DNA, 35 bp, Artificial Sequence
  Synthetic
  misc_feature (3)..(3): The residue at this position is linked to a Z28 quencher.
tctagccggttttccggctgagacgtccgtggcct

SEQ ID NO: 865 - DNA, 66 bp, Artificial Sequence
  Synthetic
tcaaggacaaaatacctgtattcctcgcctgtccagggatctgctcttacagattagaagtgattt

SEQ ID NO: 866 - DNA, 66 bp, Artificial Sequence
  Synthetic
tcaaggacaaaatacctgtattcctggcctgtccagggatctgctcttacagattagaagtgattt

SEQ ID NO: 867 - DNA, 30 bp, Artificial Sequence; identical to SEQ ID NO: 829
  Synthetic

SEQ ID NO: 868 - DNA, 30 bp, Artificial Sequence
  Synthetic
acggacgcggaggagcctcaatgctaccag

SEQ ID NO: 869 - DNA, 27 bp, Artificial Sequence
  Synthetic
aggccacggacgaagcctcaatgctcc

SEQ ID NO: 870 - DNA, 35 bp, Artificial Sequence
  Synthetic
  misc_feature (3)..(3): The residue at this position is linked to an abasic linker with a group called a Quencher on it..
tcttcggccttttggccgagagactccgcgtccgt

SEQ ID NO: 871 - DNA, 35 bp, Artificial Sequence; identical to SEQ ID NO: 864
  Synthetic
  misc_feature (3)..(3): The residue at this position is linked to an abasic linker with a group called a Quencher on it..

SEQ ID NO: 872 - DNA, 71 bp, Artificial Sequence; identical to SEQ ID NO: 833
  Synthetic

SEQ ID NO: 873 - DNA, 71 bp, Artificial Sequence; identical to SEQ ID NO: 834
  Synthetic

SEQ ID NO: 874 - DNA, 30 bp, Artificial Sequence; identical to SEQ ID NO: 835
  Synthetic

SEQ ID NO: 875 - DNA, 30 bp, Artificial Sequence
  Synthetic
caaagaaaagctgcgtgatgatgaaattgc

SEQ ID NO: 876 - DNA, 26 bp, Artificial Sequence
  Synthetic
acggacgcggaggctcccgcagacac

SEQ ID NO: 877 - DNA, 26 bp, Artificial Sequence
  Synthetic
aggccacggacgactcccgcagacac

SEQ ID NO: 878 - DNA, 35 bp, Artificial Sequence
  Synthetic
  misc_feature (3)..(3): The residue at this position is linked to an abasic linker with a group called a Quencher on it..
tctagccggttttccggctgagactccgcgtccgt

SEQ ID NO: 879 - DNA, 35 bp, Artificial Sequence
  Synthetic
  misc_feature (3)..(3): The residue at this position is linked to an abasic linker with a group called a Quencher on it..
tcttcggccttttggccgagagacgtccgtggcct

SEQ ID NO: 880 - DNA, 50 bp, Artificial Sequence; identical to SEQ ID NO: 839
  Synthetic

SEQ ID NO: 881 - DNA, 28 bp, Artificial Sequence
  Synthetic
cgcgccgaggtgtctctgatgtacaacg

SEQ ID NO: 882 - DNA, 29 bp, Artificial Sequence
  Synthetic
tccgcgcgtcccgtctctgatgtacaacg

SEQ ID NO: 883 - DNA, 35 bp, Artificial Sequence
  Synthetic
ggcacagggtacgtcttcaaggtgtaaaatgctca

SEQ ID NO: 884 - DNA, 62 bp, Artificial Sequence
  Synthetic
cgcctcgttgtacatcagagacagagcattttacaccttgaagacgtaccctgtgccatttt

SEQ ID NO: 885 - DNA, 62 bp, Artificial Sequence
  Synthetic
cgcctcgttgtacatcagagacggagcattttacaccttgaagacgtaccctgtgccatttt

SEQ ID NO: 886 - DNA, 12 bp, Artificial Sequence
  Synthetic
tccgcgcgtccc

SEQ ID NO: 887 - DNA, 34 bp, Artificial Sequence
  Synthetic
tcttcggccttttggccgagagaggacgcgcgga

SEQ ID NO: 888 - DNA, 11 bp, Artificial Sequence
  Synthetic
cgcgccgaggt

SEQ ID NO: 889 - DNA, 33 bp, Artificial Sequence
  Synthetic
tctagccggttttccggctgagacctcggcgcg

SEQ ID NO: 890 - DNA, 27 bp, Artificial Sequence
  Synthetic
tccgcgcgtcccgtatgcaacccttgc

SEQ ID NO: 891 - DNA, 28 bp, Artificial Sequence
  Synthetic
aggccacggacgagtatgcaacccttgc

SEQ ID NO: 892 - DNA, 40 bp, Artificial Sequence
  Synthetic
cttttcacagaactttctgtgcgacgtggttttattccct

SEQ ID NO: 893 - DNA, 63 bp, Artificial Sequence
  Synthetic
tgaggcaagggttgcatacggggaataaaccacgtcgcacagaaagttctgtgaaaaggcttt

SEQ ID NO: 894 - DNA, 63 bp, Artificial Sequence
  Synthetic
tgaggcaagggttgcatactgggaataaaccacgtcgcacagaaagttctgtgaaaaggcttt

SEQ ID NO: 895 - DNA, 34 bp, Artificial Sequence
  Synthetic
tctagccggttttccggctgagaggacgcgcgga

SEQ ID NO: 896 - DNA, 13 bp, Artificial Sequence
  Synthetic
aggccacggacga

SEQ ID NO: 897 - DNA, 35 bp, Artificial Sequence; identical to SEQ ID NO: 879
  Synthetic

SEQ ID NO: 898 - DNA, 38 bp, Artificial Sequence
  Synthetic
cgcgccgaggatatatatatatataagtaggagagggc

SEQ ID NO: 899 - DNA, 38 bp, Artificial Sequence
  Synthetic
aggccacggacgatatatatatataagtaggagagggc

SEQ ID NO: 900 - DNA, 42 bp, Artificial Sequence
  Synthetic
cagtcaaacattaacttggtgtatcgattggtttttgccatt

SEQ ID NO: 901 - DNA, 81 bp, Artificial Sequence
  Synthetic
ggttcgccctctcctacttatatatatatatatatggcaaaaaccaatcgatacaccaagttaatgtttgactgtgtcacg

SEQ ID NO: 902 - DNA, 79 bp, Artificial Sequence
  Synthetic
ggttcgccctctcctacttatatatatatatatggcaaaaaccaatcgatacaccaagttaatgtttgactgtgtcacg

SEQ ID NO: 903 - DNA, 11 bp, Artificial Sequence
  Synthetic
cgcgccgagga

SEQ ID NO: 904-907 - skipped (4 entries)

SEQ ID NO: 908 - DNA, 41 bp, Artificial Sequence
  Synthetic
  misc_feature (21)..(21): n is a or c.
tctttcccttttgacttcaantcagtcatcagaatttcccc

SEQ ID NO: 909 - DNA, 41 bp, Artificial Sequence
  Synthetic
  misc_feature (21)..(21): n is g or a.
cctcgttgtacatcagagacngagcattttacaccttgaag

SEQ ID NO: 910 - DNA, 41 bp, Artificial Sequence
  Synthetic
  misc_feature (21)..(21): n is t or g.
gcatttgggaagggaaaatcnaattaaaagcctaaactaaa

SEQ ID NO: 911 - DNA, 41 bp, Artificial Sequence
  Synthetic
  misc_feature (21)..(21): n is g or t.
agactcggccttttccagatnagcttcagtgtaagagtggg

SEQ ID NO: 912 - DNA, 41 bp, Artificial Sequence
  Synthetic
  misc_feature (21)..(21): n is c or t.
ttaagtaagccatttaccaangctcagaagaaagaacttga

SEQ ID NO: 913 - DNA, 41 bp, Artificial Sequence
  Synthetic
  misc_feature (21)..(21): n is t or c.
tcttgctacaaaccaaaaaangcagcatggtggtggggagg

SEQ ID NO: 914 - DNA, 41 bp, Artificial Sequence
  Synthetic
  misc_feature (21)..(21): n is t or c.
cagacagtaagaagattctanaccatggcctcatatctatt

SEQ ID NO: 915 - DNA, 41 bp, Artificial Sequence
  Synthetic
  misc_feature (21)..(21): n is c or t.
agatttaaaactccaatttanataaaaagttgccataatag

SEQ ID NO: 916 - DNA, 41 bp, Artificial Sequence
  Synthetic
  misc_feature (21)..(21): n is c or t.
tatagaggttcacacacacangccttcattgcgtgtgcatg

SEQ ID NO: 917 - DNA, 21 bp, Artificial Sequence
  Synthetic
tatagaggttcacacacacaa

SEQ ID NO: 918 - DNA, 29 bp, Artificial Sequence
  Synthetic
atgacgtggcagaccgccttcattgcgtg

SEQ ID NO: 919 - DNA, 25 bp, Artificial Sequence
  Synthetic
cgcgccgaggtgccttcattgcgtg

SEQ ID NO: 920 - DNA, 39 bp, Artificial Sequence
  Synthetic
tgcacacgcaatgaaggcgtgtgtgtgtgaacctctata

SEQ ID NO: 921 - DNA, 39 bp, Artificial Sequence
  Synthetic
tgcacacgcaatgaaggcatgtgtgtgtgaacctctata

SEQ ID NO: 922 - DNA, 21 bp, Artificial Sequence
  Synthetic
tctttcccttttgacttcaat

SEQ ID NO: 923 - DNA, 30 bp, Artificial Sequence
  Synthetic
cgcgccgaggatcagtcatcagaatttccc

SEQ ID NO: 924 - DNA, 34 bp, Artificial Sequence
  Synthetic
atgacgtggcagacctcagtcatcagaatttccc

SEQ ID NO: 925 - DNA, 41 bp, Artificial Sequence
  Synthetic
ggggaaattctgatgactgatttgaagtcaaaagggaaaga

SEQ ID NO: 926 - DNA, 41 bp, Artificial Sequence
  Synthetic
ggggaaattctgatgactgagttgaagtcaaaagggaaaga

SEQ ID NO: 927 - DNA, 21 bp, Artificial Sequence
  Synthetic
tttagtttaggcttttaattt

SEQ ID NO: 928 - DNA, 29 bp, Artificial Sequence
  Synthetic
cgcgccgaggagattttcccttcccaaat

SEQ ID NO: 929 - DNA, 32 bp, Artificial Sequence
  Synthetic
atgacgtggcagaccgattttcccttcccaaa

SEQ ID NO: 930 - DNA, 41 bp, Artificial Sequence
  Synthetic
gcatttgggaagggaaaatctaattaaaagcctaaactaaa

SEQ ID NO: 931 - DNA, 41 bp, Artificial Sequence
  Synthetic
gcatttgggaagggaaaatcgaattaaaagcctaaactaaa

SEQ ID NO: 932 - DNA, 21 bp, Artificial Sequence
  Synthetic
cccactcttacactgaagctt

SEQ ID NO: 933 - DNA, 30 bp, Artificial Sequence
  Synthetic
atgacgtggcagaccatctggaaaaggccg

SEQ ID NO: 934 - DNA, 26 bp, Artificial Sequence
  Synthetic
cgcgccgaggaatctggaaaaggccg

SEQ ID NO: 935 - DNA, 40 bp, Artificial Sequence
  Synthetic
gactcggccttttccagatgagcttcagtgtaagagtggg

SEQ ID NO: 936 - DNA, 40 bp, Artificial Sequence
  Synthetic
gactcggccttttccagattagcttcagtgtaagagtggg

SEQ ID NO: 937 - DNA, 21 bp, Artificial Sequence
  Synthetic
ttaagtaagccatttaccaaa

SEQ ID NO: 938 - DNA, 33 bp, Artificial Sequence
  Synthetic
atgacgtggcagaccgctcagaagaaagaactt

SEQ ID NO: 939 - DNA, 30 bp, Artificial Sequence
  Synthetic
cgcgccgaggtgctcagaagaaagaacttg

SEQ ID NO: 940 - DNA, 41 bp, Artificial Sequence
  Synthetic
tcaagttctttcttctgagcgttggtaaatggcttacttaa

SEQ ID NO: 941 - DNA, 41 bp, Artificial Sequence
  Synthetic
tcaagttctttcttctgagcattggtaaatggcttacttaa

SEQ ID NO: 942 - DNA, 21 bp, Artificial Sequence
  Synthetic
cctccccaccaccatgctgct

SEQ ID NO: 943 - DNA, 31 bp, Artificial Sequence
  Synthetic
cgcgccgaggattttttggtttgtagcaaga

SEQ ID NO: 944 - DNA, 35 bp, Artificial Sequence
  Synthetic
atgacgtggcagacgttttttggtttgtagcaaga

SEQ ID NO: 945 - DNA, 41 bp, Artificial Sequence
  Synthetic
tcttgctacaaaccaaaaaatgcagcatggtggtggggagg

SEQ ID NO: 946 - DNA, 41 bp, Artificial Sequence
  Synthetic
tcttgctacaaaccaaaaaacgcagcatggtggtggggagg

SEQ ID NO: 947 - DNA, 21 bp, Artificial Sequence
  Synthetic
cagacagtaagaagattctaa

SEQ ID NO: 948 - DNA, 30 bp, Artificial Sequence
  Synthetic
cgcgccgaggtaccatggcctcatatctat

SEQ ID NO: 949 - DNA, 31 bp, Artificial Sequence
  Synthetic
atgacgtggcagaccaccatggcctcatatc

SEQ ID NO: 950 - DNA, 41 bp, Artificial Sequence
  Synthetic
aatagatatgaggccatggtatagaatcttcttactgtctg

SEQ ID NO: 951 - DNA, 41 bp, Artificial Sequence
  Synthetic
aatagatatgaggccatggtgtagaatcttcttactgtctg

SEQ ID NO: 952 - DNA, 21 bp, Artificial Sequence
  Synthetic
ctattatggcaactttttatt

SEQ ID NO: 953 - DNA, 35 bp, Artificial Sequence
  Synthetic
atgacgtggcagacgtaaattggagttttaaatct

SEQ ID NO: 954 - DNA, 31 bp, Artificial Sequence
  Synthetic
cgcgccgaggataaattggagttttaaatct

SEQ ID NO: 955 - DNA, 41 bp, Artificial Sequence
  Synthetic
agatttaaaactccaatttacataaaaagttgccataatag

SEQ ID NO: 956 - DNA, 41 bp, Artificial Sequence
  Synthetic
agatttaaaactccaatttatataaaaagttgccataatag

SEQ ID NO: 957 - DNA, 40 bp, Artificial Sequence
  Synthetic
acggacgcggagatatatatatatataagtaggagagggc

SEQ ID NO: 958 - DNA, 81 bp, Artificial Sequence; identical to SEQ ID NO: 901
  Synthetic

SEQ ID NO: 959 - DNA, 13 bp, Artificial Sequence
  Synthetic
acggacgcggaga

SEQ ID NO: 960 - DNA, 35 bp, Artificial Sequence; identical to SEQ ID NO: 878
  Synthetic

SEQ ID NO: 961 - DNA, 35 bp, Artificial Sequence; identical to SEQ ID NO: 864
  Synthetic

SEQ ID NO: 962 - DNA, 36 bp, Artificial Sequence
  Synthetic
cgcgccgaggatatatatatataagtaggagagggc

SEQ ID NO: 963 - DNA, 33 bp, Artificial Sequence; identical to SEQ ID NO: 889
  Synthetic

SEQ ID NO: 964 - DNA, 24 bp, Artificial Sequence
  Synthetic
atatatatataagtaggagagggc

SEQ ID NO: 965 - DNA, 42 bp, Artificial Sequence; identical to SEQ ID NO: 900
  Synthetic

SEQ ID NO: 966 - DNA, 77 bp, Artificial Sequence
  Synthetic
ggttcgccctctcctacttatatatatatatggcaaaaaccaatcgatacaccaagttaatgtttgactgtgtcacg

SEQ ID NO: 967 - DNA, 26 bp, Artificial Sequence
  Synthetic
atatatatatataagtaggagagggc

SEQ ID NO: 968 - DNA, 42 bp, Artificial Sequence; identical to SEQ ID NO: 900
  Synthetic

SEQ ID NO: 969 - DNA, 79 bp, Artificial Sequence; identical to SEQ ID NO: 902
  Synthetic

SEQ ID NO: 970 - DNA, 28 bp, Artificial Sequence
  Synthetic
atatatatatatataagtaggagagggc

SEQ ID NO: 971 - DNA, 42 bp, Artificial Sequence; identical to SEQ ID NO: 900
  Synthetic

SEQ ID NO: 972 - DNA, 81 bp, Artificial Sequence; identical to SEQ ID NO: 901
  Synthetic

SEQ ID NO: 973 - DNA, 30 bp, Artificial Sequence
  Synthetic
atatatatatatatataagtaggagagggc

SEQ ID NO: 974 - DNA, 42 bp, Artificial Sequence; identical to SEQ ID NO: 900
  Synthetic

SEQ ID NO: 975 - DNA, 83 bp, Artificial Sequence
  Synthetic
ggttcgccctctcctacttatatatatatatatatatggcaaaaaccaatcgatacaccaagttaatgtttgactgtgtcacg

SEQ ID NO: 976 - DNA, 34 bp, Artificial Sequence
  Synthetic
cgcgccgaggatatatatataagtaggagagggc

SEQ ID NO: 977 - DNA, 77 bp, Artificial Sequence; identical to SEQ ID NO: 966
  Synthetic

SEQ ID NO: 978 - DNA, 42 bp, Artificial Sequence
  Synthetic
acggacgcggagatatatatatatatataagtaggagagggc

SEQ ID NO: 979 - DNA, 83 bp, Artificial Sequence; identical to SEQ ID NO: 975
  Synthetic

SEQ ID NO: 980 - DNA, 28 bp, Artificial Sequence
  Synthetic
acggacgcggagaggaaccctgtgacat

SEQ ID NO: 981 - DNA, 25 bp, Artificial Sequence
  Synthetic
ccatccagggaagagtggcctgttt

SEQ ID NO: 982 - DNA, 47 bp, Artificial Sequence
  Synthetic
tttgaaatgtcacagggttcctaacaggccactcttccctggatggg

SEQ ID NO: 983 - DNA, 35 bp, Artificial Sequence; identical to SEQ ID NO: 878
  Synthetic

SEQ ID NO: 984 - DNA, 26 bp, Artificial Sequence
  Synthetic
cgcgccgaggcgtatgcaacccttgc

SEQ ID NO: 985 - DNA, 11 bp, Artificial Sequence
  Synthetic
cgcgccgaggc

SEQ ID NO: 986 - DNA, 35 bp, Artificial Sequence; identical to SEQ ID NO: 864
  Synthetic

SEQ ID NO: 987 - DNA, 19 bp, Artificial Sequence
  Synthetic
ctgggctgggagcagcctc

SEQ ID NO: 988 - DNA, 23 bp, Artificial Sequence
  Synthetic
cactcgctggcctgtttcatgtc

SEQ ID NO: 989 - DNA, 22 bp, Artificial Sequence
  Synthetic
ctggaatccggtgtcgaagtgg

SEQ ID NO: 990 - DNA, 20 bp, Artificial Sequence
  Synthetic
ctcggcccctgcactgtttc

SEQ ID NO: 991 - DNA, 22 bp, Artificial Sequence
  Synthetic
gaggcaagaaggagtgtcaggg

SEQ ID NO: 992 - DNA, 23 bp, Artificial Sequence
  Synthetic
agtcctgtggtgaggtgacgagg

SEQ ID NO: 993 - DNA, 23 bp, Artificial Sequence
  Synthetic
gccaccatggtgtctttgctttc

SEQ ID NO: 994 - DNA, 22 bp, Artificial Sequence
  Synthetic
accggattccagctgggaaatg

SEQ ID NO: 995 - DNA, 21 bp, Artificial Sequence
  Synthetic
accgggcacctgtactcctca

SEQ ID NO: 996 - DNA, 22 bp, Artificial Sequence
  Synthetic
gcatgagctaaggcacccagac

SEQ ID NO: 997 - DNA, 27 bp, Artificial Sequence
  Synthetic
acggacgcggagttacagcacaggtgc

SEQ ID NO: 998 - DNA, 28 bp, Artificial Sequence
  Synthetic
cgcgccgaggcaggtagtcggtgagatc

SEQ ID NO: 999 - DNA, 22 bp, Artificial Sequence
  Synthetic
cccgcgccacccacactgagcc

SEQ ID NO: 1000 - DNA, 32 bp, Artificial Sequence
  Synthetic
aagagtagccacgctcggtgaggatcttcatt

SEQ ID NO: 1001 - DNA, 22 bp, Artificial Sequence
  Synthetic
gcagtggcagggggcctggtgt

SEQ ID NO: 1002 - DNA, 27 bp, Artificial Sequence
  Synthetic
atgacgtggcagacggtagcgtgcagc

SEQ ID NO: 1003 - DNA, 24 bp, Artificial Sequence
  Synthetic
cgcgccgaggagtagcgtgcagcc

SEQ ID NO: 1004 - DNA, 41 bp, Artificial Sequence
  Synthetic
gctgggctgcacgctacccaccaggccccctgccactgccc

SEQ ID NO: 1005 - DNA, 41 bp, Artificial Sequence
  Synthetic
gctgggctgcacgctactcaccaggccccctgccactgccc

SEQ ID NO: 1006 - DNA, 22 bp, Artificial Sequence
  Synthetic
caggcggcctcctcggtcacct

SEQ ID NO: 1007 - DNA, 24 bp, Artificial Sequence
  Synthetic
cgcgccgaggcactgctccagcga

SEQ ID NO: 1008 - DNA, 27 bp, Artificial Sequence
  Synthetic
atgacgtggcagaccctgctccagcga

SEQ ID NO: 1009 - DNA, 42 bp, Artificial Sequence
  Synthetic
agaagtcgctggagcagtgggtgaccgaggaggccgcctgcc

SEQ ID NO: 1010 - DNA, 41 bp, Artificial Sequence
  Synthetic
agaagtcgctggagcaggggtgaccgaggaggccgcctgcc

SEQ ID NO: 1011 - DNA, 25 bp, Artificial Sequence
  Synthetic
ccttacccgcatctcccacccccat

SEQ ID NO: 1012 - DNA, 24 bp, Artificial Sequence
  Synthetic
cgcgccgaggagacgcccctttcg

SEQ ID NO: 1013 - DNA, 28 bp, Artificial Sequence
  Synthetic
atgacgtggcagacggacgcccctttcg

SEQ ID NO: 1014 - DNA, 44 bp, Artificial Sequence
  Synthetic
ggggcgaaaggggcgtcttgggggtgggagatgcgggtaagggg

SEQ ID NO: 1015 - DNA, 44 bp, Artificial Sequence
  Synthetic
ggggcgaaaggggcgtcctgggggtgggagatgcgggtaagggg

SEQ ID NO: 1016 - DNA, 24 bp, Artificial Sequence
  Synthetic
gctgggctgggtcccaggtcatct

SEQ ID NO: 1017 - DNA, 27 bp, Artificial Sequence
  Synthetic
cgcgccgaggctgtgctcagttagcag

SEQ ID NO: 1018 - DNA, 30 bp, Artificial Sequence
  Synthetic
atgacgtggcagaccgtgctcagttagcag

SEQ ID NO: 1019 - DNA, 47 bp, Artificial Sequence
  Synthetic
atgagctgctaactgagcacaggatgacctgggacccagcccagccc

SEQ ID NO: 1020 - DNA, 46 bp, Artificial Sequence
  Synthetic
atgagctgctaactgagcacggatgacctgggacccagcccagccc

SEQ ID NO: 1021 - DNA, 30 bp, Artificial Sequence
  Synthetic
ggcagagaacaggtcagccaccactatgct

SEQ ID NO: 1022 - DNA, 33 bp, Artificial Sequence
  Synthetic
atgacgtggcagacgcaggttctcatcattgaa

SEQ ID NO: 1023 - DNA, 30 bp, Artificial Sequence
  Synthetic
cgcgccgaggacaggttctcatcattgaag

SEQ ID NO: 1024 - DNA, 55 bp, Artificial Sequence
  Synthetic
gcagcttcaatgatgagaacctgcgcatagtggtggctgacctgttctctgccgg

SEQ ID NO: 1025 - DNA, 55 bp, Artificial Sequence
  Synthetic
gcagcttcaatgatgagaacctgtgcatagtggtggctgacctgttctctgccgg

SEQ ID NO: 1026 - DNA, 31 bp, Artificial Sequence
  Synthetic
gccaccatggtgtctttgctttcctggtgat

SEQ ID NO: 1027 - DNA, 25 bp, Artificial Sequence
  Synthetic
cgcgccgaggccccatccccctatg

SEQ ID NO: 1028 - DNA, 29 bp, Artificial Sequence
  Synthetic
atgacgtggcagacgcccatccccctatg

SEQ ID NO: 1029 - DNA, 51 bp, Artificial Sequence
  Synthetic
agctcatagggggatggggtcaccaggaaagcaaagacaccatggtggctg

SEQ ID NO: 1030 - DNA, 51 bp, Artificial Sequence
  Synthetic
agctcatagggggatgggctcaccaggaaagcaaagacaccatggtggctg

SEQ ID NO: 1031 - DNA, 21 bp, Artificial Sequence
  Synthetic
ccggggctgtccagtgggcat

SEQ ID NO: 1032 - DNA, 23 bp, Artificial Sequence
  Synthetic
cgcgccgaggcagtgggcaccga

SEQ ID NO: 1033 - DNA, 30 bp, Artificial Sequence
  Synthetic
atgacgtggcagacccgagaagctgaagtg

SEQ ID NO: 1034 - DNA, 51 bp, Artificial Sequence
  Synthetic
gcagcacttcagcttctcggtgcccactgtgcccactggacagccccggcc

SEQ ID NO: 1035 - DNA, 42 bp, Artificial Sequence
  Synthetic
gcagcacttcagcttctcggtgcccactggacagccccggcc

SEQ ID NO: 1036 - DNA, 19 bp, Artificial Sequence
  Synthetic
cccgaagcggcgccgcaat

SEQ ID NO: 1037 - DNA, 24 bp, Artificial Sequence
  Synthetic
cgcgccgaggctgcagagggaggg

SEQ ID NO: 1038 - DNA, 28 bp, Artificial Sequence
  Synthetic
atgacgtggcagacgtgcagagggaggg

SEQ ID NO: 1039 - DNA, 38 bp, Artificial Sequence
  Synthetic
ctgaccctccctctgcagttgcggcgccgcttcgggga

SEQ ID NO: 1040 - DNA, 38 bp, Artificial Sequence
  Synthetic
ctgaccctccctctgcacttgcggcgccgcttcgggga

SEQ ID NO: 1041 - DNA, 27 bp, Artificial Sequence
  Synthetic
ggctagaagcactgrtgcccctggcct

SEQ ID NO: 1042 - DNA, 32 bp, Artificial Sequence
  Synthetic
atgacgtggcagacgtgatagtggccatcttc

SEQ ID NO: 1043 - DNA, 28 bp, Artificial Sequence
  Synthetic
cgcgccgaggatgatagtggccatcttc

SEQ ID NO: 1044 - DNA, 50 bp, Artificial Sequence
  Synthetic
gcaggaagatggccactatcacggccaggggcaycagtgcttctagcccc

SEQ ID NO: 1045 - DNA, 50 bp, Artificial Sequence
  Synthetic
gcaggaagatggccactatcatggccaggggcaycagtgcttctagcccc

SEQ ID NO: 1046 - DNA, 30 bp, Artificial Sequence
  Synthetic
agccttttggaagcgtaggaccttgccagt

SEQ ID NO: 1047 - DNA, 28 bp, Artificial Sequence
  Synthetic
atgacgtggcagacccagcgctgggata

SEQ ID NO: 1048 - DNA, 25 bp, Artificial Sequence
  Synthetic
cgcgccgaggacagcgctgggatat

SEQ ID NO: 1049 - DNA, 50 bp, Artificial Sequence
  Synthetic
ctgcatatcccagcgctggctggcaaggtcctacgcttccaaaaggcttt

SEQ ID NO: 1050 - DNA, 50 bp, Artificial Sequence
  Synthetic
ctgcatatcccagcgctgtctggcaaggtcctacgcttccaaaaggcttt

SEQ ID NO: 1051 - DNA, 29 bp, Artificial Sequence
  Synthetic
ctgagctaggtccagcagcctgaggaaga

SEQ ID NO: 1052 - DNA, 29 bp, Artificial Sequence
  Synthetic
atgacgtggcagaccgagggtcgtcgtac

SEQ ID NO: 1053 - DNA, 25 bp, Artificial Sequence
  Synthetic
cgcgccgaggtgagggtcgtcgtac

SEQ ID NO: 1054 - DNA, 48 bp, Artificial Sequence
  Synthetic
tcgagtacgacgaccctcgcttcctcaggctgctggacctagctcagg

SEQ ID NO: 1055 - DNA, 48 bp, Artificial Sequence
  Synthetic
tcgagtacgacgaccctcacttcctcaggctgctggacctagctcagg

SEQ ID NO: 1056 - DNA, 28 bp, Artificial Sequence
  Synthetic
caaggcagcgggcttcctcttgaacact

SEQ ID NO: 1057 - DNA, 31 bp, Artificial Sequence
  Synthetic
atgacgtggcagacggtcctcaatgctcctv

SEQ ID NO: 1058 - DNA, 28 bp, Artificial Sequence
  Synthetic
cgcgccgaggagtcctcaatgctcctcv

SEQ ID NO: 1059 - DNA, 50 bp, Artificial Sequence
  Synthetic
ggaagaggagcattgaggaccgtgttcaagaggaagcccgctgccttgtg

SEQ ID NO: 1060 - DNA, 50 bp, Artificial Sequence
  Synthetic
ggaagaggagcattgaggactgtgttcaagaggaagcccgctgccttgtg

SEQ ID NO: 1061 - DNA, 46 bp, Artificial Sequence
  Synthetic
aattcacaagcagtcacataactaagcttttgtttacattttaccc

SEQ ID NO: 1062 - DNA, 32 bp, Artificial Sequence
  Synthetic
cgcgccgaggttctccattttcatcaggaagv

SEQ ID NO: 1063 - DNA, 35 bp, Artificial Sequence
  Synthetic
atgacgtggcagactctccattttcatcaggaagv

SEQ ID NO: 1064 - DNA, 73 bp, Artificial Sequence
  Synthetic
gattgcttcctgatgaaaatggagaaggtaaaatgtaaacaaaagcttagttatgtgactgcttgtgaatttg

SEQ ID NO: 1065 - DNA, 72 bp, Artificial Sequence
  Synthetic
gattgcttcctgatgaaaatggagaggtaaaatgtaaacaaaagcttagttatgtgactgcttgtgaatttg

SEQ ID NO: 1066 - DNA, 49 bp, Artificial Sequence
  Synthetic
tgattctctctatatagaaaaatctcatgcaatacagatagaaaataat

SEQ ID NO: 1067 - DNA, 39 bp, Artificial Sequence
  Synthetic
atgacgtggcagacgtagagctaataaatgatttcagcv

SEQ ID NO: 1068 - DNA, 35 bp, Artificial Sequence
  Synthetic
cgcgccgaggatagagctaataaatgatttcagcv

SEQ ID NO: 1069 - DNA, 76 bp, Artificial Sequence
  Synthetic
ttatgctgaaatcatttattagctctacttattttctatctgtattgcatgagatttttctatatagagagaatca

SEQ ID NO: 1070 - DNA, 76 bp, Artificial Sequence
  Synthetic
ttatgctgaaatcatttattagctctatttattttctatctgtattgcatgagatttttctatatagagagaatca

SEQ ID NO: 1071 - DNA, 34 bp, Artificial Sequence
  Synthetic
ggaagttctagccagagcaattaggcaagaaatt

SEQ ID NO: 1072 - DNA, 41 bp, Artificial Sequence
  Synthetic
atgacgtggcagacgaaataaaacacattcaaataggaaav

SEQ ID NO: 1073 - DNA, 38 bp, Artificial Sequence
  Synthetic
cgcgccgaggaaaataaaacacattcaaataggaaagv

SEQ ID NO: 1074 - DNA, 66 bp, Artificial Sequence
  Synthetic
tttcctttcctatttgaatgtgttttatttcatttcttgcctaattgctctggctagaacttccaa

SEQ ID NO: 1075 - DNA, 66 bp, Artificial Sequence
  Synthetic
tttcctttcctatttgaatgtgttttattttatttcttgcctaattgctctggctagaacttccaa

SEQ ID NO: 1076 - DNA, 37 bp, Artificial Sequence
  Synthetic
taggattaccatttatccttcaataaggagagtttct

SEQ ID NO: 1077 - DNA, 33 bp, Artificial Sequence
  Synthetic
atgacgtggcagaccagtttccctactccagcv

SEQ ID NO: 1078 - DNA, 29 bp, Artificial Sequence
  Synthetic
cgcgccgaggaagtttccctactccagcv

SEQ ID NO: 1079 - DNA, 58 bp, Artificial Sequence
  Synthetic
gaatgctggagtagggaaactggaaactctccttattgaaggataaatggtaatccta

SEQ ID NO: 1080 - DNA, 58 bp, Artificial Sequence
  Synthetic
gaatgctggagtagggaaacttgaaactctccttattgaaggataaatggtaatccta

SEQ ID NO: 1081 - DNA, 34 bp, Artificial Sequence
  Synthetic
tgaaaagagttgtggaggaaacgtaggcagggat

SEQ ID NO: 1082 - DNA, 32 bp, Artificial Sequence
  Synthetic
atgacgtggcagacgagcctcctttttcagcv

SEQ ID NO: 1083 - DNA, 28 bp, Artificial Sequence
  Synthetic
cgcgccgaggaagcctcctttttcagcv

SEQ ID NO: 1084 - DNA, 56 bp, Artificial Sequence
  Synthetic
tgaggctgaaaaaggaggctctccctgcctacgtttcctccacaactcttttcatc

SEQ ID NO: 1085 - DNA, 56 bp, Artificial Sequence
  Synthetic
tgaggctgaaaaaggaggctttccctgcctacgtttcctccacaactcttttcatc

SEQ ID NO: 1086 - DNA, 28 bp, Artificial Sequence
  Synthetic
ggcaggctggtggggagaaggtcaatga

SEQ ID NO: 1087 - DNA, 28 bp, Artificial Sequence
  Synthetic
cgcgccgaggtatctctggacctcgtgv

SEQ ID NO: 1088 - DNA, 31 bp, Artificial Sequence
  Synthetic
atgacgtggcagaccatctctggacctcgtv

SEQ ID NO: 1089 - DNA, 50 bp, Artificial Sequence
  Synthetic
ggtgcacgaggtccagagatacattgaccttctccccaccagcctgcccc

SEQ ID NO: 1090 - DNA, 50 bp, Artificial Sequence
  Synthetic
ggtgcacgaggtccagagatgcattgaccttctccccaccagcctgcccc

SEQ ID NO: 1091 - DNA, 26 bp, Artificial Sequence
  Synthetic
ggcaggctggtggggagaaggtcaac

SEQ ID NO: 1092 - DNA, 28 bp, Artificial Sequence
  Synthetic
cgcgccgaggtgtatctctggacctcgy

SEQ ID NO: 1093 - DNA, 32 bp, Artificial Sequence
  Synthetic
atgacgtggcagacggtatctctggacctcgy

SEQ ID NO: 1094 - DNA, 48 bp, Artificial Sequence
  Synthetic
tgcacgaggtccagagatacattgaccttctccccaccagcctgcccc

SEQ ID NO: 1095 - DNA, 48 bp, Artificial Sequence
  Synthetic
tgcacgaggtccagagataccttgaccttctccccaccagcctgcccc

SEQ ID NO: 1096 - DNA, 26 bp, Artificial Sequence
  Synthetic
ggcaggctggtggggagaaggtcaac

SEQ ID NO: 1097 - DNA, 27 bp, Artificial Sequence
  Synthetic
cgcgccgaggtgtatctctggacctcg

SEQ ID NO: 1098 - DNA, 31 bp, Artificial Sequence
  Synthetic
atgacgtggcagacggtatctctggacctcg

SEQ ID NO: 1099 - DNA, 48 bp, Artificial Sequence; identical to SEQ ID NO: 1094
  Synthetic

SEQ ID NO: 1100 - DNA, 48 bp, Artificial Sequence; identical to SEQ ID NO: 1095
  Synthetic

SEQ ID NO: 1101 - DNA, 23 bp, Artificial Sequence
  Synthetic
ggggcaggctggtggggagaagt

SEQ ID NO: 1102 - DNA, 34 bp, Artificial Sequence
  Synthetic
atgacgtggcagacgtcaatgtatctctggaccv

SEQ ID NO: 1103 - DNA, 31 bp, Artificial Sequence
  Synthetic
cgcgccgaggctcaatgtatctctggacctv

SEQ ID NO: 1104 - DNA, 48 bp, Artificial Sequence
  Synthetic
cacgaggtccagagatacattgaccttctccccaccagcctgccccat

SEQ ID NO: 1105 - DNA, 48 bp, Artificial Sequence
  Synthetic
cacgaggtccagagatacattgagcttctccccaccagcctgccccat

SEQ ID NO: 1106 - DNA, 39 bp, Artificial Sequence
  Synthetic
taactggcacaaacagaatatgcaataactgttcagtgc

SEQ ID NO: 1107 - DNA, 33 bp, Artificial Sequence
  Synthetic
cgcgccgaggtatcgtagaagagatgaagagav

SEQ ID NO: 1108 - DNA, 37 bp, Artificial Sequence
  Synthetic
atgacgtggcagacaatcgtagaagagatgaagagav

SEQ ID NO: 1109 - DNA, 64 bp, Artificial Sequence
  Synthetic
tctatctcttcatctcttctacgatacactgaacagttattgcatattctgtttgtgccagtta

SEQ ID NO: 1110 - DNA, 64 bp, Artificial Sequence
  Synthetic
tctatctcttcatctcttctacgattcactgaacagttattgcatattctgtttgtgccagtta

SEQ ID NO: 1111 - DNA, 35 bp, Artificial Sequence
  Synthetic
aaatggaggtcaggaataaaaacagctccatgcct

SEQ ID NO: 1112 - DNA, 28 bp, Artificial Sequence
  Synthetic
atgacgtggcagacggccagggcttctv

SEQ ID NO: 1113 - DNA, 25 bp, Artificial Sequence
  Synthetic
cgcgccgaggagccagggcttctcv

SEQ ID NO: 1114 - DNA, 52 bp, Artificial Sequence
  Synthetic
gtgggagaagccctggccggcatggagctgtttttattcctgacctccattt

SEQ ID NO: 1115 - DNA, 52 bp, Artificial Sequence
  Synthetic
gtgggagaagccctggctggcatggagctgtttttattcctgacctccattt

SEQ ID NO: 1116 - DNA, 26 bp, Artificial Sequence
  Synthetic
gccaggccctctcccacacaaatcct

SEQ ID NO: 1117 - DNA, 37 bp, Artificial Sequence
  Synthetic
atgacgtggcagacgttttcctgaaaatagcaaacay

SEQ ID NO: 1118 - DNA, 33 bp, Artificial Sequence
  Synthetic
cgcgccgaggattttcctgaaaatagcaaacay

SEQ ID NO: 1119 - DNA, 53 bp, Artificial Sequence
  Synthetic
tctgtgtttgctattttcaggaaaacggatttgtgtgggagagggcctggccc

SEQ ID NO: 1120 - DNA, 53 bp, Artificial Sequence
  Synthetic
tctgtgtttgctattttcaggaaaatggatttgtgtgggagagggcctggccc

(19) United States
(12) Patent Application Publication
(10) Pub. No.: US 2006/0160074 A1
(43) Pub. Date: Jul. 20, 2006

(54) Pharmacogenetic DME detection assay methods and kits

(75) Inventors: Erin Dorn, Verona, WI (US); Eric Rasmussen, San Diego, CA (US)

Correspondence Address: MEDLEN & CARROLL, LLP, 101 HOWARD STREET, SUITE 350, SAN FRANCISCO, CA, 94105, US

(73) Assignee: Third Wave Technologies, Inc., Madison, WI (US)

(21) Appl. No.: 10/354,953

(22) Filed: Jan. 30, 2003

Related U.S. Application Data
(63) Continuation-in-part of application No. 10/035,833, filed on Dec. 27, 2001, now abandoned.
(60) Provisional application No. 60/353,166, filed on Jan. 31, 2002.
(60) Provisional application No. 60/353,167, filed on Jan. 31, 2002.
(60) Provisional application No. 60/353,444, filed on Jan. 31, 2002.
(60) Provisional application No. 60/353,165, filed on Jan. 31, 2002.
(60) Provisional application No. 60/372,475, filed on Apr. 15, 2002.
(60) Provisional application No. 60/366,984, filed on Mar. 22, 2002.
(60) Provisional application No. 60/424,578, filed on Nov. 7, 2002.
(60) Provisional application No. 60/371,819, filed on Apr. 11, 2002.
(60) Provisional application No. 60/352,940, filed on Jan. 30, 2002.
(60) Provisional application No. 60/356,326, filed on Feb. 13, 2002.

(51) Int. Cl. C12Q 1/68; C07H 21/04

(52) U.S. Cl. 435/6; 536/24.3

(57) ABSTRACT

The present invention relates to methods for detecting polymorphisms in enzymes related to drug metabolizm (Drug Metabolizing Enzymes or DMEs) such as uridine diphosphate glucuronosyl transferase (UGT) gene promoter, cytochrome p450, with a non-amplified oligonucleotide detection assays. The present invention also relates to pharmacogenetic DME detection assay kits.

The present application claims priority to the following applications:

U.S. Provisional Application 60/353,166, filed Jan. 31, 2002;

U.S. Provisional Application 60/353,167, filed Jan. 31, 2002;

U.S. Provisional Application 60/353,444, filed Jan. 31, 2002;

U.S. Provisional Application 60/353,165, filed Jan. 31, 2002;

U.S. Provisional Application 60/372,475, filed Apr. 15, 2002;

U.S. Provisional Application 60/366,984, filed Mar. 22, 2002;

U.S. Provisional Application 60/424,578, filed Nov. 7, 2002;

U.S. application Ser. No. 10/035,833, filed Dec. 27, 2001;

U.S. Provisional Application 60/371, 819, filed Apr. 11, 2002;

U.S. Provisional Application 60/352,940, filed Jan. 30, 2002; and

U.S. Provisional Application 60/356,326, filed Feb. 13, 2002; all of which are herein incorporated by reference.

### FIELD OF THE INVENTION

The present invention relates to methods for detecting polymorphisms in enzymes related to drug metabolizm (Drug Metabolizing Enzymes or DMEs) such as uridine diphosphate glucuronosyl transferase (UGT) gene promoter, cytochrome p450, with a non-amplified oligonucleotide detection assays. The present invention also relates to pharmacogenetic DME detection assay kits.

### BACKGROUND

As the Human Genome Project nears completion and the volume of genetic sequence information available increases, genomics research and subsequent drug design efforts increase as well. There exists a need for systems and methods that allow for the efficient ordering, development, production and sales of detection assays that can be used in genomics research, drug design, and personalized medicine. A number of institutions are actively mining the available genetic sequence information to identify correlations between genes, gene expression and phenotypes (e.g., disease states, metabolic responses, and the like). These analyses include an attempt to characterize the effect of gene mutations and genetic and gene expression heterogeneity in individuals and populations. However, despite the wealth of sequence information available, information on the frequency and clinical relevance of many polymorphisms and other variations has yet to be obtained and validated. For example, the human reference sequences used in current genome sequencing efforts do not represent an exact match for any one person's genome. In the Human Genome Project (HGP), researchers collected blood (female) or sperm (male) samples from a large number of donors. However, only a few samples were processed as DNA resources, and the source names are protected so neither donors nor scientists know whose DNA is being sequenced. The human genome sequence generated by the private genomics company Celera was based on DNA samples collected from five donors who identified themselves as Hispanic, Asian, Caucasian, or African-American. The small number of human samples used to generate the reference sequences does not reflect the genetic diversity among population groups and individuals. Attempts to analyze individuals based on the genome sequence information will often fail. For example, many genetic detection assays are based on the hybridization of probe oligonucleotides to a target region on genomic DNA or mRNA. Probes generated based on the reference sequences will often fail (e.g., fail to hybridize properly, fail to properly characterize the sequence at specific position of the target) because the target sequence for many individuals differs from the reference sequence. Differences may be on an individual-by-individual basis, but many follow regional population patterns (e.g., many correlate highly to race, ethnicity, geographic local, age, environmental exposure, etc.). With the limited utility of information currently available, the art is in need of systems and methods that can optionally be used in one or more production facilities for acquiring, analyzing, storing, and applying large volumes of genetic information with the goal of providing an array of one or more types of detection assay technologies for research and clinical analysis of biological samples. It is an object of the invention to fill these various needs.

### SUMMARY OF THE INVENTION

The present invention relates to methods for detecting polymorphisms in a uridine diphosphate glucuronosyl transferase (UGT) gene promoter with non-amplified oligonucleotide detection assays. The present invention also relates to pharmacogenetic UGT detection assay kits.

In some embodiments, the present invention provides systems for manufacturing and/or selling pharmacogenetic detection assays, comprising: a) a pharmacogenetic detection assay production component for creating a UGT polymorphism-detecting pharmacogenetic oligonucleotide detection assay; b) a pharmacogenetic detection assay quality control component; and c) a label generator, wherein the label generator comprises a device for providing indicia on a package or package insert related to the UGT polymorphism detecting pharmacogenetic detection assay, wherein the indicia is selected from the group consisting of intended use indicia, patient population indicia, proprietary name indicia, established name indicia, quantity indicia, concentration indicia, source indicia, measure of activity indicia, warning indicia, precaution indicia, storage instruction indicia, reconsitution indicia, expiration date indicia, observable indication of alteration indicia, net quantity of contents indicia, number of tests indicia, manufacturer indicia, packer indicia, distributor indicia, lot number indicia, control number indicia, chemical principle indicia, physiological principle indicia, biological principle indicia, mixing instruction indicia, sample preparation indicia, use of instrumentation indicia, calibration indicia, specimen collection indicia, known interfering substances indicia, step by step outline of recommended procedures from reception of specimen to result indicia, indicia indicative for improving performance, indicia indicative for improving accuracy, list of materials indicia, amount indicia, time indicia used to assure accurate results, positive control indicia, negative control indicia, indicia explaining the calculation of an unknown, formula indicia, limitation of procedure indicia, additional testing indicia, range of expected value indicia, specificity indicia, sensitivity indicia, pertinent reference indicia, batch indicia, and date of issuance of last revision of label indicia.

In certain embodiments, the present invention provides methods for detecting TA5 and TA8 UGT repeats in a sample comprising, contacting a sample comprising a target sequence with a non-amplified oligonucleotide detection assay, and determing if the target contains UGT TA5 and/or TA8 repeats. In some embodiments, the non-amplified oligonucleotide detection assay comprises an INVADER assay.

The present invention provides systems, methods, and kits employing nucleic acid detection assays to screen subjects in order to facilitate drug therapy and avoid problems of toxicity or lack of efficacy. In particular, the present invention provides systems, methods, and kits with a nucleic acid detection assay configured to detect polymorphisms in gene sequences associated with Irinotecan safety or efficacy. In this regard, the present invention allows the identification of subjects as suitable or not suitable for treatment with Irinotecan based on the results of employing the detection assay on a sample from the subject.

The invention also provides systems and methods for ordering, manufacturing and selling detection assays, and instrumentation related thereto. The system includes one or more components, such as a computer-based customer order component for ordering at least one of a plurality of oligonucleotide detection assays, and/or related instrumentation; a detection assay production component for creating the oligonucleotide detection assays; a shipping component for shipping said oligonucleotide detection assays and/or related instrumentation; and a billing component for billing a customer for the oligonucleotide detection assays and/or related instrumentation. Optionally, the billing component comprises a payment receipt component for receiving payment for the oligonucleotide detection assays.

The present invention further provides systems, methods, and compositions that provide comprehensive solutions for the manufacturing, use, analysis, and sales of detection assays (e.g., oligonucleotide detection assays). For example, the present invention provides systems and methods for the ordering of detection assay, including electronic ordering (e.g., over public or private electronic communication networks) by general customers, as well as, distributors, collaborators, health care professionals, individuals, and established long-term customers. The present invention also provides systems and methods for detection assay design, including electronic quality assessment methods of detection assay components and design of primers (e.g., amplification primers) and probes. Assay design is made possible for large numbers of diverse assays (of a single type or of multiple types) and for large-scale production thereof, including the design of panels, research products, and clinical products (e.g., in vitro diagnostic products). The present invention also provides systems and methods for detection assay production, including coordinated synthesis, preparation, and quality control of detection assay components, and also detection assay assembly on a variety of presentation platforms, including 96, 384, 1536 well plates, and combinations thereof, slides, and other presentation platforms. Inventory control systems and methods, and design and production management systems and methods, are also provided for complete detection assays, for detection assay components, reagents for the creation of detection assays, and instrumentation used to manufacture detection assays. The present invention also provides systems and methods for selling detection assays, and systems and methods for assisting detection assay users in the collection and analysis of data produced by the use of the detection assays (of a single variety or of multiple varieties). The present invention also provides systems and methods for collecting, analyzing, and storing data, including detection assay design data and data generated by the use of the detection assays. Each of the components of the systems and methods of the present invention may be integrated to provide comprehensive systems and methods for the manufacture and use of detection assays, with exchange of data between various components of the system to optimize utilization of the data generated by the detection assay or detection assay usage. Integration provides, by way of further example, methods to coordinate the movement of genetic information from research applications to in vitro diagnostic applications. Each of the components of the present invention are described in detail below.

In some embodiments, the computer based customer order entry component further comprises a consumer direct web order entry component. Consumers, include by way of example, the purchasing public. The computer based customer order entry component further includes home or work computers, workstations, PDAs or web appliances of members of the public. In other embodiments, the computer-based customer order entry component provides a unidirectional, bi-directional or omni-directional data feed into the detection assay production component, other components of the system and/or portions thereof. In certain embodiments, the data feed affects production cycles of the oligonucleotide detection assays. In particular embodiments, the data feed comprises statistical information associated with or related to one or more oligonucleotide detection assays of a single variety or one or more oligonucleotide detection assays of one or more varieties. In other embodiments, the statistical information is selected from the group consisting of total oligonucleotide detection assays ordered or oligonucleotide detection assay orders received; a histogram; an oligonucleotide detection assay average per consumer; an arithmetic mean; quantity of oligonucleotide detection assays, size of order of oligonucleotide detection assays; format of panel information; a mode; a median; a weighted mean; a harmonic mean; a geometric mean; a logarithmic mean; a root mean square; a root sum square, and combination thereof; a normal distribution curve, the normal distribution curve includes, but is not limited to, a normal distribution curve of number of consumers, number of detection assays, quantity of oligonucleotide detection assays, quantity of oligonucleotide detection assays or a certain type; a spread; a variance; a standard deviation; a skewed distribution; a sampling; a confidence level; and, a regression analysis.

In some embodiments, the present invention provides a system and method for manufacturing and selling detection assays, comprising one or more of the following components: a computer-based customer order component for ordering at least one of a plurality of oligonucleotide detection assays; a detection assay production component for creating the oligonucleotide detection assays of one or more varieties; a shipping component for shipping the oligonucleotide detection assays; and a billing component for billing a customer for the oligonucleotide detection assays. In some embodiments, the billing component comprises a payment receipt component for receiving payment for the oligonucleotide detection assays.

In some embodiments, the computer-based customer order component comprises a client-based computer network, a physician's computer network, and insurance company computer network, a health maintenance organizations computer network, a hospital computer network, a distributor-based computer network, and/or a combination thereof. In some preferred embodiments, the computer-based customer order component comprises a web-based user interface for ordering the oligonucleotide detection assay via single or multiple linked screens or web pages. In some preferred embodiments, the web-based user interface provides a detection assay locator component. For example, in some embodiments, the detection assay locator component comprises a library of detection assay data from which an oligonucleotide detection assay can be selected from a single type of detection assays or from a catalogue of different types of detection assays. In some preferred embodiments, the library of detection assay data comprises single nucleotide polymorphism (“SNP”) data or other data related to the SNP data.

In some embodiments, the detection assay production component comprises a shop floor control system (e.g. comprising an oligonucleotide control system for synthesizing oligonucleotides, and a centralized control network for processing oligonucleotides). In some embodiments, the shop floor control system is configured to direct oligonucleotide detection assay production using a make-to-order routine, a make-to-stock routine, and/or a fulfill-from-stock routine, or other software package. In some embodiments, the shop floor control system comprises a library of detection assay data from which the plurality of detection assays of a single variety or detection assays of more than one variety can be created. It is appreciated that this library of data, the accuracy of which has been checked against a single or plurality of databases of this type of data reduces the error rates associated with detection assay production.

In certain embodiments, the order entry component or the billing component comprises a differential pricing component. The differential pricing component is a set of routines that run on one or more processors of the system described herein. In other embodiments, the differential pricing component is capable of selectably pricing a detection assay or a single variety or a plurality of detection assays of more than one variety based upon a predetermined category of product. In some embodiments, the predetermined category of product is selected from the group consisting of an RUO product, an ASR product, and an IVD product. These routines analyze the product category selection of a consumer or other purchaser to correlate the correct pricing for a detection assay with the category selected by the consumer or the end user. In additional embodiments, the differential pricing component comprises a routine that associates a predetermined price of a detection assay based upon a presentation platform selection. For example, if a consumer selects a 96 well plate as the detection assay presentation platform one price data set is correlated with the transaction. If the consumer selects a combination of different presentation platforms, e.g. 1536 well format, and glass slide format the routines correlate and tabulate the correct price data for the transaction.

In some embodiments, the detection assay production component comprises a synthesis component, a cleave/deprotect component, a purification component, a dilute and fill component, and/or a quality control component. In some embodiments, the synthesis component comprises a plurality of oligonucleotide synthesizers or a single synthesizer capable of a multiplicity of syntheses. The present invention is not limited by the nature of the synthesizers. Synthesizers include, but are not limited to, alone or in combination, MOSS EXPEDITE 16-channel DNA synthesizers (PE Biosystems, Foster City, Calif.), OligoPilot (Amersham Pharmacia,), the 3900 and 3948 48-Channel DNA synthesizers (PE Biosystems, Foster City, Calif.), POLYPLEX (Genemachines), 8909 EXPEDITE, Blue Hedgehog (Metabio), MerMade (BioAutomation, Plano, Tex.), Polygen (Distribio, France), and PrimerStation 960 (Intelligent Bio-Instruments, Cambridge, Mass.). Other synthesizers used herein are those that are capable of simultaneously creating 384 wells and 1536 wells of oligonucleotides. In some embodiments, the detection assay production component comprises an inventory control component. The inventory control component comprises hardware, software, an optional freezer or cooler (walk in style cooler in one variant) with selectable temperature control, and robotics to place and select items of inventory in predetermined locations within the freezer, cooler or cold room.

The present invention is not limited by the nature of the detection assay. In some embodiments, the detection assay comprises an invasive cleavage assay, a TAQMAN assay; a sequencing assay, a polymerase chain reaction assay, a hybridization assay, a hybridization assay employing a probe complementary to a mutation, a microarray assay (e.g. on a solid support), a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay. In some embodiments, the detection assay is configured to detect a sequence selected comprising a polymorphism, a transgene, a splice junction, a mammalian sequence, a prokaryotic sequence, and a plant sequence. It is appreciated that one or more of these detection assays can be produced in one or more production facilities using the systems and methods of the present invention. Moreover, one ore more of these detection assays have data associated or related to each respective detection assay presented via the detection assay locator. By way of further example a particular location on the detection assay locator web page or screen can have listings for several types of detection assay for a single nucleotide polymorphism including pricing information for each respective detection assay. Moreover, it is appreciated that the pricing data located thereon can be variable. For example, where there are three types of detection assay on a page, a routine automatically makes pricing for a favored or predetermined detection assay lower or competitive with one or more other types of detection assays.

In some embodiments, the detection assay production component comprises an oligonucleotide detection assay design component. In some preferred embodiments, the detection assay design component comprises a PCR primer creation component that can optionally be used alone or in combination with the detection assay design component. In some embodiments, the PCR primer creation component is configured to optimize PCR primer concentrations. In some embodiments, the detection assay design component is configured to design a single type of detection assay, a plurality of detections assays of a single variety, or a plurality of detection assays or multiple varieties for detecting the presence of one or more polymorphisms (e.g., single nucleotide polymorphisms), RNA, other sequences and/or combinations thereof. In some embodiments, the detection assay design component is configured to design a panel or array comprising a plurality of oligonucleotide detection assays of a single variety, of multiple varieties, for a single SNP, for multiple SNPS, for a single SNP detected by multiple varieties of detection assays, and for multiple SNPs detected by multiple varieties of detection assays. In some preferred embodiments, the detection assay production component comprises a genotyping component. In some embodiments, the genotyping component is configured to test an oligonucleotide detection assay (of a single type or multiple types) against a plurality of target sequences from different sources.

In some embodiments, the present invention provides detection assay ordering systems, comprising a first processor (including one or more microprocessors) in electronic communication with: a) a computer system or single computer of a customer; b) an electronic detection assay identification catalogue going across one or more genomic landscapes; c) a second processor (including one or more microprocessors) configured to carry out detection assay design; and d) a third processor (including one or more microprocessors) configured to carry out detection assay production. It is appreciated that processors one through three can be a single processor or multiple processors located in one or more locations. Moreover, it is appreciated that archival backup routines and devices provide back up for the data and routines used on one or more devices and components described herein. In some embodiments, the detection assay comprises an invasive cleavage assay or other assay described herein. In other embodiments, the first processor provides a user interface to the computer system of the customer. In particular embodiments, the user interface comprises stacked databases, or linked web pages. In further embodiments, the stacked databases, screens or web pages comprise SNP data or sequence data that includes a SNP. In certain embodiments, the stacked databases or web pages comprise pre-existing detection assay data. In some embodiments, the pre-existing detection assay comprises data of a detection assay that has passed through an in silico process. In particular embodiments, the pre-existing detection assay data comprises data of a detection assay that has passed through a genotyping process.

The present invention provides systems and methods for acquiring and analyzing biological information obtained from the use of one or more types or varieties of detection assays ordered or produced using the systems and methods described herein. For example, the present invention provides systems and methods for the use of genetic information in the generation of assays for detecting the genetic identity of samples, the production of assays, the use of assays for gathering genetic information of individuals and populations, and the storage, analysis, and use of the obtained information.

For example, the present invention provides a method for screening candidate oligonucleotides for use in a detection assay, comprising, providing 1) a candidate oligonucleotide, 2) five or more target nucleic acids (e.g., 6, 7, 8, . . . , 100, . . . ), wherein each of the five or more target nucleic acids is derived from a different subject; and detection assay components that permit detection of the target nucleic acids in the presence of a functional detection oligonucleotide; treating together the five or more target nucleic acids with the candidate oligonucleotide in the presence of the detection assay components; and determining if the candidate oligonucleotide is a functional detection oligonucleotide for use with each of the five or more target nucleic acids. In some embodiments, the target nucleic acids comprise a single nucleotide polymorphism. In some embodiments, the candidate oligonucleotide comprises a hybridization probe. In some preferred embodiments, the candidate oligonucleotide is designed to hybridize to a target sequence of at least one of the target nucleic acids. In some embodiments, the target sequence is identified by or selected by in silico analysis. In certain particular embodiments, the detection assay components comprise detections assay components for performing an INVADER assay. In some embodiments, the method further comprises the step of preparing a kit containing the candidate oligonucleotide if the candidate oligonucleotide is determined to be a functional detection oligonucleotide. In some embodiments, the kit comprises instructions, directing a user of the kit to use the kit with samples from subjects suspected of possessing any of the target nucleic acids from which the candidate oligonucleotide was determined to be a functional detection oligonucleotide.

The present invention also provides a method of gathering and storing genomic data derived from a detection assay, comprising providing a detection assay configured to detect the presence or absence of a nucleic acid sequence in a sample; a first computer system comprising one or more computer processors and a computer memory; a second computer system comprising one or more computer processors and computer memory, wherein the computer memory comprises a genomic information database; and a test sample; treating the test sample with the detection assay to generate test result data; collecting the test result data with the first computer system; and transmitting the test result data from the first computer system to the second computer system under conditions such that the test result data is added to the genomic information database of the second computer system. In some embodiments, the detection assay comprises assays including, but not limited to, hybridization assays, cleavage assays, amplification assays, sequencing assays, and ligation assays. In some preferred embodiments, the detection comprises an INVADER assay, a TAQMAN assay, any other type of assay described herein, and/or combinations thereof. In some embodiments, the nucleic acid sequence comprises a single nucleotide polymorphism or RNA. In some preferred embodiments, the first computer system or computer including a microprocessor comprises one more detectors (e.g., fluorescent detectors, luminescent detectors, optical detectors, and radioactivity detectors). It is appreciated that the instrumentation described herein can also be sold as kit which would include the instrumentation described herein as well as a plurality of pre-ordered or ordered detection assays. In some embodiments, the test sample comprises a genomic DNA or RNA sample or a synthetic DNA or RNA sample. In other embodiments, the test sample comprises an RNA sample, and/or a PCR target/sample. In some embodiments, the test result data comprise information related to a subject from which the test sample was derived. Test result data can be presented to a user via a computer or workstation communicatively linked to any computer or display linked to any of the components described herein. In some embodiments, the first computer system (which is optionally networked) or computer is located in a different geographic location from the second computer system (which is optionally networked in a LAN, MAN, WAN, or combination thereof) or computer. In some embodiments, the transmitting comprises sending the test result data over a communication network on which the various computers are communicatively linked. In some preferred embodiments, the test result data comprises allele frequency information. In other preferred embodiments, the genomic information database comprises database data comprising allele frequency information, genetic location pathway data, metabolic pathway data, and/or combinations thereof.

The present invention further provides a method for searching nucleic acid databases comprising providing a central node comprising a processor, a plurality of sub-nodes in electronic communication with the central node, said sub-nodes comprising sequence database information, and nucleic acid sequence to be searched; providing the nucleic acid sequence to be searched to the central node; and concurrently sending the nucleic acid sequence information to be searched from the central node to the plurality of sub-nodes; and searching the sequence database information with the nucleic acid sequence to be searched to generate search results. In some embodiments, the method further comprises the step of sending the search results from the plurality of sub-nodes to the central node. In preferred embodiments, the latter steps are complete in two seconds or less. In some embodiments, two or more distinct sequence databases are stored on the plurality of sub-nodes. In some embodiments, one of the two or more distinct sequence databases is stored on two or more of the plurality of sub-nodes. In some embodiments, two or more copies of the two or more distinct sequence databases are stored on the plurality of sub-nodes. In some embodiments, each of the plurality of sub-nodes comprises a single sequence database. In some embodiments, the nucleic acid sequence to be searched comprises a single nucleotide polymorphism or RNA. In some preferred embodiments, the sequence and variation in that sequence information comprises one or more databases comprising GoldenPath, GenBank, dbSNP, UniGene, LocusLink, The SNP Consortium, the Japanese SNP, and HGBASE SNP, Ensemble databases.

The present invention also provides a system or method used in one or more components hereof for characterizing a target sequence comprising: screening the target sequence for the presence of repeat sequences and heterologous sequences to generate a masked target sequence; searching a plurality of sequence databases with the masked target sequence to generate search result data; and generating a report comprising the search result data. In some embodiments, the plurality of sequence databases comprises one or more databases including, but not limited to, polymorphism databases, genome databases, linkage databases, and disease association databases (e.g., GoldenPath, GenBank, dbSNP, UniGene, LocusLink, and SNP Consortium databases). In some embodiments, the target sequence comprises a single nucleotide polymorphism. In some preferred embodiments, the report provides a reliability score, said reliability score representing a likelihood of success of detecting the target sequence performance in a detection assay. In some embodiments, the report indicates the presence or absence of the target sequence in one or more of the plurality of sequence databases. In some embodiments, the report indicates a position of the target sequence in a genome. In some embodiments, the report provides polymorphism information related to the target sequence.

The present invention further provides a database (e.g. used in one or more components hereof) comprising allele frequency information, said allele frequency information generated by a method comprising: producing a detection assay for detecting a target sequence; testing five or more target sequences from different subjects with the detection assay to produce assay data; and storing the assay data in a database, wherein the assay data is correlated to at least one characteristic of the subjects. In some embodiments, the target sequence comprises a single nucleotide polymorphism. In some embodiments, the at least one characteristic of the subjects comprises subject age, sex, race or disease state.

The present invention also provides a method for collecting genomic information comprising, providing: a detection assay that detects the presence of a target nucleic acid sequence in a sample, a software application on a computer system of a user, said software application configured to receive detection assay data, a database on a computer system of a service provider, a communications network, and one or more samples comprising nucleic acid; treating the one or more samples with the detection assay to generate assay data; collecting the assay data with the software application; transmitting the assay data from the computer system of the user to the computer system of the service provider using the communications network; and storing the assay data in the database. In some embodiments, the target nucleic acid sequence comprises a single nucleotide polymorphism, wherein the detection assay detects the presence or absence of the single nucleotide polymorphism. The present invention also provides databases generated by such methods. The databases are used in one or more components hereof.

The present invention provides methods, systems, processes, and routines for developing and optimizing nucleic acid detection assays for use in basic research, clinical research, and for the development of clinical detection assays.

In some embodiments, the present invention provides methods comprising; a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises a forward and a reverse primer sequence for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set. It is also appreciated that, in one variant, a customer provided sequence, is automatically augmented upstream and downstream to allow appropriate primer design using the methods and systems described herein.

In other embodiments, the present invention provides methods comprising; a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises a forward and a reverse primer sequence for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In particular embodiments, a method (including computer programs and routines that provide the following functionality) comprising; a) providing target sequence information for at least Y target sequences, wherein each of the target sequences compnses; i) a footprint region, ii) a 5′ region immediately upstream of the footprint region, and iii) a 3′ region immediately downstream of the footprint region, and b) processing the target sequence information such that a primer set is generated, wherein the primer set comprises; i) a forward primer sequence identical to at least a portion of the 5′ region for each of the Y target sequences, and ii) a reverse primer sequence identical to at least a portion of a complementary sequence of the 3′ region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[ 1]-3′ of any of the forward and reverse primers in the primer set.

In other embodiments, the present invention provides methods (including routines that provide the following functionality) comprising a) providing target sequence information for at least Y target sequences, wherein each of the target sequences comprises; i) a footprint region, ii) a 5′ region immediately upstream of the footprint region, and iii) a 3′ region immediately downstream of the footprint region, and b) processing the target sequence information such that a primer set is generated, wherein the primer set comprises; i) a forward primer sequence identical to at least a portion of the 5′ region for each of the Y target sequences, and ii) a reverse primer sequence identical to at least a portion of a complementary sequence of the 3′ region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In particular embodiments, the present invention provides methods (and routines providing the following functionality) comprising a) providing target sequence information for at least Y target sequences, wherein each of the target sequences comprises a single nucleotide polymorphism, b)determining where on each of the target sequences one or more assay probes would hybridize in order to detect the single nucleotide polymorphism such that a footprint region is located on each of the target sequences, and c) processing the target sequence information such that a primer set is generated, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In some embodiments, the present invention provides methods (and routines providing the following functionality) comprising a) providing target sequence information for at least Y target sequences, wherein each of the target sequences comprises a single nucleotide polymorphism, b) determining where on each of the target sequences one or more assay probes would hybridize in order to detect the single nucleotide polymorphism such that a footprint region is located on each of the target sequences, and c) processing the target sequence information such that a primer set is generated, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide T or G, and N[2]-N[1]-3′ of each of the forward and reverse primers is not complementary to N[2]-N[1]-3′ of any of the forward and reverse primers in the primer set.

In certain embodiments, the primer set is configured for performing a multiplex PCR reaction that amplifies at least Y amplicons, wherein each of the amplicons is defined by the position of the forward and reverse primers. In other embodiments, the primer set is generated as digital or printed sequence information. In some embodiments, the primer set is generated as physical primer oligonucleotides. Using the methods, routines and components herein is it possible to generate 100-plex and greater PCR primer reactions.

In certain embodiments, N[3]-N[2]-N[1]-3′ of each of the forward and reverse primers is not complementary to N[3]-N[2]-N[1]-3′ of any of the forward and reverse primers in the primer set. In other embodiments, the processing comprises initially selecting N[1] for each of the forward primers as the most 3′ A or C in the 5′ region. In certain embodiments, the processing comprises initially selecting N[1] for each of the forward primers as the most 3′ G or T in the 5′ region. In some embodiments, the processing comprises initially selecting N[1] for each of the forward primers as the most 3′ A or C in the 5′ region, and wherein the processing further comprises changing the N[1] to the next most 3′ A or C in the 5′ region for the forward primer sequences that fail the requirement that each of the forward primer's N[2]-N[1]-3′ is not complementary to N[2]-N[1]-3′ of any of the forward and reverse primers in the primer set.

In other embodiments, the processing (preferably electronic) comprises initially selecting N[1] for each of the reverse primers as the most 3′ A or C in the complement of the 3′ region. In some embodiments, the processing comprises initially selecting N[1] for each of the reverse primers as the most 3′ G or T in the complement of the 3′ region. In further embodiments the processing comprises initially selecting N[1] for each of the reverse primers as the most 3′ A or C in the 3′ region, and wherein the processing further comprises changing the N[1] to the next most 3′ A or C in the 3′ region for the reverse primer sequences that fail the requirement that each of the reverse primer's N[2]-N[1]-3′ is not complementary to N[2]-N[1]-3′ of any of the forward and reverse primers in the primer set.

In particular embodiments, the footprint region comprises a single nucleotide polymorphism. In some embodiments, the footprint comprises a mutation. In some embodiments, the footprint region for each of the target sequences comprises a portion of the target sequence that hybridizes to one or more assay probes configured to detect the single nucleotide polymorphism. In certain embodiments, the footprint is this region where the probes hybridize. In other embodiments, the footprint further includes additional nucleotides on either end.

In some embodiments, the processing (electronic in one variant of the invention) further comprises selecting N[5]-N[4]-N[3]-N[2]-N[1]-3′ for each of the forward and reverse primers such that less than 80 percent homology with a assay component sequence is present. In preferred embodiments, the assay component is a FRET probe sequence. In certain embodiments, the target sequence is about 300-500 base pairs in length, or about 200-600 base pair in length. In certain embodiments, Y is an integer between 2 and 500, or between 2-10,000.

In certain embodiments, the processing (electronic in one variant of the invention) comprises selecting x for each of the forward and reverse primers'such that each of the forward and reverse primers has a melting temperature with respect to the target sequence of approximately 50 degrees Celsius (e.g. 50 degrees, Celsius, or at least 50 degrees Celsius, and no more than 55 degrees Celsius). In preferred embodiments, the melting temperature of a primer (when hybridized to the target sequence) is at least 50 degrees Celsius, but at least 10 degrees different than a selected detection assay's optimal reaction temperature.

In some embodiments, the forward and reverse primer pair optimized concentrations are determined for the primer set. In other embodiments, the processing is automated. In further embodiments, the processing is automated with a processor.

In other embodiments, the present invention provides a kit comprising the primer set generated by the methods of the present invention, and at least one other component (e.g. cleavage agent, polymerase, INVADER oligonucleotide, or other detection assay or detection assay component in another variant of the invention). In certain embodiments, the present invention provides compositions comprising the primers and primer sets generated by the methods of the present invention.

In particular embodiments, the present invention provides methods (and routines utilizing methodology) comprising; a) providing; i) a user interface configured to receive sequence data, ii) a computer system having stored therein a multiplex PCR primer software application, and b) transmitting the sequence data from the user interface to the computer system, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, and c) processing the target sequence information with the multiplex PCR primer pair software application to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set.

In some embodiments, the present invention provides methods (and routines used in the methodology) comprising; a) providing; i) a user interface configured to receive sequence data, ii) a computer system having stored therein a multiplex PCR primer software application, and b) transmitting the sequence data from the user interface to the computer system, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, and c) processing the target sequence information with the multiplex PCR primer pair software application to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set.

In certain embodiments, the present invention provides systems comprising; a) a computer system (and routines used in the methodology) configured to receive data from a user interface, wherein the user interface is configured to receive sequence data, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, b) a multiplex PCR primer pair software application operably linked to the user interface, wherein the multiplex PCR primer software application is configured to process the target sequence information to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide A or C, and N[2]-N[1]-3′ of each of the forward and reverse primers is not complementary to N[2]-N[1]-3′ of any of the forward and reverse primers in the primer set, and c) a computer system having stored therein the multiplex PCR primer pair software application, wherein the computer system comprises computer memory and a computer processor.

In other embodiments, the present invention provides systems comprising; a) a computer system or computer configured to receive data from a user interface, wherein the user interface is configured to receive sequence data, wherein the sequence data comprises target sequence information for at least Y target sequences, wherein each of the target sequences comprises; i) a footprint region, ii) a 5′ region immediately upstream of the footprint region, and iii) a 3′ region immediately downstream of the footprint region, b) a multiplex PCR primer pair software application operably linked to the user interface, wherein the multiplex PCR primer software application is configured to process the target sequence information to generate a primer set, wherein the primer set comprises; i) a forward primer sequence identical to at least a portion of the target sequence immediately 5′ of the footprint region for each of the Y target sequences, and ii) a reverse primer sequence identical to at least a portion of a complementary sequence of the target sequence immediately 3′ of the footprint region for each of the at least Y target sequences, wherein each of the forward and reverse primer sequences comprises a nucleic acid sequence represented by 5′-N[x]-N[x-1]- . . . -N[4]-N[3]-N[2]-N[1]-3′, wherein N represents a nucleotide base, x is at least 6, N[1] is nucleotide G or T, and N[2]-N[1]-3′ of each of the forward and reverse primers is not complementary to N[2]-N[1]-3′ of any of the forward and reverse primers in the primer set, and c) a computer system having stored therein the multiplex PCR primer pair software application, wherein the computer system comprises computer memory and a computer processor. In certain embodiments, the computer system is configured to return the primer set to the user interface.

The present invention relates to novel methods of producing oligonucleotides. In particular, the present invention provides an efficient, safe, and automated process for the production of large quantities of oligonucleotides.

In some embodiments, the present invention provides high-throughput oligonucleotide production systems comprising: an oligonucleotide synthesizer component, wherein the oligonucleotide synthesizer component comprises at least 100 oligonucleotide synthesizers. In particular embodiments, the system further comprises at least one oligonucleotide processing component. In certain embodiments, the system further comprises a centralized control network operably linked to the oligonucleotide synthesizer component.

In particular embodiments, the present invention provides methods for the high through-put production of oligonucleotides comprising; a) providing an oligonucleotide synthesizer component; and b) generating a high through-put quantity of oligonucleotides with the oligonucleotide synthesizer component, wherein the high through-put quantity comprises at least 1 per hour (e.g. at least 1, 10, 100, 1000, etc, per hour).

In some embodiments, the present invention provides methods for the production of an oligonucleotide comprising: a) providing; i) a first computer memory device comprising oligonucleotide specification information, and ii) an oligonucleotide synthesizer component, wherein the oligonucleotide synthesizer component comprises a) at least 100 oligonucleotide synthesizers (in another variant the number of synthesizers can be in the range of about 20 to about 1000 synthesizers depending on the number of syntheses each synthesizer is capable of executing), and b) a second computer memory device; and b) conveying the oligonucleotide specification information from the first computer memory device to the second computer memory device under conditions such that the oligonucleotide synthesizer component generates at least one oligonucleotide (e.g. at least 1, 10, 100, 1000, etc). In another variant of the invention where high throughput synthesizers are used it is possible to substitute fewer synthesizers but still accomplish a desired level of syntheses.

In certain embodiments, the present invention provides oligonucleotide production systems comprising: a) an oligonucleotide production component configured for divergent production of a set of oligonucleotides, wherein the set of oligonucleotides comprises first and second corresponding oligonucleotides, and wherein the oligonucleotide production component comprises first and second oligonucleotide manufacturing components; and b) a centralized control network operably linked to the oligonucleotide production component, wherein the centralized control network is configured for controlling the divergent production of the set of oligonucleotides.

In other embodiments, the present invention provides methods for the divergent production of oligonucleotides comprising; a) providing an oligonucleotide production component comprising an oligonucleotide synthesizer component and at least one oligonucleotide processing component; and b) employing the oligonucleotide production component for divergent production of a set of oligonucleotides, wherein the set of oligonucleotides comprises first and second corresponding oligonucleotides.

In some embodiments, the present invention provides high-throughput oligonucleotide purification systems comprising a plurality of HPLC devices operably connected to a single sample injector. In other embodiments, the system further comprises a centralized control network.

In particular embodiments, the present invention provides methods for the high-throughput purification of oligonucleotides comprising: a) providing; i) an oligonucleotide purification component comprising a plurality of HPLC devices operably connected to a single sample injector, and ii) an oligonucleotide sample comprising full-length oligonucleotides and truncated oligonucleotides; and b) processing the sample with the oligonucleotide purification component under conditions such that at least a portion of the truncated oligonucleotides are removed from the oligonucleotide sample.

In some embodiments, the present invention provides high-throughput oligonucleotide production systems comprising; a) an oligonucleotide production component comprising first and second oligonucleotide manufacturing components; and b) a sample rack configured for use in the first and second oligonucleotide manufacturing components without modification. In particular embodiments, the system further comprises a central reagent supply network.

In certain embodiments, the present invention provides methods for high-throughput processing of oligonucleotide samples, comprising: a) providing; i) an oligonucleotide production component comprising first and second manufacturing components, and ii) a sample rack integrated with the first manufacturing component, wherein the sample rack is configured for use in the first and second oligonucleotide manufacturing components without modification, and wherein the sample rack comprises a plurality of oligonucleotide samples; and b) processing at least a portion of the plurality of oligonucleotide samples with the first manufacturing component, c) transferring the sample rack from the first manufacturing component to the second manufacturing component; and d) processing at least a portion of the oligonucleotide samples with the second manufacturing component.

In particular embodiments, the present invention provides high-throughput oligonucleotide dry-down systems comprising a centrifugal evaporator configured for processing at least 1 aqueous oligonucleotide sample in one hour or less. In particular embodiments, the system is configured for processing at least 5 oligonucleotide samples per hour (e.g. 5, 10, 15, 20, 25, 30, 35, 40, 45, 50, or more than 50). In different embodiments, the present invention provides high-throughput oligonucleotide dry down systems comprising a centrifugal evaporator configured for processing a plurality of oligonucleotide samples in one hour or less, wherein the plurality of oligonucleotide samples comprises at least 1 liter of water (e.g. 1, 5, 10, 15, 35 or 50 liters of water).

In some embodiments, the present invention provides methods for the high-throughput dry-down of oligonucleotides comprising: a) providing; i) an oligonucleotide dry-down component comprising a centrifugal evaporator, and ii) a plurality of oligonucleotide samples comprising at least 10 aqueous oligonucleotide samples; and b) processing the plurality of oligonucleotide samples with the oligonucleotide dry-down component, wherein the processing renders each of the aqueous oligonucleotide samples substantially water-free in one hour or less.

In certain embodiments, the present invention provides methods for the high-throughput dry-down of oligonucleotides comprising: a) providing; i) an oligonucleotide dry-down component comprising a centrifugal evaporator, and ii) a plurality of aqueous oligonucleotide samples, wherein the plurality of oligonucleotide samples comprises at least one liter of water, and b) processing the plurality of oligonucleotide samples with the oligonucleotide dry-down component, wherein the processing renders the plurality of aqueous oligonucleotide samples substantially water-free in one hour or less.

In some embodiments, the present invention provides high-throughput oligonucleotide de-salting systems comprising an oligonucleotide de-salting component configured for processing at least 150 oligonucleotide samples per half hour. In particular embodiments, the oligonucleotide de-salting component comprises a robotic oligonucleotide sample handling device, and a sample rack.

In other embodiments, the present invention provides methods for the high-throughput de-salting of oligonucleotides comprising: a) providing; i) an oligonucleotide de-salting component comprising a robotic oligonucleotide sample handling device, and ii) a plurality of oligonucleotide samples comprising at:least 150 oligonucleotide samples; and b) processing the plurality of oligonucleotide samples with the oligonucleotide de-salting component, wherein the processing renders each of the oligonucleotide samples substantially salt-free in a half-hour or less.

In other embodiments, the present invention provides high-throughput oligonucleotide dilute and fill systems comprising an oligonucleotide dilute and fill component, wherein the oligonucleotide dilute and fill component comprises an automated liquid processing device operably linked to a spectrophotometer.

In some embodiments, the present invention provides methods method for the high-throughput dilute and fill of oligonucleotide samples comprising: a) providing; i) an oligonucleotide dilute and fill component comprising an automated liquid processing device operably linked to a spectrophotometer, and ii) a plurality of oligonucleotide samples; and b) processing the plurality of oligonucleotide, samples with the oligonucleotide dilute and fill component, wherein the processing normalizes each of the oligonucleotide samples. It is appreciated that normalization of concentration is an important aspect of the invention with respect to the production of detection assays. In one variant, oligonucleotide production samples have their concentrations normalized. This normalization can be accomplished via the utilization of known extinction coefficient methods and knowledge of the sequence from production information.

The present invention also provides a nucleic acid synthesis reagent delivery system comprising: one or more reagent containers containing nucleic acid synthesis reagent; a branched delivery component attached to said one or more reagent containers such that the nucleic acid synthesis reagent can pass from said reagent containers to said branched delivery component, wherein the branched delivery component comprises a plurality of branches; and a plurality of delivery lines, the plurality of delivery lines attached on one end to a branch of the branched delivery component and attached on a second end to a nucleic acid synthesizer. The present invention is not limited by the number branches or delivery lines. In some embodiments, the plurality of branches comprises ten or more branches. In some embodiments, the plurality of delivery lines comprises ten or more delivery lines. In some embodiments, the branched delivery component comprises a sight glass. In some preferred embodiments, the sight glass comprises a purge valve. In yet other embodiments, the one or more of the plurality of delivery lines comprises a shut-off valve.

The present invention further provides a waste disposal system comprising: a waste tank comprising a waste input channel configured to receive liquid waste product and a waste output channel configured to remove liquid waste when the waste tank is purged; and a pressurized gas line attached to the waste tank, the pressurized gas line configured to deliver gas into the waste tank when the waste tank is to be purged, wherein the gas line is configured to deliver a gas that allows purging of the waste tank. In some embodiments, the pressurized gas line is attached to an argon gas source. In preferred embodiments, the gas is delivered at a low pressure (e.g., 3-10 pounds per square inch). In some embodiments, the waste input channel is attached to a waste line, wherein the waste line is attached to a plurality of nucleic acid synthesizers (e.g., 20 or more nucleic acid synthesizers). In some preferred embodiments, the waste tank comprises a sight glass. In other preferred embodiments, the system further comprises an automated purge component, said automated purge component capable of detecting waste levels in the waste tank and purging the waste tank when the waste levels are at or above a threshold level (e.g., a pre-selected threshold level).

The present invention also provides a method for purifying nucleic acids comprising providing: an nucleic acid purification column, a buffer, and a nucleic acid mixture; contacting the nucleic acid mixture with the nucleic acid purification column; and adding the buffer to the nucleic acid purification column, wherein a nucleic acid molecule having between 23-39 nucleotides is eluted from the nucleic acid purification column in less than forty minutes, and in one variant of the invention can be accomplished in less than about 25 minutes. In some embodiments, the nucleic acid purification column is contained in an HPLC apparatus.

The present invention further provides a method for deprotecting nucleic acid molecules comprising providing: a multiwell plate configured to hold a plurality of protected nucleic acid molecules and a plurality of different protected nucleic acid molecules; placing the nucleic acid molecules into the multiwell plates; and treating the plate under conditions that resulted in the deprotection of the nucleic acid molecules. In some embodiments, the multiwell plate comprises a 96-well plate.

The present invention relates to nucleic acid synthesizers and methods of using and modifying nucleic acid synthesizers. For example, the present invention provides highly efficient, reliable, and safe synthesizers that find use, for example, in high throughput and automated nucleic acid synthesis, as well as methods of modifying pre-existing synthesizers to improve efficiency, reliability, and safety. The present invention also relates to synthesizer arrays for efficient, safe, and automated processes for the production of large quantities of oligonucleotides.

In some embodiments, the present invention provides systems comprising a synthesis and purge component, the synthesis and purge component comprising a cartridge and a drain plate, wherein the cartridge is configured to hold one or more nucleic acid synthesis columns and wherein the cartridge is separated from the drain plate by a drain plate gasket. In certain embodiments, the cartridge is configured to hold a plurality of nucleic acid synthesis columns. In particular embodiments, the cartridge is configured to hold 12 or more nucleic acid synthesis columns. In other embodiments, the cartridge is configured to hold 48 or more nucleic acid synthesis columns. In additional embodiments, the cartridge is configured to hold exactly 48 nucleic acid synthesis columns.

In some embodiments, the assembly comprising the cartridge, the drain plate and the drain plate gasket is configured to provide a substantially airtight seal between the assembly and the outside of each nucleic acid synthesis column. In one embodiment, the airtight seal between the assembly and each column is provided by an O-ring. In a preferred embodiment, each O-ring is positioned between the cartridge and the exterior surface of a column. In yet another variant, any material that provides a compressible interface can be used in the invention.

In certain embodiments, the drain plate gasket provides a substantially airtight seal between the cartridge and the drain plate. In other embodiments, the drain plate gasket provides an airtight seal between the cartridge and the drain plate. In some embodiments, the drain plate gasket comprises one or more alignment markers configured to allow aligned attachment of said cartridge to said drain plate. In additional embodiments, the drain plate gasket comprises one or more alignment markers configured to allow aligned attachment of the drain plate gasket to the cartridge. In other embodiments, the drain plate gasket comprises one or more alignment markers configured to allow aligned attachment of the gasket to the drain plate. In certain embodiments, the drain plate gasket comprises at least one drain cut-out. In other embodiments, the drain plate gasket comprises at least four drain cut-outs. In still other embodiments, the drain plate gasket comprises one drain cut out for every synthesis column in the cartridge. In yet other embodiments, the cut outs in the drain plate gasket for each synthesis column are configured to provide an airtight seal between the outside of each nucleic acid synthesis column and the assembly comprising the cartridge, the drain plate, and the drain plate gasket.

In some embodiments, the present invention provides systems comprising a synthesis and purge component, the synthesis and purge component comprising a cartridge and a drain plate, wherein the cartridge is configured to hold one or more nucleic acid synthesis columns and wherein the cartridge is separated from the drain plate by a drain plate gasket. In some embodiments, the drain plate comprises at least one drain (e.g. 1, 2, 3, 4, 5, 10, . . . 20, . . . ). In other embodiments, the system further comprises a waste tube, the waste tube comprising input and output ends, wherein the input end is configured to receive waste materials from the drain. In particular embodiments, the waste tube comprises an inner diameter of at least 0.187 inches (preferably at least 0.25 inches). In some embodiments, the waste tube and the drain are configured such that, when the drain is contacted with the waste tube for waste removal, the waste tube encloses at least a portion of the drain (See, e.g., FIG. 40). In particular embodiments, the drain forms a sealed contact point with an interior portion of the waste tube when the drain is enclosed in the waste tube. In still other embodiments, the drain further comprises a drain sealing ring. In certain embodiments, the system further comprises a waste valve wherein the waste valve is configured to receive waste from the output end of the waste tube. In particular embodiments, the waste valve comprises an interior diameter of at least 0.187 inches (preferably at least 0.25 inches). In some embodiments, the waste valve provides a straight-through path for the waste (e.g. as opposed to an angled path). Straight-through paths can be accomplished, for example, by the use of a gate or ball valve.

In some embodiments, the system further comprises a plurality of dispense lines, the dispense line configured for delivering at least one reagent to a synthesis column in the cartridge. In certain embodiments, the dispense lines comprise an interior diameter of at least 0.25 mm. In particular embodiments, the system further comprises an alignment detector. In particular embodiments, the alignment detector is configured to detect the alignment of a waste tube and a drain. In other embodiments, the alignment detector is configured to detect the alignment of a dispense line and a receiving hole of the cartridge. In some embodiments, the alignment detector is configured to detect a tilt alignment of the synthesis and purge component.

In some embodiments, the system of the present invention further comprises a motor attached to the synthesis and purge component and configured to rotate the synthesis and purge component. In particular embodiments, the motor is attached to the synthesis and purge component by a motor connector. In further embodiments, the system further comprises a bottom chamber seal positioned between the motor connector and the synthesis and purge component. In certain embodiments, the system of the present invention comprises two drain. In preferred embodiments; the two drain are located on opposite sides of the drain plate.

In some embodiments of the systems of the present invention, the synthesis and purge component is contained in a chamber. In certain embodiments, a chamber bowl and a top cover (when in place) combine to form a chamber (e.g. which may be pressurized, for example, with inert gas). One example is depicted in FIG. 34 where chamber bowl 18 and top cover 30 combine to form an exemplary chamber. In some embodiments, the chamber comprises a bottom surface (e.g. bottom of a chamber bowl, see, e.g. FIG. 41) comprising the top portion of two waste tubes (which may, for example, extend downward from bottom of the chamber). In preferred embodiments, the waste tubes are positioned symmetrically on the bottom surface of the chamber (see, e.g., FIG. 41).

In particular embodiments, the systems of the present invention further comprise a chamber drain having open and closed positions, the chamber drain configured to allow gas emissions (or liquid waste) to pass out of the chamber when in the open position.

In some embodiments, the systems of the present invention further comprise a reagent dispensing station, wherein the reagent dispensing station is configured to house one or more reagent reservoirs, such that reagents in reagent reservoirs can be delivered to the cartridge. In certain embodiments, the reagent dispensing station comprises one or more ventilation tubes (e.g., connected to one or more ventilation valves of the reagent dispensing station) configured to remove gaseous emissions from the reagent dispensing station. In certain embodiments, the reagent dispensing station provides an enclosure. In preferred embodiments, the enclosure comprises a viewing window to allow visual inspection of the reagent reservoirs without opening the enclosure. In preferred embodiments, one reagent dispensing station is configured to serve multiple synthesizers.

In particular embodiments, the systems of the present invention are capable of maintaining a gas pressure in the chamber sufficient to purge synthesis columns prior to addition of reagents to the synthesis columns.

In some embodiments, the nucleic acid synthesis systems of the present invention comprise a cartridge in a chamber, the cartridge comprising a plurality of synthesis columns, wherein the synthesis columns contain packing material that provides a resistance against pressurized gas contained in the chamber, the resistance being sufficient to maintain a pressure in the chamber that is capable of purging synthesis columns prior to addition of reagents to the synthesis columns. In certain embodiments, one or more of the plurality of synthesis columns does not undergo a synthesis reaction. In particular embodiments, two or more different lengths of oligonucleotides are synthesized in the plurality of synthesis columns. In other embodiments, the packing material comprises a frit. In some embodiments, the frit is a bottom frit. In other embodiments, the frit is a top frit. In preferred embodiments, the packing material comprises a top frit, solid support, and a bottom frit. In particularly preferred embodiments, the solid support is polystyrene. In some embodiments, the packing material comprises a synthesis matrix.

In some embodiments, the present invention provides nucleic acid synthesis systems comprising a synthesis and purge component in a pressurized chamber, the synthesis and purge component comprising a plurality of synthesis columns, wherein the synthesis columns contain packing material sufficient to maintain pressure in the chamber during a purging operation to purge liquid reagent from the plurality of synthesis columns when at least one of the plurality of synthesis columns does not contain liquid reagent. In certain embodiments, more than one of the plurality of synthesis columns (e.g. 2, 3, 5, 10) do not contain liquid reagent (and the remaining synthesis columns do contain liquid reagent).

In certain embodiments, the present invention provides nucleic acid synthesis systems comprising: a) a synthesis and purge component, the synthesis and purge component comprising a cartridge and a drain plate separated by a drain plate gasket, wherein the cartridge is configured to hold twelve or more nucleic acid synthesis columns; b) a drain positioned in the drain plate; c) a chamber comprising an inner surface, the chamber housing the synthesis and purge component and the drain; d) a waste tube, the waste tube comprising input and output ends, wherein the input end is configured to receive waste materials from the drain, wherein the waste tube comprises an inner diameter of at least 0.187 inches; e) a waste valve configured to receive waste from the output end of the waste tube, wherein the waste valve comprises in interior diameter of at least 0.187 inches; f) a reagent dispensing station, wherein the reagent dispensing station is configured to house one or more reagent reservoirs; g) a plurality of dispense lines, the dispense lines configured for delivering reagents from the reagent reservoirs to a synthesis column in the cartridge, wherein the dispense lines comprise an interior diameter of at least 0.25 mm) a rotating motor attached to the synthesis and purge component by a motor connector and configured to rotate the synthesis and purge component; and i) a gas line configured to release gas into the chamber to create a gas pressure in the chamber greater than a gas pressure in the waste tube. In certain embodiments, the system is capable of maintaining gas pressure in the chamber at a sufficient level to purge the synthesis columns prior to addition of reagents to the synthesis columns.

In some embodiments, the synthesizer further comprises providing energy, such as heat, to the synthesis columns. Heating of the synthesis column finds use, for example, in decreasing the coupling time during a nucleic acid synthesis. It can also broaden the range of the chemical protocols that can be used in high throughput synthesis, e.g. by improving the efficiency of less efficient chemistries, such as the phosphate triester method of oligonucleotide synthesis. In other embodiments, the synthesizer further comprises a mixing component, such as an agitator, configured to agitate the synthesis columns (e.g., to mix reaction components, and to facilitate mass exchange between the reaction medium and the solid support).

In some embodiments, the present invention provides methods for synthesizing nucleic acids comprising: a) providing: i) a nucleic acid synthesizer comprising a synthesis and purge component, the synthesis and purge component comprising a cartridge and a drain plate, wherein the cartridge holds a plurality of nucleic acid synthesis columns and wherein the cartridge is separated by a drain plate gasket from the drain plate, and ii) nucleic acid synthesis reagents; and b) introducing a portion of the nucleic acid synthesis reagents into at least one of the nucleic acid synthesis columns to provide a first synthesis reaction; c) purging the nucleic acid synthesis columns by creating a pressure differential across the nucleic acid synthesis columns; and d) introducing a second portion of the nucleic acid synthesis reagents into at least one of the nucleic acid synthesis columns to provide a second synthesis reaction. In particular embodiments, the drain plate gasket provides a substantially airtight seal between the cartridge and the drain plate. In other embodiments, the drain plate gasket provides an airtight seal between the cartridge and the drain plate.

The present invention further provides a cartridge for use in an open nucleic acid synthesis system, said cartridge comprising a plurality of receiving holes configured to hold nucleic acid synthesis columns, wherein the cartridge is further configured to receive one or more O-rings, wherein the presence of the one or more O-rings provides a seal between the nucleic acid synthesis columns and the plurality of receiving holes (i.e., the O-ring contacts an interior wall of the receiving hole and an exterior wall of the synthesis column to form a seal). In some embodiments, the cartridge is provided as part of a nucleic acid synthesis system: The present invention is not limited by the nature of the O-ring. For example, in some embodiments, the cartridge is associated with a gasket, wherein the gasket provides the O-rings (e.g., through one or more holes in the gaskets, such that when the gasket is associated with the cartridge [e.g., affixed to an outer surface of the cartridge] a seal is formed between the a receiving hole of the cartridge and a synthesis column within the receiving hole [see e.g., FIG. 46C]). In other embodiments, the O-ring is provided in a groove within the receiving hole. For example, in some embodiments, the groove is located at the top surface of the receiving hole. In such embodiments, the plurality of receiving holes comprise an upper portion and a lower portion, wherein the lower portion comprises a first diameter and the upper portion comprises a second diameter that is larger than the first diameter (see e.g., FIG. 46A). In other embodiments, the groove is located within an interior portion of the receiving hole. In such embodiments, the plurality of receiving holes comprise an upper portion with a first diameter, a middle portion with a second diameter, and a lower portion with a third diameter, wherein the second diameter is larger than the first diameter and larger than the third diameter (the first and third diameters may be the same as each other or different). When an O-ring is placed in the groove, the O-ring contains an internal diameter less than the first diameter and less than the third diameter, such that it can contact a synthesis column placed within the receiving hole (see e.g., FIG. 46B).

In-some embodiments, the cartridge comprises a rotary cartridge. In some preferred embodiments, O-rings are provided in the cartridge. In some preferred embodiments, the O-ring is configured to form a substantially airtight or pressure-tight seal between the receiving hole and the nucleic acid synthesis column, when said nucleic acid synthesis column is present.

The present invention further provides a nucleic acid synthesis system comprising a synthesis and purge component in a pressurizable chamber, said synthesis and purge component comprising a cartridge, wherein the cartridge in configured to hold a plurality of nucleic acid synthesis columns, and wherein said cartridge is further configured to provides seals between said cartridge and each of said plurality of nucleic acid synthesis columns so as to maintain pressure in said chamber during a purging operation to purge liquid reagent from said plurality of synthesis columns. In some embodiments, each of the seals between the cartridge and the plurality of nucleic acid synthesis columns is provided by an O-ring.

In some embodiments, the present invention provides a nucleic acid synthesizer comprising a plurality of synthesis columns and an energy input component that imparts energy to said plurality of synthesis columns to increase nucleic acid synthesis reaction rate in said plurality of synthesis columns. In some embodiments, said energy input component comprises a heating component. In preferred embodiments, said heating component provides substantially uniform heat. In some embodiments, said energy input component provides heated reagent solutions to said plurality of synthesis columns. In other embodiments, said energy input component comprises a heating coil. In yet other embodiments, said energy input component comprises a heat blanket. In yet other embodiments, said heating component comprises a resistance heater, a Peltier device, a magnetic induction device or a microwave device. In still other embodiments, said energy input component comprises a heated room. In further embodiments, said energy input component provides energy in the electromagnetic spectrum. In yet other embodiments, said energy input component comprises an oscillating member. In some embodiments, said energy input component provides a periodic energy input, and in other embodiments, said energy input component provides a constant energy input.

In some preferred embodiments, said energy input heats said plurality of synthesis columns in the range of about 20 to about 60 degrees Celsius.

In some embodiments, the present invention provides a nucleic acid synthesizer comprising a fail-safe reagent delivery component configured to deliver one or more reagent solutions to said plurality of synthesis columns. In some embodiments, the fail-safe reagent delivery component comprises a plurality of reagent tanks. In preferred embodiments, said plurality of reagent tanks comprise one or more tanks selected from the group consisting of acetonitrile tanks, phosphoramidite tanks, argon gas tanks, oxidizer tanks, tetrazole tanks, and capping solution tanks. In some particularly preferred embodiments, said reagent tanks comprise a plurality of large volume containers, each said large volume container comprising at least one of said reagent solutions. In some embodiments, the present invention provides high-throughput oligonucleotide production systems comprising: an oligonucleotide synthesizer array, wherein the oligonucleotide synthesizer array comprises at least 5 oligonucleotide synthesizers. In preferred embodiments, the oligonucleotide synthesizer array comprises at least 10 or at least 100 oligonucleotide synthesizers. In certain embodiments, the system further comprises a centralized control network operably linked to the oligonucleotide synthesizer component.

In particular embodiments, the present invention provides methods for the high through-put production of oligonucleotides comprising; a) providing an oligonucleotide synthesizer array; and b) generating a high through-put quantity of oligonucleotides with the oligonucleotide synthesizer array, wherein the high through-put quantity comprises at least 1 per hour (e.g. at least 1, 10, 100, 1000, etc, per hour).

The present invention provides a production facility comprising an array of synthesizers. In some embodiments, the production facility of the present invention comprises a fail-safe reagent delivery system. In other embodiments, the production facility of the present invention comprises a centralized waste collection system. In yet other embodiments, the production facility of the present invention comprises a centralized control system. In preferred embodiments, the production facility of the present invention comprises a fail-safe reagent delivery system, a centralized waste collection system and a centralized control system.

In some embodiments, the present invention provides an automated production process. In some embodiments, the automated production process includes an oligonucleotide synthesizer component and an oligonucleotide-processing component.

The present invention also provides integrated systems that link nucleic acid synthesizers to other nucleic acid production components. For example, the present invention provides a system comprising a nucleic acid synthesizer and a cleavage and deprotect component. In some embodiments, the synthesizer is configured for parallel synthesis of nucleic acid molecules in three or more synthesis columns. In some embodiments, the system further comprises sample tracking software configured to associate sample identification tags (e.g., electronic identification numbers, barcodes) with samples that are processed by the nucleic acid synthesizer and the cleavage and deprotect component. In some preferred embodiments, the sample tracking software is further configured to receive synthesis request information from a user, prior to sample processing by the nucleic acid synthesizer. In some embodiments, the system further comprises a robotic component configured to transfer columns from the nucleic acid synthesizer to the cleavage and deprotect component. In other preferred embodiments, the robotic component is further configured to transfer the columns from the cleavage and deprotect component to a purification component and/or to additional production components described herein.

The present invention also provides control systems for operating one or more components of the systems of the present invention. For example, the present invention provides a system comprising a processor, wherein the processor is configured to operate a nucleic acid synthesizer for parallel synthesis of three or more nucleic acid molecules. The present invention further provides a system comprising a processor, wherein said processor is configured to operate a nucleic synthesizer and a cleavage and deprotect component. In some embodiments, the system further comprises a computer memory, wherein the computer memory comprises nucleic acid sample order information (e.g., information obtained from a user specifying the identity of a polymer to be synthesized and/or specifying one or more characteristics of the polymer such as sequence information). In some embodiments, the computer memory further comprises allele frequency information and/or disease association information.

In some embodiments, the present invention provides oligonucleotide synthesizers comprising a reaction chamber and a lid, wherein in an open position, the lid provides a substantially enclosed ventilated workspace. In certain embodiments, the present invention provides methods of protecting an operator of an oligonucleotide synthesizer comprising channeling ambient air away from an operator toward an interior space of the synthesizer (e.g. down through the top surface, or up through the top cover). In other embodiments, the present invention provides apparatuses comprising, in combination, an oligonucleotide synthesizer and a venting hood. In some embodiments, the apparatuses are for production of oligonucleotides, wherein the apparatus comprises a venting component configured to draw air away from a reaction chamber of the apparatus. In certain embodiments, the present invention provides systems comprises a plurality of oligonucleotide apparatuses (e.g. e.g. at least 100 synthesizers).

In particular embodiments, the present invention provides a polymer synthesizer comprising a ventilated workspace. In some embodiments, certain embodiments, the polymer synthesizer is a nucleic acid synthesizer. In certain embodiments, the synthesizer comprises a top enclosure, wherein the top enclosure comprises a top plate with a ventilation opening, wherein the top enclosure is configured for attachment to a top cover of a synthesizer to form a primarily enclosed space over the top cover. In other embodiments, the synthesizer comprises a base, wherein the base comprises a primarily enclosed space and a ventilation opening.

In certain embodiments, the top plate is configured for attachment to a ventilation tube such that air in the primarily enclosed space may be drawn through the ventilation opening into the ventilation tube. In other embodiments, the top plate further comprises an outer window, and wherein the ventilation opening is formed in the outer window. In certain embodiments, the top enclosure further comprises at least four sides (e.g. 4 sides, 5 sides, etc.). In certain embodiments, the top cover further comprises a ventilation slot.

In certain embodiments, the present invention provides polymer synthesizer (e.g. nucleic acid synthesizer) comprising; a) a top cover with a ventilation slot, and b) a top enclosure, wherein the top enclosure comprises a top plate with a ventilation opening, and wherein the top enclosure is attached to the top cover to form a primarily enclosed space above the top cover.

In certain embodiments, the present invention provides a lid enclosure comprising; a) a top cover with a ventilation slot, and b) a top enclosure, wherein the top enclosure comprises a top plate with a ventilation opening, and wherein the top enclosure is attached to the top cover to form a primarily enclosed space over the top cover. In certain embodiments, the top plate is configured for attachment to a ventilation tube. In particular embodiments, the top plate is configured for attachment to a ventilation tube such that air in the primarily enclosed space may be drawn through the ventilation opening into the ventilation tube. In other embodiments, the top cover is configured to attach to a top surface of a nucleic acid synthesizer with a chamber bowl.

In some embodiments, the ventilation slot is configured such that air in the chamber bowl may drawn in through the ventilation slot and into the primarily enclosed space. In other embodiments, the top plate further comprises an outer window, and wherein the ventilation opening is formed in the outer window. In certain embodiments, the top enclosure further comprises at least four sides.

In certain embodiments, the present invention provides a polymer synthesizer (e.g., nucleic acid synthesizer) comprising; a) a top surface of a nucleic acid synthesizer, b) a lid enclosure comprising; i) a top plate with a ventilation opening, and ii) a top cover with a ventilation slot; and wherein the lid enclosure is attached to the top surface. In some embodiments, the lid enclosure is attached to the top surface by at least one hinge such that the lid enclosure may be raised and lowered. In certain embodiments, the present invention provides systems comprises a plurality of the polymer synthesizers (e.g., at least 100 synthesizers).

In some embodiments, the present invention provides side panels configured to extend between at least one side of a top cover (or lid enclosure) and a top surface of a nucleic acid synthesizer such that a barrier to air is created on at least one side of the synthesizer when the top cover is extended upward from the top surface. In other embodiments, the present invention provides a panel (e.g. front panel or side panel) configured to extend at least part way between at least one side of a top cover (or lid enclosure) and a top surface of a nucleic acid synthesizer such that at least a partial barrier to air is created on at least one side of the synthesizer when the top cover is extended upward such that it is not in contact with the top surface. In other embodiments, the present invention provides polymer synthesizers (e.g. nucleic acid synthesizers) summary comprising; a) a top surface of a nucleic acid synthesizer, b) a lid enclosure comprising; i) a top plate with a ventilation opening, ii) a top cover with a ventilation slot; and iii) at least one top enclosure side; and c) a panel; wherein the lid enclosure is attached to the top surface by at least one hinge such that the lid enclosure may be raised and lowered, and wherein the panel is configured to extend (at least part way) between the at least one top enclosure side and the top surface such that at least a partial barrier to air is created when the lid enclosure is extended upward from the top surface. In certain embodiments, the present invention provides systems comprising a plurality of the polymer synthesizers (e.g., at least 100 synthesizers).

In particular embodiments, the present invention provides systems comprising; a) a ventilation tube, and b) a lid enclosure comprising; a) a top cover with a ventilation slot, and b) a top enclosure comprising a top plate with a ventilation opening, wherein the top enclosure is attached to the top cover to form a primarily enclosed space over the top cover. In some embodiments, the systems further comprise a vacuum source (e.g. centralized vacuum system).

In certain embodiments, the top plate is configured for attachment to the ventilation tube. In other embodiments, the ventilation tube is configured for attachment to the vacuum source. In particular embodiments, the system further comprises a synthesis and purge component, the synthesis and purge component comprising a cartridge and a drain plate separated by a drain plate gasket, wherein the cartridge is configured to hold a plurality of nucleic acid synthesis columns. In some embodiments, the systems further comprise a plurality of dispense lines, wherein the plurality of dispense lines are located in the primarily enclosed space.

In certain embodiments, the systems further comprise at least one side panel, wherein the at least one side panel is configured to extend between at least one side of the lid enclosure and a top surface of a nucleic acid synthesizer (e.g., such that a barrier to air is created on at least one side of the synthesizer when the top cover is extended upward from the top surface).

In some embodiments, the present invention provides systems comprising; a) a nucleic acid synthesizer comprising; i) a top surface, and ii) a top cover comprising a ventilation slot, wherein the top cover is attached to the top surface by at least one hinge such that the top surface may be raised and lowered; and b) a panel configured to extend at least part way between at least one side of the top cover and the top surface such that at least a partial barrier to air is created on at least one side of the nucleic acid synthesizer when the top cover is extended upward. In other embodiments, the panel is configured to fully extend between the at least one side of the top cover and the top surface such that a complete barrier to air is created on at least one side of the nucleic acid synthesizer when the top cover is extended upward. In some embodiments, the panel comprises a side panel or a front panel.

In certain embodiments, the system further comprises a top enclosure, wherein the top enclosure comprises a top plate with a ventilation opening, and wherein the top enclosure is attached to the top cover to form a primarily enclosed space over the top cover. In other embodiments, the system further comprises a ventilation tube. In particular embodiments, the system further comprises a vacuum source. In other embodiments, the vacuum source comprises a centralized vacuum system. In particular embodiments, the top plate is configured for attachment to the ventilation tube. In certain embodiments, the ventilation tube is configured for attachment to the vacuum source.

In some embodiments, the present invention provides methods comprising forming a ventilation opening in a top plate of a top enclosure such that the top plate is configured for attachment to a ventilation tube. In certain embodiments, the present invention provides methods comprising; a) providing; i) a top enclosure comprising a top plate, and ii) a ventilation tube; and b) forming a ventilation opening in the top plate, and c) attaching the ventilation tube to the top plate such that the ventilation tube forms a seal around the ventilation opening. In further embodiments, the methods further comprise step d) attaching a least one panel to the top enclosure.

In other embodiments, the present invention provides methods comprising; a) providing; i) a top cover of a nucleic acid synthesizer comprising a ventilation slot, wherein the top cover is configured to be attached to a top surface of a nucleic acid synthesizer such that the top surface may be raised and lowered; and ii) a top enclosure, wherein the top enclosure comprises a top plate with a ventilation opening, and b) attaching the top enclosure to the top cover such that a primarily enclosed space is formed over the top cover. In other embodiments, the methods further comprise the step of attaching at least one panel to the top enclosure (or the top cover), wherein the at least one panel extends at least part way between at least one side of the top cover (or the top cover) and the top surface such that at least a partial barrier to air is created on at least one side of the synthesizer when the top cover is extended upward such that it is not in contact with the top surface.

In particular embodiments, the present invention provides methods comprising; a) providing; i) a nucleic acid synthesizer comprising; i) a top cover with a ventilation slot, and ii) a top enclosure, wherein the top enclosure comprises a top plate with a ventilation opening, wherein the top enclosure is attached to the top cover to form a primarily enclosed space above the top cover, and wherein the top plate is attached to a ventilation tube such that the ventilation tube forms a seal around the ventilation opening, and ii) a vacuum source attached to the ventilation tube, and b) activating the vacuum source such that air is drawn into the ventilation slot, through the primarily open space, and out through the ventilation opening into the ventilation tube.

In some embodiments, the present invention provides kits comprising; a) a top enclosure comprising a top plate with a ventilation opening, wherein the top enclosure is configured for attachment to a top cover of a synthesizer to form a primarily enclosed space over the top cover, and b) a printed material component, wherein the printed material component comprises written instruction for installing the top enclosure onto the top cover.

In other embodiments, the present invention provides kits comprising; a) a panel configured to extend at least part way between at least one side of a top cover (or lid enclosure) and a top surface of a nucleic acid synthesizer such that at least a partial barrier to air is created on at least one side of the synthesizer when the top cover is extended upward such that it is not in contact with the top surface, and b) a printed material component, wherein the printed material component comprises written instructions for installing the panel onto a top cover (or lid enclosure).

The present invention relates to polymer synthesizers and methods of using polymer synthesizers. For example, the present invention provides highly efficient, reliable, and safe synthesizers that find use, for example, in high throughput and automated nucleic acid synthesis. The present invention also relates to synthesizer arrays for efficient, safe, and automated processes for the production of large quantities of oligonucleotides.

For example, the present invention provides a system comprising a closed system solid phase synthesizer configured for parallel synthesis (e.g., simultaneous side-by-side synthesis) of three or more polymers (e.g., 3, 4, 5, 6, 7, . . . , 10, . . . , 48, . . . , 96, . . . ). The present invention is not limited by the nature of the polymer. Polymers include, but are not limited to, nucleic acids and polypeptides. In some preferred embodiments, the nucleic acid polymers comprise DNA. In some particularly preferred embodiments, the DNA comprises an oligonucleotide.

The synthesizers of the present invention allow parallel synthesis of multiple polymers. Each of the synthesized polymers may be identical to one another (e.g., in composition, sequence, length, etc.) or may be different than one another (e.g., in composition, sequence, length, etc.). Thus, the synthesizers of the present invention may be configured to simultaneously produce three or more distinct polymers (e.g., oligonucleotides).

Because the synthesizers of the present invention allow parallel processing of polymers, large numbers of polymers may be produced in a single synthesizer in a short period of time. For example, the synthesizer may be configured to produce 100 or more polymers per day. In some embodiments, the synthesizer may be configured to produce 1000-2000 or more polymers per day. For example, synthesizers may be configured to produce 2000 or more oligonucleotide per day (e.g., oligonucleotides containing 20-40 or more bases). In some preferred embodiments, the produced polymers (e.g., 2000 or more produced polymers) are produced at a 1 μM synthesis scale. In some embodiments, the produced polymers are produced on a micro-scale, e.g., less than 5 nmole synthesis scale. In some preferred embodiments, micro-scale synthesis is performed on a 0.1 to 1 nmole synthesis scale.

The present invention also provides a solid phase synthesizer comprising: a reaction support comprising three or more (e.g., 3, 4, 5, 6, 7, . . . , 10, . . . , 48, . . . , 96, . . . ) reaction chambers (e.g., chambers that are isolated from one another, such that fluid does not pass from one chamber to another during synthesis); and a plurality of reagent dispensers configured to simultaneously form closed fluidic connections with each of the reaction chambers, wherein the reagent dispensers are each configured to deliver all reagents necessary for a polymer synthesis reaction. In some embodiments, the reaction chambers comprise synthesis columns. For example, the reaction support provides a fixed surface to support three or more synthesis columns. In some embodiments, the synthesis columns comprise nucleic acid synthesis columns (e.g., columns designed for use with EXPEDITE nucleic acid synthesizers [Applied Biosystems, Foster City, Calif.], 3900 High-Throughput Columns for use with the 3900 DNA Synthesizer [Applied Biosystems], DNA synthesis columns from Biosearch Technologies, Novato, Calif.). In preferred embodiments, the reaction support is configured to contain and form a tight seal around multiple, different synthesis columns (e.g., of different sizes or from different manufacturers), so as to allow any number of commercially available columns to be used with the synthesizer.

In some embodiments, the reagent dispensers are fluidicly connected to a plurality of reagent tanks (e.g., through tubing). In preferred embodiments, reagent dispensers are constructed from any substantially inert materials including, but not limited to, stainless steel, glass, Teflon, and titanium. Tanks include, but are not limited to, acetonitrile tanks, phosphoramidite tanks, argon gas tanks, oxidizer tanks, tetrazole tanks, and capping solution tanks. In some embodiments, the tanks are contained within the synthesizer. In other embodiments, the tanks are contained on an outer surface of the synthesizer. In some preferred embodiments, tanks are provided separately from the synthesizer (e.g., in a different room, such as an explosion-proof room). For example, in some embodiments, the present invention provides large volume synthesis facilities containing multiple synthesizers, wherein two or more of the synthesizer are serviced by the same reagent tanks. In some such embodiments, “large volume containers” are used as reagent tanks. Individual large volume reagent tanks contain from about 200 liters to about 2500 liters of acetonitrile, from about 200 liters to about 2500 liters of deblocking solution; from about 2 liters to about 200 liters of amidite; from about 20 liters to about 200 liters of activator (e.g., tetrazol); from about 20 liters to about 200 liters of capping reagents; or from about 20 liters to about 200 liters of oxidizer. Alternatively, a plurality of tanks containing a combined capacity as indicated above may be used. In some embodiments, the large volume reagent tanks are connected to a plurality of synthesizers through a large volume reagent delivery system, which allows large volumes of reagents to be delivered simultaneously to each of the synthesizers

Various useful reagents and coupling chemistries are described in U.S. Pat. No. 5,472,672 to Bennan, and U.S. Pat. No. 5,368,823 to McGraw et al. (both of which are herein incorporated by reference in their entireties). In addition to phosphoramidite chemistries, phosphate and phosphite triester methods, and H-phosphonate methods of oligonucleotide synthesis are contemplated.

In some embodiments, the reaction support comprises a fixed reaction support (e.g., a reaction support that does not move during operation). In some embodiments, the reaction support comprises a plurality of waste channels. In preferred embodiments, the waste channels in closed fluidic contact with each of the reaction chambers (See e.g., FIG. 53).

In some embodiments, the synthesizer further comprises providing energy, such as heat to the reaction chambers. Heating of the reaction chamber finds use, for example, in decreasing the coupling time during a nucleic acid synthesis. It can also broaden the range of the chemical protocols that can be used in high throughput synthesis, e.g. by improving the efficiency of less efficient chemistries, such as the phosphate triester method of oligonucleotide synthesis. In other embodiments, the synthesizer further comprises a mixing component, such as an agitator, configured to agitate the reaction chambers (e.g., to mix reaction components, and to facilitate mass exchange between the reaction medium and the solid support).

The present invention further provides a solid phase synthesizer comprising: a fixed reaction support comprising three or more reaction chambers; and a plurality of reagent dispensers configured to simultaneously form closed fluidic connections with each of said reaction chambers.

The present invention also provides integrated systems that link nucleic acid synthesizers to other nucleic acid production components. For example, the present invention provides a system comprising a closed system nucleic acid synthesizer and a cleavage and deprotect component. In some embodiments, the synthesizer is configured for parallel synthesis of nucleic acid molecules at three or more reaction sites. In some preferred embodiments, the system further comprises a reaction support comprising three or more reaction chambers, wherein the reaction support is configured for operation with both the nucleic acid synthesizer and the cleavage and deprotect component. In some embodiments, the system further comprises sample tracking software configured to associate sample identification tags (e.g., electronic identification numbers, barcodes) with samples that are processed by the nucleic acid synthesizer and the cleavage and deprotect component. In some preferred embodiments, the sample tracking software is further configured to receive synthesis request information from a user, prior to sample processing by the nucleic acid synthesizer. In some embodiments, the system further comprises a robotic component configured to transfer the reaction support from the nucleic acid synthesizer to the cleavage and deprotect component. In other preferred embodiments, the robotic component is further configured to transfer the reaction support from the cleavage and deprotect component to a purification component and/or to additional production components described herein.

The present invention also provides control systems for operating one or more components of the systems of the present invention. For example, the present invention provides a system comprising a processor, wherein the processor is configured to operate a close system nucleic acid synthesizer for parallel synthesis of three or more nucleic acid molecules. The present invention further provides a system comprising a processor, wherein said processor is configured to operate a nucleic synthesizer and a cleavage and deprotect component. In some embodiments, the system further comprises a computer memory, wherein the computer memory comprises nucleic acid sample order information (e.g., information obtained from a user specifying the identity of a polymer to be synthesized and/or specifying one or more characteristics of the polymer such as sequence information). In some embodiments, the computer memory further comprises allele frequency information and/or disease association information.

In some embodiments, the present invention relates to detecting mutations in pooled nucleic acid samples. In particular, the present invention relates to compositions and methods for detecting mutations or measuring allele frequencies in pooled nucleic acid samples employing the INVADER detection assay or other detection assays described herein. In some embodiments, the present invention provides methods for detecting an allele frequency of a polymorphism, comprising: a) providing; i) a pooled sample, wherein the pooled sample comprises target nucleic acid sequences from at least 10 individuals (or at least 50, or at least 100, or at least 250, or at least 500, or at least 1000 individuals, etc.); and ii) INVADER detection reagents (e.g. primary probes, INVADER oligonucleotides, FRET cassettes, a structure specific enzyme, etc.) configured to detect the presence or absence of a polymorphism; and b) contacting the pooled sample with the INVADER detection reagents to generate a detectable signal; and c) measuring the detectable signal, thereby determining a number of the target nucleic acid sequences that contain the polymorphism (e.g. a quantitative number of molecules, or the allele frequency for the polymorphism in a population, is determined). In some embodiments, signals from two or more alleles for a particular target nucleic acid locus are measured and the numbers are compared. In preferred embodiments, the measurements for two or more different alleles of a particular target nucleic acid locus are measured in a single reaction. In other embodiments, measurements from one or more alleles of a particular target nucleic acid locus are compared to measurements from one or more reference target nucleic acid loci. In preferred embodiments, measurements from one or more alleles of a particular target nucleic acid locus are compared to measurements from one or more reference target nucleic acid loci in the same reaction mixture. Further methods allow a single individual's particular allele frequency (i.e., frequency of the mutation among multiple copies of the sequence within an individual) or quantitative number of molecules found to possess the polymorphism (e.g. determined by an INVADER assay) to be compared to the population allele frequency (or expected number), such that it is determined if the single individual is susceptible to a disease, how far a disease has progressed (e.g. diseases such as cancer that may be diagnosed by identifying loss of heterozygosity), etc. In some embodiments, the individuals are from the same racial or ethnic class (e.g. European, African, Asian, Mexican, etc).

In particular embodiments, the present invention provides methods for detecting a rare mutation comprising; a) providing; i) a sample from a single subject, wherein the sample comprises at least 10,000 target nucleic acid sequences (e.g. from 10,000 cells, or at least 20,000 target nucleic acid sequences, or at least 100,000 target nucleic acid sequences), ii) a detection assay (e.g. the INVADER assay) capable of detecting a mutation in a population of target nucleic acid sequence that is present at an allele frequency of 1:1000 or less compared to wild type alleles; and b) assaying the sample with the detection assay under conditions such that the presence or absence of a rare mutation (e.g. one present at an allele frequency of 1 :100, or 1:500, or 1:1000 or less compared to the wild type) is detected. In some embodiments, the target nucleic acid sequences are genomic (e.g. not polymerase chain reaction, or PCR, amplified, but directly from a cell). In other embodiments, the target nucleic acid sequences are amplified (e.g., by PCR).

In some embodiments, the present invention provides methods for detecting a rare mutation comprising; a) providing: i) a sample from a single subject, wherein the sample comprises at least 10,000 target nucleic acid sequences, ii) a detection assay capable of detecting a mutation in a population of target nucleic acid sequence that is present at an allele frequency of 1:1000 or less compared to wild type alleles; and b) assaying the sample with the detection assay under conditions such that an allele frequency in the sample of a rare mutation is determined. In some embodiments, the subject's allele frequency is compared statistically to a known reference allele frequency (e.g. determined by the methods of the present invention or other methods), such that a diagnosis may be made (e.g. extent of disease, likelihood of having the disease, or passing it on to offspring, etc).

The present invention also provides methods for determining the number of molecules of one or more polymorphisms present in a sample by employing, for example, the INVADER assay (e.g. polymorphisms such as SNPs that are associated with disease). This assay may be used to determine the number of a particular polymorphism in a first sample, and then determining if there is a statisticall significant difference between that number and the number of the same polymorphism in a second sample. Preferably, one sample represents the number of the polymorphism expected to occur in a sample obtained from a healthy individual, or from a healthy population if pooled samples are used. A statistically significant difference between the number of a polymorphism expected to be at a single-base locus in a healthy individual and the number determined to be in a sample obtained from a patient is clinically indicative.

The present invention relates to detection assay panels comprising an array of different detection assays. The detection assays include assays for detecting mutations in nucleic acid molecules and for detecting gene expression levels. Assays find use, for example, in the identification of the genetic basis of phenotypes, including medically relevant phenotypes and in the development of diagnostic products, including clinical diagnostic products. The present invention also provides systems and methods for data storage, including data libraries and computer storage media comprising detection assay data.

For example, the present invention provides a panel comprising an array, wherein the array comprises a plurality of different assays (e.g., greater than about 50 different assays). In some preferred embodiments, the assays are substantially similar to at least one assay shown in FIG. 96, and in U.S. application Ser. No. 10/035,833 filed Dec. 27, 2001 and which is expressly incorporated by reference in its entirity. In some embodiments, the arrays comprise greater than about 100 different assays (e.g., 100, 101, 102, . . . , 130, . . . , 500, . . . , 1000, . . . , 10,000, . . . , 30,000, . . . ). In some preferred embodiments, the assays comprise biplex assays. In other preferred embodiments, the assays comprise multiplex assays. In some embodiments, the array is a microarray. In some preferred embodiments, the assays are provided on a solid surface. For example, in some embodiments, the assays are provided on a microtiter plate.

In some preferred embodiments, the assays comprise nucleic acid detection assay. For example, in some embodiments, the assays detect polymorphisms (e.g., single-nucleotide polymorphisms in nucleic acids), including direct detection of genomic DNA (e.g., human genomic DNA).

The present invention also provides methods for using panels. For example, the present invention provides a method comprising: a) providing: i) a panel comprising an array, said array comprising a plurality of different assays (e.g., detection assays) and ii) a sample; and b) exposing the sample to the panel under conditions such that at least one of the assays detects the presence of a target nucleic acid in the sample. Any of the panels or detection assays described herein may be used in the method. The present invention also provides system and methods for developing clinical products based on information obtained from the use of the panels. Systems and methods are also provided for collecting, storing, and analyzing information obtained from use of the panels. For example, the present invention provides data libraries comprising data collected from detection assay testing. For example, in some embodiments, the data libraries contain data obtained from an assay similar to at least one assay shown in FIG. 96, and in U.S. application Ser. No. 10/035,833 filed Dec. 27, 2001 and which is expressly incorporated by reference herein in its entirity. In some embodiments, the data libraries contain information obtained from greater than about 100 different assays (e.g., 100, 101, 102, . . . , 130, . . . , 500, . . . ). In some embodiments, data libraries include test result data including, but not limited to, the presence or absence of a mutation in nucleic acid from a sample, allele frequency information, quantitation data, and disease correlation data. In,some preferred embodiments, the data libraries also provide information correlated to the test result data including, but not limited to, an identity of a testing facility, detection assay components used to generate the data, other related detection assay components, reaction conditions, the identity of a user who requested the manufacture of the detection assay, date of detection assay use and/or testing, detection assay reliability information (e.g., determined the in silico methods of the present invention), information pertaining to the target sequence interrogated by the detection, information pertaining to clinical approval or requirements, and the like. In some embodiments, the present invention provides computer storage medium containing the above information and systems and methods for storing, accessing, and retrieving the information.

The present invention further provides methods for simultaneously detecting a plurality of polymorphisms (e.g., SNPs). For example, the present invention provides systems and methods for simultaneously detecting 100 or more polymorphism (100, . . . , 1000, . . . , 10,000, . . . , 100,000, . . . ). In some embodiments, the plurality of polymorphisms are detected in a single reaction sample (e.g., in a multiplex reaction). In some embodiments, the polymorphisms are present in genomic DNA and target sequences containing a single polymorphism are amplified prior to detection of the polymorphisms. In some embodiments, the amplification comprises PCR amplification. In some embodiments, amplification is carried out such that there is a 10⁵-10⁶-fold increase in copies of the target sequence.

The present invention further provides system and methods for developing detection assays based on the design of a pre-validated detection assay. For example, the present invention provides thousands of specific INVADER detections assays directed at different target nucleic acid sequences, as well as components that find use in other detection assay formats. In some embodiments, one or more components of these assays are used in or are used in the design of a different type of detection assay. For example, validated target sequences may be used as targets in other types of detection assay. Likewise, oligonucleotides that hybridize to target sequences may be used directly, or in the design of hybridization oligonucleotides for other types of detection assays. The present invention is not limited in the nature of the detection assay that is produced using information from the thousands of INVADER detection assays (e.g., assays described in FIG. 96, and in U.S. application Ser. No. 10/035,833 filed Dec. 27, 2001 and which is expressly incorporated by reference herein in its entirity). Such detection assays include, but are not limited to, hybridization methods and array technologies (e.g., Aclara BioSciences, Haywood, Calif.; Affymetrix, Santa Clara, Calif.; Agilent Technologies, Inc., Palo Alto, Calif.; Aviva Biosciences Corp., San Diego, Calif.; Caliper Technologies Corp., Palo Alto, Calif.; Celera, Rockville, Md.; CuraGen Corp., New Haven, Conn.; Hyseq Inc., Sunnyvale, Calif.; Illumina, Inc., San Diego, Calif.; Incyte Genomics, Palo Alto, Calif.; Motorola BioChip Systems; Nanogen, San Diego, Calif.; Orchid BioSciences, Inc., Princeton, N.J.; Applera Corp., Foster City, Calif.; Rosetta Inpharmatics, Kirkland, Wash.; and Sequenom, San Diego, Calif.); polymerase chain reaction; branched hybridization methods; enzyme mismatch cleavage methods; NASBA; sandwich hybridization methods; methods employing molecular beacons; ligase chain reactions, and the like.

The present invention relates to systems and methods for managing genetic information and medical records. For example, the present invention provides systems and methods for collecting, storing, and retrieving patient-specific genetic information from one or more electronic databases.

For example, in some embodiments, the present invention provides an electronic medical record comprising genetic information of a subject (e.g., single nucleotide polymorphism data of an animal or human patient) correlated to electronic medical history data of said subject. The present invention is not limited by the nature of the medical history data. Such data included, but is not limited to prescription data (e.g., data related to one or more drugs or other prescribed medical interventions of the subject, including drug identity, drug reaction data, allergies, risk assessment data, and multi-drug interaction data, billing code levels, order restrictions); information pertaining a physician visit (e.g., date and time of visit, identity of physicians, physician notes, diagnosis information, differential diagnosis information, patient location, patient status, order status, referral information); patient identification information (e.g., patient age, gender, race, insurance carrier, allergies, past medical history, family history, social history, religion, employer, guarantor, address, contact information, patient condition code); and laboratory information (e.g., labs, radiology, and tests).

In some embodiments, the genetic information comprises single nucleotide polymorphism data (e.g., data related to the presence of one or more single nucleotide polymorphisms in the genetic material of the subject, including, but not limited to, the identity of the polymorphisms, the location of the polymorphisms, medical conditions associated with the presence or absence of the polymorphisms, detection assays information) and/or information related to single nucleotide polymorphism data (e.g., allele frequency of the polymorphism in one or more populations).

In some embodiments, the single nucleotide polymorphism data comprises data derived from an in vitro diagnostic single nucleotide polymorphism detection assay. In some embodiments, the single nucleotide polymorphism data comprises data derived from a panel comprising a plurality of single nucleotide polymorphism detection assays. In some preferred embodiments, the panel comprises a detection assays that detects medically associated single nucleotide polymorphisms (e.g., single nucleotide polymorphisms associated with a disease). In some embodiments, the detection assays detect polymorphisms associated with one or more medically relevant subject areas including, but not limited to cardiovascular disease, oncology, immunology, metabolic disorders, neurological disorders, musculoskeletal disorders, endocrinology, and genetic disease. In some embodiments, the panel comprises a plurality of single nucleotide polymorphism detection assays associated with two or more diseases. In some embodiments, the panel comprises a plurality of single nucleotide polymorphism detection assays that detect polymorphisms in drug metabolizing enzymes.

In some embodiments, the single nucleotide polymorphism data comprises data derived from a plurality of in vitro diagnostic single nucleotide polymorphism detection assays. In some embodiments, the detection assays comprises two or more unique invasive cleavage assays (INVADER assay, Third Wave Technologies, Madison, Wis.). In some embodiments, one or more of the two or more unique invasive cleavage assays detected at least one single nucleotide polymorphism. In some embodiments, the single nucleotide polymorphism is associated with a medical condition. In some embodiments, the two or more unique invasive cleavage assays comprise at least 10 unique detection assays (e.g., 10, 11, 12, . . . , 100, . . . , 1000, . . . , 10,000, . . . , 50,000, . . . ).

In some embodiments, the single nucleotide polymorphism data is derived from an analyte-specific reagent assay. In some embodiments, the single nucleotide polymorphism data is derived from at least one clinically valid detection assay.

The electronic medical records of the present invention may be located on any number of computers or devices. For example, in some embodiments, the electronic medical record is contained in a computer system of a patient, an insurance company, a health care provider (e.g., a physician, a hospital, a clinic, a health maintenance organization), a government agency, and a drug retailer or drug wholesaler, or pharmaceutical company. In some embodiments, the electronic medical record is stored on a small device to be carried on or in a subject (e.g., a personal digital assistant, a MED-ALERT bracelet, a smart card, and an implanted data storage device such as those described in U.S. Pat. No. 5,499,626, herein incorporated by reference in its entirety).

In some embodiments, the electronic medical record comprises addition information, including, but not limited to, medical billing data, insurance claim data, and scheduling data.

The present invention also provides a computer system comprising the electronic medical records described herein. In some embodiments, the computer system is configured for receiving data from the Internet (e.g., e.g., single nucleotide polymorphism data or one or more SNP assay(s) result data). In some embodiments, the computer system comprises one or more hardware or software components configured to carry out a processing routine. For example, in some embodiments, a software application is configured to receive single nucleotide polymorphism data automatically via a communications network. In other embodiments, the computer system comprises a routine for categorizing data (e.g., by disease type, by patient type, by genetic loci, etc.). In some embodiments, the computer system comprises a routine for carrying out a bioinformatics analysis routine (e.g., as described elsewhere herein). In some embodiments, the computer system comprises a routine for carrying out a mathematical manipulation routine.

The present invention further provides a method for determining a correlation between a polymorphism (e.g., a SNP) and a phenotype, comprising: a) providing: samples from a plurality of subjects; medical records from the plurality of subjects, wherein the medical records contain information pertaining to a phenotype of the subjects; and detection assays that detect a polymorphism; b) exposing the samples to the detection assays under conditions such that the presence or absence of at least one polymorphism is revealed; and; c) determining a correlation between the at least one polymorphism and the phenotype of the subjects. In some embodiments, the plurality of subjects comprises 1000 or more subjects (e.g., 10,000 or more subjects). In some embodiments, the information pertaining to a phenotype comprises information pertaining to a disease. In other embodiments, the information pertaining to a phenotype comprises information pertaining to a drug interaction. In some embodiments, the medical record comprises an electronic medical record. While the present invention is not limited by the nature of the sample, in some preferred embodiments, the sample comprises a blood sample or a tissue biopsy.

The present invention also provides an electronic library comprising a plurality of electronic medical records for different subjects, each of the electronic medical records comprising, polymorphism data (e.g., single nucleotide polymorphism data) of the subject correlated to electronic medical history data of the subject. In some embodiments, the electronic medical history data comprises prescription data. In other embodiments, the prescription data comprises drug reaction data. In some embodiments, the single nucleotide polymorphism data comprises data derived from one or more in vitro diagnostic single nucleotide polymorphisms detection assays. In some embodiments, the single nucleotide polymorphism data comprises data derived from a panel, said panel comprising a plurality of single nucleotide polymorphisms detection assays. In some embodiments, the panel comprises detection assays that detect medically associated single nucleotide polymorphisms. In some embodiments, the panel comprises a plurality of single nucleotide polymorphisms detection assays that detect single nucleotide polymorphisms associated with a disease. In some embodiments, the panel comprises a plurality of detection assays that detect polymorphisms associated with one or more medically relevant subject areas including, but not limited to, cardiovascular disease, oncology, immunology, metabolic disorders, neurological disorders, musculoskeletal disorders, endocrinology, and genetic disease. In some embodiments, the panel comprises a plurality of single nucleotide polymorphism detection assays associated with two or more diseases. In some embodiments, the panel comprises a plurality of single nucleotide polymorphism detection assays that detect polymorphisms in drug metabolizing enzymes. In some embodiments, the single nucleotide polymorphism data comprises data derived from a plurality of in vitro diagnostic single nucleotide polymorphism detection assays for each said different subject. In some embodiments, the detection assays comprises two or more unique invasive cleavage assays. In some embodiments, the one or more of the two or more unique invasive cleavage assays detected at least one single nucleotide polymorphism. In some preferred embodiments, the at least one single nucleotide polymorphism is associated with a medical condition.

The present invention is not limited by the number of unique invasive cleavage assays used in the method. In some embodiments, the two or more unique invasive cleavage assays comprise at least 10 unique detection assays (e.g., at least 1000, 10,000, 35,000, or more).

In some embodiments, the single nucleotide polymorphism data for each of the different subjects is derived from an analyte-specific reagent assay. In some embodiments, the single nucleotide polymorphism data for each of the different subjects is derived from at least one clinically valid detection assay.

The present invention also provides computer systems comprising the electronic libraries. In some embodiments, the computer system is configured for securely receiving single nucleotide polymorphism data from the Internet. In some embodiments, the computer system further comprises a routine to receive single nucleotide polymorphism data for each of the different subjects automatically via a communications network. In some embodiments, the computer system further comprises a routine to receive single nucleotide polymorphism data for each the different subjects from nodes of a national, regional or world-wide communications network. In some embodiments, the computer system further comprises a software application for categorizing the data for the different subjects. In some embodiments, the computer system further comprises a software application for carrying out a bioinformatics analysis on said data for each said different subject.

The present invention provides systems and methods for acquiring and analyzing biological information. In particular, the present invention provides systems and methods for developing detection assays and for use of detection assays in basic research discovery to facilitate selection and development of clinical detection assays.

In some embodiments, the present invention provides methods of validating a detection assay, comprising: a) collecting test result data from a plurality of users, wherein the test result data is generated with one or more detection panels, and wherein the detection panels comprise a plurality of candidate detection assays configured for target detection; and b) processing at least a portion of the test result data such that at least one valid detection assay is identified from the plurality of candidate detection assays. In other embodiments, the method further comprises step c) marketing said valid detection assay as an Analyte-Specific Reagent or an In-Vitro Diagnostic. In certain embodiments, said marketing comprises selling and/or advertising. In other embodiments, the present invention provides methods of validating a detection assay, comprising: a) distributing one or more detection panels to a plurality of users, wherein the detection panels comprise a plurality of candidate detection assays configured for target detection; b) collecting test result data from at least a portion of the plurality of users, wherein the test result data is generated with the detection panels; and c) processing at least a portion of the test result data such that at least one valid detection assay is identified from the plurality of candidate detection assays. In other embodiments, the method further comprises step d) marketing said valid detection assay as an Analyte-Specific Reagent or an In-Vitro Diagnostic. In certain embodiments, said marketing comprises selling and/or advertising.

In particular embodiments, the plurality of detection assays comprise two or more unique detection assays (e.g. 10, . . . 50, . . . 100, . . . 1000, or more unique detection assays). In some embodiments, the plurality of detection assays comprise two or more unique INVADER assays (e.g. 10, . . . 50 . . . 100, . . . 1000, or more unique INVADER assays).

In certain embodiments, the methods of the present invention further comprise a distribution system, wherein the distributing is accomplished with the distribution system. In some embodiments, the distributing one or more detection panels to the plurality of users is at a reduced cost. In other embodiments, the distributing one or more detection panels to the plurality of users is at a subsidized cost. In still other embodiments, the distributing one or more detection panels to the plurality of users is at no cost.

In certain embodiments, prior to step a), the method further comprises the step of employing one or more of the plurality of candidate detection assays to discover at least one single nucleotide polymorphism. In particular embodiments, the plurality of detection assays comprise INVADER assays. In other embodiments, prior to step a), the method further comprises the step of utilizing one or more of the plurality of candidate detection assays to associate a single nucleotide polymorphism with a medical condition. In certain embodiments, the plurality of detection assays comprise INVADER assay components. In some embodiments, prior to step a), the method further comprises the step of utilizing one or more of the plurality of candidate detection assays, and computer aided analysis, to associate a single nucleotide polymorphism with a medical condition. In certain embodiments, the plurality of detection assays comprise INVADER assay components. In other embodiments, the INVADER assay components comprise an INVADER oligonucleotide, a probe, and a control target sequence. In particular embodiments, the plurality of detection assays comprise TAQMAN assay components (e.g. a probe and control target sequence).

In some embodiments, the one or more detection panels are configured for detecting a marker associated with a disease category. In certain embodiments, the disease category is selected from cardiovascular disease, cancer, autoimmune disease, metabolic disorders, neurological disease, musculoskeletal disorders, and endocrine related diseases.

In certain embodiments of the methods of the present invention, the plurality of users comprise researchers. In other embodiments, the plurality of users comprises at least 10 individual users. In some embodiments, the plurality of users comprises at least 200 individual users. In particular embodiments, the plurality of users comprises at least 500 individual users. In still other embodiments, the plurality of users comprises at least 1000 individual users. In particular embodiments, the plurality of users comprises at least 10,000 individual users.

In some embodiments of the methods of the present invention, the plurality of detection assays comprises at least 10 unique detection assays. In other embodiments, the plurality of detection assays comprises at least 1000 unique detection assays. In particular embodiments, the plurality of detection assays comprises at least 10,000 unique detection assays. In certain embodiments, the plurality of detection assays comprises at least 50,000 unique detection assays.

In particular embodiments, the method further comprises a step, after the processing step, of selling the at least one valid detection assay as an Analyte Specific Reagent (ASR). In some embodiments the method further comprises a step, after the processing step, of selling the at least one valid detection assay as an Analyte Specific Reagent (ASR) to an In-Vitro Diagnostic Manufacturer or to a non-clinical laboratory. In additional embodiments, the method further comprises a step, after the processing step, of selling the at least one valid detection assay as an In-Vitro Diagnostic.

In some embodiments, the test result data comprises raw assay data. In other embodiments, test result data comprises analyzed assay data. In certain embodiments, the test result data comprises both raw assay data and analyzed assay data. In particular embodiments, the test result data comprises data resulting from testing of at least separate samples (e.g. at least 1000, at least 10,000, or at least 100,000 separate samples).

In certain embodiments, the collecting comprises receiving the test result data from at least a portion of the plurality of users over a communications network (e.g. Internet or World Wide Web). In some embodiments, the collecting further comprises storing the test result data in a database. In particular embodiments, the database is part of a computer system of a service provider. In certain embodiments, the collecting comprises receiving the test result data over the Internet. In some embodiments, the collecting comprises retrieving the test result data from a user's computer system over a communication network. In additional embodiments, the user's computer system comprises a software application configured to receive the test result data. In some embodiments, the software application is further configured to transmit the test result data automatically via a communications network.

In some embodiments, the processing comprises categorizing the test result data (e.g. arranging the data according to unique detection assay and/or type of medical condition associated with detection of a target). In other embodiments, the processing comprises in silico analysis. In certain embodiments, the processing comprises computer aided analysis of the test result data. In additional embodiments, the processing comprises mathematical manipulation of the test result data. In further embodiments, the processing comprises comparing the test result data to a substantially equivalent predicate assay. In particular embodiments, the processing comprises mathematical manipulation of the test result data, and comparing the test result data to a substantially equivalent predicate assay.

In certain embodiments, at least one valid detection assay is identified as a result of being substantially equivalent to a predicate assay. In some embodiments, processing at least a portion of the test result data generates assay validation information.

In some embodiments, the methods of the present invention further comprise step e) submitting the assay validation information to a government body charged with approving products for clinical use. In certain embodiments, the government body is the Food and Drug Administration. In particular embodiments, the assay validation information is part of a 510(k) application that is submitted to the Food and Drug Administration. In other embodiments, the methods of the present invention further comprise a step of receiving approval from the Food and Drug Administration to market the at least one valid detection assay as an FDA approved In-Vitro diagnostic assay. In additional embodiments, the FDA approved In-Vitro diagnostic assay is a predicate for determining substantially equivalency for other In-Vitro diagnostic assays.

In some embodiments, the target is a single nucleotide polymorphism (e.g. in a DNA or RNA molecule). In other embodiments, the target is RNA (e.g. such that RNA expression can be quantitated).

The present invention also provides a method of developing an in-vitro diagnostic DNA or RNA analysis product comprising, running an assay through a product development funnel, in which the assay that enters the product development funnel is substantially similar to the in-vitro diagnostic DNA or RNA analysis product. In some embodiments, the assay is an assay to detect a single nucleotide polymorphism. In some preferred embodiments, the product development funnel optionally comprises one or more of the following: a discovery portion, a medically associated portion, an analyte-specific reagent portion, and an in-vitro diagnostic portion. In some embodiments, the assay comprises a chromosome specific assay. In some embodiments, the method further comprises the step of using a panel, wherein the panel comprises the assay. In other embodiments, the panel comprises a whole genome panel.

In some embodiments, the medically associated portion of the funnel comprises a panel organized by disease. In some preferred embodiments, the panel organized by disease is selected from the group consisting of a cardiovascular disease panel, an oncology panel, an immunology panel, a metabolic disorders panel, a neurological disorders panel, a musculoskeletal disorders panel, an endocrinology panel, and a genetic disease panel.

In some embodiments, the method further comprises the step of using a panel, wherein the panel is a panel for a multiplicity of disease states and/or wherein the panel comprises a drug metabolizing enzyme panel.

The present invention further provides a method of increasing revenue and/or a profit margin from the development of an in vitro diagnostic DNA or RNA analysis product comprising channeling an assay through a product development funnel, in which the assay is substantially similar to the in vitro diagnostic DNA or RNA analysis product. In some embodiments, the in vitro DNA or RNA analysis product comprises an FDA approved product. In some preferred embodiments, the product development funnel has an ingress and an egress, wherein the assay is one of at least several thousand assays which enter the ingress. In other embodiments, the assay is one of about several hundred assays that exit the egress as the in vitro diagnostic DNA or RNA analysis product.

The present invention further provides a method of identifying single nucleotide polymorphisms comprising providing: 1) a plurality of samples comprising genomic DNA from a first individual and four or more additional individuals, each of the first and four or more additional individuals having genomic DNA comprising a first region, said first individual having a first single nucleotide polymorphism in the first region, 2) at least one detection reagent capable of generating a signal; and 3) at least one oligonucleotide probe designed to cause the detection reagent to generate a signal following contact of the probe with a portion of the first region of the genomic DNA of the first individual; contacting each of the genomic DNA samples with the oligonucleotide probe under conditions such that a signal is detected for the genomic DNA of the first individual; identifying at least one of the four or more additional individuals for which no signal is detected, thereby identifying a negative-tested individual; and assaying the first region of the negative-tested individual under conditions such that a second single nucleotide polymorphism is revealed in the first region of the genomic DNA of the negative-tested individual in addition to the first single nucleotide polymorphism, wherein the first individual lacks the second single nucleotide polymorphism. In some embodiments, the method further provides a second oligonucleotide probe designed to cause the detection reagent to generate a signal following contact of the probe with a portion of the first region of the genomic DNA of the negative-tested individual, wherein the second oligonucleotide probes is contacted with the genomic DNA sample of the negative-tested individual. The second probe may be used concurrently with the first probe or may be used after the first probe (e.g., experiments conducted with the first probe may lead to the design of a second probe e.g., using the systems and methods of the present invention). The method may also include identifying negative detection assay results that are the result of one or more individuals lacking the first single nucleotide polymorphism.

### DESCRIPTION OF THE FIGURES

The following figures form part of the present specification and are included to further demonstrate certain aspects and embodiments of the present invention. The invention may be better understood by reference to one or more of these figures in combination with the description of specific embodiments presented herein.

FIG. 1 shows a general overview of the systems of the present invention.

FIGS. 2a-2f show various embodiments of INVADER LOCATOR computer interface displays.

FIG. 3 shows an overview of in silico analysis in some embodiments of the present invention.

FIG. 4 shows an overview of information flow for the design and production of detection assays in some embodiments of the present invention.

FIG. 5 shows how the in silico processes of the present invention allow information to be processed to generate useful detection panels.

FIG. 6 shows one embodiment of the INVADER detection assay.

FIG. 7 shows a computer display of an INVADERCREATOR Order Entry screen.

FIG. 8 shows a computer display of an INVADERCREATOR Multiple SNP Design Selection screen.

FIG. 9 shows a computer display of an INVADERCREATOR Designer Worksheet screen.

FIG. 10 shows a computer display of an INVADERCREATOR Output Page screen.

FIG. 11 shows a computer display of an INVADERCREATOR Printer Ready Output screen.

FIG. 12 A-12R show various SNP INVADER CREATOR (SIC) computer interface displays.

FIGS. 13A-13Q show various RIC INVADERCREATOR computer interface displays.

FIGS. 14a-14f show various TIC INVADER CREATOR computer interface displays.

FIG. 15 shows an input target sequence and the result of processing this sequence with systems and routines of the present invention.

FIG. 16 shows an example of a basic work flow for highly multiplexed PCR using the INVADER Medically Associated Panel.

FIG. 17 shows a flow chart outlining the steps that may be performed in order to generate a primer set useful in multiplex PCR.

FIGS. 18-22 show sequences used and data generated in connection with PCR Primer Design Example 1.

FIGS. 23-30 show sequences used and data generated in connection with Example 2.

FIG. 31 shows certain PCR primers useful for amplifying various regions of CYP2D6.

FIG. 32 shows one protocol for Multiplex PCR optimization according to the present invention.

FIG. 33 illustrates a perspective view of an exemplary synthesizer.

FIG. 34 illustrates a cross-sectional view of an exemplary synthesizer.

FIG. 35 illustrates a perspective view of a cartridge, chamber bowl and chamber seal of the present invention.

FIG. 36 illustrates a detailed view of an exemplary cartridge.

FIG. 37 illustrates an exemplary drain plate:

FIG. 38A illustrates a top view of one embodiment of a drain plate. FIG. 38B illustrates a top view of another embodiment of a drain plate gasket.

FIG. 39 illustrates a side view of a drain plate gasket situated between a cartridge and a drain plate.

FIG. 40 illustrates a cross-sectional view of a waste tube system.

FIG. 41 illustrates a chamber bowl with chamber drain.

FIGS. 42A-C illustrate different embodiments of energy input components 95 and mixing components 96.

FIGS. 43A-B illustrate different combinations of energy input components 95 and mixing components 96.

FIG. 44 illustrates one embodiment of a synthesis column.

FIG. 45 illustrates a computer system coupled to a synthesizer.

FIGS. 46A-C illustrate 3 cross-sectional detailed views of different embodiments of a cartridge, drain plate, drain plate gasket, receiving hole of cartridge, and synthesis column.

FIG. 47A and 47B illustrate embodiments of reagent dispense stations.

FIG. 48A illustrates a synthesizer having a ventilation opening in a lid enclosure.

FIGS. 48B and 48C illustrate a synthesizer having ventilation tubing attached to a ventilation opening in a lid enclosure.

FIGS. 49A-C illustrate synthesizers having ventilated workspaces.

FIGS. 50A and 50B provide cross sectional views of an exemplary synthesizer having a lid enclosure 102, and illustrate air flow 109 toward the ventilation tubing 103 when the lid enclosure 102 is in a closed or opened position, respectively.

FIGS. 51A and 51B provide cross sectional views of an exemplary synthesizer having a primarily enclosed space in a base 2, and illustrate air flow 109 toward the ventilation tubing 103 when the lid enclosure 102 is in a closed or opened position, respectively.

FIG. 52 illustrates a synthesizer 1, a robotic means 92, a cleave and deprotect component 93 and a purification component 94.

FIG. 53 shows a schematic diagram of a polymer synthesizer of the present invention.

FIG. 54A shows a side view of a reagent dispenser (2). FIG. 54B shows a cross-sectional view of a reagent dispenser (2).

FIGS. 55A and 55B show a preferred embodiment of the reagent dispenser (2), wherein the outer surface of the delivery channel (9) contains first (13) and second (14) ring seals configured to form an airtight or substantially airtight seal with one or more points on the interior surface of a synthesis column (15) or other reaction chamber (e.g., with reaction chambers present in a synthesizer or a cleavage and deprotection component).

FIG. 56 shows a solvent delivery component in one embodiment of the present invention.

FIG. 57 shows a waste storage and purge component in one embodiment of the present invention.

FIGS. 58A-K show flow charts depicting the integrated data and process flows employed in the oligonucleotide production systems of the present invention.

FIG. 59A-D show various protocols for high throughput, automated genotyping.

FIG. 60A-60H various embodiments of the cleave and deprotect devices, and components thereof, of the present invention.

FIG. 61 shows one embodiment of a data management system of the present invention.

FIG. 62 shows another embodiment of a data management system of the present invention.

FIG. 63 shows a computer display of an association database.

FIG. 64 shows a computer display of a Microsoft Excel worksheet having data received by export from an association database.

FIG. 65 shows a computer display of a plate viewer.

FIG. 66 shows a computer display of a data viewer.

FIG. 67 shows a computer display of allele caller results, having SNP results data displayed in the cells.

FIG. 68 shows a computer display of allele caller results, having analyzed input assay data (in this example, a calculated ratio) displayed in the cells.

FIG. 69 shows a computer display of a Microsoft Excel worksheet having SNP results data received by export from an allele caller.

FIG. 70 shows a graph demonstrating the ability of the INVADER assay to detect mutations in the APOC4 gene in pooled samples.

FIG. 71 shows a graph demonstrating the ability of the INVADER assay to detect mutations in the CFTR gene in pooled samples.

FIGS. 72-75 show graphs of the results of experiments described in Pooled Sample-Example 3.

FIG. 76A shows data measuring allele signals in INVADER assays for detection of alleles comprising the indicated percentages of the number of copies of each locus.

FIG. 76B shows an Excel graph comparing theoretical allele frequencies to allele frequencies calculated from the INVADER assay data shown in FIG. 5A.

FIG. 77 shows an Excel graph and data comparing actual and calculated allele frequencies for each of 8 SNP loci detected in pooled genomic DNA from 8 different individuals.

FIG. 78 shows an Excel graph and data showing calculated allele frequencies compared to fold-over-zero minus 1 (FOZ−1) measurements for SNP locus 132505 in genomic DNAs having different mixtures of these alleles.

FIG. 79 shows an Excel graph and data showing calculated allele frequencies compared to fold-over-zero minus 1 (FOZ−1) measurements for SNP locus 131534 in genomic DNAs having different mixtures of these alleles.

FIGS. 80A-80C show the sequences of the probes configured for use in the assays described in Pooled Sample-Example 4 and synthetic targets for each allele. “Y” indicates an amine blocking group. The polymorphism and the dye that will be detected for each probe, when used in the exemplary assay configurations described in Example 4, are indicated.

FIG. 81 shows an overview of the integration of components of the systems and methods of the present invention.

FIG. 82 shows identified p450 2D6 polymorphisms.

FIG. 83 shows CYP2D6 specific PCR amplification.

FIG. 84 depicts biplex signal detection using INVADER assays to detect CYP2D6.

FIGS. 85 and 86 show the results of an INVADER assay screen of 175 individuals for various CYP2D6 polymorphisms.

FIG. 87 shows the minor allele frequency by population for various SNP consortium/Third Wave Technologies SNPs.

FIG. 88 shows a schematic summary of the flow of detection assay development in the present invention from research products to clinical products.

FIG. 89 shows a schematic summary of the discovery phase of the diagram shown in FIG. 88.

FIG. 90 shows a schematic summary of the development of potential clinical markers phase of the diagram shown in FIG. 88.

FIG. 91 shows exemplary detection assay products from each phase of the diagram shown in FIG. 88.

FIG. 92 shows business revenue generation from products from each phase of the diagram shown in FIG. 88. The arrows showing revenue/margin per detection assay are not quantitative, but simply show a qualitative increase for each layer of the funnel.

FIG. 93 shows a flow chart depicting a disease associated assay development process.

FIG. 94 shows an overview of an ASR Fast Track Process.

FIG. 95 shows a flow chart depicting a process for identifying “Super SNPs.”

FIG. 96 shows INVADER assay components for detecting polymorphisms in certain genes.

FIG. 97A-97D shows various steps in the quality control assessment methods and protocols of the present invention.

FIG. 98 shows a general overview of the oligonucleotide production and processing systems of the present invention.

FIG. 99 shows various genes, what role they play, the substrate they act on, and the number of polymorphisms in these genes. The polymorphisms in these genes may be tested by the detection assay described herein. In this regard, a subject found to have a polymorphism reducing or eliminating the activity of these genes may, for example, select drugs that will not harm the subject, and that are likely to be effective.

FIG. 100 shows oligonucleotides for an exemplary INVADER assay for detecting UGT1A1*6.

FIG. 101 shows oligonucleotides for an exemplary INVADER assay for detecting UGT1A1*27.

FIG. 102 shows oligonucleotides for an exemplary INVADER assay for detecting UGT1A1*28.

FIG. 103 shows set of nine polymorphisms in human UGT1A1.

FIG. 104 shows exemplary detection assays (INVADER assays) for the nine UGT1A1 polymorphisms shown in FIG. 103.

FIG. 105 shows exemplary detection probes for detection of UGT1A1*28 alleles. “Hex” indicates a hexanediol 3′ blocking group.

FIG. 106 shows exemplary detection probes for detection of UGT1A1*28 alleles. “Hex” indicates a hexanediol 3′ blocking group.

FIG. 107 shows an Excel graph showing detection of UGT1A1*28 wild-type (WT), insertion (Ins) alleles in samples of genomic DNA.

FIG. 108 shows Excel graphs of detection of UGT1A1*28 WT and Ins alleles in reactions having different amounts of target DNA.

FIG. 109 shows exemplary INVADER assay configurations for TA5, TA6, TA7, and TA8 UGT1A1*28 detection.

FIG. 110 shows an exemplary INVADER assay configuration for TA5 UGT1A1*28 detection.

FIG. 111 shows an exemplary INVADER assay configuration for TA6 UGT1A1*28 detection.

FIG. 112 shows an exemplary INVADER assay configuration for TA7 UGT1A1*28 detection.

FIG. 113 shows an exemplary INVADER assay configuration for TA8 UGT1A1*28 detection.

FIG. 114 shows an exemplary INVADER assay design for an internal control (Alpha Actin) that may be used with UGT detection assays.

FIG. 115 shows certain results of the UGT Example 2.

FIG. 116 shows certain results of the UGT Example 2.

FIG. 117 shows certain colon cancer management protocols.

FIG. 118 shows certain colon cancer management protocols.

FIG. 119 shows certain colon cancer management protocols.

FIG. 120 shows oligonucleotides for an exemplary INVADER assay for detecting UGT1A1*27.

FIG. 121 shows oligonucleotides for an exemplary INVADER assay for detecting UGT1A1*28.

FIG. 122 shows certain PCR primers useful for amplifying various regions of CYP2D6.

FIG. 123 shows a schematic representation of the CYP2D6 genomic region and one embodiment of a triplex PCR strategy. A. The position of CYP2D6 in relation-to its 2 pseudogenes CYP2D7 and CYP2D8. B. The positions of polymorphisms found within or bordering the nine CYP2D6 exons. The relative frequency of the different polymorphisms is indicated by the length of the arrow. Solid arrows indicate non-synonymous polymorphisms and hashed arrows synonymous. The position and base change of each polymorhpism is indicated at the end if the arrow. The asterisk (*) below the arrows indicates 11 polymorphisms investigated in this study. The position and size of the three PCR products in this embodiment of a triplex PCR reaction is indicated below the exons. C. An example of PCR products generated in a triplex PCR reaction as visualized on an agarose gel.

FIG. 124 shows a table of oligonucleotides used for amplification and INVADER assay detection of CYP 2D6 alleles.

FIG. 125 shows representative data from analysis of exemplary CYP 2D6 alleles using the methods and compositions of the present invention. Each allele tested is indicated at the top of each panel 1 through 5.

FIG. 126. Summary of the data from a screen of 174 DNAs with 11 CYP2D6 Invader genotyping assays

FIG. 127. CYP2D6 haplotype predictions from 175 genomic samples using the Expectation maximization algorithm implemented on the Arlequin genetic software.

FIG. 128. Compound CYP2D6 haplotypes for 171 DNAs genotyped by the Invader system and categorized in to number of functional alleles.

FIG. 129 shows various CYP2CP polymorphism targets, and INVADER assay oligonucleotides for detecting polymorphisms in these targets.

FIG. 130 shows various genes, what role they play, the substrate they act on, and the number of polymorphisms in these genes. The polymorphisms in these genes may be tested by the detection assay described herein. In this regard, a subject found to have a polymorphism reducing or eliminating the activity of these genes may, for example, select drugs that will not harm the subject, and that are likely to be effective.

FIG. 131 shows a table useful in correlating particular genotypes with particular phenotypes, and further correlating particular drugs with particular diseases. In particular, this figure shows many genes and the pathways and/or function for these genes. This figure also shows various diseaes and the pathways typically associated with these diseases (allowing one to refer back to genes that in this figure that may then be involved with these diseases). This figure further shows many polymorphisms that are present in certain genes (thereby allowing one to identify polymorphisms associated with a gene that is associated with a disease). Finally, this figure provides a list of therapeutic agents and the action and/or disease the therapeutic agent is used for. In this regard, one employing this figure to identify polymorphisms that could be tested, for example, for in a patient with a particular disease prior to administering a particular therapeutic agent to the patient. This figure is also useful in combination with Tables 10, 11, 12 and FIG. 96 in order to personalize drug therapy for a patient.

### DEFINITIONS

To facilitate an understanding of the present invention, a number of terms and phrases are defined below:

As used herein, the terms “solid support” or “support” refer to any material that provides a solid or semi-solid structure with which another material can be attached. Such materials include smooth supports (e.g., metal, glass, plastic, silicon, and ceramic surfaces) as well as textured and porous materials. Such materials also include, but are not limited to, gels, rubbers, polymers, and other non-rigid materials. Solid supports need not be flat. Supports include any type of shape including spherical shapes (e.g., beads). Materials attached to solid support may be attached to any portion of the solid support (e.g., may be attached to an interior portion of a porous solid support material). Preferred embodiments of the present invention have biological molecules such as nucleic acid molecules and proteins attached to solid supports. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linker group.” Such spacer molecules are molecules that have a first portion that attaches to the biological material and a second portion that attaches to the solid support. Thus, when attached to the solid support, the spacer molecule separates the solid support and the biological materials, but is attached to both.

As used herein, the term “derived from a different subject,” such as samples or nucleic acids derived from a different subjects refers to a samples derived from multiple different individuals. For example, a blood sample comprising genomic DNA from a first person and a blood sample comprising genomic DNA from a second person are considered blood samples and genomic DNA samples that are derived from different subjects. A sample comprising five target nucleic acids derived from different subjects is a sample that includes at least five samples from five different individuals. However, the sample may further contain multiple samples from a given individual.

As used herein, the term “treating together,” when used in reference to experiments or assays, refers to conducting experiments concurrently or sequentially, wherein the results of the experiments are produced, collected, or analyzed together (i.e., during the same time period). For example, a plurality of different target sequences located in separate wells of a multiwell plate or in different portions of a microarray are treated together in a detection assay where detection reactions are carried out on the samples simultaneously or sequentially and where the data collected from the assays is analyzed together.

The terms “assay data” and “test result data” as used herein refer to data collected from performance of an assay (e.g., to detect or quantitate a gene, SNP or an RNA). Test result data may be in any form, i.e., it may be raw assay data or analyzed assay data (e.g., previously analyzed by a different process). Collected data that has not been further processed or analyzed is referred to herein as “raw” assay data (e.g., a number corresponding to a measurement of signal, such as a fluorescence signal from a spot on a chip or a reaction vessel, or a number corresponding to measurement of a peak, such as peak height or area, as from, for example, a mass spectrometer, HPLC or capillary separation device), while assay data that has been processed through a further step or analysis (e.g., normalized, compared, or otherwise processed by a calculation) is referred to as “analyzed assay data” or “output assay data”.

As used herein, the term “database” refers to collections of information (e.g., data) arranged for ease of retrieval, for example, stored in a computer memory. A “genomic information database” is a database comprising genomic information, including, but not limited to, polymorphism information (i.e., information pertaining to genetic polymorphisms), genome information (i.e., genomic information), linkage information (i.e., information pertaining to the physical location of a nucleic acid sequence with respect to another nucleic acid sequence, e.g., in a chromosome), and disease association information (i.e., information correlating the presence of or susceptibility to a disease to a physical trait of a subject, e.g., an allele of a subject). “Database information” refers to information to be sent to databases, stored in a database, processed in a database, or retrieved from a database. “Sequence database information” refers to database information pertaining to nucleic acid sequences. As used herein, the term “distinct sequence databases” refers to two or more databases that contain different information than one another. For example, the dbSNP and GenBank databases are distinct sequence databases because each contains information not found in the other.

As used herein, the terms “centralized control system” or “centralized control network” refer to information and equipment management systems (e.g., a computer processor and computer memory) operable linked to a module or modules of equipment (e.g., DNA synthesizers).

As used herein, the term “oligonucleotide synthesizer component” refers to a component of a system that is capable of synthesizing oligonucleotides (e.g., a oligonucleotide synthesizers). In some embodiments, the oligonucleotide synthesizer component comprises a plurality of oligonucleotide synthesizers that are operably linked.

As used herein, the term “oligonucleotide processing component” refers to a component of a system capable of processing of oligonucleotides post-synthesis. Examples of oligonucleotide processing stations include, but are not limited to, purification stations, dry-down stations, cleavage and deprotection stations, desalting stations, dilute and fill stations, and quality control stations.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refers to a device that is able to read a program from a computer memory (e.g., ROM or other computer memory) and perform a set of steps according to the program.

As used herein the term “oligonucleotide specification information” refers to any information used during the production of an oligonucleotide. Examples of oligonucleotide specification information includes, but is not limited to, sequence information, end-user (e.g., customer) information, and concentration information (e.g., the final concentration desired by the end-user).

As used herein the term “corresponding oligonucleotides” is used to refer to oligonucleotides that differ in at least one characteristic (e.g., sequence, purity, required buffer, required salt concentration) and that are to be provided together (e.g., in an INVADER assay, the INVADER oligonucleotide and Primary Probe are ‘corresponding oligonucleotides’).

As used herein, the term “divergent production” refers to the production of corresponding oligonucleotides employing at least two manufacturing stations, where a first corresponding oligonucleotide is never processed by at least one manufacturing station that is used to process a corresponding oligonucleotide.

As used herein the term “set of oligonucleotides” means at least two oligonucleotides that differ in at least one characteristic (e.g., sequence, purity, required buffer, required salt concentration).

As used herein the term “purified sample,” as in a purified oligonucleotide sample, refers to a sample where the full-length oligonucleotide in a sample is the predominate species of oligonucleotide. For example, in some embodiments, at least 90%, preferably 95%, and more preferably 99% of oligonucleotides in a sample are full-length oligonucleotides.

As used herein, the terms “SNP,” “SNPs” or “single nucleotide polymorphisms” refer to single base changes at a specific location in an organism's (e.g., a human) genome. “SNPs” can be located in a portion of a genome that does not code for a gene. Alternatively, a “SNP” may be located in the coding region of a gene. In this case, the “SNP” may alter the structure and function of the RNA or the protein with which it is associated.

As used herein, the term “allele” refers to a variant form of a given sequence (e.g., including but not limited to, genes containing one or more SNPs). A large number of genes are present in multiple allelic forms in a population. A diploid organism carrying two different alleles of a gene is said to be heterozygous for that gene, whereas a homozygote carries two copies of the same allele.

As used herein, the term “linkage” refers to the proximity of two or more markers (e.g., genes) on a chromosome.

As used herein, the term “allele frequency” refers to the frequency of occurrence of a given allele (e.g., a sequence containing a SNP) in given population (e.g., a specific gender, race, or ethnic group). Certain populations may contain a given allele within a higher percent of its members than other populations. For example, a particular mutation in the breast cancer gene called BRCA1 was found to be present in one percent of the general Jewish population. In comparison, the percentage of people in the general U.S. population that have any mutation in BRCA1 has been estimated to be between 0.1 to 0.6 percent. Two additional mutations, one in the BRCA1 gene and one in another breast cancer gene called BRCA2, have a greater prevalence in the Ashkenazi Jewish population, bringing the overall risk for carrying one of these three mutations to 2.3 percent.

As used herein, the term “in silico analysis” refers to analysis performed using computer processors and computer memory. For example, “insilico SNP analysis” refers to the analysis of SNP data using computer processors and memory.

As used herein, the term “genotype” refers to the actual genetic make-up of an organism (e.g., in terms of the particular alleles carried at a genetic locus). Expression of the genotype gives rise to an organism's physical appearance and characteristics-the “phenotype.”

As used herein, the term “locus” refers to the position of a gene or any other characterized sequence on a chromosome.

As used herein the term “disease” or “disease state” refers to a deviation from the condition regarded as normal or average for members of a species, and which is detrimental to an affected individual under conditions that are not inimical to the majority of individuals of that species (e.g., diarrhea, nausea, fever, pain, and inflammation etc).

As used herein, the term “treatment” in reference to a medical course of action refer to steps or actions taken with respect to an affected individual as a consequence of a suspected, anticipated, or existing disease state, or wherein there is a risk or suspected risk of a disease state. Treatment may be provided in anticipation of or in response to a disease state or suspicion of a disease state, and may include, but is not limited to preventative, ameliorative, palliative or curative steps. The term “therapy” refers to a particular course of treatment.

The term “gene” refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5! untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in corresponding portions of the produced RNAs (e.g., hnRNAs, mRNAs, rRNAs, tRNAs).

Where the phrase “amino acid sequence” is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

DNA and RNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs, the two splice variants are therefore substantially homologous to such a probe and to each other.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions!” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100·μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent-to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50X Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polynucleotides, the term “substantial identity” denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a splice variant of the full-length sequences.

As applied to polypeptides, the term “substantial identity” means that two peptide sequences, when optimally aligned, such as by the programs GAP or BEST FIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate-its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” or “hybridization probe” refers to an oligonucleotide (i.e., a sequence of nucleotides), whether occurring naturally as in a purified restriction digest or produced synthetically, recombinantly or by PCR amplification, that is capable of hybridizing, at least in part, to another oligonucleotide of interest. A probe may be single-stranded or double-stranded. Probes are useful in the detection, identification and isolation of particular sequences. In some preferred embodiments, probes used in the present invention will be labeled with a “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target” refers to a nucleic acid sequence or structure to be detected or characterized.

As used herein, the term “polymerase chain reaction” (“PCR”) refers to the method of K. B. Mullis (See e.g., U.S. Pat. Nos. 4,683,195, 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the term “recombinant DNA molecule” as used herein-refers to a DNA molecule that is comprised of segments of DNA joined together by means of molecular biological techniques.

As used herein, the term “antisense” is used in reference to RNA sequences that are complementary to a specific RNA sequence (e.g., mRNA). The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acids encoding a polypeptide include, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 11, . . . , 20, . . . ).

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of labeled antibodies.

The term “test compound” refers to any chemical entity, pharmaceutical, drug, and the like that are tested in an assay (e.g., a drug screening assay) for any desired activity (e.g., including but not limited to, the ability to treat or prevent a disease, illness, sickness, or disorder of bodily function, or otherwise alter the physiological or cellular status of a sample). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass. spectrometry or surface enhanced Raman spectrometry; a system such as gel or capillary electrophoresis or gel exclusion chromatography; or other detection system known in the art, or combinations thereof.

As used herein, the term “distribution system” refers to systems capable of transferring and/or delivering materials from one entity to another or one location to another. For example, a distribution system for transferring detection panels from a manufacturer or distributor to a user may comprise, but is not limited to, a packaging department, a mail room, and a mail delivery system. Alternately, the distribution system may comprise, but is not limited to, one or more delivery vehicles and associated delivery personnel, a display stand, and a distribution center. In some embodiments of the present invention interested parties (e.g., detection panel manufactures) utilize a distribution system to transfer detection panels to users at no cost, at a subsidized cost, or at a reduced cost.

As used herein, the term “at a reduced cost” refers to the transfer of goods or services at a reduced direct cost to the recipient (e.g. user). In some embodiments, “at a reduced cost” refers to transfer of goods or services at no cost to the recipient.

As used herein, the term “at a subsidized cost” refers to the transfer of goods or services, wherein at least a portion of the recipient's cost is deferred or paid by another party. In some embodiments, “at a subsidized cost” refers to transfer of goods or services at no cost to the recipient.

As used herein, the term “at no cost” refers to the transfer of goods or services with no direct financial expense to the recipient. For example, when detection panels are provided by a manufacturer or distributor to a user (e.g. research scientist) at no cost, the user does not directly pay for the tests.

The term “detection” as used herein refers to quantitatively or qualitatively identifying an analyte (e.g., DNA, RNA or a protein) within a sample. The term “detection assay” as used herein refers to a kit, test, or procedure performed for the purpose of detecting an analyte nucleic acid within a sample. Detection assays produce a detectable signal or effect when performed in the presence of the target analyte, and include but are not limited to assays incorporating the processes of hybridization, nucleic acid cleavage (e.g., exo- or endonuclease), nucleic acid amplification, nucleotide sequencing, primer extension, or nucleic acid ligation.

As used herein, the term “functional detection oligonucleotide” refers to an oligonucleotide that is used as a component of a detection assay, wherein the detection assay is capable of successfully detecting (i.e., producing a detectable signal) an intended target nucleic acid when the functional detection oligonucleotide provides the oligonucleotide component of the detection assay. This is in contrast to a non-functional detection oligonucleotides, which fail to produce a detectable signal in a detection assay for the particular target nucleic acid when the non-functional detection oligonucleotide is provided as the oligonucleotide component of the detection assay. Determining if an oligonucleotide is a functional oligonucleotide can be carried out experimentally by testing the oligonucleotide in the presence of the particular target nucleic acid using the detection assay.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “hypertext system” refers to a computer-based informational system in which documents (and possibly other types of data entities) are linked together via hyperlinks to form a user-navigable “web.”

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein, the terms “World Wide Web” -or “web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms. “Web” and “World Wide Web” are intended to encompass future markup languages and transport protocols that may be used in place of (or in addition to) HTML and HTTP.

As used herein, the term “web site” refers to a computer system that serves informational content over a network using the standard protocols of the World Wide Web. Typically, a Web site corresponds to a particular Internet domain name and includes the content associated with a particular organization. As used herein, the term is generally intended to encompass both (i) the hardware/software server components that serve the informational content over the network, and (ii) the “back end” hardware/software components, including any non-standard or specialized components, that interact with the server components to perform services for Web site users.

As used herein, the term “HTML” refers to HyperText Markup Language that is a standard coding convention and set of codes for attaching presentation and linking attributes to informational content within documents. HTML is based on SGML, the Standard Generalized Markup Language. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. Additionally, in specifying how the Web browser is to display the document, HTML tags can be used to create links to other Web documents (commonly referred to as “hyperlinks”).

As used herein, the term “XML” refers to Extensible Markup Language, an application profile that, like HTML, is based on SGML. XML differs from HTML in that: information providers can define new tag and attribute names at will; document structures can be nested to any level of complexity; any XML document can contain an optional description of its grammar for use by applications that need to perform structural validation. XML documents are made up of storage units called entities, which contain either parsed or unparsed data. Parsed data is made up of characters, some of which form character data, and some of which form markup. Markup encodes a description of the document's storage layout and logical structure. XML provides a mechanism to impose constraints on the storage layout and logical structure, to define constraints on the logical structure and to support the use of predefined storage units. A software module called an XML processor is used to read XML documents and provide access to their content and structure.

As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL.

As used herein, the term “URL” refers to Uniform Resource Locator that is a unique address that fully specifies the location of a file or other resource on the Internet. The general format of a URL is protocol://machine address:port/path/filename. The port specification is optional, and if none is entered by the user, the browser defaults to the standard port for whatever service is specified as the protocol. For example, if HTTP is specified as the protocol, the browser will use the HTTP default port of 80.

As used herein, the term “PUSH technology” refers to an information dissemination technology used to send data to users over a network. In contrast to the World Wide Web (a “pull” technology), in which the client browser must request a Web page before it is sent, PUSH protocols send the informational content to the user computer automatically, typically based on information pre-specified by the user.

As used herein, the term “communication network” refers to any network that allows information to be transmitted from one location to another. For example, a communication network for the transfer of information from one computer to another includes any public or private network that transfers information using electrical, optical, satellite transmission, and the like. Two or more devices that are part of a communication network such that they can directly or indirectly transmit information from one to the other are considered to be “in electronic communication” with one another. A computer network containing multiple computers may have a central computer (“central node”) that processes information to one or more sub-computers that carry out specific tasks (“sub-nodes”). Some networks comprises computers that are in “different geographic locations” from one another, meaning that the computers are located in different physical locations (i.e., aren't physically the same computer, e.g., are located in different countries, states, cities, rooms, etc.).

As used herein, the term “detection assay component” refers to a component of a system capable of performing a detection assay. Detection assay components include, but are not limited to, hybridization probes, buffers, and the like.

As used herein, the term “a detection assay configured for target detection” refers to a collection of assay components that are capable of producing a detectable signal when carried out using the target nucleic acid. For example, a detection assay that has empirically been demonstrated to detect a particular single nucleotide polymorphism is considered a detection assay configured for target detection.

As used herein, the phrase “unique detection assay” refers to a detection assay that has a different collection of detection assay components in relation to other detection assays located on the same detection panel. A unique assay doesn't necessarily detect a different target (e.g. SNP) than other assays on the same detection panel, but it does have a least one difference in the collection of components used to detect a given target (e.g. a unique detection assay may employ a probe sequences that is shorter or longer in length than other assays on the same detection panel).

As used herein, the term “candidate” refers to an assay or analyte, e.g., a nucleic acid, suspected of having a particular feature or property. A “candidate sequence” refers to a nucleic acid suspected of comprising a particular sequence, while a “candidate oligonucleotide” refers to an oligonucleotide suspected of having a property such as comprising a particular sequence, or having the capability to hybridize to a target nucleic acid or to perform in a detection assay. A “candidate detection assay” refers to a detection assay that is suspected of being a valid detection assay.

As used herein, the term “detection panel” refers to a substrate or device containing at least two unique candidate detection assays configured for target detection.

As used herein, the term “valid detection assay” refers to a detection assay that has been shown to accurately predict an association between the detection of a target and a phenotype (e.g. medical condition). Examples of valid detection assays include, but are not limited to, detection assays that, when a target is detected, accurately predict the phenotype medical 95%, 96%, 97%, 98%, 99%, 99.5%, 99.8%, or 99.9% of the time. Other examples of valid detection assays include, but are not limited to, detection assays that quality as and/or are marketed as Analyte-Specific Reagents (i.e. as defined by FDA regulations) or In-Vitro Diagnostics (i.e. approved by the FDA).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an as say, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining” to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc. “Allele frequency information” refers to facts or data pertaining allele frequencies, including, but not limited to, allele identities, statistical correlations between the presence of an allele and a characteristic of a subject (e.g., a human subject), the presence or absence of an allele in a individual or population, the percentage likelihood of an allele being present in an individual having one or more particular characteristics, etc.

As used herein, the term “assay validation information” refers to genomic information and/or allele frequency information resulting from processing of test result data (e.g. processing with the aid of a computer). Assay validation information may be used, for example, to identify a particular candidate detection assay as a valid detection assay.

As used herein, the term “coupled,” as in “coupled attachment,” refers to attachments between objects that do not, by themselves, provide a pressure-tight seal. For example, two metal plates that are attached by screws or pins may comprise a coupled attachment. While the two plates are attached, the seam between them does not form a pressure-tight seal (i.e., gas and/or liquid can escape through the seam).

As used herein, the term “synthesis and purge component” refers to a component of a synthesizer containing a cartridge for holding one or more synthesis columns attached to or connected to a drain plate for allowing waste or wash material from the synthesis columns to be directed to a waste disposal system.

As used herein, the term “cartridge” refers to a device for holding one or more synthesis columns. For example, cartridges can contain a plurality of openings (e.g., receiving holes) into which synthesis columns may be placed. “Rotary cartridges” refer to cartridges that, in operation, can rotate with respect to an axis, such that a synthesis column is moved from one location in a plane (a reagent dispensing location) to another location in the plane (a non-reagent dispensing location) following rotation of the cartridge.

As used herein, the term “nucleic acid synthesis column” or “synthesis column” refers to a container or chamber in which nucleic acid synthesis reactions are carried out. For example, synthesis columns include plastic cylindrical columns and pipette tip formats, containing openings at the top and bottom ends. The containers may contain or provide one or more matrices, solid supports, and/or synthesis reagents necessary to carry out chemical synthesis of nucleic acids. For example, in some embodiments of the present invention, synthesis columns contain a solid support matrix on which a growing nucleic acid molecule may be synthesized. Nucleic acid synthesis columns may be provided individually; alternatively, several synthesis columns may be provided together as a unit, e.g., in a strip or array, or as device such as a plate having a plurality of suitable chambers. Columns may be constructed of any material or combination of materials that do not adversely affect (e.g., chemically) the synthesis reaction or the use of the synthesized product. For example, columns or chambers may comprise polymers such as polypropylene, fluoropolymers such as TEFLON, metals and other materials that are substantially inert to synthesis reaction conditions, such as stainless steel, gold, silicon and glass. In some embodiments, chambers comprise a coating of such a suitable material over a structure comprising a different material.

As used herein, the term “seal” refers to any means for preventing the flow of gas or liquid through an opening. For example, a seal may be formed between two contacted materials using grease, o-rings, gaskets, and the like. In some embodiments, one or both of the contacted materials comprises an integral seal, such as, e.g., a ridge, a lip or another feature configured to provide a seal between said contacted materials. An “airtight seal” or “pressure tight seal” is a seal that prevents detectable amounts of air from passing through an opening. A “substantially airtight” seal is a seal that prevents all but negligible amounts of air from passing through an opening. Negligible amounts of air are amounts that are tolerated by the particular system, such that desired system function is not compromised. For example, a seal in a nucleic acid synthesizer is considered substantially airtight if it prevents gas leaks in a reaction chamber, such that the gas pressure in the reaction chamber is sufficient to purge liquid in synthesis columns contained in the reaction chamber following a synthesis reaction. If gas pressure is depleted by a leak such that synthesis columns are not purged (e.g., resulting in overflow during subsequent synthesis rounds), then the seal is not a substantially airtight seal. A substantially airtight seal can be detected empirically by carrying out synthesis and checking for failures (e.g., column overflows) during one or a series of reactions.

As used herein, the term “sealed contact point” refers to sealed seams between two or more objects. Seals on sealed contact points can be of any type that prevent the flow of gas or liquid through an opening. For example, seals can sit on the surface of a seam (e.g., a face seal) or can be placed within a seam, such that a circumferential contact is created within the seam.

As used herein, the term “alignment detector” refers to any means for detecting the position of an object with respect to another object or with respect to the detector. For example, alignment detectors may detect the alignment of a dispensing end of a dispensing device (e.g., a reagent tube, a waste tube, etc.) to a receiving device (e.g., a synthesis column, a waste valve, etc.). Alignment detectors may also detect the tilt angle of an object (e.g., the angle of a plane of an object with respect to a reference plane). For example, the tilt angle of a plate mounted on a shaft may be detected to ensure a proper perpendicular relationship between the plate and the shaft. Alignment detectors include, but are not limited to, motion sensors, infra-red or LED-based detectors, and the like.

As used herein, the term “alignment markers” refers to reference points on an object that allow the object to be aligned to one or more other objects. Alignment markers include pictorial markings (e.g., arrows, dots, etc.) and reflective markings, as well as pins, raised surfaces, holes, magnets, and the like.

As used herein, the term “motor connector” refers to any type of connection between a motor and another object. For example a motor designed to rotate another object may be connected to the object through a metal shaft, such that the rotation of the shaft, rotates the object. The metal shaft would be considered a motor connector.

As used herein, the term “packing material” refers to material placed in a passageway (e.g., a synthesis column) in a manner such that it provides resistance against a pressure differential between the two ends of the passageway (i.e. hinders the discharge of the pressure differential). Packing material may comprise a single material or multiple materials. For example, in some embodiments of the present invention, packing material comprising a nucleic acid synthesis matrix (e.g., a solid support for nucleic acid synthesis such as controlled pore glass, polystyrene, etc.) and/or one or more frits are used in synthesis columns to maintain a pressure differential between the two ends of the synthesis column. Packing material may be distributed into the reaction chambers in a variety of forms. For example, synthesis support matrix may be provided as a granular powder. In some embodiments, support matrix may be provided in a “pill” form, wherein an appropriate amount of a support material is held together with a binder to form a pill, and wherein one or more pills are provided to a reaction chamber, as appropriate for the scale of the intended reaction, and further wherein the binder is removed or inactivated (e.g., during a wash step) to allow the powdered matrix to function in the same manner as an unbound powder. The use of a pill embodiment provides the advantages of facilitating the process of pre-measuring synthesis support materials, allowing easy storage of support matrices in a pre-measured form, and simplifying provision of measured amounts of synthesis support matrix to a reaction chamber.

As used herein, the term “idle,” in reference to a synthesis column, refers to columns that do not take part in a particular synthesis reaction step of a nucleic acid synthesizer. Idle synthesis columns include, but are not limited to, columns in which no synthesis occurs at all, as well as columns in which synthesis has been completed (e.g., for short oligonucleotide) while other columns are actively undergoing additional synthesis steps (e.g., for longer oligonucleotides).

As used herein, the term “active,” in reference to a synthesis column, refers to columns that take part (or are taking part) in a particular synthesis reaction step of a nucleic acid synthesizer. Active synthesis columns include, but are not limited to, columns in which liquid reagents are being dispensed into, or columns that contain liquid reagents (e.g. waiting to be purged), or columns that are in the process of being purged.

As used herein, the term “O-ring” refers to a component having a circular or oval opening to accommodate and provide a seal around another component having a circular or oval external cross-section. An O-ring will generally be composed of material suitable for providing a seal, e.g., a resilient air-or moisture-proof material. In some embodiments, an O-ring may be a circular opening in a larger gasket. A single gasket may contain multiple openings and thus provide multiple O-rings. In other embodiments, an O-ring may be ring-shaped, i.e., it may have circular interior and exterior surfaces that are essentially concentric.

As used herein, the term “viewing window” refers to any transparent component configured to allow visual inspection of an item or material through the window. An enclosure may include a transparent portion that provides a viewing window for item within the disclosure. Likewise, an enclosure may be made entirely of a transparent material. In such embodiments, the entire enclosure can be considered a viewing window. A “viewing window” in an enclosure that is “configured to allow visual inspection” of items in the enclosure “without opening the enclosure” refers to a viewing window in an enclosure of sufficient size, location, and transparency to allow the item to be viewed, unhindered, by the human eye. For example, where the item is one or more reagent bottles, the window is configured to allow viewing of the reagents bottles by the human eye to determine if the bottles or full or empty. A window that does not provide adequate visual inspection of each of the reagent bottles is not configured to allow visual inspection of reagents in the enclosure without opening the enclosure.

As used herein, the term “enclosure” refers to a container that separates materials contained in the enclosure from the ambient environment (e.g., as in a sealed system). For example, an enclosure may be used with a reagent station to contain reagents within an interior chamber of the enclosure, and therefore separate the reagents from the ambient environment. In some embodiments, the enclosure provides an airtight or substantially airtight seal between the interior and exterior of the enclosure. The enclosure may contain one or more valves (e.g., ventilation ports), doors, or other means for allowing gasses or other materials (e.g., reagent bottles) to enter or leave the interior environment of the enclosure.

As used herein, the term “reaction enclosure” refers to an enclosure that separates the reaction columns or other reaction vessels (e.g., microplates) from the ambient environment. For example, a chamber bowl 18 closed with a top cover 30 and sealed with a chamber seal 31 is one exemplary embodiment of a reaction enclosure. Another example of a reaction enclosure is a synthesis case, e.g., as provided with a POLYPLEX synthesizer (GeneMachines, San Carlos, Calif.) and with the synthesizers described in WO 00/56445. In preferred embodiments, reaction enclosures can be sealed during at least one step of operation (e.g., during active synthesis) and can be opened for at least one step of operation (e.g., for inserting or removing reaction vessels).

As used herein, the term “top enclosure” refers to an enclosure that forms a primarily enclosed space over the top cover. In preferred embodiments, the top enclosure has four sides (e.g., four top enclosure sides, e.g., 98) and a top panel (e.g., 97) that form a primarily enclosed space (e.g. 104) above the top cover (e.g., 30) containing a plurality of valves (e.g., 10) and a plurality of dispense lines (e.g., 6). In some embodiments, the primarily enclosed space (e.g., 104) is open to the ambient environment through a ventilation slot (e.g., 100) in the top cover or the top enclosure. In certain embodiments, the top panel (e.g., 99) contains an outer window (e.g., 101).

Also as used herein, the combination of a “top enclosure” and “top cover” (e.g., formed as one unit, or connected together) is referred to collectively as the “lid enclosure”. In preferred embodiments, the “lid enclosure” (e.g., 102) has six sides, with the top cover (e.g., 30) serving as the “bottom”, the top panel serving as the surface opposite the top cover, and the four side walls being the top enclosure sides (e.g., 98). In certain embodiments, the lid enclosure is hinged so that is may be moved upward and downward.

As used herein, the term “primarily enclosed space” refers to a space having reduced contact with the ambient environment. A primarily enclosed space need not be sealed. For example, in some embodiments, a primarily enclosed space 104 of a lid enclosure of the present invention has contact with the ambient environment through a ventilation slot (e.g., 100). In some embodiments, a primarily enclosed space 104 of a synthesizer base 2 has contact with the ambient environment through a ventilation slot (e.g., 100) As used herein, the term “ventilated workspace” refers to a work area that is open to the ambient environment but that is maintained under negative air pressure such that air flows into the ventilated workspace, thereby reducing or preventing the flow of fumes and emissions from the ventilated workspace into the ambient environment. One example of a ventilated workspace is a fume hood (e.g. a chemical fume hood). In some embodiments, the ventilated workspace that is part of an apparatus (e.g., a nucleic acid synthesizer), such that the negative air pressure is maintained over a reaction chamber to draw air away from the reaction chamber so as to prevent the air from entering the ambient environment.

As used herein, the term “synthesis” refers to the assembly of polymers from smaller units, such as monomers.

As used herein, the term “fluidic connection” refers to a continuous fluid path between components.

As used herein, the term “parallel” refers to systems or actions functioning in an essentially simultaneous, side-by-side, manner (e.g., parallel synthesis or parallel synthesis system).

As used herein, the term “reaction support” refers to a structure supporting, comprising, or containing one or more reaction chambers.

As used herein, the term “rare mutation” refers to a mutation that is present in 20% or less (preferably 10% or less, more preferably 5% or less, and more preferably 1% or less) of a population of nucleic acid molecules in a sample (i.e., wherein the remaining 80% or more of the nucleic acid molecules have a wild type sequence or a different mutation in the corresponding region of the nucleic acid molecules).

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the phrase “non-amplified oligonucleotide detection assay” refers to a detection assay configured to detect the presence or absence of a particular polymorphism (e.g., SNP, repeat sequence, etc.) in a target sequence (e.g. genomic DNA) that has not been amplified (e.g. by PCR), without creating copies of the target sequence. A “non-amplified oligonucloetide detection assay” may, for example, amplify a signal used to indicate the presence or absence of a particular polymorphism in a target sequence, so long as the target sequence is not copied.

### GENERAL DESCRIPTION OF THE INVENTION

The present invention relates to detection assay development, production, usage and optimization. In particular, the present invention provides systems and methods for acquiring and analyzing biological information. The present invention also provides detection assay production with improved oligonucleotide synthesis and processing systems. The present invention further provides systems that integrate biological information collection with detection assay production that allow for rapid development of commercial products, such as analyte specific reagents (ASRs) and in vitro diagnostics (IVDs).

For example, the present invention provides systems and methods for the use of genetic information in the generation of assays for detecting the genetic identity of samples, the production of assays, the use of assays for gathering genetic information of individuals and populations, and the storage, analysis, and use of the obtained information, including the use of information in selecting detection assays for research use, use in panels, use as ASRs, and use in clinical diagnostics (e.g., in vitro diagnostics).

In some preferred embodiments, the present invention provides systems and methods for analyzing available sequence information (e.g., publicly available sequence information and information obtained by the methods described herein) in the selection of informative DNA and RNA target sequences for detections and analysis of individuals and populations. The present invention also provides systems and methods for the design and production of detection assays directed to such target sequences. The present invention further provides systems and methods for the collection, storage and analysis of data derived from detection assays.

Importantly, the present invention provides integrated systems and methods that exploit the synergies of the above systems and methods to provide comprehensive solutions, allowing for large scale and informative analysis of sequences for identifying genotype/phenotype correlations, measuring differences in gene expression, identifying allele frequencies in populations, and typing individuals and populations for important (e.g., medically relevant) sequences. For example, in some embodiments, the present invention applies data obtained from detection assays to improve the selection of target sequences, design of improved assays, and selection of assays that are suitable for use on multi-analyte panels, as ASRs, and for clinical diagnostics.

A general overview of the systems of the present invention is provided in FIG. 1. The present invention provides detection assay development, production and optimization (See, section A below). For example, orders are received from customer (e.g. a target sequence is entered via a web interface), and the orders are processed (See, section A.I., “Target Sequence Selection), and Detection Assays are Designed (See Section A.III, below). The designed assays are produced (or filled from inventory) in a production facility (See, section III below). The assays that are produced are stored in inventory or shipped to customers. Preferably, each of these components are operably linked to a central data management system (e.g. running enterprise software such as Oracle), such that data and status of orders is communicated throughout the system (See, Section A.IV., below).

Detection assays are shipped to customers who use the detection assay and generate data. In certain embodiments, the data generated by the use of these detection assays is gathered, analyzed, and stored (See, section A.V, below). This information may then be integrated with the order, design, production and storage components mentioned above (See, A.VI. below). In this regard, data is continuously generated that allows, for example, an association between detection assays or targets with particular medical conditions to be established.

Gathering, analyzing, and producing detection assays while generating association data allows the clinical detection assays (e.g., ASRs and In vitro Diagnostics) to be developed and validated (See, Section, B below) through a funneling process that allows a business to focus on particularly useful assays. Assays may be incorporated in panels or databases in order to be distributed to research facilities (e.g. ASR certified), hospitals, doctors, and other customers (See, Section, C below). Employing these detection assays, or panels of assays, in a clinical setting, for example, further allows data to be collected and further associated with a patient's medical records (e.g. See, D, below). This increases the value of data that is collected and shared with the management systems of the present invention. Integrating the production systems, databases, and managements systems of the present invention allows efficient production of particular assays, as well as rapid identification of ASRs, and in vitro diagnostics. Furthermore, integration of these systems allows for accurate business pricing of various assays (See, section C, below), allowing, for example, differential pricing of ASRs and In Vitro Diagnostics.

### DETAILED DESCRIPTION OF THE INVENTION

The following discussion provides a description of certain preferred illustrative embodiments of the present invention and is not intended to limit the scope of the present invention. For convenience, the discussion focuses on the application of the present invention to the detection of DNA targets, but it should be understood that the methods and systems are intended for use in the development of tools for the analysis of any nucleic acid analyte, e.g., DNA or RNA. Also, for the sake of illustration, the discussion often focuses on the characterization of SNPs using INVADER assay technology. It should be understood that the methods and systems of the present invention are intended for use in detecting other biologically relevant factors using a wide variety of detection assay technologies.

As discussed above, the present invention provides systems and methods for developing detection assays for research and clinical use. The following sections describe the high throughput design, optimization, and production of detection assays in a manner that allows assays to pass from a discovery phase to use as clinical diagnostic assays. The description is provided in the following sections: A) Detection Assay Development, Production, and Optimization; B) Development of Clinical Detection Assays; C) Distribution and Use of Detection Assays, D) Medical Records; and E) Financial Component.

A. Detection Assay Development, Production, and Optimization The detection assay development, production, and optimization is illustrated below for hybridization-bases assays. One skilled in the art will appreciate the general applicability of various aspects of this description to other types of detection assays. The discussion of detection assay development, production, and optimization is provided in the following sections: I) Target Sequence Selection; II) Detection Assay Design; III) Detection Assay Production; IV) Data Management Systems; V) Detection Assay Use and Data Generation and Collection; and VI) Integrated Information, Design, and Production (Optimization). It will be appreciated that every step may not be required for each detection assay. For example, where a valid target sequence and assay design are already known, production and testing may be started directly. The steps may be used for original assay development and/or may be used to re-evaluate a pre-existing detection assay, whether is be for a research or a clinical detection assay. Examples of process configurations for integrating the steps (e.g., with software) are provided in FIGS. 1, 58, 61, and 62. As shown in FIG. 1, direct clients or distributors go through an order entry process (described in detail below). Detections assays corresponding to particular oligonucleotides, primers, panels, polymorphisms (e.g., SNPs) are entered and process through an in silico validation process (described in detail below) and assay design software (e.g., INVADERCREATOR software). If a request corresponds to a previously validated or ordered sequences, software locates the product and proceeds with the order accordingly. Designed detection assays are then sent to a production facility for production and validation (described in detail below). Data generated by the process or from use of the detection assays and collected and stored in databases (described in detail below).

I. Target Sequence Selection

The ability to detect the presence or absence of specific target sequences in a sample underlies much of the fields of molecular diagnostics and molecular medicine. For example, tremendous effort has been expended in the development of detection assays for nucleic acid sequence mutations that correlate to phenotypes of interest (e.g., inherited diseases). During the development of the present invention, it was found that the design of a detection assay based on a published target sequence was often not sufficient to produce viable assays. In some circumstances assays will not work at all. In others, they may work for particular individuals or populations, but fail with other individuals or populations. The present invention provides * systems and methods for selecting appropriate target sequences that can be successfully targeted by detection assays.

The problem with existing methods and the solutions provided by the present invention can be illustrated by example. Many detection assays are based on the principle of nucleic acid hybridization. An oligonucleotide is designed to hybridize to a portion of the target sequence; the presence of the hybrid, or the cleavage, elongation, ligation, disassociation, or other alterations of the oligonucleotide are detected as a means for characterizing the presence or absence of the sequence of interest (e.g., a SNP). Because there is sequence heterogeneity in the population, an oligonucleotide designed to hybridize to a target sequence of one individual may not hybridize to the corresponding sequence from another individual. For example, a first individual may have a gene sequence containing a SNP that is to be detected. A second individual may have the SNP, but also may have additional sequence differences in the vicinity of the SNP that prevent the hybridization of an oligonucleotide that was designed based on the sequence of the first individual. Additionally, target sequence information obtained from a public source may contain errors (e.g., may provide the wrong sequence) or may comprise incomplete, but essential, information. For example, a given target sequence may be found in multiple locations in the genome-the intended region that the assay is designed to detect, and unintended regions that would result in false positive or otherwise misleading assay results.

The systems and methods of the present invention provide an analysis of candidate target sequences to determine if they are suitable for use in detection assays. The systems and methods of the present invention also select appropriate sequences that are likely to function in the intended detection assay. This aspect of the present invention is referred to herein as “in silico analysis,” as computer analysis is conducted to analyze candidate target sequences against sequence and sequence-related information databases. In silico analysis may be performed prior to, or in conjunction with other processes of the present invention (e.g., detection assay design and production, selection of materials for panels, ASRs, and clinical tests, etc.).

In silico analysis methods of the present invention include one or more of the following sequence analysis and processing steps: input of a candidate sequence; editing of the candidate sequence, where necessary; screening of the candidate sequence for repeat sequences; screening of the candidate sequence for research artifact sequences; identification of the candidate sequence in a sequence database; conformation of the candidate sequence in a second (or additional) sequence database; information gathering using one or more sequence information databases; problem reporting; and/or transmission of an approved target sequence for production (e.g., automated production).

A. Sequence Input (Order Entry Component)

Sequences may be input for in silico analysis from any number of sources. In many embodiments, sequence information is entered into a computer. The computer need not be the same computer system that carries out in silico analysis. In some preferred embodiments, candidate target sequences may be entered into a computer linked to a communication network (e.g., a local area network, Internet or Intranet). In such embodiments, users anywhere in the world with access to a communication network may enter candidate sequences at their own locale. In some embodiments, a user interface is provided to the user over a communication network (e.g., a World Wide Web-based user interface), containing entry fields for the information required by the in silico analysis (e.g., the sequence of the candidate target sequence).

The use of a Web based user interface has several advantages. For example, by providing an entry wizard, the user interface can ensure that the user inputs the requisite amount of information in the correct format. In some embodiments, the user interface requires that the sequence information for a target sequence be of a minimum length (e.g., 20 or more, 50 or more, 100 or more nucleotides) and be in a single format (e.g., FASTA). In other embodiments, the information can be input in any format and the systems and methods of the present invention edit or alter the input information into a suitable form for in silico analysis. For example, if an input target sequence is too short, the systems and methods of the present invention search public databases for the short sequence, and if a unique sequence is identified, convert the short sequence into a suitably long sequence by adding nucleotides on one or both of the ends of the input target sequence. Likewise, if sequence information is entered in an undesirable format or contains extraneous, non-sequence characters, the sequence can be modified to a standard format (e.g., FASTA) prior to further in silico analysis. The user interface may also collect information about the user, including, but not limited to, the name and address of the user. In some embodiments, target sequence entries are associated with a user identification code.

In certain embodiments, there is a separate component for entering large orders (e.g. entered by large companies), a separate component for entering small orders (e.g. entered by individual researchers), and a separate component for clinical orders (e.g. hospitals and clinical laboratories). In some embodiments, sequences are input directly from assay design software (e.g., the INVADERCREATOR software described below).

In preferred embodiments, each sequence is given an ID number. The ID number is linked to the target sequence being analyzed to avoid duplicate analyses. For example, if the in silico analysis determines that a target sequence corresponding to the input sequence has already been analyzed, the user is informed and given the option of by-passing in silico analysis and simply receiving previously obtained results.

The customer order component also includes one or more screens or web pages that include detection assay instrumentation data. Detection assay instrumentation data includes data describing various systems and devices, including but not limited to liquid handlers, workstations, and other automation options shown in, for example, Table 2, which are used to facilitate use of the detection assays created using the methods and systems described herein. By way of example, once a customer selects a particular type of panel format, e.g. 96 well, 385 well or 1536 well and assay configuration, he is automatically linked or presented with data of appropriate corresponding devices that are used to read the panel format which are offered for sale to the customer. In another variant, the system stores information about the type of instrumentation the customer already has in house or has previously purchased, and automatically determines and suggests the type of panel format for detection assays that the customer should buy on the customer order component, e.g. 96 well, 384 well or 1536 well. By way of further example, the customer is also provided with instrumentation pricing data, instrument specification data, delivery data, shipping data, for various combinations of instrumentation that would suit the customer's needs. The customer order entry component can then feed data on the customer's instrumentation order (or in-house instrumentation where the customer makes a selection from an instrumentation menu presented on the web site) to the detection assay production component (including resident hardware and software components thereof) so that projections can be made as to the number and type of various detection assay starting materials that need to be purchased or stocked based upon the customers selection of instrumentation and projected usage of disposable detection assays, e.g. reagents, glass slides, plastic arrays, etc.

In yet a further embodiment, a single customer's (or a plurality of networked customers') instrumentation has a communication link to the customer order component or the detection assay production facility for exchanging data therebetween. It is appreciated that detection assay usage data is transferred from the customer's instrumentation to the detection assay production facility (or other components of the system) to help schedule and produce detection assays and order reagents and components therefore, or prompt the customer via e-mail that his stock of detection assays is nearing a predetermined number and that the customer needs to re-order detection assays. In another variant, once a threshold usage number of detection assays is determined, the customer's instrumentation automatically sends order data to the customer order component or other component of the system automatically ordering additional detection assays for one or more customers. In some embodiments, these systems are linked to a pricing component, wherein repeat customers may receive beneficial pricing for re-orders or upon reaching a total threshold volume of orders over time.

B. Web-Ordering Systems and Methods

Users who wish to order detection assays, have detection assay designed, or gain access to databases or other information of the present invention may employ an electronic communication system (e.g., the Internet). In some embodiments, an ordering and information system of the present invention is connected to a public network to allow any user access to the information. In some embodiments, private electronic communication networks are provided. For example, where a customer or user is a repeat customer (e.g., a distributor or large diagnostic laboratory), the full-time dedicated private connection may be provided between a computer system of the customer and a computer system of the systems of the present invention. The system may be arranged to minimize human interaction. For example, in some embodiments, inventory control software is used to monitor the number and type of detection assays in possession of the customer. A query is sent at defined intervals to determine if the customer has the appropriate number and type of detection assay, and if shortages are detected, instructions are sent to design, produce, and/or deliver additional assays to the customer. In some embodiments, the system also monitors inventory levels of the seller and in preferred embodiments, is integrated with production systems to manage production capacity and timing.

In some embodiments, a user-friendly interface is provided to facilitate selection and ordering of detection assays. Because of the hundreds of thousands of detection assays available and/or polymorphisms that the user may wish to interrogate, the user-friendly interface allows navigation through the complex set of options. For example, in some embodiments, a series of stacked databases are used to guide users to the desired products. In some embodiments, the first layer provides a display of all of the chromosomes of an organism. The user selects the chromosome or chromosomes of interest. Selection of the chromosome provides a more detailed map of the chromosome, indicating banding regions on the chromosome. Selection of the desired band leads to a map showing gene locations. One or more additional layers of detail provide base positions of polymorphisms, gene names, genome database identification tags, annotations, regions of the chromosome with pre-existing developed detection assays that are available for purchase, regions where no pre-existing developed assays exist but that are available for design and production, etc. (See, FIGS. 2a-f). Selecting a region, polymorphism, or detection assay takes the user to an ordering interface, where information is collected to initiate detection assay design and/or ordering. In some embodiments, a search engine is provided, where a gene name, sequence range, polymorphism or other query is entered to more immediately direct the user to the appropriate layer of information.

In certain embodiments, a user may select a PCR (or other amplification technology) or non-PCR option, depending if they want to employ amplification along with their detection assay. The PCR primer section may be employed to design such assays, taking into consideration the target and the detection assay selected by the user (see below).

In some embodiments, the ordering, design, and production systems are integrated with a finance system, where the pricing of the detection assay is determined by one or more factors: whether or not design is required, cost of goods based on the components in the detection assay, special discounts for certain customers, discounts for bulk orders, discounts for re-orders, price increases where the product is covered by intellectual property or contractual payment obligations to third parties, and price selection based on usage. For example, where detection assays are to be used for or are certified for clinical diagnostics rather than research applications, pricing is increased. In some embodiments, the pricing increase for clinical products occurs automatically. For example, in some embodiments, the systems of the present invention are linked to FDA, public publication, or other databases to determine if a product has been certified for clinical diagnostic or ASR use.

In one variant of the invention, the system and method of the present invention includes an organism-specific web order entry component. The organism-specific web order entry component comprises one or more screens and/or linked web pages that are interactively directed to present for sale one or more detection assays for a specific organism(s). By way of example, a web page or combination of web pages provides displays of the chromosomes, genes, and/or detection assays for various transgenic plants, wild type plants, wild type animals, transgenic animals, and/or genetically altered or naturally occurring microorganisms, e.g. bacteria, viruses, etc. By way of further example, one or more screens of different linked web pages permit a user to drill down into a specific genus, species and/or sub-species of an organism and/or chromosomes (or sub-parts thereof), and display the various detection assays created for the organism and/or detection assays that have been created that may be used across various organisms. The detection assays are optionally linked to specific genes or portions of chromosomes of a single organism or of multiple related or unrelated organisms.

C. In Silico Processing Systems

In silico analysis utilizes one or more sequence and information databases (e.g., public or private sequence databases) and software applications for processing sequence and database information (See, e.g. FIG. 3). In some preferred embodiments, databases and software for in silico analysis are housed in a single location on one or more computers. Housing the databases and processing software locally provides increased and consistent speed and access to information. In other embodiments, one or more databases and software components located on external computers are accessed over a communication network (e.g., accessed over the World Wide Web).

In preferred embodiments, databases that are maintained locally are updated regularly (e.g., following each update of the web-based server, a new version is downloaded to local servers). In some preferred embodiments, databases are surveyed periodically to determine if a new version is available and, if so, one is downloaded. In some preferred embodiments, more than one copy of each database is available locally. In particularly preferred embodiments, downloaded data is parsed to extract the data, and the parsed data is configured to automatically populate the fields of one or more receiving databases (e.g., an association database, a SNP database). In some embodiments, Perl scripts are used to sort data, e.g., line-by-line, and to create new text files (e.g., having data tagged according to the receiving field in the receiving database) for importation into the fields of a receiving database.

In some embodiments, the database analysis system comprises one or more central nodes (e.g., a computer containing a processor and computer memory) and a plurality of sub-nodes. In some embodiments, the sub-nodes house individual databases (or portions thereof) or software programs. In preferred embodiments, the central node controls the flow of information between sub-nodes, sending search requests to the sub-nodes and receiving search results from the sub-nodes. For example, in some embodiments, the central node directs data (e.g., candidate target sequence) to a sub node for a database search, receives the results, and directs the information to another sub-node for additional database searching. In some preferred embodiments, the central node directs information to multiple sub nodes simultaneously (e.g., for multiple concurrent database searches).

In some embodiments, in order to increase database access speed, individual databases are split among multiple (e.g., two) sub-nodes. In other embodiments, databases are housed on a single node. In preferred embodiments, databases are present in multiple copies on multiple sub-nodes. In some preferred embodiments, the central node monitors database load and status on each sub-node and directs searches to the node with the greatest available capacity.

In some preferred embodiments, the central node further directs resource management software. For example, individual nodes are sent test sequences on a regular basis to ensure that they are receiving information and processing information on a desired time scale. If a sub node is found to not be functioning properly, the central node directs information to a secondary sub node containing a copy of the database. In other embodiments, sub-nodes conduct self-monitoring routines and send status reports back to the central node. For example, in some embodiments, if a search on a sub-node fails or times out, the sub-node reports this information back to the central node so that appropriate action can be taken (e.g., send the search to another node and/or flag a particular sub-node for intervention). In some preferred embodiments, the central node maintains a queue of jobs submitted to each sub-node and warns human supervisors if a job fails to be completed.

In some embodiments, the central node comprises one or more workstations. In some embodiments, the sub nodes comprise two or more workstations. In other embodiments, the sub nodes comprise 5 or more workstations. In yet other embodiments, the sub nodes comprise 10 or more workstations. The present invention is not limited to a particular model or type of workstation. One skilled in the art understands that a variety of new processors of increasing speeds are regularly introduced into the market and that any suitable work station may be substituted for those described herein.

In some embodiments, in silico analysis of a candidate target sequence is completed in less than 10 seconds. In some preferred embodiments, in silico analysis of a candidate target sequence is completed in less than 2 seconds. In still more preferred embodiments, in silico analysis is completed in less than one second. In some embodiments, more than one (e.g., at least 5, preferably at least 20, and even more preferably, at least 100) sequences are analyzed simultaneously using the in silico analysis system of the present invention.

1. Preliminary Sequence Screening

In some embodiments of the present invention, the first step of in silico analysis of candidate target sequences is prescreening the candidate target sequences to maximize sequence database search efficiency.

In some embodiments, candidate target sequences are searched for repeat sequences. “Repeat sequences” refers to sequences that are known to repeat multiple times in a sample (e.g., in an organism's genome). Many genomes contain large regions of repeated sequences. The presence of repeated sequences in detection assay hybridization oligonucleotides can cause the oligonucleotide to hybridize to sequences other than, and/or in addition to, the intended target. Additionally, because repeat sequences are found in multiple copies in the genome, databases searches may operate very slowly or may not proceed. In some embodiments, RepeatMasker is a perl script used in conjunction with REPBASE, which is a database of known Human repeats and is used to screen for repeat sequences. Repeat Masker screens DNA sequences for interspersed repeats and low complexity DNA sequences. Sequence information in FASTA format is input through a web-browser interface or by uploading a file. Multiple sequences may be input at once or may be contained within a file. There is no limit to the length of the query sequence or size of the batch file. Sequence comparisons in RepeatMasker are performed by the program Cross-match, an implementation of the Smith-Waterman-Gotoh algorithm developed by Phil Green. In some embodiments, RepeatMasker is run using MaskerAid (Bioinformatics 16:1040-1 [2000], available through licensing from Washington University in Saint Louis, Mo.), a performance enhancer for RepeatMasker. Execution profiling of native RepeatMasker showed that the vast majority of its time was spent running Cross-Match. MaskerAid allows the faster WU-BLAST search engine to substitute transparently for CrossMatch, yielding speed improvement while effectively maintaining sensitivity. MaskerAid is fundamentally a software “wrapper” around WU-BLAST that makes it appear and function very much like CrossMatch.

The output of the program is an annotation of the repeats that are present in the sequence of interest as well as a modified, version of the sequence in which all the annotated repeats have been masked. The program returns three or four output files for each query. One contains the submitted sequence(s) in which all recognized interspersed or simple repeats have been masked. In the masked areas, each base is replaced with an N, so that the returned sequence is of the same length as the original. A table annotating the masked sequences as well as a table summarizing the repeat content of the query sequence is returned. Optionally, a file with alignments of the query with the matching repeats is returned as well.

Regions of low complexity, like simple tandem repeats, polypurine and AT-rich regions can lead to spurious matches in database searches. By default they are masked along with the interspersed repeats. With the option “Do not mask simple . . . ” only interspersed repeats are masked. This may, for example, be preferred in some embodiments where the masked sequence will be analyzed by a gene prediction program. Alternatively, with the option “Only mask simple . . . ”, one can mask only the low complexity regions (e.g., in some embodiments in which it is desirable to quickly locate polymorphic simple repeats in a sequence).

When checked, the repeat sequences are replaced by Xs instead of Ns. This allows one to distinguish the masked areas from possibly existing ambiguous sequences or other stretches of Ns in the original sequence. In some embodiments the use of X, N, or both may be desired for compatibility with database search engines used in the subsequent steps of the in silico analysis. In some embodiments, only the masked candidate target sequence is used in further in silico analysis. In other embodiments, both the masked and unmasked sequences are used in subsequent searches.

In certain cases, a majority or the entirety of the candidate target sequence may be masked by RepeatMasker. When this occurs, in some embodiments, a warning is sent to the user indicating that a potentially undesirable amount of the target sequence comprises repeat sequence. The user is then give the option of selecting a different target sequence or proceeding with the original sequence (or electing both options). When a decision to proceed with the sequence is selected, an unmasked version of the sequence is processed through the remaining in silico analysis steps. Where there is a portion of the original candidate target sequence that is not masked, both unmasked and masked sequences may be processed through the remaining in silico analysis steps. In some embodiments, in silico analysis is discontinued and the candidate target sequence is sent to production (Section III, below).

In some embodiments, prior to screening for repeat sequences, an analysis is performed to determine if the candidate target sequence contains undesired artifact sequences. For example, a number of sequences deposited in public databases contain vector sequence or other sequence artifacts as a result of molecular biology handling during their initial isolation and characterization. These artifact sequences often represent synthetic sequences not corresponding to a genome sequence, or inappropriately corresponding to a genome sequence other than the intended target. Where candidate target sequences are selected that contain artifact sequences, they are more likely to fail in detection assays and are more likely to result in undesirably long search times during the remaining in silico analysis steps. For example, rather than representing a sequence that appears once in a human genome, artifact sequence may correspond to thousands of deposited database sequence that each mistakenly contain a common vector sequence.

To correct for artifact sequence, in some embodiments, the present invention employs VecScreen (available at the National Center for Biotechnology Information, National Library of Medicine, National Institutes of Health public web site). VecScreen provides a system for identifying segments of a nucleic acid sequence that may be of vector origin. VecScreen searches a query for segments that match any sequence in a specialized non-redundant vector database (UniVec). The search uses a BLAST search routine with parameters preset for optimal detection of vector contamination. Those segments of the query that match vector sequences are categorized according to the strength of the match, and their locations are displayed.

The sequence of any vector contamination should theoretically be identical to the known sequence of the vector. In practice, occasional differences are expected to arise from sequencing errors, and less frequently, from engineered variants or spontaneous mutations. The search parameters used for VecScreen are chosen to find sequence segments that are identical to known vector sequences or which deviate only slightly from the known sequence. Vector containing sequences identified are then masked.

In some embodiments, the Repeat Masker and VecScreen screening are combined into a single search. In preferred embodiments, the candidate target sequence is first screened by, VecScreen, with the results then passed through Repeat Masker. Once the screening is complete, masked sequences and/or unmasked sequences are ready for database searching as described below.

2. Database Searches

In some embodiments, database searches are performed on the candidate target sequences. Databases searches are used, among other purposes, to confirm that 1) the candidate target sequence is a sequence corresponding to a known sequence, 2) the candidate target sequence corresponds to a unique sequence in the, sample to be tested, and 3) the candidate target sequence corresponds to a reliable (e.g., confirmed) sequence. The database searches are also used to gather information (allele frequencies, disease associations, variants, location in a genome, associated patents and patent applications, etc.) about the candidate target sequence. In some embodiments, the output information from the database searches is stored in a file associated with the candidate target sequence. In further embodiments, the output information is displayed to the user.

The present invention is not limited to the databases disclosed herein. Any database that provides relevant information may find use in the searches of the present invention. In some embodiments, searches are performed consecutively. In other embodiments, searches are performed concurrently. In preferred embodiments, some searches are performed consecutively and others are performed concurrently. In some embodiments, searches are performed using BLAST (Basic Local Alignment Search Tool) search mode using FASTA formatted sequences. In preferred embodiments, results from database searches are output as text files. Results are then converted to a format that is suitable for import into an Oracle database. In some embodiments, the BioJava Project is used to convert text output into an XML-like stream that is then incorporated into an Oracle database.

Other databases that are searched or used in or with various components of the invention include rat, mouse or any other organism sequence databases. It is also appreciated that the present invention can cross reference detection assays across different species of organisms. By way of example, if a customer designates a human detection assay on a customer order entry screen, the software or routines of the invention may automatically present and offer for sale on the customer's computer screen the same or similar detection assay for rats, mice or any other organism.

Descriptions of several databases that are searched in preferred embodiments of the present invention are described below.

i. SNP Databases

In preferred embodiments, candidate target sequences are first used to search several databases which catalog SNPs. The targeted databases include NCBI's dbSNP, the UK's HGBASE SNP database, the SNP Consortium database, and the Japanese Millenium Project's SNP database. The dbSNP database serves as a central repository for both single base nucleotide substitutions and short deletion and insertion polymorphisms, and includes all the SNPs identified in the SNP Consortium effort, 10% of the Japanese SNP database and 50% of the HGBASE SNP database. The data in dbSNP is integrated with other NCBI genomic data. If a match is found in the dbSNP, the output from the search is a dbSNP accession number, which is then tied in silico to identification and characterization of genomic landscape features including known genes, predicted genes; functional location and physical location in the genome. Functional location specifies where the SNP falls within a gene or predicted gene, and details the location as exonic, promotor, intronic, 5′ and 3′ untranslated flanking region. The physcial location includes the base pair position of the SNP on the individual chromosome. The base pairs that make up a chromosome are counted from the p telomere to the q telomere, starting with the first base pair on the p telomere. The physical location also includes the cytoband designation that contains the SNP of interest. In some embodiments, the dbSNP search returns an accession # with an RS designation. This designation indicates that the SNP is a unique SNP identified as common between multiple studies. The RS designation is used to perform additional database mining to harvest information relating to allele frequencies, penetrance estimates and heterozyosity estimates.

ii. Gene Loci Analysis

In some embodiments, following dbSNP searches, gene loci databases (e.g., Locus Link) are searched. LocusLink provides a single query interface to curated sequence and descriptive information about genetic loci. It presents information on official nomenclature, aliases, sequence accessions, phenotypes, EC numbers, MIM numbers, UniGene clusters, homology, map locations, protein domains, and related web sites. The information output from LocusLink includes a LocusLink accession number (LocusID), an NCBI genomic contig number (NT#), a reference mRNA number (NM#), splice site variants of the reference mRNA (XM#), a reference protein number (NP#), an OMIM accession number, and a Unigene accession number (HS#).

iii. Disease Association Databases

Following the LocusLink search, the information returned is used to search disease association databases. In some embodiments, the HUGO Mutation Database Initiative, which contains a collection of links to SNP/mutation databases for specific diseases or genes, is searched.

In some embodiments, the OMIM database is searched. OMIM (Online Mendelian Inheritance in Man) is a catalog of human genes and genetic disorders developed for the World Wide Web by NCBI, the National Center for Biotechnology Information. The database contains textual information and references. Output from OMIM includes a modified accession number where multiple SNPs are associated with a genetic disorder. The number is annotated to designate the presence of multiple SNPs associated with the genetic disorder.

iv. Gene Oriented Cluster Analysis

In some embodiments, following dbSNP searches, software (e.g., including but not limited to, UniGene) is used to partition search results into gene-oriented clusters. UniGene is a system for automatically partitioning GenBank sequences into a non-redundant set of gene-oriented clusters. Each UniGene cluster contains sequences that represent a unique gene, as well as related information such as the tissue types in which the gene has been expressed and map location. In addition to sequences of well-characterized genes, hundreds of thousands novel expressed sequence tag (EST) sequences are included in UniGene. Currently, sequences from human, rat, mouse, zebrafish and cow have been processed.

Unigene can be searched using either the UniGene accession number identified using LocusLink (preferred if available) or can be BLAST searched using the SNP target sequence of interest in FASTA format.

V. SNP Consortium Database

In some embodiments, masked sequences are used to search the SNP Consortium (TSC) database (available at SNP Consortium Ltd public web site). In some embodiments, SNP Consortium searches are conducted concurrently with dbSNP, LocusLink, UniGene, and OMIM searches. The SNP Consortium database includes mapping and allele frequency information. The database is searched via BLAST using the masked input target sequence. The output from the SNP Consortium database includes a TSC accession number and a Goldenpath Contig accession number in addition to mapping and allele frequency information (if known).

vi. Genome Databases

In some embodiments, target sequences are used to search genome databases (e.g., including but not limited to the Golden Path Database at University of California at Santa Cruz (UCSC) and GenBank). The GoldenPath database is searched via BLAST using the sequence in FASTA format or using the RS# obtained from dbSNP. GenBank is searched via BLAST using the masked sequence in FASTA format. In some embodiments, GoldenPath and GenBank searches are performed concurrently with TSC and dbSNP searches. In some embodiments, the searches result in the identification of the corresponding gene. Output from GenBank includes a GenBank accession number. Output from both databases includes contig accession numbers.

In some embodiments, a match to an incomplete gene is identified. In these cases, the automated system of the present invention directs the search of databases of unfinished genomic sequences (e.g., including but not limited to The High Throughput Genomic (HTG) Sequences database, a database that includes unfinished sequences from DDBJ, EMBL, and GenBank). Unfinished HTG sequences containing contigs greater than 2 kb are assigned an accession number and deposited in the HTG division. A typical HTG record might consist of all the first pass sequence data generated from a single cosmid, BAC, YAC, or P1 clone that together comprise more than 2 kb and contain one or more gaps. A single accession number is assigned to this collection of sequences and each record includes a clear indication of the status (phase 1 or 2) plus a prominent warning that the sequence data is “unfinished” and may contain errors. The accession number does not change as sequence records are updated; only the most recent version of a HTG record remains in GenBank. ‘Finished’ HTG sequences (phase 3) retain the same accession number, but are moved into the relevant primary GenBank division.

If a gene is identified using an unfinished sequence database, the information is transferred to the Oracle database of the present invention. If a gene is not identified, the automated system periodically (e.g., weekly) searches the databases for such information.

vii. Private Databases

In some embodiments of the present invention, private databases are searched. For example, the present invention provides systems and methods for gathering, organizing, and storing sequence information (See e.g., Sections III, IV and V, below). Information obtained by the methods of the present invention may be searched during target sequence analysis to assist in the confirmation or selection of target sequences that are likely to be successful in the desired detection assay (e.g., information obtained from previously successful assays is used to select or predict successful sequences for subsequent assays on the same or similar targets using the same or similar types of detection assay).

viii. Patent Databases

In some embodiments of the present invention, patent databases are searched. In some embodiments, a search is conducted to identify patents and patent applications related to a target or probe sequence. For example, patent claims may relate to target sequences, target SNPs, probe sequences and methods of using these compositions. Searchable databases of patented sequences may be public or private. Examples of tools for searching for patented sequences include GENESEQ and The Patent Agent. GENESEQ (Derwent Information, Alexandria Va.) searches for patented sequences in basic patents from 40 patent issuing authorities worldwide. GENESEQ provides a flat file (ASCII) EMBL-based format to enable integration into bioinformatics systems. The Patent Agent (DoubleTwist, Inc., Oakland, Calif.) uses the BLAST2N and BLAST2P algorithms to search Derwent's GENESEQ patent database and GenBank's patent division for sequence patent records matching an input (query) sequence.

3. Processing of Database Information

The collection of information obtained from the database searches is analyzed and/or stored. In some embodiments, the candidate target sequence is identified as a “high probability” target sequences and the results are reported (e.g. via the world wide web) to a user (to recommend production or use) or the target is directly sent on for production (Section III, below) or used. A high probability target sequence is one where the target sequence was confirmed to exist in one or more sequence databases, where there is no identified disagreement between the sequence databases (e.g., disagreement relating to the sequence of the target, the location of the target, or the presence of known mutations within the target region), where the target sequence represents a unique sequence in the samples that are to be assayed, and where the sequence corresponding to the target is considered reliable (i.e., confirmed or completed) sequence. In some embodiments, where a report is sent to a user, the report may include results of each search, a summary of the results, a general indication that the target sequence is a high probability sequences, and/or any other detailed information identified by the searches (e.g., disease association information).

In some embodiments of the present invention, where one or more problems are identified with the candidate target sequence, a report is sent (e.g. by the internet) to a user (e.g., the person who input or requested the candidate target sequence or a technician utilizing the systems and methods of the present invention) highlighting the one or more problems. Problems include the presence of repeat or artifact sequences in the candidate target sequences, multiple copies of the target sequence in the sample to be assayed (e.g., in the human genome), absence of the sequence in one or more of the databases, inconsistent results from one or more the databases (e.g., inconsistency as to the sequence corresponding to the target, the location of the target within a genome, the presence or location of a mutation or SNP to be assayed, and the presence or absence of one or more additional mutations or SNPs within the target region), and/or the sequence quality (reliability) of the sequence from the databases. In some embodiments, a reliability score is generated based on the presence or absence of one or more of the above potential problems. The reliability score may be sent to the user, or may be used as a signal to cause a further action, such as to begin production and/or to cancel the candidate target sequence.

In some embodiments, the user is given the option to select another target sequence or to proceed with the present target sequence (e.g., to proceed to production). In some embodiments, when problems are identified, the systems of the present invention automatically select and test additional candidate target sequences based on the original requested candidate target sequence (e.g., select neighboring sequences and/or remove problem portions of the sequence). If more reliable sequences are identified, these suggested alternate target sequences are reported to the user.

An overview of in silico analysis in some preferred embodiments of the present invention is shown in FIG. 3. The three top boxes represent exemplary sources of target sequences: research & development (e.g., direct input by research personnel) (20), Web interface (sequence input through a communication network) (21), and system administrators (e.g., to test the systems and methods of the present invention) (22). The target sequences are then analyzed by a screening component (23) that masks repeat and artifact sequences. If sequences are suitable for further analysis, they are passed to a series of databases. In the example shown in FIG. 3, the sequences are simultaneously sent to dbSNP (24), GoldenPath (25), and SNP Consortium (26) databases. If a dbSNP accession number is available, dbSNP data (27) is collected and stored and the dbSNP accession number is used to search the Unigene database (29). The dbSNP accession number may also be used to search the OMIM database (28) (which may also be searched after any other database search). If a dbSNP accession is not identified, the target sequence information is passed to the Unigene database (29). If a Unigene identification is found, Unigene data (30) is collected and stored.

The target sequence information sent to the GoldenPath database, (25) is used to identify the base pair position of the SNP on the current GoldenPath assembly of the genome and to check the reliability status of the sequence. If the sequence is considered “finished” sequence, GoldenPath data is collected and stored. If the sequence is not finished, the GenBank database (31) is searched to identify a GenBank contig identification number and to determine if the contig is considered “finished.” If the contig is finished, data is collected and stored. If the contig is not considered finished, a request for additional sequence data is placed with the group responsible with finishing the sequence of the region (32). If sequence data is available, data from the finishing group is collected and stored. The base pair position of the SNP generates the next level of in silico analysis to generate the genomic landscape information for each SNP resulting in a detailed in silico annotation of the SNP. The annotation is extended to include the full target sequence information. Target sequences which fall within a known gene region defined as “genic” to include 10 kilobases of sequence 5′ and 3′ of the beginning and end of transcription, then a second round of in silico annotation charaterizes this genic region as well.

The target sequence information sent to the SNP Consortium database (26) is used to identify a TSC identification number and TSC data, if available, is collected and stored. In some embodiments, one or more database accession numbers (e.g., LocusLink accession number) are provided during the original target sequence input or at any time thereafter, and said accession numbers are used to direct searches in the corresponding database (e.g., LocusLink database) or other databases. To the extent that databases searches are conducted solely to obtain an accession number for use in searching other databases, pre-entry of the accession number reduced the time required for in silico analysis. All of the collected data is stored in a database and used to generate reports and/or reliability scores for use in determining whether production of an assay directed at the target sequence should proceed. In some embodiments, if production is to proceed, information from the in silico analysis, and design analysis (Section II, below) is sent to a production facility. The flow of information from sequence input to production in some embodiments of the present invention is shown FIG. 4.

4. Comprehensive Approach to Whole Genome SNP Analysis and Bioinformatics

As a result of Human Genome Project (HGP), over 35 gigabytes of data is currently available in a large number of public databases, and there is now the potential to quickly and accurately describe the relationship between individual genotype and disease phenotype as never before by analyzing sequence variation. The International SNP Map Working group has constructed a map of 1.4 million candidate SNPs and estimates that two individuals differ at a rate of 1 nucleotide every 1.3 kb (2001). NCBI's dbSNP catalogs over 3 million individual and 1:8 million consensus sequence variations, Japan's SNP db catalogs 117 thousand sequence variations, and HGBASE SNP db catalogs over 65 thousand SNPs. Kruglyak and Nickerson (2001) hypothesized that this collection of sequence variations represents only 11% to 12% of the total human polymorphic nucleotide variation. Therefore, the challenge of discovery is shifting away from discovery to the planning, development, and implementation of clinically relevant assays and studies to provide a synergy between sequence data and large volumes of genotype/phenotype data with effective utilization of a platform of statistical analysis to define disease associations. Additionally, developing and implementing strategies to convert genomic sequence data of varying quality and completeness into biologically meaningful information will be a key to capitalizing on this wealth of information. While the resources available from the HGP make it possible to pursue this strategy of “targeted genomics,” the efficient integration and interpretation of public databases is a major task and becomes one of the critical features of the post-sequencing era. Coupling the computational analysis of publicly available sequence data with clinical studies is crucial.

Through the in silico sequence analysis pipeline of the present invention, it is possible to mine the data generated by the Human Genome Project and to harvest information to annotate the genomic landscape surrounding each SNP (See FIG. 5). The detailed annotation integrates Medline and OMIM data and is used to populate panels of Third Wave Technologies INVADER assays or other detection assays targeted to address specific questions related to disease gene discovery, disease susceptibility, diagnosis and treatment. The panels are designed to map genes, to characterize novel mutations, to create disease-specific gene expression snapshots, to detect clinically relevant mutations, and to facilitate and direct clinical trials of novel treatments for disease. Allele frequency information is generated for each SNP and provides integration between each SNP and the published genetic and physical maps, as well as test algorithms for the prediction of the functional impact of amino acid changes in cSNPs.

Furthermore, the in silico analysis systems and methods described above allow the rapid development of products such as Analyte-Specific Reagents and In-vitro Diagnostics. Since the in silico analysis integrates sequence and expression data with literature and clinical data (e.g. data is fed back into the data management systems of the present invention) the product development funnel (See, section B.IV) if further promoted (See, FIG. 5).

5. RNA Target Sequence Selection in Gene Expression Analysis

Unlike SNP assays wherein there are only two nucleotide locations to design for (sense and antisense strands at the position of the variation), gene expression (GE) assays can be designed to numerous sites (e.g., from about 100 to several 1000 different sites) in a particular mRNA sequence. Further complicating the design process is determining whether there is any homology between the RNA sequence of interest and any others that may be or are likely to be present in the sample. Homologies between target RNA and non-target RNAs occur not only in closely related gene families, but also when RNAs such as mRNAs have several alternative splice configurations. In some embodiments, the assay is intended to detect all or most members of a set of homologous DNAs or RNAs. In other embodiments, an assay is intended to detect a particular nucleic acid and to avoid detecting any similar or related sequences present in a sample. If significant homologies exist, sequence alignments performed before the assay is designed can identify sequences unique to a particular target from sequences that are shared. SNP variations that occur in the mRNA also need to be considered, as their position in the target region can affect assay performance, and location at or near the probe cleavage site may preclude detection of that particular variant. In some embodiments, this is a preferred effect; in some embodiments it is desirable to avoid this effect.

Strategies for designing INVADER assays for detection of RNA include targeting: i) splice sites, ii) accessible sites, and iii) discrimination sites. The type of bioinformatic analysis performed on a given RNA target sequence depends on the type of design strategy being used for developing the assay.

Bioinformatic analysis in mRNA target sequence selection may include mapping of splice sites within the mRNA sequence, identification of any variations in the mRNA sequence (e.g. single-base changes, insertions, deletions), identification and alignment of splice variants, identification and alignment of closely related genes, homology to and alignment of the corresponding gene in other species, and location of accessible sites (unstructured regions of RNA) via in silico analysis. In some embodiments, sequences are obtained from and compared to information from a public database. In other embodiments, sequences are obtained from a private database and compared to information from a private and/or public database In other embodiments, relevant sequences are collected into a local database for rapid retrieval.

In some embodiments, a fully integrated bioinformatic module includes complete analysis of the RNA target sequence prior to assay design, independent of how the assay will be designed. For example, in some embodiments, the user enters a GenBank NM_accession number and the module retrieves the sequence, compares it to an mRNA sequence database (e.g., using BLAST) to retrieve sequences having a percent identity selected by the user (e.g., a minimum identity of 90%), aligns the target sequence with the retrieved sequences, and then uses subroutines to output positions where there is discrimination (e.g., 2 adjacent nucleotides) compared to the collection of retrieved sequences. In some embodiments, additional subroutines comprise locating completely homologous regions of sequence relative to the collection of retrieved sequences for the design of inclusive assays (e.g., assays designed to detect all members of the collection). In other embodiments, subroutines are implemented that retrieve all known alternatively spliced variants, align them, and output splice junctions and included exons for the design of assays that either inclusively or exclusively detect these variants.

In some embodiments, a subroutine performs a BLAST comparison of the mRNA sequence from one species against other databases for other species. In some embodiments, the output of the bioinformatics module comprises identification of splice sites for each RNA.

In some embodiments, homologies are identified and used to design inclusive (e.g., interspecies) assays For example, single assays can detect human and rat CYP1A1, or mouse and rat GAPDH, etc. Interspecies assays have the benefits of making product development more efficient and less expensive, since two or more assays are developed, packaged, and inventoried for the time and price of one. In some embodiments, homologies are identified and used to design exclusive assays (e.g., assays that will not cross-react between species).

In some embodiments, the output of a bioinformatics module is exported to an INVADERCREATOR module. In some embodiments the information is manually entered into the INVADERCREATOR software, while in other embodiments it is read in, e.g., via a batch file. In preferred embodiments, batch files comprise numerical locations for sequences selected as targets for assay design. In other embodiments, other relevant information for assay design such as full gene names, gene name abbreviations, locations of SNP variations are included in the batch files for direct import into INVADERCREATOR software.

In some embodiments, the user selects a design method after reviewing the contents of the bioinformatics output file. In other embodiments, a pre-selected or default design method based on the content of the output file is automatically selected. In some embodiments, e.g., for design of an exclusive assay, the bioinformatics module exports data having particular information regarding homologous sequences found, e.g., a threshold percentage identity value, and this output information directs the INVADERCREATOR module to default to a discrimination sites design method. In some preferred embodiments, information is cross-referenced in the INVADERLOCATOR software.

In some embodiments, output from an INVADERCREATOR analysis is fed back into the bioinformatics module for further analysis. In some embodiments, the bioinformatics module verifies a design feature, e.g., verifies that the final design selection(s) have the intended inclusivity or exclusivity. In other embodiments, a target selected based on one set of criteria (e.g., exclusivity within the RNAs of a single species) is compared to a database using different criteria (e.g., cross-species homologies). In preferred embodiments, the output of the second analysis in the bioinformatics module is returned to the INVADERCREATOR module and the user is offered the option of altering an aspect of the assay design. In other preferred embodiments, alteration or refinement of the assay design is an automated step based on the output from the informatics analysis.

In some embodiments, inventoried assay sequences are reviewed against newly updated databases. In preferred embodiments, users are notified of new information (e.g., via INVADERLOCATOR software) related to previously characterized target sequences, such as newly identified SNPs or splice variants.

II. Detection Assay Design

There are a wide variety of detection technologies available for determining the sequence of a target nucleic acid at one or more locations. For example, there are numerous technologies available for detecting the presence or absence of SNPs. Many of these techniques require the use of an oligonucleotide to hybridize to the target. Depending on the assay used, the oligonucleotide is then cleaved, elongated, ligated, disassociated, or otherwise altered, wherein its behavior in the assay is monitored as a means for characterizing the sequence of the target nucleic acid. A number of these technologies are described in detail, in Section V, below.

The present invention provides systems and methods for the design of oligonucleotides for use in detection assays. In particular, the present invention provides systems and methods for the design of oligonucleotides that successfully hybridize to appropriate regions of target nucleic acids (e.g., regions of target nucleic acids that do not contain secondary structure) under the desired reaction conditions (e.g., temperature, buffer conditions, etc.) for the detection assay. The systems and methods also allow for the design of multiple different oligonucleotides (e.g., oligonucleotides that hybridize to different portions of a target nucleic acid or that hybridize to two or more different target nucleic acids) that all function in the detection assay under the same or substantially the same reaction conditions. These systems and methods may also be used to design control samples that work under the experimental reaction conditions. The present invention also provides methods for designing sequences for amplifying the target sequence to be detected (e.g. designing PCR primers for multiplex PCR).

While the systems and methods of the present invention are not limited to any particular detection assay, the following description illustrates the invention when used in conjunction with the INVADER assay (Third Wave Technologies, Madison Wis.; See e.g. U.S. Pat. Nos. 5,846,717; 6,090,543; 6,001,567; 5,985,557; 5,994,069, 6,214,545, 6,210,880, and 6,194,880; Lyamichev et al., Nat. Biotech., 17:292 (1999), Hall et al., PNAS, USA, 97:8272 (2000), Agarwal et al., Diagn. Mol. Pathol. 9:158 [2000], Cooksey et al., Antimicrob. Agents Chemother. 44:1296 [2000], Griffin and Smith, Trends Biotechnol., 18:77 [2000], Griffin and Smith, Analytical Chemistry 72:3298 [2000], Hessner et al., Clin. Chem. 46:1051 [2000], Ledford et al., J. Molec. Diagnostics 2,:97 [2000], Lyamichev et al., Biochemistry 39:9523 [2000], Mein et al., Genome Res., 10:330 [2000], Neri et al., Advances in Nucleic Acid and Protein Analysis 3826:117 [2000], Fors et al., Pharmacogenomics 1:219 [2000], Griffin et al., Proc. Natl. Acad. Sci. USA 96:6301 [1999], Kwiatkowski et al., Mol. Diagn. 4:353 [1999], and Ryan et al., Mol. Diagn. 4:135 [1999], Ma et al., J. Biol. Chem., 275:24693 [2000], Reynaldo et al., J. Mol. Biol., 297:511 [2000], and Kaiser et al., J. Biol. Chem., 274:21387 [1999]; and PCT publications WO97/27214, WO98/42873, and WO98/50403, each of which is herein incorporated by reference in their entirety for all purposes) to illustrate preferred features of the present invention) to detect a SNP or other sequence of interest. The INVADER assay provides ease-of-use and sensitivity levels that, when used in conjunction with the systems and methods of the present invention, find use in detection panels, ASRs, and clinical diagnostics. One skilled in the art will appreciate that specific and general features of this illustrative example are generally applicable to other detection assays.

A. INVADER Assay

The INVADER assay provides means for forming a nucleic acid cleavage structure that is dependent upon the presence of a target nucleic acid and cleaving the nucleic acid cleavage structure so as to release distinctive cleavage products (See, FIG. 6). 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced.

The INVADER assay provides detections assays in which the target nucleic acid is reused or recycled during multiple rounds of hybridization with oligonucleotide probes and cleavage of the probes without the need to use temperature cycling (i.e., for periodic denaturation of target nucleic acid strands) or nucleic acid synthesis (i.e., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (Reynaldo, et al., J. Mol. Biol. 97: 511-520 [2000]), multiple probes can hybridize to the same target, allowing multiple cleavages, and the generation of multiple cleavage products.

The INVADER assay, as well as other assays, may also employ degenerate oligonucleotides (e.g. degenerate INVADER and probe oligonucleotides). For example, standard INVADER oligonucleotides and probes may be randomly changed at one more positions such that a set of degenerate INVADER and/or probe oligonucleotides are produced. Degenerate sets of INVADER and probe oligonucleotides are particularly useful for use in conjunction with target sequences that tend to be heavily mutated (e.g. HIV-1 pol gene). Using such degenerate sets of INVADER and probe oligonucleotides allows the presence of target sequences at a particular location to be detected even if the surrounding sequence no longer represent the wild type or expected sequence.

The INVADER assay technology may be used to quantitate mRNA (e.g. without target amplification). Low variability (3-10% coefficient of variation) provides accurate quantitation of less than two-fold changes in mRNA levels. A biplex FRET-based detection format enables simultaneous quantitation of expression from two genes within the same sample. One of these genes can be an invariant housekeeping gene that is used as the internal standard. Normalizing the signals from the gene of interest with the internal standard provides accurate results and obviates the need for replicate samples. A simple and rapid cell lysate sample preparation method can be used with the mRNA INVADER Assay. The combined features of biplex detection and easy sample preparation make this assay readily adaptable for use in high-throughput applications.

In certain embodiments, the INVADER assay (and other detection assays such as TAQMAN) employ an E-TAG label from Aclara Corporation (e.g. as part of the INVADER oligonucleotide, probe oligonucleotide, or the FRET oligonucleotide). E-TAG labeling is particularly useful in muliplex analysis. E-TAG labeling does not require surface immobilization of affinity agents. E-TAG type labeling is described in U.S. Pat. Nos. 5,858,188; 5,883,211; 5,935,401; 6,007,690; 6,043,036; 6,054,034; 6,056,860; 6,074,827; 6,093,296; 6,103,199; 6,103,537; 6,176,962; and 6,284,113, all of which are herein incorporated by reference. In particularly preferred embodiments, the detection assays of the present invention employ labels described in U.S. Pat. No. 6,001,567, herein incorporated by reference (e.g. fluorescent molecule and linker at the 5′ end of an oligonucleotide).

B. Oligonucleotide Design for the INVADER Assay

The application of the INVADER assay is not limited to any particular type of nucleic acid or nucleic acid variations. In some embodiments, oligonucleotides for an INVADER assay are designed to detect a particular SNP. In other embodiments, the oligonucleotides for an assay may be designed to determine the presence or absence of a particular nucleic acid in a sample, e.g., a nucleic acid suspected to be present as a consequence of, for example, transfection, transformation or infection of the source of the sample. In yet other embodiments, the oligonucleotides of an INVADER assay may be designed to provide quantitative information about a particular DNA or RNA sequence.

In some embodiments where an oligonucleotide is designed for use in the INVADER assay, the sequence(s) of interest are entered into the INVADERCREATOR program (Third Wave Technologies, Madison, Wis.). One skilled in the art will appreciate that applicability of aspects of this design system for use in other detection assays. As described above, sequences may be input for analysis from any number of sources, either directly into the computer hosting the INVADERCREATOR program, or via a remote computer linked through a communication network (e.g., a LAN, Intranet or Internet network). For detection of double-stranded nucleic acid, e.g., a gene, the program designs probes for both strands, e.g., the sense and antisense strands. Selection of a particular strand for detection is generally based upon factors that include the ease of synthesis, minimization of secondary structure formation, manufacturability and INVADERCREATOR penalty scores, which have been established by studying probe design performance in the INVADER assay. In some embodiments, the user chooses the strand for sequences to be designed for. In other embodiments, the software automatically selects the strand. By incorporating thermodynamic parameters for optimum probe cycling and signal generation (e.g., Allawi and SantaLucia, Biochemistry, 36:10581 [1997] for DNA duplexes, Sugimoto, et al., Biochemistry 34, 11211 [1995] for RNA/DNA hybrids, or Xia, et al., Biochemistry 37:14719 [1998], for RNA duplexes), oligonucleotide probes may be designed to operate at a pre-selected assay temperature (e.g., 63° C.). Based on these criteria, a final probe set (e.g., primary probes for 2 alleles and an INVADER oligonucleotide for a SNP detection assay, or primary probe, a stacker oligonucleotide, an INVADER oligonucleotide and an ARRESTOR oligonucleotide for an RNA detection assay) is selected.

In some embodiments, the INVADERCREATOR system is a web-based program with secure site access that contains a link to BLAST (available at the National Center for Biotechnology Information, National Library of Medicine, National Institutes of Health website) and that can be linked to RNAstructure (Mathews et al., RNA 5:1458 [1999]), a software program that utilizes mfold (Zuker, Science, 244:48 [1989]). RNAstructure can test the proposed oligonucleotide designs generated by INVADERCREATOR for potential uni- and bimolecular complex formation. INVADERCREATOR is open database connectivity (ODBC)-compliant and uses the Oracle database for export/integration. The INVADERCREATOR system is configured with ORACLE to work well with UNIX systems, as most genome centers are UNIX-based.

In some embodiments, the INVADERCREATOR analysis is provided on a separate server (e.g., a Sun server) so it can handle analysis of large batch jobs. For example, a customer can submit up to 2,000 SNP sequences in one email. The server passes the batch of sequences on to the INVADERCREATOR software, and, when initiated, the program designs detection assay oligonucleotide sets. In some embodiments, probe set designs are returned to the user within 24 hours of receipt of the sequences.

Each INVADER reaction includes at least two target sequence-specific, unlabeled oligonucleotides for the primary reaction: an upstream INVADER oligonucleotide and a downstream Probe oligonucleotide. The INVADER oligonucleotide is generally designed to bind stably at the reaction temperature, while the probe is designed to freely associate and disassociate with the target strand, with cleavage occurring only when an uncut probe hybridizes adjacent to an overlapping INVADER oligonucleotide. In some embodiments, the probe includes a 5′ flap or “arm” that is not complementary to the target, and this flap is released from the probe when cleavage occurs. In some embodiments, the released flap participates as an INVADER oligonucleotide in a secondary reaction. In some embodiments, the INVADER reaction may comprise additional oligonucleotides, such as stacker or ARRESTOR oligonucleotides. In some embodiments, the designed oligonucleotides are submitted as a synthesis order, such that manufacture of each oligonucleotide is initiated at order submission, are tracked through the modules of synthesis and the manufactured set of oligonucleotides are collected into a finished assay product or kit. In other embodiments, the oligonucleotide designs are checked against an inventory of existing oligonucleotides to determine if any of the oligonucleotides of the assay have been previously synthesized (“pre-synthesized” oligonucleotides) and stored. In some embodiments, one or more pre-synthesized oligonucleotides are taken from inventory oligonucleotides and included with newly designed and synthesized oligonucleotides in the finished assay or kit. In other embodiments, new assays or kits are assembled entirely from pre-synthesized oligonucleotides taken from an inventory of oligonucleotides.

In some embodiments, of an INVADERCREATOR program, the program is configured to design oligonucleotides for an assay of a single particular type or purpose (e.g., for SNP detection or RNA quantitation). In other embodiments, an INVADERCREATOR program is configured to allow a user to select, e.g., through a button, check box or menu, from a variety of assay types or purposes. The following discussion provides several examples of how a user interface for an INVADERCREATOR program may be configured. Examples of user interfaces are presented in FIGS. 12 through 14. FIG. 12 provides screens images showing one example of using an INVADERCREATOR program to designs an assay for the detection of a SNP (a SNP INVADERCREATOR, or SIC program module). FIG. 13 provides a selection of screen images showing one example of using an INVADERCREATOR program to design an assay for the detection of an RNA target (an RNA INVADERCREATOR, or RIC program module). FIG. 14 provides a selection of screen images showing one example of using an INVADERCREATOR program to design an assay for the detection of a transgene (a Transgene INVADERCREATOR, or TIC program module).

In some embodiments, screens provide optional selection of any number of modifications (e.g., arms, dyes, detectable moieties) for detection or further manipulation. In some embodiments, an INVADERCREATOR module may be customized for a particular assay, or for the needs of a particular user or customer. For example, if a customer has a particular detection platform requiring that the cleavage products comprise moiety X, an INVADERCREATOR module can be configured such that all assays designed by or for customer X are automatically configured to comprise moiety X, in accordance with the customer's requirements. In some embodiments, a pre-designated design feature cannot be altered by an operator creating a new probe design using the customized INVADERCREATOR module. In other embodiments, a pre-designated design feature may be presented to an operator as a default condition of the design that may be overridden during probe design (e.g., by selecting an alternative configuration through one or more data entry screens).

In one embodiment of an INVADERCREATOR program, the user initiates oligonucleotide design by opening a work screen (e.g., FIGS. 12A, 13A or 14A), e.g., by clicking on an icon on a desktop display of a computer (e.g., a Windows desktop). In some embodiments, the user enters information related to the assay, such as project code, company name, assay name, etc. In some embodiments, the used indicates what species the nucleic acid sequence is from. In some embodiments, the user selects the INVADERCREATOR program module to be used (e.g., SIC, RIC, TIC, etc.), e.g., by clicking a button on the screen. The user enters information related to the target sequence for which an assay is to be designed. In some embodiments, the user enters a target sequence (e.g., FIGS. 12B, 13C, or 14B). In other embodiments, the user enters a code or number that causes retrieval of a sequence from a database. In still other embodiments, additional information may be provided, such as the user's name, an identifying number associated with a target sequence, and/or an order number. In preferred embodiments, the user indicates (e.g. via a check box or drop down menu) that the target nucleic acid is DNA or RNA. In other preferred embodiments, the user indicates the species from which the nucleic acid is derived. In particularly preferred embodiments, the user indicates whether the design is for monoplex (i.e., one target sequence or allele per reaction) or multiplex (i.e., multiple target sequences or alleles per reaction) detection. When the requisite choices and entries are complete, the user starts the analysis process. In one embodiment, the user clicks a “Design It” button to continue.

In some embodiments, the software validates the field entries before proceeding. In some embodiments, the software verifies that any required fields are completed with the appropriate type of information. In other embodiments, the software verifies that the input sequence meets selected requirements (e.g., minimum or maximum length, DNA or RNA content). If entries in any field are not found to be valid, an error message or dialog box may appear. In preferred embodiments, the error message indicates which field is incomplete and/or incorrect. Once a sequence entry is verified, the software proceeds with the assay design.

In some embodiments, the information supplied in the order entry fields specifies what type of design will be created. In preferred embodiments, the target sequence and multiplex check box specify which type of design to create. Design options include but are not limited to SNP assay, Multiplexed SNP assay (e.g., wherein probe sets for different alleles are to be combined in a single reaction), Multiple SNP assay (e.g., wherein an input sequence has multiple sites of variation for which probe sets are to be designed), and Multiple Probe Arm assays.

In some embodiments, the INVADERCREATOR software is started via a Web Order Entry (WebOE) process (i.e., through an Intra/Internet browser interface) and these parameters are transferred from the WebOE via applet <param> tags, rather than entered through menus or check boxes.

In the case of Multiple SNP Designs, the user chooses two or more designs to work with. In some embodiments, this selection opens a new screen view (e.g., a Multiple SNP Design Selection view FIG. 8). In some embodiments, the software creates designs for each locus specified in the target sequence, scoring each, and presents them to the user in this screen view. The user can then choose any two designs to work with. In some embodiments, the user chooses a first and second design (e.g., via a menu or buttons) and clicks a “Design It” button to continue.

To select a probe sequence that will perform optimally at a pre-selected reaction temperature, the melting temperature (Tₘ) of the SNP to be detected is calculated using the nearest-neighbor model and published parameters for DNA duplex formation (Allawi and SantaLucia, Biochemistry, 36:10581 [1997], SantaLucia, Proc Natl Acad Sci U S A., 95(4):1460 [1998]). In embodiments wherein the target strand is RNA, parameters appropriate for RNA/DNA heteroduplex formation may be used. Because the assay's salt concentrations are often different than the solution conditions in which the nearest-neighbor parameters were obtained (1M NaCl and no divalent metals), an adjustment should be made to the value provided for the salt concentration within the melting temperature calculations. This adjustment is termed a ‘salt correction’ SantaLucia, Proc Natl Acad Sci U S A., 95(4):1460 [1998]. Similarly, the presence and concentration of the enzyme influence optimal reaction temperature. One way of compensating for these additional factors is to further vary the salt value in the Tm calculations. As used herein, the term “salt correction” refers to a variation made in the value provided for a salt concentration for the purpose of reflecting the effect on a Tₘ calculation for a nucleic acid duplex of a both an alternative salt effect and a non-salt parameter or condition affecting said duplex. Variation of the values provided for the strand concentrations will also affect the outcome of these calculations. By using a value of 0.5 M NaCl (SantaLucia, Proc Natl Acad Sci U S A, 95:1460 [1998]) and strand concentrations of about 1 M of the probe and 1 fM target, the algorithm used for calculating probe-target melting temperature has been adapted for use in predicting optimal INVADER assay reaction temperatures. For one set of 30 probes, the average deviation between optimal assay temperatures calculated by this method and those experimentally determined is about 1.5° C.

The length of the target-complementary region of a probe (e.g., the probe to a given SNP) is defined by the temperature selected for running the reaction (e.g., 63° C.). Starting from the target base that is paired to the probe nucleotide 5′ of the intended cleavage site (e.g., the position of the variant nucleotide on the target DNA)), and adding on the 3′ end, an iterative procedure is used by which the length of the target-binding region of the probe is increased by one base pair at a time until a calculated optimal reaction temperature (Tₘ plus salt correction to compensate for enzyme effect) matching the desired reaction temperature is reached. For INVADER assays detecting DNA targets, the non-complementary arm of the probe is preferably selected to allow the secondary reaction to cycle at the same reaction temperature. The entire probe oligonucleotide is screened using programs such as mfold (Zuker, Science, 244: 48 [1989]) or Oligo 5.0 (Rychlik and Rhoads, Nucleic Acids Res, 17: 8543 [1989]) for the possible formation of dimer complexes or secondary structures that could interfere with the reaction. The same principles are also followed for INVADER oligonucleotide design. Briefly, starting from the position N on the target DNA, additional residues complementary to the target DNA starting from residue N-1 are then added in the 5′ direction until the stability of the INVADER oligonucleotide-target hybrid exceeds that of the probe (and therefore the planned assay reaction temperature), generally by 15-20° C. The 3′ end of the INVADER oligonucleotide is designed to have a nucleotide not complementary to either allele suspected of being contained in the sample to be tested. The mismatch does not adversely affect cleavage (Lyamichev et al., Nature Biotechnology, 17: 292 [1999]), and it can enhance probe cycling, presumably by minimizing coaxial stabilization effects between the two probes.

It is one aspect of the assay design that all of the probe sequences may be selected to allow the primary and secondary reactions to occur at the same optimal temperature, so that the reaction steps can run simultaneously. In an alternative embodiment, the probes may be designed to operate at different optimal temperatures, so that the reaction steps are not simultaneously at their temperature optima.

In some embodiments, the software provides the user an opportunity to change various aspects of the design including but not limited to: probe, target and INVADER oligonucleotide temperature optima and concentrations; blocking groups; probe arms; dyes, capping groups and other adducts; individual bases of the probes and targets (e.g., adding or deleting bases from the end of targets and/or probes, or changing internal bases in the INVADER and/or probe and/or target oligonucleotides). In some embodiments, changes are made by selection from a menu. In other embodiments, changes are entered into text or dialog boxes. In preferred embodiments, this option opens a new screen (e.g., a Designer Worksheet view, FIG. 9).

In some embodiments, the software provides a scoring system to indicate the quality (e.g., the likelihood of performance) of the assay designs. In one embodiment, the scoring system includes a starting score of points (e.g., 100 points) wherein the starting score is indicative of an ideal design, and wherein design features known or suspected to have an adverse affect on assay performance are assigned penalty values. Penalty values may vary depending on assay parameters other than the sequences, including but not limited to the type of assay for which the design is intended (e.g., DNA, RNA, monoplex, multiplex) and the temperature at which the assay reaction will be performed. The following example provides illustrative scoring criteria for use with some embodiments of the INVADER assay based on an intelligence defined by experimentation.

Examples of design features in assays for DNA detection that may incur score penalties (e.g., SIC and TIC module penalties) include but are not limited to the following [penalty values are indicated in brackets; if there are 2 numbers, the first number is for lower temperature assays (e.g., 62-64° C.), second is for higher temperature assays (e.g., 65-66° C.)]:

1. [20] 3′ four bases of the INVADER oligonucleotide resembles the probe arm, for example:

ARM SEQUENCE PENALTY AWARDED IF INVADER ENDS IN:

| | |
|---|---|
| Arm 1: | |
| CGCGCCGAGG | 5′ . . . GAGGX or 5′ . . . GAGGXX |
| Arm 2: | |
| ATGACGTGGCAGAC | 5′ . . . AGACX or 5′ . . . AGACXX |
| Arm 3: | |
| ACGGACGCGGAG | 5′ . . . GGAGX or 5′ . . . GGAGXX |
| Arm 4: | |
| TCCGCGCGTCC | 5′ . . . GTCCX or 5′ . . . GTCCXX |

2. [100] 3′ five bases of the INVADER oligonucleotide resembles the probe arm, for example:

ARM SEQUENCE PENALTY AWARDED IF INVADER ENDS IN:

| | |
|---|---|
| Arm 1: | |
| CGCGCCGAGG | 5′ . . . CGAGGX or 5′ . . . CGAGGXX |
| Arm 2: | |
| ATGACGTGGCAGAC | 5′ . . . CAGACX or 5′ . . . CAGACXX |
| Arm 3: | |
| ACGGACGCGGAG | 5′ . . . CGGAGX or 5′ . . . CGGAGXX |
| Arm 4: | |
| TCCGCGCGTCC | 5′ . . . CGTCCX or 5′ . . . CGTCCXX |

3. [70] probe has a 5-base stretch containing the polymorphism

4. [60] probe has a 5-base stretch adjacent to the polymorphism

5. [15] probe has a 4-base stretch of Gs containing the polymorphism

6. [50] probe has a 5-base stretch of Gs—penalty added anytime it is infringed

7. [40] INVADER oligonucleotide 6-base stretch is of Gs—additional penalty

8. [90] two or three base sequence repeats at least four times starting in the region +1 to +4 of the probe.

9. [100] degenerate base occurs in the probe four bases from either end.

10. [100] probe hybridizing region is short ≦12 bases regardless of assay temperature.

11. [40] probe hybridizing region is long (≧26 bases).

12. [5] hybridizing region length exceeding 26—per base additional penalty

13, [80] insertion/deletion design with poor discrimination in first 3 bases after probe arm

14. [100] calculated INVADER oligonucleotide Tm<7.5C of probe target Tm

15. [100] a probe has a calculated Tm 2C less than its target Tm

Tie Breaker Rules for SIC Module:

1. If calculated probes Tms differ by more than 2.0C, then pick other strand for design.

2. If target of one strand 8 bases longer than that of other strand, then pick shorter strand.

Examples of design features in assays for RNA detection (e.g., RIC module penalties) that may incur score penalties include but are not limited to the following:

1. [50+25 increment/additional G] probe has 4-G stretch in the INVADER oligonucleotide, probe, or stacker. 2. [70] probe has 5-base stretch containing position 1

3. [60] probe has 5-base stretch containing position 2

4. [90] two or three base sequence repeats at least four times starting at position +1 in the probe

5. [100] probe hybridizing region is short (8 bases with a stacker or ≦12 bases without a stacker)

6. [40+5 increment/base] probe hybridizing region is long (≧17 bases with a stacker or ≧20 bases without a stacker)

7. [100] penultimate 3′ base of the INVADER oligonucleotide matches the 3′ base of the probe arm

In some embodiments, penalties are assessed for location of SNP variations at or near the cleavage site. In other embodiments, penalties are assessed based on cleavage site base preferences (e.g., some enzyme may cleave after more efficiently after particular bases, such as Gs, and penalties may be used when a different base is placed in that location). In still other embodiments, penalties are assessed based on ranking of stacking interactions between a probe 3′ base and a stacking oligonucleotide 5′ base (e.g., in some embodiments, AA stacks may perform better than TT stacks.

In particularly preferred embodiments, temperatures for each of the oligonucleotides in the designs are recomputed and scores are recomputed as changes are made. In some embodiments, score descriptions can be seen by clicking a “descriptions” button. In some embodiments, a BLAST search option is provided. In preferred embodiments, a BLAST search is done by clicking a “BLAST Design” button. In some embodiments, this action brings up a dialog box describing the BLAST process. In preferred embodiments, the BLAST search results are displayed as a highlighted design on a Designer Worksheet.

In some embodiments, a user accepts a design by clicking an “Accept” button. In other embodiments, the program approves a design without user intervention. In preferred embodiments, the program sends the approved design to a next process step (e.g., into production; into a file or database). In some embodiments, the program provides a screen view (e.g., an Output Page, FIG. 10 OLD NUMBER), allowing review of the final designs created and allowing notes to be attached to the design. In preferred embodiments, the user can return to the Designer Worksheet (e.g., by clicking a “Go Back” button) or can save the design (e.g., by clicking a “Save It” button) and continue (e.g., to submit the designed oligonucleotides for production).

In some embodiments, the program provides an option to create a screen view of a design optimized for printing (e.g., a text-only view) or other export (e.g., an Output view, FIG. 11). In preferred embodiments, the Output view provides a description of the design particularly suitable for printing, or for exporting into another application (e.g., by copying and pasting into another application). In particularly preferred embodiments, the Output view opens in a separate window.

One embodiments of a design session using the RIC module for RNA assay design is represented in FIG. 13. The RIC module is shown by way of example; similar steps are followed in the SIC and TIC design modules represented in FIGS. 12 and 14, respectively. RNA assay design in this embodiment of the RIC module may comprise the following steps:
entry of assay information into defined fields (e.g., user, assay name, assay abbreviation, etc.) (FIG. 13A).
user selects species via drop down menu (FIG. 13B).
user selects the RNA design module via RIC button (FIG. 13A).
RNA sequences (including FASTA format) is copied and pasted in (FIG. 13C).
cleavage site based design is indicated (e.g., sites indicated are splice junctions, SNPs, or other any other sites selected by user, for example, using the bioinformatics assessment described above; user can enter multiple sites) (FIG. 13C). Multiple probes can be designed per cleavage site (e.g., 257[3] gives three probes for the design for the 257 site).
Stacking oligonucleotide design format can be selected (e.g., “Has Stacker” button, FIG. 13C).
The user can change the non-complementary 5′ arm on the probe via a drop-down menu (FIG. 13D).
Bases can be added to or deleted from the 5′ end of the INVADER oligonucleotide (FIG. 13E), the 3′ end of the probe (automatically adjusts stacking oligonucleotide position and length to satisfy it temperature setting) (FIG. 13F), and the 3′ end of the stacking oligonucleotide.
On the active design page the user can alter the INVADER oligonucleotide, probe, and stacking oligonucleotide temperatures (e.g., FIG. 13G). Exemplary default settings and actual calculated values are shown (e.g., in a separate window).
On the active design page the user can alter the target, INVADER oligonucleotide, probe, and stacking oligonucleotide concentrations e.g., from default settings (FIG. 13H);
user can select enzymes (e.g., alternative CLEAVASE enzymes) via drop-down menu.
All input cleavage site designs can be shown on the same active design page (FIGS. 13D-H);
and the user can select “Cancel” to go back to a previous screen. When finished making any adjustments to the designs, the user can select the “Design Review” button to get to the Design Review step. Design Review shows all entered assay information, the complete mRNA sequence (5′ to 3′), and the designed INVADER oligonucleotide set for each cleavage site aligned to its corresponding mRNA sequence (displayed here 3′ to 5′) (FIG. 13I);
synthetic target sequences are automatically generated including T7 promoter sequence that would enable generation of the mini-in vitro RNA transcript via a transcription kit and a mixture of the two synthetic target sequences.(e.g., FIG. 13I). Arrestor oligonucleotides are automatically designed for each probe and are fully complementary to the target-specific region of the probe and extend 6 nucleotides into the non-complementary 5′ arm. They appear in the INVADERCREATOR output file and are automatically ordered with all 2′-Ome bases (e.g., FIG. 13I);
an “All” button can be selected to automatically order all oligonucleotides for a given design or individual oligonucleotides can be selected or deselected as desired, and a “Notes” field allows the user to type in any comments related to that particular design.
The user selects either the “Job Submit” or “Printable Page/Job Submit” button to move on to the oligo ordering screen (FIG. 13I).
The user gets a listing of all oligonucleotides that were checked for ordering in the Design Review screen and selects each one to call up the oligo order form for that particular oligonucleotide (FIG. 13J).
An Oligo Request form is queued up for each oligo and the user has the ability to select an oligo type via a drop-down menu, the synthesis scale, purification method, various 5′, 3′, or internal modifications, the ability to select “Other” and input unique modifications not listed in the drop-down menus, the ability to highlight a portion of the sequence and designate and alternative nucleotide chemistry (e.g., 2′-Ome's or phosphorothioates) (13 L-0). In some embodiments, the software is set to automatically accept default values and submit all orders directly from the Design Review screen (e.g., via n “order Oligonucleotides Now” button) without user review of an Oligo Request form.
The user selects the “Submit to Synthesis” button when finished modifying a particular Oligo Request form and then queues up the remaining oligonucleotides in the order one by one and does likewise.

In some embodiments, the RIC module also allows the selection of multiple designs for one cleavage site. For example, entering “257, 257, 257, 512” in the sites box (e.g., on FIGS. 13C for 13P) would give the same three designs for 257 and one for 512. As shown in 13P, one could also enter 257 [2] to create 2 designs to the 257 site. In some embodiments, the user has the ability to modify each design individually in the following steps.

One embodiment of a design session using the TIC module for RNA assay design is represented in FIG. 14.
This is the very first screen of automated order entry, and is the same regardless the format (SNP, RNA, Transgene. To go to Transgene InvaderCreator, click on the “TIC button (FIG. 14A).
In this screen the user can paste the Transgene or Internal control sequence. By filling out a number in the “number of loci” field, the user can choose how many designs he or she wants to see. The number of loci are evenly divided over the entered sequence. In addition to these loci, other cleavage sites can be indicated by bracketing a certain base “[C]”. Also, by inserting a number before the base in the bracketed base, multiple probe arm designs can be made (e.g. “[3C]” would design 3 probes for site “C”, each of which can have its own arm (FIG. 14B).
In this screen all the cleavage sites are shown (in sense and antisense orientation). The score is based on penalty scores also used in SNP IC. A perfect design has score 100. When both sense and antisense have a score of 100, a tiebreaker rule gives the winner one extra point. The computer program automatically picks the top two designs based on score, however the user can override those choices. (FIG. 14C).
This is the design page. In principal it is the same as the SNP Invader creator, with the exception that instead of having a sense and antisense design, you have a 1st and 2nd choice design. (FIG. 14D).
Once the designs have been optimized (ie. bases added or deleted) the user can go to the design review page. From here the oligos can be checked for automatic ordering. This is the top half of that page, the bottom half is on the next slide (14E-F).
   C. RNA INVADER Assay Design.

For each design method, typically three different INVADER oligonucleotide sets would be designed and screened and the best performing set would be selected as the product assay. If sufficient detection was not achieved with the initial 3-site screen, a redesign method could include moving the cleavage site/accessible site 1 or more nucleotides in either direction and/or lower scoring designs not ordered in the initial process could be ordered and tested.

Integration of the various design methods could involve querying the user or having the user select one or more design methods based on the following examples:
Does the mRNA sequence have significant homology to other genes or gene family members? If yes, should the target sequence be detected exclusively or inclusively?
Is the mRNA sequence one of 2 or more alternatively spliced variants? If yes, should the target sequence be detected exclusively or inclusively?
If closely related sequences or alternatively spliced variants are not identified in the sequence analysis (e.g., via the bioinformatics module), should the candidate assays be designed via the splice site or accessible site method?

Alternatively, as described above, these types of questions can be encoded in an algorithm that would automatically determine the best design strategy based on the automated sequence analysis in the bioinformatics module.

Splice site design. If assay specificity and/or performance requirements do not dictate otherwise, assays can be designed at or near splice junctions to completely preclude the possibility of detecting genomic DNA in a sample. Splice site design involves determining the splice junctions within the mRNA, usually via pairwise alignment of the mRNA sequence with the genomic DNA sequence for that gene, and then locating INVADER assay cleavage sites at or near the splice site. Typically, the INVADER oligonucleotide is positioned on one side of the splice junction and the probe and stacking oligonucleotide (if used) are positioned on the other side. Thus, if the oligonucleotides were bound to genomic DNA, the probe and INVADER oligonucleotides would be separated by the intervening intronic sequences, which would preclude formation of the required overlap substrate for the CLEAVASE enzyme.

Accessible site design. Again, if assay specificity and/or performance requirements do not dictate otherwise, assays can also be designed to accessible sites within the mRNA. Accessible sites are unstructured regions of the RNA and those determined experimentally, for example, using RT-ROL (Allawi et al. RNA 7:314 [2001]), usually correlate well with enhanced INVADER RNA assay performance. Accessible sites can also be determined via in silico analysis. For example, the RNA sequence could be folded in m-Fold software and then analyzed in Oligowalk to determine accessible sites in the RNA. A program could be written to automatically output the accessible sites (defined as a region with negative Overall G values for an oligonucleotide binding to that region) for the folded RNA. For example, the program could determine when there were 5 or more consecutive nucleotides with Overall G values of −5 or less, then determine the midpoint of this region, and then output those sites into a file. For example, a 10-base negative G region encompassing target sequence nucleotides 200-210 would correspond to an accessible site at 205.

In either case, accessible site design could be encoded into the INVADERCREATOR module by method A or B.

Method A

Assays could be designed in reverse of the cleavage site design process. The user would specify the precise position of the 3′ end of the probe within an accessible site and the probe would be built out toward the 5′ end to satisfy the preset Tm requirement. Stacking oligonucleotide (if designing in a stacker format) contributions to the probe's Tm would be determined as the probe was being built and the Invader oligonucleotide would be designed after the program finished the probe or probe/stacker design.

Method B

Another method for accessible site design, using the same probe-building algorithm that is used for cleavage site design methods, is as follows. The user could enter the accessible site and the INVADERCREATOR module could shift a defined number of bases (a default shift could be determined) downstream. For example, 200 could be entered as an accessible site, and INVADERCREATOR module would build a design using the existing algorithm for cleavage site 210 if the shift value was 10. Next to the check box for “Stacker Design” could be a check box for “Accessible Site Design”. Next to this check box could be a field in which the user would designate the number of bases to shift. The current “Cleavage Sites” field could say “Design Sites” to generically encompass either design mode (cleavage sites or accessible sites). Users could have the capability to check one or both boxes (e.g. stacker design and accessible site design, accessible site design only, etc.).

Splice variant design. Splice variant assays can be designed in a variety of ways. An inclusive detection assay could be designed to detect a region of sequence (e.g. a particular exon) present in all variants. A particular splice variant could be detected by designing the assay to a unique splice site (e.g. if a 5 exon gene yields a splice variant that excludes exon 3, the assay could be designed to detect the exon 2-exon 4 splice junction). Since specificity of the INVADER RNA assay is primarily linked to discrimination at the cleavage site, even very small exonic sequences (e.g. a few nucleotides) could be distinguished. In some cases, it may be useful to detect not any one particular mRNA variant but to individually quantitate exons and/or splice junctions in a pool of mRNA variants. The quantitation pattern from this type of INVADER RNA assay analysis may correlate with particular cellular processes or metabolic states.

Discrimination site design. Closely-related sequences would be aligned to the input target sequence and an automated analysis could be performed to identify all sites that contain, for example, two or more adjacent base differences for any one sequence from all others in the alignment. Another automated analysis algorithm could determine regions of homology of sufficient size to accommodate an INVADER oligonucleotide probe set that would inclusively detect all closely-related mRNAs. An output of the location of such double base discrimination sites or regions of homology could be reviewed by the user before accessing the INVADERCREATOR module or automatically designed via input of a batch file.

The present invention is not limited to the use of the INVADERCREATOR software. Indeed, a variety of software programs are contemplated and are commercially available, including, but not limited to GCG Wisconsin Package (Genetics computer Group, Madison, Wis.) and Vector NTI (Informax, Rockville, Md.).

In some embodiments, the present invention provides design parameters for combining multiple nucleic acid detection technologies. For example, in some embodiments, INVADER assays or other assays are used in conjunction with amplified nucleic acid obtained by using the polymerase chain reaction (PCR). In some preferred embodiments, PCR is run simultaneously with other assays.

D. TAQMAN Probe and Primer Design

A number of different strategies can be used to design TaqMan (5′ Nuclease assay) Probes. The following-are example of considerations that may be used when designing TAQMAN probes. One consideration is to design PCR primers such that the amplicon size is between 50-150 base pairs. Another consideration is to design PCR primers that have a Tm of around 60° C., with less than 2° C. difference in Tm between forward and reverse primers. Preferred primers have GC % around 40-60% and have three or less consecutive runs of any nucleotide. Preferably, the primers have total lengths of between 18-25 nucleotides in length. PCR Primers are designed to have minimal haripin and minimal dimer formation tendencies (See below). Following selection of the PCR primers, the TAQMAN probe is then chosen from within the amplicon region, and has a Tm of about 10° C. higher than the Tm of the PCR primers (typically, 70° C.). TAQMAN probes should have a 5′ FAM and a 3′ TAMRA (or other labels), and not begin with G. TAQMAN probes may be chosen, for example, by using programs such as OligoWalk to scan through the amplicon sequence and a probe chosen based upon predicted most stable thermodynamic parameters. Moreover, candidate TAQMAN probes can be eliminated which forms more than three consecutive basepairs with the PCR primers.

E. Multiplex PCR Primer Design

The INVADER assay can be used for the detection of single nucleotide polymorphisms (SNPs) with as little as 100-10 ng of genomic DNA without the need for target pre-amplification. However, with more than 80,000 INVADER assays developed and the potential for whole genome association studies involving hundreds of thousands of SNPs, the amount of sample DNA becomes a limiting factor for large-scale analysis. Due to the sensitivity of the INVADER assay on human genomic DNA (hgDNA) without target amplification, multiplex PCR coupled with the INVADER assay requires only limited target amplification (10³-10⁴) as compared to typical multiplex PCR reactions that require extensive amplification (10⁹-10¹²) for conventional gel detection methods. The low level of target amplification used for INVADER assay detection provides for more extensive multiplexing by avoiding amplification inhibition commonly resulting from target accumulation.

In some embodiments, it may be desired to detect related loci in a multiplex PCR reaction. In some such embodiments, the similarity between loci may prevent or complicate detection assay analysis of the sequence, as the detection assay technology may not be able to sufficiently discriminate between the closely related sequences. The present invention provides methods to overcome such problems, by generating a unique target sequence using a nucleic acid amplification technique (e.g., PCR), such that the unique target sequence is tested by the detection assay, rather the original sample (e.g., genomic DNA). This method is compatible with multiplexing, where considerations are made to ensure that amplified target sequence meets several criteria: 1) that the target sequence contains the polymorphism to be analyzed; 2) that the target sequence represents a unique target sequence (i.e., it is the only sequence in the reaction mixture that is detected by a detection assay designed to target the target sequence); and 3) that the target sequence does not contain other polymorphisms that are detected by any of the detection assays present in the multiplex reaction. Suitable detection assay components may be selected with methods similar to those described above for the INVADERCREATOR methods. For example, in some embodiments, the software performs a BLAST alignment of the target sequence used for the SNP assay to find similar sequences in the genome that may generate the cross-reactivity signal. The design of PCR primers with software program should prevent amplification of any of the similar loci except the locus containing the SNP. To avoid pre-amplification of sequences other than the specific SNP sequence, the software performs a BLAST alignment of the sequence amplified with a pair of primers against all other detection assay sequences included in the pool. If cross-reactivity or potential cross-reactivity exists, the set of primers is redesigned or the co-amplified sequences are included in different pools.

The same type of design analysis may be used for detection assays directed at the detection of haplotypes. For example, primers are generated to amplify sets of target sequences that each uniquely contain the polymorphisms to be detected.

In some embodiments, multiplex detection assays are provided in a plurality of arrays. For example, in some embodiments, a first array comprises assays configured for detection directly from genomic DNA and a second array comprises assays configured for pre-amplification of target sequences from genomic DNA prior to detection assay analysis of the target sequence.

In some preferred embodiments, only limited pre-amplification of target sequences is carried out prior to detection by the detection assay. For example, in some embodiments, only a 10⁵-10⁶ fold or less increase in target copy number is obtained prior to detection. This is in contrast to typical PCR reactions where 10¹⁰-10¹² or more fold amplification is utilized in detection reactions. In certain embodiments, 100 genotypes from a single PCR amplification are possible with the methods and systems of the present invention using only 10 ng of genomic DNA (e.g. less than 0.1 ng of human genomic DNA per SNP).

In some embodiments, kits are provided for pre-amplification and detection of target sequences. In some embodiments, the kits comprise amplification primers. For multiplex reactions, the amplification primers may be provided in a single container. The amplification primers may also be packaged with detection assay components. In some embodiments, amplification primers and detection assay components (e.g., INVADER assay components) are provided in a single container (e.g., in a single well of a multiwell plate). In some embodiments, the reaction components are provided in dry form in a reaction chamber. In some such embodiments, the kits are configured to allow reactions to occur where the only thing, that is added to the reaction chamber is a solution containing genomic DNA.

The present invention provides methods and selection criteria that allow primer sets for multiplex PCR to be generated (e.g. that can be coupled with a detection assay, such as the INVADER assay). In some embodiments, software applications of the present invention automated multiplex PCR primer selection, thus allowing highly multiplexed PCR with the primers designed thereby. Using the INVADER Medically Associated Panel (MAP) as a corresponding platform for SNP detection, as shown in PCR primer example 2 (below), the methods, software, and selection criteria of the present invention allowed accurate genotyping of 94 of the 101 possible amplicons (˜93%) from a single PCR reaction. The original PCR reaction used only 10 ng of hgDNA as template, corresponding to less than 150 pg hgDNA per INVADER assay.

The multiplex primer design systems may be employed to design PCR primer sets useful with a particular type of assay, such as the INVADER assay. FIG. 15 illustrates creation of one of the primer pairs (both a forward and reverse primer) for a 101 primer set from sequences available for analysis on the INVADER Medically Associated Panel using one embodiment of the software application of the present invention. FIG. 15A shows a sample input file of a single entry (e.g. shows target sequence information for a single target sequence containing a SNP that is processed the method and software of the present invention). The target sequence information in FIG. 15 includes Third Wave Technologies's SNP#, short name identifier, and sequence with the SNP location indicated in brackets. FIG. 15B shows the sample output file of a the same entry (e.g. shows the target sequence after being processed by the systems and methods and software of the present invention. The output information includes the sequence of the footprint region (capital letters flanking SNP site, showing region where INVADER assay probes hybridize to this target sequence in order to detect the SNP in the target sequence), forward and reverse primer sequences (bold), and their corresponding Tm's.

In some embodiments, the selection of primers to make a primer set capable of multiplex PCR is performed in automated fashion (e.g. by a software application). Automated primer selection for multiplex PCR may be accomplished employing a software program designed as shown by the flow chart in FIG. 17.

Multiplex PCR commonly requires extensive optimization to avoid biased amplification of select amplicons and the amplification of spurious products resulting from the formation of primer-dimers. In order to avoid these problems, the present invention provides methods and software application that provide selection criteria to generate a primer set configured for multiplex PCR, and subsequent use in a detection assay (e.g. INVADER detection assays).

In some embodiments, the methods and software applications of the present invention start with user defined sequences and corresponding SNP locations. In certain embodiments, the methods and/or software application determines a footprint region within the target sequence (the minimal amplicon required for INVADER detection) for each sequence (shown in capital letters in FIG. 15B). The footprint region includes the region where assay probes hybridize, as well as any user defined additional bases extending outward therefore (e.g. 5 additional bases included on each side of where the assay probes hybridize). Next, primers are designed outward from the footprint region and evaluated against several criteria, including the potential for primer-dimer formation with previously designed primers in the current multiplexing set (See, primers in bold in FIG. 15A, and selection steps in FIG. 17). This process may be continued, as shown in FIG. 17, through multiple iterations of the same set of sequences until primers against all sequences in the current multiplexing set can be designed.

Once a primer set is designed for multiplex PCR, this set may be employed, in some embodiments, as shown in the basic workflow scheme shown in FIG. 16. Multiplex PCR may be carried out, for example, under standard conditions using only 10 ng of hgDNA as template. After 10 min at 95° C., Taq (2.5 units) may be added to a 50 ul reaction and PCR carried out for 50 cycles. The PCR reaction may be diluted and loaded directly onto an INVADER MAP plate (3 ul/well) (See FIG. 16). An additional 3 ul of 15 mM MgCl₂ may be added to each reaction on the INVADER MAP plate and covered with 6 ul of mineral oil. The entire plate may then be heated to 95° C. for 5 min. and incubated at 63° C. for 40 min. FAM and RED fluorescence may then be measured on a Cytofluor 4000 fluorescent plate reader and “Fold Over Zero” (FOZ) values calculated for each amplicon. Results from each SNP may be color coded in a table as “pass” (green), “mis-call” (pink), or “no-call” (white) (See, PCR Primer Design Example 2 below).

In some embodiments the number of PCR reactions is from about 1 to about 10 reactions. In some embodiments, the number of PCR reactions is from about 10 to about 50 reactions. In further embodiments, the number of PCR reactions is from about 50 to about 100. In additional embodiments, the number of PCR reactions is greater than 100.

The present invention also provides methods to optimize multiplex PCR reactions (e.g. once a primer set is generated, the concentration of each primer or primer pair may be optimized). For example, once a primer set has been generated and used in a multiplex PCR at equal molar concentrations, the primers may be evaluated separately such that the optimum primer concentration is determined such that the multiplex primer set performs better.

Multiplex PCR reactions are being recognized in the scientific, research, clinical and biotechnology industries as potentially time effective and less expensive means of obtaining nucleic acid information compared to standard, monoplex PCR reactions. Instead of performing only a single amplification reaction per reaction vessel (tube or well of a multi-well plate for example), numerous amplification reactions are performed in a single reaction vessel.

The cost per target is theoretically lowered by eliminating technician time in assay set-up and data analysis, and by the substantial reagent savings (especially enzyme cost). Another benefit of the multiplex approach is that far less target sample is required. In whole genome association studies involving hundreds of thousands of single-nucleotide polymorphisms (SNPs), the amount of target or test sample is limiting for large scale analysis, so the concept of performing a single reaction, using one sample aliquot to obtain, for example, 100 results, versus using 100 sample aliquots to obtain the same data set is an attractive option.

To design primers for a successful multiplex PCR reaction, the issue of aberrant interaction among primers should be addressed. The formation of primer dimers, even if only a few bases in length, may inhibit both primers from correctly hybridizing to the target sequence. Further, if the dimers form at or near the 3′ ends of the primers, no amplification or very low levels of amplification will occur, since the 3′ end is required for the priming event. Clearly, the more primers utilized per multiplex reaction, the more aberrant primer interactions are possible. The methods, systems and applications of the present help prevent primer dimers in large sets of primers, making the set suitable for highly multiplexed PCR.

When designing primer pairs for numerous sites (for example 100 sites in a multiplex PCR reaction), the order in which primer pairs are designed can influence the total number of compatible primer pairs for a reaction. For example, if a first set of primers is designed for a first target region that happens to be an A/T rich target region, these primers will be A/T rich. If the second target region chosen also happens to be an A/T rich target region, it is far more likely that the primers designed for these two sets will be incompatible due to aberrant interactions, such as primer dimers. If, however, the second target region chosen is not A/T rich, it is much more likely that a primer set can be designed that will not interact with the first A/T rich set. For any given set of input target sequences, the present invention-randomizes the order in which primer sets are designed (See, FIG. 17). Furthermore, in some embodiments, the present invention re-orders the set of input target sequences in a plurality of different, random orders to maximize the number of compatible primer sets for any given multiplex reaction (See, FIG. 17). In certain embodiments, the primers are designed such that GC-rich and AT-rich regions are avoided.

The present invention provides criteria for primer design that minimizes 3′ interactions (e.g. 3′ complementarity of primers is avoided to reduce probability of primer-dimer formation), while maximizing the number of compatible primer pairs for a given set of reaction targets in a multiplex design. For primers described as 5′-N[x]-N[x−1]- . . . -N[4]-N[3]-N[2]-N[1]-3′, N[1] is an A or C (in alternative embodiments, N[1] is a G or T). N[2]-N[1] of each of the forward and reverse primers designed should not be complementary to N[2]-N[1] of any other oligonucleotide. In certain embodiments, N[3]-N[2]-N[1] should not be complementary to N[3]-N[2]-N[1] of any other oligonucleotide. In preferred embodiments, if these criteria are not met at a given N[1], the next base in the 5′ direction for the forward primer or the next base in the 3′ direction for the reverse primer may be evaluated as an N[1] site. This process is repeated, in conjunction with the target randomization, until all criteria are met for all, or a large majority of, the targets sequences (e.g. 95% of target sequences can have primer pairs made for the primer set that fulfill these criteria).

Another challenge to be overcome in a multiplex primer design is the balance between actual, required nucleotide sequence, sequence length, and the oligonucleotide melting temperature (Tm) constraints. Importantly, since the primers in a multiplex primer set in a reaction should function under the same reaction conditions of buffer, salts and temperature, they need therefore to have substantially similar Tm's, regardless of GC or AT richness of the region of interest. The present invention allows for primer design that meets minimum Tm and maximum Tm requirements and minimum and maximum length requirements. For example, in the formula for each primer 5′-N[x]-N[x−1]- . . . -N[4]-N[3]-N[2]-N[1]-3′, x is selected such the primer has a predetermined melting temperature (e.g. bases are included in the primer until the primer has a calculated melting temperature of about 50 degrees Celsius). In certain embodiments, each of the primers in a set has the same melting temperature.

Often the products of a PCR reaction are used as the target material for another nucleic acid detection means, such as a hybridization-type detection assays, or the INVADER reaction assays for example. Consideration should be given to the location of primer placement to allow for the secondary reaction to successfully occur, and again, aberrant interactions between amplification primers and secondary reaction oligonucleotides should be minimized for accurate results and data. Selection criteria may be employed such that the primers designed for a multiplex primer set do not react (e.g. hybridize with, or trigger reactions) with oligonucleotide components of a detection assay. For example, in order to prevent primers from reacting with the FRET oligonucleotide of a bi-plex INVADER assay, certain homology criteria is employed. In particular, if each of the primers in the set are defined as 5′-N[x]-N[x−1]- . . . -N[4]-N[3]-N[2]-N[1]-3′, then N[4]-N[3]-N[2]-N[1]-3′ is selected such that it is less than 90% homologous with the FRET or INVADER oligonucleotides. In other embodiments, N[4]-N[3]-N[2]-N[1]-3′ is selected for each primer such that it is less than 80% homologous with the FRET or INVADER oligonucleotides. In certain embodiments, N[4]-N[3]-N[2]-N[1]-3′ is selected for each primer such that it is less than 70% homologous with the FRET or INVADER oligonucleotides.

While employing the criteria of the present invention to develop a primer set, some primer pairs may not meet all of the stated criteria (these may be rejected as errors). For example, in a set of 100 targets, 30 are designed and meet all listed criteria, however, set 31 fails. In the method of the present invention, set 31 may be flagged as failing, and the method could continue through the list of 100 targets, again flagging those sets which do not meet the criteria (See FIG. 17). Once all 100 targets have had a chance at primer design, the method would note the number of failed sets, re-order the 100 targets in a new random order and repeat the design process (See, FIG. 17). After a configurable number of runs, the set with the most passed primer pairs (the least number of failed sets) are chosen for the multiplex PCR reaction (See FIG. 17).

FIG. 17 shows a flow chart with the basic flow of certain embodiments of the methods and software application of the present invention. In preferred embodiments, the processes detailed in FIG. 17 are incorporated into a software application for ease of use (although, the methods may also be performed manually using, for example, FIG. 17 as a guide).

Target sequences and/or primer pairs are entered into the system shown in FIG. 17. The first set of boxes show how target sequences are added to the list of sequences that have a footprint determined (See “B” in FIG. 17), while other sequences are passed immediately into the primer set pool (e.g. PDPass, those sequences that have been previously processed and shown to work together without forming Primer dimers or having reactivity to FRET sequences), as well as DimerTest entries (e.g. pair or primers a user wants to use, but that has not been tested yet for primer dimer or fret reactivity). In other words, the initial set of boxes leading up to “end of input” sort the sequences so they can be later processed properly.

Starting at “A” in FIG. 17, the primer pool is basically cleared or “emptied” to start a fresh run. The target sequences are then sent to “B” to be processed, and DimerTest pairs are sent to “C” to be processed. Target sequences are sent to “B”, where a user or software application determines the footprint region for the target sequence (e.g. where the assay probes will hybridize in order to detect the mutation (e.g. SNP) in the target sequence). This region is generally shown in capital letters in figures, such as FIG. 15B. It is important to design this region (which the user may further expand by defining that additional bases past the hybridization region be added) such that the primers that are designed fully encompass this region. In FIG. 17, the software application INVADER CREATOR is used to design the INVADER oligonucleotide and downstream probes that will hybridize with the target region (although any type of program of system could be used to create any type of probes a user was interested in designing probes for, and thus determining the footprint region for on the target sequence). Thus the core footprint region is then defined by the location of these two assay probes on the target.

Next, the system starts from the 5′ edge of the footprint and travels in the 5′ direction until the first base is reached, or until the first A or C (or G or T) is reached. This is set as the initial starting point for defining the sequence of the forward primer (i.e. this serves as the initial N[1] site). From this initial N[1] site, the sequence of the primer for the forward primer is the same as those bases encountered on the target region. For example, if the default size of the primer is set as 12 bases, the system starts with the bases selected as N[1] and then adds the next 11 bases found in the target sequences. This 12-mer primer is then tested for a melting temperature (e.g. using INVADER CREATOR), and additional bases are added from the target sequence until the sequence has a melting temperature that is designated by the user (e.g. about 50 degrees Celsius, and not more than 55 degrees Celsius). For example, the-system employs the formula 5′-N[x]-N[x−1] . . . -N[4]-N[3]-N[2]-N[1]-3′, and x is initially 12. Then the system adjusts x to a higher number (e.g. longer sequences) until the pre-set melting temperature is found.

The next box in FIG. 17, is used to determine if the primer that has been designed so far will cause primer-dimer and/or fret reactivity (e.g. with the other sequences already in the pool). The criteria used for this determination are explained above. If the primer passes this step, the forward primer is added to the primer pool. However, if the forward primer fails this criteria, as shown in FIG. 17, the starting point (N[1] is moved) one nucleotide in the 5′ direction (or to the next A or C, or next G or T). The system first checks to make sure shifting over leaves enough room on the target sequence to successfully make a primer. If yes, the system loops back and check this new primer for melting temperature. However, if no sequence can be designed, then the target sequence is flagged as an error (e.g. indicating that no forward primer can be made for this target).

This same process is then repeated for designing the reverse primer, as shown in FIG. 17. If a reverse primer is successfully made, then the pair or primers is put into the primer pool, and the system goes back to “B” (if there are more target sequences to process), or goes onto “C” to test DimerTest pairs.

Starting a “C” in FIG. 17 shows how primer pairs that are entered as primers (DimerTest) are processed by the system. If there are no DimerTest pairs, as shown in FIG. 17, the system goes on to “D”. However, if there are DimerTest pairs, these are tested for primer-dimer and/or FRET reactivity as described above. If the DimerTest pair fails these criteria they are flagged as errors. If the DimerTest pair passes the criteria, they are added to the primer set pool, and then the system goes back to “C” if there are more DimerTest pairs to be evaluated, or goes on to “D” if there are no more DimerTest pairs to be evaluated.

Starting at “D” in FIG. 17, the pool of primers that has been created is evaluated. The first step in this section is to examine the number of error (failures) generated by this particular randomized run of sequences. If there were no errors, this set is the best set as maybe outputted to a user. If there are more than zero errors, the system compares this run to any other previous runs to see what run resulted in the fewest errors. If the current run has fewer errors, it is designated as the current best set. At this point, the system may go back to “A” to start the run over with another randomized set of the same sequences, or the pre-set maximum number of runs (e.g. 5 runs) may have been reached on this run (e.g. this was the 5th run, and the maximum number of runs was set as 5). If the maximum has been reached, then the best set is outputted as the best set. This best set of primers may then be used to generate as physical set of oligonucleotides such that a multiplex PCR reaction may be carried out.

Another challenge to be overcome with multiplex PCR reactions is the unequal amplicon concentrations that result in a standard multiplex reaction. The different loci targeted for amplification may each behave differently in the amplification reaction, yielding vastly different concentrations of each of the different amplicon products. The present invention provides methods, systems, software applications, computer systems, and a computer data storage lo medium that may be used to adjust primer concentrations relative to a first detection assay read (e.g. INVADER assay read), and then with balanced primer concentrations come close to substantially equal concentrations of different amplicons. A generalized protocol for such multiplex optimization is presented in FIG. 17.

The concentrations for various primer pairs may be determined experimentally. In some embodiments, there is a first run conducted with all of the primers in equimolar concentrations. Time reads are then conducted. Based upon the time reads, the relative amplification factors for each amplicon are determined. Then based upon a unifying correction equation, an estimate of what the primer concentration should be obtained to get the signals closer within the same time point. These detection assays can be on an array of different sizes (384 well plates).

It is appreciated that combining the invention with detection assays and arrays of detection assays provides substantial processing efficiencies. Employing a balanced mix of primers or primer pairs created using the invention, a single point read can be carried out so that an average user can obtain great efficiencies in conducting tests that require high sensitivity and specificity across an array of different targets.

Having optimized primer pair concentrations in a single reaction vessel allows the user to conduct amplification for a plurality or multiplicity of amplification targets in a single reaction vessel and in a single step. The yield of the single step process is then used to successfully obtain test result data for, for example, several hundred assays. For example, each well on a 384 well plate can have a different detection assay thereon. The results of the single step mutliplex PCR reaction has amplified 384 different targets of genomic DNA, and provides you with 384 test results for each plate. Where each well has a plurality of assays even greater efficiencies can be obtained.

Therefore, the present invention provides the use of the concentration of each primer set in highly multiplexed PCR as a parameter to achieve an unbiased amplification of each PCR product. Any PCR includes primer annealing and primer extension steps. Under standard PCR conditions, high concentration of primers in the order of 1 uM ensures fast kinetics of primers annealing while the optimal time of the primer extension step depends on the size of the amplified product and can be much longer than the annealing step. By reducing primer concentration, the primer annealing kinetics can become a rate limiting step and PCR amplification factor should strongly depend on primer concentration, association rate constant of the primers, and the annealing time.

The binding of primer P with target T can be described by the following model:

$$P + T {\to }^{{k}_{a}} PT \qquad ( 1 )$$

where kₐ is the association rate constant of primer annealing. We assume that the annealing occurs at the temperatures below primer melting and the reverse reaction can be ignored.

The solution for this kinetics under the conditions of a primer excess is well known:
[PT]=T₀(1−e^{-k}^{ₐ}^{ct}) (2)
where [PT] is the concentration of target molecules associated with primer, T₀ is initial target concentration, c is the initial primer concentration, and t is primer annealing time. Assuming that each target molecule associated with primer is replicated to produce full size PCR product, the target amplification factor in a single PCR cycle is

$$Z = \frac{{T}_{0} + [ PT ]}{{T}_{0}} = 2 - {ⅇ}^{- {k}_{a} ct} \qquad ( 3 )$$

The total PCR amplification factor after n cycles is given by
F=Zⁿ=(2−e^{-k}^{ₐ}^{ct})ⁿ (4)

As it follows from equation 4, under the conditions where the primer annealing kinetics is the rate limiting step of PCR, the amplification factor should strongly depend on primer concentration. Thus, biased loci amplification, whether it is caused by individual association rate constants, primer extension steps or any other factors, can be corrected by adjusting primer concentration for each primer set in the multiplex PCR. The adjusted primer concentrations can be also used to correct biased performance of INVADER assay used for analysis of PCR pre-amplified loci. Employing this basic principle, the present invention has demonstrated a linear relationship between amplification efficiency and primer concentration and used this equation to balance primer concentrations of different amplicons, resulting in the equal amplification of ten different amplicons in PCR Primer Design Example 1. This technique may be employed on any size set of multiplex primer pairs. In some embodiments, the PCR primers are unoptimized, and the INVADER assay is employed to detect the amplified products (See, Ohnishi et al., J. Hum. Genet. 46:471-7, 2001, herein incorporated by reference.

i. PCR Primer Design Example 1

The following experimental example describes the manual design of amplification primers for a multiplex amplification reaction, and the subsequent detection of the amplicons by the INVADER assay.

Ten target sequences were selected from a set of pre-validated SNP-containing sequences, available in a TWT in-house oligonucleotide order entry database (see FIG. 18). Each target contains a single nucleotide polymorphism (SNP) to which an INVADER assay had been previously designed. The INVADER assay oligonucleotides were designed by the INVADER CREATOR software (Third Wave Technologies, Inc. Madison, Wis.), thus the footprint region in this example is defined as the INVADER “footprint”, or the bases covered by the INVADER and the probe oligonucleotides, optimally positioned for the detection of the base of interest, in this case, a single nucleotide polymorphism (See FIG. 18). About 200 nucleotides of each of the 10 target sequences were analyzed for the amplification primer design analysis, with the SNP base residing about in the center of the sequence. The sequences are shown in FIG. 18.

Criteria of maximum and minimum probe length (defaults of 30 nucleotides and 12 nucleotides, respectively) were defined, as was a range for the probe melting temperature Tm of 50-60° C. In this example, to select a probe sequence that will perform optimally at a pre-selected reaction temperature, the melting temperature (Tₘ) of the oligonucleotide is calculated using the nearest-neighbor model and published parameters for DNA duplex formation (Allawi and SantaLucia, Biochemistry, 36:10581 [1997], herein incorporated by reference). Because the assay's salt concentrations are often different than the solution conditions in which the nearest-neighbor parameters were obtained (1M NaCl and no divalent metals), and because the presence and concentration of the enzyme influence optimal reaction temperature, an adjustment should be made to the calculated Tₘ to determine the optimal temperature at which to perform a reaction. One way of compensating for these factors is to vary the value provided for the salt concentration within the melting temperature calculations. This adjustment is termed a ‘salt correction’. The term “salt correction” refers to a variation made in the value provided for a salt concentration for the purpose of reflecting the effect on a Tₘ calculation for a nucleic acid duplex of a non-salt parameter or condition affecting said duplex. Variation of the values provided for the strand concentrations will also affect the outcome of these calculations. By using a value of 280 nM NaCl (SantaLucia, Proc Natl Acad Sci U S A, 95:1460 [1998], herein incorporated by reference) and strand concentrations of about 10 pM of the probe and 1 fM target, the algorithm for used for calculating probe-target melting temperature has been adapted for use in predicting optimal primer design sequences.

Next, the sequence adjacent to the footprint region, both upstream and downstream were scanned and the first A or C was chosen for design start such that for primers described as 5′-N[x]-N[x−1]- . . . -N[4]-N[3]-N[2]-N[1]-3′, where N[1] should be an A or C. Primer complementarity was avoided by using the rule that: N[2]-N[1] of a given oligonucleotide primer should not be complementary to N[2]-N[1] of any other oligonucleotide, and N[3]-N[2]-N[1] should not be complementary to N[3]-N[2]-N[1] of any other oligonucleotide. If these criteria were not met at a given N[1], the next base in the 5′ direction for the forward primer or the next base in the 3′ direction for the reverse primer will be evaluated as an N[1] site. In the case of manual analysis, A/C rich regions were targeted in order to minimize the complementarity of 3′ ends.

In this example, an INVADER assay was performed following the multiplex amplification reaction. Therefore, a section of the secondary INVADER reaction oligonucleotide (the FRET oligonucleotide sequence) was also incorporated as criteria for primer design; the amplification primer sequence should be less than 80% homologous to the specified region of the FRET oligonucleotide.

The output primers for the 10-plex multiplex design are shown in FIG. 18). All primers were synthesized according to standard oligonucleotide chemistry, desalted (by standard methods) and quantified by absorbance at A260 and diluted to 50 μM concentrated stock. Multiplex PCR was then carried out using 10-plex PCR using equimolar amounts of primer (0.01 uM/primer) under the following conditions; 100 mM KCl, 3 mM MgCl, 10 mM Tris pH8.0, 200 uM dNTPs, 2.5 U taq, and 10 ng of human genomic DNA (hgDNA) template in a 50 ul reaction. The reaction was incubated for (94 C/30sec, 50 C/44 sec.) for 30 cycles. After incubation, the multiplex PCR reaction was diluted 1:10 with water and subjected to INVADER analysis using INVADER Assay FRET Detection Plates, 96 well genomic biplex, 100 ng CLEAVASE VIII, INVADER assays were assembled as 15 ul reactions as follows; 1 ul of the 1:10 dilution of the PCR reaction, 3 ul of PPI mix, 5 ul of 22.5 mM MgCl2, 6 ul of dH20, covered with 15 ul of Chillout. Samples were denatured in the INVADER biplex by incubation at 95 C for 5 min., followed by incubation at 63 C and fluorescence measured on a Cytofluor 4000 at various timepoints.

Using the following criteria to accurately make genotyping calls (FOZ_FAM+FOZ_RED-2>0.6), only 2 of the 10 INVADER assay calls can be made after 10 minutes of incubation at 63 C, and only 5 of the 10 calls could be made following an additional 50 min of incubation at 63 C (60 min.) (See, FIG. 19A). At the 60 min time point, the variation between the detectable FOZ values is over 100 fold between the strongest signal (FIG. 19A, 41646, FAM_FOZ+RED_FOZ−2=54.2, which is also is far outside of the dynamic range of the reader) and the weakest signal (FIG. 19A, 67356, FAM_FOZ+RED_FOZ−2=0.2). Using the same INVADER assays directly against 100 ng of human genomic DNA (where equimolar amounts of each target would be available), all reads could be made with in the dynamic range of the reader and variation in the FOZ values was approximately seven fold between the strongest (FIG. 19, 53530, FAM_FOZ+RED_FOZ−2=3.1) and weakest (FIG. 19, 53530, FAM_FOZ+RED_FOZ−2=0.43) of the assays. This suggests that the dramatic discrepancies in FOZ values seen between different amplicons in the same multiplex PCR reaction is a function of biased amplification, and not variability attributable to INVADER assay. Under these conditions, FOZ values generated by different INVADER assays are directly comparable to one another and can reliably be used as indicators of the efficiency of amplification.

Estimation of amplification factor of a given amplicon using FOZ values. In order to estimate the amplification factor (F) of a given amplicon, the FOZ values of the INVADER assay can be used to estimate amplicon abundance. The FOZ of a given amplicon with unknown concentration at a given time (FOZm) can be directly compared to the FOZ of a known amount of target (e.g. 100 ng of genomic DNA=30,000 copies of a single gene) at a defined point in time (FOZ₂₄₀, 240 min) and used to calculate the number of copies of the unknown amplicon. In equation 1, FOZm represents the sum of RED_FOZ and FAM_FOZ of an unknown concentration of target incubated in an INVADER assay for a given amount of time (m). FOZ₂₄₀ represents an empirically determined value of RED_FOZ (using INVADER assay 41646), using for a known number of copies of target (e.g. 100 ng of hgDNA≅_{—}30,000 copies) at 240 minutes.
F=((FOZₘ−1)*500/(FOZ₂₄₀−1))*(240/m)ˆ2 (equation 1a)

Although equation 1a is used to determine the linear relationship between primer concentration and amplification factor F, equation 1a′ is used in the calculation of the amplification factor F for the 10-plex PCR (both with equimolar amounts of primer and optimized concentrations of primer), with the value of D representing the dilution factor of the PCR reaction. In the case of a 1:3 dilution of the 50 ul multiplex PCR reaction. D=0.3333.
F=((FOZₘ−2)*500/(FOZ₂₄₀−1)*D)*(240/m)ˆ2 (equation 1a′)

Although equations 1a and 1a′ will be used in the description of the 10-plex multiplex PCR, a more correct adaptation of this equation was used in the optimization of primer concentrations in the 107 plex PCR. In this case, FOZ₂₄₀₌the average of FAM_FOZ₂₄₀+RED_FOZ₂₄₀ over the entire INVADER MAP plate using hgDNA as target (FOZ₂₄₀₌3.42) and the dilution factor D is set to 0.125.
F=((FOZₘ−2)*500/(FOZ₂₄₀−2)*D)*(240/m)ˆ2 (equation 1b)

It should be noted that in order for the estimation of amplification factor F to be more accurate, FOZ values should be within the dynamic range of the instrument on which the reading are taken. In the case of the Cytofluor 4000 used in this study, the dynamic range was between about 1.5 and about 12 FOZ.

Section 3. Linear Relationship Between Amplification Factor and Primer Concentration.

In order to determine the relationship between primer concentration and amplification factor (F), four distinct uniplex PCR reactions were run at using primers 1117-70-17 and 1117-70-18 at concentrations of 0.01 uM, 0.012 uM, 0.014 uM, 0.020 uM respectively. The four independent PCR reactions were carried out under the following conditions; 100 mM KCl, 3 mM MgCl, 10 mM Tris pH 8.0, 200 uM dNTPs using 10 ng of hgDNA as template. Incubation was carried out at (94 C/30 sec., 50 C/20 sec.) for 30 cycles. Following PCR, reactions were diluted 1:10 with water and run under standard conditions using INVADER Assay FRET Detection Plates, 96 well genomic biplex, 100 ng CLEAVASE VIII enzyme. Each 15 ul reaction was set up as follows; 1 ul of 1:10 diluted PCR reaction, 3 ul of the PPI mix SNP#47932, 5 ul 22.5 mM MgCl2, 6 ul of water, 15 ul of Chillout. The entire plate was incubated at 95 C for 5 min, and then at 63 C for 60 min at which point a single read was taken on a Cytofluor 4000 fluorescent plate reader. For each of the four different primer concentrations (0.01 uM, 0.012 uM, 0.014 uM, 0.020 uM) the amplification factor F was calculated using equation 1a, with FOZm=the sum of FOZ_FAM and FOZ_RED at 60 minutes, m=60, and FOZ₂₄₀=1.7. In plotting the primer concentration of each reaction against the log of the amplification factor Log(F), a strong linear relationship was noted (FIG. 20). Using the data points in FIG. 20, the formula describing the linear relationship between amplification factor and primer concentration is described in equation 2:
Y=1.684 X+2.6837 (equation 2a)

Using equation 2, the amplification factor of a given amplicon Log(F)=Y could be manipulated in a predictable fashion using a known concentration of primer (X). In a converse manner, amplification bias observed under conditions of equimolar primer concentrations in multiplex PCR, could be measured as the “apparent” primer concentration (X) based on the amplification factor F. In multiplex PCR, values of “apparent” primer concentration among different amplicons can be used to estimate the amount of primer of each amplicon required to equalize amplification of different loci:
X=(Y−2.6837)/1.68 (equation 2b)

Section 4.Calculation of Apparent Primer Concentrations from a Balanced Multiplex Mix.

As described in a previous section, primer concentration can directly influence the amplification factor of given amplicon. Under conditions of equimolar amounts of primers, FOZm readings can be used to calculate the “apparent” primer concentration of each amplicon using equation 2. Replacing Y in equation 2 with log(F) of a given amplification factor and solving for X, gives an “apparent” primer concentration based on the relative abundance of a given amplicon in a multiplex reaction. Using equation 2 to calculate the “apparent” primer concentration of all primers (provided in equimolar concentration) in a multiplex reaction, provides a means of normalizing primer sets against each other. In order to derive the relative amounts of each primer that should be added to an “Optimized” multiplex primer mix R, each of the “apparent” primer concentrations should be divided into the maximum apparent primer concentration (Xₘₐₓ), such that the strongest amplicon is set to a value of 1 and the remaining amplicons to values equal or greater than 1
R[n]=Xmax/X[n] (equation 3)

Using the values of R[n] as an arbitrary value of relative primer concentration, the values of R[n] are multiplied by a constant primer concentration to provide working concentrations for each primer in a given multiplex reaction. In the example shown, the amplicon corresponding to SNP assay 41646 has an R[n] value equal to 1. All of the R[n] values were multiplied by 0.01 uM (the original starting primer concentration in the equimolar multiplex pcr reaction) such that lowest primer concentration is R[n] of 41646 which is set to 1, or 0.01 uM. The remainder of the primer sets were also proportionally increased as shown in FIG. 21. The results of multiplex PCR with the “optimized” primer mix are described below.

Section 5 Using Optimized Primer Concentrations in Multiplex PCR, Variation in FOZ's Among 10 INVADER Assays are Greatly Reduced.

Multiplex PCR was carried out using 10-plex PCR using varying amounts of primer based on the volumes indicated in FIG. 21 (X[max] was SNP41646, setting 1 x=0.01 uM/primer). Multiplex PCR was carried out under conditions identical to those used in with equimolar primer mix; 100 mMKCl, 3 mMMgCl, 10 mM Tris pH8.0, 200 uM dNTPs, 2.5 U taq, and 10 ng of hgDNA template in a 50 ul reaction. The reaction was incubated for (94 C/30 sec, 50 C/44 sec.) for 30 cycles. After incubation, the multiplex PCR reaction was diluted 1:10 with water and subjected to INVADER analysis. Using INVADER Assay FRET Detection Plates, (96 well genomic biplex, 100 ng CLEAVASE VIII enzyme), reactions were assembled as 15 ul reactions as follows; 1 ul of the 1:10 dilution of the PCR reaction, 3 ul of the appropriate PPI mix, 5 ul of 22.5 mM MgCl2, 6 ul of dH20. An additional 15 ul of CHILL OUT was added to each well, followed by incubation at 95 C for 5 min. Plates were incubated at 63 C and fluorescence measured on a Cytofluor 4000 at 10 min.

Using the following criteria to accurately make genotyping calls (FOZ_FAM+FOZ_RED−2>0.6), all 10 of 10 (100%) INVADER calls can be made after 10 minutes of incubation at 63 C. In addition, the values of FAM+RED−2 (an indicator of overall signal generation, directly related to amplification factor (see equation 2)) varied by less than seven fold between the lowest signal (FIG. 22, 67325, FAM+RED−2=0.7) and the highest (FIG. 22, 47892, FAM+RED−2=4.3).

ii. PCR Primer Design Example 2

Using the TWT Oligo Order Entry Database, 144 sequences of less than 200 nucleotides in length were obtained with SNP annotated using brackets to indicate the SNP position for each sequence (e.g. NNNNNNN[N_{(wt)}/N₍ₘₜ₎]NNNNNN). In order to expand sequence data flanking the SNP of interest, sequences were expanded to approximately 1 kB in length (500 nts flanking each side of the SNP) using BLAST analysis. Of the 144 starting sequences, 16 could not expanded by BLAST, resulting in a final set of 128 sequences expanded to approximately 1 kB length (See, FIG. 23). These expanded sequences were provided to the user in Excel format with the following information for each sequence; (1) TWT Number, (2) Short Name Identifier, and (3) sequence (see FIG. 23). The Excel file was converted to a comma delimited format and used as the input file for Primer Designer INVADER CREATOR v1.3.3. software (this version of the program does not screen for FRET reactivity of the primers, nor does it allow the user to specify the maximum length of the primer). INVADER CREATOR Primer Designer v1.3.3., was run using default conditions (e.g. minimum primer size of 12, maximum of 30), with the exception of Tm_{low} which was set to 60 C. The output file (see FIG. 24, bottom of each sheet shows footprint region in upper case letters and SNP in brackets) contained 128 primer sets (256 primers, See FIG. 25), four of which were thrown out due to excessively long primer sequences (SNP#47854, 47889, 54874, 67396), leaving 124 primers sets (248 primers) available for synthesis. The remaining primers were synthesized using standard procedures at the 200 nmol scale and purified by desalting. After synthesis failures, 107 primer sets were available for assembly of an equimolar 107-plex primer mix (214 primers, See FIG. 25). Of the 107 primer sets available for amplification, only 101 were present on the INVADER MAP plate to evaluate amplification factor.

Multiplex PCR was carried out using 101-plex PCR using equimolar amounts of primer (0.025 uM/primer) under the following conditions; 100 mMKCl, 3 mM MgCl, 10 mM Tris pH8.0, 200 uM dNTPs, and 10 ng of human genomic DNA (hgDNA) template in a 50 ul reaction. After denaturation at 95 C for 10 min, 2.5 units of Taq was added and the reaction incubated for (94 C/30 sec, 50 C/44 sec.) for 50 cycles. After incubation, the multiplex PCR reaction was diluted 1:24 with water and subjected to INVADER assay analysis using INVADER MAP detection platform. Each INVADER MAP assay was run as a 6 ul reaction as follows; 3 ul of the 1:24 dilution of the PCR reaction (total dilution 11:8 equaling D=0.125), 3 ul of 15 mM MgCl2 covered with covered with 6 ul of CHILLOUT. Samples were denatured in the INVADER MAP plate by incubation at 95 C for 5 min., followed by incubation at 63 C and fluorescence measured on a Cytofluor 4000 (384 well reader) at various timepoints over 1160 minutes. Analysis of the FOZ values calculated at 10, 20, 40, 80, 160 min. shows that correct calls (compared to genomic calls of the same DNA sample) could be made for 94 of the 101 amplicons detectable by the INVADER MAP platform (FIG. 26 and FIG. 27). This provides proof that the INVADER CREATOR Primer Designer software can create primer sets which function in highly multiplex PCR.

In using the FOZ values obtained throughout the 160 min. time course, amplification factor F and R[n] were calculated for each of the 101 amplicons (FIG. 28). R[nmax] was set at 1.6, which although Low end corrections were made for amplicons which failed to provide sufficient FOZm signal at 160 min., assigning an arbitrary value of 12 for R[n]. High end corrections for amplicons whose FOZm values at the 10 min. read, an R[n] value of 1 was arbitrarily assigned. Optimized primer concentrations of the 101-plex were calculated using the basic principles outlined in the 10-plex example and equation 1b, with an R[n] of 1 corresponding to 0.025 uM primer (see FIG. 15 for various primer concentrations). Multiplex PCR was under the following conditions; 100 mMKCl, 3 mM MgCl, 10 mM Tris pH8.0, 200 uM dNTPs, and 10 ng of human genomic DNA (hgDNA) template in a 50 ul reaction. After denaturation at 95 C for 10 min, 2.5 units of Taq was added and the reaction incubated for (94 C/30 sec, 50 C/44 sec.) for 50 cycles. After incubation, the multiplex PCR reaction was diluted 1:24 with water and subjected to INVADER analysis using INVADER MAP detection platform. Each INVADER MAP assay was run as a 6 ul reaction as follows; 3 ul of the 1:24 dilution of the PCR reaction (total dilution 1:8 equaling D=0.125), 3 ul of 15 mM MgCl2 covered with covered with 6 ul of CHILLOUT. Samples were denatured in the INVADER MAP plate by incubation at 95 C for 5 min., followed by incubation at 63 C and fluorescence measured on a Cytofluor 4000 (384 well reader) at various timepoints over 160 minutes. Analysis of the FOZ values was carried out at 10, 20, and 40 min. and compared to calls made directly against the genomic DNA. Shown in FIG. 26, is a comparison between calls made at 10 min. with a 101-plex PCR with the equimolar primer concentrations versus calls that were made at 10 min. with a 101-plex PCR run under optimized primer concentrations. Additional data for this example is shown in FIGS. 29a, 29b, and 30). Under equimolar primer concentration, multiplex PCR results in only 50 correct calls at the 10 min time point, where under optimized primer concentrations multiplex PCR results in 71 correct calls, resulting in a gain of 21 (42%) new calls. Although all 101 calls could not be made at the 10 min timepoint, 94 calls could be made at the 40 min. timepoint suggesting the amplification efficiency of the majority of amplicons had improved. Unlike the 10-plex optimization that only required a single round of optimization, multiple rounds of optimization may be required for more complex multiplexing reactions to balance the amplification of all loci.

Additional primers for CYP2D6 are shown in FIG. 31. FIG. 32 shows one protocol for multiplex optimization.

F. Sample Preparation Component Design

In some embodiments, genomic DNA that contains a target sequence to be analyzed by the detection assay is used as a starting material for the detection assay. In some such embodiments, it may be desirable to amplify the one or more regions of the genomic DNA (e.g., to generate a plurality of target sequences to be detected). The present invention is not limited by the nature of the amplification technology employed. Amplification techniques include, but are not limited to, PCR and the technologies disclosed in U.S. Pat. Nos. 6,345,514 and 6,221,635, as well as foreign patents and applications, EP1113082, WO200146463, WO200146462, JP2001149097, JP 2001136954, and JP2001008660, herein incorporated by reference in their entireties. In certain embodiments, Rubicon OmniPlex technology is employed for sample preparation. Rubicon OmniPlex technology (See e.g., U.S. Pat. No. 6,197,557, herein incorporated by reference in its entirety) reformats naturally occurring chromosomes into new molecules called Plexisomes. Plexisomes represent the complete genome as amplifiable DNA units of equal length that function as a molecular relational database from which the genetic information can be more quickly and accurately recovered. Use of the technology avoids PCR amplification for sample preparation and for genotyping and haplotyping for gene discovery, pharmacogenomics, and diagnostics by providing highly multiplexing and sample amplification. In preferred embodiments, all the various components for running any of these sample preparation methods are included in a kit (e.g. with at least a portion of a detection assay).

III. Detection Assay Production

The present invention provides a high-throughput detection assay production system, allowing for high-speed, efficient production of thousands of detection assays. The high-throughput production systems and methods allow sufficient production capacity to facilitate full implementation of the funnel process described above-allowing comprehensive of all known (and newly identified) markers. FIG. 98 shows a general overview of the oligonucleotide production and processing systems of the present invention.

In some embodiments of the present invention, oligonucleotides and/or other detection assay components (e.g., those designed by the INVADERCREATOR software and directed to target sequences analyzed by the in silico systems and methods) are synthesized. In preferred embodiments, oligonucleotide synthesis is performed in an automated and coordinated manner. As discussed in more detail below, in some embodiments, produced detection assay are tested against a plurality of samples representing two or more different individuals or alleles (e.g., samples containing sequences from individuals with different ethnic backgrounds, disease states, etc.) to demonstrate the viability of the assay with different individuals. In some embodiments, the systems of the present invention allow at least 300 detection assays to be produced per day. In other embodiments, the systems of the present invention allow at least 1000, or at least 2000 detection assays to be produced per day.

In some embodiments, the present invention provides an automated DNA production process. In some embodiments, the automated DNA production process includes an oligonucleotide synthesizer component and an oligonucleotide processing component. In some embodiments, the oligonucleotide production component includes multiple components, including but not limited to, an oligonucleotide cleavage and deprotection component, an oligonucleotide purification component, an oligonucleotide dry down component; an oligonucleotide de-salting component, an oligonucleotide dilute and fill component, and a quality control component. In some embodiments, the automated DNA production process of the present invention further includes automated design software and supporting computer terminals and connections, a product tracking system (e.g., a bar code system), and a centralized packaging component. In some embodiments, the components are combined in an integrated, centrally controlled, automated production system. The present invention thus provides methods of synthesizing several related oligonucleotides (e.g., components of a kit) in a coordinated manner. The automated production systems of the present invention allow large-scale automated production of detection assays for numerous different target sequences.

In certain embodiments, detection assays are produced in an in-line fashion, such that the synthesized and processed oligonucleotides remain in the same columns and/same holder (e.g. 96 or 384 well plate). In this regard, human and machine interaction with the oligonucleotides being manufactured is minimized.

In certain embodiments, the various production components (e.g. oligonucleotide synthesis component and the various oligonucleotide processing components) are grouped at a single manufacturing location. In different embodiments, the various components are not grouped. For example, the Inventory Control component may be in one location (e.g. closer to a base of customers, or closer to a particular supplier) while the synthesis components are in another location, and many of the processing components are in a third location. This type of remote manufacturing is made possible, for example, by the data management systems of the present invention that allow product orders and inventory for individual assays, and individual components of assays to be tracked. Also, the production and processing facilities may be grouped for ease of use, but there may be multiple locations each producing a different component of an assay. Again, the data management systems of the present invention allow these assay components be separately tracked and assembled in finished assays.

A. Oligonucleotide Synthesis Component

Once a particular oligonucleotide sequence or set of sequences has been chosen, sequences are sent (e.g., electronically) to a high-throughput oligonucleotide synthesizer component. In some preferred embodiments, the high-throughput synthesizer component contains multiple DNA synthesizers.

In some embodiments, the synthesizers are arranged in banks. For example, a given bank of synthesizers may be used to produce one set of oligonucleotides (e.g., for an INVADER or PCR reaction). The, present invention is not limited to any one synthesizer. Indeed, a variety of synthesizers are contemplated, including, but not limited to MOSS EXPEDITE 16-channel DNA synthesizers (PE Biosystems, Foster City, Calif.), OligoPilot (Amersham Pharmacia,), the 3900 and 3948 48-Channel DNA synthesizers (PE Biosystems, Foster City, Calif.), POLYPLEX (Genemachines), 8909 EXPEDITE, Blue Hedgehog (Metabio), MerMade (BioAutomation, Plano, Tex.), Polygen (Distribio, France), PrimerStation 960 (Intelligent Bio-Instruments, Cambridge, Mass.), and the high-throughput synthesizer described in PCT Publication WO 01/41918. In some embodiments, synthesizers are modified or are wholly fabricated to meet physical or performance specifications particularly preferred for use in the synthesis component of the present invention. In some embodiments, two or more different DNA synthesizers are combined in one bank in order to optimize the quantities of different oligonucleotides needed. This allows for the rapid synthesis (e.g., in less than 4 hours) of an entire set of oligonucleotides (all the oligonucleotide components needed for a particular assay, e.g., for detection of one SNP using an INVADER assay). In certain embodiments, the synthesizers are configured for generating oligonucleotides in 96 or 384 well plates.

In some embodiments the DNA synthesizer component includes at least 100 synthesizers. In other embodiments, the DNA synthesizer component includes at least 200 synthesizers. In still other embodiments, the DNA synthesizer component includes at least 250 synthesizers. In some embodiments, the DNA synthesizers are run 24 hours a day.

1. Synthesizers

A. Exemplary Synthesizers

The present invention provides nucleic acid synthesizers and methods of using and modifying nucleic acid synthesizers. For example, the present invention provides highly efficient, reliable, and safe synthesizers that find use, for example, in high throughput and automated nucleic acid synthesis (e.g. arrays of synthesizers), as well as methods of modifying pre-existing synthesizers to improve efficiency, reliability, and safety.

A problem with currently available synthesizers is the emission of undesirable gaseous or liquid materials that pose health, environmental, and explosive hazards. Such emissions result from both the normal operation of the instrument and from instrument failures. Emissions that result from instrument failures cause a reduction or loss of synthesis efficiency and can provoke further failures and/or complete synthesizer failure. Correction of failures may require taking the synthesizer off-line for cleaning and repair. The present invention provides nucleic acid synthesizers with components that reduce or eliminate unwanted emissions and that compensate for and facilitate the removal of unwanted emissions, to the extent that they occur at all. The present invention also provides waste handling systems to eliminate or reduce exposure of emissions to the users or the environment. Such systems find use with individual synthesizers, as well as in large-scale synthesis facilities comprising many synthesizers (e.g. arrays of synthesizers).

In some particularly preferred embodiments, the present invention provides efficient and safe “open system synthesizers.” Open system synthesizers are contrasted to “closed system synthesizers” in that the reagent delivery, synthesis compartments, and waste extraction for each synthesis column are riot contained in a system that remains physically closed (i.e., closed from both the ambient environment and from the other synthesis columns in the same instrument) for the duration of the synthesis run. For example, in a closed system, tubing (or other means) provided for the addition and removal of reagent to each reaction compartment or synthesis column is generally fixed to the column with a coupling that is sealed to isolate the contents of that system from its surroundings. In contrast, in an open system, the dispensing and/or removal of reagent may be through means that are not physically coupled to the reaction compartment.

Further, a common dispensing or waste removal means may be shared by multiple reaction compartments, such that each compartment sharing the means is serviced in turn. An example of an “open system synthesizer” is described in PCT Publication WO 99/65602, herein incorporated by reference in its entirety. This publication describes a rotary synthesizer for parallel synthesis of multiple oligonucleotides. The tubing that supplies the synthesis reagents to the synthesis column does not form a continuous closed seal to the synthesis columns. Instead, the rotor turns, exposing the synthesis columns, in series, to the dispense lines, which inject synthesis reagents into the synthesis column. Open synthesizers offer advantages over closed synthesizers for the simultaneous production of multiple oligonucleotides. For example, a large number of independent synthesis columns, each intended to produce a distinct oligonucleotide, are exposed to a smaller number of dedicated reagent dispensers (e.g., four dedicated dispensers for each of the nucleotides). Open systems also provide easy access to synthesis columns, which can be added or removed without detaching any otherwise fixed connections to reagent dispensing tubing.

While open synthesizers have advantages for the production of oligonucleotides, they suffer from increased problems of emissions and failures. The direct exposure of the columns to their surroundings and the non-continuous path of reagents increases the number of points at which gaseous and liquid emissions occur, thereby increasing the release of unwanted emissions to the atmosphere and leakage within the synthesizer. Many synthesizers carry out reagent delivery, nucleic acid synthesis, and waste disposal under pressurized conditions. Open systems have frequent problems with loss of pressure, resulting in instrument failures and/or loss of synthesis efficiency. The open system synthesizers of the present invention dramatically reduce instrument failures and the corresponding emissions.

Whether a system used is open or closed, oligonucleotide synthesis involves the use of an array of hazardous materials, including but not limited to methylene chloride, pyridine, acetic anhydride, 2,6-lutidine, acetonitrile, tetrahydrofurane, and toluene. These reagents can have a variety of harmful effects on those who may be exposed to them. They can be mildly or extremely irritating or toxic upon short-term exposure; several are more severely toxic and/or carcinogenic with long-term exposure. Many can create a fire or explosion hazard if not properly contained. In addition, many of these chemicals must be assessed for emissions from normal operations, e.g for determining compliance with OSHA or environmental agency standards. Malfunction of a system, e.g., as recited above, increases such emissions, thereby increasing the risk of operator exposure, and increasing the risk that an instrument may need to be shut down until risk to an operator is reduced and until any regulatory requirements for operation are met.

Emission or leakage of reagents during operation can have consequences beyond risks to personnel and to the environment. As noted above, instruments may need to be removed from operation for cleaning, leading to a temporary decrease in production capacity of a synthesis facility. Further, any emission or leakage may cause damage to parts of the instrument or to other instruments or aspects of the facility, necessitating repair or replacement of any such parts or aspects, increasing the time and cost of bringing an instrument back into operation. Failure to address emissions or leakage concerns may lead to additional expenses for operation of a facility, e.g., costs for increased or improved fire or explosion containment measures, and addition of costs associated with the elimination of any instrument systems or wiring that have not been determined to be safe for use in such hazardous locations (e.g., by reference to controlling codes, such as electrical codes, or codes covering operations in the presence of flammable and combustible liquids).

The synthesizers of the present invention provide a number of novel features that dramatically improve synthesizer performance and safety compared to available synthesizers. These novel features work both independently and in conjunction to provide enhanced performance. For example, in some embodiments, the synthesizers of the present invention prevent loss of pressure during synthesis and waste disposal. By preventing loss of pressure, synthesis columns are purged properly and do not overflow during subsequent synthesis steps. Thus, prevention of pressure loss further prevents liquid overflow and instrument contamination. Additionally, in some embodiments, sufficient pressure differentials are maintained across all columns to allow efficient synthesis and purging without instrument failure. For example, regardless of whether synthesis columns are actively involved in a particular round of synthesis (e.g., short oligonucleotides will be completed prior to the completion of longer oligonucleotides and will not be actively synthesized during the later round of synthesis), sufficient pressure differentials are maintained to allow reagent delivery and purging from the active columns. A number of additional features of the synthesizers of the present invention are described in detail below.

In addition to providing efficient synthesizers, the present invention provides methods for modifying existing synthesizers to improve their efficiency. For example, one or more of the novel components of the present invention may be added into or substituted into existing synthesizers to improve efficiency and performance.

The present invention further provides means of reducing exposure of operators and the environment to synthesis reagents and waste. In one embodiment, the present invention reduces exposure by improving collection and disposal of emissions that occur during the normal operation of various synthesis instruments. In another embodiment, the present invention reduces exposure by improving aspects of the instrument to reduce risk of malfunctions leading to reagent escape from the system, e.g., through leakage, overflow or other spillage.

While the present invention will be described with reference to several specific embodiments, the description is illustrative of the present invention and is not to be construed as limiting the invention. Various modifications to the present invention can be made without departing from the scope and spirit of the present invention. For example, much of the following description is provided in the context of an open system synthesizer (see, e.g., WO99/65602). However, the invention is not limited to open system synthesizers.

In preferred embodiments, the present invention provides open-system solid phase synthesizers that are suitable for use in large-scale polymer production facilities. Each synthesizer is itself capable of producing large volumes of polymers. However, the present invention provides systems for integrating multiple synthesizers into a production facility, to further increase production capabilities.

FIG. 33 illustrates a synthesizer 1. The synthesizer 1 is designed for building a polymer chain by sequentially adding polymer units to a solid support in a liquid reagent. The liquid reagents used for synthesizing oligonucleotides may vary, as the successful operation of the present invention is not limited to any particular coupling chemistry. Examples of suitable liquid reagents include, but are not limited to: Acetonitrile (wash); 2.5% dichloroacetic acid in methylene chloride (deblock); 3% tetrazole in acetonitrile (activator); 2.5% cyanoethyl phosphoramidite in acetonitrile (A, C, G, T); 2.5% iodine in 9% water, 0.5% pyridine, 90.5% THF (oxidizer); 10% acetic anhydride in tetrahydrofuran (CAP A); and 10% 1-methylimidazole, 10% pyridine, 80% THF. Various useful reagents and coupling chemistries are described in U.S. Pat. No. 5,472,672 to Bennan, and U.S. Pat. No. 5,368,823 to McGraw et al. (both of which are herein incorporated by reference in their entireties).

The solid support generally resides within a synthesis column and various liquid reagents are sequentially added to the synthesis column. Before an additional liquid reagent is added to a synthesis column, the previous liquid reagent is preferably purged from the synthesis column. Although the synthesizer 1 is particularly suited for building nucleic acid sequences, the synthesizer 1 is also configured to build any other desired polymer chain or organic compound (e.g. peptide sequences).

The synthesizer 1 preferably comprises at least one bank of valves and at least one bank of synthesis columns. Within each bank of synthesis columns, there is at least one synthesis column for holding the solid support and for containing a liquid reagent such that a polymer chain can be synthesized. Within the bank of valves, there are preferably a plurality of valves configured for selectively dispensing a liquid reagent into one of the synthesis columns. The synthesizer 1 is preferably configured to allow each bank of synthesis columns to be selectively purged of the presently held liquid reagent. In particularly preferred embodiments, the synthesizer of the present invention is configured to allow synthesis columns within a bank to be purged even when not all of the synthesis columns contain liquid reagents (e.g. only a portion of the synthesis columns in a bank received a liquid reagent (i.e. “active”), while the remaining synthesis columns are no longer receiving liquid reagent (i.e. “idle”). For example, in some preferred embodiments of the present invention, the design of the material in the synthesis columns allows idle columns to resist the downward pressure of gas, thus making this pressure available to purge the synthesis columns that contain liquid reagent. Additional banks of valves provide the synthesizer 1 with greater flexibility. For example, each bank of valves can be configured to distribute liquid reagents to a particular bank of synthesis columns in a parallel fashion to minimize the processing time.

Multiple banks of valves can also be configured to distribute liquid reagents to a particular bank of synthesis columns in series. This allows the synthesizer 1 to hold a larger number of different reagents, thus being able to create varied nucleic acid sequences (e.g. 48 oligonucleotides, each with a unique sequence).

FIG. 33 illustrates a top view of a rotary synthesizer 1. As illustrated in FIG. 33, the synthesizer 1 includes a base 2, a cartridge 3, a first bank of synthesis columns 4, a second bank of synthesis columns 5, a plurality of dispense lines 6, a plurality of fittings 7 (a first bank of fittings 13, and a second bank of fittings 14), a first bank of valves 8 and a second bank of valves 9. Within each of the banks of valves 8 and 9, there is preferably at least one valve. Within each of the banks of synthesis columns 4 and 5, there is preferably at least one synthesis column. Each of the valves is capable of selectively dispensing a liquid reagent into one of the synthesis columns. Each of the synthesis columns is preferably configured for retaining a solid support such as polystyrene or CPG and holding a liquid reagent. Further, as each liquid reagent is sequentially deposited within the synthesis column and sequentially purged therefrom, a polymer chain is generated (e.g. nucleic acid sequence).

Preferably, there is a plurality of reservoirs, each containing a specific liquid reagent to be dispensed to one of the plurality of valves 8 or 9. Each of the valves within the first bank and second bank of valves 8 and 9, is coupled to a corresponding reservoir. Each of the plurality of reservoirs is pressurized (e.g. by argon gas). As a result, as each valve is opened, a particular liquid reagent from the corresponding reservoir is dispensed to a corresponding synthesis column. Each of the plurality of dispense lines 6 is coupled to a corresponding one of the valves within the first and second banks of valves 8 and 9. Each of the plurality of dispense lines 6 provides a conduit for transferring a liquid reagent from the valve to a corresponding synthesis column. Each one of the plurality of dispense lines 6 is preferably configured to be flexible and semi-resilient in nature. In preferred embodiments, the dispense lines of the present invention have a large bore size to prevent clogging. In preferred embodiments, the internal diameter of the dispense tube is at least 0.25 mm. In other embodiments, the internal diameter of the tube is at least 0.50 mm or at least 0.75 mm. In some embodiments, the internal diameter of the tube is greater than or equal to 1.0 mm (e.g. 1.0 mm, or 1.2 mm, or 1.4 mm). Preferably, the plurality of dispense lines 6 are each made of a material such as PEEK, glass, or coated with TEFLON or Parlene, or coated/uncoated stainless steel or other metallic material. Of course other materials may also be used. For example, useful characteristics of the material used for the dispense lines would be resistance to degradation by the liquid reagents, minimal “wetting” by the liquid reagents, ease of fabrication, relative rigidity, and ability to be produced with a smooth surface finish. Metallic tubing (e.g. stainless steel), benefit from electropolishing to improve the surface finish (e.g. in coated or uncoated application). Another important characteristic of useful dispense lines in the ability to provide a seal between the plurality of valves 10 and the plurality of fittings 7.

Each of the plurality of fittings 7 is preferably coupled to one of the plurality of dispense lines 6. The plurality of fittings 7 are preferably configured to prevent the reagent from splashing outside the synthesis column as the reagent is dispensed from the fitting to a particular synthesis column positioned below the fitting. In preferred embodiments, the fitting includes a nozzle that prevents reagents from drying at the point fluid exits the nozzle (e.g. prevents dried reagents from causing the reagents stream to dispense at angles away from the intended synthesis column). Construction techniques to achieve consistent flow at the discharge point of the liquid reagents is achieved by the use of high quality parts and construction. For example, clean square cuts (without burrs or shavings), or the use of a “drawn tip” (i.e., a tip of reduced diameter at the discharge point). The use of a drawn tip, for example, reduces the wall thickness at the point of discharge, thus reducing the area of the tube wall cross section, providing a smooth transition from the larger portion of the tube (reducing flow resistance) and increases the likelihood of a clean separation of the discharged liquid reagent from the tip of the tube. This clean “snap” of the liquid reagent minimizes the retention of the discharged fluid at the tip, and thus minimizes subsequent build up of any solids (e.g. dried reagent). Additionally, if a sharp cut off of the fluid flow is obtained, the fluid front will actually reside within the confines of the tube after discharge of the desired volume. This minimizes surface evaporation and helps to maintain a clean orifice (e.g. prevent reagent from drying at the tip). Another example of a useful technique to prevent liquid reagent from drying at the discharge point is providing a sleeve or sheath over the dispense line to a point near the tip (dispense point). This sleeve or sheath is particularly useful when employed in conjunction with a relatively flexible dispense line.

As shown in FIG. 33, the first and second banks of valves 8 and 9 each have thirteen valves. In FIG. 33, the number of valves in each bank is merely for exemplary purposes (e.g. other numbers of valves may be employed, like 14, 15, 16, 17, etc.).

Each of the synthesis columns within the first bank of synthesis columns 4 and the second bank of synthesis columns 5 is presently shown resting in one of a plurality of receiving holes 11 within the cartridge 3. Preferably, each of the synthesis columns within the corresponding plurality of receiving holes 11 is positioned in a substantially vertical orientation. Each of the synthesis columns is configured to retain a solid support such as polystyrene or CPG and hold liquid reagent(s). In preferred embodiments, polystyrene is employed as the solid support. Alternatively, any other appropriate solid support can be used to support the polymer chain being synthesized.

During synthesizer operation, each of the valves selectively dispenses a liquid reagent through one of the plurality of dispense lines 6 and fittings 7. The first and second banks of valves 8 and 9 are preferably coupled to the base 2 of the synthesizer 1. The cartridge 3 which contains the plurality of synthesis columns 12 rotates relative to the synthesizer 1 and relative to the first and second banks of valves 8 and 9. By rotating the cartridge 3, a particular synthesis column 12 is positioned under a specific valve such that the corresponding reagent from this specific valve is dispensed into this synthesis column. In preferred embodiments, the cartridge 3 has a home position that allows the synthesizer to be properly aligned before operation (such that the liquid reagent is properly dispensed into the synthesis columns). Further, the first and second banks of valves 8 and 9 are capable of simultaneously and independently dispensing liquid reagents into corresponding synthesis columns.

A cross sectional view of synthesizer 1 is depicted in FIG. 34. As depicted in FIG. 34, the synthesizer 1 includes the base 2, a set of valves 15, a motor 16, a gearbox 17, a chamber bowl 18, a drain plate 19, a drain 20, a cartridge 3, a bottom chamber seal 21, a motor connector 22, a waste tube system 23, a controller 24, and a clear window 25. The valves 15 are coupled to base 2 of the synthesizer 1 and are preferably positioned above the cartridge 3 around the outside edge of the base 2. This set of valves 15 preferably contains fifteen individual valves which each deliver a corresponding liquid reagent in a specified quantity to a synthesis column held in the cartridge 3 positioned below the valves. Each of the valves may dispense the same or different liquid reagents depending on the user-selected configuration. When more than one valve dispenses the same reagent, the set of valves 15 is capable of simultaneously dispensing a reagent to multiple synthesis columns within the cartridge 3. When the valves 15 each contain different reagents, each one of the valves 15 is capable of dispensing a corresponding liquid reagents to any one of the synthesis columns within the cartridge 3.

The synthesizer 1 may have multiple sets of valves. The plurality of valves within the multiple sets of valves may be configured in a variety of ways to dispense the liquid reagents to a select one or more of the synthesis columns. For example, in one configuration, where each set of valves is identically configured, the synthesizer 1 is capable of simultaneously dispensing the same reagent in parallel from multiple sets of valves to corresponding banks of synthesis columns. In this configuration, the multiple banks of synthesis columns may be processed in parallel. In the alternative, each individual valve within multiple sets of valves may contain entirely different liquid reagents such that there is no duplication of reagents among any individual valves in the multiple sets of valves. This configuration allows the synthesizer 1 to build polymer chains requiring a large variety of reagents without changing the reagents associated with each valve.

The motor 16 is preferably mounted to the base 2 through the gear box 17 and the motor connector 22. The chamber bowl 18 preferably surrounds the motor connector 22 and remains stationary relative to the base 2.

The chamber bowl 18 is designed to hold any reagent spilled from the plurality of synthesis columns 12 during the purging process (or the dispensing process). Further, the chamber bowl 18 is configured with a tall shoulder to insure that spills are contained within the bowl 18. The bottom chamber seal 21 preferably provides a seal around the motor connector 22 in order to prevent the contents of the chamber bowl 18 from flowing into the gear box 17 (see FIG. 34). The bottom chamber seal 21 is preferably composed of a flexible and resilient material such as TEFLON (or elastomer which conforms to any irregularities of the motor connector 22). Alternatively, the bottom chamber seal can be composed of any other appropriate material. In particularly preferred embodiments, the bottom chamber seal is composed of material that resists constant contact with liquid reagents (e.g., TEFLON or Parlene). Additionally, the bottom chamber seal 21 may have frictionless properties that allow the motor connector 22 to rotate freely within the seal. For example, coating this flexible material with TEFLON helps to achieve a low coefficient of friction.

The clear window 25 is attached to (formed in) a top cover 30 of the synthesizer 1 and covers the area above the cartridge 3. The top cover 30 of synthesizer 1 seals the top part of the chamber (when in place), and opens up allowing an operator or maintenance person access to the interior of the synthesizer 1. The clear window 25 in top cover 30 allows the operator to observe the synthesizer 1 in operation while providing a pressure sealed environment within the interior of the synthesizer 1. As shown in FIG. 34, there are a plurality of through holes 26 in the clear window 25 to allow the plurality of dispense lines 6 to extend through the clear plate 25 to dispense material into the synthesis columns located in cartridge 3.

The clear window 25 also includes a gas fitting 27 attached therethrough. The gas fitting 27 is coupled to a gas line 28. The gas line 28 preferably continuously emits a stream of inert gas (e.g. Argon) which flows into the synthesizer 1 through the gas fitting 27 and flushes out traces of air and water from the plurality of synthesis columns 12 within the synthesizer 1. Providing the inert gas flow through the gas fitting 27 into the synthesizer 1 prevents the polymer chains being formed within the synthesis columns from being contaminated without requiring the plurality of synthesis columns 12 to be hermetically sealed and isolated from the outside environment.

FIG. 35 shows the cartridge 3 in chamber bowl 18, with the top plate 30 removed, thus revealing the top chamber seal 31. Top chamber seal 31 is designed to provide a tight seal between top plate 30 and chamber bowl 18, such that inert gas applied through clear window 25 does not leak. If the top chamber seal 31 does not function properly, the inert gas leaks out (lowering the pressure in the chamber), thus causing the purge operation (that relies on the pressure on the inert gas) to fail. When the purge operation fails, un-purged columns quickly fill up and overflow. In some embodiments, a V-seal type top chamber seal is employed to prevent leakage of gas. In some embodiments, the hinges and latches on top plate 30 (not shown) are precisely machined to provide balanced forces on the top plate 30, such that the top plate 30 fits tightly over the chamber bowl.

FIG. 36 illustrates a detailed view of a cartridge 3 for synthesizer 1. Preferably, the cartridge 3 is circular in shape such that it is capable of rotating in a circular path relative to the base 2 and the first and second banks of valves 8 and 9. The cartridge 3 has a plurality of receiving holes 11 on its upper surface around the peripheral edge of the cartridge 3. Each of the plurality of receiving holes 11 is configured to hold one of the synthesis columns 12. The plurality of receiving holes 11, as shown on the cartridge 3, is divided up among four banks. A bank 32 illustrates one of the four banks on the cartridge 3 and contains twelve receiving holes, wherein each receiving hole is configured to hold a synthesis column. An exemplary synthesis column 12 is shown being inserted into one of the plurality of receiving holes 11. The total number of receiving holes shown on the cartridge 3 includes forty-eight (48) receiving holes, divided into four banks of twelve receiving holes each. The number of receiving holes and the configuration of the banks of receiving holes is shown on the cartridge 3 for exemplary purposes only. Any appropriate number of receiving holes and banks of receiving holes can be included in the cartridge 3. Preferably, the receiving holes 11 within the cartridge each have a precise diameter for accepting the synthesis columns 12, which also each have a corresponding precise exterior surface 61 (see FIG. 44) to provide a pressure-tight seal when the synthesis columns 12 are inserted into the receiving holes 11. In preferred embodiments, the synthesis column includes a column seal 65 (see FIG. 44), such as a ring seal or a ball seal (e.g., a flexible TEFLON ring that flexes on engagement of the synthesis column in the receiving hole 11). In other preferred embodiments, a seal, such as a ring seal, is provided above or in the receiving holes 11 (see, e.g., FIG. 44).

FIG. 37 depicts an exemplary drain plate 19 of the synthesizer 1. The drain plate 19 is coupled to the motor connector 22 (not shown) through securing holes 33. More specifically, the drain plate 19 is attached to the motor connector 22, which rotates the drain plate 19 while the motor 16 is operating and the gear box 17 is turning. The cartridge 3 and the drain plate 19 are preferably configured to rotate as a single unit. The drain plate 19 is configured to catch and direct the liquid reagents as the liquid reagents are expelled from the plurality of synthesis columns (during the purging process). During operation, the motor 16 is configured to rotate both the cartridge 3 and the drain plate 19 through the gear box 17 and the motor connector 22. The bottom chamber seal 21 allows the motor connector 22 to rotate the cartridge 3 and the drain plate 19 through a portion of the chamber bowl 18 while still containing spilled reagents in the chamber bowl 18. The controller 24 is coupled to the motor 16 to activate and deactivate the motor 16 in order to rotate the cartridge 3 and the drain plate 19. The controller 24 (see FIG. 34) provides embedded control to the synthesizer and controls not only the operation of the motor 16, but also the operation of the valves 15 and the waste tube system 23.

The drain plate 19 has a plurality of securing holes 33 for attaching to the motor connector 22. The drain plate 19 also has a top surface 34 which may, in some embodiments, attach to the underside of the cartridge 3. In other embodiments, a drain plate gasket is provided between the drain plate 19 and cartridge 3 (see below). As stated previously, the cartridge 3 holds the plurality of synthesis columns grouped into a plurality of banks. The drain plate preferably has a collection area corresponding to each of the banks of synthesis columns (e.g. four in FIG. 37 to correspond to the four banks of synthesis columns in cartridge 3). Each of these four collection areas 35, 36, 37 and 38 in FIG. 37, forms a recessed area below the top surface 34 and is designed to contain and direct material flushed from the synthesis columns within the bank above the collection area.

Each of the four collection areas 35, 36, 37 and 38 is positioned below a corresponding one of the banks of synthesis columns on the cartridge 3. The drain plate 19 is rotated with the cartridge 3 to keep the corresponding collection area below the corresponding bank.

In FIG. 37, there are four drains 39, 40, 41, and 42 each of which is located within one of the four collection areas 35, 36, 37 and 38 respectively. In use, the collection areas are configured to contain material flushed from corresponding synthesis columns and pass that material through the drains. Preferably, there is a collection area and a drain corresponding to each bank of synthesis columns within the cartridge 3. Alternatively, any appropriate number of collection areas and drains can be included within a drain plate. FIG. 38A shows a top view of drain plate gaskets 43. The drain plate gasket is configured to be situated between drain plate 19 and cartridge 3. Drain plate gasket 43 is shown in FIG. 38A with guide holes 44 and drain cut-outs 57, 58, 59, and 60. Guide holes 44 allow the drain plate gasket to fit over the motor connector 22. Drain cut-outs 57-60 allow the bottom column opening of synthesis columns 12 to discharge material into collection areas 35-38 in drain plate 19. In other embodiments, the drain cut outs mirror the receiving holes in the cartridge (see cut-outs 60 in FIG. 38B), such that each column is able to discharge material into collection areas 35-38, while having a seal around each synthesis column. In some embodiments, all of the cut-outs are for the synthesis columns, like the cuts 60 depicted in FIG. 38B.

The drain plate gaskets of the present invention may be made of any suitable material (e.g. that will provide a tight seal above drain plate 19, such that gas and liquid do not escape). In some embodiments, the drain plate gasket is composed of rubber. Providing a tight seal between cartridge 3 and drain plate 19 with a drain plate gasket helps maintain the proper pressure of inert gas during purging procedures, such that synthesis columns with liquid reagent properly drain (preventing overflow during the next cycle). The seal between cartridge 3 and drain plate 19 may also be improved by the addition of grease between the components, or very finely machining the contact points between the two components. In other embodiments, the seal between the cartridge and drain plate is improved by physically bonding the plates together, or machining either the cartridge or drain plate such that concentric ring seals may inserted into the machined component. In still other embodiments, the two components are manufactured as a single component (e.g. a single components with all the features of both the cartridge and drain plate formed therein). In preferred embodiments, one component is provided with plurality of concentric circular rings that contact the flat surface of the other component and act as seals.

FIG. 39 shows a side view of a drain plate gasket 43 situated between cartridge 3 and drain plate 19. FIG. 39 also shows a drain 20 extending from drain plate 19. FIG. 39 also shows a drain with sealing ring 45 (sealing ring is labeled 46). The sealing ring 46 tightly seals the connection between the drain 45 and the waste tube system 23 (see FIG. 40). Also shown in FIG. 39 is a synthesis column 12 inserted in cartridge 3, passing through drain plate gasket 43, and ending in drain plate 19.

The waste tube system 23 is preferably utilized to provide a pressurized environment for flushing material including reagents from the plurality of synthesis columns located within a corresponding bank of synthesis columns and expelling this material from the synthesizer 1. Alternatively, the waste tube system 23 can be used to provide a vacuum for drawing material from the plurality of synthesis columns located within a corresponding bank of synthesis columns.

A cross-sectional view of the waste tube system 23 is illustrated in FIG. 39. The waste tube system 23 comprises a stationary tube 47 and a mobile waste tube 48. The stationary tube 47 and the mobile waste tube 48 are slidably coupled together. The stationary tube 47 is attached to the chamber bowl 18 and does not move relative to the chamber bowl (see FIG. 41). In contrast, the mobile tube 48 is capable of sliding relative to the stationary tube 47 and the chamber bowl 18. When in an inactive state, the waste tube system 47 does not expel any reagents. During the inactive state, both the stationary tube 47 and the mobile tube 48 are preferably mounted flush with the bottom portion of the chamber bowl 18 (see FIG. 41). When in an active state, the waste tube system 23 purges the material from the corresponding bank of synthesis columns. During the active state, the mobile tube 48 rises above the bottom portion of the chamber bowl 18 towards the drain plate 19. The drain plate 19 is rotated over to position a drain corresponding to the bank to be flushed, above the waste tube system 23. The mobile tube 48 then couples to the drain (e.g., 20 or 45) and the material is flushed out of the corresponding bank of synthesis columns and into the drain plate 19. The liquid reagent is purged from the corresponding bank of synthesis columns due to a sufficient pressure differential between a top opening 49 (FIG. 44) and a bottom opening 50 (FIG. 44) of each synthesis column. This sufficient pressure differential is preferably created by coupling the mobile waste tube 48 to the corresponding drain. Alternatively, the waste tube system 23 may also include a vacuum device 29 (see, FIG. 34) coupled to the stationary tube 47 (see FIG. 40) wherein the vacuum device 29 is configured to provide this sufficient pressure differential to expel material from the corresponding bank of synthesis columns. When this sufficient pressure differential is generated, the excess material within the-synthesis columns being flushed, then flows through the corresponding drain and is carried away via the waste tube system 23.

When engaging the corresponding drain to flush a bank of synthesis columns, preferably the mobile tube 48 slides over the corresponding drain such that the mobile tube 48 and the drain act as a single unit. Alternatively, the waste tube system 23 includes a mobile tube 48 which engages the corresponding drain by positioning itself directly below the drain and then sealing against the drain without sliding over the drain. The mobile tube 48 may include a drain seal positioned on top of the mobile tube. In this embodiment, during a flushing operation, the mobile tube 48 is not locked to the corresponding drain. In the event that this drain is accidentally rotated while the mobile waste tube 48 is engaged with the drain, the drain and mobile tube 48 of the synthesizer 1 will simply disengage and will not be damaged. If this occurs while material is being flushed from a bank of synthesis columns, any spillage from the drain is contained within the chamber bowl 18. In preferred embodiments, the bottom of the chamber bowl 18 has a chamber drain 64 (see FIG. 41) to collect and remove any spilled material in the chamber bowl. In this regard, material may be removed before it builds up and leaks into other parts of the synthesizer (e.g. motor 16 or gear box 17). In some embodiments of the present invention, the chamber drain is in a closed position during synthesis and purging. When the top cover of the synthesizer is opened, the chamber drain can be opened, drawing out unwanted gaseous or liquid emissions (e.g., using a vacuum source). Coordination of the chamber drain opening to the top cover opening may be accomplished by mechanical or electric means.

Configuring the waste tube system 23 to expel the reagent while the mobile waste tube 48 is coupled to the drain allows the present invention to selectively purge individual banks of synthesis columns. Instead of simultaneously purging all the synthesis columns within the synthesizer 1, the present invention selectively purges individual banks of synthesis columns such that only the synthesis columns within a selected bank or banks are purged. In preferred embodiments, the waste system is fitted for qualitative monitoring of detritylation. For example, colorimetric analysis of waste effluent using, for example, a CCD camera or a similar device provides a yes/no answer on a particular detritylation level. Qualitative analysis can also be accomplished by spectrophotometricly, or by testing effluent conductivity. Qualitative detection of detritylation can generally be performed with less expensive equipment than is generally required by more precise quantitation, and yet generally provides sufficient monitoring for detritylation failure. In preferred embodiments, the effluent from each column is monitored when a bank of columns is purged.

Preferably, the synthesizer 1 includes two waste tube systems 23 for flushing two banks of synthesis columns simultaneously. Alternatively, any appropriate number of waste tube systems can be included within the synthesizer 1 for selectively flushing synthesis columns or banks of synthesis columns. In preferred embodiments, the waste tube systems 23 are spaced on opposite sides of the chamber bowl 18 (i.e. they are directly across from each other, see FIG. 41). In this regard, the force on the drain plate 19 is equalized during flushing procedures (e.g. the drain plate is less likely to tip one way or the other from force being applied to just one side of the plate). Alternatively, a single waste tube system 23 may be provided for flushing the plurality of banks of synthesis columns. When a single waste tube system is used, it is preferred that a balancing force be provided on the opposite side of the drain plate 19, e.g., such as would be provided by the presence of a second waste tube system 23. In one embodiment, a balancing force is provided by a dummy waste tube system (not shown), that may be actuated in the same fashion as the waste tube system 23, but which does not serve to drain the bank of synthesis columns to which it is deployed.

In use, the controller 24, which is coupled to the motor 16, the valves 15, and the waste tube system 23, coordinates the operation of the synthesizer 1. The controller 24 controls the motor 16 such that the cartridge is rotated to align the correct synthesis columns with the dispense lines 6 corresponding to the appropriate valves 15 during dispensing operations and that the correct one of the drains 39, 40, 41, and 42 are aligned with an appropriate waste tube system 23 during a flushing operation.

In some preferred embodiments, the synthesizer comprises a means of delivering energy to the synthesis columns to, for example, increase nucleic acid coupling reaction speed and efficiency, allowing increased production capacity. In some embodiments, the delivery of energy comprises delivering heat to the chamber or the columns. In addition to increasing production capacity, the use of heat allows the use of alternate synthesis chemistries and methods, e.g., the phosphate triester method, which has the advantages of using more stable monomer reagents for synthesis, and of not using tetrazole or its derivatives as condensation catalysts. Heat may be provided by a number of means, including, but not limited to, resistance heaters, visible or infrared light, microwaves, Peltier devices, transfer from fluids or gasses (e.g., via channels or a jacketed system). In some embodiments, heat generated by another component of a synthesis or production facility system (e.g., during a waste neutralization step) is used to provide heat to the chamber or the columns. In other embodiments, heat is delivered through the use of one or more heated reagents. Delivery of heat also comprises embodiments wherein heat is created within the, e.g., by magnetic induction or microwave treatment. In some embodiments, heat is created at or within synthesis columns. It is contemplated that heating may be accomplished through a combination of two or more different means.

In some embodiments, the delivery of heat provides substantially uniform heating to two or more synthesis columns. In some embodiments, heating is carried out at a temperature in a range of about 20° C. to about 60° C. The present invention also provides methods for determining an optimum temperature for a particular coupling chemistry. For example, multiple synthesizers are run side-by-side with each machine run at a different temperature. Coupling efficiencies are measured and the optimum temperature for one or more incubations times are determined. In other embodiments, different amounts of heat are delivered to different synthesis columns within a single synthesizer, such that different reaction chemistries or protocols can be run at the same time.

Delivery of heat to an enclosed, sealed system will alter the pressure within the system. It is contemplated that the sealed system of the present invention will be configured to tolerate variations in the system pressure (i.e., the pressure within the sealed system) related to heating or other energy input to the system. In preferred embodiments, the system (e.g., every component of the system and every junction or seal within the system) will be configured to withstand a range of pressures, e.g., pressures ranging from 0 to at least 1 atm, or about 15 psi. It is contemplated that pressures may be varied between different points within the system. For example, in some embodiments, reagents and waste fluids are moved through the synthesis column by use of a pressure differential between one end (e.g., an input aperture) and the other (e.g., a drain aperture) of the synthesis column. In some embodiments, the system of the present invention is configured to use pressure differentials within a pressurized system (e.g., wherein a system segment having lower pressure than another system segment nonetheless has higher pressure than the environment outside the sealed system). In some embodiments, the prevention of backward flow of reagents through the system (e.g., in the event of back pressure from a process step such as heating) is controlled by use of pressure. In other embodiments, valves are provided to assist in control of the direction of flow.

In other preferred embodiments, the synthesizer comprises a mixing component configured to mix reaction components, e.g., to facilitate the penetration of reagents into the pores of the solid support. Mixing may be accomplished in a number of ways. In some embodiments, mixing is accomplished by forced movement of the fluid through the matrix (e.g., moving it back and forth or circulating it through the matrix using pressure and/or vacuum, or with a fluid oscillator). Mixing may also be accomplished by agitating the contents of the synthesis column (e.g., stirring, shaking, continuous or pulsed ultra or subsonic waves). Examples are provided in FIGS. 42A-C, which illustrate different embodiments of energy input components 95 and mixing components 96. Also, FIGS. 43A-B illustrate different combinations of energy input components 95 and mixing components 96.

In some preferred embodiments, an agitator is used that avoids the creation of standing waves in the reaction mixture. In some preferred embodiments, the agitator is configured to utilize a reaction vessel surface or reaction support surface (e.g., a surface of a synthesis column) to serve as resonant members to transfer energy into fluid within a reaction mixture. In a preferred embodiment, a horn is applied directly to the cartridge 3 to provided pulsed or continuous ultra sonic energy to the synthesis columns therein. In some embodiments, the matrix is an active component of the mixing system. For example, in some embodiments, the matrix comprises paramagnetic particles that may be moved through the use of magnets to facilitate mixing. In some embodiments, the matrix is an active component of both mixing and heating systems (e.g., paramagnetic particles may be agitated by magnetic control and heated by magnetic induction). It is contemplated that any of these mixing means may be used as the sole means of mixing, or that these mixing components may be used in combination, either simultaneously or in sequence. In preferred embodiments, the heating component and the mixing component are under automated control.

FIG. 42 illustrates a cross sectional view of a synthesis column 12. The synthesis column is an integral portion of the synthesizer 1. Generally, the polymer chain is formed within the synthesis column 12. More specifically, the synthesis column 12 holds a solid support 54 on which the polymer chain is grown. Examples of suitable solid supports include, but are not limited to, polystyrene, controlled pore glass, and silica glass. As stated previously, to create the polymer chain, the solid support 54 is sequentially submerged in various reagents for a predetermined amount of time. With each deposit of a reagent, an additional unit is added, or the solid support is washed, or failure sequences are capped, etc. Preferably, the solid support 54 is held within the synthesis column 12 by a bottom frit 55. In particularly preferred embodiments, a top frit 53 is included above the solid support (e.g. to help resist downward gas pressure when the particular synthesis column does not have liquid reagents, but other synthesis columns within the bank are being purged of their liquid contents). The synthesis column 12 includes a top opening 49 and a bottom opening 50. During the dispensing process, the synthesis column 12 is filled with a reagent through the top opening 49. During the purging process, the synthesis column 12 is drained of the reagent through the bottom opening 50. The bottom frit 55 prevents the solid support from being flushed away during the purging process.

The exterior surface 61 of each synthesis column 12 fits within the receiving hole 11 within the cartridge 3 and provides a pressure tight seal around each synthesis column within the cartridge 3. Preferably, each synthesis column is formed of polyethylene or other suitable material. In preferred embodiments, the receiving holes 11 of the cartridge 3 are provided with seals, such as O-ring seals 67, that will flex on engagement of the synthesis column 12 in receiving hole 11 and accommodate any irregularities in the exterior surface 61 of the synthesis column 12, thus assuring the presence of a pressure-tight seal.

In preferred embodiments, the material inside the synthesis column (e.g. in FIG. 44, this includes top frit 53, solid support 54, and bottom frit 55) is configured to resist the downward pressure of gas (e.g., to provide back pressure) applied during the purging process when the particular synthesis column does not have liquid reagent. In this regard, other synthesis columns that do contain liquid reagents may be successfully purged with the application of gas pressure during the purging process (i.e. the synthesis columns without liquid reagent do not allow a substantial portion the gas pressure applied during the purging process to escape through their bottom openings). Other packing materials may also be added to the synthesis columns to help maintain the pressure differential across the column when it is idle.

One method for constructing a synthesis column that successfully resists the downward pressure of gas (when no liquid reagent has been added to this column) is to include a top frit in addition to a bottom frit. Determining what type of top frit is suitable for any given synthesis column and type of solid support may be determined by test runs in the synthesizer. For example, the columns may be loaded into the synthesizer with the candidate top frit (and solid support and bottom frit), and instructions for synthesizing different length oligonucleotides inputted (i.e., this will allow certain columns to sit idle while other columns are still having liquid dispensed into them and purged out). Observation through the glass panel, examining the amount of leakage from overflowing columns, and testing the quality of the resulting oligonucleotides, are all methods to determine if the top frit is suitable (e.g., a thicker or smaller pore top frit may be employed if problems associated with insufficient back pressure are seen). By combining the appropriate packing material in columns with the appropriate delivered pressure to the chamber, purging can be efficiently carried out, avoiding spill-over that can result in synthesis or instrument failure.

Another method for constructing a synthesis column that successfully resists the downward pressure of gas (when no liquid reagent has been added to this column) is to provide a solid support that resists this downward force even when no liquid reagent is in the columns. One suitable solid support material is polystyrene (e.g. U.S. Pat. No. 5,935,527 to Andrus et al., hereby incorporated by reference). In some embodiments, the styrene (of the polystyrene) is cross-linked with a cross-linking material (e.g. divinylbenzene). In some embodiments, the cross-linking ratio is 10-60 percent. In preferred embodiments, the cross-linking ration is 20-50 percent. In particularly preferred embodiments, the cross-linking ratio is about 30-50 percent. In some embodiments, the polystyrene solid support is used in conjunction with a top frit in order to successfully resist the downward pressure of gas during the purging process. In some embodiments, the polystyrene is used as the solid support for synthesis. In other embodiments, a different support, such as controlled pore glass, is used as the support for the synthesis reaction, and the polystyrene is provided only to increase the back pressure from a column comprising a CPG or other synthesis support.

There are many advantages of configuring synthesis columns to successfully resist downward gas pressure during the purging process. One advantage is the fact that not all the synthesis columns need to contain liquid reagent during the purging process in order for the purge to be successful. Instead, one or more of the synthesis columns may remain idle during a particular cycle, while the other synthesis columns continue to receive liquid reagents. In this regard, oligonucleotides of different lengths may be constructed (e.g., a 20-mer constructed in one synthesis column may be completed and sit idle, while a 32-mer is constructed in a second synthesis column). Achieving successful purges after each liquid addition prevents liquid leakage (e.g. additional liquid reagent applied to a synthesis column that was not successfully purged will cause the column to overflow).

FIG. 45 illustrates a computer system 62 coupled to the synthesizer 11. The computer system 62 preferably provides the synthesizer 1, and specifically the controller 24, with operating instructions. These operating instructions may include, for example, rotating the cartridge 3 to a predetermined position, dispensing one of a plurality of reagents into selected synthesis columns through the valves 15 and dispense lines 6, flushing the first bank of synthesis columns 4 and/or the second bank of synthesis columns 5, and coordinating a timing sequence of these synthesizer functions. U.S. Pat. No. 5,865,224 to Ally et al. (herein incorporated by reference in its entirety), further demonstrates computer control of synthesis machines. Preferably, the computer system 62 allows a user to input data representing oligonucleotide sequences to form a polymer chain via a graphical user interface.

After a user inputs this data, the computer system 62 instructs the synthesizer 1 to perform appropriate functions without any further input from the user. The computer system 62 preferably includes a processor, an input device and a display. The computer 62 can be configured as a laptop or a desktop, and may be operably connected to a network (e.g. LAN, internet, etc.).

In some embodiments, the present invention provides alignment detectors for detecting the alignment of any of the components of the present invention, as desired. In some embodiments, when a misalignment is detected, an alarm or other signal is provided so that a user can assure proper alignment prior to further operation. In other embodiments, when a misalignment is detected, a processor operates a motor to adjust that alignment. Alignment detectors find particular use in the present invention for assuring the alignment of any components that are involved in an exchange of liquid materials. For example, alignment of dispense lines and synthesis columns and alignment of drains and waste tubes should be monitored. Likewise, the tilt angle of the cartridge or any other component that should be parallel to the work surface can be monitored with alignment detectors.

As noted above, the exterior surface 61 of each synthesis column 12 fits within the receiving hole 11 within the cartridge 3 and is intended to provide a pressure-tight seal around each synthesis column 12 within the cartridge 3. FIG. 46 illustrates three cross-sectional detailed views of the assembly 66 (the assembly comprising the cartridge 3, the drain plate gasket 43 and the drain plate 19) with a synthesis column 12 within a receiving hole 11 of cartridge 3. Each view shows a different embodiment of an airtight seal between the assembly 66 and the exterior surface 61 of synthesis column 12. In some embodiments, the airtight seal is provided by an O-ring 67. In preferred embodiments, the O-ring 67 is accessible for easy insertion and removal, e.g., for cleaning or replacement. In one embodiment, an O-ring 67 is positioned at the top of receiving hole 11, held in place by, e.g., a restraining plate 68, or any other suitable restraining fitting. In a preferred embodiment, a channel 69 is provided at the top of receiving hole 11 in cartridge 3 to accommodate the O-ring 67, as illustrated in FIG. 46A. In a particularly preferred embodiment, a groove 70 within receiving hole 11 in cartridge 3 accommodates an O-ring 67, providing a groove lip 71 to restrain the O-ring 67, as illustrated in FIG. 46B. In a particularly preferred embodiment, the groove lip 71 is about 0.030 inches. FIG. 46C illustrates a further embodiment, in which drain plate gasket 43 is configured to provide an airtight seal between nucleic acid synthesis column 12 and assembly 66. The illustrations in FIG. 46 are provided by way of examples only, and it is not intended that the present invention be limited by details of these illustrations, such as apparent size, shape or precise locations of features such as grooves, channels, plates or seals. Any O-ring configuration that helps maintain proper pressure differential across the synthesis columns is contemplated. O-rings 67 may be composed of any suitable material, preferably a chemically resistant, resilient material that flexes upon engagement of the synthesis column 12 in receiving hole 11. In some embodiments, a low cost material such as silicone or VITON may be used. In other embodiments, more expensive materials offering longer term stability, such as KALREZ, may be used. In some embodiments the O-rings may have a light lubrication, e.g. with a silicone or fluorinated grease.

In some embodiments, the present invention provides a means of collecting emissions from reagent reservoirs 72 (See e.g., FIGS. 47A and B) by providing a reagent dispensing station. In one embodiment, the reagent dispensing station is an integral part of the base 2 of the synthesizer, as illustrated in FIGS. 47A and 47B. In some embodiments, the reagent dispensing station provides an enclosure for collecting emitted gasses. In some embodiments, the enclosure is created by the provision of a panel 73 to enclose a portion of base 2 containing reagent reservoirs 72, as illustrated in FIG. 47B. In some embodiments, the panel 73 is movable for easy access to reagent reservoirs. In some embodiments, it is removeably attached. Removable attachment may be accomplished by any suitable means, such as through the use of VELCRO, screws, bolts, pins, magnets, temporary adhesives, and the like. In preferred embodiments, at least a portion of the panel 73 is slidably moveable. In preferred embodiments, at least a portion of panel 73 is transparent. In some embodiments, the enclosure of the reagent dispensing station comprises a viewing window that is not in a panel 73.

In some embodiments, the enclosure comprises a ventilation tube. In preferred embodiments, panel 73 comprises a ventilation port 74, e.g., for attachment to a ventilation tube. Since reagent vapors are typically heavier than air, in preferred embodiments, the ventilation tube is attached at the bottom for the enclosure. In a particularly preferred embodiment, the ventilation port is positioned toward the rear of the instrument.

In some embodiments, the enclosure further comprises an air inlet. In a preferred embodiment, a clearance 75 between the panel 73 and the base 2 provides an air inlet. In a particularly preferred embodiment, the air inlet is positioned toward the front of the instrument.

The location of the ventilation port 74 and air inlet is not limited to the panel 73. For example, in an alternative embodiment, the reagent dispensing station comprises a stand for holding the reagent bottles and a ventilation tube, wherein the stand holds the reagent reservoirs and the ventilation tube removes emitted gases.

Ventilation may be continuous or under the control of an operator. For example, in some embodiments, when the panel 73 is in a closed position, ventilation occurs continuously through the ventilation port 74 or at regular intervals. In other embodiments, an operator may manually activate ventilation prior to opening the panel 73. In still other embodiments, ventilation occurs in an automated fashion immediately prior to the opening of panel 73. For example, where the opening of panel 73 is controlled by a computer processor, activation of the “open” routine triggers ventilation prior to the physical opening of panel 73. In still other embodiments, the contents of the reagent containers are monitored by a sensor and the ventilation is triggered when one or more of the reagent containers are depleted. In some embodiments, the panel 73 is also automatically open, indicating the need for additional reagents and/or allowing an automated reagent container delivery system to supply reagents to the system.

The present invention also provides systems for ventilation, particularly ventilation of reaction enclosures (e.g., a chamber bowl 18), that improve the safety of synthesizers. The ventilation systems of the present invention may be applied to any type of synthesizer, and preferably, to open type synthesizers. These systems are particularly useful for improving the function and safety of certain commercially available synthesizers, such as the ABI 3900 Synthesizer.

During normal operations and without any malfunction, fumes are nonetheless are emitted from the chamber bowl of the 3900 machine when the synthesizer is opened for access by an instrument operator (e.g., when the top cover or lid enclosure is opened to retrieve columns after synthesis is completed). These emissions can be significant. In some instances, instruments such as the 3900 may be installed inside chemical fume hoods to collect such emissions from normal operations. However, placing machines in chemical fume hoods is not practical for a number of reasons: For example, the presence of a large instrument within a chemical fume hood limits the use of the hood for other purposes. Removal of the instrument when the hood is needed for another purpose is impractical, since many synthesizers are physically connected to external reagent reservoirs, gas tanks or other supply sources, making frequent removal and reinstallation prohibitively complex. Another problem with using chemical fume hoods to contain and remove emissions is that, using this approach, the number of synthesizers that can be used at one time is limited by the amount of hood space available. This prevents the use of many synthesizers in parallel, e.g., in an array of synthesizers, and therefore limits high-throughput synthesis capability. What is needed are systems to properly vent synthesizers, such as the 3900, that do not require placing the machines in chemical fume hoods.

The present invention provides systems for collecting emissions from synthesizers without the use of a separate fume hood. The present invention comprises a synthesizer having an integrated ventilation system to contain and remove vapor emissions. By way of example, the integrated ventilation system of the present invention is described as applied to the components and features of open synthesizers like the Applied Biosystems 3900 instrument. However, this configuration is used only as an example, and the integrated ventilation systems are not intended to be limited to the 3900 instrument or to any particular synthesizer. One aspect of the invention is to collect and remove vapors when the instrument is open, e.g., for access by the operator to the reaction chamber (FIGS. 48C, and 49A-C). In one embodiment of the present invention, the integrated ventilation system comprises a ventilated workspace. Embodiments of an integrated ventilation system comprising a ventilated workspace as applied to the 3900 instrument are shown in FIGS. 48A-C, 49A-C and 50A-B. Another embodiment is diagrammed in FIGS. 51A and B.

In some embodiments, a ventilation opening is provided through an opening in the top. For example, referring to FIG. 48A, in certain embodiments, some embodiments of synthesizers of the present invention comprise a top enclosure (e.g. 97) that forms a primarily enclosed space 104 over a top cover (e.g., 30, not shown in this figure). In preferred embodiments, the top enclosure has four sides (e.g., 98, two of which are shown in FIG. 48A), and a top panel (e.g., 99) that form a primarily enclosed space 104 above the top cover (e.g., 30) containing a plurality of valves (e.g., 10, not shown in this figure) and a plurality of dispense lines (e.g., 6, not shown in this figure). In certain embodiments, the top panel (e.g., 99) contains an outer window (e.g., 101). In some preferred embodiments, the outer window contains a ventilation opening (e.g., 105).

As used herein, the combination of a top enclosure (e.g., 97) and top cover (e.g., 30) is referred to collectively as the “lid enclosure” (e.g., 102). In preferred embodiments, the “lid enclosure” has six sides, with the top cover (e.g., 30) serving as the “bottom”, the top panel serving as the surface opposite the top cover, and the four side walls being the top enclosure sides (e.g., 98). In certain embodiments, the lid enclosure has a ventilation opening (e.g., 105) with a ventilation tube (e.g., 103) attached thereto (See, FIG. 48B). In preferred embodiments, the ventilation tube is connected to a ventilation opening in an outer window 101.

In other embodiments, the synthesizer base (e.g., 2) comprises a primarily enclosed space 104. In certain embodiments, a base (e.g., 2) of a synthesizer comprises a ventilation opening (e.g., 105) with a ventilation tube (e.g., 103) attached thereto (See, e.g., FIGS. 51A and 51B).

The ventilation openings in the lid enclosure or the base may be in any suitable position.. For example, the ventilation opening in the lid enclosure may be in the top panel (e.g. in the center, toward the back of the machine, or in one of the corners). The ventilation opening may also be located in a top enclosure side. For example, the ventilation opening may be in the enclosure side at the back of the machine, or on one of the sides (e.g., configured such that the lid enclosure may still be moved upward and downward while attached to a ventilation tube). A ventilation opening in a base may be, for example, on the front, the sides or on the back (e.g., configured such that the lid enclosure may still be moved upward and downward without interference by the ventilation tube). In preferred embodiments, the ventilation opening is positioned toward the rear (e.g., on a side or in the back) to allow the ventilation tubing to be directed away from an instrument operator. In particularly preferred embodiments, the ventilation opening is on the back of the base, e.g., as shown in FIGS. 51A and 51B.

In some embodiments, the ventilation is located in a position such that air traveling through the primarily enclosed space (e.g., 104) make greater or less contact with particular synthesizer components located inside the lid enclosure (e.g. valves, solenoids, dispense lines, etc.). The lid enclosures of the present invention may also have a plurality of ventilation openings. This may be desirable in order to control or direct air flow through the primarily enclosed space (e.g., to minimize or to maximize air contact with particular synthesizer components inside the lid enclosure).

As shown in FIG. 48C, in certain embodiments, the lid enclosure is hinged so that is may be moved upward and downward (e.g., allowing access to the chamber bowl or other reaction chamber by a user). In some embodiments, the primarily enclosed space of the lid enclosure (e.g. 104, not shown in this figure) is open to the ambient environment through a ventilation slot (e.g. 100) in the top cover or the top enclosure (e.g. in top enclosure side wall towards the back of the machine).

In certain embodiments of the present invention, a lid enclosure is present on a commercially available machine (e.g., ABI 3900), and the lid enclosure is modified as described herein (e.g., a ventilation opening is made in the lid enclosure) An opening near the hinge for wiring serves as a ventilation slot on the 3900. In other embodiments, the lid enclosure must be added to synthesizer. For example, a synthesizer that simply has a top cover (e.g., 30), may have a top enclosure (e.g., 97) added thereto. This may be done by attaching a top enclosure that has bottom flanges (opposite the top panel) that fit around the top cover, and provide a point of attachment (e.g., bolts, screws, adhesives, etc.). In other embodiments, the lid enclosure is fabricated as a separate component, then installed onto a synthesizer. For example, the components making up the lid enclosure (top enclosure and top cover) may be formed from a single mold, or two molds, etc. In this regard, features of the present invention may be built into the lid enclosure, such as the ventilation opening, ventilation slot, and certain hood components (described below).

In some embodiments, e.g., as diagrammed in FIGS. 48A-C, the lid enclosure (e.g., 102) comprises, or is modified to comprise at least one ventilation opening (e.g., 105). One or more ventilation openings may be used. In preferred embodiments, a ventilation opening is placed in the center of the top panel so as to avoid blocking the operator's view of internal components, such as the synthesis columns, during operation. In preferred embodiments, the lid enclosure comprises windows constructed of transparent or translucent material, such as plexiglass.

In preferred embodiments, the lid enclosures of the present invention comprise a top panel directly opposite a top cover, and side walls between these two components The primarily enclosed space between the top panel and top cover is, in some embodiments, open to the ambient environment through a ventilation slot near the lid enclosure hinge (e.g., 106). In certain embodiments, the lid enclosure of the present invention comprises an inner window and an outer window (e.g. an outer window in the top panel, and an inner window in the top cover). The outer window of the instrument allows visual inspection of operations and components within the lid and within the chamber bowl 18 of the base 2. The inner window seals the chamber bowl 18 by pressing against the chamber gasket when the lid enclosure is closed. Reagent supply tubing passes through the inner window, but the window is sealed around each tube so that the chamber will maintain appropriate pressure during operation. In the embodiment shown in FIG. 48B, the ventilation opening provides an aperture is the outer window.

In preferred embodiments, the ventilation opening (e.g., 105) is attached to a ventilation tube (e.g., 103), that in turn may be attached to an exhaust system. In some embodiments, a synthesizer is attached to an individual exhaust system. In other embodiments, multiple synthesizers are attached to a centralized exhaust system (e.g. centralized venting or vacuum system). In a preferred configuration, access to the exhaust system is toward the rear of the instrument, to minimize or prevent interference by the ventilation tubing with operator access to the chamber bowl, and to conduct the fumes away from instrument operators. The centralized exhaust may be a constant vacuum or a periodically actuated vacuum. In particular embodiments, raising the top cover or lid enclosure of a synthesizer triggers the vacuum system. In certain embodiments, reagent bottles on the sides of a synthesizer may also be vented through ventilation ports employing the same ventilation system employed by the ventilation tube attached to the top panel.

Another aspect of the present invention is to provide a ventilated workspace (e.g., around the chamber bowl) having a negative air pressure relative to the surrounding air pressure, such that the flow of air goes from the surrounding room into the ventilated workspace, and not in the reverse, during operation of the ventilation system (e.g., as shown in FIG. 50B and 50B). The ventilated workspace is designed to allow the instrument operator to reach into the space (e.g., to remove the synthesis columns) without turning off the ventilation system. One embodiment of a ventilated workspace is shown in FIG. 49A, wherein the ventilated workspace is created by providing side panels (e.g., 107). Two variations of another embodiment are shown in FIGS. 49B and 49C. In this embodiment, the ventilated workspace is created by providing side panels (e.g., 107) between the body of the synthesizer and the lid enclosure, and a front panel (e.g., 108). In certain embodiments, the ventilated workspace is created by including only side panels. In other embodiments, the ventilated workspace is created by only including a front panel. In preferred embodiments, side and front panels are used together (e.g., as in FIGS. 49B and 49C) to create a ventilated workspace. In some embodiments, side and front panels are provided as separate components. In other embodiments, a single component comprising both side panels and a front panel is provided.

The size of the ventilated workspace can be altered by the placement of the panels, e.g., the side panels (107) shown in FIGS. 49A-C. In some embodiments, panels are positioned to maximize the size of the enclosed ventilated workspace (e.g., as in FIG. 49B). In other embodiments, the panels are positioned to provide a smaller ventilated workspace (e.g., as with the side panels in FIG. 49C). In some preferred embodiments, the side panels are positioned as close to the top chamber gasket (e.g., 31) as they can be without disturbing the seal between the top chamber gasket and the top cover 30. In certain embodiments, the front and/or side panels are used with a synthesizer only having a top cover (not a full lid enclosure).

The side panels can be made of a number of different materials. In some embodiments, the materials used for the side panels are opaque. In other embodiments, the side panels are translucent or clear (e.g., to permit surrounding light into the ventilated workspace). In certain embodiments, the side panels are constructed from flexible polymeric material (e.g., sheeting), such as polyethylene or polypropylene. In some embodiments, the polymeric material has an average thickness of about 2 to 8 mils. In preferred embodiments, the polymeric material has an average thickness of about 2 to 4 mils. In some embodiments, the panels are collapsible (i.e., can collapse or fold down upon themselves as the lid enclosure or top cover, is lowered). In some embodiments, panels are accordion-style or fan-fold style barriers that fold down upon themselves when the top cover or lid enclosure is lowered. In preferred embodiments, when the panels are collapsed, they have a total thickness that is less than the height of the O-ring or gasket (e.g., top chamber seal 31) on the interior of the synthesizer (e.g., so that there is no interference with the sealing of the O-ring).

In other embodiments, the side panels are constructed of rigid material. In some embodiments, rigid side panels are configured to fit into recesses in the body of the synthesizer when the top cover or lid enclosure is closed. In other embodiments, rigid side panels are configured to fit around the outside of the base of the synthesizer when the top cover or lid enclosure is closed. In some embodiments, rigid side panels are constructed from opaque materials (e.g., steel, aluminum, opaque plastic). In other embodiments, rigid side panels are constructed from translucent or transparent material, such as plexiglass. Generally, the side panels are connected to the top cover, so when the top cover or lid enclosure is raised, the side panels slide up to form sides for the ventilated workspace.

In certain embodiments, a front panel (e.g., 108) is attached to the lid enclosure. For example, the front panel may attach to the top cover (e.g., FIG. 49B), or the front panel may attach to one of sides of the lid enclosure (e.g., FIG. 49C). The front panel may drape over the front of the synthesizer when the lid enclosure is closed (See, e.g., FIGS. 48B and 49C). Alternatively, the front panel may fit into a recessed slot in the synthesizer base, or fold up upon itself as the lid enclosure is lowered into the closed position.

Attachment of the panels provided for the purpose of enclosing the ventilated workspace is not limited to any particular means. For example, in a simple configuration, panels are attached by use of strips of VELCRO fastener (e.g., adhesive backed strips), for easy mounting and removal. For a sturdier attachment, the panels may be attached using fasteners, including but not limited to screws, bolts, welds, and snaps, or may be attached with removable or permanent adhesives. The presence of the panels reduces the size of the opening through which ambient air can enter the ventilated workspace, and also reduces the size of the opening from which air and vapors in the chamber bowl can escape. When the ventilation system is turned on (e.g., when the connected ventilation tube is drawing air from the ventilation opening, the airflow through the reduced opening prevents or reduces any flow (e.g. outward flow) of gaseous emissions. When the ventilation system is actuated, ambient air and reagent vapors are drawn across the chamber bowl (e.g., 18) and into the ventilation slot (e.g., 100), as diagrammed in FIGS. 50B and 51B. The air and vapors then move through the primarily enclosed space (e.g., 104) and exit through the ventilation opening (e.g., 105) into the ventilation tube (e.g., 103). In some embodiments, the air flow rate at the opening of the ventilated workspace (e.g., in the embodiments shown in FIGS. 49B and 49C, where the surrounding air is drawn into the ventilated workspace below the front panel and between the side panels) is from about 20 to about 100 feet per minute, face velocity. In some preferred embodiments, the flow rate at the opening is about 40 to 5.0 feet per minute, face velocity.

From the ventilation tube, the air and vapors may be vented, treated or collected. In certain embodiments, the vented air and vapors are routed to a central scrubber. The central scrubber may form part of an overall emission control system. The central system may also be used to adjust total airflow for the number of synthesizers that are open at the same time. In this regard, exhaust from the system is minimized so as to concentrate waste vapors.

In order to increase or decrease the speed at which air and vapors travels through the ventilation system of the present invention, the size of the ventilation slot may be adjusted (e.g. reducing the size of the ventilation slot increase the speed of the moving air and vapors). The airflow pattern made possible by the present invention allows synthesizers to be opened (e.g. to change columns, etc) without exposure of an operator to hazardous vapors (e.g. argon, solvent fumes, etc).

The integrated chamber ventilation system of the present invention may be adapted to many synthesizers of both ‘open’ and ‘closed’ design. On example of another synthesizer that can be modified to include the reaction enclosure ventilation system of the present invention is the POLYPLEX 96-channel, high-throughput oligonucleotide synthesizer from GeneMachines, San Carlos, Calif., which comprises a synthesis case providing an enclosure for the synthesis block in which the reactions are performed. A similar instrument is described in WO 00/56445, published Sep. 28, 2000, and in related U.S. Provisional Patent application 60/125262, filed Mar. 19, 1999, each incorporated herein in their entireties. As described in WO 00/56445, the synthesis case has a loading station, drain station, and water-tolerant and water-sensitive reagent filling stations. The synthesis case has a cover, a first and a second side, a first and a second end, and a bottom side, which contacts the base. The load station comprises a sealable opening in the synthesis case through which a multiwell plate can be inserted. In application of the present invention, the synthesis case can be fitted with one or more ventilation openings similar to ventilation opening 105, for attachment to ventilation tubing (e.g., 103). In some embodiments, a ventilation opening is in a side of the synthesis case opposite the side having the sealable opening. In preferred embodiments, a ventilation opening in the synthesis case is on the first or second end. In particularly preferred embodiments, the ventilation system is actuated when the sealable opening is opened, e.g., for insertion or removal of a multiwell plate.

The present invention also contemplates robotic means (e.g. conveyor belt, robots, etc) for linking the synthesizers to other components of the production process. For example, FIG. 52 illustrates a synthesizer 1, a robotic means 92, a cleave and deprotect component 93 and a purification component 94 operably linked together.

The present invention provides synthesizer arrays (e.g., groups of synthesizers). In some embodiments, the synthesizers are arranged in banks. For example, a given bank of synthesizers may be used to produce one set of oligonucleotides. The present invention is not limited to any one synthesizer. Indeed, a variety of synthesizers are contemplated, including, but not limited to the synthesizers of the present invention, MOSS EXPEDITE 16-channel DNA synthesizers (PE Biosystems, Foster City, Calif.), OligoPilot (Amersham Pharmacia,), and the 3900 and 3948 48-Channel DNA synthesizers (PE Biosystems, Foster City, Calif.). In some embodiments, synthesizers are modified or are wholly fabricated to meet physical or performance specifications particularly preferred for use in the synthesis component of the present invention. In some embodiments, two or more different DNA synthesizers are combined in one bank in order to optimize the quantities of different oligonucleotides needed. This allows for the rapid synthesis (e.g., in less than 4 hours) of an entire set of oligonucleotides (all the oligonucleotide components needed for a particular assay, e.g., for detection of one SNP using an INVADER assay [Third Wave Technologies, Madison, Wis.]).

In some embodiments the DNA synthesizer component includes at least 100 synthesizers. In other embodiments, the DNA synthesizer component includes at least 200 synthesizers. In still other embodiments, the DNA synthesizer component includes at least 250 synthesizers. In some embodiments, the DNA synthesizers are run 24 hours a day.

### Synthesizer Example 1

### The Northwest Engineering 48-Column Oligonucleotide Synthesizer

The Northwest Engineering 48-Column Oligonucleotide Synthesizer (NEI-48, Northwest Engineering, Inc., Alameda, Calif.) is an “open system” synthesizer in that the dispensing tubes for the delivery of reagents are not affixed to each synthesis vial or column for the entire term of the synthesis process. Instead, movement of a round cartridge containing the columns allows each dispensing tube to serve multiple columns. In addition, when a synthesis column is positioned to receive reagent, the dispenser is not even temporarily affixed to the vial with a sealed coupling. The reagent dispensed to the vial has open contact with the surrounding environment of the chamber. The chamber containing the synthesis vials is isolated from the ambient environment by a top plate. The general design and operation of the NEI instrument is described in WO 99/656602.

The NEI-48 synthesizer includes external mounting points for various reagent bottles, such as the phosphoramidite monomers used to form the polymer chain, and the oxidizers, capping reagents and deblocking reagents used in the reaction steps. TEFLON tubing feeds liquid from each reagent bottle to its assigned valve on the top of the machine. The feeding is done under pressure from an argon gas source.

The operations of the machine are controlled using a computer. The computer is fitted with a motion control card connected via cabling to a motor controller in the synthesizer; in addition, the computer is connected to the synthesizer via an RS-232C cable. The provided software allows the user to monitor and control the machine's synthesis operations.

The machine also requires connection to a source of argon gas, to be delivered at a pressure between 15 and 60 psi, inclusive, and a source of compressed air or nitrogen, to be delivered at a pressure between 60 and 120 psi, inclusive.

Synthesis in the NEI-48 occurs within synthesizer columns that are arranged in the cartridge.

Operations of the NEI-48 in accordance with the manufacturer's instructions produced undesirable emissions and leakage resulting in potential synthesis and instrument failure. The following section details two of the sources of these emissions, and details one or more aspects of the present invention applied to solve each problem, to thereby improve the performance of this machine.

A. Column Overflow Due to Inadequate Argon Pressure

Undesirable emissions and exposure are increased when columns overflow, causing the hazardous reagents used during synthesis to collect in the chamber bowl. A number of types of malfunction in the machine can leads to incomplete drainage or purge of the columns, and each will eventually lead to column overflow as the instrument proceeds through its subsequent dispensing steps.

The flow of reagent and waste from the synthesis columns is controlled by a differential in the pressure of argon between the top and bottom openings of the column. When the pressure of argon on the top opening is not sufficiently high, the column will not drain or be purged completely, i.e., fluid that should be drained will remain in the column. This improper purging not only reduces the efficiency of the synthesis chemistry, it also leads to column overflow. Therefore, failure of either initial pressurization of the chamber, or leakage of argon from any coupling (in an amount great enough to reduce either the overall pressure of the system or the pressure differential across the synthesis column) may lead to undesirable emissions and exposure. One aspect of the present invention is to prevent column overflow by reducing leakage of argon at a variety of points in the system.

The NEI-48 demonstrated a variety of failures as a result of argon leakage from or within the instrument. To address this problem, the drain plate gasket 43 of the present invention was created and was fitted between the cartridge and drain plate. Addition of the gasket to this assembly, as diagramed in FIG. 38, provided a pressure-tight seal, thereby containing the argon and allowing proper drainage of the columns at the purging step. The gasket of the present invention applied in this way improved the safety of the machine, and improved the efficiency of the synthesis reaction.

In another embodiment, a modified drain plate gasket was provided. The drain plate has securing holes 33, for attachment of the motor connector 22. The first gasket was of a design that avoided the areas of the motor connector 22 and the securing holes 33. A modified drain plate gasket was designed with guide holes 44 to fit closely around each securing hole 33, such that the holes served to place the gasket in a specific position between the cartridge and the drain plate (FIG. 38). In an alternative embodiment, the drain plate 19 and the cartridge 3 may be provided with other alignment features, such as pin fittings and corresponding pin receiving holes (not shown) to facilitate alignment of these parts during assembly (e.g., after cleaning). A modified drain plate gasket for use with these parts may be provided with pin guide holes (not shown). Use of either the securing holes 33, or pins fittings to align the gasket makes the gasket easier to position during assembly, ensuring proper operation of the gasket and improving ease of any maintenance that requires disassembly of these parts.

B. Emissions from reagent bottles

During normal operations and without any malfunction, fumes can nonetheless be emitted by the reagent bottles attached to the machine. These emissions can be increased by poor fit or incorrect seals around bottle caps. For example, the reagent bottles for the NEI-48 are. affixed to the machine by clamps that apply pressure to the outside of the bottle caps. The clamps can distort the caps, increasing leakage and gaseous emissions.

One aspect of the present invention is to provide a means of collecting emissions from reagent bottles. For improving the NEI-48, a reagent stand comprising a ventilation tube was constructed. The stand holds the reagent bottles, thereby eliminating the need for the cap-distorting clamps, and consequently reducing emissions from the bottles; the ventilation tube removes any remaining emitted gases. This reagent dispensing station improves the safety of the machine in normal operation. The reagent dispensing station of the present invention is not limited to a configuration comprising a stand. It is envisioned that a station comprising a ventilation system may also be used with one or more bottles held in clamps. In preferred embodiments, at least one aspect of the reagent container system, e.g., the clamp, the cap, or the bottle, is modified such that clamping the reagent bottle does not compromise the containment function of the cap, or of any other aspect of the reagent container system.

### Synthesizer Example 2

### The Applied Biosystems 3900 Oligonucleotide Synthesizer

The Applied Biosystems 3900 Oligonucleotide Synthesizer (Applied Biosystems, Foster City, Calif.) is similar in design and function to the NEI-48, described above. The 3900 is an “open system” synthesizer utilizing a round cartridge containing the columns. The receiving holes of the cartridge are essentially cylindrical, and, as with the NEI-48, proper function of the instrument relies on an airtight seal between the columns and cartridge.

The 3900 synthesizer includes recessed areas for the external mounting of reagent bottles. When mounted on the instrument, the reagent bottles do not protrude beyond the outside edges of the instrument; they are completely recessed, (as, e.g., the reagent reservoirs 72 are recessed in base 2, diagrammed in FIG. 47A). As with the NEI-48, the reagent feeding is done under pressure from an argon gas source.

The performance of the 3900 synthesizer is improved using the modifications provided by the present invention. Two specific improvements are described below. These particular improvements are described by way of example; improvements to the ABI 3900 synthesizer, or any synthesizer, are not limited to the improvements described herein below.

A. Column Overflow Due to Inadequate Argon Pressure

As described above for the NEI-48, the proper purging of the synthesis columns at each cycle relies on the maintenance of a differential in argon pressure between the top and bottom openings of the columns. Improper or incomplete purging reduces the efficiency of the synthesis and increases the risk of column overflow. Proper purging in the 3900, like other open systems, depends in part upon the formation of an airtight seal between receiving holes in the cartridge and exterior surfaces of the synthesis columns. The presence of irregularities in the column shape or surface can prevent the formation of an airtight seal, allowing argon to leak around the column exterior, thereby disrupting the pressure differential required to properly purge the columns at each cycle. The need to discard columns having even minor imperfections adds expense to the use of the instrument. If undetected, a faulty seal can lead to poor synthesis and column overflow, as described above.

As discussed above, in some embodiments, the present invention provides improved synthesizers having reliable seals between the cartridge and the synthesis columns. The present invention provides a number of embodiments of synthesizers having such seals. For example, as described above, a synthesizer may be improved by the addition of a resilient seal, such as an O-ring, in the receiving hole of each cartridge.

To make this improvement, the 3900 is fitted with such O-rings for safer, more reliable and more efficient performance. Examples of several means of creating an improved seal between the outer surface of a column 61 and a receiving hole 11 are diagrammed in FIGS. 46A-46C. While any of the embodiments of seals disclosed herein may be applied to the 3900 instrument, in a preferred embodiment, the 3900 is improved by the use of an embodiment similar to that diagrammed in FIG. 46B, wherein a groove 70 creates a groove lip 71, to accommodate and hold an O-ring 67, thus providing a seal between cartridge 3 and the exterior surface 61 of the synthesis column 12. In a particularly preferred embodiment, the receiving hole 11 is enlarged in diameter to facilitate insertion and removal of an O-ring 67, e.g., for easy cleaning or replacement. A groove is machined into the interior of each receiving hole in a 3900 cartridge, and appropriate O-ring seals are placed in the grooves. As noted above, the O-ring could be of any suitable material. Thus modified, the cartridge of the 3900 has a greatly improved ability to accommodate imperfections in the exteriors of synthesis columns, and this improvement results in safer, and more efficient and reliable operation of the instrument, with fewer costs associated with chemical spill clean-up, instrument down-time, and the disposal of unusable synthesis columns.

B. Emissions from Reagent Bottles

During normal operations and without any malfunction, fumes are nonetheless emitted by the reagent bottles attached to the 3900 machine. These emissions can be significant, even though gaskets are provided for use in conjunction with the bottle caps.

As described above, the present invention provides a means of collecting emissions from reagent bottles. On the 3900, the reagent bottles are attached in recessed areas on the exterior in the base of the instrument (e.g., the reagent reservoirs 72 attached to the recessed areas in the base 2, as illustrated in FIG. 47A). The emissions from this instrument are reduced by modification to provide the enclosed reagent dispensing station of the present invention. In modification of the 3900, the recessed areas are provided with panels to enclose the space, reducing the release of hazardous vapors.

Reagent bottles or reservoirs need to be accessible for changing or filling, due, e.g., to consumption of reagents during synthesis operations. In making the modification to the 3900, the panels added to the instrument are moveable, to provide access to the reagent bottles within the enclosed space. In a simple configuration, panels provided for the purpose of enclosing the space are attached by use of strips of VELCRO fastener (e.g., adhesive backed strips), for easy mounting and removal. For a sturdier attachment, the panels may be attached using hard, removable fasteners, such as screws or bolts. In a particularly preferred configuration, the panels are mounted in tracks, brackets or other suitable fittings that allow them to be moved or removed by sliding.

To monitor reagent bottles (e.g., to determine when changing or filling is needed), it is, preferred that the reagent reservoirs be accessible for visual inspection. In making the addition of panels to the 3900, the panels are constructed such that the reagent bottles can be visually inspected without opening the enclosure. The panels provided are constructed of transparent material. While glass may be used, in preferred embodiments, for both safety and ease of handling a plastic is used with sufficient transparency to allow visual inspection of reagent bottles, and with sufficient resistance to the chemicals used in synthesis to avoid rapid or immediate decay or fogging, (as is often associated with exposure of plastics to vapors of solvents to which they are not resistant), when used in this application. Selection of plastics for appropriate chemical resistance is well known in the art, and tables of chemical compatibility are generally readily available from manufacturers.

The panels are provided with a ventilation port (e.g., ventilation port 74, as diagrammed in FIG. 47B), for the removal vapors and fumes emitted by the reagent bottles. Such a ventilation port serves as an attachment point for a ventilation tube to conduct fumes away from the instrument, e.g., into an exhaust system. Since the vapors from DNA synthesis reagents tend to be heavier than air, the ventilation port is placed near the bottom of the enclosure. Placement of the ventilation port toward the rear is convenient for attachment to a larger exhaust system, minimizes or prevents interference by the ventilation tubing with operator access to other parts of the instrument, and conducts the fumes away from instrument operators.

To maximize efficacy of the ventilation system, an air inlet into the enclosure is provided. In applying the panels to the 3900, a clearance between the attached panels and the body of the instrument (e.g., the clearance 75 between the panel 73 and the base 2 diagrammed in FIG. 47B) provides the air inlet. The panel is positioned such that the principal air inlet is a clearance between the front edge of the panel (i.e., the edge closest to the front of the instrument) and the instrument base. Positioning of the inlet toward the front of the instrument, or on the opposite side of an enclosure from a ventilation port, maximizes the flow of air through the enclosure, providing the most efficient removal of vapors. The inward flow of air minimizes the possible escape of hazardous vapors toward instrument operators. Thus modified, the 3900 instrument is improved with respect to its emissions of hazardous vapors.

C. Emissions from the Chamber Bowl

During normal operations and without any malfunction, fumes are nonetheless emitted when the chamber bowl of the ABI 3900 is opened for access by the instrument operator (e.g., when the lid is opened to retrieve columns after synthesis is completed). These emissions can be significant. The present invention provides a means of collecting emissions from the 3900 without the use of a separate fume hood. The present invention comprises a synthesizer having an integrated ventilation system to contain and remove vapor emissions. One aspect of the invention is to collect and remove vapors when the instrument is open. Embodiments of integrated ventilation systems as applied to the 3900 instrument are shown in FIGS. 48-51.

As shown in FIG. 48A, in one embodiment, the lid enclosure 102 is modified to comprise a ventilation opening 105. The lid enclosure of the 3900 comprises an outer window 101. In preferred embodiments, a ventilation opening is placed in the center of the outer window 101 of the lid enclosure 105, so as to avoid blocking the operator's view of internal components, such as the synthesis columns, during operation.

As shown in the diagram of FIG. 50, the lid enclosure of the 3900 instrument comprises an outer window 101 and an inner window 25. The space between the windows is open to the ambient environment through a ventilation slot 100 near the lid enclosure hinge 106. The outer window in an unmodified instrument allows visual inspection of operations and components within the lid enclosure and within the chamber bowl 18 of the base 2. Reagent supply tubing passes through the inner window, but the window is sealed around each tube so that the chamber will maintain appropriate pressure during operation. In the embodiment shown in FIGS. 48, 49 and 50, the ventilation opening provides an aperture in the outer window.

In another embodiment, one or more ventilation openings may be provided in the base (e.g., 2) of the synthesizer, as diagrammed in FIG. 51. In other embodiments, a synthesizer may comprise ventilation openings in both a lid enclosure and a base.

Each ventilation opening is attached to ventilation tubing (e.g., 103) for attachment to an exhaust system. In some embodiments, a synthesizer is attached to an individual exhaust system. In other embodiments, multiple synthesizers are attached to a centralized exhaust system. In a preferred configuration, the access to the exhaust system is toward the rear of the instrument, to minimize or prevent interference by the ventilation tubing with operator access to the chamber bowl, and to conduct the fumes away from instrument operators.

Another aspect of the present invention is to provide a ventilated workspace around the chamber bowl having a negative air pressure relative to the surrounding air pressure, such that the flow of air goes from the surrounding room into the ventilated workspace, and not in the reverse, during operation of the ventilation system. The ventilated workspace is designed to allow the instrument operator to reach into the space (e.g., to remove the synthesis columns) without turning off the ventilation system. Embodiments of a ventilated workspace are shown in FIG. 49A-C. As shown in this embodiment, the ventilated workspace is created by providing side panels between the body of the synthesizer and the lid enclosure, and a front panel. The presence of the panels reduces the size of the opening through which ambient air can enter the ventilated workspace. When the ventilation system is turned on (i.e., when the connected ventilation tube is drawing air from the ventilation opening, the airflow in through the reduced opening prevents or reduces any outward flow of gaseous emissions.

B. Closed System Synthesizers

In preferred embodiments, the present invention provides closed-system solid phase synthesizers that are suitable for use in large-scale polymer production facilities. Each synthesizer is itself capable of producing large volumes of polymers. Furthermore, the present invention provides systems for integrating multiple synthesizers into a production facility, to further increase production capabilities.

Currently available nucleic acid synthesizers have limited synthesis capacity. For example, the 3900 DNA Synthesizer (Applied Biosystem, Foster City, Calif.) is one of the most capable synthesizers and produces fewer than 100 40-mer oligonucleotides in a typical day production run. Additional synthesizers are described in U.S. Pat. Nos. 5,744,102, 4,598,049, 5,202,418, 5,338,831, 5,342,585, 6,045,755, and 6,121,054, and PCT publication WO 01/41918, herein incorporated by reference in their entireties.

The synthesizers of the present invention dramatically increase capacity, with some embodiments allowing over 2000 40-mer oligonucleotides to be produced per day (e.g., during a 16 hour production day) at a 1 μM scale. These capacities are achieved through the use of multi-chamber reaction supports that allow parallel synthesis of polymers within each chamber. For example, three or more chambers (e.g., comprising synthesis columns), preferably 96 or more chambers are provided on a reaction support, permitting a plurality of different oligonucleotides to be simultaneously produced. Each reaction chamber is associated with its own reagent dispenser such that reagents are delivered to each chamber substantially simultaneously rather than delivery reagents in sequence. In preferred embodiments, the synthesizer is a closed system during operation (i.e., reagent delivery to the chambers and waste removal from the chambers occurs in a continuous pathway that is isolated from the ambient environment). An example of a closed system is illustrated in FIG. 53. In some preferred embodiments, the synthesizers have a minimum number of moving parts. In particular, the reaction support is immobile.

In some embodiments, the synthesizer provides additional polymer production capabilities. For example, in some embodiments, the synthesizer is configured to conduct cleavage and deprotection of synthesized oligonucleotide. In preferred embodiments, the same reaction support is used for both synthesis and cleavage and deprotection. In other preferred embodiments, the same reagent dispensers are used for both synthesis and cleavage and deprotection. In still other preferred embodiments, the reaction support does not move during both the synthesis and cleavage and deprotection processes (i.e., synthesis and cleavage and deprotection occur at the same location). In some embodiments, the synthesizer also provides an integrated purification component (e.g., using the same reaction support and/or reagent dispensers with or without movement of the reaction support). Any other production components described herein may also be integrated with the synthesizer.

Preferred features of the synthesizers of the present invention include: single day synthesis capacities of 2000 oligonucleotides, based on an average 40-mer at 1 μM scale with 16 hours staffing; production scale capabilities of 40, 100, 1000, and 4000 nM, with larger scales supported by control elements; compatibility with commercially available nucleic acid synthesis columns (e.g., columns designed for use with EXPEDITE nucleic acid synthesizers [Applied Biosystems, Foster City, Calif.], 3900 High-Throughput Columns for use with the 3900 DNA Synthesizer [Applied Biosystems], DNA synthesis columns from Biosearch Technologies, Novato, Calif.); mechanical and/or data interface capability with other production components (see Section II, below); individual oligonucleotide tracking (e.g., during synthesis and throughout an entire production process); compatibility with standard nucleic acid synthesis chemistry with provisions for optimization of reaction conditions; detectors for monitoring trityl or other components or reagents; compatibility with standard multi-chamber formats (e.g., 96-well plate, 384-well plate formats); interface with databases to input and track information including, but not limited to oligonucleotide sequence, completion, data, time, and channel; and integration with a control system to allow multiple synthesizers to have a common control center.

Reagent delivery to the synthesizer is achieved using a novel fluidics system. In preferred embodiments, all fluid transfers are desired to be closed system; that is, a closed fluid circuit exists from source to waste at any time reagents are being transferred. In general, the supply circuit remains coupled to the synthesis columns that are supported by the reaction support for all operations except, in some embodiments, during nucleic acid coupling reactions. Given the reaction time required for the coupling reactions (approximately 30 seconds), in some embodiments, the circuit to a particular column or columns is disconnected to allow fluid transfer mechanisms to be used on other columns. While the fluid transfer is re-routed, the columns undergoing the coupling reaction need not be exposed to the ambient environment (i.e., a sealed delivery path may be maintained).

In preferred embodiments, the target fluid transfer system is a pressurized supply with dispense control valves. Reagents flow to the reaction chambers upon opening of the control valves, driven by a pressure differential.

In some preferred embodiments, the reaction support contains waste channels configured to receive waste from the reaction chambers. In some embodiments, each channel is configured with its own waste channel (See e.g., FIG. 53). The waste channels preferably feed into a single waste disposal line. In some embodiments, the waste system is gravity driven. In other embodiments, a valve-controlled vacuum is used to eliminate waste. In some preferred embodiments, waste lines are fitted with a trityl monitoring device. In preferred embodiments, the waste line is fitted with a qualitative trityl monitoring device. For example, calorimetric analysis of effluent using a CCD camera or a similar device provides a yes/no answer on a particular detritylation level. Qualitative detection of detritylation can generally be performed with less expensive equipment than is generally required by more precise quantitation, and yet generally provides sufficient monitoring for detritylation failure. Valves used to control reagent delivery and/or waste removal may be under automated control.

In preferred embodiments, a plurality of reagent dispensers are provided, wherein a reagent dispenser is provided for each reaction chamber. In such embodiments, the reagent dispensers provide each of the reagents necessary to support a synthesis reaction within the reaction chamber. For nucleic acid synthesis, this includes, for example, delivery of acetonitrile, phosphoramidite corresponding to each of the bases, argon gas, oxidizer, activator (e.g., tetrazole), deblocking solution and capping solution. Thus, in some embodiments, the reagent dispenser comprises a plurality of reagent delivery lines, each line providing a direct fluidic connection between the reagent dispenser and individual supply tanks for the different reagents (See e.g., FIG. 53).

An example of such a reagent dispenser (2) is shown in FIG. 54 from both a side view (FIG. 54A) and a cross-sectional bottom view (FIG. 54B). The side view shows a single reagent delivery line (3) penetrating a top surface (4) of the reagent dispenser (2). In this embodiment, a retention ring (5) is used to support the reagent delivery line (3). The reagent delivery line (3) ends at a reagent reservoir (6) that is configured to receive reagents from each of the delivery lines. A seal (7) forms a contact between the delivery line (3) and the reagent reservoir (6). The center of the reagent reservoir (6) comprises a delivery aperture (8). The delivery aperture (8) is in fluidic contact with a delivery channel (9), with a seal (10) forming a contact between the delivery channel (9) and the delivery aperture (8). The delivery channel (9) passes through a bottom surface (11) of the reagent dispenser (2) and may positioned by a retention ring (12).

The cross-sectional bottom view shown in FIG. 54B shows the presence of nine delivery lines (3) contained within the reagent dispenser (2). Each delivery line empties into the reagent reservoir (6), represented by the eight pronged star. FIG. 55A shows one preferred embodiment of the reagent dispenser (2), wherein the outer surface of the delivery channel (9) contains first (13) and second (14) ring seals configured to form an airtight or substantially airtight seal with one or more points on the interior surface of a synthesis column (15) or other reaction chamber (e.g., with reaction chambers present in a synthesizer or a cleavage and deprotection component; see, for example FIG. 55B).

In preferred embodiments, common reagent tanks supply reagents to all of the reaction chambers. The reagents tanks may be contained within the synthesizer or may be external to the synthesizer. Where the tanks are provided with the synthesizer, they are preferably contained in a vented chamber to reduce the build-up of gaseous or liquid waste in and around the synthesizer. In some preferred embodiments, common reagent tanks supply reagents to a plurality of synthesizers. Examples of such delivery systems are provided, below. In yet other embodiments, some of the reagents are supplied externally and some of the reagents are supplied at or in the synthesizer (e.g., amidites). In some embodiments, one or more of the reagents are processed, e.g., under vacuum, to remove dissolved gasses.

In some preferred embodiments, the synthesizer comprises a means of delivering energy to the reaction chambers to, for example, increase nucleic acid coupling reaction speed and efficiency, allowing increased production capacity. In some embodiments, the delivery of energy comprises delivering heat to the reaction chambers. In addition to increasing production capacity, the use of heat allows the:use of alternate synthesis chemistries and methods, e.g., the phosphate triester method, which has the advantages of using more stable monomer reagents for synthesis, and of not using tetrazole or its derivatives as condensation catalysts. Heat may be provided by a number of means, including, but not limited to, resistance heaters, visible or infrared light, microwaves, Peltier devices, transfer from fluids or gasses (e.g., via channels or a jacketed system). In some embodiments, heat generated by another component of a synthesis or production facility system (e.g., during a waste neutralization step) is used to provide heat to reaction chambers. In other embodiments, heat is delivered through the use of one or more heated reagents. Delivery of heat to reaction chambers also comprises embodiments wherein heat is created within the reaction chamber, e.g., by magnetic induction or microwave treatment. It is contemplated that heating may be accomplished through a combination of two or more different means.

In some embodiments, the delivery of heat provides substantially uniform heating to two or more reaction chambers. In some embodiments, heating is carried out at a temperature in a range of about 20° C. to about 60° C. The present invention also provides methods for determining an optimum temperature for a particular coupling chemistry. For example, multiple synthesizers are run side-by-side with each machine run at a different temperature. Coupling efficiencies are measured and the optimum temperature for one or more incubations times are determined. In other embodiments, different amounts of heat are delivered to different reaction chambers within a single synthesizer, such that different reaction chemistries or protocols can be run at the same time.

Delivery of heat to a closed system will alter the pressure within the system. It is contemplated that the closed system of the present invention will be configured to tolerate variations in the system pressure (i.e., the pressure within the closed system) related to heating or other energy input to the system. In preferred embodiments, the system (e.g., every component of the system and every junction or seal within the system) will be configured to withstand a range of pressures, e.g., pressures ranging from 0 to at least 1 atm, or about 15 psi. It is contemplated that pressures may be varied between different points within the system. For example, in some embodiments, reagents and waste fluids are moved through the reaction chamber by use of a pressure differential between one end (e.g., an input aperture) and the other (e.g., a drain aperture) of the reaction chamber. In some embodiments, the system of the present invention is configured to use pressure differentials within a pressurized system (e.g., wherein a system segment having lower pressure than another system segment nonetheless has higher pressure than the environment outside the closed system). In some embodiments, the prevention of backward flow of reagents through the system (e.g., in the event of back pressure from a process step such as heating) is controlled by use of pressure. In other embodiments, valves are provided to assist in control of the direction of flow.

In other preferred embodiments, the synthesizer comprises a mixing component configured to mix reaction components, e.g., to facilitate the penetration of reagents into the pores of the solid support. Mixing may be accomplished by a number of means. In some embodiments, mixing is accomplished by forced movement of the fluid through the matrix (e.g., moving it back and forth or circulating it through the matrix using pressure and/or vacuum, or with a fluid oscillator). Mixing may also be accomplished by agitating the contents of the reaction chamber (e.g., stirring, shaking, continuous or pulsed ultra or subsonic waves, See, FIGS. 42A-C and 43A and B). In some preferred embodiments, an agitator is used that avoids the creation of standing waves in the reaction mixture. In some preferred embodiments, the agitator is configured to utilize a reaction vessel surface or reaction support surface (e.g., a surface of a synthesis column) to serve as resonant members to transfer energy into fluid within a reaction mixture. In some embodiments, the matrix is an active component of the mixing system. For example, in some embodiments, the matrix comprises paramagnetic particles that may be moved through the use of magnets to facilitate mixing. In some embodiments, the matrix is an active component of both mixing and heating systems (e.g., paramagnetic particles may be agitated by magnetic control and heated by magnetic induction). It is contemplated that any of these mixing means may be used as the sole means of mixing, or that these mixing components may be used in combination, either simultaneously or in sequence. In preferred embodiments, the heating component and the mixing component are under automated control.

In preferred embodiments, a central control processor is used to automate one or more of the synthesis steps or synthesizer operations. The central control processor may also be configured to interact with one or more other components of a production facility (See below). In some embodiments, the central control processor regulates valves, controlling the timing, volume, a rate of reagent delivery to the reaction chambers. In preferred embodiments, all delivered reagents are controllable for volume within prescribed ranges at each step of the synthesis process within a protocol independent of other steps.

The present invention is not limited by the range of flow rate used for reagent delivery. However, in preferred embodiments, flow rates are 300-500 μL/sec for all reagents.

Table 1, below, provides an example of reagent delivery times (in seconds) and amounts (in microliters) for a single synthesis cycle. Conditions are provided for four different synthesis scales.

TABLE 1

| | 40 nM | 200 nM | 1 μM | 4 μM | Time |
|---|---|---|---|---|---|
| Step | scale | scale | scale | scale | (sec) |
| add acetonitrile | 50 | 150 | 250 | 1000 | 0.5 |
| argon purge | | | | | 1 |
| add deblock | 50 | 150 | 250 | 1000 | 0.5 |
| argon purge | | | | | 1 |
| add deblock | 50 | 150 | 250 | 1000 | 0.5 |
| argon purge | | | | | 1 |
| add deblock | 50 | 150 | 250 | 1000 | 0.5 |
| argon purge | | | | | 1 |
| add deblock | 50 | 150 | 250 | 1000 | 0.5 |
| argon purge | | | | | 1 |
| add acetonitrile | 50 | 150 | 250 | 1000 | 0.5 |
| argon purge | | | | | 1 |
| add amidite and | 15 | 30 | 75 | 300 | 30 × 4 |
| tetrazole | 20 | 45 | 115 | 460 | |
| argon purge | | | | | 1 |
| add cap a | 15 | 30 | 60 | 180 | 1 |
| add cap b | 15 | 30 | 60 | 180 | |
| argon purge | | | | | 1 |
| add oxidizer | 40 | 80 | 180 | 360 | 0.5 |
| argon purge | | | | | 1 |
| add acetonitrile | 100 | 200 | 250 | 1000 | |
| argon purge | | | | | |

In preferred embodiments, with the exception of the amidite coupling step, reaction or wash times are controlled by fluid application rate without additional dwell time prior to purging. This is in contrast to methods used with current commercial synthesizers (e.g., 3900 DNA Synthesizers).

A number of different configurations of the synthesizers of the present invention are provided below with exemplary capacities provided. The present invention is not limited to these specific configurations.

A. Pure Batch, Fully Dedicated Fluidics

Batch size is preferably 96 arrayed reaction chambers in a standard microtiter footprint. Synthesis columns could be either independently filled and inserted into a rack to form the array or, preferably, molded in an arrayed format and filled as a batch. If the latter, then all columns should be of a similar type and synthesis operations are grouped accordingly. Column plates are loaded one at a time and replaced at the end of the synthesis process. In some embodiments, loading and unloading is manual—no transport mechanisms required. In other embodiments, loading and unloading is controlled robotically. Fluid connections from the system to the column tray is either established by the system (moving mechanism) or by the user en mass (fixed dispense). Application of reagents is accomplished by a fixed set of multifunctional reagent dispensers, each incorporating all required reagents: each column has a dedicated multiplexed supply line and no motion devices or fluid connection make/break cycles are required. This approach requires a large number of valves (approximately 1000) and is therefore preferably uses very compact, relatively inexpensive and relatively high reliability valves.

| | |
|---|---|
| Estimated walk away time: | 35 minutes |
| Optimal output per day: | approximately 2496 40-mers |
| Valve count: | 1000 |
| Mechanism level: | none |
| Size: | smallest |

B. Pure Batch: Non-dedicated Fluidics

This system is similar to the pure batch system, but rather than dedicated fluidics for each channel, moving reagent dispense heads are provided. This reduces the valve count but adds mechanism. Also, output per day drops in some scale to the valve reduction. A system with approximately 200 valves would produce about 1056 oligonucleotides/2 shift day. Adding a parallel processing station to achieve 2112/day is an option. Walk away time goes up to approximately 80 minutes.

| | |
|---|---|
| Estimated walkaway time: | 1.3 hours |
| Optimal output per day: | approximately 2112 40-mers |
| Valve count: | 400 |
| Mechanisms level: | moderate |
| Size: | moderate |

C. Modified Batch:

This system is similar in configuration to the non-dedicated fluidics batch system described above, but allows multiple plate positions with the system. Walkaway time improves linearly with the number of plates allowed, throughput and other comments are similar. At increasing levels of resident plates, parallel (400 valve system) with 4 plates resident for each parallel line would allow walk away time of 5 hours. In principle, 4 runs of 8 plates could be completed per day producing 3072 oligonucleotides. A 200-valve system configured similarly could produce 1536.

| | |
|---|---|
| Estimated walkaway time: | 5 hours |
| Optimal output per day: | approximately 1536 40-mers |
| Valve count: | 200 |
| Mechanism level: | moderate |

Size: moderate

D. Continuous Batch:

This system is similar to the above system with the addition of queues for feeding plates and accumulating completed plates. The system requires similar fluid handling but adds plate transport mechanisms. The waste system is more complicated due to plate movement. This system allows direct integration to downstream cleave and deprotect system and allows direct integration to synthesis column packing upstream. Throughput is slightly higher than the modified batch system.

| | |
|---|---|
| Estimated walkaway time: | Limited only by onboard storage |
| Optimal output per day: | approximately 1536 40-mers |
| Valve count: | 200 |
| Mechanism level: | high |
| Size: | large |

E. Continuous Parallel:

Rather than a 96-well format, the columns are prepared and presented in strips of 12 columns. The strips are fed through multiple parallel reagent delivery ports. This approach allows greater spacing between adjacent fluidic elements and allows processing of multiple different column types simultaneously. An additional benefit is the likelihood that a closer approach to the theoretical maximum throughput should be routinely achieved. In this embodiment, throughput per valve would be similar to continuous batch, but tubing of throughput is easier.

| | |
|---|---|
| Estimated walkaway time: | limited only by onboard storage |
| Optimal output per day: | approximately 1536 40-mers |
| Valve count: | 200 |
| Mechanism level: | high |
| Size: | large |

(All valve counts are approximate and assume 2 way valves: with multi-position valves, the counts drop accordingly. Also, some rejection may be possible by ganging operations less critically dependent on precise fluid delivery (washes etc). All throughputs assume a nominal cycle for 1 uM scale. Larger scale(s) would be significantly longer. Smaller scales would be essentially similar. Mixing longer and shorter oligonucleotides will drive throughputs to that presented by the longer oligonucleotides).

The synthesizers of the present invention also provide components to reduce or eliminate undesired emissions. A problem with currently available synthesizers is the emission of undesirable gaseous or liquid materials that pose health, environmental, and explosive hazards. Such emissions result from both the normal operation of the instrument and from instrument failures. Emissions that result from instrument failures cause a reduction or loss of synthesis efficiency and can provoke further failures and/or complete synthesizer failure. Correction of failures may require taking the synthesizer off-line for cleaning and repair. The present invention provides nucleic acid synthesizers with components that reduce or eliminate unwanted emissions and that compensate for and facilitate the removal of unwanted emissions, to the extent that they occur at all. The present invention also provides waste handling systems to eliminate or reduce exposure of emissions to the users or the environment. Such systems find use with individual synthesizers, as well as in large-scale synthesis facilities comprising many synthesizers (e.g. arrays of synthesizers).

Whether a system used is open or closed, oligonucleotide synthesis involves the use of an array of hazardous materials, including but not limited to methylene chloride, pyridine, acetic anhydride, 2,6-lutidine, acetonitrile, tetrahydrofurane, and toluene. These reagents can have a variety of harmful effects on those who may be exposed to them. They can be mildly or extremely irritating or toxic upon short-term exposure; several are more severely toxic and/or carcinogenic with long-term exposure. Many can create a fire or explosion hazard if not properly contained. In addition, many of these chemicals must be assessed for emissions from normal operations, e.g for determining compliance with OSHA or environmental agency standards. Malfunction of a system, e.g., as recited above, increases such emissions, thereby increasing the risk of operator exposure, and increasing the risk that an instrument may need to be shut down until risk to an operator is reduced and until any regulatory requirements for operation are met.

Emission or leakage of reagents during operation can have consequences beyond risks to personnel and to the environment. As noted above, instruments may need to be removed from operation for cleaning, leading to a temporary decrease in production capacity of a synthesis facility. Further, any emission or leakage may cause damage to parts of the instrument or to other instruments or aspects of the facility, necessitating repair or replacement of any such parts or aspects, increasing the time and cost of bringing an instrument back into operation. Failure to address emissions or leakage concerns may lead to additional expenses for operation of a facility, e.g., costs for increased or improved fire or explosion containment measures, and addition of costs associated with the elimination of any instrument systems or wiring that have not been determined to be safe for use in such hazardous locations (e.g., by reference to controlling codes, such as electrical codes, or codes covering operations in the presence of flammable and combustible liquids).

The synthesizers of the present invention provide a number of novel features that dramatically improve synthesizer performance and safety compared to available synthesizers. These novel features work both independently and in conjunction to provide enhanced performance. For example, the present invention reduces exposure by improving collection and disposal of emissions that occur during the normal operation of various synthesis instruments. In another embodiment, the present invention reduces exposure by improving aspects of the instrument to reduce risk of malfunctions leading to reagent escape from the system, e.g., through leakage, overflow or other spillage.

For example, in some embodiments, the present invention provides a means of collecting emissions from the interior of synthesizers by providing a reagent dispensing station. In one embodiment, the reagent dispensing station is an integral part of the base 2 of the synthesizer, as illustrated in FIGS. 47A and 47B. In some embodiments, the reagent dispensing station provides an enclosure for collecting emitted gasses. In some embodiments, the enclosure is created by the provision of a panel 73 to enclose a portion of base 2 containing reagent reservoirs 72, as illustrated in FIG. 47B. In some embodiments, the panel 73 is movable for easy access to reagent reservoirs. In some embodiments, it is removeably attached. Removable attachment may be accomplished by any suitable means, such as through the use of VELCRO, screws, bolts, pins, magnets, temporary adhesives, and the like. In preferred embodiments, at least a portion of the panel 18 is slidably moveable. In preferred embodiments, at least a portion of panel 18 is transparent. In some embodiments, the enclosure of the reagent dispensing station comprises a viewing window that is not in a panel 73.

In some embodiments, the enclosure comprises ventilation tubing. In preferred embodiments, panel 73 comprises a ventilation port 74, e.g., for attachment to ventilation tubing. Since reagent vapors are typically heavier than air, in preferred embodiments, the ventilation tubing is attached at the bottom for the enclosure. In a particularly preferred embodiment, the ventilation port is positioned toward the rear of the instrument.

In some embodiments, the enclosure further comprises an air inlet. In a preferred embodiment, a clearance 75 between the panel 73 and the base 2 provides an air inlet. In a particularly preferred embodiment, the air inlet is positioned toward the front of the instrument.

The location of the ventilation port 74 and air inlet is not limited to the panel 73. For example, in an alternative embodiment, the reagent dispensing station comprises a stand for holding the reagent bottles and ventilation tubing, wherein the stand holds the reagent reservoirs and the ventilation tubing removes emitted gases.

Ventilation may be continuous or under the control of an operator. For example, in some embodiments, when the panel 73 is in a closed position, ventilation occurs continuously through the ventilation port 74 or at regular intervals. In other embodiments, an operator may manually activate ventilation prior to opening the panel 73. In still other embodiments, ventilation occurs in an automated fashion immediately prior to the opening of panel 73. For example, where the opening of panel 73 is controlled by a computer processor, activation of the “open” routine triggers ventilation prior to the physical opening of panel 73. In still other embodiments, the contents of the reagent containers are monitored by a sensor and the ventilation is triggered when one or more of the reagent containers are depleted. In some embodiments, the panel 73 is also automatically open, indicating the need for additional reagents and/or allowing an automated reagent container delivery system to supply reagents to the system.

In some embodiments, multiwell plates (e.g. 96 well, 384 well, 1536 well, etc) are employed with the synthesizers of the present invention. In certain embodiments, the synthesizers are parts of a full automated process such that oligonucleotides are produced without human interaction. In some embodiments, the oligonucleotides move through the synthesis component, and processing components, on rails.

2. Automated and Fail-Safe Reagent Supply

In some embodiments, the DNA synthesizers in the oligonucleotide synthesis component further comprise an automated reagent supply system. The automated reagent supply system delivers reagents necessary for synthesis to the synthesizers from a central supply area. In some embodiments, the central supply area is provided in an isolated room equipped for accommodating leakage, fires, and explosions without threatening other portions of the synthesis facility, the environment, or humans. Where the central supply area provides reagents for multiple synthesizers, in some embodiments, the system is configured to allow banks of synthesizer or individual synthesizer to be removed from the system (e.g., for maintenance or repair) without interrupting activity at other synthesizers. Thus, the present invention provides an efficient fail-safe reagent delivery system.

For example, in some embodiments, acetonitrile is supplied via tubing (e.g., stainless steel or TEFLON tubing) through the automated supply system. De-blocking solution may also be supplied directly to DNA synthesizers through tubing. In some preferred embodiments, the reagent supply system tubing is designed to connect directly to the DNA synthesizers without modifying the synthesizers. Additionally, in some embodiments, the central reagent supply is designed to deliver reagents at a constant and controlled pressure. The amount of reagent circulating in the central supply loop is maintained at 8 to 12 times the level needed for synthesis in order to allow standardized pressure at each instrument. The excess reagent also allows new reagent to be added to the system without shutting down. In addition, the excess of reagent allows different types of pressurized reagent containers to be attached to one system. The excess of reagents in one centralized system further allows for one central system for chemical spills and fire suppression.

In some embodiments, the DNA synthesis component includes a centralized argon delivery system. The system includes high-pressure argon tanks adjacent to each bank of synthesizers. These tanks are connected to large, main argon tanks for backup. In some embodiments, the main tanks are run in series. In other embodiments, the main tanks are set up in banks. In some embodiments, the system further includes an automated tank switching system. In some preferred embodiments, the argon delivery system further comprises a tertiary backup system to provide argon in the case of failure of the primary and backup systems.

In some embodiments, one or more branched delivery components are used between the reagent tanks and the individual synthesizers or banks of synthesizers. For example, in some embodiments, acetonitrile is delivered through a branched metal structure (e.g., the structure described in FIG. 56). Where more than one branched delivery component is used, in preferred embodiments, each branched delivery component is individually pressurized.

The present invention is not limited by the number of branches in the branched delivery component. In preferred embodiments, each branched delivery component (100) contains ten or more branches (101). Reagent tanks may be connected to the branched delivery components using any number of configurations. For example, in some embodiments, a single reagent tank is matched with a single branched component. In other embodiments, a plurality of reagent tanks is used to supply reagents to one or more branched components. In some such embodiments, the plurality of tanks may be attached to the branched components through a single-feed line, wherein one or a subset of the tanks feeds the branched components until empty (or substantially empty), whereby a second tank or subset of tanks is accessed to maintain a continuous supply of reagent to the one or more branched components. To automate the monitoring and switching of tanks, an ultrasonic level sensor may be applied.

In some embodiments, each branch of the branched delivery component provides reagent to one synthesizer or to a bank of synthesizers through connecting tubing (102). In preferred embodiments, tubing is continuous (i.e., provides a direct connection between the delivery branch and the synthesizer). In some preferred embodiments, the tubing comprises an interior diameter of 0.25 inches or less (e.g., 0.125 inches). In some embodiments, each branch contains one or more valves (preferably one). While the valve may be located at any position along the delivery line, in preferred embodiments, the valve is located in close proximity to the synthesizer. In other embodiments, reagent is provided directly to synthesizers without any joints or valves between the branched delivery component and the synthesizers.

In some embodiments, the solvent is contained in a cabinet designed for the safe storage of flammable chemicals (a “flammables cabinet”) and the branched structure is located outside of the cabinet and is fed by the solvent container through tubing passed through the wall of the cabinet. In other embodiments, the reagent and branched system is stored in an explosion proof room or chamber and the solvent is pumped via tubing through the wall of the explosion proof room. In preferred embodiments, all of the tubing from each of the branches is fed through the wall in at a single location (e.g., through a single hole (103) in the wall (104)).

The reagent delivery system of the present invention provides several advantages. For example, such a system allows each synthesizer to be turned off (e.g., for servicing) independent of the other synthesizers. Use of continuous tubing reduces the number of joints and couplings, the areas most vulnerable to failure, between the reagent sources and the synthesizers, thereby reducing the potential for leakage or blockage in the system. Use of continuous tubing through inaccessible or difficult-to-access areas reduces the likelihood that repairs or service will be needed in such areas. In addition, fewer valves results in cost savings.

In some embodiments, the branched tubing structure further provides a sight glass (105). In preferred embodiments, the sight glass is located at the top of the branched delivery structure. The sight glass provides the opportunity for visual and physical sampling of the reagent. For example, in some embodiments, the sight glass includes a sampling valve (106) (e.g., to collect samples for quality control). In some embodiments, the site glass serves as a trap for gas bubbles, to prevent bubbles from entering the connecting tubing (102). In other embodiments, the sight glass contains a vent (e.g., a solenoid valve) for de-gassing of the system (107). In some embodiments, scanning of the sight glass (e.g., spectrophotometrically) and sampling are automated. The automated system provides quality control and feedback (e.g.. the presence of contamination).

In other embodiments, the present invention provides a portable reagent delivery system. In some embodiments, the portable reagent delivery system comprises a branched structure connected to solvent tanks that are contained in a flammables cabinet. In preferred embodiments, one reagent delivery system is able to provide sufficient reagent for 40 or more synthesizers. These portable reagent delivery systems of the present invention facilitate the operation of mobile (portable) synthesis facilities. In another embodiment, these portable reagent delivery systems facilitate the operation of flexible synthesis facilities that can be easily re-configured to meet particular needs of individual synthesis projects or contracts. In some embodiments, a synthesis facility comprises multiple portable reagent delivery systems.

3. Waste Collection

In some embodiments, the DNA synthesis component further comprises a centralized waste collection system. The centralized waste collection system comprises cache pots for central waste collection. In some embodiments, the cache pots include level detectors such that when waste level reaches a preset value, a pump is activated to drain the cache into a central collection reservoir. In preferred embodiments, ductwork is provided to gather fumes from cache pots. The fumes are then vented safely through the roof, avoiding exposure of personnel to harmful fumes. In preferred embodiments, the air handling system provides an adequate amount of air exchange per person to ensure that personnel are not exposed to harmful fumes. The coordinated reagent delivery and waste removal systems increase the safety and health of workers, as well as improving cost savings.

In some embodiments, the solvent waste disposal system comprises a waste transfer system. In some preferred embodiments, the system contains no electronic components. In some preferred embodiments, the system comprises no moving parts. For example, in some embodiments, waste is first collected in a liquid transfer drum (200) designed for the safe storage of flammable waste (See FIG. 57 for an exemplary waste disposal system). In some embodiments, waste is manually poured into the drum through a waste channel (201). In preferred embodiments, solvent waste is automatically transported (e.g., through tubing) directly from synthesizers to the drum (200). To drain the liquid transfer drum (200), argon is pumped from a pressurized gas line (202) into the drum through a first opening (203), forcing solvent waste out an output channel (204) at a second opening (205) (e.g., through tubing) into a centralized waste collection area. In preferred embodiments, the argon is pumped at low pressure (e.g., 3-10 pounds per square inch (psi), preferably 5 psi or less). In some embodiments, the drum (200) contains a sight glass (207) to visualize the solvent level. In some embodiments, the level is visualized manually and the disposal system is activated when the drum (200) has reached a selected threshold level (207). In other embodiments, the level is automatically detected and the disposal system is automatically activated when the drum (200) has reached the threshold level (207).

The solvent waste transfer system of the present invention provides several advantages over manual collection and complex systems. The solvent waste system of the present invention is intrinsically safe, as it can be designed with no moving or electrical parts. For example, the system described above is suitable for use in Division I/Class I space under EPA regulations.

Some process steps may put out caustic waste. For example, deprotection of synthesized oligonucleotides generally includes treatment with NH4OH. In some embodiments, caustic waste is neutralized before disposal, e.g., to a sanitary sewer. In preferred embodiments, the neutralization of the waste is checked (e.g.; by measurement of pH) to ensure that it is in an appropriate condition for disposal via the intended system (e.g., the sanitary sewer system).

In some embodiments, waste from each deprotection station is neutralized before collection to a centralized waste collection or disposal system. In other embodiments, caustic waste from a plurality of deprotection stations is collected before neutralization.

By way of example, and not intended as a limitation, the following provides a description for one embodiment of a centralized collection and neutralization system for caustic waste. The system may comprise collection of caustic waste from one or more stations in a tank, e.g., a carboy. In some embodiments, the amount of neutralizing reagent required to neutralize a defined amount of caustic waste is calculated, based on the volume and content of the waste. In some embodiments, the calculated amount of neutralizing reagent is added after collection of the waste. In preferred embodiments, the calculated amount of neutralizing reagent is provided in the carboy, such that when the carboy is full or when the combined volume of the neutralizer and waste reaches a predetermined volume, the waste has been neutralized.

In one embodiment, the carboy is provided with a pH probe for measurement of the pH of the collected waste. In some embodiments, the system provides a means of altering the pH of the collected waste. In preferred embodiments, the altering of the pH occurs in response to a measured pH value for the collected waste. For example, if the pH is determined to be outside a certain range, (e.g., if it does not fall between, for example, pH 7 and pH 9), the system provides a reagent selected to adjust the pH to the selected range (e.g., if the pH is found to be high, the system dispenses an acidic solution for neutralization; if the pH is low, the system dispenses, a basic solution for neutralization). When the pH comes into the selected range, the system shuts off the dispenser. For the step of dispensing a neutralizing reagent, any system suitable for the controlled delivery of a reagent is contemplated. For example, discharge may be accomplished via a mechanical dispenser, or discharge can be accomplished via non-mechanical means, e.g., via control of air pressure.

In some embodiments, neutralization treatment is provided to the collected waste in bulk, e.g., when the carboy is full or when it reaches a predetermined threshold level. In other embodiments, neutralization is periodic. In some embodiments, periodic neutralization is set to occur at particular times, e.g., at particular times of day, or whenever a particular interval of time has passed since the last treatment. In other embodiments, periodic treatment is set to respond to a condition of the waste container, such as whenever a new addition of waste material occurs, or whenever the pH is not within the selected range. In yet other embodiments, periodic treatment occurs based on a combination of these or other factors.

In a preferred embodiment, the carboy is provided with a means for mixing, such as a stirrer or agitator. In some embodiments, the system comprises a device for keeping a precipitate suspended. In some embodiments, the system provides a filter for removing precipitates, particulates or other non-liquid matter in the collected waste. In other preferred embodiments, the system provides a means of venting gasses. In particularly preferred embodiments, the gasses are collected for disposal through a centralized ventilation system.

4. Centralized Control System

In some embodiments, all of the DNA synthesizers in the synthesis component are attached to a centralized control system. The centralized control system controls all areas of operation, including, but not limited to, power, pressure, reagent delivery, waste, and synthesis. In preferred embodiments, the centralized control system is operably linked to data (enterprise) management system (See, below). In other preferred embodiments, the centralized control system (for oligonucleotide synthesis) is operably linked to the centralized control network (for oligonucleotide processing. The combination of the centralized control system and centralized control network is referred to as the shop floor control system. In some preferred embodiments, the centralized control system includes a clean electrical grid with uninterrupted power supply. Such a system minimizes power level fluctuations. In additional preferred embodiments, the centralized control system includes alarms for air flow, status of reagents, and status of waste containers. The alarm system can be monitored from the central control panel. The centralized control system allows additions, deletions, or shutdowns of one synthesizer or one block of synthesizers without disrupting operations of other instruments. The centralized power control allows user to turn instruments off instrument by instrument, bank by bank, or the entire module. In some embodiments, the centralized control system comprises enterprise software (e.g. Oracle, PeopleSoft, etc.).

B. Oligonucleotide Processing Components

In some embodiments, the automated DNA production process further comprises one or more oligonucleotide production components, including, but not limited to, an oligonucleotide cleavage and deprotection component, an oligonucleotide purification component, a dry-down component, a desalting component, a dilution and fill component, and a quality control component. In preferred embodiments, the synthesis component is integrated with the oligonucleotide processing components, and other components such as the order entry component discussed above (see also FIG. 58b). Preferably, the components are operably linked for data sharing, product tracking and control. It is also preferred that the various components are operably linked such that oligonucleotides are processed with limited human interaction. A general overview of how the components are operably connected, in some embodiments, is provided in FIG. 58a. Particular embodiments for process and data flow within and between the various processing components are shown in FIGS. 58b-58k.

Preferably the oligonucleotide components are automated, at least in part, in order to improve efficiencies and reduce human errors. In preferred embodiments, 96 well (or 384 well) plates are used through out the entire system (e.g. from initial synthesis to dilute and fill), such that individual columns do not have to be transferred between different sized plates. In other embodiments, samples are maintained in a closed-circuit tubing for synthesis and one or more additional components (e.g., cleavage and deprotection, purification, etc.) such that a solution carrying the sample passes through a plurality of reaction zones where the tubing is heated, agitated, accessed by other tubing to deliver necessary reagents, etc. without ever being removed from the tubing or exposed to the ambient environment. Such systems facilitate high-throughput production if detection assays.

1. Oligonucleotide Cleavage and Deprotection

After synthesis is complete, the oligonucleotides synthesis columns are moved to the cleavage and deprotection station. In some embodiments, the transfer of oligonucleotides to this station is automated and controlled by robotic automation. In some embodiments, the entire cleavage and deprotection process is performed by robotic automation. In some embodiments, NH₄OH for deprotection is supplied through the automated reagent supply system.

Accordingly, in some embodiments, oligonucleotide deprotection is performed in multi-sample containers (e.g., 96 well covered dishes) in an oven. This method is designed for the high-throughput system of the present invention and is capable of the simultaneous processing of large numbers of samples. This method provides several advantages over the standard method of deprotection in vials. For example, sample handling is reduced (e.g., labeling of vials dispensing of concentrated NH₄OH to individual vials, as well as the associated capping and uncapping of the vials, is eliminated). This reduces the risks of contamination or mislabeling and decreases processing time. Where such methods are used to replace human pipetting of samples and capping of vials, the methods save many labor hours per day. The method also reduces consumable requirements by eliminating the need for vials and pipette tips, reduces equipment needs by eliminating the need for pipettes, and improves worker safety conditions by reducing worker exposure to ammonium hydroxide. The potential for repetitive motion disorders is also reduced. Deprotection in a multi-well plate further has the advantage that the plate can be directly placed on an automated desalting apparatus (e.g., TECAN Robot).

During the development of the present invention, the plate was optimized to be functional and compatible with the deprotection methods. In some embodiments, the plate is designed to be able to hold as much as two milliliters of oligonucleotide and ammonium hydroxide. If deep well plates are used, automated downstream processing steps may need to be altered to ensure that the full volume of sample is extracted from the wells. In some embodiments, the multi-well plates used in the methods of the present invention comprise a tight sealing lid/cover to protect from evaporation, provide for even heating, and are able to withstand temperatures and pressures necessary for deprotection. Attempts with initial plates were not successful, having problems with lids that were not suitably sealed and plates that did not withstand deprotection temperatures.

In some embodiments (e.g., processing of target and INVADER oligonucleotides), oligonucleotides are cleaved from the synthesis support in the multi-well plates. In other embodiments (e.g., processing of probe oligonucleotides), oligonucleotides are first cleaved from the synthesis column and then transferred to the plate for deprotection.

In preferred embodiments, the present invention provides devices and systems for automated and semi-automated cleavage and/or protections. Preferably, the cleave and deprotect device is configured to hold 96 synthesis columns (e.g. in an 8 by 12 plate). It is also preferred that reagents, such as ammonium hydroxide, may be contacted with the synthesis columns (or other columns containing oligonucleotides) with minimal or no exposure of the reagents to the ambient environment. Also, the cleave and deprotect device is preferably configured to allow the automatic dispersement of reagents into the synthesis columns at periodic intervals in order to facilitate cleavage. For example, the present invention provides a system comprising a series of fluid dispensers (e.g. a series of fluid dispensers), a software application (e.g. Unicorn software) that instructs the fluid dispenser (e.g. to engage the synthesis columns once the rack holding the columns is inserted into the automated device), and a cleave and deprotect device for holding the synthesis columns. In other preferred embodiments, the cleave and deprotect device allows reagents such as ammonium hydroxide to pass through the synthesis column and info a receive plate below (e.g. a 96 well receive plate that collects the reagents and oligonucleotdies as they are cleaved from the synthesis columns). The receiving plate may be in a 96 well, 384 well, or any other type of format. In other preferred embodiments, the fluid is dispensed in lines that end with fluid column connections (e.g. FIG. 60A, number 106), or the fluid column connections are part of the cleave and deprotect device.

FIG. 60 shows exemplary components of an automated cleave and deprotect system. FIGS. 60A and 60B show a side view of a cleave and deprotect device. FIG. 60A shows the fluid column connections in the down position (e.g. engaged with the synthesis columns), and FIG. 60B shows the fluid column connections in the up position. A brief description of various part of the cleavage and/or deprotect device as shown in FIGS. 60A-H is provided. The catch plate 100 is preferably a deep well plate. This catch plate collects the oligonucleotides as they come off the column due to exposure to ammonium hydroxide. The catch plate may, for example, be a 96 well plate. This plate can them be moved to a further processing step (e.g. a deprotection step, where the plate is covered and then heat is applied). Columns 102 (e.g. synthesis columns) are held in column holder 104 (See FIG. 60A). A top view of one particular column holder is provided in FIG. 60E. Fluid column connection 106 allows liquid to be dispensed to the columns with minimal or no exposure of reagents to the ambient environment. Fluid column connections may be made from any suitable material, and have various parts that facilitate connection with the columns (see FIG. 60F). Connection 106 has a plurality of rings 108 (2 shown in FIG. 60A). Either one or both rings engage the interior surface 10 of column. The rings 108 are radiused so that they form a releasable seal whey they engage surface 110. It is appreciated that when rings 108 are radiused a releasable seal is formed even if columns 108 are at an angle other than a 9 degree angle to column holder 104. Even if there is a small amount of misalignment between the column 102 and connection 106 there is a substantially airtight and water tight seal formed.

Columns 102 when releasably sealed to connections 106 move horizontally and/or vertically as a block in some embodiments. When the columns 102 rise up with connections they contact stripper plate 112 which has an aperature 114 which permits connection 106 to pass therethrough, but acts as a limit stop when lip 118 contacts stripper block plate surface 120 (see Stripper plate in FIG. 60A and FIG. 60C). Aperature 114 is large enough to let the connection 106 to ride through it but is smaller than the diameter of lip 118. Actuation of connection holder 122 for movement along the guide shafts 124 (see FIGS. 60A and 60H) which are secured to base 126. The base of the machine is shown in FIGS. 60A and 60G. Finally the dispense tip holder is shown in FIGS. 60A and 60D.

In some embodiments, software, such as Unicorn Software, controls the amount and timing of reagents dispensed into the synthesis columns. For example, a 45 minute program may be run that periodically dispenses ammonium hydroxide into the synthesis columns at timed intervals in order to cleave the oligonucleotides off of the synthesis columns. In certain embodiments, the automated cleavage and deprotection system is configured to work with a polyplex machine (e.g. software allows an interface between the cleavage and deprotection).

In certain embodiments, fast deprotection chemistry is utilized to increase the rate at which oligonucleotide are manufactured. For example, oligonucleotdies may be synthesized with Proligo Tac Amidites that have a tert.-butylphenoxy-acetyl “tac” base protecting group. This protecting group decreases cleavage and deprotection time of the final oligo from about eight hours to about 15 minutes at 55° C., or two hours at room temperature when compared with standard base protecting groups. Rapid deprotection results in less exposure to ammonia and reduced risk of hydrolysis. Also, this type of fast deprotection chemistry may be used with the autocleave device of the present invention. For example, the autocleave device may be heated up to the deprotecting temperature (e.g. 60 degrees Celsius), and both cleavage and deprotection can occur in the same column in the autocleave device. This allows, for example, the cleaved and deprotected to go straight into a purification column (e.g. C₁₈ column).

2. Oligonucleotide Purification

In some embodiments, following deprotection and cleavage from the solid support, oligonucleotides are further purified. In certain embodiments, the purification step is not necessary (e.g. the synthesis and cleave and deprotect steps yield a sufficiently pure oligonucleotide preparation, or the detection assay being produced does not require an oligonucleotide purification step). Any suitable purification method may be employed when purification is desired, including, but not limited to, high pressure liquid chromatography (HPLC) (e.g., using reverse phase C18 and ion exchange), reverse phase cartridge purification, probe capture, and gel electrophoresis. However, in preferred embodiments, purification is carried out using ion exchange HPLC chromatography.

In some embodiments, multiple HPLC instruments are utilized, and integrated into banks (e.g., banks of 8 HPLC instruments). Each bank is referred to as an HPLC module. Each HPLC module consists of an automated injector (e.g., including, but not limited to, Leap Technologies 8-port injector) connected to each bank of automated HPLC instruments (e.g., including, but not limited to, Beckman-Coulter HPLC instruments). The automatic Leap injector can handle four 96-well plates of cleaved and deprotected oligonucleotides at a time. The Leap injector automatically loads a sample onto each of the HPLCs in a given bank. The use of one injector with each bank of HPLC provides the advantage of reducing labor and allowing integrated processing of information. In preferred embodiments, reagents are supplied directly to the HPLC instruments via a solvent delivery component (See, e.g. FIG. 56).

In some embodiments, oligonucleotides are purified on an ion exchange column using a salt gradient. Any suitable ion exchange functionality or support may be utilized, including but not limited to, Source 15 Q ion exchange resin (Pharmacia). Any suitable salt may be utilized for elution of oligonucleotides from the ion exchange column, including but not limited to, sodium chloride, acetonitrile, and sodium perchlorate. However, in preferred embodiments, a gradient of sodium perchlorate in acetonitrile and sodium acetate is utilized.

In some embodiments, the gradient is run for a sufficient time course to capture a broad range of sizes of oligonucleotides. For example, in some embodiments, the gradient is a 54 minute gradient carried out using the method described in Tables 3 and 4. Table 3 describes the HPLC protocol for the gradient. The time column represents the time of the operation. The module column represents the equipment that controls the operation. The function column represents the function that the HPLC is performing. The value column represents the value of the HPLC function at the time specified in the time column. Table 4 describes the gradient used in HPLC purification. The column temperature is approximately 65° C. Buffer A is 20 mM Sodium Perchlorate, 20 mM Sodium Acetate, 10 percent Acetonitrile, pH 7.35. Buffer B is 600 mM Sodium Perchlorate, 20 mM Sodium Acetate, 10 percent Acetonitrile, pH 7-8.

In some embodiments, the gradient is shortened. In preferred embodiments, the gradient is shortened so that a particular gradient range suitable for the elution of a particular oligonucleotide being purified is accomplished in a reduced amount of time. In other preferred embodiments, the gradient is shortened so that a particular gradient range suitable for the elution of any oligonucleotide having a size within a selected size range is accomplished in a reduced amount of time. This latter embodiment provides the advantages that the worker performing HPLC need not have foreknowledge of the size of an oligonucleotide within the selected size range, and the protocol need not be altered for purification of any oligonucleotide having a size within the range.

In a particularly preferred embodiment, the gradient is a 34 minute gradient described in the Tables 4 and 5. The parameters and buffer compositions are as described for Tables 3 and 4 above. Reducing the gradient to 34 minutes increases the capacity of synthesis per HPLC instrument and reduces buffer usage by 50% compared to the 54 minute protocol described above. The 34 minute HPLC method of the present invention has the further advantage of being optimized to be able to separate oligonucleotides of a length range of 23-39 nucleotides without any changes in the protocol for the different lengths within the range. Previous methods required changes for every 2-3 nucleotide change in length.. In yet other embodiments, the gradient time is reduced even further (e.g., to less than 30 minutes, preferably to less than 20 minutes, and even more preferably, to less than 15 minutes). Any suitable method may be utilized that meets the requirements of the present invention (e.g., able to purify a wide range of oligonucleotide lengths using the same protocol).

In some embodiments, separate sets of HPLC conditions, each selected to purify oligonucleotides within a different size range, may be provided (e.g., may be run on separate HPLCs or banks of HPLCs). Thus, in some embodiments of the present invention, a first bank of HPLCs are configured to purify oligonucleotides using a first set of purification conditions (e.g., for 23-39 mers), while second and third banks are used for the shorter and longer oligonucleotides. Use of this system allows for automated purification without the need to change any parameters from purification to purification and decreases the time required for oligonucleotide production.

In some embodiments, the HPLC station is equipped with a central reagent supply system. In some embodiments, the central reagent system includes an automated buffer preparation system. The automated buffer preparation system includes large vat carboys that receive pre-measured reagents and water for centralized buffer preparation. The buffers (e.g., a high salt buffer and a low salt buffer) are piped through a circulation loop directly from the central preparation area to the HPLCs. In some embodiments, the conductivity of the solution in the circulation loop is monitored to verify correct content and adequate mixing. In addition, in some embodiments, circulation lines are fitted with venturis for static mixing of the solutions as they are circulated through the piping loop. In still further embodiments, the circulation lines are fitted with 0.05 μm filters for sterilization.

In some preferred embodiments, the HPLC purification step is carried out in a clean room environment. The clean room includes a HEPA filtration system. All personnel in the clean room are outfitted with protective gloves, hair coverings, and foot coverings.

In preferred embodiments, the automated buffer prep system is located in a non-clean room environment and the prepared buffer is piped through the wall into the clean room.

Each purified oligonucleotide is collected into a tube (e.g., a 50-ml conical tube) in a carrying case in the fraction collector. Collection is based on a set method, which is triggered by an absorbance rate change, level, or threshold within a predetermined time window. In some embodiments, the method uses a flow rate of 5 ml/min (the maximum rate of the pumps is 10 ml/min.) and each column is automatically washed before the injector loads the next sample.

(Det=detector; % B=percent of buffer B; flow rate values in ml/min)

TABLE 3
54 Minute HPLC Method

| Time (min) | Module | Function | Value | Duration (min) |
|---|---|---|---|---|
| 0 | Pump | % B | 22.00 | 4.0 |
| 0 | Det 166-3 | Autozero ON | | |
| 0 | Det 166-3 | Relay ON | 3.0 | 0.10 |
| 4 | Pump | % B | 37.00 | 43.00 |
| 47 | Pump | % B | 100.00 | 0.50 |
| 47.5 | Pump | Flow Rate | 7.5 | 0.00 |
| 50.0 | Pump | % B | 5.0 | 0.50 |
| 53.45 | Det 166-3 | Stop Data | | |

TABLE 4
54 Minute HPLC Method

| Time | | | Gradient | | Flow Rate |
|---|---|---|---|---|---|
| | 0 | | 5% B/95% A | | 5 ml/min |
| | 0-4 | min | 5-22% | B | 5 ml/min |
| | 4-47 | min | 22-37% | B | 5 ml/min |
| | 47-47.5 | min | 37-100% | B | 7.5 ml/min |
| | 47.5-50 | min | 100% | B | 7.5 ml/min |
| | 50-50.5 | min | 100-5% | B | 7.5 ml/min |
| | 50.5-53.5 | min | 5% | B | 7.5 ml/min |

TABLE 5
34 Minute HPLC Method

| Time (min) | Module | Function | Value | Duration |
|---|---|---|---|---|
| 0 | Pump | % B | 26.00 | 2.0 |
| 0 | Det 166-3 | Autozero ON | | |
| 0 | Det 166-3 | Relay ON | 3.0 | 0.10 |
| 2 | Pump | % B | 36.00 | 27.00 |
| 29 | Pump | % B | 100.00 | 0.50 |
| 29.5 | Pump | Flow Rate | 7.5 | 0.00 |
| 32 | Pump | % B | 5.0 | 0.50 |
| 33.45 | Det 166-3 | Stop Data | | |

TABLE 6
34 Minute HPLC Method

| Time | | Gradient | | Flow Rate |
|---|---|---|---|---|
| 0 | | 5% B/95% A | | 5 ml/min |
| 0-2 | min | 5-26% | B | 5 ml/min |
| 2-29 | min | 26-36% | B | 5 ml/min |
| 29-29.5 | min | 36-100% | B | 6.5 ml/min |
| 29.5-32 | min | 100% | B | 7.5 ml/min |
| 32-32.5 | min | 100-5% | B | 7.5 ml/min |
| 32.5-33.5 | min | 5% | B | 7.5 ml/min |

3. Dry-Down Component

When the fraction collector is full of eluted oligonucleotides, they are transferred (e.g., by automated robotics or by hand) to a drying station. For example, in some embodiments, the samples are transferred to customized racks for Genevac centrifugal evaporator to be dried down. In preferred embodiments, the Genevac evaporator is equipped with racks designed to be used in both the Genevac and the subsequent desalting step. The Genevac evaporator decreases drying time, relative to other commercially available evaporators, by 60%.

4. Desalting Component

In some embodiments, following HPLC, oligonucleotides are desalted. In other embodiments, oligonucleotides are not HPLC purified, but instead proceed directly from deprotection to desalting. In some embodiments, the desalting stations have TECAN robot systems for automated desalting. The system employs a rack that has been designed to fit the TECAN robot and the Genevac centrifugal evaporator without transfer to a different rack or holder. The racks are designed to hold the different sizes of desalting columns, such as the NAP-5 and NAP-10 columns. The TECAN robot loads each oligonucleotide onto an individual NAP-5 or NAP-10 column, supplies the buffer, and collects the eluate. If desired, desalted oligonucleotides may be frozen or dried down at this point.

In some embodiments, following desalting, INVADER and target oligonucleotides are analyzed by mass spectroscopy. For example, in some embodiments, a small sample from the desalted oligonucleotide sample is removed (e.g., by a TECAN robot) and spotted on an analysis plate, which is then placed into a mass spectrometer. The results are analyzed and processed by a software routine. Following the analysis, failed oligonucleotides are automatically reordered, while oligonucleotides that pass the analysis are transported to the next processing step. This preliminary quality control analysis removes failed oligonucleotides earlier in the processing, thus resulting in cost savings and improving cycle times.

5. Oligonucleotide Dilution and Fill Component

In some embodiments, the oligonucleotide production process further includes a dilute and fill module. In some embodiments, each module consists of three automated oligonucleotide dilution and normalization stations. Each station consists of a network-linked computer and an automated robotic system (e.g., including but not limited to Biomek 2000). In one embodiment, the pipetting station is physically integrated with a spectrophotometer to allow machine handling of every step in the process. All manipulations are carried out in a HEPA-filtered environment. Dissolved oligonucleotides are loaded onto the Biomek 2000 deck the sequence files are transferred into the Biomek 2000. The Biomek 2000 automatically transfers a sample of each oligonucleotide to an optical plate, which the spectrophotometer reads to measure the A260 absorbance. Once the A260 has been determined, an Excel program integrated with the Biomek software uses absorbance and the sequence information to prepare a dilution table for each oligonucleotide. The Biomek employs that dilution table to dilute each oligonucleotide appropriately. The instrument then dispenses oligonucleotides into an appropriate vessel (e.g., 1.5 ml microtubes).

In some preferred embodiments, the automated dilution and fill system is able to dilute different components of a kit (e.g., INVADER and probe oligonucleotides) to different concentrations. In other preferred embodiments, the automated dilution and fill module is able to dilute different components to different concentrations specified by the end user.

6. Quality Control Component

In some embodiments, oligonucleotides undergo a quality control assay before distribution to the user. The specific quality control assay chosen depends on the final use of the oligonucleotides. For example, if the oligonucleotides are to be used in an INVADER SNP detection assay, they are tested in the assay before distribution.

In some embodiments, each SNP set is tested in a quality control assay utilizing the Beckman Coulter SAGIAN CORE System. In some embodiments, the results are read on a real-time instrument (e.g., a ABI 7700 fluorescence reader). The QC assay uses two no target blanks as negative controls and five untyped genomic samples as targets. For consistency, every SNP set is tested with the same genomic samples. In preferred embodiment, the ADS system is responsible for tracking tubes through the QC module. Thus, in some embodiments, if a tube is missing, the ADS program discards, reorders, or searches for the missing tube.

In some preferred embodiments, the user chooses which QC method to run. The operator then chooses how many sets are needed. Then, in some embodiments, the application auto-selects the correct number of SNPs based on priority and prints output (picklist). If a picklist needs to be regenerated, the operator inputs which picklist they are replacing as well as which sets are not valid. The system auto-selects the valid SNPs plus replacement SNPs and print output. Additionally, in some embodiments, picklists are manually generated by SNP number.

The auto-selected SNPs are then removed from being listed as available for auto-selection. In some embodiments, the software prints the following items: SNP/Oligo list (picklist), SNP/Oligo layout (rack setup). The operator then takes the picklist into inventory and removes the completed oligonucleotide sets. In some embodiments, a completed set is unavailable. In this case, the operator regenerates a picklist. Then, in preferred embodiments, the missing SNP set or tube is flagged in the system. Once a picklist is full, the oligonucleotides are moved to the next step.

In some embodiments, the operator then takes the rack setup generated by the picklist and loads the rack. Alternatively, a robotic handling system loads the rack. In preferred embodiments, tubes are scanned as they are placed onto the rack. The scan checks to make sure it is the correct tube and displays the location in the rack where the tube is to be placed.

Completed racks are then placed-in a holding area to await the robot prep and robot run. Then, in some embodiments, the operator views what racks are in the queue and determines what genomics and reagent stock will be loaded onto the robot. The robot is then programmed to perform a specific method. Additionally, in some embodiments, the robot or operator records genomics and reagents lot numbers.

In preferred embodiments, a carousel location map is printed that outlines where racks are to be placed. The operator then loads the robot carousel according to the method layout. The rack is scanned (e.g., by the operator or by the ADS program). If the rack is not valid for the current robot method, the operator will be informed. The carousel location for the rack is then displayed. The output plates are then scanned (e.g., by the operator or by the ADS program). If the plate is not valid for the current method the operator is informed. The carousel location for the plate is then displayed.

Then, in some embodiments, the robot is run. The robot then places the plates onto heatblocks for a period of time specified in the method. In some embodiments, the robot then scans the plates on the Cytofluor. Output from the cytofluor is read into the database and attached to the output plate record.

In other embodiments, the output is read on the ABI 7700 real time instrument. In some embodiments, the operator loads the plate on to the 7700. Alternatively, in other embodiments, the robot loads the plate onto the ABI 7700. A scan is then started using the 7700 software. When the scan is completed the output file is saved onto a computer hard drive. The operator then starts the application and scans in the plate bar code. The software instructs the user to browse to the saved output file. The software then reads the file into the database and deletes the file (or tells the operator to delete the file).

The plate reader results (e.g., from a Cytofluor or a ABI 7700) are then analyzed (e.g., by a software program or by the operator). The present invention provides assessment methods to determine if a particular detection assay will pass the quality control component. The assessment process reviews the performance of the manufactured components (oligos: probe, invader, synthetic targets and CLEAVASE enzyme) of the detection assay (e.g., INVADER Assay, TAQMAN assay, etc.) under conditions similar, if not identical, to those that will be used by the customer. This automated process produces an assessment result (“PASS” or “FAIL”) and instructions as to the disposition (e.g. keep, reorder, resynthesize, bin) of the component oligonucleotides (ODNs) (e.g., probes, invader, targets) comprising the Assay. The latter role, the automated production of ODN disposition instructions, is an integral part of the overall modular and automated ODN production process due to the numerous platforms and configurations under which the INVADER Assay can be utilized.

This is achieved, for example, by testing an assay against several target types or classes, such as: No Target, Synthetic Target and Genomic Target. Utilizing these classes allows for the assessment process to be broken down into modules allowing for the numerous data and derived performance metrics to be funneled into an overall singular Pass/Fail code with the corresponding instructions for the disposition of the assay components.

This process may be employed, for example, for the assessment of the ODN components comprising the INVADER Assay. However, the assessment process may also be applied to the assessment of other assays (e.g. TAQMAN) and the ODN components that comprise other types of detection assays.

The assessment process of the present invention may be carried out in a series of steps.

Step 1—Assay Format

The assay format is based on the number of targets within each class is to be tested as well as the number of repetitions to which each target will be subjected.

Step 2—Allele Call Process

The general process for step 2 is outlined in FIG. 97A. In the case of a biplex assay, an allele call identification may be made by analyzing the raw data to derive three performance metrics, the FOZ (fold over zero) (calculated per signal dye/allele), and a FOZ Ratio. These metrics are compared to minimal threshold levels for making a genotyping call (Heterozygous, Homozygous_{WT}, Homozygous_{Mut}, or Equivocal/Ambiguous). If the two FOZ values can make a genotyping call that agrees with one made by the FOZ Ratio then the allele call is validated. Both validated calls and invalidated calls are then coded.

Performance Metrics

Performance metrics are those values that are mathematically derived from the raw data. The raw data is that generated by the device/instrument used to measure the assay performance (real-time or endpoint mode).

FOZ or S/NT
FOZ_{Dye1}=(RawSignal_{Dye1}/NTC_{Dye1})
FOZ_{Dye2}=(RawSignal_{Dye2}/NTC_{Dye2})
In the case of replicated runs, RawSignal_{DyeX} and NTC_{DyeX} are the averaged values.

FOZ Ratio
FOZ Ratio=(1−FOZ_{Dye1})/(1−FOZ_{Dye2})

CV
Coefficient of Variance=StDev_{signal}/Avg_{signal}
Performance Codes

Performance codes are those values that are generated based on the comparison of the aforementioned performance metrics to threshold metric values. This codification step not only sets the minimal metric value that can be used for making allele calls, but it also codifies why a specific well's performance metric failed.

Step 3—Class Analysis

The general process for step 3 is outlined in FIG. 97B. Allele Calls, both valid and invalid are grouped according to the target class, either genomic or synthetic. Each well's calls are then'sorted into two cases, valid and invalid calls.

Case 1: Valid Calls

Valid calls are simply tallied as either Homozygous (WT or Mut) and Heterozygous. Note that depending on the assay format/formulation, a Heterozygous call for synthetic targets may be deemed an invalid call.

Case 2: Invalid Calls

Invalid calls are those in which the genotype called using FOZs do not agree with those called using the FOZ Ratio method. Invalid calls may then be analyzed, depending on what target class, using a Failure Metrix that identifies the failing component ODN.

A Class Analysis Code is then generated by tallying the number of valid calls, sorted by genotype, and invalid calls, sorted by component ODN failure.

Step 4—Class Pass/Fail Flag

The general procedure for step 4 is outlined in FIG. 97C. The Class Analysis Codes are used and screened against a set of pass/fail/retest criteria which include:

Minimum number of Valid Calls—unambiguous or equivocal calls count against this number.

Allele representation—P/F/R (Pass/Fail/Retest) for the target class is based on a minimum number of Valid Homozygous calls for each allele that must be present in the tested target population.

Reproducibility—as reflected in the threshold CV value.

Step 5—SNP P/F/R

The general procedure for step 5 is presented in FIG. 97D. The status of the current SNP component ODNs is determined by the comparison/classification of the determined Class P/F/R Flag and the Class Analysis Codes. Weighting of one class over the other may be varied and is dependent upon the QC specification per customer and/or format. Recommendations as to the overall failure status of a particular component ODN may change depending on the result of another target Class Analysis Code and Class P/F/R Flag. A final SNP PFCode is issued which includes the total number of valid calls and the number of times a component ODN was deemed a failure.

Step 6—Component ODN Disposition

The general procedure for step 6 (and step 5) is presented in FIG. 97D. Depending on the result of the SNP PFCode the current SNP component ODN package is classified into the categories:

PASS

The component ODNs are all marked for shipment and the recommendation is forwarded to the appropriate production module.

FAIL

Instructions as to the disposition of each of the component ODNs are determined from the SNP PFCode. An action code is issued and is sent to the to appropriate production modules for processing (resynthesis/reorder).

RETEST

The component ODNs are saved and returned to the queue for retesting (not resythesized or reordered)

In some embodiments, the operator reviews the results of the software analysis of each SNP and takes one of several actions. In some embodiments, the operator approves all automated actions. In other embodiments, the operator reviews and approves individual actions. In some embodiments, the operator marks actions as needing additional review. Alternatively, in other embodiments, the operator passes on reviewing anything. Additionally, in some embodiments, the operator overrides all automated actions.

Depending on the results of the QC analysis, one of several actions is next taken. If the software marks ready for Full Fill, the operator forwards discards diluted Probe/INVADER oligonucleotide mixes and forwards the samples to the packaging module.

If an oligonucleotide set fails quality control, the data is interpreted to determine the cause of the failure. The course of action is determined by such data interpretation. If the software marks an oligonucleotide Reassess Failed Oligonucleotide, no action by user is required, the reassess is handled by automation. In the software marks an oligonucleotide Redilute Failed Oligonucleotide, the operator discards diluted tubes. No other action is required. If the software marks an oligonucleotide Order Target Oligonucleotide, no action by user is required. In this case, a synthetic target oligonucleotide is ordered for further testing. If the software marks an oligonucleotide Fail Oligo(s) Discard Oligo(s), the operator discards the diluted tubes and un-diluted tubes. No other action is required. If the software marks an oligonucleotide Fail SNP, the operator discards the diluted and un-diluted tubes. No other action is required. If the software marks an oligonucleotide Full SNP Redesign, the operator discards the diluted and un-diluted tubes. No other action is required. If the software marks an oligonucleotide Partial SNP Redesign the operator discards diluted tubes and discards some un-diluted tubes. No other action is required.

In some embodiments, the software marks an oligonucleotide Manual Intervention. This step occurs if the operator or software has determined the SNP requires manual attention. This step puts the SNP “on hold” in the tracking system while the operator investigates the source of the failure.

When a set of oligonucleotides (e.g., a INVADER assay set) is completed, the set is transferred to the packaging station.

In some embodiments of the present invention, the produced detection assays are tested against a plurality of samples representing two or more different alleles (samples containing sequences from individuals with different ethnic backgrounds, disease states, etc.) to demonstrate the viability of the assay with different individuals. In preferred embodiments, the produced assays are tested against a sufficient number of alleles (e.g., 100 or more) to identify which members of the population can be tested by the assay and to identify the allele frequency in the population of the genotype for which the assay is designed. In some embodiments, where certain individuals or classes of individuals are not detected by the detection assay, the target sequence of the individuals is characterized to determine whether the intended SNP is not present and/or whether additional mutations are present the prevent the proper detection of the sample. Any such information may be collected and stored in databases. In some embodiments, target selection, in silico analysis, and oligonucleotide design are repeated to generate assays capable of detecting the corresponding sequence of these individuals, as desired. In some embodiments, allele frequency information is stored in a database and made available to users of the detection assays upon request (e.g., made available over a communication network).

C. Packaging Component

In some embodiments, one or more components generated using the system of the present invention are packaged using any suitable means. In some embodiments, the packaging system is automated. In some embodiments, the packaging component is controlled by the centralized control network of the present invention.

D. Centralized Control Network

In some embodiments, the automated DNA production process further comprises a centralized control system. In some embodiments, the centralized control system comprises a computer system. In preferred embodiments, the centralized control system is operably linked to data (enterprise) management system (See, below). FIG. 58a-58k shows how the centralized control network if configured in some embodiments of the present invention.

In preferred embodiments, the centralized control network (for oligonucleotide processing) is operably linked to the centralized control system (for oligonucleotide synthesis). The combination of the centralized control system and centralized control network is referred to as the shop floor control system.

In some embodiments, the computer system comprises computer memory or a computer memory device and a computer processor. In some embodiments, the computer memory (or computer memory device) and computer processor are part of the same computer. In other embodiments, the computer memory device or computer memory are located on one computer and the computer processor is located on a different computer. In some embodiments, the computer memory is connected to the computer processor through the Internet or World Wide Web. In some embodiments, the computer memory is on a computer readable medium (e.g., floppy disk, hard disk, compact disk, DVD, etc). In other embodiments, the computer memory (or computer memory device) and computer processor are connected via a local network or intranet. In certain embodiments, the computer system comprises a computer memory device, a computer processor, an interactive device (e.g., keyboard, mouse, voice recognition system), and a display system (e.g., monitor, speaker system, etc.).

In preferred embodiments, the systems and methods of the present invention comprise a centralized control system, wherein the centralized control system comprises a computer tracking system. As discussed above, the items to be manufactured (e.g. oligonucleotide probes, targets, etc) are subjected to a number of processing steps (e.g. synthesis, purification, quality control, etc). Also as discussed above, various components of a single order (e.g. one type of SNP detection kit) may be manufactured in separate tubes, and may be subjected to a different number of processing steps. Consequently, the present invention provides systems and methods for tracking the location and status of the items to be manufactured such that multiple components of a single order can be separately manufactured and brought back together at the appropriate time. The tracking system and methods of the present invention also allow for increased quality control and production efficiency.

In some embodiments, the computer tracking system comprises a central processing unit (CPU) and a central database. The central database is the central repository of information about manufacturing orders that are received (e.g. SNP sequence to be detected, final dilution requirements, etc), as well as manufacturing orders that have been processed (e.g. processed by software applications that determine optimal nucleic acid sequences, and applications that assign unique identifiers to orders). Manufacturing orders that have been processed may generate, for example, the number and types of oligonucleotides that need to be manufactured (e.g. probe, INVADER oligonucleotide, synthetic target), and the unique identifier associated with the entire order as well as unique identifiers for each component of an order (e.g. probe, INVADER oligonucleotide, etc). In certain embodiments, the components of an order proceed through the manufacturing process in containers that have been labeled with unique identifiers (e.g. bar coded test tubes, color coded test tubes, etc.).

In certain embodiments, the computer tracking system further comprises one or more scanning units capable of reading the unique identifier associated with each labeled container. In some embodiments, the scanning units are portable (e.g. hand held scanner employed by an operator to scan a labeled container). In other embodiments, the scanning units are stationary (e.g. built into each module). In some embodiments, at least one scanning unit is portable and at least one scanning unit is stationary (e.g. hand held human implemented device).

Stationary scanning units may, for example, collect information from the unique identifier on a labeled container (i.e. the labeled container is ‘red’) as it passes through part of one of the production modules. For example, a rack of 100 labeled containers may pass from the purification module to the dilute and fill module on a conveyor belt or other transport means, and the 100 labeled containers may be read by the stationary scanning unit. Likewise, a portable scanning unit may be employed to collect the information from the labeled containers as they pass from one production module to the next, or at different points within a production module. The scanning units may also be employed, for example, to determine the identity of a labeled container that has been tested (e.g. concentration of sample inside container is tested and the identity of the container is determined).

The scanning units are capable of transmitting the information they collect from the labeled containers to a central database. The scanning units may be linked to a central database via wires, or the information may be transmitted to the central database. The central database collects and processes this information such that the location and status of individual orders and components of orders can be tracked (e.g. information about when the order is likely to complete the manufacturing process may be obtained from the system). The central database also collects information from any type of sample analysis performed within each module (e.g. concentration measurements made during dilute and fill module). This sample analysis is correlated with the unique identifiers on each labeled container such that the status of each labeled container is determined. This allows labeled containers that are unsatisfactory to be removed from the production process (e.g. information from the central database is communicated to robotic or human container handlers to remove the unsatisfactory sample). Likewise, containers that are automatically removed from the production process as unsatisfactory may be identified, and this information communicated to a central database (e.g. to update the status of an order, allow a re-order to be generated, etc). Allowing unsatisfactory samples to be removed prevents unnecessary manufacturing steps, and allows the production of a replacement to begin as early as possible.

As mentioned above, the tracking system of the present invention allows the production of single orders that have multiple components that may proceed through different production modules, and/or that may be processed (at least in part) in separate containers. For example, an order may be for the production of an INVADER detection kit. An INVADER detection kit is composed of at least 2 components (the INVADER oligonucleotide, and the downstream probe), and generally includes a second downstream probe (e.g. for a different allele), and one or two synthetic targets so controls may be run (i.e. an INVADER kit may have 5 separate oligonucleotide sequences that need to be generated). The generation of separate sequences, in separate containers, generally necessitates that the tracking system track the location and status of each container, and direct the proper association of completed oligonucleotides into a single container or kit. Providing each container with a unique identifier corresponding to a single type of oligonucleotide (e.g. an INVADER oligonucleotide), and also corresponding to a single order (a SNP detection kit for diagnosing a certain SNP) allows separate,,high through-put manufacture of the various components of a kit without confusion as to what components belong with each kit.

Tracking the location and status of the components of a kit (e.g. a kit composed of 5 different oligonucleotides) has many advantages. For example, near the end of the purification module HPLC is employed, and a simple sample analysis may be employed on each sample in each container to determine if a sample is collected in each tube. If no sample is collected after HPLC is performed, the unique identifier on the container, in connection with the central database, identifies the type of sample that should have been produced (e.g. INVADER oligonucleotide) and a re-order is generated. Identification of this particular oligonucleotide allows the manufacturing process for this oligonucleotide to start over from the beginning (e.g. this order gets priority status over other orders to begin the manufacturing process again). Importantly, the other components of the order may continue the manufacturing process without being discarded as part of a defective order (e.g. the manufacturing process may continue for these oligonucleotides up to the point where the defective oligonucleotide is required). Likewise, additional manufacturing resources are not wasted on the defective component (i.e. additional reagents and time are not spent on this portion of the order in further manufacturing steps).

The unique identifier on each of the containers allows the various components of a given order to be grouped together at a step when this is required (likewise, there is no need to group the components of an order in the manufacturing process until it is required). For example, prior to the dilute and fill module, the various components of a single order may be grouped together such that the contents of the proper containers are combined in the proper fashion in the dilute and fill module. This identification and grouping also allows re-orders to ‘find’ the other components of a particular order. This type of grouping, for example, allows the automated mixing, in the dilute and fill stage, of the first and second downstream probes with the INVADER oligonucleotide, all from the same order. This helps prevent human errors in reading containers and accidentally providing probes intended for one SNP being labeled as specific for a different SNP (i.e. this helps prevent components of different kits from being accidentally mixed together). The identification of individual containers not only allows for the proper grouping of the various components of a single order, but also allows for an order to be customized for a particular customer (e.g. a certain concentration or buffer employed in the second dilute and fill procedure). Finally, containers with finished products in them (e.g. containers with probes, and containers with synthetic targets) need to be associated with each other so they are properly assayed in the quality control module, and packaged together as a single kit (otherwise, quality control and/or a final end-user may find false negative and false positives when attempting to test/use the kit). The ability to track the individual containers allows the components of a kit to be associated together by directing a robot or human operator what tubes belong together. Consequently, final kits are produced with the proper components. Therefore, the tracking systems and methods of the present invention allow high through-put production of kits with many components, while assuring quality production.

E. Inventory Control Component

In some embodiments, the present invention provides an inventory control component. In certain embodiments, the inventory control component comprises a computer system and one or more inventory components (e.g. cold storage facility, robotic assay component handling means, bar code scanners). In preferred embodiments, the computer system comprises enterprise application (e.g. ORACLE, PEOPLESOFT, BAAN, etc.) with a standard inventory control and material resource planning (MRP) software. In preferred embodiments, the inventory control system is configured to track and store (e.g. for weeks or months) detection assay components or full detection assays (e.g. all ready assembled into a kit). In some embodiments, the inventory control component handles (e.g. stores and retrieves when necessary) the detection assay components and detection assays by product number, or by product family, or by individual detection assay component.

In preferred embodiments, the inventory control component comprises a computer system operably linked to the other components (e.g. order entry components, detection assay centralized control network) such that inventory in the system can be tracked. This allows inventory to be displayed to a user placing an order, and allows the detection assay production component to be given real time instructions (e.g. a bill of material) to produce more detection assays (e.g. before inventory of particular assays or components becomes too low or falls to zero). Operably linking the inventory control component to the other systems of the present invention (see Data Management Systems in part IV below) allows raw materials to be ordered in a timely fashion facilitating effective supply chain management.

Also in preferred embodiments, the inventory control component comprises a cold storage area with coded (e.g. bar coded) detection assay components, and automated (e.g. robotic) storage and retrieval device. In some embodiments, the storage and retrieval device is configured to receive instructions (e.g. bill of material) from the computer system to store or retrieve various assay components, and assemble them into a desired detection assay. For example, the storage and retrieval device receive instructions to assemble the components of an INVADER assay. The device reads the codes on the various assay components stored in containers (e.g. on carousels) in the cold room to find the proper assay components (e.g. an INVADER oligonucleotide, a probe oligonucleotide, a FRET oligonucleotide, and a positive control target). In other embodiments, the components are stored and retrieved by location such that the containers do not need to be scanned (or they could be scanned to verify the correct assay component is selected). Once the storage and retrieval device obtains the desired components, they may be passed along to the Dilute and Fill component, or Packaging component for shipment to a customer.

F. Detection Assay Production Example

This Example describes the production of an INVADER assay kit for SNP detection using the automated DNA production system of the present invention.

1. Oligonucleotide Design

The sequence of the SNP to be detected is first submitted through the automated web-based user interface or through e-mail. The sequences are then transferred to the INVADER CREATOR software. The software designs the upstream INVADER oligonucleotide and downstream probe oligonucleotide. The sequences are returned to the user for inspection. At this point, the sequences are assigned a bar code and entered into the automated tracking system. The bar codes of the probe and INVADER oligonucleotide are linked so that their synthesis, analysis, and packaging can be coordinated.

2. Oligonucleotide Synthesis

Once the probe and INVADER oligonucleotide sequences have been designed, the sequences are transferred to the synthesis component. The bar codes are read and the sequences are logged into the synthesis module. Each module in this example consists of 14 MOSS EXPEDITE 16-channel DNA synthesizers (PE Biosystems, Foster City, Calif.), that prepare the primary probes, and two ABI 3900 48-Channel DNA synthesizers (PE Biosystems, Foster City, Calif.), that prepare the INVADER oligonucleotides. Synthesizing a set of two primary and INVADER probes is complete 3-4 hours. The instruments run 24 h/day. Following synthesis, the automating tracking system reads the bar codes and logs the oligonucleotides as having completed the synthesis module.

The synthesis room is equipped with centralized reagent delivery. Acetonitrile is supplied to the synthesizers through stainless steel tubing. De-blocking solution (DCA in toluene) is supplied through Teflon tubing. Tubing is designed to attach to the synthesizers without any modification of the synthesizers. The synthesis room is also equipped with an automated waste removal system. Waste containers are equipped with ventilation and contain sensors that trigger removal of waste through centralized tubing when the cache pots are full. Waste is piped to a centralized storage facility equipped with a blow out wall. The pressure in the synthesis instruments is controlled with argon supplied through a centralized system. The argon delivery system includes local tanks supplied from a centralized storage tank.

During synthesis, the efficiency of each step of the reaction is monitored. If an oligonucleotide fails the synthesis process, it is re-synthesized. The bar coding system scans the container of the oligonucleotide and marks it as being sent back for re-synthesis.

Following synthesis, the oligonucleotides are transported to the cleavage and deprotection station. At this stage, completed oligonucleotides are subjected to a final deprotection step and are cleaved from the solid support used for synthesis. The cleavage and deprotection may be performed manually or through automated robotics. The oligonucleotides are cleaved from the solid support used for synthesis by incubation with concentrated NaOH and collected. The deprotection step takes 12 hours. Following cleavage and deprotection, the bar code scanner scans the oligonucleotide tubes and logs them as having completed the cleavage and deprotection step.

3. Purification

Following synthesis and cleavage, probe oligonucleotides are further purified using IS HPLC. INVADER oligonucleotides are not purified, but instead proceed directly to desalting (see below).

HPLC is performed on instruments integrated into banks (modules) of 8. Each HPLC module consists of a Leap Technologies 8-port injector connected to 8 automated Beckman-Coulter HPLC instruments. The automatic Leap injector can handle four 96-well plates of cleaved and deprotected primary probes at a time. The Leap injector automatically loads a sample onto each of the 8 HPLCs.

Buffers for HPLC purification are produced by the automated buffer preparation system. The buffer prep system is in a general access area. Prepared buffer is then piped through the wall in to clean room (HEPA environment). The system includes large vat carboys that receive premeasured reagents and water for centralized buffer preparation. The buffers are piped from central prep to HPLCs. The conductivity of the solution in the circulation loop is monitored as a means of verifying both correct content and adequate mixing. The circulation lines are fitted with venturis for static mixing of the solutions; additional mixing occurs as solutions are circulated through the piping loop. The circulation lines are fitted with 0.05 μm filters for sterilization and removal of any residual particulates.

Each purified probe is collected into a 50-ml conical tube in a carrying case in the fraction collector. Collection is based on a set method, which is triggered by an absorbance rate change within a predetermined time window. The HPLC is run at a flow rate of 5-7.5 ml/min (the maximum rate of the pumps is 10 ml/min.) and each column is automatically washed before the injector loads the next sample. The gradient used is described in Tables 3 and 4 and takes 34 minutes to complete (including wash steps to prepare the column for the next sample). When the fraction collector is full of eluted probes, the tubes are transferred manually to customized racks for concentration in a Genevac centrifugal evaporator. The Genevac racks, containing dry oligonucleotide, are then transferred to the TECAN Nap 10 column handler for desalting.

4. Desalting

Following HPLC purification (probe oligonucleotides) or cleavage (INVADER oligonucleotides), oligonucleotides move to the desalting station. The dried oligonucleotides are resuspended in a small volume of water. Desalting steps are performed by a TECAN robot system. The racks used in Genevac centrifugation are also used in the desalting step, eliminating the need for transfer of tubes at this step. The racks are also designed to hold the different sizes of desalting columns, such as the NAP-5 and NAP-10 columns. The TECAN robot loads each oligonucleotide onto an individual NAP-5 or NAP-10 column, supplies the buffer, and collects the eluate.

5. Dilution

Following desalting, the oligonucleotides are transferred to the dilute and fill module for concentration normalization and dispenation. Each module consists of three automated probe dilution and normalization stations. Each station consists of a network-linked computer and a Biomek 2000 interfaced with a SPECTRAMAX spectrophotometer Model 190 or PLUS 384 (Molecular Devices Corp., Sunnyvale Calif.) in a HEPA-filtered environment.

The probe and INVADER oligonucleotides are transferred onto the Biomek 2000 deck and the sequence files are downloaded into the Biomek 2000. The Biomek 2000 automatically transfers a sample of each oligonucleotide to an optical plate, which the spectrophotometer reads to measure the A260 absorbance. Once the A260 has been determined, an Excel program integrated with the Biomek software uses the measured absorbance and the sequence information to calculate the concentration of each oligonucleotide. The software then prepares a dilution table for each oligonucleotide. The probe and INVADER oligonucleotide are each diluted by the Biomek to a concentration appropriate for their intended use. The instrument then combines and dispenses the probe and INVADER oligonucleotides into 1.5 ml microtubes for each SNP set. The completed set of oligonucleotides contains enough material for,5,000 SNP assays.

If an oligonucleotide fails the dilution step, it is first re-diluted. If it again fails dilution, the oligonucleotide is re-purified or returned for re-synthesis. The progress of the oligonucleotide through the dilution module is tracked by the bar coding system. Oligonucleotides that pass the dilution module are scanned as having completed dilution and are moved to the next module.

6. Quality Control

Before shipping, the SNP set is subjected to a quality control assay in a-SAGIAN CORE System (Beckman Coulter), which is read on a ABI 7700 real time fluorescence reader (PE Biosystems). The QC assay uses two no target blanks as negative controls and five untyped genomic samples as targets.

The quality control assay is performed in segments. In each segment, the operator or automated system performs the following steps: log on; select location; step specific activity; and log off. The ADS system is responsible for tracking tubes. If a tube is missing, existing ADS program routines will be used to discard/reorder/search for the tube.

In the first step, a picklist is generated. The list includes the identity of the SNPs that are being tested and the QC method chosen. The tubes containing the oligonucleotide are selected by the automated software and a copy of the picklist is printed. The tubes are removed from inventory by the operator and scanned with the bar code reader and being removed from inventory.

The operator or the automated system then takes the rack setup generated by the picklist and loads the rack. Tubes are scanned as they are placed onto the rack. The scan checks to make sure it is the correct tube and displays the location in the rack where the tube is to be placed. Completed racks are placed in a holding area to await the robot prep and robot run.

The operator or the automated system then chooses the genomics and reagent stock to be loaded onto the robot. The robot is programmed with the specific method for the SNP set generated. Lot numbers of the genomics and reagents are recorded. Racks are placed in the proper carousel location. After all the carousel locations have been loaded the robot is run.

Places are then incubated on the robot. The plates are placed onto heatblocks for a period of time specified in the method. The operator then takes the plate and loads it into the ABI 7700. A scan is started using the 7700 software. When the scan is completed the operator transfers the output file onto a Macintosh computer hard drive. The then starts the analysis application and scans in the plate bar code. The software instructs the operator to browse to the saved output file. The software then reads the file into the database and deletes the file.

The results of the QC assay are then analyzed. The operator scans plate in at workstation PC and reviews automated analysis. The automated actions are performed using a spreadsheet system. The automated spreadsheet program returns one of the following results:
1) Mark SNP Oligonucleotide ready for full fill (Operator discards diluted Probe/INVADER mixes. Requires no other action).
2) ReAssess Failed Oligonucleotide (Requires no action by operator, handled by automation).
3) Redilute Failed Oligonucleotide (Operator discards diluted tubes. Requires no other action).
4) Order Target Oligonucleotide (Requires no action by operator, handled by automation).
5) Fail Oligo(s) Discard Oligo(s) (Operator discards diluted tubes. Operator discards un-diluted tubes. Requires no other action).
6) Fail SNP (Operator discards diluted tubes. Operator discards un-diluted tubes. Requires no other action).
7) Full SNP Redesign (Operator discards diluted tubes. Operator discards un-diluted tubes. Requires no other action).
8) Partial SNP Redesign (Operator discards diluted tubes. Operator discards some un-diluted tubes. Requires no other action).
9) Manual Intervention (This step occurs if the operator or software has determined the SNP requires manual attention. This step puts the SNP “on hold” in the tracking system).

The operator then views each SNP analysis and either approves all automated actions, approves individual actions, marks actions as needing additional review, passes on reviewing anything, or over rides automated actions.

Once the SNP set has passed the QC analysis, the oligonucleotides are transferred to the packaging station.

In some embodiments, the produced detection assay is screened against a plurality of known sequences designed to represent one or more population groups, e.g., to determine the ability of the detection assay to detect the intended target among the diverse alleles found in the general population. In preferred embodiments, the frequency of occurrence of the SNP allele in each of the one or more population groups is determined using the produced detection assay. Data collected may be used to satisfy regulatory requirements, if the detection assay is to be used as a clinical product.

IV. Data Management System

The present invention provides data management systems that integrate many of the components and systems of the present invention (See, e.g. FIGS. 58, 61 and 62). The data management systems of the present invention comprises networked computer processors (e.g. a local intranet), databases, and software applications that allow information to be shared and updated through the entire detection assay production and data collection process. The data management system may be comprised of the systems and components detailed above and below, all of which may be operably connected. This allows for integrated order entry, order analysis, assay design, assay production, inventory control, order shipping, and customer tracking, order tracking, inventory tracking, inventory control, and a product procurement module (e.g. that organizes ordering supplies from outside the company, or from within the same company, especially when manufacturing facilities are remote from one another). The data management systems of the present invention also facilitate other aspects of the present invention since information is constantly generated, evaluated, and stored (e.g. the rate of development of ASRs and Clinical diagnostics is increased, See Product Development section below).

In yet another variant the system and method of the present invention provides a data feed that affects production of one or more oligonucleotide detection assays by the detection assay production component. Moreover, the detection assay production component, the shipping component, the shop floor control system, inventory control component and/or other components of the system can also receive the data feed from the web order entry component. In yet a further variant, the data feed may also be bi-directional or omni-directional between these various components of the system.

By way of example, the web order entry component data feed may provide input for routines that control and regulate the detection assay production component, the shipping component, the shop floor control system, inventory control component, other components of the system, and/or combinations thereof. In another aspect, there is a data feed from the detection assay production component, the shipping component, the shop floor control system, inventory control component, other components of the system, and/or combinations thereof to provide the consumer or other user information such as whether or not a detection assay is in stock, needs to be manufactured, lead times, shipping times, etc.

In other variants, the data feed comprises statistical information associated with one or more oligonucleotide detection assays. This statistical information can be created by various routines used by the system and methods from raw data obtained from the web order entry component, the detection assay production component, the shipping component, the shop floor control system, inventory control component, other components of the system, and/or combinations thereof. This information is then used in forecasting reagent supplies needed, and/or ordering other ingredients or components of the detection assays.

A generalized overview of certain embodiments of the data management systems of the present invention are provided in FIGS. 61 and 62. These figures show various computer systems, networks, and software applications of data management systems and how these components may be connected to facilitate the production of detection assays. These figures also show various components of the production facility, including certain production components, an inventory control system, and their relationship to order entry and processing components. FIGS. 61 and 62 also demonstrate how the various computer systems, networks, and applications of the enterprise computer system are operably connected to the production components.

Referring specifically to FIGS. 61 and 62, initially an order is entered into the data management system by a client. This order may be a paper order (e.g. a contract for a large volume of assays), or it may be an electronic order placed-through a web interface (e.g. INVADERCREATOR). Generally the order comprises a target sequence containing a SNP that a client wants to detect with a detection assay produced by the systems of the present invention. This sequence is entered into the system, which may come via a web order entry process when the data management system is operably linked to the world wide web. Preferably when oligonucleotides are ordered, a link to an accounting type database verifies that an active purchase order is in place to cover any assay development costs. Generally, a particular target is given a part number that is associated with the particular target to be detected. Then, as described below, an assay is designed for this target and tested, or multiple assays are designed and tested. Employing part numbers allows quick identification of which SNP is being detected (e.g. for future orders, and to quickly find where the SNP is located on a chromosome).

This target sequence is then analyzed. For example, this target sequence may already have a part number because it has previously been received by the systems of the present invention. In certain embodiments, this previously received target sequence skips target sequence analysis (e.g. in silico analysis, and assay design steps), and proceeds directly to job submit. In certain embodiments, target sequences that do not require analysis and assay design are marketed to clients at a reduced cost. Preferably, databases of the present invention have this information stored allowing newly entered sequences to be quickly searched. Also, the part number tracking of particular target SNPs allows information to be retrieved on how many assays have been designed for this target, and known confidence levels associated with each (which allows better and better assays to be developed for each target, and/or differential pricing for assays with different levels of confidence). For example, is a customer does not identify what SNP they are trying to identify, the assay design process will be run (potentially increasing the price of the assay) to validate the part number.

The part number validation process generally has three steps. First, once an order is received, the data management systems of the present invention determine if an assay has previously been designed for this SNP. Next, data is accessed (if available) that determines if the previously designed assay worked, and at what confidence level. Finally, a determination is made if there was ever a re-design of the assay, and if there is a master assay that has been designed (e.g. one that has been shown to work, and shown to work with an acceptable confidence level).

In circumstances where the sequence that is received does not match previously received target sequences (e.g. it is a custom order), the systems of the present invention may be configured to extensively analyze the target sequences for suitability. This process, known as in silico analysis involves three general steps. First, a preliminary screening step is performed that screens out repeat sequences, as well as artifacts such a vector sequences. Then, a database search is performed with the candidate target sequence to determine if the candidate sequence corresponds to a known sequence, contains a unique SNP to be detected, and that results from such detection are known to be reliable. Finally, this information if processed and/or stored. This information may be used to report the candidate target sequence as a “high probability sequence” (will allow the production of a valid detection assay), and this information provided to the client, or used to move the sequence along the data management system to a detection assay design step. Processing of this information may also reveal one or more problems with the candidate target sequence allowing a report to be sent (e.g. by the internet) to a user (e.g., the person who input or requested the candidate target sequence or a technician utilizing the systems and methods of the present invention) highlighting the one or more problems.

If the target sequence is identified as a high probability sequence, or if the client requests that an assay be designed despite one or more problems, the target sequence information is forwarded (along the data management system) to the detection assay design systems of the present invention (e.g. comprising software applications to design assay components). In FIGS. 61 and 62, the detection assay design stage is represented with a long rectangular box containing “R-IC” (RNA INVADER CREATOR); “S-IC” (SNP INVADER CREATOR); “T-IC” (Transgene INVADER CREATOR); and “P-IC” (Primer INVADER CREATOR), as well as the design review box; Preferably, the data management system of the present invention has software applications for designing the components of a detection assay. These software applications process the target sequence and generate appropriate designs for detection assays (e.g. INVADER assays, TaqMan Assays, multiplexed primers, etc.).

FIGS. 61 and 62 provide examples of software applications useful designing INVADER assays, and PCR primers for any type of detection assay. For example, S-IC (SNP INVADER CREATOR) is an example of software application that generates the preferred DNA probes (with appropriate flap), and INVADER oligonucleotides (See, A.II.B). Also, P-IC “Primer INVADER CREATOR) is an example of a software application able to generate highly multiplexed sets of PCR primers to be used in conjunction with other detection assays. Once appropriate designs are generated, these designs are moved (e.g. along the enterprise computer system) to the “job submit” stage. The job submit stage may be a database of assays that need to be fulfilled. As shown in FIGS. 61 and 62, these assays may already be in inventory, or may have to be produced (at least in part) by the production facility. Since the data management systems of the present invention integrate various components allows, production and or inventory systems to be automatically activated (e.g. provided the correct instructions to begin assay production or to retrieve from storage, etc.).

If it is determined that the order can be filled from existing inventory, then many of the above steps may be skipped, and the order fulfilled from inventory. However, if it is determined that oligonucleotides need to be produced, the detection assay design is forwarded along the data management system (e.g. a work order or pick bill is generated) to the centralized control network that is operably connected to various production facility components (e.g. synthesis, cleave and deprotect) such that production is initiated.

Production may then begin with the oligonucleotide synthesis component. In preferred embodiments, more assays or components are generated than the work order actually requires (e.g. if one assay is ordered, ten are produced such that nine of the assays remain in inventory). In other preferred embodiments, the data management systems keep track of how many of each type of assays are produced and adjusts how many assays are made for inventory (e.g. keeping track of orders from individual customers or groups of customers allows forecasting of future orders, which may require that 20 assays are produced, instead of 10 assays, when inventory is depleted). In particular, instructions from the Centralized Control Network are sent to various oligonucleotide synthesizers. The oligonucleotide synthesis component produces requested oligonucleotides, which are then transferred to the oligonucleotide processing components (e.g. cleavage and deprotection component, oligonucleotide purification, dilute and fill, quality control, and shipping or inventory control components; see FIGS. 61 and 62). Preferably the tube, vials, and racks containing the requested oligonucleotides are labeled (e.g. with bar codes) such that the location of the oligonucleotides may be communicated to the centralized control network (and thus to other parts of the data management systems). This continues tracking allows all parts of the data management system to know in real time the status of particular orders. This information may be communicated back to the user (e.g. through a web interface, to customer service representatives, and to sales and business people), used to order raw materials, and used for business purposes.

Also information from the production facility, as shown in FIGS. 61 and 62, may be communicated to the inventory control component. Preferably the inventory control component, as noted above, not only contains physical storage of previously manufactured oligonucleotides and assay (e.g. labeled with bar codes), but also comprises Enterprise Resource Planning (ERP) software having a standard MRP inventory control system. Any type of enterprise software may be employed (e.g. ORACLE, SAP, PEOPLESOFT, BAAN, etc.).

In certain embodiments, the data management system, when linked to the world wide web, provides additional information back to a user who is using the allele caller function. For example, an allele call may be made for a particular assay and this information provided to the user via the web. Also sent with the allele information may be links to information on public databases (e.g. papers on the clinical relevance of this particular SNP, unpublished clinical association studies, or links to internet pages describing certain drugs available for treatment of any disease associated with the SNP, or number of assays for this target remaining in inventory, or price discounts for this customer for re-order, other relevant products available, etc.). In certain embodiments, the information returned to the user associates a patient ID number with the allele call test result (e.g. sent via the web to a computer or a personal digital assistant). In preferred embodiments, the client ID number has medical history information associated with it such that allele calls help determine what SNPs are associated with a particular medical condition.

In certain embodiments, the data management system is operably linked to a customer's computer or computer system (e.g. via the world wide web). In this regard, the systems of the present invention may periodically (or continuously) query a customers computer system to determine if the customer requires additional detection assays to be shipped. For example, the data management system of the present invention may query a customer's computer (e.g. a database on the customer's computer or computer system) to determine if inventory is running low or is exhausted for any particular type of detection assay. Also, the customer's detection equipment may provide data to the customer's computer (e.g. the customer is running an allele caller on their computer). This data may also be queried by the systems of the present invention such that detection assays may be automatically ordered, or a prompt may be sent informing the customer of the availability of certain detection assays. For example, it the data generated by a customer that is stored on the customer's computer indicates that the customer will likely require certain panels of detection assays be designed, the systems of the present invention may communicate the availability of such assays (e.g. via email) to the customer. In this regard, the present invention provides a commercial advantage by allowing customer specific detection assays (and panels of assay) to be offered and/or sent to the customer in an automated fashion. This provides convenience and ease of use for the customer, and increased sales for supplies of assays. The detection assay may be any type of detection assay, including INVADER assays and TAQMAN assays. If additional assay are needed, the systems of the present invention may automatically design different/different assays for a customer, and suggestions for what the customer may want to order. For example, an email may be sent letting the customer know that their inventory is running low, or that their previously generated results will logically lead to further orders for additional assays. The system of the present invention may also design additional assays (e.g. TAQMAN or INVADER assays), or suggest alternative assays to the user (e.g. suggest an INVADER assay replace the TAQMAN assay previously employed by the user).

In preferred embodiments, the customer/user is part of the medical community (e.g. physician or lab using detection assays to provide results to physician). In some embodiments, the computer system is in a physician's office. A customer (e.g. physician) may have results of detection assay use sent to his or her computer (e.g. from the customer's detection equipment or from an outside lab). This information may be queried by the systems of the present invention, which, as explained above, sends suggestions, alternative assays designs, or automatically sends detection assays. In further embodiments, information about what type of prescriptions a patient may require (e.g. based on the detection assay results) are provided to the physician (e.g. links to pages to order drugs that may required). In preferred embodiments, the detection assay reader device is located in the physician's office, and has a cost of less than ten thousand dollars. In preferred embodiments the patient's medical records are also used by the systems of the present invention to provide suggestions of prescriptions, and to suggest further detection assays that should be ordered (e.g. to avoid adverse drug reactions).

In certain embodiments, an electronic version of the Physicians Desk Reference (PDR), herein incorporated by reference, is available over the Internet. In preferred embodiments, the PDR may be queried by a user who is researching a particular condition. Preferably, the condition being queried by a user has information, or embedded information, that provides a user with particular detection assays that may be useful in diagnosing a disease, or confirming a disease, or to help avoid Adverse Drug Reactions with commonly prescribed medications. Preferably, the information regarding detection assays is operably linked to the Data Management Systems of the present invention. In this regard, one using the electronic PDR may be directed to an order screen to order the particular detection assays that may be required by the customer's patients.

V. Detection Assay Use and Data Generation and Collection

While the above sections describe the generation of a detection assay and the validation of the assay against a number of samples (e.g., several hundred samples), to fully investigate the viability of the detection assay against a broader population it is sometimes desired to conduct widespread testing with the detection assay. Where many different detection assays (e.g., hundreds to thousands of detection assays designed to identify unique markers) are to be investigated to facilitate moving products from research markets to clinical markets, large numbers of detection assays are tested against large numbers of samples.

In some embodiments, a detection assay producer distributes detection assays to research collaborators, whereby the research collaborators each conduct large numbers of tests (e.g., because of the inability of any one party to carry out a sufficient number of tests). The data generated by these tests (e.g. returned to the data management system via the web) is used to validate the detection assay (e.g., for use in obtaining regulatory approval). Test results may show that the detection assay is suitable or not suitable for use in certain population sub-sets. The test results may also show that detection assays, for whatever reason (e.g., for determined or undetermined scientific reasons), are not suitable for one or more testing markets (e.g., do not provide the requisite data to achieve regulatory approval). Where tests are determined not suitable for a desired market, new tests may be generated using the methods described above to identify a candidate test that meets the desired criteria.

Information generated through use of detection assays may be collected and fed back into the data management system of the present invention. In this regard, ASRs and Clinical diagnostic products may be quickly identified. In some embodiments, the detection assays are shipped to a customer with an agreement that assay results will be reported back (e.g. thus reducing the price of the product, or automatically reported back through detection instruments linked to the world wide web).

In some embodiments, a detection assay directed to a single target is used. However, in certain preferred embodiments, panels containing a plurality of different detection assays are employed (e.g., produced and used in testing). For example, panels containing two or more markers associated with a particular medical condition are employed. In some preferred embodiments, the panels contain thousands of unique markers, corresponding to every identified medically relevant marker.

The present invention provides systems and methods to provide researchers using the detection assays with information to assist in data collection as well as system and methods to collect and analyze data. In particularly preferred embodiments, collected data is automatically directed to a processor for analysis, storage, and compilation (e.g., compilation to support an application requesting regulatory approval of clinical products).

In some such embodiments, the present invention provides users with a means to find known information (including but not limited to information gleaned from public sources, publications, patents, and information previously determined by any user of the database) about any SNP, other mutations, or other sequence characteristic that has been entered a database. In some embodiments, the present invention provides a facile means of linking known and collected information about a particular SNP, other mutations, or other sequence characteristic to a particular test (e.g., assay test) of a sample. The utility of such applications is illustrated below for embodiments where SNP information is to be analyzed.

A. Association Databases

When a SNP has been linked to any other item of information (e.g., disease state, chromosome location, gene, ethnic group, allele frequency, another SNP), it can be considered to have an association. Association databases may be configured with reference to any association or combination of associations. In a preferred embodiment, an association database is configured to contain information about SNPs that have been determined to have medical relevance (i.e., to be relevant to'some aspect of health, including but not limited to the presence of disease, disease susceptibility and prognosis, and individual response to particular therapy).

In one embodiment, information about a SNP can be provided in a database table (e.g., a Microsoft Access database) having alphanumeric fields to provide details such as the gene identification, medical relevancy of the polymorphism, and literature or other references for the information provided (FIG. 63). Any number of fields are contemplated. In some embodiments, information may be as simple as a single gene name or an accession number in a database (e.g., GenBank). In other embodiments, the fields may provide more information, including but not limited to chromosome number, nucleotide, gene name, gene name abbreviation, genotype designation, allele location, GenBank accession number, NCBI URL link, dbSNP number, TSC number, targeted DNA sequence, disease category, disease association(s), SNP association(s) (i.e., other SNPs or mutations found to be associated the SNP being reviewed), patent status (e.g., whether a patent relating to that SNP. has been identified), patent number(s), and the NCBI OMIM database URL link. Additional links or items of information may be provided, such as links to online reference libraries and patent or other intellectual property databases. Disease categories may include, for example, metabolism, endocrinology, pulminology, nephrology, gastroenterology, neurology, genetic disease, musculoskeletal, and immunology. Additional categories may be designated to specifically identify diseases that overlap into two or more particular categories. Yet another kind of category may be provided (e.g., a “miscellaneous” category) for SNPs that have unknown or indeterminate association, that have a known association that does not fall within another category, or that, for any other reason, are not appropriately assigned to another category. In some embodiments the database has one field. In preferred embodiments the database has at least 10 fields, and in a particularly preferred embodiment, the database has at least 20 fields. In some embodiments, the database table is displayed on a screen (FIG. 63). In preferred embodiments, the screen is printable. In some embodiments, the fields are exportable to a spreadsheet file or worksheet (e.g., in Microsoft Excel; FIG. 64).

In one embodiment, the database may be searchable. In a preferred embodiment, the database is searchable, and is also configured to allow the user to present the resulting search data sets in an easily understandable, meaningful manner. In some embodiments, the database comprises an “allele caller” function, a function that provides allele calls (i.e., identification of the alleles detected in a given assay) based on the data input (e.g., such as from a fluorescent reader or mass spectrometer).

In some embodiments, the present invention provides a means for easily linking known information about a particular SNP to a particular test result on a sample through a “plate 4 viewer” format corresponding to the layout of samples in a reaction vessel or plate (FIG. 65). In preferred embodiments, the present information provides a means to use particular SNP test results on a sample to amend or update information about that SNP in an association database.

The following discussion provides one example of how a user interface for an association database may be configured. The user opens a work screen by clicking on an icon on a desktop display of a computer (e.g., a Windows desktop). The work screen features a menu (e.g., a drop down menu or “options” buttons) that allows the user to choose from available options. For example, in one embodiment, a user may be presented with the options of: 1) searching an association database; or 2) opening a plate viewer (as described above). In other embodiments, the user may have further or different options, such as 3) running an allele caller function. An option for exiting the program may be provided on the menu, as well. Examples of possible embodiments of user interfaces for each of these options are described, below.

1. Searching an Association Database:

In one embodiment, selecting this option opens a form having boxes that allow the user to make alphanumeric entries, and/or combination boxes (e.g., boxes that allow the user to either select from a list or make an alphanumeric entry) for each field represented in that particular association database. The user can enter search criteria in any field or set of fields. Upon clicking a “search” button, the program constructs a query, searching for record sets that include the specified strings in the corresponding fields.

Matching records from the search are assembled into sets. In some embodiments, the matching sets are displayed on a screen. In other embodiments, the matching sets are exported (e.g., sent to a printer or a file, or to a further process step) without display. In a preferred embodiment, the matching sets are displayed in a printable window.

In some embodiments, the user may select an entry from the matching set and view the information in the fields. In some embodiments, selection of an entry creates a display of the fields for that entry (FIG. 66). In preferred embodiments, the fields are displayed in a new window. In other embodiments, the fields are exported (e.g., sent to a printer or a file, or to a further process step) without display. In a preferred embodiment, the fields are displayed in a printable window. In some embodiments, one or more fields contain one or more local or Internet links (e.g., hypertext links or URLs). In preferred embodiments, SNPs listed in a SNP association field provide links to the record(s) of the associated SNPs. In particularly preferred embodiments, the user can click on links to bring up the corresponding content.

2) Using a Plate Viewer

As noted above, the present invention provides a means for easily linking known information about a particular SNP to a particular test result on a sample through a “plate viewer” format, i.e., in a fashion that corresponds to (e.g., visually represents) the layout of samples in a reaction vessel (FIG. 65). For example, if test assays for SNPs are performed in 96-well microtiter plates, which are arranged in grids of 8 wells×12 wells, the links to the information,regarding the SNPs would be displayed in a grid of 8×12 cells, such that each cell corresponds to the particular well in the plate (i.e., the test SNP in the 3^{rd} well of the 4^{th} row will have a link to its information presented on screen in the 3^{rd} cell of the 4^{th} row). Similar displays corresponding to other layouts of reaction vessels are contemplated (e.g., staggered grids, or circular or linear layouts). Any layout that can be replicated as a computer display is contemplated, including any non-gridded, or random distribution of reaction vessels in any arrangement that may be captured for representation on a computer display. Locations may be entered manually, or they may be automatically sensed and entered by methods such as digital imaging, coordinate sensing (e.g., such as that used for touch-screen computer displays), and the like.

Using a 384-well plate, a user selecting a “Plate Viewer” option should be presented with a table in the 384-well plate layout. In one embodiment, the SNPs entered into each cell of the table are assigned by the user (e.g., by entering identifying information from a particular field, such as a dbSNP number, into a selected cell on the plate viewer table). In preferred embodiments, SNPs are pre-assigned to particular cells. In particularly preferred embodiments, the SNPs are pre-assigned to cells in the table such that they correspond with an assay plate configured to test those SNPs in the corresponding wells. In other particularly preferred embodiments, the user selects from a menu of Plate Viewers, each having a different set of SNPs in pre-assigned cells corresponding with an assay plate configured to test those SNPs in the corresponding wells.

In one embodiment, the user selects.which field of the SNP record assigned to that cell will be displayed in the cell. In some embodiments, different fields from each SNP record may be displayed in each of the different cells: In other embodiments, the cells are coordinated so that the same field from each SNP record is displayed in each assigned cell. In a preferred embodiment, the user can globally change the fields displayed in all cells (e.g., through the use of a menu), such that all of the cells can be changed at one time to display the same field from each different SNP record.

In some embodiments, there is a code to visually distinguish test SNPs from control reactions (e.g., ‘no target’ controls or other controls). In preferred embodiments, the code is a color code.

In some embodiments, the user may select an entry from a cell and view (e.g., in a “data viewer”) the information in all of the fields for that SNP record (FIG. 66). In some embodiments, selection of an entry creates a display of the fields for that entry. In preferred embodiments, the fields are displayed in a new window. In other embodiments, the fields are exported (e.g., sent to a printer or a file, or to a further process step) without display. In a preferred embodiment, the fields are displayed in a printable window. In some embodiments, one or more fields contain one or more local or Internet links (e.g., hypertext links or URLs). In preferred embodiments, the user can click on links to bring up the corresponding content.

In some embodiments, an association database is provided on removable storage media (e.g., compact disc). In further embodiments, the storage media having the database includes an index of any PlateViewers having pre-assigned SNP records contained thereon. In preferred embodiments, the storage media having the database provides an indication of the currency of lo the information in the recorded database (e.g., a date or date range, version number, etc.). In preferred embodiments, the storage media having the database provides contact information for technical support (e.g., phone numbers facsimile numbers, email addresses, street addresses, names of technical support personnel, etc.).

B). Running an Allele Caller Function.

In some embodiments, the association database comprises an “allele caller” function, a function that provides identification of the alleles detected in a given assay, based on input assay data (e.g., from an instrument such as a fluorescent reader, nucleic. acid chip reader, or mass spectrometer).

The data to be processed by an allele caller may be provided in many different forms. In some embodiments, the data is raw signal, such as number corresponding to a measurement of fluorescence signal from a spot on a chip or a reaction vessel, or a number corresponding to measurement of a peak (e.g., peak height or area, as from, for example, a mass spectrometer, HPLC or capillary separation device). In some embodiments the data is imported directly from a measuring device. In other embodiments, the data is imported from a file. Raw data may be generated by any number of SNP detection methods, including but not limited to those listed below.

1. Direct Sequencing Assays

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

2. PCR Assay

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

3. Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

a. RFLP Assay

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are generally separated by gel electrophoresis and may be visualized by ethidium bromide staining. The length of the fragments is compared to molecular weight markers and fragments generated from wild-type and mutant controls.

b. CFLP Assay

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. In preferred embodiments, one or both strands are labeled. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by denaturing gel electrophoresis) and visualized (e.g., by autoradiography, fluorescence imaging or staining). The length of the fragments is compared to molecular weight markers and fragments generated from wild-type and mutant controls.

4. Hybridization Assays

In preferred embodiments of the present invention, variant sequences are detected a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., a,SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected;

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses-miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

C. Enzymatic Detection of Hybridization

In some embodiments of the present invention, hybridization is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with a fluorescent dye that is quenched by a second dye or other quenching moiety. Upon cleavage, the de-quenched dye-labeled product may be detected using a standard fluorescence plate reader, or an instrument configured to collect fluorescence data during the course of the reaction (i.e., a “real-time” fluorescence detector, such as an ABI 7700 Sequence Detection System, Applied Biosystems, Foster City, Calif.).

The INVADER assay detects specific mutations and SNPs in unamplified genomic DNA., In an embodiment of the INVADER assay used for detecting SNPs in genomic DNA, two oligonucleotides (a primary probe specific either for a SNP/mutation or wild type sequence, and an INVADER oligonucleotide) hybridize in tandem to the genomic DNA to form an overlapping structure. A structure-specific nuclease enzyme recognizes this overlapping structure and cleaves the primary probe. In a secondary reaction, cleaved primary probe combines with a fluorescence-labeled secondary probe to create another overlapping structure that is cleaved by the enzyme. The initial and secondary reactions can run concurrently in the same vessel. Cleavage of the secondary probe is detected by using a fluorescence detector, as described above. The signal of the test sample may be compared to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systemrs called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labelled antibody specific for biotin).

5. Other Detection Assays

Additional detection assays that are produced and utilized using the systems and methods of the present invention include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci USA 88, 189-93 (1991)); and sandwich hybridization methods (e.g., U.S. Pat. No. 5,288,609, herein incorporated by reference in its entirety).

6. Mass Spectroscopy Assay

In some embodiments, a MassARRAY system (Sequenom, San Diego, Calif.) is used to detect variant sequences (See e.g., U.S. Pat. Nos. 6,043,031; 5,777,324; and 5,605,798; each of which is herein incorporated by reference). DNA is isolated from blood samples using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

In some embodiments, data generated by different detection methods are processed to facilitate comparison, e.g., using an process like the Extraction-Transformation-Load paradigm from Data Warehousing, wherein data is “published” into a single repository, normalizing disparate data, and optimizing it for browsing and easy access to normalized, integrated data (e.g., DataMart and MetaSymphony software, NetGenics, Inc., Cleveland Ohio; U.S. Pat. No. 6,125,383, incorporated herein by reference in its entirety). SNP data generated by one SNP analysis method may be compared to SNP results data generated by another SNP analysis method (e.g., INVADER assay results are compared to gene chip data).

In some embodiments of the present invention, data is processed using an algorithm selected to determine an allele from the input assay data. The algorithm selected for processing data may be determined by the nature of the input assay data. The following provides an example of the application of an allele caller to an assay run in a microtiter plate (e.g., a 384-well plate).

The user enters information to identify the plate to be analyzed. In one embodiment, the plate may be identified by entry of a code number (e.g., a barcode number, part number, lot number). In another embodiment, the program provides a menu from which the user selects the number corresponding to the plate.

In some embodiments, the program provides a validation of the plate. For example, in some embodiments, the program verifies that the plate is of a suitable format for available analysis (e.g., that it corresponds to an assay for which an allele caller function can be provided). In other embodiments, the program verifies that the plate has been passed through some other process step. In some embodiments wherein the association database is provided on removable media (e.g., as described above), the program verifies that the version of the CD in use is suitable (e.g., has an appropriate version of an allele caller function, or has an appropriate association database) for use with the plate to be analyzed.

When a plate has been identified and determined to be valid for analysis, a record is displayed. In preferred embodiments, the record is a table having cells that correspond to assay wells on a microtiter plate (e.g., a “plate viewer”, described above). In some embodiments, the user has the option (e.g., through a menu selection) of creating a new analysis record or of calling up a record of a prior analysis. In preferred embodiments, the record links to identifying data from other analyses performed on the same collection of samples (e.g., name, date generated, etc.). In particularly preferred embodiments, SNP test wells on a plate are linked through a “plate viewer” function to SNP records in a database. In further particularly preferred embodiments, the database is an association database.

Prior to analysis, the assay data from the plate is imported, or “loaded” into the analysis program. It is contemplated that the data to be processed by an allele caller may be provided in many different forms. In some embodiments, the assay data is raw (i.e., unanalyzed) signal, such as a number corresponding to a measurement of fluorescence signal from a spot on a chip or a reaction vessel, or a number corresponding to measurement of a peak (e.g., peak height or area, as from, for example, a mass spectrometer, HPLC or capillary separation device). In some embodiments the data is imported directly from a measuring device. In other embodiments, the data is imported from a file. Raw assay data may be generated by any number of SNP detection methods, including but not limited to those listed above.

In some embodiments, the loaded assay data is displayed on a screen. In preferred embodiments, data is displayed in a plate viewer format. In some preferred embodiments, the layout is displayed in a new window. In particularly preferred embodiments, the window is printable.

Loaded assay data is then analyzed or processed using one or more algorithms selected to determine an allele from the input assay data. The algorithm selected for processing data is generally determined by the nature of the input assay data. In some embodiments, analysis involves determining the presence or absence of a signal (e.g., detectable fluorescence, or a detectable peak). In other embodiments, analysis involves determining the presence of a signal meeting a threshold value. In still other embodiments, analysis involves a comparison of more than one signal (e.g., examining differences in signal level, calculating ratios, etc.). In preferred embodiments, a SNP result (i.e., a determination of genotype at that locus, such as homozygous Allele 1 or Allele 2, heterozygous, Indeterminate) is determined when the processed data yields or corresponds to a value that has been predetermined to be indicative of a particular SNP result.

In some embodiments, the SNP results data from one plate are compared with the SNP results data from another plate. In other embodiments, SNP results data generated by one SNP analysis source method are compared to SNP results data generated by another SNP analysis method (e.g., INVADER assay results are compared to gene chip data).

In some embodiments, analysis results are displayed. In other embodiments, the analysis results are exported (e.g., sent to a printer or a file, or to a further process step) without display. In preferred embodiments, SNP results are displayed on a screen. In particularly preferred embodiments, results are displayed in a plate viewer (FIGS. 67 and 68). In some preferred embodiments, the plate viewer is displayed in a new window. In particularly preferred embodiments, the window is printable.

In some embodiments, the user may select a particular SNP result from the display of results and view the information in fields. In some embodiments, selection of an entry creates a display of the fields for that entry. In some embodiments, all the fields of the SNP record in an association database are shown. In other embodiments, a subset of the fields is shown. In preferred embodiments, fields in SNP results records include but are not limited to results of the analysis (e.g., homozygous Allele 1 or Allele 2, heterozygous, Indeterminate), the entered or imported raw input assay data (e.g., measured fluorescence, measured peaks, etc.), or the analyzed input assay data by which the allele determination was made (e.g., calculated differences in signal level, calculated ratios). In preferred embodiments, a field for user comments is included. In particularly preferred embodiments, the user comment field is editable after a SNP result has been obtained. In further particularly preferred embodiments, changes in a SNP result record may be saved by the user to that record or to a version of that record after a comment field is edited.

In some embodiments, the user selects which field of the SNP result record assigned to that cell will be displayed in the cell (FIGS. 67 and 68). In some embodiments, different fields from each SNP result record may be displayed in each of the different cells. In other embodiments, the cells are coordinated so that the same field from each SNP result record is displayed in each assigned cell. In a preferred embodiment, the user can globally change the fields displayed in all wells (e.g., through the use of a menu), such that all of the cells can be changed at one time to display the same field from each different SNP result record.

In preferred embodiments, the fields are displayed in a new window. In other embodiments, the fields are exported (e.g., sent to a printer or a file, or to a further process step) without display. In a preferred embodiment, the fields are displayed in a printable window. In some embodiments, one or more fields will contain one ore more local or Internet links (e.g., hypertext links or URLs). In preferred embodiments, the user can click on links to bring up the corresponding content.

In some embodiments, there is a code to visually distinguish test SNPs results and control reaction results (e.g., ‘no target’ controls or other controls). In preferred embodiments, the-code is a color code.

In some embodiments, the fields are exportable to a spreadsheet file or worksheet (e.g., in Microsoft Excel, FIG. 69). In some embodiments, SNP result data are exported to a worksheet by field content (e.g., one worksheet with all allele calls, one worksheet with all calculated ratios of signals, one worksheet with all raw input fluorescence measurements). In other embodiments, SNP results data are exported, all data is exported to a single worksheet, with data grouped according to the well with which it corresponds. In preferred embodiments, the user has the option (e.g., through a menu or window) of selecting a variety ways in which the SNP results data are sorted and/or grouped for export to a spreadsheet.

In preferred embodiments, following verification, assays for the detection of a given SNP are tested on a plurality of additional individuals. Data from additional assays is combined with information obtained from database searches. In preferred embodiments, the result is a revised reliability score for the SNP. In particularly preferred embodiments, data from additional analysis (e.g., results generated by an investigator using the methods and systems of the present invention) is used to update or amend an association database containing information about the given SNP.

C. Database Software

In some embodiments, GENOMICA (Boulder, Colo.) software is utilized to generate and host the SNP database of the present invention, which may be located, for example, on the data management systems of the present invention. In some embodiments, GENOMICA DISCOVERY MANAGER software is utilized. Genomica software utililizes Oracle databases to provide a web interface, security features, and reporting information (e.g., including but not limited to, the information described in Section C below). Depending on the particular application, one or more of the features of DISCOVERY MANAGER are utilized.

D. Revisions of Database Information

In preferred embodiments, the information (e.g., reliability scores) in the SNP database of the present invention is revised on a regular basis. In some embodiments, the revisions are automated. For example, users (e.g., customers) provide data from genotyping studies (e.g., through an automated web interface). In some embodiments, individual users are given a reliability rating based on the quality of their genotyping information. In preferred embodiments, the contribution to the reliability score of an individual's data is weighted based on the reliability rating of the user. In addition, individual databases are given reliability ratings based on the verification of their data.

E. Automated Genotyping

In preferred embodiments, the detection assays are employed in an automated or semi-automated fashion (e.g. a detection assay readout requires minimal human interaction), such that high throughput genotyping may be achieved. Any type of automated genotyping system of platform may be employed. In preferred embodiments, the automated genotyping systems of the present invention comprise at least one liquid handling platform, at least one detection platform, and at least one incubation component. Table 2 provides examples of such genotyping systems useful with the present invention.

TABLE 2

| System | Liquid Handler | Detection | Incubation | Robotics |
|---|---|---|---|---|
| CyBio | CyBi-well 384s (3) | TECAN Saffire | Liconic StoreX 200 | convey or rail |
| | | | or Heraeus 6070 | |
| Packard | 384 MPD (3) | TECAN SAFFIRE | Liconic StoreX 200 | convey or rail |
| Plate Track | | | or Heraeus 6070 | |
| Beckman | Biomek F/X | Perspective | Liconic StoreX 200 | OCRA 3M rail |
| CORE w/FX | (2 Arm-384) | Cytoflurs 4000 or | or Heraeus 6070 | |
| | | LJL Analyst | | |
| Packard | 384 MPD (1) | TECAN Saffire | Liconic StoreX 200 | convey or rail |
| Minitrack | | | or Heraeus 6070 | |
| CyBio | CyBi-Well | TECAN Saffire | Liconic StoreX 200 | convey or rail |
| | 384s (2) | | or Heraeus 6070 | |
| TECAN | TECAN Genesis | TECAN | Liconic StoreX | ROMA |
| workstation | 200 +/− M'mek (96) | Spectrofluor + | 44/200 | |
| Beckman | Biomek 200 +/− M'mek | Perspective | Liconic StoreX 44/ | ORCA 3M rail |
| CORE w/BK2 | (96) | Cytoflurs 4000 | 200 or Heraeus 6070 | |
| | | or LJL Analyst | | |

Other types of automated equipment and systems may be used with the systems of the present invention to facilitate high throughput genotyping. Other useful systems include Robbins, Cartesian, and Zymar systems. Exemplary liquid handling platforms include, but are not limited to; Beckman Coulter Biomek 200, Beckman Coulter Biomek FX, Beckman Coulter Multimek, CyBio CyBiWell 384, CyBio CyBiDrop, TECAN Genesis, 100, 150, 200 platforms, Cartesian Technologies SynQuad Systems, Zymark Sciclone ALH, Robbins Tango 384, Packard Multiprobe I and II, and Packard Mini & Plate Trak systems. Examplary detection platforms include, but are not limited to, Bio-Tek FL800,. Perseptive Cytofluor 4000, Tecan Genios, Tecan Spectrafluor Plus, PE Wallac Victor, BMG Fluorostar, Packard Fusion, Tecan Saffire, Tecan Ultra, LJL Analyst, and Packard Image Trak. Examplary Incubation components include, but are not limited to, manual incubation components including, but not limited to, Heat Blocks (e.g. 96 well plate), Thermalcyclers (e.g. used in incubator), Bio-Ovens (e.g. 10 plate), and Heraeus UT 6060 (e.g. 30 plate). Exemplary incubation components that are automation friendly include, but are not limited to, Liconic Store X 40 (e.g. 44 plate), Heraeus Cytomat 2 (e.g. 42 plate), Liconic StoreX 200 (e.g. 200 plate), and Heraeus Cytomat 6070 (e.g.189 plate).

An example of a protocol for set up of 96 and/or 384-well INVADER assays using the BIOMEK 2000 CORE system is shown in FIG. 59A. Also, FIGS. 59B, 59B, and 59C also show exemplary automated genotyping systems useful for high throughput screening. Further exemplary configurations for automated genotyping systems include, but are not limited to, the following five configurations: 1) System: Beckman Sagain CORE system, Robotics: Beckman Sagian 3m ORCA, Liquid Handler: Beckman Biomek 2000, Plate Washer Biomek 2000 WASH-8 tool, Incubation (75C):Dry Bath Heat Blocks, Incubation (60C): Heraeus Cytomat 6070 Automated Incubator, Reader: Perseptive Cytofluor 4000; 2) System: Beckman Sagian CORE system, Robotics: Beckman Sagian 3M ORCA, Liquid Handler: Beckman Biomek FX, Dual bridge with 96 and Span-8 channel pipettor heads, Plate Washer: Bio-Tek, Molecular Devices, etc., Incubation (75C): Liconic StoreX44 or Heraeus CytoMat2 Automated incubators, Incubator (60C): Liconic StoreX44 or Heraeus CytoMat2 Automated incubator, Reader: TECAN Safire, Spectrafluor, Ultra, or the like; 3) Robotics: Beckman Sagian, 2M Orca robot, Liquid Handler: Beckman Biomek FX, Dual bridge system with Span-8 and 384 pipette heads, Incubator: Heraeus Cytomat 6070, Reader: Tecan Safire Monochromator, Plate Storage: Beckman ambient carousel; 4) Robotics: Beckman Sagian, Coneyor Alps and onboard Gripper, Liquid Handler: Beckman FX, Dual bridge system with Span-8 and 384 pipette heads, Incubator: Heraeus Cytomat 6070, Reader; Tecan Safire Monochromator, Plate Storage: Heraeus Cytomat hotel (ambient); and 5) Robotics: Integral plate conveyors and rotating transfer arms, Liquid Handler: (3) CyBi Well 384 pipettors, and (1) CyBiDrop pipettor, Incubator: Liconic StoreX200, Reader: Tecan Safire Monochromator, and Plate Storage: CyBio high capacity plate stackers. Preferably, the automated genotyping systems of the present invention have a capacity of 50-75,000 genotypes per day in 384 well plates. In other preferred embodiments, the automated genotyping systems of the present invention have a capcity of at least 150,000, or at least 200,000 (e.g. aproximately 200,000) per day. It is understood that the automated genotying systems may require some off line plate arraying of either sample or probes to allow 384-chiannel pipetting and plate transfers to occur on the high throughput line.

F. Determination of Allele Frequencies in Pooled Samples

In particular embodiments, the present invention allows detection of polymorphims in pooled samples combined from many individuals in a population (e.g. 10, 50, 100, or 500 individuals), or from a single subject where the nucleic acid sequences are from a large number of cells that are assayed at once. In this regard, the present invention allows the frequency of rare mutations in pooled samples to be detected and an allele frequency for the population established. In some embodiments, this allele frequency may then be used to statistically analyze, the results of applying the INVADER detection assay to an individual's frequency for the polymorphism (e.g. determined using the INVADER assay). In this regard, mutations that rely on a percent of mutants found (e.g. loss of heterozygozity mutations) may be analyzed, and the severity of disease or progression of a disease determined (See, e.g. U.S. Pat. Nos. 6,146,828 and 6,203,993 to Lapidus, hereby incorporated by reference for all purposes, where genetic testing and statistical analysis are employed to find disease causing mutations or identify a patient sample as containing a disease causing mutations).

In some embodiments of the present invention, broad population screens are performed. In some preferred embodiments, pooling DNA from several hundred or a thousand individuals is optimal. In such a pool, for example, DNA from any one individual would not be detectable, and any detectable signal would provide a measure of frequency of the detected allele in a broader population. The amount of DNA to be used, for example, would be set not by the number of individuals in a pool, as was done in the 15-person pool described in Example 3, but rather by the allele frequency to be detected. For example, the assay in the 96-well format would give ample signal from 20 to 40 ng of DNA in a 90 minute reaction. At this level of sensitivity, analysis of 1 μg of DNA from a high-complexity pool would produce comparable signal from alleles present in only about 3-5% of the population. In some embodiments, reactions are configured to run in smaller volumes, such that less DNA is required for each analysis. In some preferred embodiments, reactions are performed in microwell plates (e.g., 384-well assay plates), and at least two alleles or loci are detected in each reaction well. In particularly preferred embodiments, the signals measured from each of said two or more alleles or loci in each well are compared.

### Pooled Sample

### EXAMPLE 1

This example describes the detection of a polymorphism in the APOC4 gene. In particular, this example describes the use of the INVADER assay to detect a mutation in the APOC4 gene in pooled samples.

In this example, genomic DNAs were isolated from blood samples from several individual donors, and were characterized by invasive cleavage for the T/C polymorphism in codon 96 of the APOC4 gene (See, Allan, et al., Genomics 1995 Jul 20;28(2):291-300, hereby incorporated by reference). The APOC4 assay used 5′ GATTCGAGGAACCAGGCCTTGGTGT (SEQ ID NO:1) 3′ as the invasive oligonucleotide and either 5′ ATGACGTGGCAGACAGCGGACCCAGGTCC-PO₄3′ (SEQ ID NO:2) or 5′ ATGACGTGGCAGACCGCGGACCCAGGTCC-PO₄3′ (SEQ ID NO:3) as primary signal probes for the T (Leu96) and the C (Pro96) alleles, respectively. The secondary target and probe were 5′ CGGAGGAAGCGTTAGTCTGCCACGTCAT-NH₂ 3′ (SEQ ID NO:4) and 5′ FAM-TAAC[Cy3]GCTTCCTGCCG 3′, respectively (SEQ ID NO:5).

All oligonucleotides were synthesized using standard phosphoramidite chemistries. Primary probe oligonucleotides were unlabeled. The FRET probes were labeled by the incorporation of Cy3 phosphoramidite and fluorescein phosphoramidite (Glen Research, Sterling, Va.). While designed for 5′ terminal use, the Cy3 phosphoramidite has an additional monomethoxy trityl (MMT) group on the dye that can be removed to allow further synthetic chain extension, resulting in an internal label with the dye bridging a gap in the sugar-phosphate backbone of the oligonucleotide. Amine or phosphate modifications, as indicated, were used on the 3′ ends of the primary probes and the secondary target oligonucleotides to prevent their use as invasive oligonucleotides. 2′-O-methyl bases in the secondary target oligonucleotides are indicated by underlining and were also used to minimize enzyme recognition of 3′ ends. Approximate probe melting temperatures (Tₘs) were calculated using the Oligo 5.0 software (National Biosciences, Plymouth, Minn.); non-complementary regions were excluded from the calculations.

Pooled samples were constructed by diluting the heterozygous (het) DNA into DNA that is homozygous T (L96) at this locus. The test reactions contained 0.08 to 8 μg of T (L96) genomic DNA per reaction, and the het DNA was held at 0.08 μg, thus creating a set of mixtures in which het DNA represented from 50% down to 1% of the total DNA in the sample (See, FIG. 70). The actual representation of the C (P96) allele ranged from 25% down to 0.5% of the copies of this gene in the mixed samples. Controls included reactions having either all T (L96) DNA at each of the various DNA levels, or all het DNA at the 80 ng level. In addition, a sample of DNA that is homozygous for the C (P96) allele was tested (FIG. 2).

For all the INVADER assay reactions, 4 pmol of invasive probe, 40 pmol of FRET probe, and 20 pmol of secondary target oligonucleotide were combined with genomic DNA in 34 μl of 10 mM MOPS (pH 7.5) with 1.6% PEG. Reactions with the C (Pro96) allele of the APOC4 gene contained 80 ng of DNA heterozygous for this allele, and included DNA homozygous for the T (Leu96) allele at the indicated ratios. Samples were overlaid with 15 Iul of Chill-Out liquid wax and heated to 95° C. for 5 min to denature the DNA. Upon cooling to 67° C. the reactions were started by the addition of 400 ng of Cleavase VIII enzyme, 15 pmol of either the T (Leu96) or the C (Pro96) primary signal probe, and MgCl₂ to a final concentration of 7.5 mM. The plates were incubated for 2 hours at 67° C., cooled to 54° C. to initiate the secondary (FRET) reaction, and incubated for another 2 hours. The reactions were then stopped by addition of 60 μl of TE. The fluorescence signals were measured on a Cytofluor fluorescence plate reader at excitation 485/20, emission 530/25, gain 65, temperature 25° C. Three replicates were done for each reaction and for no-target controls. The average signal for each target DNA was calculated, the average background from the no-target controls was subtracted, and the data plotted using Microsoft Excel.

The results of this example are shown in FIG. 70. As shown in this figure, the C (P96) allele was easily detected in all reactions, including that in which it was present in only 0.5% of the APOC4 alleles present in the mixture. These data indicate that the invasive cleavage reactions can be used for population analysis using pooled DNA samples. This has the double advantage of reducing the number of assays required to verify a new SNP, and of allowing the use of one large preparation of pooled DNA for numerous tests, thereby reducing the influence of sample-to-sample variations in DNA purity.

The above example demonstrates that the INVADER assay may be used to screen a population. A. sample of mixed DNA to be analyzed should be large enough to bring the low-frequency alleles into the detectable range, e.g., 80 to 100 ng of the variant genome in these 40 μl reactions. As shown above in this Example, a sample of 8 to 10 μg of mixed DNA allowed detection of alleles present at 0.5 to 1% of the population under these conditions. In addition, the DNA from any one individual ideally should not be present in a large enough quantity to generate a detectable signal when an aliquot of the pool is tested. Creating a pool of several hundred individuals should guarantee that any detected signal reflects a contribution from many individuals in the pool. Finally, the use of a second probe set as an internal standard would allow the signals to be normalized from reaction to reaction, and would allow the prevalence of any SNP to be measured more accurately.

### Pooled Sample

### EXAMPLE 2

This example describes the detection of a polymorphism in the CFTR gene. In particular, this example describes the use of the INVADER assay to detect the AF508 mutation in the CFTR gene in a pooled sample.

For INVADER assay analysis of the AF508 mutation, the primary probe set comprised 5′ ATATTCATAGGAAACACCAAG 3′ (SEQ ID NO:6) as the invasive oligonucleotide and either 5′ AACGAGGCGCACAGATGATATTTTCTTTAA 3′ (SEQ ID NO:7) or 5′ ATCGTCCGCCTCTGATATTTTCTTTAATGG 3′ (SEQ ID NO:8) as signal probes for the wild type and the mutant alleles. The secondary reaction components were designed to function optimally at a temperature at least 5 degrees below the primary reaction temperature.

All oligonucleotides described were synthesized using standard phosphoramidite chemistries. Primary probe oligonucleotides were unlabeled. The FRET probes were labeled by the incorporation of Cy3 phosphoramidite and fluorescein phosphoramidite (Glen Research, Sterling, Va.). While designed for 5′ terminal use, the Cy3 phosphoramidite has an additional monomethoxy trityl (MMT) group on the dye that can be removed to allow further synthetic chain extension, resulting in an internal label with the dye bridging a gap in the sugar-phosphate backbone of the oligonucleotide. One nucleotide was omitted at this position to accommodate the dye. Amine modifications were used on the 3′ ends of the primary probes, the secondary target and the arrestor oligonucleotides to prevent their use as invasive oligonucleotides. 2′-O-methyl bases are indicated by underlining and are also used to minimize enzyme recognition of 3′ ends. Approximate probe melting temperatures were calculated using the Oligo 5.0 software (National Biosciences, Plymouth, Minn.); noncomplementary regions were excluded from the calculations.

DNA samples characterized for CFTR genotype were purchased from Coriell Institute for Medical Research (Camden, N.J.), catalog numbers NA07469 (heterozygous in the CFTR gene for both AF508 and R553X mutations) and NA01531 (homozygous AF508). To determine what dose of a mutant could be detected within a pooled sample using the FRET-sequential invasive cleavage approach, DNA that is the heterozygous for the AF508 mutation in the CFTR gene was diluted into DNA that is homozygous wild type at that locus. The test reactions contained 0.1to 2.6 μg of the total genomic DNA per reaction, and the mutant DNA was held at 0.1 μg, thus creating a set of mixtures in which mutant DNA represented from 50% down to 4% of the total DNA in the sample. Because the mutant DNA was heterozygous at the 508 locus, the actual allelic representation ranged from 25% down to 2% of the DNA in the mixed samples. Controls included reactions having either all wt at each of the various DNA levels, or all heterozygous mutant DNA at the 100 ng level. In addition, a sample of DNA that is homozygous for the AF508 mutation was tested.

DNA concentrations were estimated using the PicoGreen method. 4 pmol of INVADER probe, 40 pmol of FRET probe, and 20 pmole of secondary target oligonucleotide were combined with genomic DNA in 34 μl of 10 mM MOPS (pH 7.5) with 4% PEG. Samples were overlaid with 15 μl of Chill-Out liquid wax and heated to 95° C. for 5 min to denature the DNA. Upon cooling to 62° C. the reactions were started by the addition of 400 ng of AfuFEN1 enzyme, 15 pmole of either wt or mutant primary probe, and MgCl₂ to a final concentration of 7.5 mM. The plates were incubated for 2 hours at 62° C., cooled to 54° C. to initiate the secondary (FRET) reaction, and incubated for another 2 hours. The reactions were then stopped by addition of 60 μl of TE. The fluorescence signals were measured on a Cytofluor fluorescence plate reader excitation 485/20, emission 530/25, gain 65, temperature 25° C. Three replicates were done for each reaction and for no-target controls. The average signal for each target DNA was calculated, the average background from the no-target controls was subtracted, and the data plotted using Microsoft Excel.

The results of this Example are presented in FIG. 71. Analysis of the signal from the mutant allele shows that it is not noticeably inhibited by substantial increases in the amount of wild type DNA, and the AF508 mutant DNA could be easily detected when present as only 2% of the mixture (FIG. 71). These data indicate that the invasive cleavage reactions can be used for population analysis using pooled DNA samples. This has the double benefit of reducing the number of assays required to verify a new SNP, and of allowing the use of one large, preparation of the pooled DNA to be used for numerous tests, thereby reducing the influence of sample-to-sample variations in DNA purity.

Application of the INVADER assay to screen populations is possible given the results presented in this example. In preferred embodiments for population screening, the DNA contribution from each individual should be equal, and the DNA from any one individual should not be present in a large enough quantity to generate a detectable signal when an aliquot of the pool is tested. For example, for this system creating a large enough pool that any one person contributes less than 1 ng (e.g., 0.5 ng) to each reaction should guarantee that any detected signal reflects a contribution from many individuals in the pool. For other detection systems, limiting the DNA from any one individual to an amount less than the detection limit of the system, for example ⅕ to 1/10 the detection limit, should produce the desired effect. The use of a second probe set as an internal standard, for example, would allow the signals to be normalized from reaction to reaction, and would allow the prevalence of any SNP to be measured more accurately.

### Pooled Sample

### EXAMPLE 3

This example describes the detection of the Consortium No. TSC 0006429 (SNP 1831) mutation in pooled samples. DNA from 15 individuals was purchased from the Coriell Cell Repository and each sample was tested to identify the genotype at the SNP Consortium No. TSC 0006429 (SNP 1831) locus. Each reaction contained 40 ng of DNA from each individual, 0.366 μM primary probe. 0.0366 μM Invader oligonucleotide, 0.183 μM FRET Probe and 100 ng CLEAVASE VIII enzyme in a buffer of 10 mM MOPS (pH 7.5) with 7.5 mM MgCl₂.

The probes used were as follows (5′ to 3′):

Invader:

(SEQ ID NO:9)

CTTACTTGACCTTGGGCCCAGTTATTTAACCTTCTAGACCT;
Probe T:

(SEQ ID NO:10)

CGCGCCGAGGATCAGTTTCTTCATCTCTAAAATGGA;
Probe G:

(SEQ ID NO:11)

CGCGCCGAGGCTCAGTTTCTTCATCTCTAAAATGGA;
Synthetic Target T:

(SEQ ID NO:12)

TGTATCCATTTTAGAGATGAAGAAACTGAG;

(SEQ ID NO:13)

GGTCTAGAAGGTTAAATAACTGGGCCCAAGGTCAAGTAAGGG;
Synthetic Target G:

(SEQ ID NO:14)

TGTATCCATTTTAGAGATGAAGAAACTGAT;

(SEQ ID NO:15)

GGTCTAGAAGGTTAAATAACTGGGCCCAAGGTCAAGTAAGGG

The assays were performed as described in Hall et al., PNAS, 97 (15):8272 (2000). Briefly, reaction were incubated at a constant temperature of 65° C. The data for each sample, produced using an ABI 7700 instrument for real-time reaction detection, are shown in the 15 panels of FIGS. 72 and 73, with signals from the G allele shown as the light line and from the T allele shown as the dark line. The signal from each allele present in the mixture appears as an ascending curve reflecting the quadratic nature of the signal accumulation; the signal from any allele not present is essentially a straight line. These DNAs were then pooled in several combinations: Samples 1-5, 6-10, 11-15, 1-10, 6-15, and 1-15. The data panels are shown in FIG. 74. FIG. 75 provides a comparison of the net fluorescence counts measured at the end of each reaction. From the results in 66a-b, the allele representation in each mixture can be calculated. Both FIGS. 74 and 75 demonstrate that the aggregate signals for each pool are proportional with respect to the final ratio of the alleles in the mix. The net fluorescence signals from the pooled samples are greater than those from the individuals because the amount of DNA from each person was held constant. For example, the assays run on DNA pooled from 5 individuals had 5 times as much DNA as the assays run on DNA from one individual.

As seen in this example, the real-time detection capabilities of the ABI 7700 can prove invaluable in detecting rare SNPs. Because the reaction is a two-step cascade, the real-time trace of signal accumulated in the Invader assay fits to a quadratic equation (i.e., the curves observed in FIGS. 72, 73, and 74), but background signal remains linear over the course of the reaction. Consequently, distinguishing signal arising from the genomic target from the background fluorescence is straightforward. This characteristic of the assay means that low-level signals from rare alleles can be resolved from background with more certainty.

### Pooled Sample

### EXAMPLE 4

Measurement of different alleles within a single reaction removes concerns about sample-to-sample variations introducing inaccuracies into the measurements to be compared in the determination of allele frequency. Use of biplex (detection of two alleles or loci per reaction) or more complex multiplex (detection of more than two alleles or loci per reaction) configurations increases the through-put for allele frequency determination and facilitates comparisons of allele frequencies between different populations (e.g., affected vs. non-affected with a particular trait).

The following provides one example of a general protocol for the detection of two alleles in a DNA sample, and several examples wherein the protocol has been applied to the determination of alleles in samples. In this example, the signals are measured from fluorescein dye (FAM) and REDMOND RED dye (Red, Synthetic Genetics, San Diego, Calif.), each used on a separate FRET probe in combination with the Z28 ECLIPSE quencher (Synthetic Genetics, San Diego, Calif.). This protocol is provided to serve as an example and is not intended to limit the use of the methods or compositions of the present invention to any particular assay protocol or reaction configuration. Numerous fluorescent dyes and fluorophore/quencher combinations, and the methods of attaching and detecting such agents alone and in FRET combinations to nucleic acids are known in the art. Such other agents combinations are contemplated for use in the present invention and their use in these methods is within the scope of the present invention.

a. Procedure for Allele Frequency Determination in Pooled DNA

1. Determine the DNA concentration of each of the samples to be used in the INVADER Assay using the PICOGREEN reagents (procedure follows).
2. Mix the DNA samples at the desired ratios to mimic pools of genomic samples at specified allelic frequencies.
3. Denature the genomic DNA samples by incubating them at 95° C. for 10 min. Sample may then be placed on ice (optional).
4. Prepare a Probe/INVADER oligonucleotide/MgCl₂ mix by combining the 1.15 μL probe/INVADER oligonucleotide mix (3.5 μM of each primary probe and 0.35 μM INVADER oligonucleotide) and the 1.85 μL 24 mM MgCl₂ per reaction. Preparation of a master mix sufficient for testing of the complete set of samples is preferred.
5. Add 3 μl of the appropriate control or sample DNA target at 80 to 100 ng/μl (approximately 240-300 ng of genomic DNA) to the appropriate well of a 384-well biplex INVADER Assay FRET detection plate (Third Wave Technologies, Madison, Wis.). Each plate well contains 3 μl of a solution, dried after dispensing, containing 10 mM MOPS, 8% PEG, 4% glycerol, 0.06% NP 40, 0.06% Tween 20, 12 ug/ml BSA, 50 ng/ul BSA, 33.3 ng/ul CLEAVASE VIII enzyme, 1.17 μM FAM FRET probe (5′-FAM-TCT (Z28) AG CCG GTT TTC CGG CTG AGA GTC TGC CAC GTC AT-3′, SEQ ID NO:16) and 1.17 μM Red FRET Probe (5′-Red-TCT (Z28) TC GGC CTT TTG GCC GAG AGA CCT CGG CGC G -3′, SEQ ID NO:17).
6. Next, pipette 3 μl of Probe/INVADER oligonucleotide/MgCl₂ mix into the appropriate wells of the 384-well biplex INVADER Assay FRET detection plate.
7. Overlay each reaction with 6 μL of mineral oil.
8. Cover the plates with an adhesive cover and spin at 1,000 rpm in a Beckman GS-15R centrifuge (or equivalent) for 10 seconds to force the probe and target into the bottom of the wells.
9. Incubate the reactions at 63° C. for 3-4 hours in a thermal cycler or incubator such as a BioOven III. After 3-4h incubation at 63° C., lower the temperature to 4° C. if a thermalcycler is being used or to RT if an incubator is being used.

10. Analyze the microtiter plate on a fluorescence plate reader using the following parameters:

Wavelength/Bandwidth

| | | |
|---|---|---|
| FAM: | Excitation: | 485 nm/20 nm |
| | Emission: | 530 nm/25 nm |
| Red: | Excitation: | 560 nm/20 nm |
| | Emission: | 620 nm/40 nm |

b. Calculation of Fold-over-zero Minus 1 (FOZ−1):

The signals from each reaction are measured by comparison to the signal from a no-target control (the ‘zero’) and are expressed as a multiple of the signal from the ‘zero’ reaction. The factor one is subtracted to get the factor of actual signal over the background (e.g., for a sample having 1.5× the signal of the zero or 1.5 fold-over-zero, the amount of specific signal is 1.5-1, or 0.5).

Determine FOZ−1 as follows:

FOZ−1 FAM Probe=((raw counts FAM probe 1, 485/530) (raw counts from No Target Control FAM probe, 485/530))−1.

FOZ−1 Red Probe=((raw counts Red probe 2, 560/620) (raw counts from No Target Control Red probe, 560/620))−1

c. Calculation the Correction Factor (CF) as Follows

A correction factor can be calculated to accommodate any variations in the efficiencies of the cleavage reactions between the probe sets.
CF_{FAM}=(FOZ_{FAM}−1)/(FOZ_{Red}−1); CF_{Red}=(FOZ_{Red}−1)/(FOZ_{FAM}−1) of a heterozygous control.

For the FAM allelic frequency calculation:

$$\frac{( {FOZ}_{FAM} - 1 ) / {CF}_{FAM} )}{( ( {FOZ}_{FAM} - 1 ) / {CF}_{FAM} ) + ( {FOZ}_{Red} - 1 )} \times 100$$

For the Red allelic frequency calculation:

$$\frac{( {FOZ}_{Red} - 1 ) / {CF}_{Red} )}{( ( {FOZ}_{Red} - 1 ) / {CF}_{Red} ) + ( {FOZ}_{FAM} - 1 )} \times 100$$

d. DNA Quantitation Procedure (Molecular Probes PICOGREEN Assay)

The PICOGREEN reagent is an asymmetrical cyanine dye (Molecular Probes, Eugene, Oreg.). Free dye does not fluoresce, but upon binding to dsDNA it exhibits a >1000-fold fluorescence enhancement. PICOGREEN is 10,000-fold more sensitive than UV absorbance methods, and highly selective for dsDNA over ssDNA and RNA.

1. Turn on the fluorescence plate reader at least 10 minutes before reading results. Use the following settings to read the PICOGREEN results:

Wavelength/Bandwidth

| | |
|---|---|
| Excitation | ˜485 nm/20 nm |
| Emission: | ˜530 nm/25 nm |

2. Prepare 1× TE buffer (10 mM Tris-HCl, 1 mM EDTA, pH 7.5) from the 20× TE stock which is supplied in the PICOGREEN kit (to make 50 ml, add 2.5 ml of 20× TE to 47.5 ml sterile, distilled DNase-free water). 50 ml is sufficient for 250 assays.
3. Dilute DNA standards from 100 μg/ml to 2 μg/ml with 1× TE. For two standard curves, prepare 400 μl of a 2 μg/ml stock by adding 8 μl of the 100 μg/ml stock to 392 μl 1× TE.

4. Prepare the two standard curves in the microtiter plate as shown in the table 7:

TABLE 7

| | Final | | Vol. (μl) |
|---|---|---|---|
| | [DNA] | Vol. (μl) 2 μg/ml | 1X TE |
| Plate Well | (ng/ml) | DNA Standard | Buffer |
| A1 & A2 | 0 | 0 | 100 |
| B1 & B2 | 25 | 2.5 | 97.5 |
| C1 & C2 | 50 | 5 | 95 |
| D1 & D2 | 100 | 10 | 90 |
| E1 & E2 | 200 | 20 | 80 |
| F1 & F2 | 300 | 30 | 70 |
| G1 & G2 | 400 | 40 | 60 |
| H1 & H2 | 500 | 50 | 50 |

5. For each unknown, add 2 μl of sample to 98 μl of 1× TE in the microplate well. Mix by pipetting up and down.
6. Prepare a 1:200 dilution of the PICOGREEN reagent in 1× TE. For each standard and each unknown sample, a volume of 100 μl is needed. For example, 2 standard curves with 8 points each will require 1.6 ml. To. calculate the total volume of diluted PICOGREEN reagent needed, determine the total number of samples and-unknowns will be tested and multiply this number by 100 μl (if using a multichannel pipet, make extra reagent). The PICOGREEN, reagent is light sensitive and should be kept wrapped in foil while thawing and-in the diluted state. Vortex well.
7. Add 100 μl of diluted PICOGREEN to every standard and sample. Mix by pipetting up and down.
8. Cover the microplate with foil and incubate at room temperature for 2-5 minutes.
9. Read the plate.
10. Generate a standard curve using the average values of the standards and determine the concentration of DNA in the unknown samples.
   e. Measurement of Allele Frequencies in Genomic DNA Samples

DNA samples having alleles at various frequencies were created by mixing different homozygous genomic DNA samples at different ratios. Each pool contained a total of 240 ng genomic DNA, and the reactions were carried out in 384-well plates as described- above, at 63° C. for 3 hours. The measured signals are shown in FIG. 76A. The allelic frequiencies were calculated based on the relative signal generated by the FAM and Red reporter dyes, and are displayed graphically in FIG. 76B. These data show the correlation between the theoretical or actual allelic frequency (the frequency intended to be created by mixing known amounts of DNA), compared to the allelic frequency calculated from the INVADER assay data.

An 8-way pool of the genomic DNA of different individual was also tested. Each of the 8 DNA was previously, characterized for each of 8 different SNP loci, so that the allelic frequency for each of the 8 SNPs in the pool was known. In this test, each pool contained a total of 300 ng genomic DNA, and the reactions were carried out in 384-well plates as described above, at 63° C. for 3 hours. The measured signals for the FAM channel, the rarer allele in each case, is shown in FIG. 77. The graph compares the known frequencies for each allele to the frequencies calculated from the INVADER assay data.

DNAs homozygous for each of two different SNPs (SNP132505 and SNP131534) were combined at various ratios to simulate genomic pools with different allelic frequencies. Each pool contained a total of 240 ng genomic DNA, and the reactions were carried out in 384-well plates as described above, at 63° C. for 3 hours. The allelic frequencies were calculated based on the relative signal generated by the FAM and Red reporter dyes, and are displayed graphically in FIGS. 78A and 78B.

The probes used in the tests described above and additional probes sets suitable for use in the methods of the invention are shown in FIG. 80A-C.

VI. Integrated Information, Design, and Production

Data gathered from the use of detection assays on one or more samples (e.g., as described in Section V, above) may be used to generate and expand powerful genomics databases and to supplement and improve target selections, detection assay design, detection assay productions, and detection assay use, and further analysis of detection assay results. The data may also be used to obtain regulatory approval for clinical products for detection assays that are demonstrated to meet the necessary requirements for clinical regulatory approval (described below). While, for clarity, each of the components of the systems and methods of the present invention have generally been described herein in isolation, each component relates to each other component, and the synergy between the components provides enhanced systems and methods for acquiring and analyzing biological information. This synergy, as it relates to some embodiments of the present invention, is represented in FIG. 81. The center of the figure shows genomic databases representing phenotypic databases (e.g., disease databases), genomic databases (e.g., genome sequence databases, polymorphism databases, allele frequency databases, etc.), and expressed RNA databases. Data in the databases is derived from any number of sources. For example, the databases may contain data from compiled public or private databases. Data may also be actively incorporated using systems and methods of the present invention. As shown in FIG. 81, data is received from investigators (e.g., using a communication network) providing target sequence requests for in silico analysis, detection assay design, and/or detection assay production (See e.g., Sections AI, AII, and AIII, above).

In some embodiments, new data is generated during the processes of the present invention (e.g, produced assays may be tested on a plurality of samples to determine allele frequencies, as described in Section AIII). New data is also received from detection assay data gathered from investigators (See e.g., Section AV, above). In some embodiments of the present invention, information is tracked and correlated from the initial target sequence requests to the final detection assay result data analysis.

Newly collected data may be incorporated into a number of aspects of the present invention. It can be used to refine in silico analysis, e.g., to provide improved output information; it may be added to an association database, e.g., to note newly observed associations within existing fields, and/or to define new fields indicating new types of associations, such as allele frequency within populations tested.

The following example is provided to illustrate certain preferred embodiments of the present invention. In this example, the systems for performing in silico analysis, detection assay design and production, and information management and analysis are provided by a service provider. Target sequences to be analyzed are provided by a first user (e.g., a researcher, pharmaceutical company, government agency, etc.) and detection assays generated to detect the target sequence are used by the first user and/or other users.

The first user selects a target sequence of interest. For example, an investigator may have identified a SNP in a human genomic sequence that is correlated to disease state (e.g., a SNP correlated to cardiovascular disease, diabetes, development of cancer, rare inherited disorders, asthma, neurological diseases, obesity, sexual dysfunction, hypertension, and the like). In some cases, the investigator will have identified the mutation and/or correlation in a very small population sample (e.g., in a single individual). The investigator may wish to determine the allele frequency of the SNP in the general population and may wish to generate an accurate diagnostic test to determine if an individual possesses the SNP, and is therefore at a higher risk than the general population of contracting or exhibiting the correlated disease or condition. In other embodiments, an investigator may have a SNP that is only suspected to correlate to a disease state, and may wish to generate an accurate diagnostic test to screen large numbers of individuals who have been assessed for the presence or absence of the disease state in order to determine the whether the suspected correlation in fact exists. In other cases, the investigator may wish to determine the frequency of an allele within one or more populations for purposes including assessing risk for correlated disease states in the one or more populations. To address these needs, the investigator employs the systems and methods of the present invention.

The investigator uses a computer system to access a computer system of the service provider. In some embodiments, the investigator simply uses a personal computer system to access a publicly available Web site of the service provider. As discussed in Section I, above, the user transmits the identified target-sequence containing the SNP to the computer system of the service provider. The target sequence is then processed through the in silico analysis systems and methods (Section I) and the detection assay design systems and methods (Section II) of the present invention. A report is sent to the investigator indicating any problems identified in the in silico analysis or design process and, in some embodiments, alternate target sequence suggestions are provided. The report may also indicate several options for the design of a detection assay from which the investigator may select. In some embodiments, at the time the original target sequence is submitted by the investigator, the investigator selects options for determining whether a report is provided (e.g., as opposed to simply proceeding with production without generating a report), the conditions under which a report is provided, and the information content of the report.

Once a target sequence is selected and design parameters for the detection assay components are selected (e.g., type of target [RNA or DNA] sequences of probes and primers, reaction temperatures, buffer conditions, etc.), information is passed to the production component of the systems and methods of the present invention (Section III). Production of the detection assay is carried out and quality control steps are used to ensure that the detection assay functions as intended (i.e., is capable of detecting the SNP in a sample). In some embodiments, the produced detection assay is screened against a plurality of known sequences designed to represent one or more population groups, e.g., to determine the ability of the detection assay to detect the intended target amongst the diverse alleles found in the general population. Produced assays are then shipped to detection assay users (e.g., the investigator who entered the target sequence and other investigators).

At each of the stages described above, information is tracked and stored. For example, the original target sequence request from the investigator is assigned a tracking number and information about the investigator (e.g., previous request information), information obtained from in silico analysis, information obtained from design analysis, and information obtained from production analysis (e.g., allele frequency information) is collected, correlated to the tracking number, and incorporated into the databases of the present invention. For example, allele frequency information is stored in a SNP allele frequency database, information obtained from in silico analysis and design analysis are stored for use in improved analysis of future target sequences, and information about investigators requesting the produced detection assays are stored and used to generate an information template for receiving detection assay data from the user after the assays are used (Section V). If in silico analysis determines that a SNP was previously characterized, the new request is assessed to see if it provides any additional information (e.g., additional information provided by the new user), and such new information is integrated into the existing records for that SNP in the databases (e.g., association databases, allele frequency databases). In some embodiments, the information about the target sequence and SNP obtained from the in silico, design, and production analysis are integrated with the information template to allow the investigator to access information (e.g., disease associations, allele frequency, etc.) prior to, during, or following use of the detection assay (e.g., information may be linked to a plate viewer function described in Section IV above).

The investigator uses the detection assay on one or more samples, e.g., as described in Section V, above. Information and data are collected and returned to the systems of the service provider. Information and data obtained by the service provider from use of the detection assay are used for obtaining regulatory approval of clinical products corresponding to successful detection assays and to supplement information databases and improve in silico analysis, assay design, assay production, and future information dissemination to investigators. For example, additional allele frequency information may be obtained from the investigator. This information is used to supplement allele frequency databases. This information may also be used to increase or decrease the number of samples used during production analysis of allele frequency, as certain samples (e.g., samples from particular ethnic groups, disease states, etc.) may be determined to be of limited information content (e.g., redundant) while others represent important, but previously unidentified or unappreciated populations for future analysis of allele frequency testing. Failure data from investigators (e.g., the failure of hybridization probes to hybridize to target sequences in a sample) is used in future in silico and design analysis.

As is clear from the above description, wide-scale use of the systems and methods of the present invention provides solutions to the unmet needs of the fields of bioinformatics and molecular diagnostics and medicine. Each phase of the invention, from target sequence validation and assay design and production to assay use and data collection provides a continuous circle of data generation and improvement. Wide scale use of the systems and methods of the present invention provides for the generation of reliable detection assays for the detection of any target sequence, wherein assays are designed to work for all individuals (e.g., a single assay that works for all individuals or a plurality ofassays, each working for a known sub-set of the population). Databases generated using the systems and methods of the present invention provide comprehensive information pertaining to the allele frequency of mutations in one or more populations and the correlations of sequences and gene expression patterns to phenotypes. Thus, in some embodiments, the present invention provides detection assays and corresponding information databases and analysis systems for accurately screening entire populations (e.g., screening all human newborns) for sequences and expression patterns corresponding phenotypes (e.g., disease states, drug responses, etc.). Using the databases of the present invention, a specific sequence, combination of sequences, or expression patterns in an individual may be correlated to proven responses appropriate for the individual (e.g., avoidance of allergens, therapeutic drug treatments, gene therapy, preventive routes or behaviors, etc.).

B. Development of Clinical Detection Assays

As discussed above, of the thousands of markers evaluated using the systems and methods of the present invention, a sub-set of the markers are reliably detected by the detection assays of the present invention. Where a detection assay is shown to reliably detect a marker (e.g., a medically-relevant marker), detections assays for use as analyte-specific reagents or clinical diagnostics are prepared. Analyte-specific reagents and clinical diagnostics are regulated in the United States. Using the systems and methods of the present invention, data generated during the development of the detection assays is used to support regulatory approval of the detection assay for used as ahalyte-specific reagents and clinical diagnostics. Because the present invention provides easy-to-use, efficient, accurate detection assays (e.g., the INVADER assay) that can be produced for thousands of unique markers at high production capacity and because the present invention provides systems and methods for widespread testing and data collection of thousands of samples with each of the thousands of unique detection assays, sufficient information is gathered to support regulatory approval of numerous clinical products. The present invention provides systems arid methods for testing all identified markers, selecting markers that are suitable for clinical use, and collecting data in support of regulatory approval for every clinically relevant marker. The specific regulatory requirements for analyte-specific reagents and in vitro diagnostics are outlined below.

A major class of markers and mutations that find use in diagnostics are drug metabolism enzymes. Drug-metabolizing enzymes (DMEs) help the body to break down drugs properly and enable their therapeutic effects. One or more variations in a DME gene may affect how a person responds to a particular drug. As a result, one person may respond positively to a drug, while another may suffer adverse reactions to the same drug and still another will be unaffected by it. Detection assays that detect DME mutations expand the markets of existing drugs and the revival of drugs not allowed to or removed from the market because of adverse drug reactions or lack of therapeutic effect. The use of the present invention also provides high throughput screening of prospective new drug compounds that can eliminate potentially toxic drug candidates from development early in the process; reduces the cost and risk of clinical drug trials through pre-trial genetic screening; and provides clinical diagnostics to determine appropriate drug and dosage before prescription to avoid adverse drug reactions. The present invention also provides screening methods for selecting drug therapy such that adverse drug reactions are avoided.

I. Adverse Drug Reactions and Genetic Variation

More than 3 billion prescriptions are written each year in the U.S. alone, effectively preventing or treating illness in hundreds of millions of people. But prescription medications alsoxcan cause powerful toxic effects in a patient. These effects are called adverse drug reactions (ADR). Adverse drug reactions can cause serious injury and or even death. Differences in the ways in which individuals utilize and eliminate drugs from their bodies are one of the most important causes of ADRs (MedWatch).

More than 106,000 Americans die—three times as many as are killed in automobile accidents—and an additional 2.1 million are seriously injured every year due to adverse drug reactions. ADRs are the fourth leading cause of death for Americans. Only heart disease, cancer and stroke cause more deaths each year. Seven percent of all hospital patients are affected by serious or fatal ADRs. More than two-thirds of all ADRs occur outside hospitals. Adverse drug reactions are a severe, common and growing cause of death, disability and resource consumption in North America and Europe.

ADRs most commonly occur when the body cannot change a drug quickly enough into a form that it can use and then eliminate. A drug compound goes through a series of many changes as it is being processed in the body, some of which actually may make the drug more toxic before it is changed again. If this toxic form of the drug is not changed or eliminated by the body, it can cause illness, permanent liver damage or even death. Proteins called drug-metabolizing enzymes (DMEs) make these changes as the body processes a drug.

All drugs have the potential to cause ADRs. The most common, however, are central nervous system agents (antidepressants, anticonvulsants, eye and ear preparations, internal analgesics and sedatives), anti-infectious drugs (penicillin and the sulfa antibiotics), anti-cancer drugs and cardiovascular drugs. Cardiovascular drugs alone cause 25 percent of all ADRs.

It is estimated that drug-related anomalies account for nearly 10 percent of all hospital admissions. Drug-related morbidity and mortality in the U.S. is estimated to cost from $76.6 to $136 billion annually.

A. Cytochrome p450 Polymorphisms

The cytochrome p450 (CYP) superfamily comprises a group of enzymes that play an essential role in the bio-transformation of medically relevant compomounds. Approximately 40% of CYP isoforms are polymorphic, including CYP1A2, 3A4, 2B6, 2CP, and 2C19 (see also Table 8 below). Accurate genotyping of patients for these and other p450 loci is important because allelic variants may lead to loss of efficacy or toxic accumulation.

TABLE 8

| Gene | Location | Substrate |
|---|---|---|
| CYP1AI | 15q22-q24 | Benzo(a)pyrene, phenacetin |
| CYP1A2 | 5q22-qter | Acetaminophen, amonafide, caffeine, paraxanthine, |
| | | ethoxyresorufin, propranolol, fluvoxamine |
| CYP1B1 | 2p21 | estrogen metabolites |
| CYP2A6 | 19q13.2 | Coumarin, nicotine, halothane |
| CYP2B6 | 19q13.2 | Cyclophosphamide, aflatoxin, mephenytoin |
| CYP2C19 | 10q24.1-24.3 | Mephenytoin, omeprazole, hexobarbital, mephobarbital, |
| | | propranolol, proguanil, phenytoin |
| CYP2C8 | 10cen-q26.11 | Retinoic acid, paclitaxel |
| CYP2C9 | 10q24 | Tolbutamide, warfarin, phenytoin, nonsteroidal anti- |
| | | inflammatories |
| CYP2D6 | 22q13.1 | Flexainide, guanoxan, methoxyamphetamine, N- |
| | | propylajmaline, perhexiline, phenacetin, phenformin, |
| | | propafenone, sparteine |
| CYP2E1 | 10q24.3-qter | N-Nitrosodimethylamine, acetaminophen, ethanol |
| CYP3A4/3A5/3A7 | 7q21.1 | Macrolides, cyclosprorin, tacrolimus, calcium channel |
| | | blockers, midazolam, terfenadine, lidocaine, dapsone, |
| | | quinidine, triazolam, etopside, teniposide, lovastatin, |
| | | tamoxifen, steroids, benzo(a)pyrene |

One example of a drug influenced by a CYP loci is the drug WARFARIN, which is a blood thinner routinely prescribed to prevent or treat blood clots, especially those associated with heart attack or heart value replacement and to reduce the risk of death, another heart attack or stroke after a heart attack. More than 19 million prescriptions for the drug were written in 2000. Approximately eight percent of whites and two percent of blacks have a genetic variation (CYP2C9*3) that causes the body to slow its metabolism of WARFARIN, which can cause bleeding that can resulting in the loss of large amounts of blood.

Genetic screening for this variation allows health care professionals to prescribe the correct dosage of WARFARIN to avoid the severe bleeding and to preclude the use of aspirin, which could further thin the blood and amplify the adverse reaction.

Many of the p450 genes are highly polymorphic. INVADER assays, and other detection assays, can be used to detect particular polymorphisms in p450 genes in order to help prevent adverse drug reactions in patients. One example is the CYP2D6 gene. FIG. 82 shows the various polymorphisms for this gene. Importantly, the two CYP2D pseudogenes, CYP2D7 and CYP2D8, share many of the identified polymorphisms of CYP2D, and over 80% sequence similarity. Therefore, to prevent false positive results, due to detection of the two psuedogenes, a CYP2D6 specific Triplex PCR amplification reaction was developed to integrate with the INVADER assay. The three PCR products are amplified from genomic template in a single tube using CYP2D6 specific PCR primers with a 35 cycle PCR reaction of 95 degrees Celsius for 20 seconds and 68 degrees Celsius for 2 minutes (see FIG. 83).

Next, a 1/20 dilution of the CYP2D6 specific PCR products are used as a template for polymorphism detection using the Biplex INVADER assay system in a single well of a 96 or 384 well plate. Two serial INVADER assay reactions occur simultaneously, target detection and allele discrimination takes place in the primary INVADER reaction, while signal amplification takes place in the secondary INVADER reaction using a set of universal signal probes. The entire assay is isothermal and only requires a single step to set up. In addition to this, signal can be read and alleles called after only 20 minutes incubation at 63 degrees Celsius following an initial 5 minutes 95 degrees Celsius denaturation step (See FIG. 84). The results of a screen of 175 individuals using this approach is shown in FIGS. 85 and 86.

i. CYP2D6

In some embodiments, the present invention provides methods of characterizing a cytochrome p450 allele comprising; a) providing i) a sample comprising at least Y target sequences, wherein each of the target sequences comprises at least a portion of a cytochrome p450, and wherein each of the target sequences comprises a footprint region, a 5′ region immediately upstream of the footprint region, and a 3′ region immediately downstream of the footprint region, ii) a primer set comprises a forward and a reverse primer sequence for each of the at least Y target sequences, iii) at least one assay probe configured to detect a footprint region, wherein the primer set is configured for performing a multiplex PCR reaction that amplifies at least Y amplicons, wherein each of the amplicons is defined by the position of the forward and reverse primers, b) amplifying the Y target sequences with the primer set; and c) detecting at least one of the footprint regions with the assay probe. In certain embodimetns, the at least one footprint region of the Y target sequences comprises a polymorphism. In additional embodiments, the polymorphism is a single nucleotide polymorphism, or a duplication or a deletion. In preferred embodiments, the cytochrome p450 gene comprises a CYP 2D6 allele.

### ii. CYP2D6 Examples

### Characterization of Cytochrome p450 2D6 Alleles using Triplex PCR and the INVADER Assay System

The field of pharmacogenetics is advancing rapidly as increasing numbers of functional polymorphisms in proteins essential for drug action are identified. One of the most clinically important of these proteins is an enzyme in the cytochrome P450 family, debrisoquine 4-hydroxylase, or cytochrome P450 2D6 (CYP2D6), the gene for which is found on chromosome band 22q13.1. This enzyme metabolizes about 25% of all therapeutic drugs, including beta-blockers, serotonin reuptake inhibitors, anti-emetics, tricyclic anti-depressants, anti-arrhythmics, and nicotine. In addition, CYP2D6 metabolizes many environmental xenobiotic substances. Hence, the metabolic status of the enzyme has been linked to a wide range of illnesses such as liver cancer (Agundez, J. A., et al. Lancet, 1995. 345(8953):830) and Parkinson's disease (Smith, C. A., et al. Lancet, 1992. 339(8806):1375).

Currently, more than 70 polymorphisms have been identified within the exonic and promoter regions of CYP2D6 ; an equal number of haplotypes (e.g., see the world wide web site at imm.ki.se/CYPalleles/cyp2d6.html) have also been identified. Numerous genetic variations (more than 20 polymorphisms, a gene deletion, and a number of gene conversion events) cause decreased CYP2D6 activity. Depending on ethno-geographic origins, the overall incidence of poor metabolizer (PM) status in the general population ranges between 1-8%, (Sachse, C., et al., Am J Hum Genet, 1997. 60(2):284). In addition, multiple copies of alleles of CYP2D6 have been associated with extensive (EM) and ultra-rapid (UM) metabolizers (Johansson, I., et al., Proc Natl Acad Sci U S A, 1993. 90(24): p. 11825; Lovie, R., et al., FEBS Lett, 1996. 392(1):30).

Directly upstream of the approximately 5-kb CYP2D6 gene lay two CYP2D6 pseudogenes, CYP2D7 and CYP2D8 (FIG. 123A). Both pseudogenes are highly homologous (97% and 92%, respectively) to the exonic sequence of the CYP2D6 gene (Kimura, S., et al., Am J Hum Genet, 1989. 45(6):889). Many rare alleles found in CYP2D6 also occur in CYP2D7 and CYP2D8. Despite the importance of the CYP2D6 enzyme in drug metabolism and adverse drug effects, the complexity in its genomic region has hampered attempts to develop clinical genetic tests for variations in this enzyme. Here we describe a simple, scalable, and comprehensive CYP2D6 genotyping strategy that combines selective amplification of the CYP2D6 gene with the specificity of the invasive signal amplification reaction, or INVADER reaction.

PCR-INVADER Assay Strategy

As described above, the CYP2D6 genomic region contains two adjacent pseudogenes, CYP2D7 and CYP2D8. To prevent false positive results or inflated wild type signals caused by INVADER oligonucleotides hybridizing to the pseudogenes, a series of PCR primers that specifically- allow amplification of only CYP2D6 was devised. This PCR product was then used as a target for the INVADER assay reaction.

The INVADER Assay Reaction.

The biplex format of the INVADER DNA assay enables simultaneous detection of two DNA sequences in a single well, such as two variants of a particular polymorphism; The biplex format uses two different allele-specific primary probes, each with a unique 5′ flap, and two different FRET cassettes, each with a spectrally distinct fluorophore. By design, the released 5′-flaps will bind only to their respective FRET cassettes to generate a target-specific signal.

CYP2D6-specific Triplex PCR for Genotyping

The CYP2D6 region encompasses approximately 5 kb of genomic sequence. While this is well within the capabilities of long-range PCR technologies, it depends heavily on template quality. With this in mind, to improve the robustness of the PCR reaction, the CYP2D6 genomic region was divided into three shorter and non-overlapping PCR fragments to pool together in a single triplex PCR reaction. All primers were designed over CYP2D6-specific sequence within 5′-, 3′- or intronic regions and to have a melting temperature of 68° C. (FIG. 124 contains the primer sequences used). Primer pair 1 amplifies exons 1 and 2 generating a 2036-bp product, primer pair 2 amplifies exons 3 to 6 generating a 1683-bp product and primer pair 3 armplifies exons 7 to 9 generating a 1754-bp product.

DNA from a group of 181 anonymous donors was used in this set of experiments. The DNA was isolated using the Qiagen QIAmp whole blood kit (Qiagen, Valencia, Calif.). The CYP2D6-specific triplex PCR reactions were performed using the ‘Herculase Hotstart’ PCR system (Stratagene, La Jolla, Calif. Cat. No.600310) with 10-200 ng of genomic DNA, 250 μM dNTPs, 0.4 μM of each primer, 2% DMSO and 2.5 units of the enzyme supplied in a final volume of 50 μL. The reaction was incubated on a ThermoHybaid PCR express Thermocycler (ThermoHybaid, Franklin, Mass.) using the following cycling parameters: 95° C. for 5 minutes, followed by 35 cycles of 95° C. for 30 seconds and 68° C. for 4 minutes, and finishing with a 10-minute extension cycle at 68° C. For verification purposes, 10 μl of the PCR product was initially visualised on a 1% agarose gel containing ethidium bromide. FIG. 123C provides an example of the three PCR products generated in this step.

INVADER CYP2D6 Genotyping Assays

INVADER assays were designed for the following CYP2D6 polymorphisms: CYP2D6*2-2850C to T; *2-4180G to C; *3-2549A Del; *4-1846G to A; *6-1707T Del; *10-100C to T; * 11-883G to C; *18-4125GTGCCCACT Duplication; *33-2483G to T; *35-31 G to A and *37-1943G to A. The number after the * represents the CYP2D6 haplotype and the number after the hyphen represents the position of the polymorphism in relation to the translational start codon (Daly, A. K., et al., Pharmacogenetics, 1996. 6(3): p. 193). FIG. 123B indicates the relative positions of these 11 assays in the CYP2D6 genomic region. Each assay was designed for non-synonymous polymorphisms. The CYP2D6*2, *10, *33 and *35 haplotypes are among the most common functional alleles in Caucasians aside from CYP2D6* 1, and CYP2D6*3; *4 and *6 are among the most common non-functional alleles in Caucasians apart from the deletion allele CYP2D6*5 (Gaedigk, A., et al., Pharnacogenetics, 1999. 9(6):669; Marez, D., et al, Pharmacogenetics, 1997. 7(3):193). Each PCR product is was detected by at least two INVADER assays. The Table in FIG. 124 provides the sequences for the INVADER and probe oligonucleotides for each assay. Each assay used a synthetic oligonucleotide complementary to both the INVADER and probe oligonucleotides as a positive control. The INVADER reactions were performed using 384-well INVADER Assay FRET detection plates, which contain CLEAVASE enzyme, F dye (F=fluorescein) and R dye (R=REDMOND RED) FRET cassettes, and reaction buffer, dried down in each well. REDMOND RED is from Synthetic Genetics, San Diego, Calif. Cassettes are shown in the Cassette Table, below.

Briefly, 3 μl of a 1/20 dilution of the CYP2D6-specific PCR products or a negative control (T10e0.1 buffer (10 mM Tris, pH 8, 0.1 mM EDTA)) were added to the appropriate wells followed by addition of 3 μl of the appropriate primary probes/INVADER oligonucleotide/MgCl₂ mix. After the additions, each reaction was overlayed with 7 μl of molecular biology-grade mineral oil to prevent evaporation (Sigma-Aldrich, Steinheim, Germany). Each 6-μl reaction contained 10 ng CLEAVASE enzyme, 4% PEG 8000, 2% glycerol, 0.06% NP 40, 0.06% Tween 20, 12-ug/ml BSA, 0.58 μM each of F dye and R dye FRET cassettes, 7 mM MgCl2, 0.7 lM of each allele-specific primary probe, and 0.07 μM INVADER oligonucleotide. Following reagent dispensing, plates were spun for 10 seconds at 1,000 rpm, then incubated at 95° C. for 5 minutes and then 63° C. for 30 minutes using ThermoHybaid PCR Express Thermocycler. Fluorescence was measured directly at the end of the incubation period using a CytoFluor 4000 fluorescence plate reader (Applied Biosystems, Foster City, Calif.). The settings used were 485/20 nm excitation/bandwidth and 530/25 nm emission/bandwidth for F dye detection and 560/20 nm excitation/bandwidth and 620/40 nm emission/bandwidth for R dye detection.

CYP2D6 INVADER Copy Number Assay

The INVADER system is directly quantitative and can be used to identify gene copy number by comparing the target gene signal (CYP2D6) with that of a reference gene that is known to be non-polymorphic for either duplication or deletion, such as the α-actin gene. Therefore, by using the relative ratios of the CYP2D6 and reference gene signals from each assay (similar to the way that ratios of the wild-type and variant signals are used to score a genotype, as described below), the deletion and duplication alleles of CYP2D6 can be identified and quantitated.

With the approval of the University of Wisconsin—Madison Institutional Review Board, the CYP2D6 copy number was assayed in 205 patients presenting for surgery at the University of Wisconsin Hospitals. Genomic DNA was isolated from whole blood using the PUREGENE DNA Isolation Kit (Gentra Systems, Minneapolis, Minn.) according to manufacturer's directions. INVADER detection of the CYP2D6 copy number was performed in duplicate using 96-well dry-down plates. In brief, 7 μl of pre-denatured DNA samples (15-20 ng/μl) or negative control (10 ng/μl solution of tRNA in T10e0.1 buffer (10 mM Tris, pH 8, 0.1 mM EDTA)) were added to the appropriate wells followed by addition of 8 μl of the appropriate primary probes/INVADER oligonucleotide/MgCl2 mix and then overlayed withl 5 μl of molecular biology grade mineral oil (Sigma-Aldrich, Steinheim, Germany). Each 15-μl reaction contained 100 ng CLEAVASE enzyme, 4% PEG 8000, 2% glycerol, 0.06% NP 40, 0.06% Tween 20, 12 ug/ml BSA, 0.35 μM of each F dye and R dye FRET cassettes, 7.5 mM MgCl2, 0.7 μM of each allele-specific primary probe, and 0.07 μM INVADER oligonucleotide. Following the reagent dispensing, plates were spun for 10 seconds at 1,000 rpm, incubated at 63° C. for 4 hours in a PTC 100 thermocycler (MJResearch, Incline Village, Nev.) and then directly read in a Cytofluor 4000 fluorescence plate reader (Applied Biosystems, Foster City, Calif.) using the same settings given above.

Assignments based on INVADER assay results were confirmed by long-range PCR. If CYP2D6 is deleted (CYP2D6*5), then a 3.5-kb PCR product will result (Steen, V. M., et al., Pharmacogenetics, 1995. 5(4):215). If there are duplicated or multiple copy CYP2D6 alleles then a 10-kb PCR product will result. Samples identified by the INVADER assay as either containing one or three copies of the CYP2D6 allele were subjected to PCR. Both the gene deletion and duplication PCR assays were performed with the GeneAmp XL PCR kit (Perkin Elmer, Foster City, Calif., Cat. No. N808-0192). The deletion primers (FIG. 22) were used in a 50-μl PCR reaction with 200 ng DNA, 1× XL reaction buffer, 1.1 mM Mg(OAc)₂, 200 mM of each dNTP, 0.3 mM of each primer, and 1 unit of DNA polymerase. The cycling parameters used were: 94° C. for 1 minute followed by 35 cycles of 94° C. for 1 minute, 65° C. for 30 seconds and 68° C. for 5 minutes, and then finishing with a 12-minute 72° C. extension cycle. The resulting 3.5-kb PCR products were detected by gel electrophoresis on a 1% agarose gel containing ethidium bromide. The duplication PCR primers amplified a fragment between exon 9 of the proximal CYP2D6 copy and intron 2 of a distal CYP2D6 copy in regions specific to CYP2D6 (Johansson, I.,et al., Pharmacogenetics, 1996. 6(4):351; FIG. 124). The 50-μl PCR reaction contained 400 ng DNA, 1× XL reaction buffer, 1.0 mM Mg(OAc)₂, 200 mM of each dNTP, 0.3 mM of each primer, and 3 units of DNA polymerase. The cycling parameters used were: 94° C. for 1 minute followed by 35 cycles of 94° C. for 1 minute, 61.4° C. for 30 seconds, and 68° C. for 16 minutes finishing with a 12-minute 72° C. extension cycle. The resulting 10-kb PCR products were detected by gel electrophoresis on a 1% agarose gel containing ethidium bromide. We observed no amplification in alleles lacking the duplication. However, conventional PCR could not determine the number of CYP2D6 duplications.

Data Analysis for Genotype and Copy Number Determination

Data were exported into the Microsoft Excel program (Microsoft, Redmond, Wash.). For each allele of a given polymorphism, the Net Fold Over Zero (FOZ−1) values are calculated as follows:

$$Net F dye FOZ = \frac{F dye raw counts from sample}{F dye raw counts from negative control} - 1$$

$$Net R dye FOZ = \frac{R dye raw counts from sample}{R dye raw counts from negative control} - 1$$

Determination of the genotype or copy number was based on the ratio of the Net R dye FOZ value to the Net F dye FOZ value as shown below:

$$Allelic Ratio = \frac{Net R dye FOZ}{Net F dye FOZ}$$

In cases where the Net FOZ value was equal to or less than zero, the Net FOZ value was adjusted to 0.01 to avoid the generation of negative values or division by zero. An allelic ratio of equal to or less than 0.25 was scored as homozygous for the F dye allele, a ratio greater than 4 was scored as homozygous for the R dye allele and a ratio greater than 0.25 but less than 4 was scored as heterozygous. Values that fell within two ranges (greater than 0.25 and equal to or less than 0.4, or equal to or greater than 2.5 and equal to or less than 4) were designated as equivocal and the sample result was not included. Instances in which both the F dye and R dye Net FOZ values were less than 2, were recorded as low-signal. The allelic ratio was calculated separately for each of the sample duplicates. If the two results were discordant, the sample result was not included.

For the copy number assay, the same Net R Dye FOZ and Net F dye FOZ (CYP2D6/a-actin) were calculated as above and the ratio of the a-actin to CYP2D6 NET FOZ was calculated to identify CYP2D6 copy number (R Dye NET FOZ/F Dye NET FOZ, as with the allelic ratio formula above). To identify gene copy number the following cutoffs were used: a ratio less than 0.35 was scored as a single CYP2D6 gene copy, a ratio equal to or greater than 0.40 but less than 0.60 as two gene copies and a ratio equal to or greater than 0.65 as three gene copies. Ratios that fell within the two ranges (equal to or greater than 0.35 but less than 0.40 and equal to or greater than 0.60 but less than 0.65 were scored as equivocal and the sample result was not included.

Of the 181 genomic DNA samples used for the CYP2D6 gene amplification assays, 171 were detected by standard agarose gel electrophoresis. Out of the 10 DNAs that did not generate a visible PCR product three could still be detected by INVADER assays and were included in the analysis. The remaining seven DNAs were considered degraded and not used. All INVADER reactions were performed in duplicate and each reaction was scored independently for genotype. A final genotyping score was recorded only if the results for the duplicates were concordant. From a possible 1,914 results for the 11 loci and 174 DNA samples, 1,904 unambiguous genotyping scores were recorded; only ten genotyping scores could not be assigned. Of these ten assays, four were invalid because of low signal in both duplicates and six were invalid because signal ratios from both duplicates fell within the equivocal ranges. All ten invalid assays were from the three DNA samples that did not generate visible triplex PCR products. These results are 99.5% concordant overall, and 100% concordant if only those samples that produced a PCR product detectable by ethidium bromide staining are included.

FIG. 125 contains four graphs as representative examples of the Net FOZ values (FOZ−1) from the 11 different assays; the resulting allele frequencies are presented in FIG. 126. All samples yielded heterozygous or homozygous variant signals except for CYP2D6*11-883, *18-4125 and *37-1943. These three alleles have a previously reported frequency of 0.1% or less (Marez, D., et al., Pharmacogenetics, 1997. 7(3):193); their absence in the 174 individuals analysed here is therefore not unexpected. The allele frequencies found in this study are comparable to published allele frequency values for Caucasians. The CYP2D6*2-2850 assay does not satisfy the Hardy-Weinberg equilibrium, however, Graph 1 in FIG. 125 shows a very clean separation of data. Further, all duplicates are in strong concordance and no unpredicted haplotypes were detected. With a relatively small sample size, a p value of 0.01 may be within acceptable limits.

Individual CYP2D6 haplotypes were constructed using the Clarke method (Clark A G., 1990, Mol Biol Evol 7:111-122) with the aid of information on the web site (http://www.imm.ki.se/CYPalleles/cyp2d6.html) and compound haplotypes were assigned to individuals. Allele and haplotype frequencies were also independently calculated using the expectation maximisation (EM) algorithm implemented in the Arlequin software (http://lgb.unige.ch/arlequin/) (Schneider, S., D. Roessli, and L. Excoffier, Arlequin ver. 2.000: A software for population genetics data analysis. 2000, genetics and Biometry Laboratories, University of Geneva; Slatkin, M. and L. Excoffier, Heredity, 1996. 76(Pt 4):377). The EM algorithm identified 9 different haplotypes within the 172 samples that yielded concordant genotyping information (FIG. 127). These haplotypes co-segregated into 22 different compound haplotypes, as inferred by the Clarke method (FIG. 128). Ten individuals carried two non-functional CYP2D6 alleles and 70 individuals carried a single functional allele (FIG. 128).

The copy number assay (205 samples) identified 17 single-copy individuals, 170 two-copy individuals and 17 three-copy individuals. The results from one assay fell into the equivocal range described above. As Graph 5 in FIG. 125 clearly illustrates, the groupings of one, two, and three copies of the CYP2D6 gene are distinctly separated. Sixteen of the 17 gene deletions detected by the INVADER assay were confirmed by long-PCR. Eleven of the 17 gene duplications detected by the INVADER assay were confirmed by long-PCR. Generating lengthy PCR products requires pure and intact genomic DNA. Any fragmentation of the DNA template will lead to failure of PCR. Therefore, the absence of a long PCR product cannot in and of itself confirm the absence of the CYP2D6 duplication or deletion.

The INVADER assays provided an unambiguous genotyping determination for 100% of the 171 samples that yielded a visible PCR product on an agarose gel. The overall unambiguous genotyping determination was 95.9%, but this lower success rate can largely be attributed to PCR amplification failure. This poor PCR amplification most likely arises from partial degradation of the genomic samples used, because more than 40% of the same 181 samples also failed to yield a 5-kb PCR product in initial CYP2D6 long-range PCR amplification attempts (data not shown). The triplex PCR approach described here will generate a CYP2D6-specific template from all but the most degraded DNA samples and may be more robust than protocols involving long-range PCR.

High failure rates inherent to long-PCR-based methods make the feasibility of using PCR-based methods to detect CYP2D6 copy number questionable. The accurate and automatable quantitative screening strategy we used to resolve CYP2D6 copy number alleles complements the PCR-INVADER genotyping assays well and avoids the problems associated with previous long-range PCR or RFLP methods.

In practise, this format is well suited to large-scale clinical trial or drug safety studies. It provides a rapid, comprehensive, high-throughput and ‘hands off’ method of achieving the high-resolution genotyping data needed to accurately predict CYP2D6 phenotypes. In addition, this preliminary study demonstrates the benefits of a clinical CYP2D6 genetic assay. Ten of the 174 DNA samples tested in the genotyping study possessed two non-functional alleles (FIG. 128) and 17 samples in the copy number study possessed a deleted allele. This information could be critical in a health care setting to avoid prescribing medications that are toxic at high doses. Equally, an individual homozygous for CYP2D6*35 may need higher doses of medication to achieve therapeutic levels due to the elevated enzyme activity observed in some *35 individuals. When prescribing medications to extensive metabolizers, health care providers must also consider whether potentially toxic metabolites would accumulate or whether a therapeutic level of medication would be reached. The quantitative nature of the INVADER assay is even more significant for extensive and ultra-rapid metabolizers. Complementing the PCR-INVADER genotyping assay with the genomic DNA copy number assay would be invaluable in identifying extensive and ultra-rapid metabolizers as well as the deleted alleles.

CASSETTE TABLE

| | | |
|---|---|---|
| *10 probe 2, *6 probe | FRET 6, | Y-tct-X-tcg-gcc-ttt-tgg- |
| 1, *4 probe 1, *3 probe | Red/Z28, | ccg-aga-gac-ctc-ggc-gcg-hex |
| 1, *2-2850 probe 2, | (931-74-10) | |
| *2-4180 probe 1, *18 | | |
| probe 1, *11 probe 1, | | |
| *35 probe 2, *33 probe | | |
| 2, *37 probe 2 | | |
| *10 probe 1, *6 probe | FRET 7, | Y-tct-X-agc-cgg-ttt-tcc- |
| 2, *4 probe 2, *3 probe | FAM/Z28, | ggc-tga-gag-tct-gcc-acg- |
| 2, *2-2850 probe 1, | (931-74-02) | tca-t-hex |
| *2-4180 probe 2, *18 | | |
| probe 2, *11 probe2, | | |
| *35 probe 1, *33 probe | | |
| 1, *37 probe 1 | | |

CYP 2D6 copy number

| ACTIN PRIMARY | FRET 16, FAM/Z28, | Y-tct-X-agc-cgg-ttt-tcc-ggc- |
|---|---|---|
| PROBE | (931-74-09) & | tga-gac-ctc-ggc-gcg-hex |
| | (1055-48-08) | |
| 2D6 PRIMARY | FRET 13, Red/Z28, | Y-tct-X-tcg-gcc-ttt-tgg-ccg- |
| PROBE | (1109-20-01) | aga-gac-tcc-gcg-tcc-gt-hex |

Y is FAM or RED
X is Z28
hex is hexane

B. Detection Assays and Drugs

Most prescription drugs are currently prescribed at standard doses in a “one size fits all” method. This “one size fits all” method, however, does not consider important genetic differences that give different individuals dramatically different abilities to metabolize and derive benefit from a particular drug. Genetic differences may be influenced by race or ethnicity (See FIG. 87). As such, certain groups of people considered at high risk (e.g. for an adverse drug reaction) are tested with a detection assay prior to administration of the drug. Also, detection assays (e.g. in panels) to identify which classes of patients will likely receive benefit from a candidate drug being developed.

If a health care provider knows both which genetic markers in particular DMEs are important for a particular drug and which variations of those genetic markers a patient has, it will be significantly easier to avoid dangerous ADRs. The genetic diagnostic panels of DME variations provided by the present invention allow one to determine the best course of treatment for each patient and to prescribe the most appropriate drug at the safest dosage, all based on an simple, easy-to-use assessment of the patient's unique genetic make-up.

Genetic markers for drug-metabolizing enzymes (DMEs) have enormous potential for dramatically altering the process that determines not only whether a drug enters the market, but also whether a drug that has been withdrawn can be “revitalized.” Individual responses to a particular drug often arise from variations within the genes that produce DMEs. An understanding of which DMEs are involved with helping the body eliminate a particular drug will be coupled with the knowledge of variations cause the body to metabolize the drug too quickly or too slowly. This important medical insights forms the foundation for high-resolution genetic diagnostic panels of thousands of DME variations that find use by health care providers before prescribing a particular drug. Those found to have genetic variation(s) associated with an adverse response to a particular drug are prescribed a different drug, one that is safe for them. Patient safety is enhanced significantly and those in desperate need of the therapeutic effects of a drug that has been withdrawn from the marketplace once again have access to an effective medication.

The development of a single new drug is estimated to cost $500 million, with much of the expense being incurred in the final phases. The use of DME markers of the present invention increases the efficiency of drug development in every phase, but is particularly useful in eliminating potentially toxic compounds from development in the earliest phases, before the majority of development dollars have been spent. Even after the expense of development, it is estimated that the most commonly used drugs will be effective in only 30-60 percent of patients with the same illness or disease. DME markers are used during drug development for the parallel development of genetic diagnostics that are administered at the point of care to avoid adverse drug reactions and improve the effectiveness of the drug. Thus, the present invention improves target discovery (the identification of new drug targets), preclinical toxicity determinations (the elimination of compounds that might cause ADRs early in the development process), lead compound prioritization (the prioritization of potential new drug compounds that have the desired effect and show no potential for ADRs), and clinical trial patient stratification (the ability to select potential participants with similar DMEs for clinical studies).

Representative drugs that have been withdrawn from the market since 1997 are shown in Table 9.

TABLE 9

| Withdrawn | Clinical Name | Reason for Using | ADR |
|---|---|---|---|
| 2001 | Cerivastatin | Cholesterol | Muscle cells |
| | | control | damage |
| 2001 | Repacuronium | Muscle relaxant | Breathing problems |
| | bromide | | |
| 2000 | Alosetron | Spastic colon | Liver damage |
| | hydrochloride | | |
| 2000 | Cisapride | Heartburn | Heartbeat problems |
| 2000 | Troglitizone | Type 2 diabetes | Liver damage |
| 1999 | Astemizole | Allergies | Heart problems |
| 1998 | Bromfenac | Pain relief | Liver damage |
| 1998 | Mibefradil | High blood | Drug interactions |
| | | pressure | |
| 1997 | Fenfluramine and | Obesity | Heart valve damage |
| 1997 | Phentermine | Obesity | Heart valve damage |

C. Screening Methods for Selecting Drug Therapy

As described above, nucleic acid detection assays may be employed to screen subjects in order to facilitate drug therapy and avoid problems of toxicity or lack of efficacy. In this regard, subjects may be screened with a nucleic acid detection assay (e.g. as described above) prior to the administration of a drug. The results of the detection assay may indicate that the subject does not have a polymorphism that has been shown to lead to negative consequences upon administration of the drug (e.g. toxicity, or lack of efficacy). In this situation, the subject may be administered the drug. In other embodiments, the results of the detection assay indicate that the subject has a polymorphism linked to an adverse reaction to the drug. In this situation, the subject is not administered the drug or administered a different dose of the drug. Alternatively, the subject may still be administered the drug along with a second drug that counters the negative effect of the first drug (e.g. reducing side effects, or making the first drug effective).

In preferred embodiments, the nucleic acid detection assay is on a panel capable of detecting at least two polymorphisms. In some embodiments, the polymorphisms on the panel all relate to the ability of a subject to safely or effectively utilize a certain drug (e.g. the panel comprises at least two nucleic acid detection assays configured to determine if a subject has a polymorphisms in a particular drug metabolizing enzyme).

In some embodiments, a subject may be screened with a nucleic acid detection assay, and then given a drug based on the results of the assay. However, even if the drug is effective in the patient and does not cause severe toxicity, the drug may cause uni-wanted side effects. Therefore, the subject may then be screened for ability to utilize a second drug to counteract the side effects of the first drug. In this manner, the information on polymorphism affecting the second drug may be generated and collected (thereby allowing a health care professional to know if a second drug should be given to counteract the effect of a first drug).

In certain embodiments, the drug and a nucleic acid detection assay useful in determining if a subject should receive (or continue to receive) a drug are marketed and/or sold together. In this regard, the proper detection assay is available to a physician or other users such that an informed decision to administer a drug to a particular patient may be made. In preferred embodiments, the results of testing a subject for a polymorphism is stored in a computer database. This database may be accessed by doctors, pharmacists, or other user to determine the correct prescription for the subject. For example,-the subject may have a disease that requires a certain type of drug. The computer database may be queried for this subject to determine if this drug would be safe and/or effective for the patient, or if the subject should be administered a different drug, or a second drug to reduce problems with the first drug.

In other embodiments, the multiplex PCR methods described above (See, section II. E. entitled “Multiplex PCR Primer design”) may be employed to design multiplex PCR reactions that amplify multiple target sequences, and allow for a detection assay to be performed (e.g. without interference with the primers). In this regard, multiple alleles that are known, or believed to cause safety or efficacy concerns in a subject may be analyzed simultaneously to determine if the subject should be administered a certain drug. This is important as any one polymorphism may indicate that the patient should not be given the drug, or be given a different dosage, or given a second drug to counteract the effects of the second drug. Such multiplex reactions also allow additional targets to be amplified and detected that relate to the ability of a second drug to safely and effectively counter act any negative affects of a first drug.

In some embodiments, the present invention provides methods for extending the patent protection of a patented pharmaceutical. For example, while a pharmaceutical that is patented may eventually go off patent, the combination of screening for a certain polymorphism prior (or during) administration of a drug may be patented, thus providing additional patent protection. Thus, the present invention provides methods whereby a useful detection assay is associated with a patented drug, and patents are drafted and applied for based on the assay-drug combination.

As described above, nucleic, acid detection assays may be employed to screen subjects in order to facilitate drug therapy and avoid problems of toxicity or lack of efficacy. In this regard, subjects may be screened with a nucleic acid detection assay (e.g. as described above) prior to the administration or a drug. The results of the detection assay may indicate that the subject does not have a polymorphism that has been shown to lead to negative consequences upon administration of the drug (e.g. toxicity, or lack of efficacy). In this situation, the subject may be administered the drug. In other embodiments, the results of the detection assay indicate that the subject has a polymorphism linked to an adverse reaction to the drug. In this situation, the subject is not administered the drug or administered a different dose of the drug. Alternatively, the subject may still be administered the drug along with a second drug that counters the negative effect of the first drug (e.g. reducing side effects, or making the first drug effective). In this application, Table 11 is Table 1 from U.S. application Ser. No. 10/035,833 filed Dec. 27, 2001 and which is expressly incorporated by reference in its entirity.

In preferred embodiments, the nucleic acid detection assay is on a panel capable of detecting at least two polymorphisms. In some embodiments, the polymorphisms on the panel all relate to the ability of a subject to safely or effectively utilize a certain drug (e.g. the panel comprises at least two nucleic acid detection assays configured to determine if a subject has a polymorphisms in a particular drug metabolizing enzyme).

In some embodiments, a subject may be screened with a nucleic acid detection assay, and then given a drug based on the results of the assay. However, even if the drug is effective in the patient and does not cause severe toxicity, the drug may cause un-wanted side effects. Therefore, the subject may then be screened for ability to utilize a second drug to counteract the side effects of the first drug. In this manner, the information on polymorphism affecting the second drug may be generated and collected (thereby allowing a health care professional to know if a second drug should be given to counteract the effect of a first drug).

In certain embodiments, the drug and a nucleic acid detection assay useful in determining if a subject should receive (or continue to receive) a drug are marketed and/or sold together. In this regard, the proper detection assay is available to a physician or other users such that an informed decision to administer a drug to a particular patient may be made. In preferred embodiments, the results of testing a subject for a polymorphism is stored in a computer database. This database may be accessed by doctors, pharmacists, or other user to determine the correct prescription for the subject. For example, the subject may have a disease that requires a certain type of drug. The computer database may be queried for this subject to determine if this drug would be safe and/or effective for the patient, or if the subject should be administered a different drug, or a second drug to reduce problems with the first drug.

In other embodiments, the multiplex PCR methods described above (See, section II. E. entitled “Multiplex PCR Primer design”) may be employed to design multiplex PCR reactions that amplify multiple target sequences, and allow for a detection assay to be performed (e.g. -without interference with the primers). In this regard, multiple alleles that are known, or believed to cause safety or efficacy concerns in a subject may be analyzed simultaneously to determine if the subject should be administered a certain drug. This is important as any one polymorphism may indicate that the patient should not be given the drug, or be given a different dosage, or given a second drug to counteract the effects of the second drug. Such multiplex reactions also allow additional targets to be amplified and detected that relate to the ability of a second drug to safely and effectively counter act any negative affects of a first drug.

In some embodiments, the present invention provides methods for extending the patent protection of a patented pharmaceutical. For example, while a pharmaceutical that is patented may eventually go off patent, the combination of screening for a certain polymorphism prior (or during) administration of a drug may be patented, thus providing additional patent protection. Thus, the present invention provides methods whereby a useful detection assay is associated with a patented drug, and patents are drafted and applied for based on the assay-drug combination.

In some embodiments, the genes and nucleic acid sequences containing polymorphisms are found in publications such as WO0050639, WO0004194, WO0153460, and U.S. Application Publication Number 20010034023A1, all of which are hereby incorporated by reference for all purposes. These applications also, for example, provide methods for identifying disease causing polymorphisms and selecting drug therapy (See, e.g., Examples 6-9 of WO0050639, hereby specifically incorporated by reference). Also useful in this regard is FIG. 131. FIG. 131 shows a table useful in correlating particular genotypes with particular phenotypes, and further correlating particular drugs with particular diseases. In particular, this figure shows many genes and the pathways and/or function for these genes. This figure also shows various diseases and the pathways typically associated with these diseases (allowing one to refer back to genes that in this figure that may then be involved with these diseases). This figure further shows many polymorphisms that are present in certain genes (thereby allowing one to identify polymorphisms associated with a gene that is associated with a disease). Finally, this figure provides a list of therapeutic agents and the action and/or disease the therapeutic agent is used for. In this regard, one employing this figure to identify polymorphisms that could be tested, for example, for in a patient with a particular disease prior to administering a particular therapeutic agent to the patient. This figure is also useful in combination with Tables 8, 9, 10, 11, 12 and FIG. 96 in order to personalize drug therapy for a patient.

In certain embodiments, the present invention provides methods method for selecting a treatment for a patient suffering from a disease, disorder, or condition comprising: determining whether cells of the patient contain at least one polymorphism in a gene or nucleic acid sequence present in Tables 8, 9, 10, 11, 12, or FIG. 96, wherein the presence or the absence of the at least one polymorphism in the gene or the nucleic acid sequence is indicative of the effectiveness of the treatment for the disease, disorder, or condition. In some embodiments, the at least one polymorphism comprises a plurality of polymorphisms. In particular embodiments, the plurality of polymorphisms comprises: i) at least one polymorphism shown in Tables 8, 9, 10, 11, 12, or FIG. 96, and ii) at least polymorphism shown in FIG. 131. In some embodiments, the disease, disorder, or condition is listed in FIG. 131 or Table 9.

In certain embodiments, the presence of the at least one polymorphism is indicative that the treatment will be effective for the patient. In other embodiments, the presence of the polymorphism is indicative that the treatment will be ineffective or contra-ihdicated for the patient. In some embodiments, the plurality of polymorphisms comprise a haplotype or haplotypes. In additional embodiments, the selecting a treatment further comprises identifying a compound differentially active in a patient bearing a form of the gene or the nucleic acid sequence containing the at least one polymorphism. In certain embodiments, the compound is a compound listed in Table 9 or FIG. 131.

In some embodiments, the selecting a treatment further comprises excluding or eliminating a treatment, wherein the presence or absence of the at least one polymorphism is indicative that the treatment will be ineffective or contra-indicated. In further embodiments, the treatment comprises a first treatment and a second treatment, the method comprising the steps of identifying the first treatment as effective to treat the disease, disorder, or condition; and identifying a the second treatment which reduces a deleterious effect or promotes efficacy of the first treatment. In other embodiments, the selecting a treatment further comprises selecting a method of administration of a compound effective to treat the disease in a patient, disorder or condition, wherein the presence or absence of the at least one polymorphism is indicative of the appropriate method of administration for the compound. In some embodiments, the selecting the method of administration comprises selecting a suitable dosage level or frequency of administration of a compound. In additional embodiments, the methods further comprise determining the level of expression of the gene or nucleic acid sequence, or the level of activity of a protein containing a polypeptide expressed from the gene or nucleic acid sequence, wherein the combination of the determination of the presence or absence of the at least one polymorphism and the determination of the level of activity or the level of expression provides a further indication of the effectiveness of the treatment.

In particular embodiments, the methods further comprise determining at least one of: sex, age, racial origin, ethnic origin, and geographic origin of the patient, wherein the combination of the determination of the presence or absence of the at least one polymorphism and the determination of the sex, age, racial origin, ethnic origin, and geographic origin of the patient provides a further indication of the effectiveness of the treatment. In other embodiments, the disease, disorder, or condition is selected from the group consisting of neoplastic disorders, amyotrophic lateral sclerosis, anxiety, dementia, depression, epilepsy, Huntington's disease, migraine, demyelinating disease, multiple sclerosis, pain, Parkinson's disease, schizophrenia, spasticity, psychoses, and stroke, drug-induced diseases, disorders, or toxicities consisting of blood dyscrasias, cutaneous toxicities, systemic toxicities, central nervous system toxicities, hepatic toxicities, cardiovascular toxicities, pulmonary toxicities, and renal toxicities, arthritis, chronic obstructive pulmonary disease, autoimmune disease, transplantation, pain associated with inflammation, psoriasis, arteriosclerosis, asthma, inflammatory bowel disease, and hepatitis, diabetes mellitus, metabolic syndrome X, diabetes insipidus, obesity, contraception, infertility, hormonal insufficiency related to aging, osteoporosis, acne, alopecia, adrenal dysfunction, thyroid dysfunction, and parathyroid dysfunction, anemia, angina, arrhythmia, hypertension, hypothennia, ischemia, heart failure, thrombosis, renal disease, restenosis, and peripheral vascular disease.

In some embodiments, the detection of the presence or absence of the at least one polymorphism comprises amplifying a segment of nucleic acid including at least one of the polymorphisms. In further embodiments, the detection of the presence or absence of the at least one polymorphism comprises multiplex amplification of a plurality of segments of nucleic acid each including at least one of the polymorphisms. In certain embodiments, the segment of nucleic acid is 500 nucleotides or less in length, 100 nucleotides orless in length, or 45 nucleotides or less in length. In other embodiments, the segment includes a plurality of polymorphisms. In additional embodiments, the amplification preferentially occurs from one of the two strands of a chromosome.

In certain embodiments, the determining comprises employing a detection assay selected from a TAQMAN-assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In other embodiments, the detection of the presence or absence of the at least one polymorphism comprises sequencing at least one nucleic acid sequence. In, some embodiments, the detection of the presence or absence of the at least one polymorphism comprises mass spectrometric determination of at least one nucleic acid sequence. In further embodiments, the detection of the presence or absence of the at least one polymorphism comprises determining the haplotype of a plurality of polymorphisms in a gene. In preferred embodiments, the determining comprises employing a detection assay, wherein the detection assay employs a structure specific nuclease (e.g. an INVADER assay or TAQMAN assay).

In some embodiments, the present invention provides methods for selecting a treatment for a patient suffering from a disease, disorder, or condition comprising: determining whether cells of the patient contains: i) a first polymorphism present in a gene or nucleic acid sequence in Tables 8, 9, 10, 11, 12, or FIG. 96, and ii) a second polymorphism present in a gene or nucleic acid sequence in FIG. 131, wherein the presence or the absence of the first and second polymorphisms is indicative of the effectiveness of the treatment for the disease, disorder, or condition. In other embodiments, the present invention provides methods for selecting a treatment for a patient suffering from a disease, disorder, or condition comprising: determining with a detection assay employing a structure specific nuclease whether cells of the patient contain at least one polymorphism in a gene or nucleic acid sequence present in Tables 8, 9, 10, 11, 12, FIG. 96, FIG. 131, wherein the presence or the absence of the at least one polymorphism in the gene or the nucleic acid sequence is indicative of the effectiveness of the treatment for the disease, disorder, or condition.

In other embodiments, the present invention provides pharmaceutical compositions comprising a compound which has a differential effect in patients having at least one copy of a particular form of an identified gene or nucleic acid sequence from Tables 8, 9, 10, 11, 12, or FIG. 96; and a pharmaceutically acceptable carrier or excipient or diluent, wherein the composition is preferentially effective to treat a patient with cells comprising a form of the gene comprising at least one polymorphism. In some embodiments, the present invention provides pharmaceutical compositions comprising a compound which has a differential effect in patients having: i) at least one copy of a particular form of an identified gene or nucleic acid sequence from Tables 8, 9, 10, 11, 12, or FIG. 96, and ii) at least one copy of a particular form of an identified gene or nucleic acid sequence from FIG. 131.

In additional embodiments, the present invention provides nucleic acid probes comprising a nucleic acid sequence 7 to 200 nucleotide bases in length that specifically binds (e.g. under medium to high stringency conditions) to a nucleic acid sequence comprising at least one polymorphism in a gene from Tables 8, 9, 10, 11, 12, or FIG. 96, or a sequence complementary thereto or an RNA equivalent.

In some embodiments, the present invention provides methods for determining whether a compound has differential effects on cells containing at least one different form of a gene or nucleic acid sequence from Tables 8, 9, 10, 11, 12, or FIG. 96, comprising: contacting a first cell and a second cell with the compound, wherein the first cell and the second~ cell differ in the presence or absence of at least one polymorphism in the gene; and determining whether the responses of the first cell and the second cell to the compound differ, wherein the difference in the response is due to the presence or absence of the at least one polymorphism. In other embodiments, the present invention provides methods for determining whether a compound has differential effects on cells containing at least two different forms of a gene or nucleic acid sequence from Tables 8, 9, 10, 11, 12, FIG. 96, or FIG. 131, comprising: contacting a first cell and a second cell with the compound, wherein the first cell and the second cell differ in the presence or absence of at least two polymorphism in the gene, wherein at least one polymorphism is from Tables 8, 9, 10, 11, 12, and FIG. 96, and at least one polymorphism is from FIG. 131; and determining whether the responses of the first cell and the second cell to the compound differ, wherein the difference in the response is due to the presence or absence of the at least two polymorphisms.

In other embodiments, the present invention provides methods of treating a patient suffering from a disease or condition, comprising: a) determining whether cells of the patient contain a form of a gene from Tables 8, 9, 10, 11, 12, or FIG. 96 which comprises at least one polymorphism, wherein the presence or absence of the at least one polymorphism is indicative that a treatment will be effective in the patient; and b) administering the treatment to the patient. In certain embodiments, the determining employs a detection assay, and the detection assay employs a structure specific nuclease. In some embodiments, the present invention provides methods of treating a patient suffering from a disease or condition, comprising: a) determining whether cells of the patient contain: i) a form of a gene from Tables 8, 9, 10, 11, 12, or FIG. 96 which comprises a first polymorphism, and ii) a form of a gene from FIG. 131 which comprises a second polymorphism, wherein the presence or absence of the first and second polymorphisms is indicative that a treatment will be effective in the patient; and b) administering the treatment to the patient. In certain embodiments, the determining employs a detection assay, and the detection assay employs a structure specific nuclease.

In additional embodiments, the present invention provides methods of treating a patient suffering from a disease or condition, comprising: a) comparing the presence or absence of at least one polymorphism in a gene or nucleic acid sequence from Tables 8, 9, 10, 11, 12, or FIG. 96 in cells of a patient suffering from the disease or condition with a list of polymorphisms in the gene indicative of the effectiveness of at least one method of treatment; b) eliminating a method of treatment from the at least one method of treatment, wherein the presence or absence of at least one of the at least one polymorphism is indicative that the method of treatment will be ineffective or contra-indicated in the patient; c) selecting an alternative method of treatment effective to treat the disease or condition; and d) administering the alternative method of treatment to the patient. In some embodiments, the present invention provides methods of treating a patient suffering from a disease or condition, comprising: a) comparing the presence or absence of a first polymorphism in a gene or nucleic acid sequence from Tables 8, 9, 10, 11, 12, or FIG. 96 in cells of a patient suffering from the disease or condition with a list of polymorphisms in the gene indicative of the effectiveness of at least one method of treatment; b) comparing the presence or absence of a second polymorphism in a gene or nucleic acid sequence from FIG. 131; c) eliminating a method of treatment from the at least one method of treatment, wherein the presence or absence of the first and second polymorphisms is indicative that the method of treatment will be ineffective or contra- indicated in the patient; d) selecting an alternative method of treatment effective to treat the disease or condition; and e) administering the alternative method of treatment to the patient.

In other embodiments, the present invention provides methods for determining whether a polymorphism in a gene or nucleic acid sequence from Tables 8, 10, 11, 12, or FIG. 96 provides variable patient response to a method of treatment for a disease or condition, comprising: determining whether the response of a first patient or set of patients suffering from a disease or condition differs from the response of a second patient or set of patients suffering from the disease or condition; determining whether the presence or absence of at least one polymorphism in the gene differs between the first patient or set of patient and the second patient or set of patients; wherein correlation of the presence or absence of at least one polymorphism and the response of the patient to the treatment is indicative that the at least one polymorphism provides variable patient response. In certain embodiments, the present invention provides methods for determining whether a first polymorphism from Tables 8, 10, 11, 12, or FIG. 96, and a second polymorphism from FIG. 131 provides variable patient response to a method of treatment for a disease or condition, comprising: determining whether the response of a first patient or set of patients suffering from a disease or condition differs from the response of a second patient or set of patients suffering from the disease or condition; determining whether the presence or absence of the first and second polymorphisms differs between the first patient or set of patient and the second patient or set of patients; wherein correlation of the presence or absence of at least one polymorphism and the response of the patient to the treatment is indicative that the at least one polymorphism provides variable patient response.

In some embodiments, the present invention provides methods for determining a method of treatment effective to treat a disease or condition in a sub-population of patients, comprising altering the level of activity of a product of an allele of a gene or nucleic acid sequence from Tables 8, 9, 10, 11, 12, and FIG. 96; and determining whether the alteration provides a differential effect related to reducing or alleviating a disease or condition as compared to at least one alternative allele, wherein the presence of a the differential effect is indicative that the altering the level of activity comprises an effective treatment for the disease or condition in the sub-population.

In certain embodiments, the present invention provides methods for performing a clinical trial or study, comprising selecting or stratifying subjects using a polymorphism or polymorphisms or haplotypes from one or more genes specified in Tables 8, 10, 11, 12, or FIG. 96. In other embodiments, the methods further comprise selecting an additional polymorphism from FIG. 131. In further embodiments, the differential efficacy, tolerance, or safety of a treatment in a subset of patients who have a particular polymorphism, polymorphisms, or haplotype in a gene or genes, or nucleic acid sequence from Tables 8, 10, 11, 12, or FIG. 96 is determined, comprising; conducting a clinical trial and using a statistical test to assess whether a relationship exists between efficacy, tolerance, or safety with the presence or absence of any of the polymorphisms or haplotype in one or more of the genes, wherein results of the clinical trial or study are indicative whether a higher or lower efficacy, tolerance, or safety of the treatment in the subset of patients is associated with any of the polymorphism or polymorphisms or haplotype in one or more of the gene. In particular embodiments, the normal subjects or patients are prospectively stratified by genotype in different genotype-defined groups, including the use of genotype as a enrollment criterion, using a polymorphism, polymorphisms or haplotypes from Tables 8, 9, 10, 11, 12, and FIG. 96, and subsequently a biological or clinical response variable is compared between the different genotype- defined groups. In further embodiments, the normal subjects or patients in a clinical trial or study are stratified by a biological or clinical response variable in different biologically or clinically-defined groups, and subsequently the frequency of a polymorphism, polymorphisms or haplotypes from Tables 8, 9, 10, 11, 12, and FIG. 96 are measured in the different biologically or clinically defined groups. In some embodiments, the normal subjects or patients in a clinical trial or study are stratified by at least one demographic characteristic selected from the groups consisting of sex, age, racial origin, ethnic origin, or geographic origin.

In some embodiments, the present invention provides methods for identifying a patient for participation in a clinical trial of a therapy for the treatment of a disease or disorder, comprising identifying a patient with a disease risk and determining the patient's allele status for an identified gene or nucleic acid sequence from Tables 8, 10, 11, 12, and FIG. 96. In preferred embodiments, the allele status is determined with a detection assay, wherein the detection assay employs a structure specific nuclease. In certain embodiments, the present invention provides methods for identifying a patient for participation in a clinical trial of a therapy for the treatment of a disease or disorder, comprising identifying a patient with a disease risk and determining the patient's allele status for an identified gene or nucleic acid sequence from Tables 8, 9, 10, 11, 12, and FIG. 96, and determining the patient's allele status for a gene or nucleic acid sequence form FIG. 131. In preferred embodiments, the allele status is determined with a detection assay, wherein the detection assay employs a structure specific nuclease.

In certain embodiments, the present invention provides methods for treating a patient at risk for a disease, comprising identifying a patient with a risk for the disease; determining the allele status of the patient for at least one gene from Tables 8, 9, 10, 11, 12, and FIG. 96; and converting the genotypic allele status into a treatment protocol that comprises a comparison of the genotypic allele status determination with the allele frequency of a control population, thereby allowing a statistical calculation of the patient's risk for having the disease. In preferred embodiments, the allele status is determined with a detection assay, wherein the detection assay employs a structure specific nuclease. In additional embodiments, the methods further comprise determining the allele status of the patient for a gene or nucleic acid sequence from FIG. 131.

In some embodiments, the present invention provides methods for improving the safety of candidate therapies associated with having a disease, comprising comparing the relative safety of the candidate therapeutic intervention in patients having different alleles in one or more than one of the genes listed in Tables 8, 10, 11, 12, and FIG. 96, thereby identifying subsets of patients with differing safety of the candidate therapeutic intervention.

i. Irinotecan

An important, and currently available antineoplastic treatment, is called Irinotecan. Irinotecan's chemical formula name is (S)-4,11-diethyl-3,4,12,14-tetrahydro-4-hydroxy-3,14-dioxyo-1H-pyranol[3′,4′:6,7]-indolizino[1,2-b]quinolin-9-y[1,4′-bipeperidine]-1′-carboxylate, monohydrochloride, trihydrate. The empirical formula for Irinotecan is C₃₃H₃₈N₄O₆HCl3H₂O and has a molecular weight of 677.19. Irinotecan is currently sold under the name CAMPTOSAR by Pharmacia & Upjohn Corporation. Irinotecan is used to treat cancer (e.g., CAMPTOSAR is approved for colorectal cancer un the United States). The mechanism of action of Irinotecan and its active metabolize SN-38 is preventing topoisomerase I from functioning properly.

Irinotecan (also known as CPT-11) is transformed in vivo by carboxylesterases to an active metabolite called SN-38. SN-38 has about 100-1,000 fold higher antitumor activity than Irinotecan. Irinotecan has been shown to be metabolized by hepatic cytochrome P-450 3A enzymes to a compound called APC, which has a 500 fold weaker antitumor activity compared with SN-38. SN-38 is known to undergo significant bilary excretion and enterohepatic circulation. SN-38 is also subjected to glucuronidation by hepatic uridine diphosphate glucuronosyltransferases (UGTs) to form SN-38G. SN-38G is inactive and is excreted into the urine and bile. Failure to convert SN-38 to SN-38G has been suggested as a cause of diarrehea in patients administered Irinotecan due to an accumulation of SN-38 (See, Lyer et al., J. Clin. Invest., 101 (4), February, 1998, 847-854, herein incorporated by reference).

Clinical studies have shown that Irinotecan was able to significantly improve tumor response rates, time to tumor progression and survival. Irinotecan has shown effectiveness when administered with 5-fluorouracil (5-FU) and leucovorin (LV). Irinotecan is generally administered intravenously.

There are many side effects associated with Irinotecan therapy. One side effect is cholinergic symptoms (e.g. early-onset diarrhea, contraction of pupils, lacrimation, flushing, rhinitis, increased salivation, diaphoresis, and abdominal cramping). Administration of atropine is generally recommended to counteract these symptoms. Another known side effect is late-onset diarrhea, which may be treated with loperamide, IV hydration, and oral antibiotics). Another known side effect is nausea and vomiting. Administration of antiemetic agents on the day of Irinotecan treatment may be used to counteract nausea and vomiting. Finally, another, Irinotecan side effect is severe myelosuppression, with deaths due to sepsis being reported.

ii. UGTs, Irinotecan, and Nucleic Acid Screening

UGTs are microsdmal enzymes catalyzing the glucuronidation of numerous endogenous and exogenou's substrates. Glucuronidation increases the polarity of the substrates to allow them to be-better eliminated from the body. The human UGTs are classified into UGT1 and UGT2 families. The UGT1 gene consists of at least 13 unique isoforms with variable exon 1 and common exons 2 to 5. Each of the exons 1 is preceded by its own promoter and differentially spliced to the common exons to produce a unique mature mRNA. The UGT1 family is further classified into multiple isoforms, i.e., UGT1A1, UGT1A3, UGT1A4, up to UGT1A12. The UGT1A1 isoform is responsible for the glucuronidation of bilirubin. The clinically relevant polymorphisms related to genetic abnormalities in UGT1A1 are those associated with familial hyperbilirubinemic syndromes such as Crigler-Najjar syndromes type I (CN-I) and type II (CN-II), and Gilbert's syndrome. CN-I syndrome is rare and is associated with severe unconjugated hyperbilirubinemia. Patients with CN syndromes have absent (CN-I) or reduced (CN-II) UGT1A1 activity with corresponding unconjugated serum bilirubin levels of 15 to >50 mg/dl and 10 to 20 mg/dl, respectively. Gilbert's syndrome is a mild chronic unconjugated hyperbilirubinemia, with serum bilirubin levels usually <3 mg/dl, although higher, lower, and even normal values are not uncommon. A wide variation in the incidences of Gilbert's syndrome has been reported, ranging from 0.5 to 19% in various groups. Gilbert's syndrome is usually associated with homozygosity for the sequence (TA)₇TAA instead of (TA)₆TAA in the promoter region of the UGT1A1 gene, resulting in reduced UGT1A1 expression levels and activity.

In addition to (TA)₆ and (TA)₇ alleles, two new alleles with five and eight TA repeats, i.e., (TA)₅ and (TA)₈, have been found (See, Beutler et al., Proc Natl Acad Sci USA, 95:8170-8174, 1998; and DiRienzo et al., Clin Pharmacol Ther 63: 207, 1998, both of which are herin incorporated by reference). These alleles were present in population samples from African ancestry, where they occur at lower frequencies compared with the alleles (TA)₆ and (TA)₇. However, the first Caucasian subject affected by Gilbert's syndrome found to be heterozygous for the (TA)₈ allele has been recently described (See, lolascon et al., Haematologica 84: 106-109, 1999, herein incorporated by reference). Four alleles of the UGT1A1 promoter have been found in 379 individuals sampled at random from 11 aboriginal and admixed populations from different ethnic backgrounds. Allele frequencies vary considerably across ethnic groups, with Asian and American indian populations showing highest frequencies of allele (TA)₆. The frequency of allele (TA)₇ differs significantly between sub-Saharan Africans and Caucasians (See, Hall et al., Pharmacogenetics 9: 591-599, 1999, herein incorporated by reference).

There have been recent reports of heterozygous and homozygous missense mutations in the coding region of UGT1A1 in certain subjects with Gilbert's syndrome who do not have homozygous mutations at the promoter level. The Gly71Arg mutation in the coding region has been shown to result in a 30% (heterozygotes) and 60% (homozygotes) decrease in bilirubin glucuronidating activity.

UGT1A1 polymorphism plays several roles in the metabolism of irinotecan. The example of irinotecan demonstrates how a polymorphism in an inactivating metabolic pathway may affect the therapeutic outcome in cancer chemotherapy. As described above, Irinotecan (CPT-11; 7-ethyl-10-[4-(1-piperidino)-1-piperidino]carbonyloxycamptothecin) is a camptothecin derivative used in the treatment of metastatic colorectal cancer. Irinotecan is a prodrug, since it needs to be activated by systemic carboxylesterases to SN-38 (7-ethyl-10-hydroxycamptothecin) in order to exert its antitumor activity mediated by the inhibition of topoisomerase I. SN-38 undergoes glucuronide conjugation to form the inactive SN-38 glucuronide (SN-38G; 10-O-glucuronyl-SN-38). In addition, two oxidated metabolites of irinotecan have been identified as APC (7-ethyl-10[4-N-(5-aminopentanoic acid)-1 piperidino] carbonyloxycamptothecin) and NPC [7-ethyl-10-(4-amino-1-piperidino) carbonyloxycamptothecin] formed by CYP3A4 enzyme. APC and NPC have shown weak antitumor activity in vitro.

SN-38 has been associated with the severe diarrheal episodes occurring after irinotecan therapy as a result of the direct enteric injury caused by SN-38. Because it undergoes significant biliary excretion, SN-38 may potentially continue to remain in the gastrointestinal tract, resulting in prolonged diarrhea. The glucuronidation of SN-38 to the inactive SN-38G may protect against irinotecan-induced intestinal toxicities as a result of renal elimination of the more polar SN-38G.

The assessment of pharmacodynamics of SN-38 glucuronidation showed that, with respect to the total irinotecan available in the circulation, patients with relatively low glucuronidation rates had progressive accumulation of SN-38 leading to toxicity (Gupta et al., Cancer Res 54: 3723-3725, 1994). A genetic predisposition to the metabolism of irinotecan may be critical in patients with reduced UGT1A1 activity (Iyer et al., J Clin Invest 101:847-854, 1998, herein incorporated by reference). As the distinction between mild instances of the syndrome and normal condition is sometimes difficult, Gilbert's syndrome remains often undiagnosed.

Genotyping of UGT1A1 promoter mutations may predict the functional activity of UGT1A1. A correlation analysis with the corresponding phenotyping results is necessary to demonstrate the validity of the genotyping procedure. Iyer et al., (Clin Pharmacol Ther 65: 576-582, 1999, herein incorporated by reference) recently showed a good concordance between UGT1A1 promoter genotype and in vitro glucuronidation of SN-38 in human livers of Caucasian origin. SN-38 glucuronidation rates were significantly lower in homozygotes (TA)7/(TA)7 and heterozygotes (TA)6/(TA)7 when compared with the wild-type genotype (TA)6/(TA)6.

A high variability in SN-38 glucuronidation reported in liver samples from populations of African descent (Iyer et al., Clin Pharmacol Ther 65: 197, 1999, herein incorporated by reference) can be explained by the presence of five and eight TA repeats, i.e., (TA)5 and (TA)8, in the UGT1A1 promoter (see, Beutler et al., 1998; and DiRienzo et al., 1998). According to this evidence, greater and lesser glucuronidating activity of SN-38 has been found in (TA)5 and (TA)8 liver samples, respectively (see, Iyer et al., 1999). UGT1A1 activity is inversely related to the number of TA repeats, since the transcriptional activity of the promoter decreases with the progressive increase in the number of TA repeats (see, Beutler et al., 1998). These new alleles indicate that up to 10 genotypes may exist at the TATAA element, probably resulting in different phenotypes with regard to bilirubin conjugation and irinotecan pharmacokinetics. Based upon in vitro phenotyping of UGT1A1 activity in livers, homozygotes for (TA)7 and heterozygotes (TA)6/(TA)7 might be expected to have at least a 50 and 25% decrease in SN-38 glucuronidating activity, respectively (see, Iyer et al., 1999). A significantly impaired ability to glucuronidate SN-38 has been found in one patient genotyped as (TA)7 homozygote (Ando et al., Ann Oncol 9: 845-847, 1998, herein incorporated by reference). Consequently, appropriate irinotecan dose reductions may be necessary in homozygotes for (TA)7 and heterozygotes (TA)6/(TA)7.

As mentioned above, Irinotecan is known to metabolized by UGT's. As such, the present invention provides systems and methods for screening subjects that are candidates for Irinotecan administration, or patients already taking Irinotecan. Any type of detection assay may be employed including, but not limited to; a hybridization assay, a TAQMAN assay, or an invasive cleavage assay (e.g. INVADER assay), a mass spectroscopy based assay, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. The detection assay may be configured to detect various polymorphism of UGT1A1, and/or the wild type allele, since wild type UGT1A1 is known to properly metabolize SN-38 to SN-38G. The detection assay may be configured to detectin TA repeats in the UGT1A1 promoter region (See, e.g., Invader assays in FIGS. 102, 105 and 106). The detection assay may also be configured to detect cytochrome P-450 3A enzyme polymorphims.

The human wild type UGT1A1 sequence is under accession number NM_{—}000463. There are many polymorphisms in UGT1A1. Below, in Table 12, is a list of fifteen polymorphisms in UGT1A1, along with a reference describing these polymorphism.

TABLE 12

| | |
|---|---|
| 1. | UGT1A1, 13-BP DEL, EX2, see, Ritter et al., J. Clin. Invest. 90: 150-155, 1992, |
| | hereby incorporated by reference. This variant has been designated UGT1A1*2. |
| 2. | UGT1A1, Ser376Phe (C to T transition in Exon 4, see, Bosma, et al., FASEB J. 6: |
| | 2859-2863, 1992, hereby incorporated by reference). This variant has been designated |
| | UGT1A1*3. |
| 3. | UGT1A1, Gln 331Ter (C to T transition, see, Bosma, et al., FASEB J. 6: 2859-2863, |
| | 1992, hereby incorporated by reference). This variant has been designated UGT1A1*5. |
| 4. | UGT1A1, Arg 341Ter (nonsense CGA to TGA mutation, see, Moghrabi et al., |
| | Genomics 18: 171-173, 1993, hereby incorporated by reference). This variant has been |
| | designated UGT1A1*10. |
| 5. | UGT1A1, Gln331Arg (A to G transition, see Moghrabi et al., Genomics 18: 171-173, |
| | 1993, hereby incorporated by reference). This variant has been designated UGT1A1*9. |
| 6. | UGT1A1, Phe170Del (See, Ritter et al., J. Biol. Chem. 268: 23573-23579, 1993, |
| | hereby incorporated by reference). This variant has been designated UGT1A1*13. |
| 7. | UGT1A1, Gly309Glu (G to Transition in codon 309, see, Erps et al., J. Clin. |
| | Invest. 93: 564-570, 1994, hereby incorporated by reference). This variant has been designated |
| | UGT1A1*11. |
| 8. | UGT1A1, 840C to A, Cys-Ter (See, Aono et al., Pediat. Res. 35: 629-632, 1994, |
| | hereby incorporated by reference). This variant has been designated UGT1A1*25. |
| 9. | UGT1A1, Pro229Gln (C to A transition at nucleotide 686, See, Koiwai et al., |
| | Hum. Molec. Genet. 4: 1183-1186, 1995, hereby incorporated by reference. This variant has |
| | been designated UGT1A1*27. Also, see FIG. 101 providing an exemplary INVADER |
| | detection assay design to detect this polymorphism. |
| 10. | UGT1A1, 2-BP insertion “TA” in TATA promoter region (See, Bosma et al., |
| | New Eng. J. Med. 333: 1171-1175, 1995, hereby incorporated by reference. This variant has |
| | been designated UGT1A1*28. Also, see FIG. 102, providing an exemplary INVADER |
| | detection assay design to detect this polymorphism. |
| 11. | UGT1A1, 1-BP insertion, 470T (See, Rosatelli et al., J. Med. Genet. 34: |
| | 122-125, 1997, hereby incorporated by reference). |
| 12. | UGT1A1, IVS1, G-C +1 (G to C mutation at the splice donor site in intron |
| | between exon 1 and exon 2, see, Gantla et al., Am. J. Hum. Genet. 62: |
| | 585-592, 1998, hereby incorporated by reference). |
| 13. | UGT1A1, 145C-T (See, Gantla et al., Am. J. Hum. Genet. 62: |
| | 585-592, 1998, hereby incorporated by reference). |
| 14. | UGT1A1, IVS3, A-G, −2 (See, Gantla et al., Am. J. Hum. Genet. 62: |
| | 585-592, 1998, hereby incorporated by reference). |
| 15. | UGT1A1, Gly71Arg (A to G change at nucleotide 211 in exon 1, see, Akaba et |
| | al., Biochem. Molec. Biol. Int. 46: 21-26, 1998, hereby incorporated by reference). Also, see |
| | FIG. 100, providing an exemplary INVADER detection assay design to detect this |
| | polymorphism. |

Another set of nine polymorphisms in UGT1A1 is provided in FIG. 103. Exemplary detection assays (INVADER assays) for these nine polymorphisms are provided in FIG. 104, although any type of detection assay may be employed to detect these polymorphisms.

In some embodiments, the present invention provides methods for selecting therapy for a subject, comprising; a) providing; i) a sample from the subject, and ii) a detection assay configured to detect a polymorphism in a gene sequence associated with Irinotecan safety or efficacy, b) contacting the sample with the detection assay under conditions such that the presence or absence of the polymorphism in the gene sequence is determined, and c) identifying the subject as suitable for treatment with Irinotecan based on the absence of the polymorphism in the gene sequence; or identifying the subject as not suitable for treatment with Irinotecan based on the presence of the polymorphism in the gene sequence. In other embodiments, the methods further comprise step d) administering Irinotecan to the subject identified as suitable for treatment with Irinotecan. In certain embodiments, the methods further comprise step d) informing the subject that they have been identified as not suitable for treatment with Irinotecan.

In some embodiments, the gene sequence associated with Irintoecan safety or efficacy is UGT1A1 (e.g. human UGT1A1). In other embodiments, the polymorphism in the gene associated with Irinotecan safety or efficacy is selected from a UGT1A1 polymorphism listed in Table 12, or a UGT1A1 polymorphism listed in FIG. 103. In particular embodiments, the gene sequence associated with Irinotecan safety or efficacy is an P-450 3A enzyme. In preferred embodimnts, the polymorphism is a repeat sequence (e.g. TA repeat) in the promoter region of the UGT1A1 gene (e.g. a 5 repeat, 6 repeat, 7 repeat, or 8 repeat). In other preferred embodiments, the repeats are detected with the INVADER assay (See, e.g., FIGS. 102, 105, and 106).

In certain embodiments, the subject has been diagnosed with cancer. In other embodiments, the cancer is colorectal cancer. In additional embodiments, the sample from the subject is a blood sample, urine sample, semen sample, skin sample, or hair sample. In some embodiments, the detection assay is selected from a TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments, the detection assay is an INVADER detection assay. In particularly preferred embodiments, the INVADER detection assay is selected from those shown in FIG. 104.

In certain embodiments, the sample is also screened with a detection assay to determine if the subject will benefit from a second drug that counteract side-effects of Irinotecan administration (exampled of second drugs include, but are not limited to, atropine, loperamide, and antimetics). In other embodiments, the side effects are selected from early-onset diarrhea, contraction of pupils, lacrimation, flushing, rhinitis, increased salivation, diaphoresis, abdominal cramping, late-onset diarrhea, nausea, vomiting, myelosuppression, and sepsis. In certain embodiments, the subject is administered Irinotecan and a second drug to counteract the side effects of the Irinotecan administration.

In some embodiments, the detection assay is located on a panel (e.g. a detection panel configured to detect at least one UGT1A1 polymorphism shown in FIG. 103). In other embodiments, the conditions in the contacting step comprises performing a mutiplexed PCR amplification reaction.

In certain embodiments, the present invention provides methods for selecting therapy for a subject, comprising; a) providing; i) a sample from the subject, and ii) a detection panel comprising at least two unique detection assays, wherein each of the at least two unique detection assays is configured to detect a polymorphism in a gene sequence associated with Irinotecan safety or efficacy, b) contacting the sample with the detection panel under conditions such that each of the at least two unique detection assays reveals the presence or absence of a polymorphism, and c) identifying the subject as suitable for treatment with Irinotecan based on the absence of polymorphisms detected by the at least two detection assays, or identifying the subject as not suitable for treatment with Irinotecan based on the presence of at least one polymorphism detected by the at least two detection assays. In some embodiments, the methods further comprise step d) administering Irinotecan to the subject identified as suitable for treatment with Irenotecan. In other embodiments, the methods further comprise step d) informing the subject that they have been identified as not suitable for treatment with Irenotecan.

In particular embodiments, each of the at least two unique detection assays is configured to detect a polymorphism in the UGT1A1 gene. In preferred embodiments, each of the at least two unique detection assays is configured to detect a polymorphism selected from a UGT1A1 polymorphism listed in Table 12, or a UGT1A1 polymorphism listed in FIG. 103. In particularly preferred embodiments, at least one of the detection assays is selected from a UGT1A1 polymorphism listed in FIG. 103. In other embodiments, at least one of the detection assay is configured to detect a polymorphism is an P-450 3A enzyme.

In certain embodiments, the subject has been diagnosed with cancer. In other embodiments, the cancer is colorectal cancer. In some embodiments, the sample from the subject is a blood sample, uirine sample, semen sample, skin sample, or hair sample. In certain embodiments, at least one of the at least two detection assays is selected from a TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments; at least one of the detection assays is an INVADER detection assay. In particularly preferred embodiments, the INVADER detection assay is selected from those shown in FIG. 104.

In certain embodiments, the sample is also screened with a detection assay to determine if the subject will benefit from a second drug that counteracts side effects of Irinotecan administration. Examples of second drugs include, but are not limited to, atropine, loperamide, and antimetics. In other embodiments, the side effects are selected from early-onset diarrhea, contraction of pupils, lacrimation, flushing, rhinitis, increased salivation, diaphoresis, abdominal cramping, late-onset diarrhea, nausea, vomiting, myelosuppression, and sepsis.

In particular embodiments, the subject is administered irinotecan and a second drug to counteract the side effects of the Irinotecan administration. In other embodiments, the conditions in the contacting step comprises performing a mutiplexed PCR amplification reaction.

In some embodiments, the present invention provides kits comprising; a) a detection assay configured to detect a polymorphism in a gene sequence associated with Irinotecan safety or efficacy, and b) written component, wherein the written component comprises instructions for identifying if a subject is suitable for treatment with Irinotecan based on the results of employing the detection assay on a sample from the patient. In other embodiments, the present invention provides kits comprising; a) a detection assay configured to detect a polymorphism in a gene sequence associated with Irinotecan safety or efficacy, and b) a composition comprising Irinotecan.

In certain embodiments, the present invention provides methods of marketing, comprising; advertising the sale of Irinotecan and a detection assay configured to detect a polymorphism in a gene sequence associated with Irinotecan safety or efficacy together. In other embodiments, the present invention provides methods comprising; a) designing a detection assay to detect a polymorphism associated with Irinotecan safety or efficacy in a subject, and b) drafting a patent application based on the combination of the detection assay and drug. In other embodiments, the methods further comprise filing the patent application in the United States Patent and Trademark Office. In some embodiments, the present invention provides a patent resulting from the above methods.

The present invention provides methods for detecting polymorphisms in genes affecting the action of therapeutic agents. In some embodiments, detection of a single polymorphism, or any combinations of polymorphisms can be performed on amplification products, including cDNA, run-off transcripts, genomic DNA or RNA. In another embodiment, the method includes detection of polymorphisms known to occur more frequently in a particular ethnic group, gender, or age group, and which are associated with therapeutic dosing decisions for, or adverse reactions to a particular therapeutic agent. In a preferred embodiment, a universal kit would be used to screen for any polymorphism that correlates with an adverse reaction to, a therapeutic agent for, any ethnicity, gender, or age. In another embodiment, the universal test screens for polymorphisms associated with an adverse reaction or therapeutic dosing decision wherein some subset of the universal kit polymorphisms would be used based on phenotypic information such as ethnicity, gender, or age any combination of phenotypic information. In one embodiment, the universal test could be used to screen for all UGT1A1 polymorphisms before drug or therapeutic agent administration. In one preferred embodiment, the universal test could screen for all UGT1A1 polymorphisms before administering irinotecan. In another embodiment, the universal test could screen for all UGT1A1 polymorphisms and additional polymorphisms in other genes that correlated with the benefit or outcome for irinotecan therapy.

Another embodiment of the present invention includes entering phenotypic information such as ethnicity, gender, age, height, weight, etc. into a therapy analysis algorithm, wherein said algorithm may utilize any one or combination of these phenotypic parameters along with any one or combination of genotypic results included in the universal test to determine the patient's therapy. It is envisioned that one or more polymorphisms may provide a means for sub-selection of data from another set of polymorphisms that could be utilized for therapeutic decisions such as whether to use a particular therapeutic agent and at what dosing level or if another therapeutic agent is recommended. In another embodiment, one or more phenotypic parameters may provide a means for sub-selection of data from a set of polymorphisms that could be utilized for therapeutic decisions.

In some embodiments, the present invention provides a universal test for all SNPs associated with a drug response of any type. For example there may be 10 SNPs. One SNP could be the only marker needed or several may be needed to determine if the person will have an adverse drug reaction. Depending on phenotypic information, any one or combination of SNPs may mean something different. For example, 1 SNP may be the best indicator of whether to take irinotecan if you are a woman, but another SNP may be the best indicator if you are a man. In another embodiment, it may be best not to rely on the phenotypic information being given in order to make the therapeutic decision. For example, ethnicity determination may be difficult to assess. In this case the universal test (testing for all SNPs associated with a drug response) would be able to provide the needed result even though all those SNPs for any one individual may not be necessary.

In some embodiments, the present invention provides systems for manufacturing and/or selling pharmacogenetic detection assays, comprising: a) a pharmacogenetic detection assay production component for creating a UGT polymorphism detecting pharmacogenetic oligonucleotide detection assay; b) a pharmacogenetic detection assay quality control component; and c) a label generator, wherein the label generator comprises a device for providing indicia on a package or package insert related to the UGT polymorphism detecting pharmacogenetic detection assay, wherein the indicia is selected from the group consisting of intended use indicia, patient population indicia, proprietary name indicia, established name indicia, quantity indicia, concentration indicia, source indicia, measure of activity indicia, warning indicia, precaution indicia, storage instruction indicia, reconsitution indicia, expiration date indicia, observable indication of alteration indicia, net quantity of contents indicia, number of tests indicia, manufacturer indicia, packer indicia, distributor indicia, lot number indicia, control number indicia, chemical principle indicia, physiological principle indicia, biological principle indicia, mixing instruction indicia, sample preparation indicia, use of instrumentation indicia, calibration indicia, specimen collection indicia, known interfering substances indicia, step by step outline of recommended procedures from reception of specimen to result indicia, indicia indicative for improving performance, indicia indicative for improving accuracy, list of materials indicia, amount indicia, time indicia used to assure accurate results, positive control indicia, negative control indicia, indicia explaining the calculation of an unknown, formula indicia, limitation of procedure indicia, additional testing indicia, range of expected value indicia, specificity indicia, sensitivity indicia, pertinent reference indicia, batch indicia, and date of issuance of last revision of label indicia.

In some embodiments, the storage instruction indicia is selected from the group consisting of temperature indicia, and humidity indicia. In other embodiments, the system further comprises a device for providing multiple container packaging for the pharmacogenetic oligonucleotide detection assay. In other embodiments, the quality control component comprises an electronic document control component. In particular embodiments, the quality control component further comprises a purchasing control component.

In some embodiments, the quality control component further comprises a vendor ranking component. In other embodiments, the vendor ranking component comprises a vendor quality ranking component. In certain embodiments, the system further comprises a database of acceptable supplier, contractors, and consultants. In particular embodiments, the quality control component comprises a database comprising electronic purchasing documents. In other embodiments, the system further comprises a product identifier component. In some embodiments, the product identifier component comprises a system for identifying a pharmacogenetic olionucleotide detection assay or components thereof through a stage, the stage selected from the group consisting of a receipt stage, production stage, distribution stage, and installation stage. In certain embodiments, the product identifier component comprises a fail safe anti-mix up module. In some embodiments, the quality control component further comprises a contamination control component.

In particular embodiments, the quality control component comprises validated computer software. In other embodiments, the quality control component further comprises electronic calibration records for one or more components of the system. In some embodiments, the quality control component further comprises a non-conforming pharmacogenetic oligonucleotide detection assay rejection component. In other embodiments, the non-conforming product rejection component further comprises a system for evaluation, segregation and disposition of non-conforming pharmacogenetic oligonucleotide detection assay rejection component. In some. embodiments, the production component communicates with the quality control component. In further embodiments, the communication comprises a non-conformance notifier.

In other embodiments, the quality control component further comprises statistical routines to detect a quality problem with the pharmacogenetic oligonucleotide detection assay. In certain embodiments, the system further comprises a pharmacogenetic oligonucleotide detection assay device master recorder. In some embodiments, the system further comprises a pharmacogenetic oligonucleotide detection assay device history recorder. In additional embodiments, the device history recorder comprises data of a detection assay or batch manufacture date, quantity data, quality data, acceptance record data, primary identification label data, and control number data. In other embodiments, the system further comprises a quality system recorder. In yet other embodiments', the system further comprises a complaint file recorder.

In particular embodiments, the system further comprises a pharmacogenetic oligonucleotide detection assay tracker. In other embodiments, the pharmacogenetic detection assay a detection assay capable of detecting one or more TA repeats in a promoter of the gene. In some embodiments, the pharmacogenetic detection assay is a detection assay capable of detecting five or more TA repeats in a promoter Of the gene. In other embodiments, the pharmacogenetic detection assay is a detection assay capable of detecting eight or more TA repeats in a promoter of the gene.

In some embodiments, the pharmacogenetic detection assay comprises a plurality of detection assays capable of detecting gene expression or more than one polymorphisms across different ethnic groups. In additional embodiments, the different ethnic groups are selected from the group consisting of an African American ethnic group, an asian ethnic group, a hispanic ethnic group, and a Caucasian ethnic group.

In other embodiments, the more than one polymorphisms in UGT1A1 are selected from the group of one or more TA repeats in a promoter region of the gene. In some embodiments, the production component is configured to produce or inventory substantially similar batch quantities of two or more detection assays or detection assay components. In particular embodiments, the detection assays are selected from the group consisting of detection assays configured to detect TA repeats in a promoter region of the gene, in combination with one or more exonic polymorphisms. In other embodiments, the pharmacogenetic detection assay is a detection assay capable of detecting gene expression of a UGT1A1 gene, and one or more TA repeats in a promoter of the gene, and one or more exonic polymorphisms in the gene. In further embodiments, the pharmacogenetic detection assay is a detection assay capable of detecting gene expression of a UGT1A1 gene comprising one or more polymorphisms, the polymorphisms selected from the group consisting of promoter region polymorphisms and exonic polymorphisms. In some embodiments, the pharmacogenetic detection assay is a detection assay capable of detecting gene expression of a gene. In other embodiments, the pharmacogenetic detection assay is a detection assay capable of detecting gene expression of a gene across more than one ethnic group. In particular embodiments, the pharmacogenetic detection assay is a detection assay capable of detecting gene expression of a gene across all ethnic groups.

In some embodiments, the detection assay comprises a hybridization assay. In other embodiments, the detection assay comprises a TAQMAN assay. In other embodiments, the detection assay comprises an invasive cleavage assay. In further embodiments, the detection assay comprises mass spectroscopy. In other embodiments, the detection assay comprises microarray. In other embodiments, the detection assay comprises a polymerase chain reaction. In further embodiments, thethe detection assay comprises a rolling circle extension assay. In some embodiments, the detection assay comprises a sequencing assay.

In particular embodiments, the detection assay comprises a hybridization assay employing a probe complementary to a polymorphism. In other embodiments, the detection assay comprises a bead array assay. In some embodiments, the detection assay comprises a, primer extension assay. In additional embodiments, the detection assay comprises an enzyme mismatch cleavage assay. In particular embodiments, the detection assay comprises a branched hybridization assay. In other embodiments, the detection assay comprises a NASBA assay. In further embodiments, the detection assay comprises a molecular beacon assay. In still other embodiments, the detection assay comprises a cycling probe assay. In some embodiments, the detection assay comprises a ligase chain reaction assay. In other embodiments, the detection step comprises a sandwich hybridization assay.

In some embodiments, the system further comprises a drug production or drug inventory level monitoring device for a drug, whereby production or inventory of the pharmacogenetic detection assay is adjusted upward or downward based upon data transmitted from the monitoring device. In further embodiments, the drug is irinotecan or a derivative thereof, and in which the pharmacogenetic detection assay is capable of determining the presence or absense of one or more drug metabolism markers, the markers selected from the group consisting of UGT 1A1 promoter region polymorphisms, and UGT 1A1 exonic polymorphisms.

In some embodiments, the present invention provides pharmacogenetic detection assay kits (e.g. created via the systems above). In further embodiments, the detection assay comprises a hybridization assay. In other embodiments, the detection assay comprises a TAQMAN assay. In some embodiments, the detection assay comprises an invasive cleavage assay. In other embodiments, the detection assay comprises mass spectroscopy, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, or a sandwich hybridization assay.

The present invention also provides pharmacogenetic detection assay kits created by the system described above in which the pharmacogenetic detection assay is capable of detecting one or more polymorphisms in UGT1A1. In some embodiments, the polymorphisms are associated with metabolism of camptothecin, or a derivative thereof. In other embodiments, the camptothecin derivative is Topotecan or Irinotecan. In some embodiments, the camptothecin derivative is Irinotecan. In additional embodiments, the detection assay comprises a hybridization assay. In other embodiments, the detection assay comprises a TAQMAN assay, an invasive cleavage assay, mass spectroscopy, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, or a sandwich hybridization assay.

In other embodiments, the kit further comprises a drug. In some embodiments, the drug comprises camptothecin, or a camptothecin derivative. In some embodiments, the camptothecin derivative is Topotecan or Irinotecan. In certain embodiments, the camptothecin derivative is Irinotecan. In some embodiments, the drug is irinotecan or a derivative thereof; and in which the pharmacogenetic detection assay is capable of determining the presence or absense of one or more drug metabolism markers, the markers selected from the group consisting of UGT 1A1 *6, *7, *27, *28, and *29.

In other embodiments, the present invention provides universal pharmacogenetic detection assay kits, in which the pharmacogenetic detection kit members are capable of detecting two or more polymorphisms prevalent in three or more ethnic groups. In some embodiments, the ethnic groups are selected from the group consisting of African Americans, Caucasians, Asians, Europeans, and Indian Americans.

In some embodiments, the present invention provides pharmacogenetic detection assay kits, in which the pharmacogenetic detection kit members are capable of detecting polymorphisms in a UGT gene, the kit capable of detecting polymorphisms common in more than one ethnic group and, in which the polymorphisms have a threshold allelle frequency. In certain embodiments, the threshold allelle frequency is greater than about one percent, and in which the kit is capable or detecting two or more polymorphisms common in a first ethnic group, and two or more polymorphisms common in a second ethnic group. In other embodiments, the threshold allelle frequency is greater than about three percent, and in which the kit is capable of detecting X number of polymorphisms common in a first ethnic group, where X is an interger greater than or equal to two, in which the kit is capable or detecting Y number of polymorphisms common in a second ethnic group, where Y is an interger greater than or equal to two, and in which the kit is capable or detecting Z number of polymorphisms common in a third or more ethnic groups, where Z is an interger greater than or equal to two.

In certain embodiments, the threshold allelle frequency is greater than about five percent. In other embodiments, the threshold allelle frequency is greater than about ten percent. In still other embodiments, the threshold allelle frequency is in the range of about 1 percent to about 95 percent. In some embodiments, the polymorphisms are associated with metabolism of camptothecin, or a derivative thereof. In further embodiments, the camptothecin derivative is Topotecan or Irinotecan. In other embodiments, the camptothecin derivative is Irinotecan.

In some embodiments, the kit further comprises a drug. In other embodiments, the drug comprises camptothecin, or a camptothecin derivative. In certain embodiments, the camptothecin derivative is Topotecan or Irinotecan. In additional embodiments, the camptothecin derivative is Irinotecan.

In some embodiments, the present invention provides pharmacogenetic detection assay kits in which the pharmacogenetic detection kit members are capable of detecting polymorphisms related to more than one ethnic group and, in which the polymorphisms have a threshold allelle frequency.

In some embodiments, the present invention provides methods for detecting polymorphisms in a uridine diphosphate glucuronosyl transferase (UGT) gene promoter comprising determining the presence or absence of at least five or greater (TA) repeats in the promoter with a non-amplified oligonucleotide detection assay. In certain embodiments, the methods comprise the steps of (a) obtaining DNA or RNA from an individual; and, (b) determining the number of TA repeats in the promoter. In particular embodiments, the promoter is the UGT1A1 promoter. In certain embodiments, the method further comprises amplifying DNA other than all or part of the UGT1A1 promoter DNA in a multiplexed amplification step. In particular embodiments, the promoter has a genotype selected from the group consisting of [TA]₅/[TA]₅, [TA]₅/[TA]₆, [TA]₅/[TA]₇, [TA]₅/[TA]₈ and [TA]₈/[TA]₈.

In certain embodiments, the present invention provides methods for optimizing drug dosages for a patient wherein the drugs are glucuronidated by a uridine diphosphate glucuronosyltransferase (UGT), determining the number of thymidine-adenine (TA) repeats in a promoter of the UGT gene by a non-amplified oligonucleotide detection assay, and up or down dosing the patient based upon the determination. In some embodiments, the non-amplified oligonucleotide detection assay is capable of detecting polymorphisms prevalent above a threshold allele frequency in more than one ethnic group. In additional embodiments, the threshold allelle frequency is greater than about one percent. In other embodiments, the threshold allelle frequency is greater than about three percent. In further embodiments, the threshold allelle frequency is greater than about five percent. In other embodiments, the threshold allelle frequency is greater than about ten percent. In some embodiments, the threshold allelle frequency is in the range of about 1 percent to about 95 percent.

In some embodiments, the non-amplified oligonucleotide detection assay is capable of detecting polymorphisms prevalent in Asians, African Americans, Hispanics and Caucasians. In other embodiments, the promoter has a genotype selected from the group consisting of [TA]₅/[TA]₅, [TA]₅/[TA]₆, [TA]₅/[TA]₇, [TA]₅/[TA]₈, [TA]₆/[TA]₈, [TA]₇/[TA]₈ and [TA]₈/[TA]₈.

In certain embodiments, the present invention provides methods for optimizing drug dosages for a patient wherein the drugs are glucuronidated by a uridine diphosphate glucuronosyltransferase (UGT), determining the number of thymidine-adenine (TA) repeats in a promoter of a UGT gene by a universal oligonucleotide detection assay capable of detecting two or more genetic polymorphisms across two or more ethnic groups. In other embodiments, the method further comprises up or down dosing the patient based upon the determination. In particular embodiments, the universal oligonucleotide detection assay is capable of detecting three or more genetic polymorphisms across three or more ethnic groups. In some embodiments, the method further comprises monitoring gene expression of the UGT gene.

In some embodiments, the present invention provides methods for optimizing drug dosages for a patient wherein the drugs are glucuronidated by a uridine diphosphate glucuronosyltransferase (UGT), determining gene expression of the UGT gene by an oligonucleotide detection assay capable of detecting gene expression of the UGT gene. In other embodiments, the method further comprises up or down dosing the patient based upon the determination. In some embodiments, the oligonucleotide detection assay is capable of detecting gene expression in two or more ethnic groups.

In some embodiments, the present invention provides kits for optimizing drug dosages for a patient wherein the drugs are glucuronidated by a uridine diphosphate glucuronosyltransferase (UGT), comprising oligonucleotide detection assay components capable of detecting gene expression of the UGT gene. In other embodiments, the oligonucleotide detection assay components are capable of detecting gene expression in two or more ethnic groups. In some embodiments, the kit further comprises a drug. In particular embodiments, the drug comprises camptothecin, or a camptothecin derivative. In some embodiments, the camptothecin derivative is Topotecan or Irinotecan. In certain embodiments, the gene further comprises a gene promoter, the promoter of the gene having a genotype selected from the group consisting of [TA]5/[TA]5, [TA]5/[TA]6, [TA]5/[TA]7, [TA]5/[TA]8, [TA]6/[TA]8, [TA]7/[TA]8 and [TA]8/[TA]8.

Detection of UGT1A1 Dinucleotide Repeat Polymorphism *28

The hepatic uridine diphosphate glucuronosyltransferase (UGT) 1A1 enzyme is responsible for the conjugation and detoxification of SN-38, the active form of irinotecan. Irinotecan is an anticancer drug used in the treatment of colorectal and lung cancers. Mutations in the UGT1A1 gene cause altered (e.g., reduced or increased) enzymatic activity. Reduced activity can lead to toxicity due to the excessive accumulation of SN-38, resulting in diarrhea and leukopenia. A TA insertion in the highly repetitive TATA-box of the gene promoter is the most common type of UGT1A1 variant. The wild-type allele is referred to as (TA)6. The (TA)7 allele, UGT1A1*28, leads to decreased metabolism of irinotecan and is also associated with Gilbert's Syndrome, a benign form of unconjugated bilirubinemia.

The embodiments described below provide assays designed to distinguish between wild-type (TA)6 and insertion mutation (TA)7 sequences and to detect the deletion of a TA, (TA)5, and the insertion of two TA repeats, (TA)8. The designs provided here can be adapted to the detection of any of these variants.

In the preferred embodiments described herein, the probe set targets a T within the TA repeat region of the antisense strand, such that most of the (TA)6 and (TA)7 differentiation comes from the analyte-specific region of the probes. Embodiments of this design are shown in FIGS. 105, 106, 109, 111, 112, and 121. In the embodiment shown in FIG. 105, the wild-type probe uses the arm termed “ER38” and is reported by a FRET cassette having the REDMOND RED dye (“Red dye,” Synthetic Genetics, San Diego, Calif.). The insertion probe shown in this embodiment uses the “ER24” arm and is reported by a FRET cassette having the Fam (fluorescein) dye. In an alternative embodiment diagrammed in FIGS. 106 and 121, the WT probe uses the “DM” arm. In preferred embodiments, the probes and the FRET cassettes are blocked on the 3′ ends with hexanediol. In some embodiments, the detection assays are designed to detect (TA)5 and (TA)8. Embodiments of this type of design are shown in 109, 110, and 113.

By way of example, and not intending to limit the procedures of the present invention to any particular configuration or combination of components, the following section describes certain embodiments of a procedure for practicing the present invention:

### UGT EXAMPLE 1

Reaction Set-Up:

Place 10 ul of sample or control in reaction well.

Overlay with 20 ul Mineral Oil.

Heat to 95C for 5 minutes to denature.

Cool to 63C for Reaction Mix addition.

Add 10 ul INVADER Reaction Mix (see below) to each well and mix (e.g., by pipetting).

Incubate at 63C for 4 hours.

Cool to 4C to await fluorescence reading.

Warm to room temperature.

Scan in fluorescence plate reader.

INVADER Reaction Mix (Per Reaction):

5 ul DNA Reaction Buffer 1 (14% PEG, 10 mM MOPS pH 7.5, 56 mM MgCl2, 0.02%

ProClin 300)

1 ul 1 uM Invader Oligo (in Te)

1 ul 10 uM each WT and Mut Probes (in Te)

1 ul 5 uM Fam FRET (in Te)

1 ul 5 uM Red FRET (in Te)

1 ul 40 ng/ul Cleavase X (in Cleavase Dilution Buffer) 4

Final Reaction Concentrations:

3.5% PEG

10 mM MOPs

1.0 pmol INVADER oligonucleotide

10 pmol each primary probe

5 pmol each FRET

40 ng Cleavase X (Third Wave Technologies, Madison, Wis.)

14 mM MgCl2

The results of tests run under these conditions are shown in FIG. 107. DNA samples 14641, 14640 and 1600 were purchased from Coreill Institute for Medical Research (Camden, N.J.). The remaining DNA samples were prepared in house using the Gentra PureGene DNA extraction method.

To assess the sensitivity of the assay, DNA samples were tested at concentrations of 10 to 500 ng, with the results diagrammed in FIG. 108. The LOD for each sample was determined by t-test vs. 0, Ratio, and FOZ. The LOD for the wild-type sample was 10 ng by t-test and by Ratio, but 20 ng by FOZ. The highest level of cross-reactivity was 1.11 FOZ. This small amount of cross reactivity did not interfere with the genotype call by the INVADER assay. The heterozygous sample had a LOD of 10 ng by t-test and by Ratio, and 50 ng by FOZ. The Het Ratio increased slightly from 0.95 to 1.09 as the amount of DNA increased. There was no cross-reactivity with the wild-type probe on the insertion target. The LOD for the insertion sample by t-test was 10 ng, by FOZ was 50 ng, and by Ratio was 20 ng. There was no cross-reactivity with the wild-type probe one the insertion target.

These data demonstrate the application of the INVADER assay to the detection of polymorphisms comprising short tandem repeat sequences.

### UGT Example 2

### TA5 and TA8 INVADER Assays

The example describes performing TA5 and TA8 UGT1A1 detection with the INVADER assay. The INVADER assay design for TA5 in this example is shown in FIG. 110 and the INVADER assay design for TA8 in this example is shown in FIG. 113. The TA5 and TA8 monoplex assays were run across the same set of genomic samples and synthetic targets. In both cases, the probes reported to Fam dye. The following assay conditions were employed:

ASR 10:10 Reaction Format:

Place 10 ul of sample or control in reaction well.

Overlay with 20 ul Mineral Oil.

Heat to 95C for 5 minutes to denature.

Cool to 63C for Reaction Mix addition.

Add 10 ul Invader Reaction Mix (see below) to each well; mix by pipetting.

Incubate at 63C for 4 hours.

Cool to 4C to await fluorescence reading.

Warm to room temperature.

Scan in fluorescence plate reader

Invader Reaction Mix (Per Reaction):

5 ul DNA Reaction Buffer 1 (14% PEG, 40 mM MOPS pH 7.5, 56 mM MgCl2, 0.02%

ProClin 300)

1 ul 10 uM Invader Oligo (in Te)

1 ul 10 uM UGT1A1*28 probe (in Te)

0.5 ul 10 uM Fam FRET(in Te)

1 ul 40 ng/ul Cleavase X (in Cleavase Dilution Buffer)

1.5 ul water

Final Reactions Concentrations:

3.5% PEG

1 mM MOPs

1.0 pmol Invader

10 pmol primary probe

5 pmol FRET

40 ng Cleavase X

14 mM MgCl2

The first three samples were run to test for cross-reactivity. Both the TA5 and TA8 assays were run with a TA6/TA7 genomic Het sample, 38838. The TA5 assay was tested on the TA8 synthetic target, and the TA8 assay was tested on the TA5 synthetic target. The TA5 probe only produced signal with the TA5 target. The TA8 probe only produced signal with the TA8 target. There was no cross-reactivity with the genomic sample 38838. This indicates that the TA5 probe does not cross-react with the TA6, TA7, TA8 sequences, and the TA8 probes does not cross-react with the TA5, TA6, TA7 sequences (See, FIG. 115A).

The remaining genomic DNAs were screened for the TA5 and TA8 alleles. Samples 03-237, 03-265, 03-276, 03-313, 03-364 showed signal with the TA5 assay (See, FIG. 115B). Samples 03-265 and 03-318 showed signal with the TA8 assay (See, FIG. 115B).

The six genomic samples that showed signal in the TA5 and TA8 assays were run in the TA5 and TA8 assays were then run in the TA6/TA7 biplex assay. The UGT1A1*28 INVADER genotypes for these six samples is shown below.

| Sample | UGT1A1*28 Genotype |
|---|---|
| 03-237 | TA5/TA6 |
| 03-265 | TA5/TA8 |
| 03-276 | TA5/TA7 |
| 03-313 | TA5/TA7 |
| 03-318 | TA6/TA8 |
| 03-364 | TA5/TA6 |

The set up for TA6/TA7 biplex assay was as follows.
ASR 10:10 Reaction Format:

Place 10 ul of sample or control in reaction well.

Overlay with 20 ul Mineral Oil.

Heat to 95C for 5 minutes to denature.

Cool to 63C for Reaction Mix addition.

Add 10 ul Invader Reaction Mix (see below) to each well;

mix by pipetting.

Incubate at 63C for 4 hours.

Cool to 4C to await fluorescence reading.

Warm to room temperature.

Scan in fluorescence plate reader.

Invader Reaction Mix (Per Reaction):

5 ul DNA Reaction Buffer 1 (14% PEG, 40 mM MOPS pH 7.5, 56 mM MgCl2, 0.02%

ProClin 300)

1 ul 1 uM Invader Oligo (in Te)

1 ul 10 uM each WT and Mut Probes (in Te)

1 ul 5 uM Fam FRET (in Te)

1 ul 5 uM Red FRET (in Te)

1 ul 40 ng/ul Cleavase X (in Cleavase Dilution Buffer)

Final Reactions Concentrations:

3.5% PEG

10 mM MOPs

1.0 pmol Invader

10 pmol each primary probe

5 pmol each FRET

40 ng Cleavase X

14 mM MgCl2

The five samples that were positive for either TA5 or TA8 (above) were also positive for either the TA6 or TA7 allele. Sample 03-265 was positive for both TA5 and TA8. In the TA6/TA7 assay, this sample resulted in no signal (See, FIG. 116A-B). This indicates that the TA6 and TA7 probes are not cross-reactive with the TA5 or TA8 sequences.

### UGT EXAMPLE 3

### UGT1A1*28 Biplexed with Internal Control

This example describes one embodiment for a UGT1A*28 Assay with an Internal Control. The assay may be designed as a 4 well assay in which each *28 probe (TA5, TA6, TA7, and TA8) are biplexed with an internal control. This assay may employ the INVADER assay for one or more of the *28 probes. FIG. 109 shows useful INVADER assay configurations for TA5, TA6, TA7 and TA8, that may be biplexed with the Alpha Actin internal control shown in FIG. 114. Other useful INVADER configurations that may be employed are shown in FIG. 110 (TA5), FIG. 111 (TA6), FIG. 112 (TA7), and FIG. 113 (TA8), which may be biplexed with the internal control shown in FIG. 114.

Assay set up conditions that may be employed to set up this 4 well assay are as follows.

ASR 10:10 Reaction Format:

Place 10 ul of sample or control in reaction well.

Overlay with 20 ul Minieral Oil.

Heat to 95C for 5 minutes to denature.

Cool to 63C for Reaction Mix addition.

Add 10 ul Invader Reaction Mix (see below) to each well;

mix by pipetting.

Incubate at 63C for 4 hours.

Cool to 4C to await fluorescence reading.

Warm to room temperature.

Scan in fluorescence plate reader.

Invader Reaction Mix (Per Reaction):

5 ul DNA Reaction Buffer 1 (14% PEG, 40 mM MOPS pH 7.5, 56 mM MgCl2,

0.02% ProClin 300)

0.5 ul 2 uM *28 Invader Oligo (in Te),

0.5 ul 2 uM IC Invader Oligo (in Te)

0.5 ul 20 uM *28 Probe (in Te)

0.5 ul 20 uM IC Probe (in Te)

1 ul 5 uM Fam FRET (in Te)

1 ul 5 uM Red FRET (in Te)

1 ul 40 ng/ul Cleavase X (in Cleavase Dilution Buffer)

Final Reactions Concentrations:

3.5% PEG

10 mM MOPs

1.0 pmol each Invader

10 pmol each primary probe

5 pmol each FRET

40 ng Cleavase X

14 mM MgCl2

As the UGT examples above show, the INVADER assay may be configured to detect repeat sequences. The INVADER assay may be configured to detect repeat sequences in other target nucleic acid sequence (e.g. other drug metabolizing genes) that contain repeat sequences. Preferably, the INVADER assay is employed to detect repeat sequences (e.g. in genomic DNA) that are determined to be associated with a particular condition (e.g. predisposition to disease, altered drug metabolism, etc.). For example, INVADER assays may be configured to detect tandemly repetitive sequences, such as satellites, minisatellites, and microsatellites (See, e.g. Bennet, J. Clin. Pathol: Mol. Pathol., 2000; 53:177-183, herein incorporated by reference in its entirity). INVADER assays may also be configured to detect interspersed repetitive DNA sequences such as SINE (e.g. Alu repeat) and LINES. In certain embodiments, the INVADER assays are configured to detect short tandem repeats (STRs) for applications such as forensics and paternity testing (e.g. Tracey, Croatian Medical Journal, 42(3):233-238, 2001, herein incorporated by reference, see also the Marshfield Clinic web site for lists of target repeat sequences for which INVADER assay may be configured to detect). In other embodiments, INVADER assay are configured to detect repeat sequences in plants (e.g. crop plants).

The UGT repeat detection assays of the present invention may also be used in combination with drug therapy (e.g. irinotecan) and additional treatment and/or diagnostic procedures. For example, this combination of UGT detection assays, drug therapy and additional treatment/diagnostic protocols may be applied to the management of colon cancer or lung cancer. For example, FIGS. 117, 118 and 119 show various colon cancer practice guidelines created by the National Comprehensive Cancer Network and modified by the University of Texas M.D. Anderson Cancer Center (See additional management protocols in Adenis et al., Elec. J. of Oncology, 2001, 1, 83-89, herein incorporated by reference). These colon cancer protocols often call for the administration of irinotecan. As such, employing the UGT detection assays of the present invention at one or more places in these colon cancer management flow charts is a useful step in successful patient care.

iii. Latanoprost

An important, and currently available agent for reducing intraocular pressure (IOP), commonly prescribed for the treatment of glaucoma, is called Latanoprost. Latanoprost is a prostaglandin F2 alpha analogue. Latanoprost's chemical name is isopropyl-(z)-7[1R,2R,3R,5S)3,5-dihydroxy-2-[(3R)-3-hydroxy-5-phenylpentyl]cyclopentyl]-5-hep-tenoate. Latanoprost has a chemical formula of C₂₆H₄₀O₅, and a molecular weight of 432.58. Latanoprost is currently sold under the name XALATAN by Pharmacia & Upjohn Corporation. Latanoprost has been shown to effectively reduce IOP in patients with open-angle glaucoma and ocular hypertension.

Latanoprost is a lipophilic, esterified prodrug, that becomes active when it undergoes enzymatic hydrolysis in the cornea. Studies indicate that ocular responses to latanoprost are mediated by stimulation of the FP receptors. It is believe that Latanoprost lowers IOP by increasing the uveoscleral outflow of aqueous humor (See, Camras et al., Curr Eye Res 1981; 1:205-209, herein incorporated by reference). Topically applied latanoprost absorbs into the cornea and is hydrolyzed to the acid form, which passes into the aqueous humor. It is believed that the acid form is carried into the ciliary muscle, binds to the FP receptors, and initiates a signal cascade in the cell nucleus that induces the transcription of MMP genes (See, Weinreb et al., Prostaglandin effects on the uveoscleral outflow pathway. In: Krieglstein G K (editor). Glaucoma update. Springer Verlag, Berlin, Germany, 1999; 197-202, herein incorporated by reference). Gene products are then converted into active enzymes that initiate degradation of extra-cellular matrix components such as collagens. The resulting reduction of collagnes reduces resistance and facilitates outflow.

Typical dosages of Latanoprost is about one drop in the eyes once daily in the evening. The side effects of Latanoprost administration include a browning of the iris, eyelash changes (e.g. increased length, thickness, pigmentation, and number of lashes), and eyelid skin darkening. Latanoprost is also believe to release isopropanol and methanol when hydrolyzed by endogenous ocular esterases. Isopropanol and methanol are irritants to the eye (See, WO0157015, herein incorporated by reference).

ii. Latanoprost and Nucleic Acid Screening

As mentioned above, Latanoprost is thought to be metabolized by esterases. As such, the present invention provides systems and methods for screening subjects that are candidates for Latanoprost administration, or patients already taking Latanoprost, that include screening subjects for polymorphisms in their esterases. Any type of esterase may be screened for, preferably polymorphisms in ocular esterases are screened. Any type of detection assay may be employed including, but not limited to; a hybridization assay, a TAQMAN assay, or an invasive cleavage assay (e.g. INVADER assay), a mass spectroscopy based assay, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay.

The detection assay may be configured to detect various polymorphism of esterases, such as those polymorphisms in the esterases in Table 10, or be configured to detect the wild type esterase. In certain embodiments, esterases with deleterious polymorphisms (that prevent esterase from functioning) are detected in order to find patients unable to convert Latanoprost into the active form.

TABLE 10

1. CARBOXYLESTERASE 1; CES1 (DNA accession no. NM_{—}001266)
2. ESTERASE D; ESD (DNA accession no. Ml 3450) (See, U.S. Pat. No., 5,011,773, describing various polymorphisms, herein incorporated by reference).
3. PAROXONASE (DNA accession no. NM_{—}000305)
4. ESTERASE A-4; ESA4
5. ESTERASE B3
6. ESTERASE B; ESB
7. ESTERASE C; ESC
8. ESTERASE A-5; ESA5
9. Additional esterases and polymorphisms in Esterases described in US App. No. 20010034023 to Stanton et al., herein incorporated by reference).

In some embodiments, the present invention provides methods for selecting therapy for a subject, comprising; a) providing; i) a sample from the subject, and ii) a detection assay configured to detect a polymorphism in a gene sequence associated with Latanoprost safety or efficacy, b) contacting the sample with the detection assay under conditions such that the presence or absence of the polymorphism in the gene sequence is determined, and c) identifying the subject as suitable for treatment with Latanoprost based on the absence of the polymorphism in the gene sequence; or identifying the subject as not suitable for treatment with Latanoprost based on the presence of the polymorphism in the gene sequence. In other embodiments, the methods further comprise step d) administering Latanoprost to the subject identified as suitable for treatment with Latanoprost. In certain embodiments, the methods further comprise step d) informing the subject that they have been identified as not suitable for treatment with Latanoprost.

In some embodiments, the gene sequence associated with Latanoprost safety or efficacy is a human esterase (e:g. an esterase described in Table 10). In certain embodiments, the subject has been diagnosed with glaucoma or ocular hypertension. In additional embodiments, the sample from the subject is a blood sample, urine sample, semen sample, skin sample, or hair sample. In some embodiments, the detection assay is selected from a TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments, the detection assay is an INVADER detection assay.

In certain embodiments, the sample is also screened with a detection assay to determine if the subject will benefit from a second drug that counteract side-effects of Latanoprost administration. In other embodiments, the side effects are selected from include a browning of the iris, eyelash changes (e.g., increased length, thickness, pigmentation, and number of lashes), eyelid skin darkening, and formation of isopropanol and methanol in the eye. In certain embodiments, the subject is administered Latanoprost and a second drug to counteract the side effects of the Latanoprost administration.

In some embodiments, the detection assay is located on a panel. In other embodiments, the conditions in the contacting step comprises performing a mutiplexed PCR amplification reaction.

In certain embodiments, the present invention provides methods for selecting therapy for a subject, comprising; a) providing; i) a sample from the subject, and ii) a detection panel comprising at least two unique detection assays, wherein each of the at least two unique detection assays is configured to detect a polymorphism in a gene sequence associated with Latanoprost safety or efficacy, b) contacting the sample with the detection panel under conditions such that each of the at least two unique detection assays reveals the presence or absence of a polymorphism, and c) identifying the subject as suitable for treatment with Latanoprost based on the absence of polymorphisms detected by the at least two detection assays, or identifying the subject as not suitable for treatment with Latanoprost based on the presence of at least one polymorphism detected by the at-least two detection assays. In some embodiments, the methods further comprise step d) administering Latanoprost to the subject identified as suitable for treatment with Latanoprost. In other embodiments, the methods further comprise step d) informing the subject that they have been identified as not suitable for treatment with Latanoprost.

In particular embodiments, each of the at least two unique detection assays is configured to detect a polymorphism in a human esterase (See, e.g. Table 10). In preferred embodiments, each of the at least two unique detection assays is configured to detect a polymorphism selected from a human esterase polymorphism in Table 10.

In certain embodiments, the subject has been diagnosed with glaucoma or ocular hypertension. In additional embodiments, the sample from the subject is a blood sample, urine sample, semen sample, skin sample, or hair sample. In certain embodiments, at least one of the at least two detection assays is selected from a TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay.

In certain embodiments, the sample is- also screened with a detection assay to determine if the subject will benefit from a second drug that counteract side-effects of Latanoprost administration. In other embodiments, the side effects are selected from include a browning of the iris, eyelash changes (e.g. increased length, thickness, pigmentation, and number of lashes), eyelid skin darkening, and formation of isopropanol and methanol in the eye. In certain embodiments, the subject is administered Latanoprost and a second drug to counteract the side effects of the Latanoprost administration. In other embodiments, the conditions in the contacting step comprises performing a mutiplexed PCR amplification reaction.

In some embodiments, the present invention provides kits comprising; a) a detection assay configured to detect a polymorphism in a gene sequence associated with Latanoprost safety or efficacy, and b) written component, wherein the written component comprises instructions for identifying if a subject is suitable for treatment with Latanoprost based on the results of employing the detection assay on a sample from the patient. In other embodiments, the present invention provides kits comprising; a) a detection assay configured to detect a polymorphism in a gene sequence associated with Latanoprost safety or efficacy, and b) a composition comprising Latanoprost.

In certain embodiments, the present invention provides methods of marketing, comprising; advertising the sale of Latanoprost and a detection assay configured to detect a polymorphism in a gene sequence associated with Latanoprost safety or efficacy together. In other embodiments, the present invention provides methods comprising; a) designing a detection assay to detect a polymorphism associated with Latanoprost safety or efficacy in a subject, and b) drafting a patent application based on the combination of the detection assay and drug. In other embodiments, the methods further comprise filing the patent application in the United States Patent and Trademark Office. In some embodiments, the present invention provides a patent resulting from the above methods.

iv. Celecoxib

An important, and currently available arthritis medication, is called Celecoxib. Celecoxib's chemical formula is 4-[5-(4-methylphenyl)-3-(trifluoromethyl)-H-pyrazol-1-yl]benzenesulfonamide and is a diaryl substituted pyrazole. The empirical formula for Celecoxib is C₁₇H₁₄F₃N₃O₂S. Celecoxib is currently sold under the name CELEBREX by Pharmacia and Pfizer Inc. Celecoxib is used to treat arthritis pain. The mechanism of action is believed to be due to inhibition of prostaglandin synthesis, specifically inhibition of cyclooxygenase-2 (COX-2). Celecoxib is known to be metabolized by P450 2C9 (CYP2C9) into three metabolites (a primary alcohol, a corresponding carboxylic acid and its glucuronide conjugates).

Clinical studies have shown that Celecoxib was able to significantly reduce joint pain in osteroarthritis and rheumatoid arthritis patients, as well as a reduction in pain in analgesic models of post-oral surgery pain, post-orthopedic pain, and primary dysmenorrhea. Celecoxib has also shown effectiveness in treating familial adenomatous polyposis (FAP). Typical oral dosages for humans of Celecoxib suffering from osteoarthritis and rheumatoid arthritis is about 100 mg twice a day, or about 200 mg once a day. For management of acute pain and treatment of primary dysmenorrhea, atypical dosage is about 200 mg twice per day. Finally, for FAP a typical dosage of Celecoxib is about 400 mg (e.g. two 200 mg tables taken twice per day).

Clinical studies with Celecoxib have shown certain side effects. For example, reported side effects include indigestion, diarrhea, and abdominal pain. In more rare circumstances, instances of stomach bleeding have been reported.

ii. Celecoxib and Nucleic Acid Screening

As mentioned above, Celecoxib is known to metabolized by CYP29C. As such, the present invention provides systems and methods for screening subjects that are candidates for Celecoxib administration, or patients already taking Celecoxib. Any type of detection assay may be employed including, but not limited to; a hybridization assay, a TAQMAN assay, or an invasive cleavage assay (e.g. INVADER assay), a mass spectroscopy based assay, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. The detection assay may be configured to detect various polymorphism of CYP2CP, and/or the wild type allele.

The wild type allele, capable of metabolizing Celecoxib, is known as CYP2C9*1 (See, Romkes et al., Biochemistry Apr. 2, 1991;30(13):3247-55, which is hereby incorporated by reference). Two common CYP2CP polymorphisms that reduce the ability of CYP2C9 to function are CYP2CP*2 and CYP2CP*3. The CYP2CP*2 polymorphism is an R144C change in the protein that is caused by a C to T nucleotide change at position 430 (See, Rettie et al., Pharmacogenetics February 1994;4(1):39-42 and Crespi et al., Pharmacogenetics June 1997;7(3):203-10, both of which are hereby incorporated by reference). The CYP2CP*3 polymorphism is an 1359L change in the protein that is caused by an A to C nucleotide change a position 1075 (See, Sullivan-Klose et al., Pharmacogenetics Aug. 1996;6(4):341-9 and Takanashi et al., Pharmacogenetics March 2000;10(2):95-104, both of which are hereby incorporated by reference). Another mutation that causes an amino acid change a position 359 is represented by the CYP2C9*4 polymorphism. The CYP2C9*4 polymorphism is an 1359T change in the protein that is caused by a T to C nucleotide change at position 1076 (See, Imai, et al., Pharmacogenetics February 2000;10(1):85-9, hereby incorporated by reference). Another polymorphism known to cause a decrease in CYP2C9 activity is CYP2C9*5, which is a D360E change in the protein caused by a C to G nucleotide change at position 1080 (See, Dickmann, et al., Mol Pharmacol August 2001;60(2):382-7, hereby incorporated by reference). Another polymorphism that may be detected is CYP2C9*6 which is an 818delA nucleotide change causing a frame shift (See, Kidd et al., Pharmacogenetics December 2001;11(9):803-8, which is hereby incorporated by reference). The present invention also contemplates testing for more than one CYP2C9. For example, two or more of the above polymorphisms may be screened on a single panel. The panels may also include additional detection assays for additional genes affecting the action of CYP2C9, such as the MDR1 gene. For example, the MDR1 C to T polymorphism at position 3435 may also be screened (See, e.g., Kerb et al., The Pharmacogenomics Journal, 2001, 1:204-210, hereby incorporated by reference).

In some embodiments, the present invention provides methods for selecting therapy for a subject, comprising; a) providing; i) a sample from the subject, and ii) a detection assay configured to detect a polymorphism in a gene sequence associated with Celecoxib safety or efficacy, b) contacting the sample with the detection assay under conditions such that the presence or absence of the polymorphism in the gene sequence is determined, and c) identifying the subject as suitable for treatment with Celecoxib based on the absence of the polymorphism in the gene sequence; or identifying the subject as not suitable for treatment with Celecoxib based on the presence of the polymorphism in the gene sequence. In other embodiments, the method further comprises step d) administering Celecoxib to the subject identified as suitable for treatment with Celecoxib. In additional embodiments, the methods further comprise d) informing the subject that they have been identified as not suitable for treatment with Celecoxib.

In particular embodiments, the gene sequence associated with Celecoxib safety or efficacy is CYP2C9. In preferred embodiments, the polymorphism in the gene associated with Celecoxib safety or efficacy is selected from CYP2C9*2, CYP2C9*3, CYP2C9*4, CYP2C9*5, and CYP2C9*6. In other preferred embodiments, at least two, preferably three, or more of the polymorphisms selected from CYP2C9*2, CYP2C9*3, CYP2C9*4, CYP2C9*5, and CYP2C9*6 are detected. In some embodiments, the gene sequence associated with Celecoxib safety or efficacy is MDR1. In certain embodiment, the polymorphism in MDR1 is C3435T.

In certain embodiments, the subject is suffering from Arthritis. In some embodiments, the sample from the subject is a blood sample, urine sample, semen sample, skin sample, or hair sample. In other embodiments, the detection assay is selected from a TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In certain embodiments, the detection assay is an INVADER for a CYP2C9 polymorphism as shown in FIG. 129.

In some embodiments, the sample is also screened with a detection assay to determine if the subject will benefit from a second drug that counteract side-effects of Celecoxib administration. In other embodiments, the side effects are selected from indigestion, diarrhea, and abdominal pain, and stomach bleeding. In certain embodiments, the patient is administered Celecoxib and a second drug to the subject to counteract the side effects of the Celecoxib administration. In some embodiments, the detection assay is located on a panel. In other embodiments, the conditions in the contacting step comprises performing a mutiplexed PCR amplification reaction.

In additional embodiments, the present invention provides methods for selecting therapy for a subject, comprising; a) providing; i) a sample from the subject, and ii) a detection panel comprising at least two unique detection assays, wherein each of the at least two unique detection assays is configured to detect a polymorphism in a gene sequence associated with Celecoxib safety or efficacy, b) contacting the sample with the detection panel under conditions such that each of the at least two unique detection assays reveals the presence or absence of a polymorphism, and c) identifying the subject as suitable for treatment with Celecoxib based on the absence of polymorphisms detected by the at least two detection assays, or identifying the subject as not suitable for treatment with Celecoxib based on the presence of at least one polymorphism detected by the at least two detection assays. In some embodiments, the method further comprises step d) administering Celecoxib to the subject identified as suitable for treatment with Celecoxib. In other embodiments, the methods further comprise step d) informing the subject that they have been identified as not suitable for treatment with Celecoxib.

In some embodiments, each of the at least two unique detection assays is configured to detect a polymorphism in the CYP2C9 gene. In other embodiments, each of the at least two unique detection assays is configured to detect a polymorphism selected from CYP2C9*2, CYP2C9*3, CYP2C9*4, CYP2C9*5, and CYP2C9*6. In further embodiments, at least one of the detection assay is configured to detect a polymorphism in the MDRl gene (e.g. C3435T).

In some embodiments, the subject is suffering from Arthritis. In certain embodiments, the sample from the subject is a blood sample, urine sample, semen sample, skin sample, or hair sample. In further embodiments, at least one of the at least two detection assays is selected from a TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments, at least one of the at least two detection assays is an INVADER assay as shown in FIG. 129.

In additional embodiments, the sample is also screened with a detection assay to determine if the subject will benefit from a second drug that counteract side-effects of Celecoxib administration. In certain embodiments, the side effects are selected from indigestion, diarrhea, and abdominal pain, and stomach bleeding. In additional embodiments, administering a second drug to the subject to counteract the side effects of the Celecoxib administration. In some embodiments, the conditions in the contacting step comprises performing a mutiplexed PCR amplification reaction.

In some embodiments, the present invention provides kits comprising; a) a detection assay configured to detect a polymorphism in a gene sequence associated with Celecoxib safety or efficacy, and b) written component, wherein the written component comprises instructions for identifying if a subject is suitable for treatment with Celecoxib based on the results of employing the detection assay on a sample from the patient. In other embodiments, the present invention provides kits comprising; a) a detection assay configured to detect a polymorphism in a gene sequence associated with Celecoxib safety or efficacy,,and b) a composition comprising Celecoxib.

In certain embodiments, the present invention provides methods of marketing, comprising; advertising the sale of,Celecoxib and a detection assay configured to detect a polymorphism in a gene sequence associated with Celecoxib safety or efficacy together. In some embodiments, the present invention provides methods comprising; a) designing a detection assay to detect a polymorphism associated with drug safety or efficacy in a subject, and b) drafting a patent application based on the combination of the detection assay and drug. In further embodiments, method further comprising step c) filing the patent application in the United States Patent and Trademark Office. In other embodiments, the present invention provides a patent resulting from these methods.

II. Analyte-Specific Reagents

In some embodiments, components of nucleic acid detection assays are sold as analyte specific reagents (ASRs). ASRs are restricted devices under section 520(e) of the Federal Food, Drugs, and Cosmetic Act and 21 CFR 809.30 and are subject to specific restrictions. ASRs may only be sold to “in vitro diagnostic manufacturers”: clinical laboratories regulated under the Clinical Laboratory Improvement Amendments of 1988 (CLIA), as qualified to perform high complexity testing under 42 CFR part 493 or clinical laboratories regulated under VHA Directive 1106 (available from Department of Veterans Affairs, Veterans Health Administration, Washington, DC 20420); and organizations that use the reagents to make tests for purposes other than providing diagnostic information to patients and practitioners (e.g., forensic, academic, research, and other nonclinical laboratories). In addition, ASRs must be labeled in accordance with Sec. 809.10(e). Advertising and promotional materials for ASRs must include the identity and purity (including source and method of acquisition) of the analyte specific reagent and the identity of the analyte; the statement for class I exempt ASR's: “Analyte Specific Reagent. Analytical and performance characteristics are not established”; include the statement for class II or III ASR's: “Analyte Specific Reagent. Except as a component of the approved/cleared test (name of approved/cleared test), analytical and performance characteristics are not established”; and must not make any statement regarding analytical or clinical performance.

Any laboratory that develops an in-house test using the ASR is required to inform the ordering person of the test result by appending to the test report the statement: “This test was developed and its performance characteristics determined by (Laboratory Name). It has not been cleared or approved by the U.S. Food and Drug Administration.” This statement would not be applicable or required when test results are generated using the test that was cleared or approved in conjunction with review of the class II or III ASR. Ordering in-house tests that are developed using analyte specific reagents is limited under section 520(e) of the act to physicians and other persons authorized by applicable State law to order such tests.

III. In vitro Diagnostic Detection Assays

In some embodiments, assays for detecting genetic variation are marketed as in vitro diagnostic tests. The marketing of such kits in the United States requires approval by the Food and Drug Administration (FDA). The FDA classifies in vitro diagnostic kits as medical devices. As such, the pre-market applications for most in vitro diagnostics are submitted to the FDA under the 510(k) regulations and are referred to as 510(k) applications. The 510(k) regulations specify categories for which information should be included.

Each person who wants to market Class I, II and some III devices intended for human use in the U.S. must submit a 510(k) to FDA at least 90 days before marketing unless the device is exempt from 510(k) requirements. Classification of devices are determined by finding the regulation number that is the classification regulation for each device. This can be accomplished searching the classification database for a part of the device name, or, if the device panel (medical specialty) to which the device belongs is known, going directly to the listing for that panel and identify the device and the corresponding regulation. Links to both database can be found on the web page of the FDA.

A 510(k) is a premarketing submission made to FDA to demonstrate that the device to be marketed is as safe and effective, that is, substantially equivalent (SE), to a legally marketed device that is not subject to premarket approval (PMA). Applicants must compare their 510(k) device to one or more similar devices currently on the U.S. market and make and support their substantial equivalency claims. A legally marketed device is a device that was legally marketed prior to May 28, 1976 (preamendments device), or a device which has been reclassified from Class III to Class II or I, a device which has been found to be substantially equivalent to such a device through the 510(k) process, or one established through Evaluation of Automatic Class III Definition. The legally marketed device(s) to which equivalence is drawn is known as the “predicate” device(s).

Applicants must submit descriptive data and, when necessary, performance data to establish that their device is SE to a predicate device. The data in a 510(k) is to show comparability, that is, substantial equivalency (SE) of a new device to a predicate device. A claim of substantial equivalence does not mean the new and predicate devices must be identical. Substantial equivalence is established with respect to intended use, design, energy used or delivered, materials, performance, safety, effectiveness, labeling, biocompatibility, standards, and other applicable characteristics.

Once the device is determined to be SE, it can then be marketed in the U.S. If the FDA determines that a device is not SE, the applicant may resubmit another 510(k) with new data, file a reclassification petition, or submit a premarket approval application (PMA). The SE determination is usually made within 90 days and is made based on the information submitted by the applicant.

A 510(k) is required when introducing a device into commercial distribution (marketing) for the first time, when proposing a different intended use for a device which is already in commercial distribution, and when there is a change or modification of a device already marketed that could significantly affect its safety or effectiveness.

Information required in an application under 510(k) includes:
1) The in vitro diagnostic product name, including the trade or proprietary name, the common or usual name, and the classification name of the device.
2) The intended use of the product.
3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified.
4) Proposed labels, labeling and advertisements sufficient to describe the in vitro diagnostic product, its intended use, and directions for use. Where applicable, photographs or engineering drawings should be supplied.
5) A statement indicating that the device is similar to and/or different from other in vitro diagnostic products of comparable type in commercial distribution in the U.S., accompanied by data to support the statement.
6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request.
7) A statement that the submitter believes, to the best of their knowledge, that all data and information submitted in the premarket notification are truthful and accurate and that no material fact has been omitted.
8) Any additional information regarding the in vitro diagnostic product requested that is necessary for the FDA to make a substantial equivalency determination. A request for additional information will advise the 510(k) submitter that there is insufficient information contained in the original 510(k) submission for a substantial equivalent determination to be made. In this situation the 510(k) submitter may: (a) submit the requested data or a new 510(k) containing the requested information, or (b) submit a PMA application in accordance with section 515 of the FD&C Act. If the additional information is not submitted within 30 days following the date of the request, the FDA may consider the 510(k) to be withdrawn.

Factors used by FDA reviewers in determining substantial equivalency include:
1) Does the in vitro diagnostic device have the same intended use as a currently marketed device (sometimes referred to as a “predicate device”), e.g., nucleic acid diagnostic assay?
2) Does the in vitro diagnostic device have the same technological characteristics, e.g., nucleic acid probes?
3) If new technological features are present, e.g., DNA probe, monoclonal antibody, do they raise new questions regarding safety and effectiveness?

Additionally, the following questions will be used by FDA reviewers to assess whether an in vitro diagnostic device that includes technological changes is substantially equivalent to a predicate device.
1) Does the in vitro diagnostic device pose the same type of questions about safety and effectiveness as the predicate device?
2) Are there accepted scientific methods for assessing the impact of technological changes on safety and effectiveness, e.g., accuracy, specificity, sensitivity, precision?

Data generated using the system and methods of the present invention provides sufficient information to obtain approval on the detection assays. Prior to the present invention, only a small number of in vitro diagnostic detection assays have been approved. The present invention provides system and methods for producing approved detection assays for the hundreds of most medically relevant markers. As such, the present invention provides the predicate devices for many markers by which future detection assays will be compared. In some embodiments, the present invention provides methods for obtaining regulatory approval of new detection assays by comparing data obtained with the new detection assay (e.g., data obtained using the systems and methods of the present invention) to a predicate device obtained by using the systems and methods of the present invention.

IV. Product Development

The present invention provides systems, computer programs, graphical user interfaces, and methods for ordering, manufacturing, and delivering detection assays. In some preferred embodiments, an electronic detection assay ordering system is provided to facilitate the utilization of systems and methods for acquiring and analyzing biological information (e.g., systems and methods for developing detection assays and for use of detection assays in basic research discovery to facilitate selection and development of clinical detection assays).

The discovery of a new gene sequence suspected of correlating to a disease condition offers a starting point for understanding the correlation and hopefully, of leading to a treatment for the condition. This data is input into the one or more components of the system of the present invention. However, extensive amounts of work need to be conducted before a useful and safe treatment can be obtained. The systems and methods of the present invention provide an efficient and thorough means to accelerate the time between initial discovery and useful treatment, and provide the tools for diagnosis and development of therapies using components of a production facility that provides for the efficient ordering, production, and shipment of detection assays. Prior to the invention there was no way for a researcher or other user to determine if a detection assay was commercially available for a SNP of interest so that research could be conducted. For example, where a mutation (e.g., a single nucleotide polymorphism; “SNP”) is suggested to correlate with a disease, the present invention provides systems for identifying an optimal target sequence from which an assay is developed to detect the presence of the mutation in a sample. The present invention also provides systems and methods for designing and producing a highly accurate detection assay or other detection assays directed to the optimized target sequence. The assay may then be used to detect the mutation in a large number of samples to determine the accuracy of the original proposed correlation and to determine additional information about the mutation (e.g., the allele frequency of the mutation in any desired population, data necessary for obtaining approval for clinical products from regulatory agencies, etc.). Data collected from these experiments is then analyzed and processed by systems and methods of the present invention to facilitate improved target selection, the identification of additional mutations, the identification of additional correlations, and the design of clinical assays for diagnosing the presence of the mutations in subjects (e.g., to identify subjects that are appropriate candidates for a particular type of therapy). All of this data is fed to various components of the invention.

In some embodiments of the present invention, efficient, sensitive detection assays are provided. The assays are used by users (e.g. researchers) to collect test result data from a plurality of samples. Data obtained from the samples is used, among other purposes, to validate the detection assay (e.g. data is returned to the databases of the data management systems of the present invention). Validated data is then fed to the various components of the invention. For example, collected test result data is used to provide evidence necessary to support approval (e.g., FDA approval) of clinical products corresponding to the detection assay, and can be fed to and stored on a database which is a part of one or more components of the invention. In some embodiments, a plurality of detection assays are combined into a panel and the panels are used to simultaneously collect data for multiple genetic markers. The collected data is used to provide evidence necessary to support approval of clinical products corresponding to one or more of the detection assays on the panel, and can be sent from a remote site or sites to any of the components of the present invention for optimization of a detection assay or production thereof. In some embodiments, a party provides detection assays at a reduced cost, at a subsidized cost, or at no cost to users (e.g. researchers), and data collected by the users is used to support development and/or approval of clinical detection assay products by the providing party and is fed to a database that is linked to one of the components of the present invention. In other words, detection assays are produced (e.g. by the methods described above), and shipped to a user a reduced charge in exchange for detection assay result data (e.g. returned to one or more databases of the data management systems of the present invention via the internet). The result data is then used to forecast demand for a certain assay, reagent production need. In yet another variant, the data is fed to the inventory component so that inventory of a particular assay or panel can be regulated, (e.g. increased or decreased accordingly).

In some embodiments, the present invention provides systems, routines and methods for the development of research and clinical diagnostic products using a multi-step process (i.e. product development funnel) and data related thereto. A schematic summary of such a process is shown in FIG. 88. This figure shows four stages of detection assay development from discovery-based detection assays (e.g., identification and characterization of sequences and mutations), to medically associated marker detection assays (e.g., detection assays directed to markers associated directly or indirectly with one or more medically important conditions), to analyte-specific reagent assays, to clinical diagnostic detection assays (e.g., in vitro detection of established clinical markers). The funnel shown in FIG. 88 represents the fact that a large number of markers may be examined in the discovery phase, leading to a sub-set that are appropriate for each of the subsequent phases It is appreciated that detection assay development utilizes databases that form a part of one or more components hereof. A discovery-based detection assay data or designation is correlated to a first group of detection assays and stored on a database, and utilized with routines of various components of the invention. Medically associated marker data or designations for another group of detection assays are stored and utilized in routines associated with components of the invention. The same holds true for analyte-specific reagent data or designations for detection assays and clinical diagnostic data or designations for various detection assays. This data is used in the manufacturing, pricing and inventory processes and routines described herein.

The following section describes how DNA analysis products directed to SNP detection are moved through the funnel. The focus on DNA products and SNP detection is for clarity only. RNA analysis products and other analysis products also find use in the present invention (e.g., for detecting and quantitating gene expression and other RNA levels using the same product strategy, including detection of splice variants and polymorphism variants). FIG. 89 shows a schematic summary of the discovery phase. In this phase, detection assays or one or more variety are directed to the thousands to hundreds of thousands of markers are generated. This data is stored on databases of various components thereof for use in the production processes and web order entry routines and processes described herein. While the association of certain SNPs to particular medical conditions has been determined, association has not been established for the majority of SNPs. The present invention provides a broad menu of assays and assay data that is presented to a prospective customer for purchase. For example, more than 80,000 unique assays applying the INVADER assay technology (Third Wave Technologies, Madison, Wis.) have been developed, manufactured and shipped for genotyping research to associate specific SNPs with predisposition to disease. Many of the assays have been sent to collaborative customers at low cost in exchange for access to collected data and rights to commercialize discoveries made with these collaborators.

FIG. 90 shows a schematic summary of the “Medically Associated” phase. Detection assay data is correlated to medically associated data and stored on storage device communicatively linked to one or more components of the invention. As use of detection assays reveals the potential association of a SNP with a medical condition, it is designated a potential clinical marker and earmarked for inclusion on one or more Medically Associated Panels (e.g., panels comprising a plurality of detection assays directed at two or more distinct markers). This data is used in one or more components of the invention for production or pricing. Using this approach, the association of certain SNPs has been established and panels have been prepared. Detection assays are added for new makers to panels as those markers are associated and moved down the funnel. FIG. 90 shows two types of panels created using the systems and methods described herein, those containing markers specific to certain disease types or fields (e.g., cardiovascular disease, oncology, immunology, metabolic disorders, neurological disorders, musculoskeletal disorders, endocrinology, and other genetic diseases) and large panels (e.g., containing 10 thousand or more markers) directed to all known medically relevant diseases. It is appreciated that data of detection assays for these various disease types are correlated, stored on databases, and used in the production processes and web user interface described herein.

In one variant, researchers using the panels validate the associations of particular genetic markers to specific medical conditions Analyte-Specific Reagents (ASRs) phase). Once an association is valid, the assay is moved one step further down the funnel and, more importantly, into the clinical market. At this point a price point may change for the assay, and appropriate price data points are correlated to other detection assay data. The ASR format permits the use of the assay in clinical settings without full FDA approval as the user, a certified clinical laboratory, validates the assay for the particular use. The format also allows for the generation of demand and the monitoring of demand using routines and data for a clinical marker or set of markers prior to deciding to seek FDA approval to market it as a in-vitro diagnostic tool (See FIG. 91).

In yet another variant, which may include a Diagnostics phase, once sufficient market demand exists for a particular assay, full regulatory approval is sought to market the assay as an in vitro diagnostic (IVD). While IVD products are represented as occupying the smallest part of the funnel, they are the largest potential revenue source, as shown schematically in FIG. 92. At this point new or higher price point data may be correlated to one or more components of the detection assay data. As a detection assay is moved from research to clinical use, the cost to produce it does not increase significantly, while the revenue and profit margin it generates increase exponentially. The assay manufactured and shipped as an IVD is fundamentally the same assay that entered the top of the funnel as a discovery tool (although improvements or changes may be made during the process, as described below).

Examples of products for each of the funnel phases is shown in FIG. 91 for both genotyping and SNP detection of DNA samples (e.g., samples containing genomic DNA) and expression analysis. For the discovery phase, the systems and methods of the present invention have been applied to generate over 80 thousand unique SNP detection assays with the ability to add six to ten thousand, or more, additional unique SNP detection assays per month. In some embodiments, discovery panels are manufactured using the methods and systems herein that are directed to SNP analysis of entire genes or chromosomes. The present invention also provides systems and methods for custom design of detection assays at any phase of the funnel (i.e., custom design of research and clinical detection assays) by an end user or internally at a production facility. For the medically associated phase, specific panels have been developed for DNA analysis and a large number of expression analysis detection assays have been developed or are in development. For custom panels, customers may elect one or more markers of their choosing for use on the panel and input this data from a catalogue or markers presented on the customer order componend. In some embodiments, customers enter their desired panel components into a user interface of a software program and the received data is sent for analysis and production to one or more components of the invention.

In some embodiments, the funnel process is facilitated by a low cost, easy-to-use assay (e.g., the INVADER assay) and a production process that allows substantial numbers of detections assays to be generated using the methods, routines and systems of the present invention. Such assays provide the necessary features (e.g., accuracy, sensitivity, ease-of-use, amenability to high throughput automated analysis, etc.) to allow wide-spread use by researchers, such that sufficient data is collected to process large numbers of detections assays through the funnel process. Widespread data collection results in the assay becoming a standard for use in discovery of the genetic basis of disease and management of personalized medicine strategies. For example, the present invention provides systems and methods to allow regulatory approval of clinical diagnostic products of every suitable marker. Detection assays for which regulatory approval is sought have detection assay data correlated with a regulatory approval designation or data, and may be processed using the systems and methods described herein in a manner that is different from, for example, RUO assays. These assay may undergo more rigorous quality control processes described herein.

In certain embodiments, a disease associated assay for a particular type of condition (e.g. Cardiovascular, DMD, CF, oncology, etc.) is sought to be developed. Disease condition data by be correlated with SNP data or RNA data or detection assay data. This correlated data is then used in one or more components of the present invention. FIG. 93 shows an approach that may be used to develop particular disease associated assays. The approach shown in FIG. 93, or similar approaches, shows how a pool of medically associated SNP assays is first identified (e.g. by the systems of the present invention that allow results of assay use to be collected and analyzed), and then this pool is further processed to develop commercial products. In particular, FIG. 93 shows a Medically Associated Panel (MAP) development track and a Clinical development track, how particular assays move throught the development process, how failed assays are further developed, and how successful assays are marketed (e.g. first as Reasearch Use Only (RUO) assays, and then launed as ASRs and/or in vitro diagnostics (IVD)).

In some embodiments, the present invention provides an ASR fast track development process. One of the barriers to a rapid and facile ASR product development lies in the relatively lengthy time required for some of the candidate ASR's to be researched and developed. The period from identification of an ASR to the time that validation studies can begin has ranged from several months to years. However, the integrated systems and processes of the present invention allow this process to be sped up dramatically.

The rapid identification and evaluation of candidate ASRs may, for example, occur in several stages. Overview of the ASR fast track is presented in FIG. 71. The first step in the process is the identification of “Super SNPs”. Super SNPs are generally those SNPs and/or detection assays that have extraordinary performance characteristics from an aggregate of SNPs or detection assays that have been designed and tested. In preferred embodiments, a screening process like the one shown in FIG. 95 is employed. Preferably, a production databases (including QC performance data) of previously designed and tested SNP assays is employed as the starting point. Using a production database as the starting point has many advantages. For example, the SNPs within the database already are likely to have some importance as they have been chosen by a customer (optionally at the customer order entry component of the invention). Also, employing the QC performance data within the database as an initial screen generally eliminates the need for further development.

Once a Super SNP or set of Super SNPs has been identified, the relevance of the SNP site as an Analyte Specific Reagent (ASR) is then determined. This may be done using databases (e.g. public databases, and those on an internal data management system, see above) and routines to compare the target region of the Super SNPs to these databases. If this database search indicates that this target region has relevance to any number of markets (e.g. clinical ASR and/or reasarch use only ASRs) that SNPs status is changed from Super SNP to ASR/RUO candidate on a database used herein.

Next a market review is performed (see FIG. 94). For example, using market research information, ASR/RUO Candidate products are further evaluated as to which market this candidate is most appropriate. Appropriate designations are made correlating this data to detection assay data. Once an ASR/RUO Candidate has been evaluated as to the proper market area, validation studies are performed.

The present invention further provides production systems for manufacturing, documenting, and labeling detection assay products. In some embodiments, the production systems provide detection assays that meet requirements of federal regulations (e.g., Food and Drug Administration regulations). For example, in some embodiments, production, information tracking and recording, and labeling requirements are configured to meet federal regulations such as 21 CFR 800-1299, including, but not limited to, intended use indicia, proprietary name indicia, established name indicia, quantity indicia, concentration indicia, source indicia, measure of activity indicia, warning indicia, precaution indicia, storage instruction indicia, reconstitution indicia, expiration date indicia, observable indication of alteration indicia, net quantity of contents indicia, number of tests indicia, manufacturer indicia, packer indicia, distributor indicia, lot number indicia, control number indicia, chemical principle indicia, physiological principle indicia, biological principle indicia, mixing instruction indicia, sample preparation indicia (e.g., indication relating to pooled samples), use of instrumentation indicia, calibration indicia, specimen collection indicia, known interfering substances indicia, step by step outline of recommended procedures from reception of specimen to result indicia, indicia indicative for improving performance, indicia indicative for improving accuracy, list of materials indicia, amount indicia, time indicia used to assure accurate results, positive control indicia, negative control indicia, indicia explaining the calculation of an unknown, formula indicia, limitation of procedure indicia, additional testing indicia, pertinent reference indicia, batch indicia, and date of issuance of last revision of label indicia. In some embodiments, the storage instruction indicia comprise temperature indicia and humidity indicia. In some embodiments, the system comprises a device for providing multiple container packaging for the detection assays.

In some embodiments, the quality control component comprises one or more components, including, but not limited to, an electronic document control component, a purchasing control component, a vendor ranking component, a vendor quality ranking component, a database of acceptable supplier, contractors, and consultants, a database comprising electronic purchasing documents, a contamination control component, validated computer software, electronic calibration records for one or more components of the system, a non-conforming detection assay rejection component (e.g., comprising a system for evaluation, segregation and disposition of non-conforming detection assays), a communication component for communication with a production component (e.g., including a non-conformance notifier), and statistical routines to detect a quality problem.

In some embodiments, the system comprises a product identifier component. For example, in some embodiments, the identifier component comprises a system for identifying a detection assay or components thereof through a stage (e.g., receipt stage, production stage, distribution stage, installation stage, etc.). In some embodiments, the identifier component comprises a fail-safe anti-mix up module.

In some embodiments, the system comprises a device master recorder and/or a device history recorder. For example, in some embodiments, the device history recorder comprises data of a detection assay or batch manufacture date, quantity date, quality data, acceptance record data, primary identification label data, and control number data. In some embodiments, the system comprises a quality system recorder, a complaint file recorder, and/or a detection assay tracker.

Exemplary implementation of indicia determination, recording, tracking a labeling are provided below.

In some embodiments, in order to meet product quality and labeling requirements, detection assay components (e.g., oligonucleotides) are tested for purity and/or stability using HPLC or other suitable methods (e.g., mass spectroscopy, capillary electrophoresis). These analytical methods generate a result the correlates to stability (e.g., shelf-life) of the component and allows labeling of products without having to check actual stability of a long time period. Thus, analytical methods are used to provide an immediate indication of product stability.

An exemplary method for quality testing by HPLC is provided below.

HPLC Quality Testing

This protocol is prepared for a high-pressure liquid chromatographic (HPLC) method validation for the analysis of 20-60 base single stranded oligonucleotide samples at PPD Development as defined by the United States Pharmacopoeia (USP) and International Conference of Harmonization (ICH) guidelines.

The oligonucleotide samples can be considered part of a medical test kit that falls under the medical device category of the Code of Federal Regulations (CFR). These samples are a synthetic biological product and specific guidance in this area is not given by the ICH. Consistent with ICH guidance this will be a category IV validation with additional optional demonstration of method capabilities as they relate to release testing requirements for a biotechnology product.

The HPLC analysis of oligonucleotides is not the analytical equivalent of a product assay for a pharmaceutical product. For biotechnology products, the biological assay is the closest established analytical equivalent to the pharmaceutical product assay. Quantification of purity by HPLC is complicated by the fact that biological molecules can contain various substitutions or deletions of bases or amino acids and maintain the same biological activity. It is the established industry standard to treat only molecules that differ in biological activity as impurities. These molecular entities or variants that have properties comparable to the desired product are considered part of the desired product (Q6B, Guidance for the Industry Test Procedures and Acceptance Criteria for Biotechnological and biological Products, August 199 ICH, CDER, CBER, FDA, and USDHSS). Quantification is further complicated by the fact that these large biological molecules differ only slightly in molecular weight and chemical properties..

The HPLC analysis of single stranded oligonucleotides by the method of the present invention provide a retention time identity match with standard material, a chromatographic purity value, and a qualitative chromatographic finger print that reveals failure sequences,, degradation products, and modification of bases. The degradation products and failure sequences are referred to, as a profile because the ICH recognizes that the complex polymeric nature of biological molecules does not normally produce a single characteristic degradation but rather a series of degradation products of differing molecular size. The ICH recommends that manufactures demonstrate a stability-indicating profile (Q6B, Guidance for the Industry: Test Procedures and Acceptance Criteria for Biotechnological and biological Products, August 199 ICH, CDER, CBER, FDA, and USDHSS and Q5C, Quality of Biotechnology Products: Stability Testing of Biotechnology and Biological Products, July 1996 ICH Q5C).

The samples and standards are synthetic oligonucleotides in Tris EDTA buffer. The concentration of the standards is determination using the characteristic extinction coefficient of DNA and the UV absorbance of the sample at 260 nm. These concentration values are specific only to DNA. They are synthesized and qualified by mass spec and chromatographic purity-and are defendable as qualitative standards.

Chromoatographic Conditions

Chromatographic system

| | |
|---|---|
| Column: | DionexDNAPac PA-100 ™ |
| | (4 × 250 mm, SN #2843, 3546P) |
| Column T: | 65° C. (controlled by Timberline Column |
| | Oven #TL-105) |
| Detector: | UV 260 nm |
| Mobile Phase: | Line A - 20 mM NaOAc + 20 mM NaClO₄ |
| | Line B - 20 mM NaOAc + 600 mM NaClO₄ |
| Injection Volume: | 250 μL |
| Sample Concentration: | 0.25 μM total oligonucleotide |
| | concentration, b UV @260 nm |

The Dionex PA-100 column is a pellicular anion exchange column that utilizes a large diameter resin bead (0.13 micron diameter, polystyrene-divinylbenzene) with a non-porous substrate coated with 100 nm quanternary ammonium microbeads. This column is designed for single base resolution of single stranded oligonucleotides up to 60 mer. It can be run under denaturing or non-denaturing conditions. The column is stable up to pH 12.4 or up to 90C. Resolution is achieved with a salt gradient that mediates the affinity of the nucleotides for the stationary phase.

Gradient and Flow Rate Table

| Time (min) | Line A | Line B | Flow Rate |
|---|---|---|---|
| 0.0 | 95.0 | 5.0 | 1.0 ml/min |
| 5.0 | 77.0 | 23.0 | |
| 23.0 | 68.0 | 32.0 | |
| 28.0 | 66.0 | 34.0 | |
| 29.0 | 0.0 | 100.0 | |
| 31.0 | 0.0 | 100.0 | |
| 32.0 | 95.0 | 5.0 | |
| 39.0 | 95.0 | 5.0 | |

In some embodiments, analysis is carried out by calculating the percent relative standard deviation (% RSD) of the area response from six replicate injections of the oligonucleotide sample preparation.
Acceptance criteria: % RSD less than or equal to 5.0.
Retention time, relative retention time, and capacity factor are determined. The capacity factor, k′, for each sample peak in the first injection of standard is determined using the following equation:

$${k}^{′} = \frac{t - {t}_{a}}{{t}_{a}}$$

Where
t=the retention time of the Sample peak.
tₐ=the retention time of non-retained component.

A peak retention-time marker solution (SS30) containing equal amounts of 30, 32, 34, and 36 mer synthetic oligonucleotides is analyzed and evaluated to demonstrate the resolution capability of the method and to select and optimize conditions for a particular product run.

The resolution (Rₛ) for each peak is determined using the following equation,

$${R}_{s} = \frac{2 ( {t}_{2} - {t}_{1} )}{( {twb}_{2} + {twb}_{1} )}$$

and by Separation Factor (α)
α=k′₁/k′₂=(t₂−tₐ)/(t₁−tₐ)
Where
t₁=retention time of the first eluting peak
t₂=retention time of the second eluting peak
tₐ=retention time of the void volume =void volume/flow rate
twb₁=extrapolated width, along the baseline of the first eluting peak
twb₂=extrapolated width, along the baseline of the second eluting peak
(twb is used instead of w because the resolution of these biological samples is always hindered by the presents of the n−1 and n+1 oligonucleotides. The SS30 sample is a set of n−2 olionucleotides with a small amount of n−1 present. Since twb excludes tailing and the overlap from the n±1 the resolution value gives a comparative indication for evaluation and tracking of the resolution) The theoretical plates (N) for each peak in the SS30 standard are calculated using the formula:

$$N = 5.54 {( \frac{{t}_{R}}{{W}_{1 / 2}} )}^{2}$$

Where:
t_{R}=the retention time of each peak
W_{1/2}=the width of each peak measured at half the peak height

For a given product development process, the method is optimized for a number of conditions and produced products are documented as being manufactured under these conditions. Conditions include column temperature (e.g., in a range of 63-67° C.) and amount of acetonitirle in the mobile phase (e.g., in a range of 8-12%). Optimization conditions are selected to obtain specificity (the ability to separate the analyte of interest from other components that may be present in the sample), selectivity (the capacity for separating the analyte of interest from all impurities and degradations products), and chromatographic non-interference (lack of interfering peaks in chromatograms). Optimization conditions are also selected to allow proper characterization of a range of test oligonucleotides, including those exposed to acid (e.g., 0.5 N HCl), HClO₄ (20 mM), light (250 W/m2 for 1-3 hours), and heat (80° C. for 1-21 days).

In some embodiments, a single method is employed for measing oligonucleotide stability, where a number of different oligonucleotides are characterized (e.g., oligonucleotides of different length). For example, it was experimentally determined that the following greadient was able to analyze each of the probe, FRET, and INVADER oligonucleotide of an invasive cleavage assay with good performance.

Gradient and Flow Rate Table

| Time (min) | Line A | Line B | Flow Rate |
|---|---|---|---|
| 0.0 | 95.0 | 5.0 | 1.0 ml/min |
| 1.0 | 77.0 | 23.0 | |
| 23.0 | 70.0 | 32.0 | |
| 26.0 | 68.0 | 34.0 | |
| 28.0 | 0.0 | 100.0 | |
| 32.0 | 0.0 | 100.0 | |
| 34.0 | 95.0 | 5.0 | |
| 38.0 | 95.0 | 5.0 | |

The method was also able to function with 58, 62, 64, and 70-mer oligonucleotides with distinguishable resolution of the n−4 oligonucleotides.

Temperature optimization with the invasive cleavage assay components showed that the oligonucleotides had a weaker affinity for the stationary phase and better resolution with lower temperatures.

Gradient optimization was carried out with oligonucleotides of 58, 62, 64, and 70 nucleotides lengths. The early stage of the originally attempted gradient spends several minutes with mobile phase concentrations that are too weak to achieve much resolution. Conversely, the later stages of the analysis are under mobile phase concentrations that were too strong to achieve optimal resolution of the 50-70 mer oligonucleotides. The first step of the gradient optimization was to determine the critical mobile phase concentration that would best resolve the peaks under isocratic conditions. It was found that the pivotal point was somewhere between 82% and 81% mobile phase A. At 82% mobile phase A, the components elute too quickly. At 81% mobile phase A, the peaks elute too slowly with the final two triplets eluting after the system flush begins at 35 minutes. 83% to 79% gradients provide a good profile of failure sequences and degradation products.

In some embodiments, validation of computer software and detection assay product equipment is carried out and reports are generated (e.g., in an automated fashion) to ensure proper function and meet federal tracking and quality requirements. For example, the function and efficiency of combinations of equipment (e.g., dilute and fill systems comprising an oligonucleotide dilute and fill component, wherein the oligonucleotide dilute and fill component comprises an automated liquid processing device operably linked to a spectrophotometer) is monitored and reports are generated.

In some embodiments, the concentration of labeled oligonucleotides (e.g., oligonucleotides attached to a fluorescent dye, E-tag, or other molecule) is determined by multiplying the measured oligonucleotide concentration by a correction factor that accounts for the presence of the label (e.g., to adjust for the error in the concentration measurement caused by the label). Without the correction factor, the concentration of the labeled oligonucleotide would not be accurately reported for many labels.

An exemplary method for generating and using correction factors is as follows. The oligonucleotide concentration is determined using the absorbance value at 260 nm in phosphate buffer (pH 7.2) and a calculated ε_{Mol}²⁶⁰ value. The nearest neighbor calculation, based on the Gray values (Gray et al., Methods in Enzymology, Volume 246, Chapter 3, Table 1, 21, [1995]) may be used in the method. This example provides a method for correction with a complex labeled oligonucleotide having a linker group between the core oligonucleotide and the fluorescent label.

The correction method is illustrated with the following oligonucleotides:
FRET 14 is a conventional oligonucleotide contain containing quench dye Z28 and reporter dye 6-FAM with a TCT spacer. 5′-Y-TCT-X-AGCCGGTTTTCCGGCTGAGACGGCCTCGCGa-3′
FRET 24 is a conventional oligonucleotide contain containing quench dye Z28 and reporter dye Z35 with a TCT spacer. 5′-Z-TCT-X-AGCCGGTTTTCCGGCTGAGACGTCCGTGGCCTa-3′
(a=hexanediol bulk support, X=Z-28, Y=Z-35, Z=6-FAM)

The strong UV band due to the DNA at circa 260 nm is overlapped by the quench and reporter dyes to some extent. There are two issues that need to be resolved namely;.
1. The absorbance ratio of the maximum of the combined dye spectra in the visible region to that at 260 nm.
2. The way in which the spacer, TCT, sequence is to be handled as part of the nearest neighbor calculation.

Issue 1 is complicated by the fact that the dye spectra are influenced by the spacer and each other. To handle this complexity, the spectra of 4 intermediate compounds as well as the base oligonucleotide and FRET are analyzed, as illustrated below.

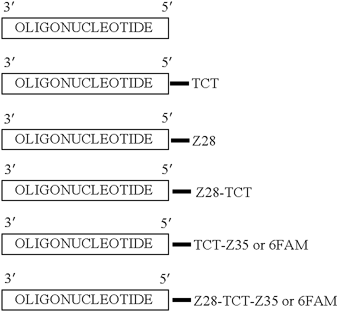

In one embodiment, each of the 6 compounds is made using the normal production and purification processes at the 50 μM scale for both FRETs 14 & 24 and their spectra measured using aqualified diode array spectrophotometer. The software available with the-instrument has sophisticated data manipulation capabilities.

Use of suitable spectral subtraction/normalization and multicomponent analysis techniques allows one to derive a combined dye spectrum from which a factor, F, is calculated. The form of the calculation is:

A correction factor,

$$F = \frac{{A}_{260 n m}^{Dye Pair}}{{A}_{max}^{Dye Pair}} ,$$

is derived from the combined dye spectrum and the corrected absorbance due to the oligonucleotide alone is calculated from

$${A}_{260 nm}^{Corr .} = {A}_{260 nm}^{Meas .} - [ {FA}_{max}^{FRET} ]$$

where
Aₘₐₓ^{FRET} is the absorbance of the FRET at the wavelength on maximum combined dye absorbance and
A_{260 nm}^{Meas.} is the absorbance of the FRET at 260 nm both in pH 7.2 buffer at 25° C.

The correct molar concentration of the FRET, C, is readily calculated from

$$C = \frac{{A}_{260 nm}^{Corr .}}{{ɛ}_{NN} l}$$

ε_{NN} is the molar absorptivity calculated using the nearest neighbor method and using the Gray 1995 values and l is the path length in cm.

All spectra are measured on a qualified Agilent HP8453 diode array spectrophotometer with a Peltier controlled thermostatic cell holder (25±1° C.) in a 10 nm path length quartz flow cell with sipper system using the pH 7.2 buffer solvent as reference. The wavelength range is to be 200 to 700 nm with a spectral bandwidth of 1 nm or better. The spectrophotometer wavelength accuracy is to be confirmed using a holmium perchlorate wavelength standard traceable to NIST SRM 2034. The absorbance accuracy of the instrument is to be confirmed using traceable acidic potassium dichromate standards (Optiglass Ltd., Certificate 12325; NIST SRM 935a) and Burgess Consultancy nicotinic acid standard, EVAL1, at 260 nm (Optiglass 90169).

In some embodiments, detection assays directed to specific subjects (e.g., subject suffering from a particular disease or of a certain identified sub-population) are labeled with warnings about interfering substances that the subject group may be exposed to as a consequence of their medical condition or environment. For example, where a subject is known to or statistically likely to be exposed to certain medications, diets, environmental stresses, etc., appropriate labeling is placed on the detection assays to account for potential interfereing substances.

In some embodiments, documents and reports are managed electronically (e.g., documents and reports corresponding to any of the indicia listed above). For example, all reports required for the detection assays described herein may be generated and managed electronically. As many as thirty separate documents per detection assay may be required to meet regulations. For 1 million distinct assays, this would be 30 million documents. Even if these were single page documents, this would require 60000 reams of paper if the documents were managed in hard copy. In some preferred embodiments, the electronic document management system has a secured access to restrict access to the information to designated personnel. In some preferred embodiments, any modifications of the electronic records are logged and the identify of the modifying party is recorded such that a permanent log is generated (e.g., a log that cannot be deleted).

In some embodiments, each component of a detection assay is tracked such that the supplier or vendor of the component is identified. This is particularly important for panels or arrays, where numerous vendors may have contributed components to a single product. In some embodiments, the quality of the vendor, as well as the identity, is tracked and monitored. For example, quality control data for assays is correlated to particular vendors that supplied a component to the assay, such that a vendor quality rating system is recorded and amended over time consistent with quality control data.

V. Panels, Libraries, Databases

The present invention provides methods and compositions for treating nucleic acid, and in particular, methods and compositions for detection and characterization of nucleic acid sequences and sequence changes. In particular, the present invention provides detection assay panels comprising an array (e.g. microarray) of different detection assays. The arrays are manufactured using the systems and methods described herein. The detection assays include assays for detecting mutations in nucleic acid molecules and for detecting gene expression levels. Assays find use, for example, in the identification of the genetic basis of phenotypes, including medically relevant phenotypes and in the development of diagnostic products, including clinical diagnostic products. The present invention also provides systems and methods for data storage, including data libraries and computer storage media comprising detection assay data.

As discussed above, the present invention is not limited by the nature of the detection assays used in the panels or microarrays of the present invention. A wide variety of available detection technologies find use with the present invention, including those described in detail herein. Purely for illustration purposes, much of the disclosure herein, highlights the use of panels with the INVADER assay detection system (Third Wave Technologies, Madison, Wis.). In particular, the following description provides a detailed analysis of how to apply a detection assay technology (e.g., the INVADER assay) to the systems and methods of the present invention. One skilled in the art will appreciate the applicability of the invention to other detection technologies.

The panels and microarrays of the present invention mark a significant advancement in genetic variation analysis products, allowing researchers to genotype many (e.g., hundreds to thousands) of genetic variations simultaneously in a simple, easy to use, “just add DNA” format. For example, the present invention provides panels comprising a plurality of different INVADER assay detections assays on a single panel. Such panels comprise, for example, the detection assay described in FIG. 96, in U.S. application Ser. No. 10/035,833 filed Dec. 27, 2001 and which is expressly incorporated by reference herein in its entirity, and Tables 10 and 11 which are located on three accompanying CD's, the contents of which are hereby incorporated by reference, both of which include tests for single nucleotide polymorphisms (SNPs) and other mutations that have been associated with diabetes, asthma, deafness, hypertension and other medically relevant conditions.

The panels of the present invention enhance the medical community's ability to detect, catalog and utilize clinically relevant mutations. The availability of disease specific, ready to use panels not only facilitate the additional clinical research needed to extend the initial findings of medical association, but also establish the clinical utility of specific genetic variation analysis products, helping to accelerate their ultimate use and sale as diagnostic tools to the clinical market. Data of which detection assays are part of a respective panel are stored on databases that optionally form part of the components herein and are utilized in the various components of the invention for product presentation, production, inventory control, billing and shipping.

In some embodiments, panels comprise detection assays that allow for simultaneous detection of multiple variations in a sample using identical reaction conditions. For example, the INVADER assay detection panels of the present invention enable scientists to detect multiple genetic variations in one individual using the same array (e.g., microtiter plate) because each well of the plate contains a different SNP or mutation test, all run under identical conditions.

In some preferred embodiments, panels are designed for ease of use. For example, the INVADER assay panels of the present invention are readily produced as products that can be shipped ready to use with stable, dried-down reagents in each reaction site on an array (e.g., each well in a microtiter plate). All the user must do is add genomic or amplified DNA to detect variations in a wide range of genes.

In some preferred embodiments, each detection assay on a panel allows for biplex or multiplex analysis. For example, the INVADER assay may be applied in a biplex format, which enables the simultaneous detection of all variations for each SNP. For example, the presence of the three possible genotypes for an A-C polymorphism—AA, AC or CC—can be determined in a single well. Since each well yields at least one positive signal—A or C or both—the biplex format also provides an internal control.

The panels of the present invention may also be used in conjunction with bioinformatics tools. For example, genetic variation analysis kits comprising the panels of the present invention and software that can be run on virtually all hardware platforms. The bioinformatics software couples the performance and ease of use of the panel product with a data collection and analysis tool. It transforms instrument readings into useful genetic variation data and links it to searchable background information about each detection assay SNP or mutation and additional information available through publicly available databases, including Johns Hopkins' Online Mendelian Inheritance in Man (OMIM) and NCBI's GenBank.

In some embodiments, information pertaining to the panels (e.g., design features, bioinformatics information, test result data, etc.) is collected and stored in one or more databases. Thus, the present invention provide detection assay libraries and searchable databases for use in compiling and analyzing information and for selecting assays for use in future panels and for development of clinical detection assays.

In some embodiments, the panels of the present invention are in microarray format (e.g. oligonucleotdies are Data of which detection assays are part of a respective panel are stored on databases that optionally form part of the components herein and are utilized in the various components of the invention for product presentation, production, inventory control, billing and shipping attached to a solid surface such that a detection assay may be perfored on the solid surface). In other embodiments, the solid support serves as a platform on which microwells are printed/created and the necessary reagents are introduced to these microwells and the subsequent reaction(s) take place entirely in solution. Creation of a microwells on a solid support may be accomplished in a number of ways, including; surface tension, and etching of hydrophilic pockets (e.g. as described in patent publications assigned to Protogene Corp.). For example, the surface of a support may be coated with a hydrophobic layer, and a chemical component, that etches the hydrophobic layer, is then printed on to the support in small volumes. The printing results in an array of hydrophilic microwells. An array of printed hydrophobic towers may be employed to create micorarrays. A surface of of a slide may be coated with a hydrophobic layer, and then a solution is printed on the support that creates a hydrophilic layer on top of the hydrophobic surface. The printing results in an array of hydrophilic towers. Mechanical microwells may be created using physical barriers, ± chemical barriers. For example, microgrids such as gold grids may be immobolized on a support, or microwells may be drilled into the support (e.g. as demonstrated by BML). Also, a microarray may be printed on the support using hydrophilic ink such as TEFLON. Such arrays are commercially available through Precision Lab Products, LLC, Middleton, Wis. In yet another variant, data of customer preferences with respect to the format of the detection assay array are stored on a database used with components of the invention. This information can be used to automatically configure products for a particular customer based upon minimal identification information for a customer, e.g. name, account number or password.

Many types of methods may be used for printing of desired reagents into microwell arrays. In some embodiments, a pin tool is used to load the array mechanically (see, e.g., Shalon, Genome Methods, 6:639 [1996], herein incorporated by reference). In other embodiments, ink jet technology is used to print oligonucleotides onto a solid surface (e.g., O'Donnelly-Maloney et al., Genetic Analysis:Biomolecular Engineering, 13:151 [1996], herein incorporated by reference).

Examples of desired reagents for printing into/onto microwell arrays include, but are not limited to, molecular reagents, such as INVADER reaction reagents, designed to perform a nucleic acid detection assay (e.g., an array of SNP detection assays could be printed in the wells); and target nucleic acid, such as human genomic DNA (hgDNA), resulting in an array of different samples. Also, desired reagents may be simultaneously supplied with the etching/coating reagent or printed into/onto the microwells/towers subsequent to the etching process. For arrays created with mechanical barriers the desired reagents are, for example, printed into the resulting wells. In some embodiments, the desired reagents may need to be printed in a solution that sufficiently coats the microwell and creates a hydrophilic, reaction friendly, environment such as a high protein solution (e.g. BSA, non-fat dry milk). In certain embodiments, the desired reagents may also need to be printed in a solution that creates a “coating” over the reagents that immobilizes the reagents, this could be accomplished with the addition of a high molecular weight carbohydrate such as FICOLL or dextran.

Application of the target solution to the microarray (or reaction reagents if the target has been printed down) may be accomplished in a number of ways. For example, the solid support may be dipped into a solution containing the target or putting the support in a chamber with at least two openings then feeding the target solution into one of the openings and then pulling the solution across the surface with a vacuum or allowing it to flow across the surface via capillary action. Examples of devices useful for performing such methods include, but are not limited to, Tecan—GenePaint system, and AutoGenomics AutoGene System. In yet another embodiment spotters commercially avialable from Virtek Corp. as used to spot various detection assays onto plates, slides and the like.

In some embodiments, solutions (e.g. reaction reagents or target solutions) are dragged, rolled, or squeegeed accross the surface of the support. One type of device useful for this type of application is a framed holder that holds the support. At one end of the holder is a roller/squeegee or something similar that would have a channel for loading of the target solution in front of it. The process of moving the roller/squeegee across the surface applies the target solution to the microwells. At the end opposite end of the holder is a reservoir that would capture the unused target solution (thus allowing for reuse on another array if desired). Behind the roller/squeegee is an evaporation barrier (e.g., mineral oil, optically clear adhesive tape etc.) and it is applied as the roller/squeegee move across the surface.

The application of a target solution to microwell arrays results in the deposition of the solution at each of the microwell locations. The chemical and/or mechanical barriers would maintain the integrity of the array and prevent cross-contamination of reagents from element to element. The reagents printed at each microwell would be rehydrated by the target solution resulting in an ultra-low volume reaction mix. In some embodiments, the microwell-microarray reactions are covered with mineral oil or some other suitable evaporation barrier to allow high temperature incubation. The signal generated may be detected directly through the applied evaporation barrier using a fluorescence microscope, array reader or standard fluorescence plate reader.

Advantages of the use of a microwell-microarray, for running INVADER assays (e.g. dried down INVADER assay components in each well) include, but are not limited to: the ability to use the INVADER Squared (Biplex) format for a DNA detection assay; sufficient sensitivity to detect hgDNA directly, the ability to use “universal” FRET cassettes; no attachment chemistry needed (which means already existing off the shelf reagents could be used to print the microarrays), no need to fractionate hgDNA to account for surface effect on hybridization, low mass of hgDNA needed to make tens of thousands of calls, low volume need (e.g. a 100 μm microwell would have a volume of 0.28 nl, and at 10⁴ microwells per array a volume of 2.8 μl would fill all wells), a solution of 333 ng/μl hgDNA would result in ˜100 copies per microwell (this is 33× more concentrated than the use of 100 ng hgDNA in a 20gl reaction), thus 2.8 μl×333 ng/μl=670 ng hgDNA for 10⁴ calls or 0.07 ng per call. It is appreciated that other detection assays can also be presented in this format.

C. Distribution, Use, and Pricing of Detection Assays

As discussed above, the use of detection assays in the context of research products using the systems and methods of the present invention generates data (which can in one variant be sent automatically over a computer network to one or more components of the present invention) that finds use in obtaining regulatory approval for clinical products and in the generation of databases, which also optionally are used with components of the present invention. In some embodiments of the present invention, a party with interest in selling products (e.g., clinical products) or information stored~in databases provides (e.g., using any delivery systems) detection assays to researchers in order to collect data. In some embodiments, the party provides detection assays to researchers at a reduced cost, at a subsidized cost, or at no cost in order to receive data from said researchers. In yet other embodiments, the party pays a researcher to use the test in order to gain access to data obtained from the test for use in the components hereof. Using the systems and methods of the present invention, the party can compensate for any lost profits or revenues by obtaining and selling clinical products, which are typically high revenue, high margin products.

In one variant of the invention, the system and method of the present invention includes a consumer direct web order entry component (see above). The consumer direct web order entry component provides one or more interactive screens or web pages on a consumer's computer, which is accessible over the Internet or other computer network, from which a consumer can order oligonucleotide detection assay services to be conducted on a genetic sample of the consumer. The consumer can directly order detection assays of the consumer's genetic material or precursor material, e.g. whole blood or other material, through these interactive screens or web pages. In one variant of the invention, the customer can search by allelle frequency. The web pages present the consumer with various assays, panels of detection assays, e.g. a DME panel or screen, or a cardiovascular panel or screen, assays from different manufacturers, and/or combinations thereof. The consumer chooses which detection assay or panel of detection assays the consumer would like to order. The consumer inputs his data on the web page or screen, including but not limited to name, address information, credit card information or other billing or payment information, detection assay, screen or panel selection information from a plurality of different options. This information is then sent to a host computer or server. The host computer or server processes this information and sends the consumer a kit for taking a sample of the consumer's genetic material, e.g. whole blood via a pin prick and collection container, with appropriate identifying markings linking the kit to the consumer and the requisite detection assays or panel(s) requested. The consumer sends back kit with the genetic material or precusor material back to a service provider which then correlates the sample shipment to a predetermined detection assay or panel product, processes the sample, analyzes the sample, and sends the results back to the consumer via the web, e.g. using e-mail, or via a report sent by standard mail. In one alternative of the invention, the consumer logs back on to the web order entry component to access his or her result data by entering a password provided to the consumer upon placement of the intial order or at some latter time.

It is appreciated that this approach provides the consumer with access to personalized medical information, and increases the amount and timeliness of information the consumer is provided with so that informed medical decisions can be made. It is appreciated that the consumer can also have access to an on-line Physician's Desk Reference (“PDR”) (which may be located on the same or different site from that of the consumer direct web order entry component) which has drug information correlated with detection assay information. The Physician's Desk Reference is incorporated herein by reference as if fully set forth. By way of further example, a consumer may be taking a drug which may not be effective to treat the consumer's medical condition. The consumer logs onto the consumer direct web order entry component and enters the name of his drug. He is provided with PDR drug information correlated to detection assay information, e.g. the type of detection assay or panel that should be provided when deciding whether or not to use or prescribe the drug. The consumer then orders the detection assay or detection panel screening service as described above from the service provider, and receives the results of the screen. The results indicate that the consumer has a DME profile such that the drug originally given to the consumer would not be effective or have reduced effectiveness. The consumer is then provided with drug alternatives that are effective for consumer's with this genetic profile. The patient can then approach his physician with this information and seek a prescription for the other drug alternatives and discontinue use of the ineffective drug. It is appreciated that this system and method can also be used proactively prior to the presciption of a drug or drug combination therapy to select the best drug or combination of drugs depending on the consumer's genetic profile. In this variant of the invention, it is appreciated that the PDR is in an electronic format and individual drug entries of information are correlated with data of one or more detection assays or detection assay panel data. In one variant of the invention the PDR forms an integral part of the web order entry component of the invention. In yet another variant, the invention provides a link to the electronic PDR which may be located on another web site.

It is appreciated that the customer order entry component and/or the billing component comprise, in one variant of the invention, a differential pricing component. The differential pricing component is a routine or set of routines that run on one or more computers or other circuitry of the system that provide the ability to price detection assays by the category of detection assay purchased by the consumer or other entity. The billing component may include a secure web based transaction billing routine or software packages, or standard billing routines or software packages commercially available providing billing and tracking functionality. It is also appreciated that the detection assay locator component is periodically update with additional detection assays that are available and are offered for sale.

By way of example, detection assay A or detection assay panel B is either an RUO product,-an ASR product, or an IVD product. It is appreciated that in one version of the invention there is subtantially no difference or no difference between and RUO product, an ASR product, or an IVD product except for price and/or the quality control process the detection assay undergoes, if any. In some embodiments, there is differential pricing for 1) new products (e.g. assays that have not been designed or produced before), 2) low volume products, 3) high volume products, 4) single components of an assay, and 5) an entire kit. In one version of the invention, a customer selects detection assay A or detection assay panel B. The web page then displays a choice between detection assay A-RUO product, detection assay A-ASR product, detection assay B-IVD product. The consumer selects which type of product he desires, e.g. RUO product. The selection is then sent to the remote host computer, and a corresponding RUO product price is presented to the consumer. In another variant, the consumer chooses detection assay A-IVD product. Upon selecting this option the user is display a different price, e.g. an IVD product price. The transaction is then processed. It is also appreciated that that billing component also makes use of this differential pricing feature so that records of the transactions are processed properly. In further embodiments, systems of the present invention also indicate if their is intellectual property (IP) that may cause the prive of the detection assay to increase (e.g. detection assay provided may have paid for a license already, may need to pay a license fee, or may be risking patent litigation through the sale of the assay).

It is also appreciated that the differential pricing routines are capable of pricing the detection assay based upon the platform that the customer selects for the single detection assay or a plurality of detection assays. For example, if a customer selects a 96 well format, price data A are correlated to the detection assay and the transaction is processed. If the customer selects a 384 well format, price data B are correlated to the detection assay and the customer total is appropriately calculated.

D. Medical Records The present invention also relates to medical records (e.g., electronic medical records) comprising genetic information (e.g., patient-specific genetic information) obtained from using one or more of the detection assays produced by the systems and methods described herein. In particular the present invention provides systems and methods for the generation of large amounts of genetic information related to medically relevant conditions and the use of this information in patient health care. For example, the present invention provides systems and methods for generating clinically valid polymorphism data (e.g., SNP data) for any desired subject or population. The data includes information about the presence or absence of the polymorphism in a test subject and a correlation between the presence of a polymorphism or set of polymorphisms and one or more medically relevant conditions. In one variant, this information is generated at a plurality of remote nodes at detection assay user sites and then communicated to one or more central nodes for processing thereof. This information finds use in many aspects of patient health care, including, but not limited to, selection of prescriptions, avoidance of undesired drug reactions or allergic reactions, selection of medical courses of action or therapeutic routes, and the like. Therefore, this information forms a valuable part of the patient's medical records for use in nearly every aspect of patient care. As such, the present invention provides medical records electronically that contain useful genetic information as well as other patient data including, but not limited to prescription data (e.g., data related to one or more drugs or other prescribed medical interventions of the subject, including drug identity, drug reaction data, allergies, risk assessment data, and multi-drug interaction data, billing code levels, order restrictions); information pertaining a physician visit (e.g., date and time of visit, identity of physicians, physician notes, diagnosis information, differential diagnosis information, patient location, patient status, order status, referral information); patient identification information (e.g., patient age, gender, race, insurance carrier, allergies, past medical history, family history, social history, religion, employer, guarantor, address, contact information, patient condition code); and laboratory information (e.g., labs, radiology, and tests).

The genetic information of the present invention may be incorporated into any type of medical record system including electronic medical record systems (e.g., U.S. Pat. Nos. 6,272,468, 6,266,645, 6,263,330, 6,246,975, 6,234,964, 6,206,829, 6,192,112, 6,113,540, 6,088,677, 6,071,236, 6,022,315, 6,006,191, 5,974,398, 5,950,168, 5,924,074, 5,910,107, 5,890,129, 5,867,821, 5,845,255, 5,832,450, 5,823,948, 5,737,539, and PCT Publication Nos. WO 01/54571, WO 00/28460, WO 00/65522, WO 00/29983, WO 00/28459, and WO 99/21114, each of which is herein incorporated by reference in its entirety.

The present invention is not limited by the process of incorporating genetic information into medical records. In some embodiments, genetic information is added to pre-existing medical records, and the data correlated thereto. For example, a subjects electronic medical record is stored on a computer system of a health care professional or an agency that houses data for health care professionals. The genetic information is received by the computer system and stored as part of the medical record. In some embodiments, the genetic information is manually entered into the electronic medical record. In other embodiments, the genetic information is transmitted to the computer system housing the medical record using a communications network (e.g., the Internet). For example, in some embodiments, genetic information (e.g., polymorphism information) is directly transmitted over a communications network from a computer system designed to collect and/or store the genetic information to the computer system housing the medical record. In some embodiments, genetic information is used to create an electronic medical record, wherein additional information pertaining to the subject is added along with, or subsequently, to the medical record.

Genetic information contained in a medical record of the present invention is retrieved and used at any desired time by any desired party. Genetic information, alone, or in combination with other information contained in the medical record, finds use in selecting appropriate health care decisions and courses of action. The health care professional, or other users, evaluate the genetic information, along with other information about the subject in making a informed decision based on all of the circumstances and using the individual's profession judgment. For example, a physician, upon viewing the genetic information and other information contained in the medical record may elect to schedule a medical procedure. Likewise, a pharmacy may elect to prepare a particular type of medication or dose of medication or avoid certain medications based on the information contained in the medical record.

In some embodiments, genetic information is linked to preexisting medical records to enhance the analysis of the genetic information. For example, in some embodiments, a plurality (e.g., thousands) of patient samples are tested to determine one or more genetic characteristics. This genetic information is then compared with the patient's preexisting medical records to determine correlations between the genetic identity and one or more characteristics of the patient contained in the medical record. This allows genetic information (e.g., SNPs) to be correlated to particular medical conditions, drug interactions, gender, race, or other patient characteristics.

In some embodiments of the present information, genetic information contained in a medical record is derived from a biological detection assay, including an indication of the presence or absence of a polymorphism in a subject that is correlated with a medically relevant condition. The present invention is not limited by the identity of the detection assay. For example, in some preferred embodiments, the detection assay is an invasive cleavage assay (e.g., the INVADER assay, Third Wave Technologies, Madison, Wis.) or other detection assay described herein. The present invention provides tens of thousands of designed detection assays (e.g., the INVADER detection assays provided in FIG. 6). The detection assays in FIG. 6 or equivalent assays (e.g., assays targeting similar target sequences, assays using similar probe sequences, non-invasive cleavage assays that use one or more component shown in FIG. 6 or designed based on one or more components shown in FIG. 6, e.g., other hybridization methods using one or more sequences similar to those in FIG. 6) are used to generate genetic information. In other preferred embodiments, other detection assay technologies are used to generate genetic information for use in the medical records of the present invention.

E. Screening Methods for Identifying and Selecting Animal Models

The present invention provides systems and methods for identifying and selecting animal models. In particular, the present invention provides systems and methods for screening animals with a detection assay (e.g. one or more of the detection assays described above) in order to identify animals sharing polymorphisms (e.g. single nucleotide polymorphisms) in the same genes as humans. In this regard, animals that are the most appropriate (e.g. accurate) animal model of a human disease may be employed to screen new or known drug compounds. For example, identifying a species or stain of animal as having a particular polymorphism known to cause drug metabolism problems allows this species or stain to be identified and employed as an animal model to screen candidate drug compounds (e.g. drug compounds that can be metabolized by subjects with a particular polymorphism).

Such animal models sharing a polymorphism with humans allows drugs to proceed through clinical trials in a rapid manner, and allow more effective disease treatment after drug approval, because screening data from these animal models allows human subjects to be either excluded or included in treatment programs. For example, a subject may have a certain polymorphism shared by the animal model indicating that a candidate drug cannot be employed because of efficacy or toxicity concerns. Alternatively, the polymorphism animal model may indicate that treatment is likely to be successful, or even indicate that dosage should be increased or decreased for patients with the particular polymorphisms shared by the animal model. In preferred embodiments, once a species or strain of animal is identified as sharing particular polymorphisms with humans, this animal is used to screen candidate drug compounds by employing individuals with the identified polymorphism, and individuals without the identified polymorphism. In this regard, a comparison may be made between individuals with and without the particular polymorphism.

The present invention also provides methods for screening known animal models (e.g. models for a human disease) in order to identify polymorphisms in these animals. In this regard, the disease the animal is a model for may be correlated with the polymorphisms identified. This also allows polymorphisms in the same or similar genes in humans to be correlated with the actual disease for which the animal is a model. For example, in some embodiments, the methods comprise; a) screening an animal that is a model for a disease in order to identify at least one animal model polymorphism associated with the disease, b) and associating the animal model polymorphism with a human polymorphism in order to identify said human polymorphism as being associated with the same disease, or type of disease, in humans.

In certain embodiments, the present invention provides methods of selecting a non-human animal model for research using human nucleic acid polymorphism detection assays, comprising: using a plurality of genetic detection assays developed for a human to detect nucleic acid genetic variation in an organism other than a human and to obtain organism data; and, comparing the organism data to human nucleic acid polymorphism detection assay data. In some embodiments, the organism data comprises o-polymorphism data, in which the human polymorphism detection assay data comprises h-polymorphism data, and further comprising the step of comparing the h-polymorphism data to the o-polymorphism data. In particular embodiments, the h-polymorphism data comprises data related to a drug metabolizing enzyme gene. In additional embodiments, there is a second organism through an nth organism, where n is an integer greater than or equal to three, and further comprising using a plurality of genetic detection assays developed for the human to determine o-polymorphism data in the second organism through the nth organism; and, comparing the o-polymorphism data for the second organism through the nth organism with the h-polymorphism data.

In some embodiments, the organism data comprises o-SNP data, in which the human genetic detection assay data comprises h-SNP data, and further comprising step of comparing the h-SNP data to the o-SNP data. In additional embodiments, there is a second organism through an nth organism, where n is an integer greater than or equal to three, and further comprising using a plurality of genetic detection assays developed for the human to obtain o-SNP data for the second organism through the nth organism; and, comparing the o-SNP second organism data through the o-SNP nth organism data with the h-SNP data. In certain embodiments, the h-SNP data comprises data related to a drug metabolizing enzyme gene. In additional embodiments, the organism data comprises o-gene expression data, in which the genetic detection assay data comprises h-gene expression data, and further comprising step of comparing the h-gene expression data to the o-gene expression data.

In certain embodiments, there is a second organism through an nth organism, where n is an integer greater than or equal to three, and further comprising using a plurality of genetic detection assays developed for the human to obtain o-gene expression data for the second organism through the nth organism; and, comparing the o-gene expression second organism data through the o-gene expression nth organism data with the h-gene expression data. In some embodiments, the h-gene expression data comprises data related to expression of a drug metabolizing enzyme gene. In further embodiments, the organisms comprise organisms within a single species. In particular embodiments, the method further comprises selecting one of the organisms as the non-human animal model based upon a result of the comparing step. In particular embodiments, the method further comprises executing a routine (e.g. computer software routine) for determining which organisms genetic profile most closely resembles a human genetic profile. In some embodiments, the human genetic profile is selected from a profile for a single gene, a profile for more than one gene, a profile of a metabolic pathway, a profile of sequence homology, a profile of drug metabolizing enzyme genetic sequence homology, and a profile of extent of sequence homology.

In particular embodiments, the methods further comprise developing an organism genetic profile using one or more routines and the organism data. In some embodiments, the organisms comprise organisms within different species. In additional embodiments, the methods further comprise selecting one of the organisms as the non-human animal model based upon a result of the comparing step. In other embodiments, the methods further comprise executing a routine for determining which organisms genetic profile most closely resembles a human genetic profile. In certain embodiments, the human genetic profile is selected from a profile for a single gene, a profile for more than one gene,-a profile of a metabolic pathway, a profile of sequence homology, a profile of drug metabolizing enzyme genetic sequence homology, and a profile of extent of sequence homology. In some embodiments, the organisms comprise organisms within a single species. In further embodiments, the method further comprises selecting one of the organisms as the non-human animal model based upon a result of the comparing step.

In some embodiments, the method further comprises executing a routine for determining which organisms genetic profile most closely resembles a human genetic profile. In particular embodiments, the human genetic profile is selected from a profile for a single gene, a profile for more than one gene, a profile of a metabolic pathway, a profile of sequence homology, a profile of drug metabolizing enzyme genetic sequence homology, a profile of extent of sequence homology. In other embodiments, the methods further comprise selecting one of the organisms as the non-human animal model based upon a result of the comparing step. In additional embodiments, the organisms comprise organism within different species. In particular embodiments, the organisms comprise organism within a single species.

In further embodiments, the methods further comprise selecting one of the organisms as the non-human animal model based upon a result of the comparing step. In some embodiments, the method further comprises executing a routine for determining which organisms genetic profile most closely resembles a human genetic profile. In additional embodiments, the human genetic profile is selected from a profile for a single gene, a profile for more than one gene, a profile of a metabolic pathway, a profile of sequence homology, a profile of drug metabolizing enzyme genetic sequence homology, and a profile of extent of sequence homology.

In some embodiments, the present invention provides methods of selecting a non-human organism model for research using human nucleic acid polymorphism detection assays, comprising: using a plurality of nucleic acid polymorphism detection assays developed for a human to detect nucleic acid variation in an organism other than a human and to obtain organism data; and, using the organism data to develop an organism genetic profile. In certain embodiments, the methods further comprise using the organism genetic profile to select the non-human organism model.

In certain embodiments, the present invention provides methods of research, comprising: selecting an animal model described above; and conducting research related to a drug or drug candidate using the non-human organism model. In further embodiments, the method further comprises administering a drug to the organism, and analyzing a reaction of the organism to the drug.

In some embodiments, the present invention provides methods of conducting an experiment using first organism data, comprising: using a plurality of genetic detection assays developed for a first organism on one of more samples from a second organism, the first organism belonging to a different taxonomic group than the second organism, to obtain second organism data; and, comparing the second organism data with the first organism data. In certain embodiments, the different taxonomic group is selected from a different kingdom, a different phylum, a different class, a different order, a different family, a different genus, a different species, and a different sub-species.

In further embodiments, the first organism is a human and the second organism is a mammal. In particular embodiments, the mammal is a primate. In other embodiments, the mammal is a mouse or rat. In some embodiments, the genetic detection assays are selected from the group consisting of drug metabolizing enzyme genetic detection assays. In certain embodiments, the step of comparing further comprises observing the presence, absence or amount of genetic detection assay signal generated. In certain embodiments, the step of comparing further comprises observing the presence, absence or amount of genetic detection assay signal generated as a percentage of the genetic detection assays used.

In certain embodiments, the present invention provides computer storage media comprising: o-polymorphism data, o-SNP data, and/or o-gene expression data for more than one organism within a single or more than one kingdom, within a single or more than one phylum, within a single or more than class, a single or more than one order, within a single or more than one family, within a single or-more than one genus, or within a single or more than one species. In some embodiments, the present invention provides a computer, computer system or computer network comprising the computer storage medium described above. In particular embodiments, the present invention provides routines for comparing-the data of the computer storage medium described above with second organism data. In further embodiments, the second organism data comprises: o-polymorphism data, o-SNP data, or o-gene expression data for the second organism.

In some embodiments,“the second organism is within the same or different kingdom than the first organism, within the same or different phylum than the second organism, within the same or different class than the second organism, the same or different order than the second organism, within the same or different family than the second organism, within the same or different genus than the second organism, within a same or different species than the second organism, or within the same or different species than the second organism.

In certain embodiments, the detection assay comprises a hybridization assay, a TAQMAN assay, or an invasive cleavage assay. In some embodiments, the detection assay comprises mass spectroscopy, a microarray, a polymerase chain reaction, a rolling circle extension assay, or a sequencing assay. In further embodiments, the detection assay comprises a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In other embodiments, the methods further comprise using the organism data to obtain a drug metabolizing enzyme profile for the organism.

In some embodiments, the present invention provides methods of using a non-human organism for research, comprising: selecting the non-human organism from a group of non-human organisms based upon a predetermined organism genetic profile, the predetermined organism genetic profile determined by using a plurality of human drug metabolizing enzyme genetic detection assays on an organism of the same species as the non-human organism; administering a drug to the non-human organism; and, assaying the non-human organism with a plurality of human drug metabolizing enzyme nucleic acid detection assays after the administration. In additional embodiments, the human drug metabolizing enzyme genetic detection assays (e.g. as described above) are in the form of a kit, the kit comprising kit members capable of detecting one or more drug metabolizing enzyme polymorphisms. In some embodiments, the detection assay comprises an E-Tag from Aclara Corp, or label described in U.S. Pat. No. 6,001,567, herein incorporated by reference (e.g. fluorescent molecule and linker at the 5′ end of an oligonucleotide). In other embodiments, the detection assay comprises a gene expression detection assay.

In certain embodiments, the present invention provides methods of research, comprising: selecting an animal model using the computer, computer system or computer network described above; and, conducting research related to a drug or drug candidate using the animal model. In other embodiments, the method further comprises administering a drug to the organism, and analyzing a reaction of the organism to the drug. In further embodiments, analyzing a reaction of the organism to the drug comprises determining a gene expression level. In additional embodiments, the non-human organism model comprises a non-human animal model. In some embodiments, the analyzing a reaction of the organism to the drug comprises determining an increase or decrease in gene expression.

In particular embodiments, the present invention provides electronic catalogues of animal models, comprising phenotypic data of a plurality of organisms, one or more of the phenotypic data having correlated thereto human nucleic acid polymorphism profile data. In further embodiments, the present invention provides computer systems comprising the electronic catalogues of the present invention. In other embodiments, the computer systems further comprise a publicly accessible wide area network. In some embodiments, the computer systems further comprise order entry routines, order fulfillment routines, or order payment routines. In other embodiments, the computer system further comprises a paper record generator, the paper record generator capable of transferring the electronic catalogue onto a paper record.

In particular embodiments, the present invention provides methods of selecting a non-human organism model for research, comprising: viewing data representative of human nucleic acid polymorphism data correlated to non-human organism data for one or more non-human organism models on display of a computer or workstation, the computer or workstation being communicatively linked to a publicly or privately accessible computer network from which the data is transferred; and, designating one or more of the non-human organism models using routines on the computer or workstation to obtain designated data. In some embodiments, the method further comprises receiving the designated data from the publicly or privately accessible computer network at a receiving computer. In other embodiments, the methods further comprise processing the designated data. In additional embodiments, the processing further comprises invoicing a customer for purchase of one or more of the non-human organism models.

In certain embodiments, the human nucleic acid polymorphism data comprises data obtained for more than 10 drug metabolizing enzyme nucleic acid markers. In some embodiments, the human nucleic acid polymorphism data comprises data obtained for more than 50 drug metabolizing enzyme nucleic acid markers. In other embodiments, the human nucleic acid polymorphism data comprises data obtained for more than 500 drug metabolizing enzyme nucleic acid markers. In additional embodiments, the human nucleic acid polymorphism data comprises data obtained for more than 1000 drug metabolizing enzyme nucleic acid markers. In further embodiments, the human nucleic acid polymorphism data comprises data obtained for more than 4000 drug metabolizing enzyme nucleic acid markers.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A composition comprising a non-amplified oligonucleotide detection assay configured for detecting at least one polymorphism in UGT1A1.

2. The composition of claim 1, wherein said at least one polymorphism in UGT1A1 is selected from UGT1A1 *6, *27, *28, and the polymorphisms shown in FIG. 103.

3. The composition of claim 1, wherein said a non-amplified oligonucleotide detection assay comprises a primary probe, an INVADER oligonucleotide, a structure specific enzyme, and a FRET cassette.

4. The composition of claim 3, wherein said primary probe comprises a 5′ flap.

5. The composition of claim 1, wherein said at least one polymorphism comprises UGT1A1 *6.

6. The composition of claim 5, wherein said a non-amplified oligonucleotide detection assay comprises at least one sequence shown in FIG. 106.

7. The composition of claim 1, wherein said at least one polymorphism comprises UGT1A1 *27.

8. The composition of claim 5, wherein said a non-amplified oligonucleotide detection assay comprises at least one sequence shown in FIG. 120.

9. The composition of claim 1, wherein said at least one polymorphism comprises UGT1A1 *28.

10. The composition of claim 6, wherein said non-amplified oligonucleotide detection assay comprises at least one sequence shown in FIG. 121.

11. A method comprising;
a) providing:
i) a composition comprising a non-amplified oligonucleotide detection assay configured for detecting a UGT1A1 polymorphism, and
ii) a sample from a subject; and
b) testing said sample with said composition in order to determine if said subject has said UGT1A1 polymorphism.

12. The composition of claim 1, wherein said UGT1A1 polymorphism is selected from UGT1A1 *6; *27, *28, and the polymorphisms shown in FIG. 103.

13. The composition of claim 11, wherein said non-amplified oligonucleotide detection assay comprises a primary probe, an INVADER oligonucleotide, a structure specific enzyme, and a FRET cassette.

14. The composition of claim 13, wherein said primary probe comprises a 5′ flap.

15. The composition of claim 11, wherein said UGT1A1 polymorphism is UGT1A1 *6.

16. The composition of claim 15, wherein said non-amplified oligonucleotide detection assay comprises at least one sequence shown in FIG. 106.

17. The composition of claim 11, wherein said UGT1A1 polymorphism is UGT1A1 *27.

18. The composition of claim 17, wherein said non-amplified oligonucleotide detection assay comprises at least one sequence shown in FIG. 120.

19. The composition of claim 11, wherein said UGT1A1 polymorphism is UGT1A1 *28.

20. The composition of claim 19, wherein said non-amplified oligonucleotide detection assay comprises at least one sequence shown in FIG. 121.
